(19) United States
(12) Patent Application Publication
(10) Pub. No.: US 2025/0381289 A1
(43) Pub. Date: Dec. 18, 2025

(54) EGFR AND C-MET BISPECIFIC BINDING AGENTS, CONJUGATES THEREOF AND METHODS OF USING THE SAME

(71) Applicant: GENMAB A/S, Valby (DK)

(72) Inventors: Yang XIAO, Suzhou (CN); Zhu CHEN, Woodinville, WA (US); Baiteng ZHAO, Woodinville, WA (US); Xuan QIU, Suzhou (CN); Jianhe YIN, Suzhou (CN)

(21) Appl. No.: 19/065,063

(22) Filed: Feb. 27, 2025

Related U.S. Application Data
(60) Provisional application No. 63/559,838, filed on Feb. 29, 2024.
(60) Provisional application No. 63/570,775, filed on Mar. 27, 2024.

(51) Int. Cl. A61K 47/68; A61P 35/00; C07K 16/28

(52) U.S. Cl. CPC A61K 47/6849; A61K 47/6803; A61P 35/00; C07K 16/2863; C07K 2317/31; C07K 2317/76; C07K 2317/77; C07K 2317/92; C07K 2317/94

(57) ABSTRACT

The present invention provides EGFR and c-MET bispecific antibodies, antigen binding portions thereof, other binding agents and EGFR and c-MET bispecific conjugates thereof, as well as methods and uses of such antibodies and conjugates for the treatment of cancer and autoimmune disease.

### TECHNICAL FIELD

The present disclosure generally relates to bispecific antibodies and antibody-drug conjugates, as well as methods of using the bispecific antibodies and antibody-drug conjugates, and in particular, to such bispecific antibodies, antibody-drug conjugates, and methods related to diseases and disorders expressing EGFR and/or c-MET.

### BACKGROUND

A great deal of interest has surrounded the use of monoclonal antibodies (mAbs) and bispecific antibodies (BsAbs) for the targeted delivery of cytotoxic agents to cells associated with disease, such as cancer cells and other cells, in the form of antibody drug conjugates (or ADCs). The design of antibody drug conjugates, by attaching a cytotoxic agent, immune modulatory agent or other agent (collectively a “drug”) to an antibody, typically via a linker, involves consideration of a variety of factors. These factors include the identity and location of the chemical group for attachment of the drug, the mechanism of drug release, the structural element(s) (if any) providing release of the drug, and structural modification of the released free drug, if any. If the drug is released in the extracellular environment, the released form of the drug must be able to reach its target. If the drug is to be released after antibody drug conjugate internalization, the structural elements and mechanism of drug release must be consonant with the intracellular trafficking of the conjugate.

Another important factor in the design of antibody drug conjugates is the amount of drug that can be delivered per targeting agent (i.e., the number of drugs attached to each targeting agent (e.g., an antibody), referred to as the drug load or drug loading). Historically, assumptions were that higher drugs loads were superior to lower drug loads (e.g., 8-loads vs 4-loads). The rationale was that higher loaded conjugates would deliver more drug (e.g., cytotoxic agent) to the target cells. This rationale was supported by the observations that conjugates with higher drug loadings were more active against cell lines in vitro. Certain later studies revealed, however, that this assumption was not confirmed in animal models. Conjugates having drug loads of 4 or 8 of certain auristatins were observed to have similar activities in mouse models. See, e.g., Hamblett et al., Clinical Cancer Res. 10:7063-70 (2004). Hamblett et al. further reported that the higher loaded ADCs were cleared more quickly from circulation in animal models. This faster clearance suggested a PK liability for higher loaded species as compared to lower loaded species. See Hamblett et al. In addition, higher loaded conjugates had lower maximum tolerated doses (MTDs) in mice, and as a result had narrower reported therapeutic indices. Id. In contrast, ADCs with a drug loading of 2 at engineered sites in a monoclonal antibody were reported to have the same or better PK and therapeutic indices as compared to certain 4-loaded ADCs. For example, see Junutula et al., Clinical Cancer Res. 16:4769 (2010). Thus, recent trends are to develop ADCs with low drug loadings.

Attractive targets for cancer therapies employing ADCs include EGFR and c-MET. EGFR is a membrane receptor involved in several cell functions such as cell growth and migration, and the overexpression or mutation of EGFR results into tumor formation. c-Met is a membrane receptor which regulates embryonic development and wound healing, and its abnormal activation causes the tumor growth.

EGFR and cMET are frequently co-expressed on tumor cells, often upregulated as the escape mechanism for each other, and both well-validated targets in oncology. Small molecule TKIs and amivantamab have been approved for non-small cell lung cancer with relevant actionable genomic alterations (AGA), and monoclonal antibodies offer treatment options for EGFR-expressing head and neck and colorectal cancers without AGA. Yet high unmet need in these tumors still exists.

There is a need, therefore, for EGFR and c-MET dual-targeting antibodies generally, and for EGFR and cMET dual-targeting ADCs in particular that allow for higher drug loading, but that maintain other characteristics of lower loaded conjugates, such as favorable PK properties. Embodiments of the present invention address these and related needs.

### SUMMARY

Provided herein are EGFR and/or c-MET bispecific binding agents, antibody drug conjugates (ADCs), and methods of using the bispecific binding agents and ADC to treatment diseases such as but not limited to cancers and autoimmune diseases.

In some embodiments, provided is a bispecific binding agent comprising: a first binding domain that binds to EGFR; and a second binding domain that binds to c-MET, wherein the first binding domain comprises a heavy chain and a light chain, the heavy chain comprising a heavy chain variable (VH) region and the light chain comprising a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, wherein the HCDR1 of the first binding domain has an amino acid sequence of SEQ ID NO: 139 or 174, the HCDR2 of the first binding domain has an amino acid sequence of SEQ ID NO: 140 or 175, the HCDR3 of the first binding domain has an amino acid sequence of SEQ ID NO: 141 or 176, the LCDR1 of the first binding domain has an amino acid sequence of SEQ ID NO: 142 or 177, the LCDR2 of the first binding domain has an amino acid sequence of DAS or KVS, and the LCDR3 of the first binding domain has an amino acid sequence of SEQ ID NO: 143 or 178.

In some embodiments, provided herein is a pharmaceutical composition comprising the bispecific binding agent of the present disclosure and a pharmaceutically acceptable carrier.

In some embodiments, provided herein is a nucleic acid encoding the bispecific binding agent of the present disclosure.

In some embodiments, provided herein is a vector comprising the nucleic acid of the present disclosure.

In some embodiments, provided herein is a cell line comprising the bispecific binding agent, the vector, or the nucleic acid of the present disclosure.

In some embodiments, provided herein is a conjugate that comprises the bispecific binding agent, at least one linker attached to the bispecific binding agent; at least one drug unit, wherein each drug unit is attached to a linker, wherein the linker optionally comprises at least one polar group.

In some embodiments, for the conjugate of the present disclosure, the linker is derived from a linker compound, or a stereoisomer or salt thereof. The linker compound comprises the linker unit; a stretcher group connected to the linker unit, an optional amino acid unit; and the at least one polar group. The stretcher group has an attachment site to the bispecific binding agent and an attachment site to the amino acid unit (when present) or the linker unit; the amino acid unit (when present) has an attachment site to the stretcher group and an attachment site to the linker unit; the linker unit has an attachment site to the amino acid unit (when present) or to the stretcher group and an attachment site to the at least one drug unit; and the at least one polar group is attached to at least one of the linker unit, the amino acid unit, or the stretcher group.

In some embodiments, provided herein is a pharmaceutical composition comprising the conjugate of the present disclosure and a pharmaceutically acceptable carrier.

In some embodiments, provided herein is a method of treating an EGFR+ and/or c-MET+ cancer, comprising administering to a subject in need thereof a therapeutically effective amount of the bispecific binding agent, the conjugate, or the pharmaceutical composition of the present disclosure.

In some embodiments, provided herein is a use of the conjugate or the pharmaceutical composition of the present disclosure for the treatment of EGFR+ and/or c-MET+ cancer in a subject.

In some embodiments, provided herein is a conjugate that comprises the binding agent, at least one linker attached to the binding agent; at least one drug unit, wherein each drug unit is attached to a linker, wherein the linker optionally comprises at least one polar group.

In some embodiments, for the conjugate of the present disclosure, the linker is derived from a linker compound, or a stereoisomer or salt thereof, and the linker compound comprises: a linker unit; a stretcher group connected to the linker unit; an optional amino acid unit; and the at least one polar group; wherein: the stretcher group has an attachment site to the binding agent and an attachment site to the amino acid unit (when present) or the linker subunit; the amino acid unit (when present) has an attachment site to the stretcher group and an attachment site to the linker unit; and the linker unit has an attachment site to the amino acid unit (when present) or to the stretcher group and to the at least one drug unit.

In some embodiments, for the conjugate of the present disclosure, the linker compound comprises:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein the polymer unit comprises the formula:

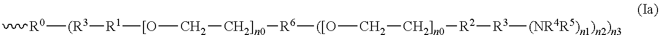

or a stereoisomer or salt thereof, wherein:
R⁰ is a functional group for attachment to a subunit of the amino acid unit;
each R¹ and R² are independently a bond or C₁-C₆ alkylene;
each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, —C₁-C₁₂ alkylene-triazolyl-, —N(polyhydroxyl group)-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R⁶ is selected from:

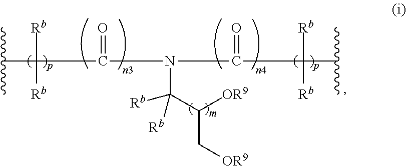

wherein:
   each n³ an n⁴ are independently 0-1,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4,
   each v is independently 1-6, and
n² is 1;

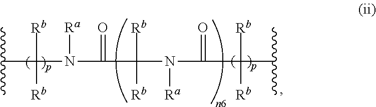

wherein:
   each R^{a} is independently H or C₁₋₆ alkyl,
   each R^{b} is independently H or C₁₋₆ alkyl,
   n⁶ is 1-10,
   each p is independently 0-6, and
n² is 1;

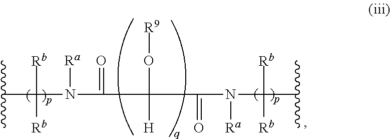

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   q is 1-8,
each v is independently 1-6, and
n² is 1;

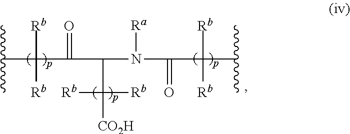

wherein:
   each R^{a} is independently H or C₁₋₆ alkyl,
   each R^{b} is independently H or C₁₋₆ alkyl,
   each p is independently 0-6, and
n² is 1;

—R¹⁰—[O—CH₂—CH₂]₁₋₈—R¹⁰—, wherein: (v)
each R^{b} is independently H or C₁₋₆ alkyl,

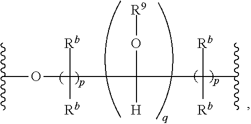

each R¹⁰ is independently H,
each p is independently 1-6,
   each R⁹ is independently H, acetyl, —P(═O)(OH)₂, or —(CH₂)ᵥ—O—S(═O)₂(OH), and
   q is 1-8;
n² is 1; and

—N—(R¹—X—R²—)₂, wherein: (vi)
each X is independently —NR^{a}—C(O)— or —C(O)NR^{a}—, and
n² is 2; and
the wavy line (˜) indicates the attachment site of the amino acid unit to R⁰;
each n⁰ is independently 2-26;
each n¹ is independently 1-6; and
n³ is 1-6.

In some embodiments, for the conjugate of the present disclosure, the linker compound comprises:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises the formula:

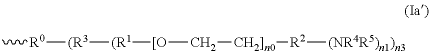

or a stereoisomer or salt thereof, wherein:
R⁰ is a functional group for attachment to a subunit of the amino acid unit;
each R¹ and R² are independently a bond or C₁-C₆ alkylene;
each R³ is independently —N(polyhydroxyl group)-, triazolyl, —C₁-C₁₂ alkylene-triazolyl-,

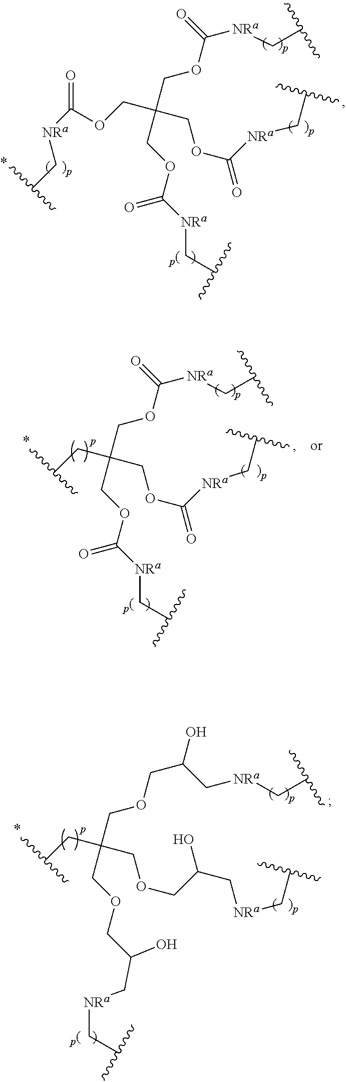

each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;

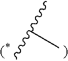

indicates the attachment site of R³ to R⁰
the wavy line

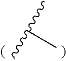

indicates the attachment site of the R³ to R¹;
each p is 1-6;
each n⁰ is independently 2-8;
each n¹ is independently 1-6; and
n³ is 1-6.

In some embodiments, for the conjugate of the present disclosure, the linker compound comprises:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises the formula:

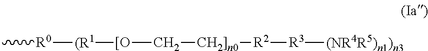

or a stereoisomer or salt thereof, wherein:
(i) R⁰ is a functional group for attachment to a subunit of the amino acid unit;
each R¹ and R² are independently a bond or C₁-C₆ alkylene;
R³ is —C(O)—;
R⁴ is H;
R⁵ is independently a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate; the wavy line (˜) indicates the attachment site of the amino acid unit to R⁰;
n⁰ is independently 2-26;
n¹ is 1-6; and
n³ is 1-6;
(ii) R⁰ is —C(O)—;
R¹, R², and R³ are each a bond;
R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the amino acid unit to R⁰;
n⁰ is 6;
n¹ is 1-6; and
n³ is 1;
(iii) R⁰ is a functional group for attachment to a subunit of the amino acid unit;
R¹ and R² are each, independently, a bond or C₁-C₆ alkylene;
R³ is —NRₐ—C(O)—C₁-C₁₂ alkylene-C(O)—, wherein the alkylene is substituted with —SO₃H;
R^{a} is H or C₁₋₆ alkyl;
R⁴ and R⁵ are each independently H, a carboxyl-containing moiety, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the amino acid unit to R⁰;
each n⁰ is independently 1-26;
n¹ is 1-6; and
n³ is 1-6; or
(iv) R⁰ is

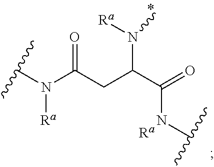

each R¹ is independently a bond or C₁-C₆ alkylene;
R² and R³ are each a bond;
R⁴ and R⁵ are each independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;
the wavy line

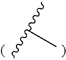

indicates the attachment site of R⁰ to the remainder of the polymer unit; the wavy line (˜*) indicates the attachment site of the amino acid unit to R⁰;
n⁰ is 1-8;
n¹ is 1-6; and
n³ is 2.

In some embodiments, for the conjugate of the present disclosure, the linker compound comprises:
(a) a linker unit having from 1 to 4 attachment sites for a drug unit, said linker unit comprising a moiety of formula:

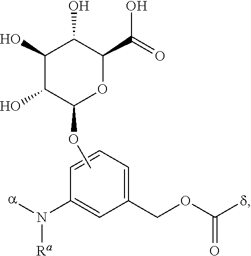

or a stereoisomer or salt thereof, wherein:
α—represents a direct or indirect attachment site to an amino acid unit;
δ—represents an attachment site to at least one of the drug units or for a linking group attached to the at least one of the drug units; and
R^{a} is H or C₁₋₆ alkyl;
(b) the amino acid unit having from 1 to 12 amino acid subunits; and
(c) at least one polar group attached to the amino acid unit, wherein the polar group comprises a polymer unit, optionally a sugar unit, and optionally a carboxyl unit.

In some embodiments, for the conjugate of the present disclosure, the linker compound comprises:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises:
(i) an optionally substituted polyamide comprising the formula

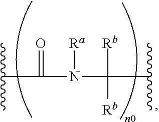

or a stereoisomer thereof, wherein each R^{a} is independently H or C₁₋₆ alkyl and each R^{b} is independently H or C₁₋₆ alkyl, and n⁰ is independently 2-26;
(ii) a substituted polyether comprising the formula

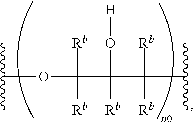

or a stereoisomer thereof, wherein each R^{b} is independently H or C₁₋₆ alkyl, and n⁰ is independently 2-26; or
(iii) combinations thereof.

In some embodiments, for the conjugate of the present disclosure, the linker compound comprises:
(a) the linker unit, which has from 1 to 4 attachment sites for the drug units and having one of the following structures (i) or (ii):

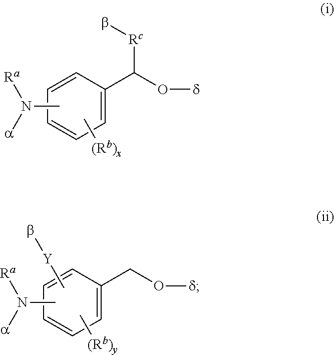

(b) the at least one polar group, each comprises a polymer unit, and
(c) the stretcher group, which has an attachment site for the binding agent;
wherein:
α—is an attachment site to an enzyme-cleavable group;
β—is an attachment site to the at least one polar group;
δ—is H, an attachment site to at least one of the drug units, or an attachment site to a linking group attached to the at least one of the drug units;
the polymer unit comprises a polyamide, a polyether, or a combination thereof, wherein the polyether comprises a hydroxyl group, a polyhydroxyl group, a sugar group, a carboxyl group, or combinations thereof;
each R^{a} independently is H or C₁-C₆ alkyl;
each R^{b} independently is halo, C₁₋₆ alkyl, an attachment site to at least one of the drug units, or
an attachment site to at least one of the polar groups;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl or combinations thereof.

In some embodiments, provided here are conjugates that comprise a drug-linker compound that has one of the structure as described in US Pro. App. No. 63/559,838, which is incorporated by reference in its entirety, or a stereoisomer thereof.

In certain embodiments, the average drug loading (p^{load}) of the conjugate is from about 1 to about 8, about 2, about 4, about 6, about 8, about 10, about 12, about 14, about 16, about 3 to about 5, about 6 to about 8, or about 8 to about 16. In certain embodiments, for the conjugate of the present disclosure, the average p^{load} of the conjugate is about 8. In certain embodiments, for the conjugate of the present disclosure, the average p^{load} of the conjugate is about 5.

In some embodiments, provided here are conjugates as described in US Pro. App. No. 63/559,838, which is incorporated by reference in its entirety.

In some embodiments, provided here are conjugates as follows:

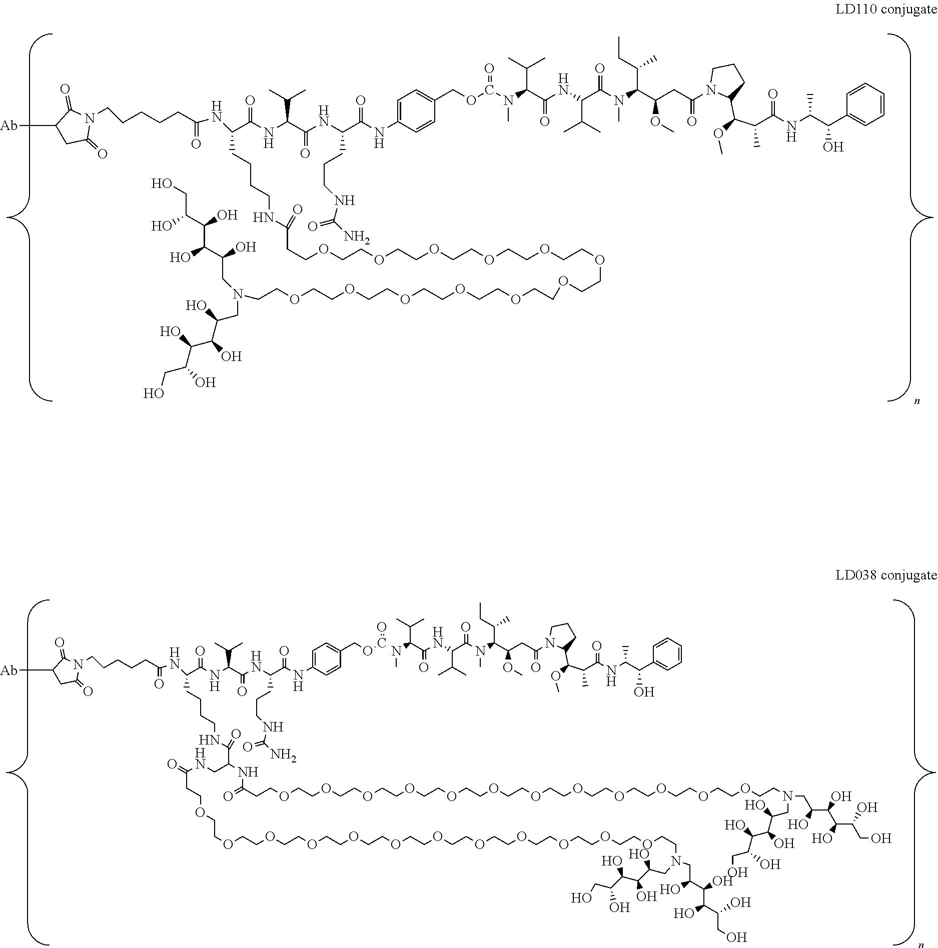

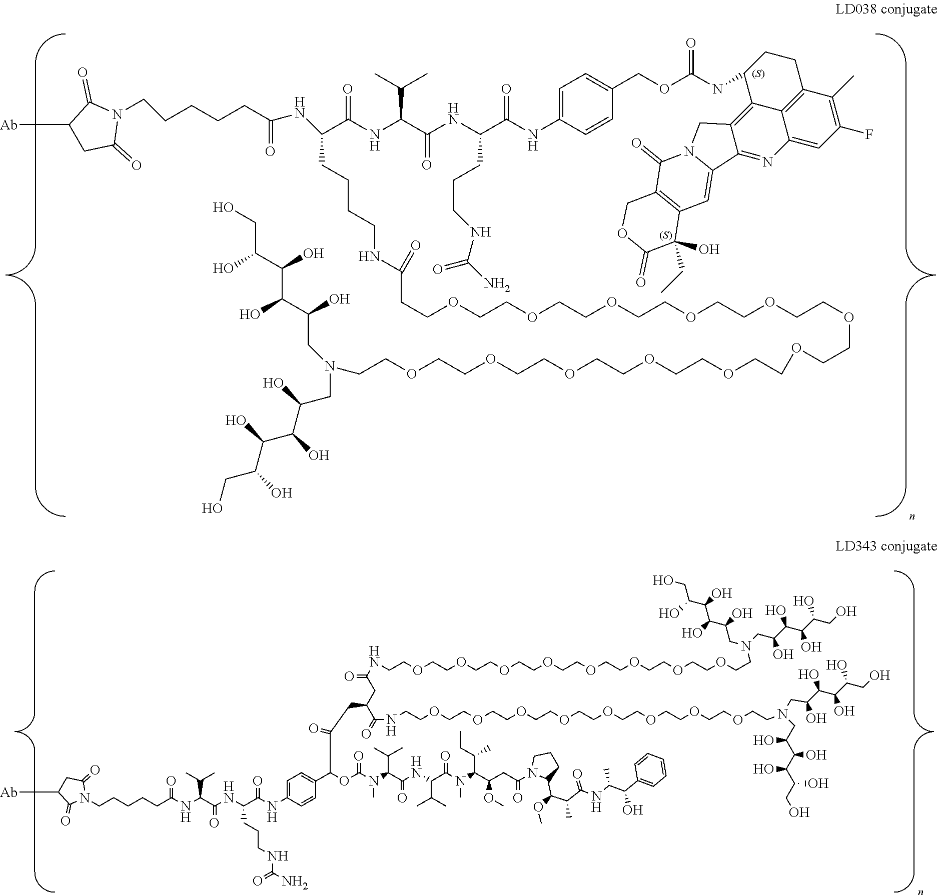

wherein Ab is a binding agent of the present disclosure or included in US Pro. App. No. 63/559,838, which is incorporated by reference in its entirety, or a stereoisomer thereof. In some embodiments, provided herein is a pharmaceutical composition comprising the conjugate of the present disclosure and a pharmaceutically acceptable carrier.

In some embodiments, provided herein is a method of treating an EGFR+ and/or c-MET+ cancer, comprising administering to a subject in need thereof a therapeutically effective amount of the bispecific binding agent, the conjugate, or the pharmaceutical composition of the present disclosure.

In some embodiments, provided herein is a use of the conjugate or the pharmaceutical composition of the present disclosure for the treatment of EGFR+ and/or c-MET+ cancer in a subject.

In some embodiments, provided herein is a method of treating an autoimmune disease, comprising administering to a subject in need thereof a therapeutically effective amount of the conjugate or the pharmaceutical composition of the present disclosure.

In some embodiments, provided herein is a use of the conjugate or the pharmaceutical composition of the present disclosure for the treatment of an autoimmune disease in a subject.

In some embodiments, provided herein is a method of treating an autoimmune disease, comprising administering to a subject in need thereof a therapeutically effective amount of the conjugate or the pharmaceutical composition of the present disclosure.

In some embodiments, provided herein is a use of the conjugate or the pharmaceutical composition of the present disclosure for the treatment of an autoimmune disease in a subject.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1A is a graph illustrating binding activity of parent mAbs on A431 cells;

FIG. 1B is a graph illustrating binding activity of parent mAbs on A549 cells;

FIG. 1C is a graph illustrating binding activity of parent mAbs on MDA-MB-468 cells;

FIG. 1D is a graph illustrating binding activity of parent mAbs on MKN-45 cells;

FIG. 1E is a graph illustrating binding activity of parent mAbs on NCI-H441 cells;

FIG. 1F is a graph illustrating binding activity of parent mAbs on NCI-H1975 cells;

FIG. 2A is a graph illustrating in vitro cytotoxicity of conjugates of parent mAbs and LD038 on EBC-1 cells;

FIG. 2B is a graph illustrating in vitro cytotoxicity of conjugates of parent mAbs and LD038 on EBC-1 cells;

FIG. 2C is a graph illustrating in vitro cytotoxicity of conjugates of parent mAbs and LD038 on A431 cells;

FIG. 2D is a graph illustrating in vitro cytotoxicity of conjugates of parent mAbs and LD038 on MKN-45 cells;

FIG. 3 is a graph illustrating concurrent binding of Bsab 67 for both targets, EGFR and cMET;

FIG. 4A is a graph illustrating HGF blocking test results of Bsab 6, Bsab 67, Bsab 68, and Bsab 81, compared to other antibodies;

FIG. 4B is a graph illustrating EGF blocking test results of Bsab 6, Bsab 67, Bsab 68, and Bsab 81, compared to other antibodies;

FIG. 5A is a graph illustrating binding activity of Bsab 6 and Bsab 12 on EBC-1 cells, compared to other bispecific antibodies;

FIG. 5B is a graph illustrating binding activity of Bsab 6 and Bsab 12 on NCI-H1975 cells, compared to other monospecific antibodies;

FIG. 5C is a graph illustrating binding activity of Bsab 6 and Bsab 12 on MKN-45 cells, compared to other monospecific antibodies;

FIG. 5D is a graph illustrating binding activity of Bsab 6 and Bsab 12 on NCI-H1993 cells, compared to other monospecific antibodies;

FIG. 5E is a graph illustrating binding activity of Bsab 6 and Bsab 12 on A549 cells, compared to other monospecific antibodies;

FIG. 6A is a graph illustrating binding activity of Bsab 6, Bsab 67, Bsab 68, and Bsab 81 on EBC-1 cells, compared to Amivantamab;

FIG. 6B is a graph illustrating binding activity of Bsab 6, Bsab 67, Bsab 68, and Bsab 81 on NCI-H1975 cells, compared to Amivantamab;

FIG. 6C is a graph illustrating binding activity of Bsab 6, Bsab 67, Bsab 68, and Bsab 81 on HCC-827 cells, compared to Amivantamab;

FIG. 6D is a graph illustrating binding activity of Bsab 6, Bsab 52, Bsab 67, and Bsab 68 on MKN-45 cells, compared to other antibodies;

FIG. 6E is a graph illustrating binding activity of Bsab 6, Bsab 52, Bsab 67, and Bsab 68 on NCI-N87 cells, compared to other antibodies;

FIG. 7A is a graph illustrating internalization of Bsab 6, Bsab 67, Bsab 68, and Bsab 81 in A431 cells, compared to other antibodies;

FIG. 7B is a graph illustrating internalization of Bsab 6, Bsab 67, Bsab 68, and Bsab 81 in MNK-45 cells, compared to other antibodies;

FIG. 7C is a graph illustrating internalization of Bsab 6, Bsab 67, Bsab 68, and Bsab 81 in NCI-H1975 cells, compared to other antibodies;

FIG. 8A is a graph illustrating in vitro cytotoxicity of conjugates of Bsab 6, Bsab 67, Bsab 68, and Bsab 81 in EBC-1 cells, compared to other conjugates of antibodies;

FIG. 8B is a graph illustrating in vitro cytotoxicity of conjugates of Bsab 6, Bsab 67, Bsab 68, and Bsab 81 in NCI-H1975 cells, compared to other conjugates of antibodies;

FIG. 8C is a graph illustrating in vitro cytotoxicity of conjugates of Bsab 6, Bsab 67, Bsab 68, and Bsab 81 in HCC-827 cells, compared to other conjugates of antibodies;

FIG. 8D is a graph illustrating in vitro cytotoxicity of conjugates of Bsab 6, Bsab 67, Bsab 68, and Bsab 81 in SNU-5 cells, compared to other conjugates of antibodies;

FIG. 8E is a graph illustrating in vitro cytotoxicity of conjugates of Bsab 6, Bsab 67, Bsab 68, and Bsab 81 in A431 cells, compared to other conjugates of antibodies;

FIG. 8F is a graph illustrating in vitro cytotoxicity of conjugates of Bsab 6, Bsab 67, Bsab 68, and Bsab 81 in MKN-45 cells, compared to other conjugates of antibodies;

FIG. 9A is a graph illustrating anti-proliferation effect of Bsab 67 on A431 cells;

FIG. 9B is a graph illustrating anti-proliferation effect of Bsab 67 on SNU-5 cells;

FIG. 10A is a graph illustrating pharmacokinetics (PK) of Bsab 6 and Basb 12 in rat, compared to Amivantamab;

FIG. 10A is a graph illustrating pharmacokinetics of Bsab 6 WT and Basb 12 WT in rat, compared to Amivantamab;

FIG. 10B is a graph illustrating pharmacokinetics of Bsab 6 WT, Basb 67 WT, Basb 68 WT, and Basb 81 WT in rats;

FIG. 10C is a graph illustrating pharmacokinetics of Basb 67, Basb 81 and conjugates of Basb 67 and Basb 81 in rats;

FIG. 11A is a graph illustrating anti-tumor activity of conjugates of Basb 6 and Basb 12 on EBC-1 cells, compared to other antibodies and conjugates of antibodies;

FIG. 11B is a graph illustrating anti-tumor activity of conjugates of Basb 6 and Basb 12 on NCI-H1975 cells, compared to other antibodies and conjugates of antibodies;

FIG. 12A is a graph illustrating anti-tumor activity of conjugates of Basb 6, Basb 52 WT, Basb 67 WT, Basb 68 WT, and Basb 81 WT on EBC-1 cells, compared to Telisotuzumab vedotin;

FIG. 12B is a graph illustrating anti-tumor activity of conjugates of Basb 6, Basb 67 WT, Basb 68 WT, and Basb 81 WT on MKN-45 cells, compared to other conjugates of antibodies;

FIG. 12C is a graph illustrating anti-tumor activity of conjugates of Basb 6, Basb 67 WT, Basb 68 WT, and Basb 81 WT on SNU-5 cells, compared to other conjugates of antibodies;

FIG. 12D is a graph illustrating anti-tumor activity of conjugates of Basb 6, Basb 67 WT, and Basb 81 WT on SNU-5 cells, compared to other conjugates of antibodies;

FIG. 13A is a graph illustrating anti-tumor activity of conjugates of Basb 67, Basb 81, and Basb 67 WT on NCI-N87 cells, compared to Amivantamab;

FIG. 13B is a graph illustrating anti-tumor activity of Basb 67 WT, Basb 67, Basb 81, and conjugates of Basb 67 and Basb 67 WT on MKN-45 cells, compared to Amivantamab;

FIG. 14 is a graph illustrating anti-tumor activity of conjugates of Basb 67 and Basb 81 on TE4 cells, compared to Amivantamab;

FIG. 15A is a graph illustrating heat stability of Basb 67 and the conjugate thereof (Basb 67-LD038 (8));

FIG. 15B is a graph illustrating heat stability of an antibody (AZD9592-Ab) and the conjugate thereof (AZD9592);

FIG. 16 is a graph illustrating hydrophobic interaction chromatography of Basb 67, Basb 67-LD038 (8), AZD9592-Ab, and AZD9592;

FIG. 17A is a graph illustrating binding activity of Basb 67, Basb 67-LD038 (8), AZD9592-Ab, and amivantamab to Human EGFR;

FIG. 17B is a graph illustrating binding activity of Basb 67, Basb 67-LD038 (8), AZD9592-Ab, and amivantamab to Human HER2;

FIG. 17C is a graph illustrating binding activity of Basb 67, Basb 67-LD038 (8), AZD9592-Ab, and amivantamab to Human HER3;

FIG. 17D is a graph illustrating binding activity of Basb 67, Basb 67-LD038 (8), AZD9592-Ab, and amivantamab to Human HER4;

FIG. 17E is a graph illustrating binding activity of Basb 67, Basb 67-LD038 (8), AZD9592-Ab, and amivantamab to Human cMET;

FIG. 17F is a graph illustrating binding activity of Basb 67, Basb 67-LD038 (8), AZD9592-Ab, and amivantamab to Human Sema3A;

FIG. 18A is a graph illustrating binding activity of Basb 67, Basb 67-LD038 (8), AZD9592-Ab, and amivantamab on A431 cells;

FIG. 18B is a graph illustrating binding activity of Basb 67, Basb 67-LD038 (8), AZD9592-Ab, and amivantamab on SW620 cells;

FIG. 18C is a graph illustrating binding activity of Basb 67, Basb 67-LD038 (8), AZD9592-Ab, AZD9592, and amivantamab on EBC-1 cells;

FIG. 18D is a graph illustrating binding activity of Basb 67, Basb 67-LD038 (8), AZD9592-Ab, AZD9592, and amivantamab on SNU-5 cells;

FIG. 18E is a graph illustrating binding activity of Basb 67 and Basb 67-LD038 (8) on CAL-27 cells;

FIG. 18F is a graph illustrating binding activity of Basb 67 and Basb 67-LD038 (8) on KYSE-30 cells;

FIG. 18G is a graph illustrating binding activity of Basb 67 and Basb 67-LD038 (8) on MKN-45 cells;

FIG. 18H is a graph illustrating binding activity of Basb 67 and Basb 67-LD038 (8) on HT-29 cells;

FIG. 18I is a graph illustrating binding activity of Basb 67 on NCI-H292 cells;

FIG. 18J is a graph illustrating binding activity of Basb 67 on THP-1 cells;

FIG. 19A is a graph illustrating internalization of Bsab 67 in various tumor cell lines;

FIG. 19B is a graph illustrating internalization of Bsab 67-LD038 (8) in various tumor cell lines;

FIG. 20A is a graph illustrating in vitro cytotoxicity of Bsab 67-LD038 (8), AZD9592, Exatecan, and b12-LD038 (8) on NCI-H292 cells;

FIG. 20B is a graph illustrating in vitro cytotoxicity of Bsab 67-LD038 (8), AZD9592, Exatecan, and b12-LD038 (8) on NCI-H1975 cells;

FIG. 20C is a graph illustrating in vitro cytotoxicity of Bsab 67-LD038 (8), AZD9592, Exatecan, and b12-LD038 (8) on EBC-1 cells;

FIG. 20D is a graph illustrating in vitro cytotoxicity of Bsab 67-LD038 (8), AZD9592, Exatecan, and b12-LD038 (8) on CAL-27 cells;

FIG. 20E is a graph illustrating in vitro cytotoxicity of Bsab 67-LD038 (8), AZD9592, Exatecan, and b12-LD038 (8) on KYSE-30 cells;

FIG. 20F is a graph illustrating in vitro cytotoxicity of Bsab 67-LD038 (8), AZD9592, Exatecan, and b12-LD038 (8) on KYSE-150 cells;

FIG. 20G is a graph illustrating in vitro cytotoxicity of Bsab 67-LD038 (8), AZD9592, Exatecan, and b12-LD038 (8) on MKN-45 cells;

FIG. 20H is a graph illustrating in vitro cytotoxicity of Bsab 67-LD038 (8), AZD9592, Exatecan, and b12-LD038 (8) on SNU-5 cells;

FIG. 20I is a graph illustrating in vitro cytotoxicity of Bsab 67-LD038 (8), AZD9592, Exatecan, and b12-LD038 (8) on HT-29 cells;

FIG. 20J is a graph illustrating in vitro cytotoxicity of Bsab 67-LD038 (8), AZD9592, Exatecan, and b12-LD038 (8) on THP-1 cells;

FIG. 21 is a graph illustrating bystander effect of Bsab 67 in target positive cells KYSE-30 and EGFR & cMET negative cells THP-1-Luc;

FIG. 22A is a graph illustrating anti-tumor activity of Bsab 67-LD038 (8), AZD9592, and amivantamab on CAL-27 cells;

FIG. 22B is a graph illustrating anti-tumor activity of Bsab 67-LD038 (8), Bsab 67-LD343 (8), and amivantamab on KYSE-30 cells;

FIG. 22C is a graph illustrating anti-tumor activity of Bsab 67-LD038 (8) on MDA-MB-468 cells;

FIG. 22D is a graph illustrating anti-tumor activity of Bsab 67-LD038 (8) on FADU cells;

FIG. 22E is a graph illustrating anti-tumor activity of Bsab 67-LD038 (8), AZD9592, and amivantamab on MKN-45 cells;

FIG. 22F is a graph illustrating anti-tumor activity of Bsab 67-LD038 (8) on SNU-5 cells;

FIG. 23A is a graph illustrating anti-tumor activity of Bsab 67-LD038 (8), AZD9592, and amivantamab on HT-55 cells;

FIG. 23B is a graph illustrating anti-tumor activity of Bsab 67-LD038 (8) on Detroit 562 cells;

FIG. 23C is a graph illustrating anti-tumor activity of Bsab 67-LD038 (8), AZD9592, and amivantamab on HT-29 cells;

FIG. 23D is a graph illustrating anti-tumor activity of Bsab 67-LD038 (8), Bsab 67-LD343 (8), and amivantamab on KYSE-150 cells;

FIG. 23E is a graph illustrating anti-tumor activity of Bsab 67-LD038 (8) on NCI-N87 cells;

FIG. 23F is a graph illustrating anti-tumor activity of Bsab 67-LD038 (8) on TE-4 cells at various dosages; FIG. 24A is a graph illustrating anti-tumor activity of Bsab 67-LD038 (8) and Bsab 67-LD343 (8) on NCI-H1975 cells at various dosages;

FIG. 24B is a graph illustrating anti-tumor activity of Bsab 67-LD038 (8) and Bsab 67-LD343 (8) on MKN-45 cells at various dosages;

FIG. 24C is a graph illustrating anti-tumor activity of Bsab 67-LD038 (8) and Bsab 67-LD343 (8) on TE4 cells at various dosages;

FIG. 25A is a graph illustrating pharmacokinetics of Bsab 67, Bsab 67-LD038 (8), AZD9592-Ab, and AZD9592 in rats;

FIG. 25B is a graph illustrating pharmacokinetics of Bsab 67-LD038 (8) in NCI-N87 tumor-bearing Balb/c mice;

FIG. 25C is a graph illustrating pharmacokinetics of Bsab 67, Bsab 67-LD038 (8), AZD9592-Ab, and AZD9592 in NOD-SCID mice;

FIG. 25D is a graph illustrating pharmacokinetics of Bsab 67-LD038 (8) in cynomolgus monkeys;

FIG. 26A is a graph illustrating heat stability of Basb 67 and the conjugate thereof (Basb 67-LD038 (5));

FIG. 26B is a graph illustrating heat stability of an antibody (AZD9592-Ab) and the conjugate thereof (AZD9592);

FIG. 27 is a graph illustrating hydrophobic interaction chromatography of Basb 67, Basb 67-LD038 (5), AZD9592-Ab, and AZD9592;

FIG. 28A is a graph illustrating binding activity of Basb 67, Basb 67-LD038 (5), AZD9592-Ab, and amivantamab to Human EGFR;

FIG. 28B is a graph illustrating binding activity of Basb 67, Basb 67-LD038 (5), AZD9592-Ab, and amivantamab to Human HER2;

FIG. 28C is a graph illustrating binding activity of Basb 67, Basb 67-LD038 (5), AZD9592-Ab, and amivantamab to Human HER3;

FIG. 28D is a graph illustrating binding activity of Basb 67, Basb 67-LD038 (5), AZD9592-Ab, and amivantamab to Human HER4;

FIG. 28E is a graph illustrating binding activity of Basb 67, Basb 67-LD038 (5), AZD9592-Ab, and amivantamab to Human cMET;

FIG. 28F is a graph illustrating binding activity of Basb 67 and Basb 67-LD038 (5) to Human Sema3A;

FIG. 29A is a graph illustrating binding activity of Basb 67, Basb 67-LD038 (5), AZD9592-Ab, AZD9592, Amivantamab, Zalutumumab, and Telisotuzumab on EBC-1 cells;

FIG. 29B is a graph illustrating binding activity of Basb 67, Basb 67-LD038 (5), AZD9592-Ab, AZD9592, Amivantamab, Zalutumumab, and Telisotuzumab on NCI-H292 cells;

FIG. 29C is a graph illustrating binding activity of Basb 67, Basb 67-LD038 (5), AZD9592-Ab, AZD9592, Amivantamab, Zalutumumab, and Telisotuzumab on NCI-H1975 cells;

FIG. 29D is a graph illustrating binding activity of Basb 67, Basb 67-LD038 (5), AZD9592-Ab, AZD9592, Amivantamab, Zalutumumab, and Telisotuzumab on CAL-27 cells;

FIG. 29E is a graph illustrating binding activity of Basb 67, Basb 67-LD038 (5), AZD9592-Ab, AZD9592, Amivantamab, Zalutumumab, and Telisotuzumab on KYSE-150 cells;

FIG. 29F is a graph illustrating binding activity of Basb 67, Basb 67-LD038 (5), AZD9592-Ab, AZD9592, Amivantamab, Zalutumumab, and Telisotuzumab on SNU-5 cells;

FIG. 29G is a graph illustrating binding activity of Basb 67, Basb 67-LD038 (5), AZD9592-Ab, AZD9592, Amivantamab, Zalutumumab, and Telisotuzumab on HT-29 cells;

FIG. 29H is a graph illustrating binding activity of Basb 67, Basb 67-LD038 (5), AZD9592-Ab, AZD9592, Amivantamab, Zalutumumab, and Telisotuzumab on ACHN cells;

FIG. 29I is a graph illustrating binding activity of Basb 67, Basb 67-LD038 (5), AZD9592-Ab, AZD9592, Amivantamab, Zalutumumab, and Telisotuzumab on THP-1 cells;

FIG. 30A is a graph illustrating internalization of Bsab 67 in various tumor cell lines;

FIG. 30B is a graph illustrating internalization of Bsab 67-LD038 (5) in various tumor cell lines;

FIG. 31A is a graph illustrating in vitro cytotoxicity of Bsab 67-LD038 (5), AZD9592, Exatecan, and b12-LD038 (8) on NCI-H292 cells;

FIG. 31B is a graph illustrating in vitro cytotoxicity of Bsab 67-LD038 (5), AZD9592, Exatecan, and b12-LD038 (8) on NCI-H1975 cells;

FIG. 31C is a graph illustrating in vitro cytotoxicity of Bsab 67-LD038 (5), AZD9592, Exatecan, and b12-LD038 (8) on EBC-1 cells;

FIG. 31D is a graph illustrating in vitro cytotoxicity of Bsab 67-LD038 (5), AZD9592, Exatecan, and b12-LD038 (8) on CAL-27 cells;

FIG. 31E is a graph illustrating in vitro cytotoxicity of Bsab 67-LD038 (5), AZD9592, Exatecan, and b12-LD038 (8) on KYSE-30 cells;

FIG. 31F is a graph illustrating in vitro cytotoxicity of Bsab 67-LD038 (5), AZD9592, Exatecan, and b12-LD038 (8) on KYSE-150 cells;

FIG. 31G is a graph illustrating in vitro cytotoxicity of Bsab 67-LD038 (5), AZD9592, Exatecan, and b12-LD038 (8) on MKN-45 cells;

FIG. 31H is a graph illustrating in vitro cytotoxicity of Bsab 67-LD038 (5), AZD9592, Exatecan, and b12-LD038 (8) on SNU-5 cells;

FIG. 31I is a graph illustrating in vitro cytotoxicity of Bsab 67-LD038 (5), AZD9592, Exatecan, and b12-LD038 (8) on HT-29 cells;

FIG. 31J is a graph illustrating in vitro cytotoxicity of Bsab 67-LD038 (5), AZD9592, Exatecan, and b12-LD038 (8) on ACHN cells;

FIG. 31K is a graph illustrating in vitro cytotoxicity of Bsab 67-LD038 (5), AZD9592, Exatecan, and b12-LD038 (8) on THP-1 cells;

FIG. 32A is a graph illustrating bystander effect of Bsab 67 in target positive cells EBC-1 and EGFR & cMET negative cells THP-1-Luc;

FIG. 32B is a graph illustrating bystander effect of Bsab 67 in target positive cells KYSE-30 and EGFR & cMET negative cells THP-1-Luc;

FIG. 33A is a graph illustrating anti-tumor activity of Bsab 67-LD038 (5) and AZD9592 on EBC-1 cells at various dosages;

FIG. 33B is a graph illustrating anti-tumor activity of Bsab 67-LD038 (5), AZD9592, and Amivantamab on CAL-27 cells at various dosages;

FIG. 33C is a graph illustrating anti-tumor activity of Bsab 67-LD038 (5), AZD9592, and Amivantamab on NCI-H1975 cells at various dosages;

FIG. 33D is a graph illustrating anti-tumor activity of Bsab 67-LD038 (5), AZD9592, and Bsab 67-LD038 (8) on MKN-45 cells at various dosages;

FIG. 34A is a graph illustrating anti-tumor activity of Bsab 67-LD038 (5), AZD9592, and Amivantamab on NCI-H292 cells at various dosages;

FIG. 34B is a graph illustrating anti-tumor activity of Bsab 67-LD038 (5), AZD9592, and Amivantamab on KYSE-150 cells at various dosages;

FIG. 34C is a graph illustrating anti-tumor activity of Bsab 67-LD038 (5), AZD9592, and Amivantamab on HT-29 cells at various dosages;

FIG. 34D is a graph illustrating anti-tumor activity of Bsab 67-LD038 (5), AZD9592, and Amivantamab on ACHN cells at various dosages;

FIG. 35A is a graph illustrating pharmacokinetics of Bsab 67 and Bsab 67-LD038 (5) in rats;

FIG. 35B is a graph illustrating pharmacokinetics of Bsab 67-LD038 (5) and AZD9592 in CAL-27 tumor-bearing NOD-SCID mice;

FIG. 35C is a graph illustrating pharmacokinetics of Bsab 67-LD038 (5) and AZD9592 in NCI-H1975 tumor-bearing Balb/c mice; and

FIG. 36 is a graph illustrating plasma stability of Bsab 67-LD038 (5) in human, cyno (cynomolgus monkey), mouse, and rat plasma.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” and/or “including” when used in this specification, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawing(s), all of which form a part of this specification. It is to be expressly understood, however, that the drawing(s) is for the purpose of illustration and description only and are not intended to limit the scope of the present disclosure. It is understood that the drawings are not to scale.

### Definitions

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “decreased,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, an “epitope” refers to the amino acids conventionally bound by an immunoglobulin VH/VL pair, such as the antibodies, antigen binding portions thereof and other binding agents described herein. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody, antigen binding portions thereof and other binding agent, and thus represents the target of specificity of an antibody, antigen binding portion thereof or other immunoglobulin-based binding agent. In the case of a single domain antibody, an epitope represents the unit of structure bound by a variable domain in isolation.

As used herein, “specifically binds” refers to the ability of a binding agent (e.g., an antibody or antigen binding portion thereof) described herein to bind to a target, such as human EGFR and/or c-MET, with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the antibody, antigen binding portion or other binding agent and the concentration of target polypeptide. The person of ordinary skill in the art can determine appropriate conditions under which the antibodies, antigen binding portions and other binding agents described herein selectively bind to EGFR and/or c-MET using any suitable methods, such as titration of an antibody or other binding agent in a suitable cell binding assay. A binding agent specifically bound to EGFR and/or c-MET is not displaced by a non-similar competitor. In certain embodiments, an EGFR and/or c-MET antibody or antigen-binding portion thereof or other binding agent is said to specifically bind to EGFR and/or c-MET when it preferentially recognizes its target antigen, EGFR and/or c-MET, in a complex mixture of proteins and/or macromolecules.

In some embodiments, an EGFR and/or c-MET antibody or antigen-binding portion thereof or other binding agent as described herein specifically binds to an EGFR and/or c-MET polypeptide with a dissociation constant (KD) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, an EGFR and/or c-MET antibody or antigen-binding portion thereof or other binding agent as described herein specifically binds to an EGFR and/or c-MET polypeptide with a dissociation constant (KD) of from about 10⁻⁵ M to 10⁻⁶ M. In some embodiments, an EGFR and/or c-MET antibody or antigen-binding portion thereof or other binding agent as described herein specifically binds to an EGFR and/or c-MET polypeptide with a dissociation constant (KD) of from about 10⁻⁷ M to 10⁻⁸ M. In some embodiments, an EGFR and/or c-MET antibody or antigen-binding portion thereof or other binding agent as described herein specifically binds to an EGFR and/or c-MET polypeptide with a dissociation constant (KD) of from about 10⁻⁸ M to 10⁻⁹ M. In some embodiments, an EGFR and/or c-MET antibody or antigen-binding portion thereof or other binding agent as described herein specifically binds to an EGFR and/or c-MET polypeptide with a dissociation constant (KD) of from about 10⁻⁹ M to 10⁻¹⁰ M. In some embodiments, an EGFR and/or c-MET antibody or antigen-binding portion thereof or other binding agent as described herein specifically binds to an EGFR and/or c-MET polypeptide with a dissociation constant (KD) of from about 10⁻¹⁰ M to 10⁻¹¹ M. In some embodiments, an EGFR and/or c-MET antibody or antigen-binding portion thereof or other binding agent as described herein specifically binds to an EGFR and/or c-MET polypeptide with a dissociation constant (KD) of from about 10⁻¹¹ M to 10⁻¹² M. In some embodiments, an EGFR and/or c-MET antibody or antigen-binding portion thereof or other binding agent as described herein specifically binds to an EGFR and/or c-MET polypeptide with a dissociation constant (KD) of from about 10⁻¹² M to 10⁻¹³ M. In some embodiments, an EGFR and/or c-MET antibody or antigen-binding portion thereof or other binding agent as described herein specifically binds to an EGFR and/or c-MET polypeptide with a dissociation constant (KD) of less than 10⁻⁹ M.

Unless otherwise indicated, the term “alkyl” by itself or as part of another term refers to a substituted or unsubstituted straight chain or branched, saturated hydrocarbon having the indicated number of carbon atoms (e.g., “—C1-C5 alkyl”, “—C1-C8 alkyl” or “—C1-C10” alkyl refer to an alkyl group having from 1 to 5, 1 to 8, or 1 to 10 carbon atoms, respectively). Examples include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

Unless otherwise indicated, “alkenyl” by itself or as part of another term refers to a C₂-C₈ substituted or unsubstituted straight chain or branched, hydrocarbon with at least one site of unsaturation (i.e., a carbon-carbon, sp2 double bond). Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

Unless otherwise indicated, “alkynyl” by itself or as part of another term refers to a refers to C2-C8, substituted or unsubstituted straight chain or branched, hydrocarbon with at least one site of unsaturation (i.e., a carbon-carbon, sp triple bond. Examples include, but are not limited to: acetylenic and propargyl.

Unless other indicated, “alkylene” refers to a saturated, branched or straight chain or hydrocarbon radical of 1-8 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

Unless otherwise indicated, “alkenylene” refers to an unsaturated, branched or straight chain hydrocarbon radical of 2-8 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

Unless otherwise indicated, “alkynylene” refers to an unsaturated, branched or straight chain or cyclic hydrocarbon radical of 2-8 carbon atoms, and having two monovalent radical centers derived by the removal of two hydrogen atoms from the same or two different carbon atoms of a parent alkyne. Typical alkynylene radicals include, but are not limited to: acetylene, propargyl, and 4-pentynyl.

Unless otherwise indicated, the term “heteroalkyl,” by itself or in combination with another term, refers to a substituted or unsubstituted stable straight or branched chain hydrocarbon, or combinations thereof, saturated and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group (i.e., as part of the main chain) or at the position at which the alkyl group is attached to the remainder of the molecule. The heteroatom Si may be placed at any position of the heteroalkyl group, including the position at which the alkyl group is attached to the remainder of the molecule. Examples of heteroalkyl include the following: —CH₂CH₂OCH₃, —CH₂CH₂NHCH₃, —CH₂CH₂N(CH₃)CH₃, —CH₂SCH₂CH₃, CH₂CH₂S(O)CH₃, —CH₂CH₂S(O)₂CH₃, and —Si(CH₃)₃, —. Up to two heteroatoms may be consecutive, such as, for example, —CH₂NHOCH₃ and CH₂OSi(CH₃)₃. In some embodiments, a C1 to C4 heteroalkyl has 1 to 4 carbon atoms and 1 or 2 heteroatoms and a C1 to C3 heteroalkyl has 1 to 3 carbon atoms and 1 or 2 heteroatoms.

Unless otherwise indicated, the terms “heteroalkenyl” and “heteroalkynyl” by themselves or in combination with another term, refers to a substituted or unsubstituted stable straight or branched chain alkenyl or alkynyl having from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of a heteroalkenyl or heteroalkynyl group (i.e., as part of the main chain) or at the position at which the alkyl group is attached to the remainder of the molecule. The heteroatom Si may be placed at any position of a heteroalkenyl or heteroalkynyl group, including the position at which the alkyl group is attached to the remainder of the molecule.

Unless otherwise indicated, the term “heteroalkylene” by itself or as part of another substituent refers to a substituted or unsubstituted divalent group derived from a heteroalkyl (as discussed above), as exemplified by —CH2CH2SCH2CH₂— and —CH2SCH2CH2NHCH2-. In some embodiments, a C1 to C4 heteroalkylene has 1 to 4 carbon atoms and 1 or 2 heteroatoms and a C1 to C3 heteroalkylene has 1 to 3 carbon atoms and 1 or 2 heteroatoms. For heteroalkylene groups, heteroatoms can also occupy either or both of the chain termini. Still further, for alkylene and heteroalkylene linking groups, no orientation of the linking group is implied.

Unless otherwise indicated, the terms “heteroalkenylene” and “heteroalkynylene” by themselves or as part of another substituent refers to a substituted or unsubstituted divalent group derived from an heteroalkenyl or heteroalkynyl (as discussed above). In some embodiments, a C2 to C4 heteroalkenylene or heteroalkynylene has 1 to 4 carbon atoms. For heteroalkenylene and heteroalkynylene groups, heteroatoms can also occupy either or both of the chain termini. Still further, for alkylene and heteroalkenylene and heteroalkynylene linking groups, no orientation of the linking group is implied.

Unless otherwise indicated, a “C3-C8 carbocycle,” by itself or as part of another term, refers to a substituted or unsubstituted 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C3-C8 carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C3-C8 carbocyclo”, by itself or as part of another term, refers to a substituted or unsubstituted C3-C8 carbocycle group defined above wherein another of the carbocycle groups' hydrogen atoms is replaced with a bond (i.e., it is divalent).

Unless otherwise indicated, a “C3-C10 carbocycle,” by itself or as part of another term, refers to a substituted or unsubstituted 3-, 4-, 5-, 6-, 7-, 8-, 9- or 10-membered monovalent, substituted or unsubstituted, saturated or unsaturated non-aromatic monocyclic, bicyclic or tricyclic carbocyclic ring derived by the removal of one hydrogen atom from a ring atom of a parent ring system. Representative —C3-C10 carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. —C3-C10 carbocycles can further include fused cyclooctyne carbocycles, such as the fused cyclooctyne compounds disclosed in International Publication Number WO2011/136645 (the disclosure of which is incorporated by reference herein), including BCN (bicyclo[6.1.0]nonyne) and DBCO (Dibenzocyclooctyne).

Unless otherwise indicated, a “C3-C8 heterocycle,” by itself or as part of another term, refers to a substituted or unsubstituted monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a C3-C8 heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. Unless otherwise indicate, the term “heterocarbocycle” is synonymous with the terms “heterocycle” or “heterocyclo” as described herein.

Unless otherwise indicated, “C3-C8 heterocyclo”, by itself or as part of another term, refers to a substituted or unsubstituted C3-C8 heterocycle group defined above wherein one of the heterocycle group's hydrogen atoms is replaced with a bond (i.e., it is divalent).

Unless otherwise indicated, “aryl” by itself or as part of another term, means a substituted or unsubstituted monovalent carbocyclic aromatic hydrocarbon radical of 6-20 carbon (preferably 6-14 carbon) atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene” by itself or as part of another term, is an unsubstituted or substituted aryl group as defined above wherein one of the aryl group's hydrogen atoms is replaced with a bond (i.e., it is divalent) and can be in the ortho, meta, or para orientations.

Unless otherwise indicated, “heteroaryl” and “heterocycle” refer to a ring system in which one or more ring atoms is a heteroatom, e.g., nitrogen, oxygen, and sulfur. A heterocycle radical comprises 1 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system.

Unless otherwise indicated, an “heteroarylene” by itself or as part of another term, is an unsubstituted or substituted heteroaryl group as defined above wherein one of the heteroaryl group's hydrogen atoms is replaced with a bond (i.e., it is divalent).

Unless otherwise indicated, “carboxyl” refers to COOH or COO-M⁺, where M⁺ is a cation.

Unless otherwise indicated, “oxo” refers to (C═O).

Unless otherwise indicated, “substituted alkyl” and “substituted aryl” mean alkyl and aryl, respectively, in which one or more hydrogen atoms are each independently replaced with a substituent. Typical substituents include, but are not limited to, —X, —R10, —O—, —OR10, —SR10, —S—, —NR102, —NR103, ═NR10, —CX3, —CN, —OCN, —SCN, —N═C═O, —NCS, —NO, —NO2, ═N2, —N3, —NR10C(═O)R10, —C(═O)R10, —C(═O)NR102, —SO3-, —SO3H, —S(═O)2R10, —OS(═O)2OR10, —S(═O)2NR10, —S(═O)R10, —OP(═O)(OR10)2, —P(═O)(OR10)2, —PO-3, —PO3H2, —AsO2H2, —C(═O)R10, —C(═O)X, —C(═S)R10, —CO2R10, —CO2-, —C(═S)OR10, C(═O)SR10, C(═S)SR10, C(═O)NR102, C(═S)NR102, or C(═NR10)NR102, where each X is independently a halogen: —F, —C, —Br, or —I; and each R10 is independently —H, —C₁-C₂₀ alkyl, —C₆-C₂₀ aryl, —C₃-C₁₄ heterocycle, a protecting group or a prodrug moiety. Typical substitutents also include (═O). Alkylene, carbocycle, carbocyclo, arylene, heteroalkyl, heteroalkylene, heterocycle, and heterocyclo groups as described above may also be similarly substituted.

Unless otherwise indicated, “polyhydroxyl group” refers to an alkyl, alkylene, carbocycle or carbocyclo group including two or more, or three or more, substitutions of hydroxyl groups for hydrogen on carbon atoms of the carbon chain. In some embodiments, a polyhydroxyl group comprises at least three hydroxyl groups. In some embodiments, a polyhydroxyl group comprises carbon atoms containing only one hydroxyl group per carbon atom. A polyhydroxyl group may contain one or more carbon atoms that are not substituted with hydroxyl. A polyhydroxyl group may have each carbon atom substituted with a hydroxyl group. Examples of polyhydroxyl group includes linear (acyclic) or cyclic forms of monosaccharides such as C6 or C5 sugars, such as glucose, ribose, galactose, mannose, arabinose, 2-deoxyglucose, glyceraldehyde, erythrose, threose, xylose, lyxose, allose, altrose, gulose, idose, talose, aldose, and ketose, sugar acids such as gluconic acid, aldonic acid, uronic acid or ulosonic acid, and an amino sugars, such as glucosamine, N-acetyl glucosamine, galactosamine, and N-acetyl galactosamine. In some embodiments, polyhydroxyl group includes linear or cyclic forms of disaccharides and polysaccharides.

Unless otherwise indicated by context, “optionally substituted” refers to an alkyl, alkenyl, alkynyl, alkylaryl, arylalkyl heterocycle, aryl, heteroaryl, alkylheteroaryl, heteroarylalkyl, or other substituent, moiety or group as defined or disclosed herein wherein hydrogen atom(s) of that substituent, moiety or group has been optionally replaced with different moiety(ies) or group(s), or wherein an alicyclic carbon chain that comprise one of those substituents, moiety or group is interrupted by replacing carbon atom(s) of that chain with different moiety(ies) or group(s). In some aspects an alkene function group replaces two contiguous sp3 carbon atoms of an alkyl substituent, provided that the radical carbon of the alkyl moiety is not replaced, so that the optionally substituted alkyl is an unsaturated alkyl substituent.

Optional substituent replacing hydrogen(s) in any of the foregoing substituents, moieties or groups is independently selected from the group consisting of aryl, heteroaryl, hydroxyl, alkoxy, aryloxy, cyano, halogen, nitro, fluoroalkoxy, and amino, including mono-, di- and tri-substituted amino groups, and the protected derivatives thereof, or is selected from the group consisting of —X, —OR′, —SR′, —NH2, —N(R′)(R″), —N(R″)3, ═NR, —CX3, —CN, —NO2, —NR′C(═O)H, —NR′C(═O)R, —NR′C(═O)R″, —C(═O)R′, —C(═O)NH2, —C(═O)N(R′)R″, —S(═O)2R″, —S(═O)2NH2, —S(═O)2N(R′)R″, —S(═O)2NH2, —S(═O)2N(R′)R″, —S(═O)2OR′, —S(═O)R″, —OP(═O)(OR′)(OR″), —OP(OH)3, —P(═O)(OR′)(OR″), —PO3H2, —C(═O)R′, —C(═S)R″, —CO2R′, —C(═S)OR″, —C(═O)SR′, —C(═S)SR′, —C(═S)NH2, —C(═S)N(R′)(R″)2, —C(═NR′)NH2, —C(═NR′)N(R′)R″, and salts thereof, wherein each X is independently selected from the group consisting of a halogen: —F, —Cl, —Br, and —I; and wherein each R″ is independently selected from the group consisting of C1-C20 alkyl, C2-C20 alkenyl, C2-C20 alkynyl, C6-C24 aryl, C3-C24 heterocyclyl (including C5-C24 heteroaryl), a protecting group, and a prodrug moiety or two of R″ together with the heteroatom to which they are attached defines a heterocyclyl; and R′ is hydrogen or R″, wherein R″ is selected from the group consisting of C1-C20 alkyl, C6-C24 aryl, C3-C24 heterocyclyl (including C5-C24 heteroaryl), and a protecting group.

Typically, optional substituents are selected from the group consisting of —X, —OH, —OR″, —SH, —SR″, —NH2, —NH(R″), —NR′(R″)2, —N(R″)3, =NH, =NR″, —CX3, —CN, —NO2, —NR′C(═O)H, NR′C(═O)R″, —CO2H, —C(═O)H, —C(═O)R″, —C(═O)NH2, —C(═O)NR′R″— —S(═O)2R″, —S(═O)2NH2, —S(═O)2N(R′)R″, —S(═O)2NH2, —S(═O)2N(R′)(R″), —S(═O)2OR′, —S(═O)R″, —C(═S)R″, —C(═S)NH2, —C(═S)N(R′)R″, —C(═NR′)N(R″)2, and salts thereof, wherein each X is independently selected from the group consisting of —F and —Cl, R″ is typically selected from the group consisting of C1-C6 alkyl, C6-C10 aryl, C3-C10 heterocyclyl (including C5-C10 heteroaryl), and a protecting group; and R′ independently is hydrogen, C1-C6 alkyl, C6-C10 aryl, C3-C10 heterocyclyl (including C5-C10 heteroaryl), and a protecting group, independently selected from R″. More typically, substituents are selected from the group consisting of —X, —R″, —OH, —OR″, —NH2, —NH(R″), —N(R″)2, —N(R″)3, —CX3, —NO2, —NHC(═O)H, —NHC(═O)R″, —C(═O)NH2, —C(═O)NHR″, —C(═O)N(R″)2, —CO2H, —CO2R″, —C(═O)H, —C(═O)R″, —C(═O)NH2, —C(═O)NH(R″), —C(═O)N(R″)2, —C(═NR′)NH2, —C(═NR′)NH(R″), —C(═NR′)N(R″)2, a protecting group and salts thereof, wherein each X is —F, R″ is independently selected from the group consisting of C1-C6 alkyl, C6-C10 aryl, C5-C10 heteroaryl and a protecting group; and R′ is selected from the group consisting of hydrogen, C1-C6 alkyl and a protecting group, independently selected from R″.

The compounds of the invention, or their pharmaceutically acceptable salts may contain one or more asymmetric centers and may thus give rise to enantiomers, diastereomers, and other stereoisomeric forms that are defined, in terms of absolute stereochemistry, as (R) or (S) or, as (D) or (L) for amino acids. The present invention is meant to include all such possible isomers, as well as their racemic and optically pure forms. Optically active (+) and (−), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another. The present invention also includes “diastereomers”, which refers to two or more stereoisomers of a compound that have different configurations at one or more of the equivalent stereocenters and are not mirror images of each other.

Although structures shown throughout the specification are depicted with specific stereocenters, the specification should be read to include variations in those stereocenters. For example, the structure of exatecan may be shown in the (S,S) configuration, but the (R,S) diastereomer of exatecan is also envisioned as being found in a separate embodiment of a conjugate as described herein.

Unless otherwise indicated, the term “drug unit” or drug refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

Unless otherwise indicated, the term “polymer unit” refers to a polymeric moiety composed of repeating subunits. Examples of polymer units include polyamides and polyethers. In some embodiments, the polymer unit is selected from an optionally substituted polyamide, a substituted polyether, or combinations thereof. In further embodiments, the polymer unit is selected from
(i) an optionally substituted polyamide comprising the formula

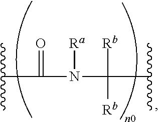

or a stereoisomer thereof, wherein each Ra is independently H or C1-6 alkyl and each Rb is independently H or C1-6 alkyl, and n0 is independently 2-26;
(ii) a substituted polyether comprising the formula

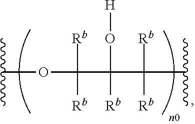

or a stereoisomer thereof, wherein each Rb is independently H or C1-6 alkyl, and n0 is independently 2-26; or
(iii) combinations thereof.

Unless otherwise indicated, the term “sugar unit” or “sugar group” refers to a carbohydrate group. Examples of sugar units include glycosides.

Unless otherwise indicated, the term “carboxyl unit” or “carboxyl group” refers to a group including a carbonyl group [—C(O)—], a carboxyl group [—CO2H], and/or a carboxylate group [—CO2M, M refers to a cationic counterion].

Unless otherwise indicated, the term “stretcher group” refers to a linking moiety that connects the EGFR and/or c-MET binding agent to the enzyme-cleavable group.

Unless otherwise indicated, the term “polyamide” refers to polymeric groups composed of repeating subunits containing amide bonds.

Unless otherwise indicated, the term “polyether” refers to polymeric groups composed to repeating subunits containing ether bonds.

Unless otherwise indicated, the term “enzyme-cleavable group” refers to a group that is cleavable by the action of a metabolic process or reaction inside a cell or in the extracellular milieu, whereby the covalent attachment between a drug unit (e.g., a cytotoxic agent) and the linker unit or portion thereof is broken, resulting in the free drug unit, or a metabolite of the linker unit-drug, which is dissociated from the remainder of the linker unit.

The phrase “pharmaceutically acceptable salt,” as used herein, refers to pharmaceutically acceptable organic or inorganic salts of a compound (e.g., a linker, drug linker, or a conjugate). The compound typically contains at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, linleate, gentisinate, fumarate, gluconate, glucuronate, saccharate, formate, benzoate, glutamate, methanesulfonate, ethanesulfonate, benzenesulfonate, toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

As used herein, the term “consisting essentially of” refers to those elements required for a given embodiment. The term permits the presence of elements that do not materially affect the basic and novel or functional characteristic(s) of that embodiment.

As used herein, the term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean +/−1%.

The terms “statistically significant” or “significantly” refer to statistical significance and generally mean a two standard deviation (2SD) difference, above or below a reference value.

Other terms are defined herein within the description of the various aspects of the invention.

### Antibodies and Binding Agents

Provided herein are EGFR and/or c-MET binding antibodies (also referred to as EGFR and/or c-MET antibodies) and antigen binding portions thereof and other binding agents that specifically bind to human EGFR and/or c-MET. Also provided herein are conjugates of the EGFR and/or c-MET antibodies and antigen binding portions and other binding agents bound to drugs, such as cytotoxic agents or immune modulatory agents (also referred to as EGFR and/or c-MET conjugates). In some embodiments, the EGFR and/or c-MET antibodies, antigen binding portions, other binding agents and/or EGFR and/or c-MET conjugates specifically bind to and reduce the number of EGFR and/or c-MET+ cells in a subject. In some embodiments, the EGFR and/or c-MET antibodies, antigen binding portions, other binding agents and/or EGFR and/or c-MET conjugates specifically bind to and reduce the number of EGFR and/or c-MET+ cancer cells in a subject. In some embodiments, the EGFR and/or c-MET antibodies, antigen binding portions, other binding agents and/or EGFR and/or c-MET conjugates specifically bind to and reduce the number of EGFR and/or c-MET+ cells associated with a disease or condition in a subject, such as a cancer or an autoimmune disease. In some embodiments, the EGFR and/or c-MET antibodies, antigen binding portions, other binding agents and/or EGFR and/or c-MET conjugates specifically bind to and reduce the number of EGFR and/or c-MET+ cells associated with a disease or condition in a subject, such as a human or an animal.

In some embodiments, a bispecific binding agent comprising a first binding domain that binds to EGFR and a second binding domain that binds to c-MET. The first binding domain that binds to EGFR comprises a heavy chain comprising a heavy chain variable (VH) region and a light chain comprising a light chain variable (VL) region. The VH region comprises complementarity determining regions HCDR1, HCDR2, and HCDR3 disposed in heavy chain variable region framework regions, and the VL region comprises LCDR1, LCDR2, and LCDR3 disposed in light chain variable region framework regions. The HCDR1 of the first binding domain has an amino acid sequence of SEQ ID NO: 139 or 174, the HCDR2 of the first binding domain has an amino acid sequence of SEQ ID NO: 140 or 175, the HCDR3 of the first binding domain has an amino acid sequence of SEQ ID NO: 141 or 176, the LCDR1 of the first binding domain has an amino acid sequence of SEQ ID NO: 142 or 177, the LCDR2 of the first binding domain has an amino acid sequence of DAS or KVS, and the LCDR3 of the first binding domain has an amino acid sequence of SEQ ID NO: 143 or 178.

In some embodiments, the VH and VL regions of the first binding domain that binds to EGFR have amino acid sequences that are selected from the pairs of amino acid sequences set forth in the group consisting of: SEQ ID NO: 137 and SEQ ID NO: 138, respectively; SEQ ID NO: 157 and SEQ ID NO: 158, respectively; SEQ ID NO: 172 and SEQ ID NO: 173, respectively; SEQ ID NO: 187 and SEQ ID NO: 188, respectively; SEQ ID NO: 198 and SEQ ID NO: 199, respectively; SEQ ID NO: 208 and SEQ ID NO: 209, respectively; SEQ ID NO: 218 and SEQ ID NO: 219, respectively; SEQ ID NO: 228 and SEQ ID NO: 229, respectively; SEQ ID NO: 243 and SEQ ID NO: 244, respectively; SEQ ID NO: 251 and SEQ ID NO: 252, respectively; and SEQ ID NO: 259 and SEQ ID NO: 260, respectively.

In some embodiments, the second binding domain comprises a heavy chain and a light chain, the heavy chain comprising a heavy chain variable (VH) region and the light chain comprising a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, wherein the HCDR1 of the second binding domain has an amino acid sequence of SEQ ID NO: 149, 164, 194, or 235, the HCDR2 of the second binding domain has an amino acid sequence of SEQ ID NO: 150, 165, or 236, the HCDR3 of the second binding domain has an amino acid sequence of SEQ ID NO: 151,166, or 237, the LCDR1 of the second binding domain has an amino acid sequence of SEQ ID NO: 152, 167, or 238, the LCDR2 of the second binding domain has an amino acid sequence of RAS, WAS, or AAS, and the LCDR3 of the second binding domain has an amino acid sequence of SEQ ID NO: 153, 168, or 239.

In some embodiments, the VH and VL regions of the second binding domain that binds to c-MET have amino acid sequences that are selected from the pairs of amino acid sequences set forth in the group consisting of: SEQ ID NO: 147 and SEQ ID NO: 148, respectively; SEQ ID NO: 162 and SEQ ID NO: 163, respectively; SEQ ID NO: 182 and SEQ ID NO: 183, respectively; SEQ ID NO: 192 and SEQ ID NO: 193, respectively; SEQ ID NO: 203 and SEQ ID NO: 204, respectively; SEQ ID NO: 213 and SEQ ID NO: 214, respectively; SEQ ID NO: 223 and SEQ ID NO: 224, respectively; SEQ ID NO: 233 and SEQ ID NO: 234, respectively; SEQ ID NO: 248 and SEQ ID NO: 249, respectively; SEQ ID NO: 254 and SEQ ID NO: 255, respectively; and SEQ ID NO: 264 and SEQ ID NO: 265, respectively.

In some embodiments, the framework regions are human framework regions. As used herein, the term “antibody” refers to immunoglobulin molecules and immunologically active portions of immunoglobulin molecules, i.e., molecules that contain an antigen binding site(s) that specifically binds to an antigen, e.g., human EGFR or c-MET. The term generally refers to antibodies comprised of two immunoglobulin heavy chain variable regions and two immunoglobulin light chain variable regions including full length antibodies (having heavy and light chain constant regions).

Each heavy chain is composed of a variable region (abbreviated as VH) and a constant region. The heavy chain constant region may include three domains CH1, CH2 and CH3 and optionally a fourth domain, CH4. Each light chain is composed of a variable region (abbreviated as VL) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs that are arranged from the N terminus to the C terminus in the following order: FR1, CDR1, FR2, CDR2, FR3, CDR3, and FR4. This structure is well known to those skilled in the art.

As used herein, an “antigen-binding portion” of EGFR and c-MET bispecific antibodies refers to the portions of EGFR and c-MET bispecific antibodies as described herein having the VH and VL sequences of the EGFR and c-MET bispecific antibodies or the CDRs of EGFR and c-MET bispecific antibodies and that specifically binds to EGFR and c-MET. Examples of antigen binding portions include a Fab, a Fab′, an F(ab′), an Fv, a scFv, a disulfide linked Fv, a single domain antibody (also referred to as a VHH, VNAR, sdAb, or nanobody) or a diabody (see, e.g., Huston et al., Proc. Natl. Acad. Sci. U.S.A., 85, 5879-5883 (1988) and Bird et al., Science 242, 423-426 (1988), which are incorporated herein by reference). As used herein, the terms Fab, F(ab′) and Fv refer to the following: (i) a Fab fragment, i.e. a monovalent fragment composed of the VL, VH, CL and CH1 domains; (ii) an F(ab′) fragment, i.e. a bivalent fragment comprising two Fab fragments linked to one another in the hinge region via a disulfide bridge; and (iii) an Fv fragment composed of the VL and VH domains, in each case of EGFR and c-MET bispecific antibodies. Although the two domains of the Fv fragment, namely VL and VH, are encoded by separate coding regions, they may further be linked to one another using a synthetic linker, e.g., a poly-G4S amino acid sequence (‘(G4S)n’ disclosed as SEQ ID NO: 58, wherein n=1 to 5), making it possible to prepare them as a single protein chain in which the VL and VH regions combine in order to form monovalent molecules (known as single chain Fv or scFv). The term “antigen-binding portion” of an antibody is also intended to include such single chain antibodies. Other forms of single chain antibodies such as “diabodies” are likewise included here. Diabodies are bivalent, bispecific antibodies in which VH and VL domains are expressed on a single polypeptide chain, but using a linker connecting the VH and VL domains that is too short for the two domains to be able to combine on the same chain, thereby forcing the VH and VL domains to pair with complementary domains of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

A single-domain antibody is an antibody portion consisting of a single monomeric variable antibody domain. Single domains antibodies can be derived from the variable domain of the antibody heavy chain from camelids (e.g., nanobodies or VHH portions). Furthermore, the term single-domain antibody includes an autonomous human heavy chain variable domain (aVH) or VNAR portions derived from sharks (see, e.g., Hasler et al., Mol. Immunol. 75:28-37, 2016).

Techniques for producing single domain antibodies (e.g., DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with novel epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize a target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

In some embodiments, the EGFR and c-MET bispecific antibodies or antigen binding portions thereof are part of a bispecific or multispecific binding agent. Bispecific and multi-specific antibodies include the following: an scFv1-ScFv2, an ScFv12-Fc-scFv22, an IgG-scFv, a DVD-Ig, a triomab/quadroma, a two-in-one IgG, a scFv2-Fc, a TandAb, and an scFv-HSA-scFv. In some embodiments, an IgG-scFv is an IgG(H)-scFv, scFv-(H)IgG, IgG(L)-scFv, svFc-(L)IgG, 2scFV-IgG or IgG-2scFv. See, e.g., Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

### Modification of VH and VL Regions

As to the VH and VL amino acid sequences, one of skill will recognize that individual substitutions, deletions or additions (insertions) to a nucleic acid encoding the VH or VL, or amino acids in a polypeptide that alter a single amino acid or a small percentage of amino acids in the encoded sequence is a “conservatively modified variant”, where the alteration results in the substitution of an amino acid with a chemically similar amino acid (a conservative amino acid substitution) and the altered polypeptide retains the ability to specifically bind to EGFR and/or c-MET.

In some embodiments, a conservatively modified variant of EGFR and c-MET bispecific antibodies or antigen binding portion thereof can have an alteration(s) in the framework regions (i.e., other than in the CDRs), e.g. a conservatively modified variant of EGFR and c-MET bispecific antibodies having the amino acid sequences of the VH and VL CDRs and has at least one conservative amino acid substitution in a framework region (FR). In some embodiments, the VH and VL amino acid sequences collectively have no more than 8 or 6 or 4 or 2 or 1 conservative amino acid substitutions in the FR, as compared to the amino acid sequences of the unmodified VH and VL regions. In some embodiments, the VH and VL amino acid sequences have 8 to 1, 6 to 1, 4 to 1 or 2 to 1 conservative amino acid substitutions in the FR, as compared to the amino acid sequences of the unmodified VH and VL regions. In further aspects of any of these embodiments, a conservatively modified variant of the EGFR and c-MET bispecific antibodies, antigen binding portion thereof or other binding agent exhibits specific binding to EGFR and/or c-MET.

For conservative amino acid substitutions, a given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gln and Asn). Other such conservative amino acid substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained, i.e., to EGFR and/or c-MET.

In some embodiments, EGFR and c-MET bispecific antibodies or antigen binding portion thereof or other binding agent can be further optimized to, for example, decrease potential immunogenicity or optimize other functional property, while maintaining functional activity, for therapy in humans.

In some embodiments, the EGFR and c-MET bispecific antibodies or antigen binding portions thereof or other binding agents comprise a first binding domain that binds to EGFR and a second binding domain that binds to c-MET. The first binding domain that binds to EGFR comprises a heavy chain comprising a heavy chain variable (VH) region and a light chain comprising a light chain variable (VL) region. The VH and VL regions of the first binding domain that binds to EGFR have amino acid sequences that are selected from the pairs of amino acid sequences set forth in the group consisting of: SEQ ID NO: 137 and SEQ ID NO: 138, respectively; SEQ ID NO: 157 and SEQ ID NO: 158, respectively; SEQ ID NO: 172 and SEQ ID NO: 173, respectively; SEQ ID NO: 187 and SEQ ID NO: 188, respectively; SEQ ID NO: 198 and SEQ ID NO: 199, respectively; SEQ ID NO: 208 and SEQ ID NO: 209, respectively; SEQ ID NO: 218 and SEQ ID NO: 219, respectively; SEQ ID NO: 228 and SEQ ID NO: 229, respectively; SEQ ID NO: 243 and SEQ ID NO: 244, respectively; SEQ ID NO: 251 and SEQ ID NO: 252, respectively; and SEQ ID NO: 259 and SEQ ID NO: 260, respectively, wherein the heavy and light chain framework regions are optionally modified with from 1 to 8 amino acid substitutions, deletions or insertions in the framework regions.

In some embodiments, a bispecific binding agent comprising: a first binding domain that binds to EGFR; and a second binding domain that binds to c-MET, wherein the first binding domain comprises a heavy chain and a light chain, the heavy chain comprising a heavy chain variable (VH) region and the light chain comprising a light chain variable (VL) region, the VH and VL regions of the second binding domain that binds to c-MET have amino acid sequences that are selected from the pairs of amino acid sequences set forth in the group consisting of: SEQ ID NO: 147 and SEQ ID NO: 148, respectively; SEQ ID NO: 162 and SEQ ID NO: 163, respectively; SEQ ID NO: 182 and SEQ ID NO: 183, respectively; SEQ ID NO: 192 and SEQ ID NO: 193, respectively; SEQ ID NO: 203 and SEQ ID NO: 204, respectively; SEQ ID NO: 213 and SEQ ID NO: 214, respectively; SEQ ID NO: 223 and SEQ ID NO: 224, respectively; SEQ ID NO: 233 and SEQ ID NO: 234, respectively; SEQ ID NO: 248 and SEQ ID NO: 249, respectively; SEQ ID NO: 254 and SEQ ID NO: 255, respectively; and SEQ ID NO: 264 and SEQ ID NO: 265, respectively, wherein the heavy and light chain framework regions are optionally modified with from 1 to 8 amino acid substitutions, deletions or insertions in the framework regions.

In any of these embodiments, the functional activity of the EGFR and C-MET bispecific binding antibody or antigen binding portion thereof or other binding agent includes specifically binding to EGFR and/or c-MET. Additional functional activities include depletion of EGFR and/or c-MET cells (e.g., cancer cells or autoimmune cells). In the case where dose dependency does exist, it needs not be identical to that of the reference antibody or antigen-binding portion thereof, but rather substantially similar to or better than the dose-dependence in a given activity as compared to the reference antibody or antigen-binding portion thereof as described herein (i.e., the candidate polypeptide will exhibit greater activity relative to the reference antibody).

For conservative substitutions, amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, for conservative substitutions naturally occurring residues can be divided into groups based on common side-chain properties: (1) hydrophobic: Norleucine, Met, Ala, Val, Leu, lie; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes or another class.

Particular conservative substitutions include, for example; Ala to Gly or to Ser; Arg to Lys; Asn to Gln or to His; Asp to Glu; Cys to Ser; Gln to Asn; Glu to Asp; Gly to Ala or to Pro; His to Asn or to Gln; Ile to Leu or to Val; Leu to Ile or to Val; Lys to Arg, to Gln or to Glu; Met to Leu, to Tyr or to lie; Phe to Met, to Leu or to Tyr; Ser to Thr; Thr to Ser; Trp to Tyr; Tyr to Trp; and/or Phe to Val, to Ile or to Leu.

In some embodiments, a conservatively modified variant of an EGFR and c-MET antibody or antigen binding portion thereof preferably is at least 90%, at least 91%, at least 92%, at least 93%, at least 94%, at least 95%, at least 96%, at least 97%, at least 98%, at least 99%, or more, identical to the reference VH or VL sequence, wherein the VH and VL CDRs are not modified. The degree of homology (percent identity) between the reference and modified sequence can be determined, for example, by comparing the two sequences using freely available computer programs commonly employed for this purpose on the world wide web (e.g., BLASTp or BLASTn with default settings).

In some embodiments, the VH and VL amino acid sequences collectively have no more than 8 or 6 or 4 or 2 or 1 conservative amino acid substitutions in the framework regions, as compared to the amino acid sequences of the unmodified VH and VL regions. In some embodiments, the VH and VL amino acid sequences collectively have 8 to 1, or 6 to 1, or 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions in the framework regions, as compared to the amino acid sequences of the unmodified VH and VL regions. In some embodiments, the VH and VL amino acid sequences have 8 to 1, 6 to 1, 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions, as compared to the amino acid sequences of the unmodified VH and VL regions.

Modification of a native (or reference) amino acid sequence can be accomplished by any of a number of techniques known to one of skill in the art. Mutations can be introduced, for example, at particular loci by synthesizing oligonucleotides containing the desired mutant sequence, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion desired. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

### Constant Regions

In some embodiments, the EGFR and c-MET bispecific antibodies or antigen-binding portion thereof or other binding agent has fully human constant regions. In some embodiments, the EGFR and c-MET bispecific antibodies or antigen-binding portion thereof or other binding agent has humanized constant regions. In some embodiments, the EGFR and c-MET bispecific antibodies or antigen-binding portion thereof or other binding agent has non-human constant regions. An immunoglobulin constant region refers to a heavy or light chain constant region. Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgG1, IgG2, IgG3, IgG4, or IgAI, and IgA2. The heavy-chain constant regions (Fc) that correspond to the different classes of immunoglobulins can be α, δ, ε, γ, and μ, respectively. The light chains can be one of either kappa (or κ) and lambda (or λ).

In some embodiments, a constant region can have an IgG1 isotype. In some embodiments, a constant region can have an IgG2 isotype. In some embodiments, a constant region can have an IgG3 isotype. In some embodiments, a constant region can have an IgG4 isotype. In some embodiments, an Fc domain can have a hybrid isotype comprising constant regions from two or more isotypes. In some embodiments, an immunoglobulin constant region can be an IgG1 or IgG4 constant region. In some embodiments, the EGFR and c-MET bispecific antibodies heavy chain is of the IgG1 isotype and has the amino acid sequence set forth in SEQ ID NO: 266, SEQ ID NO:267, SEQ ID NO:268, SEQ ID NO:269, or SEQ ID NO:270. In some embodiments, the EGFR and c-MET bispecific antibodies light chain is of the kappa isotype and has the amino acid sequence set forth in SEQ ID NO:271.

Furthermore, the EGFR and c-MET bispecific antibodies or an antigen-binding portion thereof or other binding agent may be part of a larger binding agent formed by covalent or noncovalent association of the antibody or antigen binding portion with one or more other proteins or peptides. Relevant to such binding agents are the use, for example, of the streptavidin core region in order to prepare a tetrameric scFv molecule (Kipriyanov, S. M., et al. (1995), Human Antibodies and Hybridomas 6:93-101) and the use of a cysteine residue, a marker peptide and a C-terminal polyhistidinyl peptide, e.g. hexahistidinyl tag (‘hexahistidinyl tag’ disclosed as SEQ ID NO: 59) in order to produce bivalent and biotinylated scFv molecules (Kipriyanov, S. M., et al. (1994) Mol. Immunol. 31:10471058).

### Fc Domain Modifications to Alter Effector Function

In some embodiments, an Fc region or Fc domain of the EGFR and c-MET bispecific antibodies or antigen binding portion thereof or other binding agent has substantially no binding to at least one Fc receptor selected from FcγRI (CD64), FcγRIIA (CD32a), FcγRIIB (CD32b), FcγRIIIA (CD16a), and FcγRIIIB (CD16b). In some embodiments, an Fc region or domain exhibits substantially no binding to any of the Fc receptors selected from FcγRI (CD64), FcγRIIA (CD32a), FcγRIIB (CD32b), FcγRIIIA (CD16a), and FcγRIIIB (CD16b). As used herein, “substantially no binding” refers to weak to no binding to a selected Fcgamma receptor or receptors. In some embodiments, “substantially no binding” refers to a reduction in binding affinity (i.e., increase in Kd) to a Fc gamma receptor of at least 1000-fold. In some embodiments, an Fc domain or region is an Fc null. As used herein, an “Fc null” refers to an Fc region or Fc domain that exhibits weak to no binding to any of the Fcgamma receptors. In some embodiments, an Fc null domain or region exhibits a reduction in binding affinity (i.e., increase in Kd) to Fc gamma receptors of at least 1000-fold.

In some embodiments, an Fc domain has reduced or substantially no effector function activity. As used herein, “effector function activity” refers to antibody dependent cellular cytotoxicity (ADCC), antibody dependent cellular phagocytosis (ADCP) and/or complement dependent cytotoxicity (CDC). In some embodiments, an Fc domain exhibits reduced ADCC, ADCP or CDC activity, as compared to a wildtype Fc domain. In some embodiments, an Fc domain exhibits a reduction in ADCC, ADCP and CDC, as compared to a wildtype Fc domain. In some embodiments, an Fc domain exhibits substantially no effector function (i.e., the ability to stimulate or effect ADCC, ADCP or CDC). As used herein, “substantially no effector function” refers to a reduction in effector function activity of at least 1000-fold, as compared to a wildtype or reference Fc domain.

In some embodiments, an Fc domain has reduced or no ADCC activity. As used herein reduced or no ADCC activity refers to a decrease in ADCC activity of an Fc domain by a factor of at least 10, at least 20, at least 30, at least 50, at least 100 or at least 500.

In some embodiments, an Fc domain has reduced or no CDC activity. As used herein reduced or no CDC activity refers to a decrease in CDC activity of an Fc domain by of a factor of at least 10, at least 20, at least 30, at least 50, at least 100 or at least 500.

In vitro and/or in vivo cytotoxicity assays can be conducted to confirm the reduction/depletion of ADCC and/or CDC activity. For example, Fc receptor (FcR) binding assays can be conducted to ensure that the antibody lacks Fcγ receptor binding (hence likely lacking ADCC activity). The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that an antibody or Fc domain or region is unable to bind C1q and hence lacks CDC activity or has reduced CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)).

In some embodiments, an Fc domain has reduced or no ADCP activity. As used herein reduced or no ADCP activity refers to a decrease in ADCP activity of an Fc domain by a factor of at least 10, at least 20, at least 30, at least 50, at least 100 or at least 500.

ADCP binding assays may also be carried out to confirm that an antibody or Fc domain or region lacks ADCP activity or has reduced ADCP activity. See, e.g., US20190079077 and US20190048078 and the references disclosed therein.

The EGFR and c-MET bispecific antibodies or antigen binding portion thereof or other binding agent with reduced effector function activity includes those with substitution of one or more of Fc region residues, such as, for example, 238, 265, 269, 270, 297, 327 and 329, according to the EU number of Kabat (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine, according to the EU numbering of Kabat (see U.S. Pat. No. 7,332,581). Certain antibody variants with diminished binding to FcRs are also known. (See, e.g., U.S. Pat. No. 6,737,056; WO 2004/056312, and Shields et al., J. Biol. Chem. 9(2): 6591-6604 (2001).) The EGFR and c-MET bispecific antibodies or antigen binding portion thereof or other binding agent with diminished binding to FcRs can be prepared containing such amino acid modifications.

In some embodiments, the EGFR and c-MET bispecific antibodies or antigen binding portion thereof or other binding agent comprises an Fc domain or region with one or more amino acid substitutions which diminish FcγR binding, e.g., substitutions at positions 234 and 235 of the Fc region (EU numbering of residues). In some embodiments, the substitutions are L234A and L235A (LALA), according to the EU numbering of Kabat. In some embodiments, the Fc domain comprises D265A and/or P329G in an Fc region derived from a human IgG1 Fc region, according to the EU numbering of Kabat. In some embodiments, the substitutions are L234A, L235A and P329G (LALA-PG), according to the EU numbering of Kabat, in an Fc region derived from a human IgG1 Fc region. (See, e.g., WO 2012/130831). In some embodiments, the substitutions are L234A, L235A and D265A (LALA-DA) in an Fc region derived from a human IgG1 Fc region, according to the EU numbering of Kabat.

In some embodiments, alterations are made in the Fc region that result in altered (i.e., either diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

### Methods of Making Antibodies, Antigen Binding Portions and Other Binding Agents

In various embodiments, the EGFR and c-MET bispecific antibodies, antigen binding portions thereof and other binding agents can be produced in human, murine or other animal-derived cells lines. Recombinant DNA expression can be used to produce the EGFR and c-MET bispecific antibodies, antigen binding portions thereof and other binding agents. This allows the production of the EGFR and c-MET bispecific antibodies as well as a spectrum of EGFR and c-MET antigen binding portions and other binding agents (including fusion proteins) in a host species of choice. The production of the EGFR and c-MET bispecific antibodies, antigen binding portions thereof and other binding agents in bacteria, yeast, transgenic animals and chicken eggs are also alternatives for cell-based production systems. The main advantages of transgenic animals are potential high yields from renewable sources.

In some embodiments, the VH regions of the first binding domain that binds to EFGR having the amino acid sequence set forth in SEQ ID NOs: 137, 157, 172, 187, 198, 208, 218, 228, 243, 251, or 259. In some embodiments, the VH regions of the second binding domain that binds to c-MET having the amino acid sequence set forth in SEQ ID NOs: 147, 162, 182, 192, 203, 213, 223, 233, 248, 254, or 264. In some embodiments, the VL regions of the first binding domain that binds to EFGR having the amino acid sequence set forth in SEQ ID NOs:138, 158, 173, 188, 199, 209, 219, 229, 244, 252, or 260. In some embodiments, the VL regions of the second binding domain that binds to c-MET having the amino acid sequence set forth in SEQ ID NO:148, 163, 183, 193, 204, 214, 224, 234, 249, 255, or 265.

In some embodiments, the VH and VL regions of the first binding domain that binds to EGFR having the amino acid sequences set forth in SEQ ID NO: 137 and SEQ ID NO: 138, respectively. In some embodiments, the nucleic acid encodes VH and VL polypeptides having the amino acid sequences set forth in SEQ ID NO: 157 and SEQ ID NO: 158, respectively. In some embodiments, the nucleic acid encodes VH and VL polypeptides having the amino acid sequences set forth in SEQ ID NO: 172 and SEQ ID NO: 173, respectively. In some embodiments, the nucleic acid encodes VH and VL polypeptides having the amino acid sequences set forth in SEQ ID NO: 187 and SEQ ID NO: 188, respectively. In some embodiments, the nucleic acid encodes VH and VL polypeptides having the amino acid sequences set forth in SEQ ID NO: 198 and SEQ ID NO: 199, respectively. In some embodiments, the nucleic acid encodes VH and VL polypeptides having the amino acid sequences set forth in SEQ ID NO: 208 and SEQ ID NO: 209, respectively. In some embodiments, the nucleic acid encodes VH and VL polypeptides having the amino acid sequences set forth in SEQ ID NO: 218 and SEQ ID NO: 219, respectively. In some embodiments, the nucleic acid encodes VH and VL polypeptides having the amino acid sequences set forth in SEQ ID NO: 228 and SEQ ID NO: 229, respectively. In some embodiments, the nucleic acid encodes VH and VL polypeptides having the amino acid sequences set forth in SEQ ID NO: 243 and SEQ ID NO: 244, respectively. In some embodiments, the nucleic acid encodes VH and VL polypeptides having the amino acid sequences set forth in SEQ ID NO: 251 and SEQ ID NO: 252, respectively. In some embodiments, the nucleic acid encodes VH and VL polypeptides having the amino acid sequences set forth in SEQ ID NO: 259 and SEQ ID NO: 260, respectively.

In some embodiments, the VH and VL regions of the second binding domain that binds to c-MET having the amino acid sequences set forth in SEQ ID NO: 147 and SEQ ID NO: 148, respectively. In some embodiments, the nucleic acid encodes VH and VL polypeptides having the amino acid sequences set forth in SEQ ID NO: 162 and SEQ ID NO: 163, respectively. In some embodiments, the nucleic acid encodes VH and VL polypeptides having the amino acid sequences set forth in SEQ ID NO: 182 and SEQ ID NO: 183, respectively. In some embodiments, the nucleic acid encodes VH and VL polypeptides having the amino acid sequences set forth in SEQ ID NO: 192 and SEQ ID NO: 193, respectively. In some embodiments, the nucleic acid encodes VH and VL polypeptides having the amino acid sequences set forth in SEQ ID NO: 203 and SEQ ID NO: 204, respectively. In some embodiments, the nucleic acid encodes VH and VL polypeptides having the amino acid sequences set forth in SEQ ID NO: 213 and SEQ ID NO: 214, respectively. In some embodiments, the nucleic acid encodes VH and VL polypeptides having the amino acid sequences set forth in SEQ ID NO: 223 and SEQ ID NO: 224, respectively. In some embodiments, the nucleic acid encodes VH and VL polypeptides having the amino acid sequences set forth in SEQ ID NO: 233 and SEQ ID NO: 234, respectively. In some embodiments, the nucleic acid encodes VH and VL polypeptides having the amino acid sequences set forth in SEQ ID NO: 248 and SEQ ID NO: 249, respectively. In some embodiments, the nucleic acid encodes VH and VL polypeptides having the amino acid sequences set forth in SEQ ID NO: 254 and SEQ ID NO: 255, respectively. In some embodiments, the nucleic acid encodes VH and VL polypeptides having the amino acid sequences set forth in SEQ ID NO: 264 and SEQ ID NO: 265, respectively.

As used herein, the term “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

Nucleic acid molecules encoding the amino acid sequence of the EGFR and c-MET bispecific antibodies, antigen binding portion thereof as well as other binding agents can be prepared by a variety of methods known in the art. These methods include, but are not limited to, preparation of synthetic nucleotide sequences encoding of the EGFR and c-MET bispecific antibodies, antigen binding portion or other binding agent(s). In addition, oligonucleotide-mediated (or site-directed) mutagenesis, PCR-mediated mutagenesis, and cassette mutagenesis can be used to prepare nucleotide sequences encoding the EGFR and c-MET bispecific antibodies or antigen binding portion as well as other binding agents. A nucleic acid sequence encoding at least the EGFR and c-MET bispecific antibodies, antigen binding portion thereof, binding agent, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases or other techniques known in the art. Techniques for such manipulations are disclosed, e.g., by Maniatis et al., Molecular Cloning, Lab. Manual (Cold Spring Harbor Lab. Press, NY, 1982 and 1989), and Ausubel et al., Current Protocols in Molecular Biology (John Wiley & Sons), 1987-1993, and can be used to construct nucleic acid sequences and vectors that encode the EGFR and c-MET bispecific antibodies or antigen binding portion thereof or a VH or VL polypeptide thereof or other binding agent.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., the EGFR and c-MET bispecific antibodies or antigen binding portion thereof or other binding agent) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of the EGFR and c-MET bispecific antibodies or antigen-binding portion thereof as described herein can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of the EGFR and c-MET bispecific antibodies or antigen binding portion thereof or other binding agent as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant EGFR and c-MET antibodies or antigen-binding portions thereof or other binding agents. Known glycolytic genes can also provide very efficient transcriptional control signals. For example, the promoter and terminator signals of the phosphoglycerate kinase gene can be utilized.

Production of the EGFR and c-MET bispecific antibodies or antigen-binding portions thereof or other binding agents in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence(s) (encoding the EGFR and c-MET bispecific antibodies or antigen binding portion thereof or other binding agent or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding the EGFR and c-MET bispecific antibodies or antigen-binding portions thereof or other binding agents include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the EGFR and c-MET variable region(s) or antigen binding portions thereof or other binding agents are then transfected singly with nucleotides encoding the EGFR and c-MET bispecific antibodies or an antibody polypeptide or antigen-binding portion thereof or other binding agent, or are co-transfected with a polynucleotide(s) encoding VH and VL chain coding regions or other binding agents. The transfected recipient cells are cultured under conditions that permit expression of the incorporated coding regions and the expressed antibody chains or intact antibodies or antigen binding portions or other binding agents are recovered from the culture.

In some embodiments, the nucleic acids containing the coding regions encoding the EGFR and c-MET bispecific antibodies or antigen-binding portion thereof or other binding agent are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing EGFR and c-MET antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused nucleotide sequences encoding VH and VL chains can be assembled on the same expression vector.

For transfection of the expression vectors and production of the EGFR and c-MET bispecific antibodies or antigen binding portions thereof or other binding agents, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding the EGFR and c-MET bispecific antibodies or antigen-binding portion thereof or other binding agent can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment. Johnston et al., 240 Science 1538 (1988), as known to one of ordinary skill in the art.

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled the EGFR and c-MET bispecific antibodies and antigen binding portions thereof and other binding agents. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1a promoter, such as that from Chinese hamster cells. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof or other binding agents as described herein. E. coli K12 strains such as E. coli W3110, Bacillus species, enterobacteria such as Salmonella typhimurium or Serratia marcescens, and various Pseudomonas species can be used. Plasmid vectors containing replicon and control sequences that are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of the EGFR and c-MET bispecific antibodies and antigen binding portions thereof and other binding agents in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof or other binding agents.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells; 293 cells, including 293-6E cells; CHO cells, including CHO-S and DG44 cells; PERC6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more the EGFR and c-MET bispecific antibodies or antigen-binding portions thereof or other binding agents can be produced in vivo in an animal that has been engineered or transfected with one or more nucleic acid molecules encoding the polypeptides, according to any suitable method.

In some embodiments, an antibody or antigen-binding portion thereof is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695-713 (2003).

Many vector systems are available for the expression of the VH and VL chains in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H2L2 antibodies or antigen-binding portions thereof. The co-expression can occur by using either the same or different plasmids in the same host. Nucleic acids encoding the VH and VL chains or antigen binding portions thereof can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies, antigen-binding portions thereof via either route could be transfected with plasmids encoding additional copies of peptides, VH, VL, or VH plus VL chains in conjunction with additional selectable markers to generate cell lines with enhanced properties, such as higher production of assembled the EGFR and c-MET bispecific antibodies or antigen binding portions thereof or other binding agents or enhanced stability of the transfected cell lines.

Additionally, plants have emerged as a convenient, safe and economical alternative expression system for recombinant antibody production, which are based on large scale culture of microbes or animal cells. EGFR and c-MET bispecific binding antibodies or antigen binding portions thereof or other binding agents can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; and WO 0129242. Several plant-derived antibodies have reached advanced stages of development, including clinical trials (see, e.g., Biolex, N.C.).

For intact antibodies, the variable regions (VH and VL regions) of the the EGFR and c-MET bispecific antibodies are typically linked to at least a portion of an immunoglobulin constant region (Fc) or domain, typically that of a human immunoglobulin. Human constant region DNA sequences can be isolated in accordance with well-known procedures from a variety of human cells, such as immortalized B-cells (WO 87/02671). An EGFR and c-MET bispecific binding antibody can contain both light chain and heavy chain constant regions. The heavy chain constant region can include CH1, hinge, CH2, CH3, and, optionally, CH4 regions. In some embodiments, the CH2 domain can be deleted or omitted.

Techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies that specifically bind to EGFR and c-MET. Single chain antibodies are formed by linking the heavy and light chain variable regions of the Fv region via an amino acid bridge, resulting in a single chain polypeptide. Techniques for the assembly of functional Fv portions in E. coli can also be used (see, e.g. Skerra et al., Science 242:1038-1041 (1988); which is incorporated by reference herein in its entirety).

In some embodiments, an antigen binding portion or other binding agent comprises one or more scFvs. An scFv can be, for example, a fusion protein of the variable regions of the heavy (VH) and light chain (VL) variable regions of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96. Methods for making scFv molecules and designing suitable peptide linkers are described in, for example, U.S. Pat. Nos. 4,704,692; 4,946,778; Raag and Whitlow, FASEB 9:73-80 (1995) and Bird and Walker, TIBTECH, 9: 132-137 (1991). scFv-Fcs have been described by Sokolowska-Wedzina et al., Mol. Cancer Res. 15(8):1040-1050, 2017.

In some embodiments, an antigen binding portion or other binding agent is a single-domain antibody is an antibody portion consisting of a single monomeric variable antibody domain. Single domains antibodies can be derived from the variable domain of the antibody heavy chain from camelids (e.g., nanobodies or VHH portions). Furthermore, a single-domain antibody can be an autonomous human heavy chain variable domain (aVH) or VNAR portions derived from sharks (see, e.g., Hasler et al., Mol. Immunol. 75:28-37, 2016).

Techniques for producing single domain antibodies (DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize the target antigen (see, e.g., Maass et al., 2007). PCR primers that amplify alpaca VHH coding sequences have been identified and can be used to construct alpaca VHH phage display libraries, which can be used for antibody fragment isolation by standard biopanning techniques well known in the art (see, e.g., Maass et al., 2007).

Techniques for making multispecific antibodies include, but are not limited to, recombinant co-expression of two immunoglobulin heavy chain-light chain pairs having different specificities (see, e.g., Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; Carter (2001), J Immunol Methods 248, 7-15). Multi-specific antibodies may also be made by engineering electrostatic steering effects for making antibody Fc-heterodimeric molecules (see, e.g., WO 2009/089004A1); cross-linking of two or more antibodies or antigen binding portions thereof (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody portions (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” also can be binding agents (see, e.g. US 2006/0025576A1).

The binding agents (e.g., antibodies or antigen binding portions) herein also include a “Dual Acting FAb” or “DAF” comprising an antigen binding site that binds to two different antigens (see, e.g., US 2008/0069820 and Bostrom et al., 2009, Science 323:1610-14). “Crossmab” antibodies are also included herein (see e.g. WO 2009/080251, WO 2009/080252, WO2009/080253, WO2009/080254, and WO2013/026833).

In some embodiments, the binding agents comprise different antigen-binding sites, fused to one or the other of the two subunits of the Fc domain; thus, the two subunits of the Fc domain may be comprised in two non-identical polypeptide chains. Recombinant co-expression of these polypeptides and subsequent dimerization leads to several possible combinations of the two polypeptides. To improve the yield and purity of the bispecific molecules in recombinant production, it will thus be advantageous to introduce in the Fc domain of the binding agent a modification promoting the association of the desired polypeptides.

Generally, this method involves replacement of one or more amino acid residues at the interface of the two Fc domains by charged amino acid residues so that homodimer formation becomes electrostatically unfavorable but heterodimerization electrostatically favorable.

In some embodiments, a binding agent is a “bispecific T cell engager” or BiTE (see, e.g., WO2004/106381, WO2005/061547, WO2007/042261, and WO2008/119567). This approach utilizes two antibody variable domains arranged on a single polypeptide. For example, a single polypeptide chain can include two single chain Fv (scFv) portions, each having a variable heavy chain (VH) and a variable light chain (VL) domain separated by a polypeptide linker of a length sufficient to allow intramolecular association between the two domains. This single polypeptide further includes a polypeptide spacer sequence between the two scFvs. Each scFv recognizes a different epitope, and these epitopes may be specific for different proteins, such that both proteins are bound by the BiTE.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In some embodiments, a binding agent that is a bispecific antibody is composed of a single polypeptide chain comprising two single chain FV portions (scFV) fused to each other by a peptide linker.

In some embodiments, a binding agent is multispecific, such as an IgG-scFV. IgG-scFv formats include IgG(H)-scFv, scFv-(H)IgG, IgG(L)-scFv, svFc-(L)IgG, 2scFV-IgG and IgG-2scFv. These and other bispecific antibody formats and methods of making them have been described in for example, Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

IgG-like dual-variable domain antibodies (DVD-Ig) have been described by Wu et al., 2007, Nat Biotechnol 25:1290-97; Hasler et al., Mol. Immunol. 75:28-37, 2016 and in WO 08/024188 and WO 07/024715. Triomabs have been described by Chelius et al., MAbs 2(3):309-319, 2010. 2-in-1-IgGs have been described by Kontermann et al., Drug Discovery Today 20(7):838-847, 2015. Tanden antibody or TandAb have been described by Kontermann et al., id. ScFv-HSA-scFv antibodies have also been described by Kontermann et al. (id.).

Intact (e.g., whole) antibodies, their dimers, individual light and heavy chains, or antigen binding portions thereof and other binding agents can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure EGFR and c-MET binding antibodies or antigen binding portions thereof or other binding agents of at least about 90% to 95% homogeneity are advantageous, as are those with 98% to 99% or more homogeneity, particularly for pharmaceutical uses. Once purified, partially or to homogeneity as desired, an intact EGFR and c-MET bispecific antibodies or antigen binding portions thereof or other binding agent can then be used therapeutically or in developing and performing assay procedures, immunofluorescent staining, and the like. See generally, Vols. I & II Immunol. Meth. (Lefkovits & Pernis, eds., Acad. Press, NY, 1979 and 1981).

### Antibody Drug Conjugates

In some embodiments, an EGFR and c-MET bispecific antibodies, antigen binding portion or other binding agent as described herein is part of the EGFR and c-MET bispecific antibodies drug conjugate (also referred to as an EGFR and c-MET bispecific conjugate or EGFR and c-MET bispecific ADC). In some embodiments, the the EGFR and c-MET bispecific antibodies, antigen binding portion or other binding agent is attached to at least one linker, and at least one drug is attached to each linker. As used herein, in the context of a conjugate, the term “drug” refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

### Cytotoxic Agents

In some embodiments, an EGFR and c-MET bispecific conjugate includes at least one drug (or termed as “drug unit”) that is cytotoxic agent. A “cytotoxic agent” refers to an agent that has a cytotoxic effect on a cell. A “cytotoxic effect” refers to the depletion, elimination and/or the killing of a target cell(s). Cytotoxic agents include, for example, tubulin disrupting agents, topoisomerase inhibitors, DNA minor groove binders, and DNA alkylating agents.

Tubulin disrupting agents include, for example, auristatins, dolastatins, tubulysins, colchicines, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine), MMAF (N-methylvaline-valine-dolaisoleuine-dolaproine-phenylalanine) and AFP (see WO2004/010957 and WO2007/008603). Other auristatin like compounds are disclosed in, for example, Published US Application Nos. US2021/0008099, US2017/0121282, US2013/0309192 and US2013/0157960. Dolastatins include, for example, dolastatin 10 and dolastatin 15 (see, e.g., Pettit et al., J. Am. Chem. Soc., 1987, 109, 6883-6885; Pettit et al., Anti-Cancer Drug Des., 1998, 13, 243-277; and Published US Application US2001/0018422). Additional dolastatin derivatives contemplated for use herein are disclosed in U.S. Pat. No. 9,345,785, incorporated herein by reference. In some embodiments, the tubulin disrupting agent is MMAE.

Tubulysins include, but are not limited to, tubulysin D, tubulysin M, tubuphenylalanine and tubutyrosine. WO2017/096311 and WO/2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52.

Maytansinoids include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl) +/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Maytansinoid drug moieties also include those having modifications such as: C-9-SH (U.S. Pat. No. 4,424,219) (prepared by the reaction of maytansinol with H2S or P2S5); C-14-alkoxymethyl(demethoxy/CH2OR) (U.S. Pat. No. 4,331,598); C-14-hydroxymethyl or acyloxymethyl (CH2OH or CH2OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudiflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Hemiasterlins include but are not limited to, hemiasterlin and HTI-286.

Other tubulin disrupting agents include taccalonolide A, taccalonolide B, taccalonolide AF, taccalonolide AJ, taccalonolide AI-epoxide, discodermolide, epothilone A, epothilone B, and laulimalide.

In some embodiments, a cytotoxic agent can be a topoisomerase inhibitor, such as a camptothecin. Exemplary camptothecins include, for example, camptothecin, irinotecan (also referred to as CPT-11), belotecan, (7-(2-(N-isopropylamino)ethyl)camptothecin), topotecan, 10-hydroxy-CPT, SN-38, exatecan and the exatecan analog DXd (see US20150297748). Other camptothecins are disclosed in WO1996/021666, WO00/08033, US2016/0229862 and WO2020/156189.

In some embodiments, a cytotoxic agent is a duocarmcycin, including the synthetic analogues, KW-2189 and CBI-TMI.

### Immune Modulatory Agents

In some embodiments, a drug is an immune modulatory agent. An immune modulatory agent can be, for example, a TLR7 and/or TLR8 agonist, a STING agonist, a RIG-1 agonist or other immune modulatory agent.

In some embodiments, a drug is an immune modulatory agent, such as a TLR7 and/or TLR8 agonist. In some embodiments, a TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences).

In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, and a tetrahydropyridopyrimidine. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463.

In some embodiments, a TLR8 agonist can be any of the compounds described WO2018/170179, WO2020/056198 and WO2020056194.

Other TLR7 and TLR8 agonists are disclosed in, for example, WO2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6,043,238, US20180086755 (Gilead), WO2017216054 (Roche), WO2017190669 (Shanghai De Novo Pharmatech), WO2017202704 (Roche), WO2017202703 (Roche), WO20170071944 (Gilead), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics), WO2018198091 (Novartis AG), and US20170131421 (Novartis AG).

In some embodiments, an immune modulatory agent is a STING agonist. Examples of STING agonists include, for example, those disclosed in WO2020059895, WO2015077354, WO2020227159, WO2020075790, WO2018200812, and WO2020074004.

In some embodiments, an immune modulatory agent is a RIG-1 agonist. Examples of RIG-1 agonists include KIN1148, SB-9200, KIN700, KIN600, KIN500, KIN100, KIN101, KIN400 and KIN2000.

### Toxins

In some embodiments, a drug is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

### Radioisotopes

In some embodiments, a drug is a radioactive atom. A variety of radioactive isotopes are available for the production of radioconjugates. Examples include I131, I125, Y90, Re186, Re188, Sm153, Bi213, P32, Pb212 and radioactive isotopes of Lutetium (e.g., Lu177).

### PROTACs

In some embodiments, a drug is a proteolysis targeted chimera (PROTAC). PROTACs are described in, for example, Published US Application Nos. 20210015942, 20210015929, 20200392131, 20200216507, US20200199247 and US20190175612; the disclosures of which are incorporated by reference herein.

### Linkers

The EGFR and c-MET bispecific conjugates typically comprise at least one linker, each linker having at least one drug attached to it. Typically, a conjugate includes a linker between the EGFR and c-MET bispecific antibodies (or antigen binding portion thereof or other binding agent) and the drug (in some cases termed “drug unit”). In various embodiments, a linker may be a protease cleavable linker, an acid-cleavable linker, a disulfide linker, a disulfide-containing linker, or a disulfide-containing linker having a dimethyl group adjacent the disulfide bond (e.g., an SPDB linker) (see, e.g., Jain et al., Pharm. Res. 32:3526-3540 (2015); Chari et al., Cancer Res. 52:127-131 (1992); U.S. Pat. No. 5,208,020), a self-stabilizing linker (see, e.g., WO2018/031690 and WO2015/095755 and Jain et al., Pharm. Res. 32:3526-3540 (2015)), a non-cleavable linker (see, e.g., WO2007/008603), a photolabile linker, and/or a hydrophilic linker (see, e.g., WO2015/123679).

In some embodiments, a linker is a cleavable linker that is cleavable under intracellular conditions, such that cleavage of the linker releases the drug from the antibody (or antigen binding portion thereof or other binding agent) and/or linker in the intracellular environment. For example, in some embodiments, a linker is cleavable by a cleaving agent that is present in the intracellular environment (e.g., within a lysosome or endosome or caveolae). A linker can be, for example, a peptidyl linker that is cleaved by an intracellular peptidase or protease enzyme, including, but not limited to, a lysosomal or endosomal protease (see, e.g., WO2004/010957, US20150297748, US2008/0166363, US20120328564 and US20200347075). Typically, a peptidyl linker is at least one amino acid long or at least two amino acids long. Intracellular cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in target antigen-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). Other such linkers are described, for example, in U.S. Pat. No. 6,214,345. In specific embodiments, the peptidyl linker cleavable by an intracellular protease is a Val-Cit linker or a Phe-Lys linker (see, e.g., U.S. Pat. No. 6,214,345, which describes the synthesis of doxorubicin with the val-cit linker) or Gly-Gly-Phe-Gly (SEQ ID NO: 60) linker (see, e.g., US2015/0297748). One advantage of using intracellular proteolytic release of the drug is that the drug is typically attenuated when conjugated and the serum stabilities of the conjugates are typically high. See also U.S. Pat. No. 9,345,785.

As used herein, the terms “intracellularly cleaved” and “intracellular cleavage” refer to a metabolic process or reaction inside a cell on an antibody drug conjugate, whereby the covalent attachment, e.g., the linker, between a drug (e.g., a cytotoxic agent) and the antibody is broken, resulting in the free drug, or other metabolite of the conjugate dissociated from the antibody inside the cell. The cleaved moieties of the conjugate are thus intracellular metabolites.

In some embodiments, a cleavable linker is pH-sensitive, i.e., sensitive to hydrolysis at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; and 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, a hydrolyzable linker is a thioether linker (such as, for example, a thioether attached to the drug via an acylhydrazone bond (see, e.g., U.S. Pat. No. 5,622,929)).

In some embodiments, a linker is cleavable under reducing conditions (e.g., a disulfide linker). A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (see, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In some embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12). In some embodiments, the linker is not cleavable, such as a maleimidocaproyl linker, and the drug is released by antibody degradation. (See U.S. Publication No. 2005/0238649).

In some embodiments, a linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of the antibody drug conjugate (ADC), are cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or drug (the “control sample”) for a predetermined time period (e.g., 2, 4, 8, 16, or 24 hours) and then comparing the amount of unconjugated antibody or drug present in the ADC sample with that present in control sample, as measured, for example, by high performance liquid chromatography.

In some embodiments, a linker promotes cellular internalization. In some embodiments, a linker promotes cellular internalization when conjugated to the drug such as a cytotoxic agent (i.e., in the milieu of the linker-drug moiety of the ADC as described herein). In yet other embodiments, a linker promotes cellular internalization when conjugated to both the drug and the EGFR and c-MET bispecific antibodies (i.e., in the milieu of the ADC as described herein).

A variety of linkers that can be used with the present compositions and methods are described in WO 2004010957. In some embodiments, a protease cleavable linker comprises a thiol-reactive spacer and a dipeptide. In some embodiments, the protease cleavable linker consists of a thiol-reactive maleimidocaproyl spacer, a valine-citrulline dipeptide, and a p-amino-benzyloxycarbonyl spacer.

In some embodiments, an acid cleavable linker is a hydrazine linker or a quaternary ammonium linker (see WO2017/096311 and WO2016/040684.)

In some embodiments, a linker is a self-stabilizing linker comprising a maleimide group as described in U.S. Pat. No. 9,504,756.

In some embodiments, a linker is a hydrophilic linker, such as, for example, the hydrophilic peptides in WO2015/123679 and the sugar alcohol polymer-based linkers disclosed in WO2013/012961 and WO2019/213046.

In some embodiments, the binging agents herein disclosed may be connected to a linker as disclosed in WO2023280227, the contents of which are incorporated by reference in their entireties.

In other embodiments, conjugates of the EGFR and c-MET bispecific antibodies (or antigen binding portion or other binding agent) and a drug may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Chelating agents for conjugation of a radionucleotide(s) to an antibody, antigen binding portion thereof or other binding agent have been described in, for example WO94/11026.

The conjugates of the EGFR and c-MET bispecific antibodies (or antigen binding portion or other binding agent) include, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A.).

In some embodiments, a linker is attached to a terminus of an amino acid sequence of an antibody, antigen binding portion or other binding agent or can be attached to a side chain modification of an antibody, antigen binding portion or other binding agent, such as the side chain of a lysine, serine, threonine, cysteine, tyrosine, aspartic acid, a non-natural amino acid residue, glutamine, or glutamic acid residue. An attachment between an antibody, antigen binding portion or other binding agent and a linker or drug can be via any of a number of bonds, for example but not limited to, an amide bond, an ester bond, an ether bond, a carbon-nitrogen bond, a carbon-carbon single double or triple bond, a disulfide bond, or a thioether bond. Functional groups that can form such bonds include, for example, amino groups, carboxyl groups, aldehyde groups, azide groups, alkyne and alkene groups, ketones, carbonates, carbonyl functionalities bonded to leaving groups such as cyano and succinimidyl and hydroxyl groups.

In some embodiments, a linker is attached to an antibody, antigen binding portion or other binding agent at an interchain disulfide. In some embodiments, a linker is connected to an antibody, antigen binding portion or other binding agent at a hinge cysteine residue. In some embodiments, a linker is attached to an antibody, antigen binding portion or other binding agent at an engineered cysteine residue. In some embodiments, a linker is connected to an antibody, antigen binding portion or other binding agent at a lysine residue. In some embodiments, a linker is connected to an antibody, antigen binding portion or other binding agent at an engineered glutamine residue. In some embodiments, a linker is connected to an antibody, antigen binding portion or other binding agent at an unnatural amino acid engineered into the heavy chain.

In some embodiments, a linker is attached to an antibody, antigen binding portion or other binding agent via a sulfhydryl group. In some embodiments, a linker is attached to an antibody, antigen binding portion or other binding agent via a primary amine. In some embodiments, a linker is attached via a link created between an unnatural amino acid on an antibody, antigen binding portion or other binding agent by reacting with oxime bond that was formed by modifying a ketone group with an alkoxyamine on a drug.

In some embodiments, a linker is attached to an antibody, antigen binding portion or other binding agent via Sortase A linker. A Sortase A linker can be created by a Sortase A enzyme fusing an LPXTG recognition motif (SEQ ID NO: 61) to an N-terminal GGG motif to regenerate a native amide bond.

In some embodiments, the conjugate of the present disclosure comprises: one of the EGFR and c-MET antibodies, antigen binding portions thereof, and other binding agents, at least one linker attached to the binding agent; at least one drug unit, wherein each drug unit is attached to a linker, and wherein the linker optionally comprises at least one polar group.

In some embodiments, in the conjugate of the present disclosure, the linker is derived from a linker compound, or a stereoisomer or salt thereof, and the linker compound comprises: a linker unit; a stretcher group connected to the linker unit, an optional amino acid unit; and the at least one polar group; wherein: the stretcher group has an attachment site to the binding agent and an attachment site to the amino acid unit (when present) or the linker subunit; the amino acid unit (when present) has an attachment site to the stretcher group and an attachment site to the linker unit; and the linker unit has an attachment site to the amino acid unit (when present) or to the stretcher group and to the at least one drug unit.

Some of the components and variations of the linker (and the linker compound) are exemplified and demonstrated by the “linker embodiments” herein provided.

### Linker Embodiments

The linker (and linker compound) of the present disclosure is further illustrated by the following embodiments which should not be construed as limiting.

Embodiment 1. A linker compound, or a stereoisomer or salt thereof, comprising:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein the polymer unit comprises the formula:

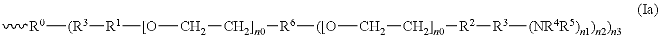

or a stereoisomer or salt thereof, wherein:
R⁰ is a functional group for attachment to a subunit of the amino acid unit;
each R¹ and R² are independently a bond or C₁-C₆ alkylene;
each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH2-CH₂, —O—C(O)—NR^{a}—C₁-C₁₂ alkylene, —O—CH₂—CH(OH)—C(O)—, —O—CH₂—CH(OH)—C(O)—NR^{a}— C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈NR^{a}]₁₋₈, triazolyl, —C₁-C₁₂ alkylene-triazolyl-, —N(polyhydroxyl group)-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R⁶ is selected from:

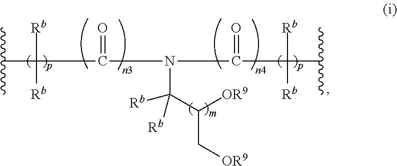

wherein:
   each n3 and n4 are independently 0-1,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4,
   each v is independently 1-6, and
n2 is 1;

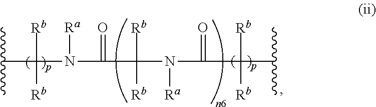

wherein:
   each R^{a} is independently H or C₁₋₆ alkyl,
   each R^{b} is independently H or C₁₋₆ alkyl,
   n6 is 1-10,
   each p is independently 0-6, and
n2 is 1;

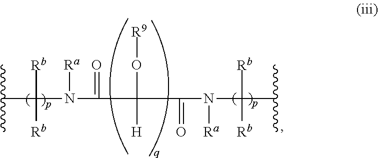

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   q is 1-8,
   each v is independently 1-6, and
n2 is 1;

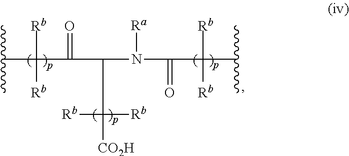

wherein:
   each R^{a} is independently H or C₁₋₆ alkyl,
   each R^{b} is independently H or C₁₋₆ alkyl,
   each p is independently 0-6, and
n2 is 1;

—R¹⁰—[O—CH₂—CH₂]₁₋₈—R¹⁰—, wherein: (v)

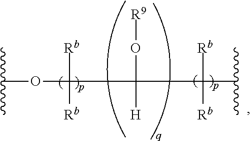

each R¹⁰ is independently R,
each R^{b} is independently H or C₁₋₆ alkyl,
each p is independently 1-6,
each R⁹ is independently H, acetyl, —P(═O)(OH)₂, or —(CH₂)ᵥ—O—S(═O)₂(OH), and
q is 1-8;
n2 is 1; and

—N—(R¹—X—R²—)₂, wherein: (vi)
each X is independently —NR^{a}—C(O)— or —C(O)NR^{a}—, and
n2 is 2; and
the wavy line (˜) indicates an attachment site of the amino acid unit to R⁰;
each n0 is independently 2-26;
each n1 is independently 1-6; and
n3 is 1-6.

Embodiment 2. A linker compound, or a stereoisomer or salt thereof, comprising:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises the formula:

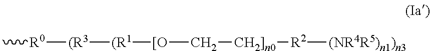

or a stereoisomer or salt thereof, wherein:
R⁰ is a functional group for attachment to a subunit of the amino acid unit;
each R¹ and R² are independently a bond or C₁-C₆ alkylene;
each R³ is independently —N(polyhydroxyl group)-, triazolyl, —C₁-C₁₂ alkylene-triazolyl-,

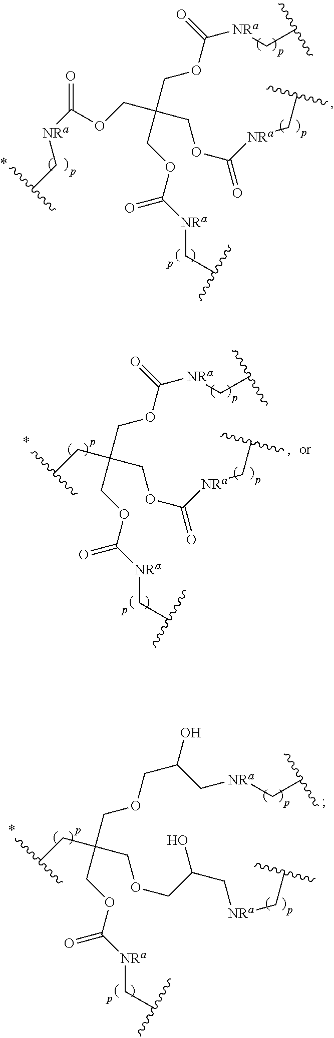

each R⁴ and R⁵ are independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;

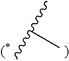

indicates an attachment site of R³ to R⁰
the wavy line

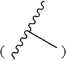

indicates an attachment site of the R³ to R¹;
each p is 1-6;
each n0 is independently 2-8;
each n1 is independently 1-6; and
n3 is 1-6.

Embodiment 3. A linker compound, or a stereoisomer or salt thereof, comprising:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises the formula:

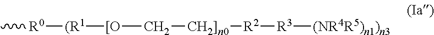

or a stereoisomer or salt thereof, wherein:
(i) R⁰ is a functional group for attachment to a subunit of the amino acid unit;
each R¹ and R² are independently a bond or C₁-C₆ alkylene;
R³ is —C(O)—;
R⁴ is H;
R⁵ is independently a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate; the wavy line (˜) indicates the attachment site of the amino acid unit to R⁰;
n0 is independently 2-26;
n1 is 1-6; and
n3 is 1-6;
(ii) R⁰ is —C(O)—;
R¹, R², and R³ are each a bond;
R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the amino acid unit to R⁰;
n0 is 6;
n1 is 1-6; and
n3 is 1;
(iii) R⁰ is a functional group for attachment to a subunit of the amino acid unit;
R¹ and R² are each, independently, a bond or C₁-C₆ alkylene;
R³ is —NRₐ—C(O)—C₁-C₁₂ alkylene-C(O)—, wherein the alkylene is substituted with —SO₃H;
R^{a} is H or C₁₋₆ alkyl;
R⁴ and R⁵ are each independently H, a carboxyl-containing moiety, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the amino acid unit to R⁰;
each n⁰ is independently 1-26;
n¹ is 1-6; and
n³ is 1-6; or
(iv) R⁰ is

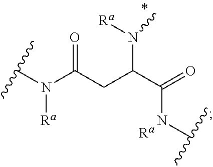

each R¹ is independently a bond or C₁-C₆ alkylene;
R² and R³ are each a bond;
R⁴ and R⁵ are each independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;
the wavy line

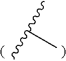

indicates an attachment site of R⁰ to the remainder of the polymer unit;
the wavy line (˜*) indicates an attachment site of the amino acid unit to R⁰;
n0 is 1-8;
n1 is 1-6; and
n3 is 2.

Embodiment 4. A linker compound, or a stereoisomer or salt thereof, comprising:
(a) a linker unit having from 1 to 4 attachment sites for a drug unit, said linker unit comprising a moiety of formula:

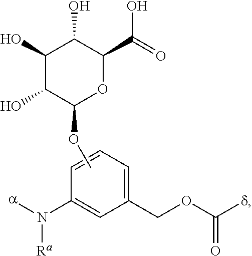

or a stereoisomer or salt thereof, wherein:
α—represents a direct or indirect attachment site to an amino acid unit;
δ—represents an attachment site to the drug unit or for a linking group attached to the the drug unit; and
R^{a} is H or C₁₋₆ alkyl;
(b) the amino acid unit having from 1 to 12 amino acid subunits; and
(c) at least one polar group attached to the amino acid unit, wherein the polar group comprises a polymer unit, optionally a sugar unit, and optionally a carboxyl unit.

Embodiment 5. A linker compound, or a stereoisomer or salt thereof, comprising:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises:
(i) an optionally substituted polyamide comprising a formula

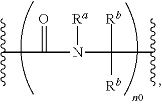

or a stereoisomer thereof, wherein each R^{a} is independently H or C₁₋₆ alkyl and each R^{b} is independently H or C₁₋₆ alkyl, and n⁰ is independently 2-26;
(ii) a substituted polyether comprising the formula

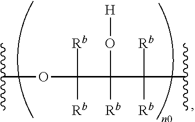

or a stereoisomer thereof, wherein each R^{b} is independently H or C₁₋₆ alkyl, and n0 is independently 2-26; or
(iii) combinations thereof.

Embodiment 6. A linker compound, or a stereoisomer or salt thereof, comprising:
(a) a linker unit having from 1 to 4 attachment sites for a drug unit and having one of following structures (i) or (ii):

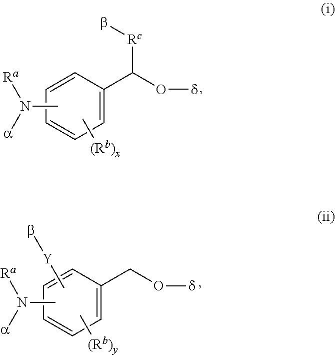

(b) at least one polar group comprising a polymer unit, optionally a sugar unit, optionally a carboxyl unit, and combinations thereof; and
(c) optionally a stretcher group having an attachment site for a EGFR and c-MET binding agent;
wherein:
α—is an attachment site to an the amino acid unit, the amino acid unit being enzyme-cleavable group;
β—is an attachment site to the at least one polar group;
δ—is H, an attachment site to the drug units, or an attachment site to a linking group attached to the drug unit;
the polymer unit comprises a polyamide, a polyether, or a combination thereof, wherein the polyether comprises a hydroxyl group, a polyhydroxyl group, a sugar group, a carboxyl group, or combinations thereof;
each R^{a} independently is H or C₁-C₆ alkyl;
each R^{b} independently is halo, C₁₋₆ alkyl, an attachment site to the drug units, or an attachment site to the at least one polar group;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl, a group containing triazolyl, or combinations thereof.

Embodiment 7. The linker compound of any one of Embodiments 1-6, wherein the polar group comprises at least one sugar unit having the following formula:

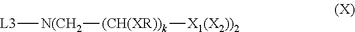

or a stereoisomer or salt thereof, wherein:
each X is independently selected from NH and O;
each R is independently selected from hydrogen, acetyl, a monosaccharide, a disaccharide, and a polysaccharide;
each X₁ is independently selected from CH₂ and C(O);
each X₂ is independently selected from H, OH and OR;
k is 1 to 10; and
L3 is a point of attachment to a remainder of the polar group.

Embodiment 8. The linker compound of Embodiment 7, wherein the at least one sugar unit has one of the following structures (XII) or (XIII):

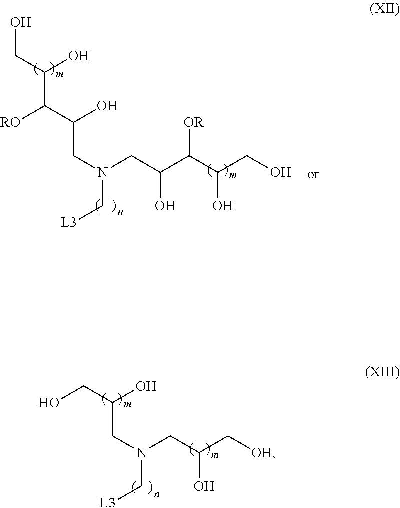

or a stereoisomer or salt thereof, wherein:
each R is independently selected from hydrogen, a monosaccharide, a disaccharide and a polysaccharide;
m is 1 to 8; and
n is 0 to 4.

Embodiment 9. The linker compound of Embodiments 7 or 8, the polar group has a formula selected from:

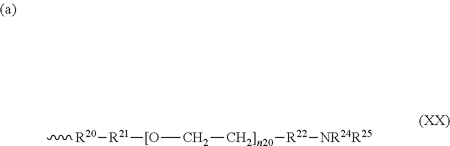

or a stereoisomer a salt thereof, wherein:
R²⁰ is an attachment group to site β or to site R^{b}, or to the amino acid unit, the amino acid unit being an enzyme-cleavable group;
R²¹ and R²² are each, independently, a bond or C₁-C₃ alkylene;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is the sugar unit of formula (XII) or (XIII); or —NR²⁴R²⁵ together from a C₃-C₈ heterocycle; and
n20 is 2 to 26; or

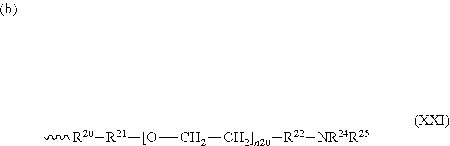

or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene; one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is the sugar unit of formula (XII) or (XIII); and the other of R²⁴ and R²⁵ is a polyethylene glycol, optionally having 1 to 24 ethylene glycol subunits; and
n20 is 2 to 26; or

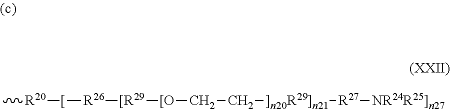

or a stereoisomer or salt thereof, wherein:
R²⁰ is an attachment group to site β or to site R^{b}, or to the enzyme-cleavable group;
R²⁶ and R²⁷ are each optional and are, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—; one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is the sugar unit of formula (XII) or (XIII); and the other of R²⁴ and R²⁵ is selected from H; polyhydroxyl group; substituted polyhydroxyl group; —C(O)— polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, wherein R²⁸ is the sugar unit of formula (XII) or (XIII); and polyethylene glycol, optionally having 1 to 24 ethylene glycol subunits; or —NR²⁴R²⁵ together from a C₃-C₈ heterocycle;
each R²⁹ is optional and independently selected from —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
n20 is 2 to 26;
n21 is 1 to 4; and
n27 is 1 to 4, or

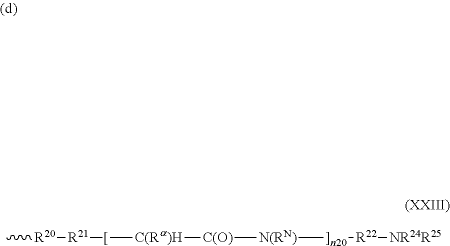

or a stereoisomer or salt thereof, wherein:
R²⁰ is an attachment group to site β or to site R^{b}, or to the enzyme-cleavable group;
R²¹ is a bond, C₁-C₃ alkylene,
   —C₁-C₃alkylene-[O—CH₂—CH₂—]ₙ₂₀, —[CH₂—CH₂—O]ₙ₂₀—C₁-C₃alkylene- or —C₁-C₃alkylene-[O—CH₂—CH₂—]ₙ₂₀—C(O)—;
R²² is C₁-C₃ alkylene,
   —C₁-C₃alkylene-[O—CH₂—CH₂—]ₙ₂₀, —[CH₂—CH₂—O]ₙ₂₀—C₁-C₃alkylene- or —C₁-C₃alkylene-[O—CH₂—CH₂—]ₙ₂₀—C(O)—;
each R^{a} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, wherein R²⁸ is the sugar unit of formula (XII) or (XIII); or —NR²⁴R²⁵ together from a C₃-C₈ heterocycle; and
each n20 is independently 2 to 26, or

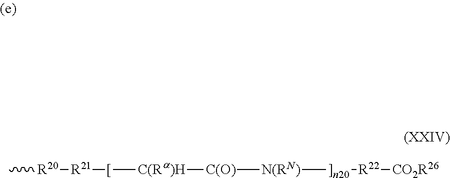

or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, or
—C₁-C₃alkylene[O—CH₂—CH₂—]ₙ₂₀;
each R^{a} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, wherein R²⁸ is the sugar unit of formula (XII) or (XIII); or —NR²⁴R²⁵ together from a C₃-C₈ heterocycle;
R²⁶ is H or C₁-C₄ alkyl; and
each n20 is independently 2 to 26,
with the proviso that at least one R⁰ or R^{N} is —R²²—NR²⁴R²⁵; or

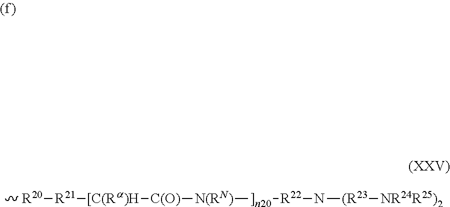

or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, or
—C₁-C₃alkylene-[O—CH₂—CH₂—]ₙ₂₀;
each R⁰ is independently H or —R²²—NR²⁴R²⁵;
each R^{N} is independently H or C₁-C₆ alkyl;
each R²³ is independently C₁-C₆ alkylene;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, wherein R²⁸ is the sugar unit of formula (XII) or (XIII); or —NR²⁴R²⁵ together from a C₃-C₈ heterocycle; and each n20 is independently 2 to 26.

Embodiment 10. The linker compound of Embodiment 7 or 8, the polar group has a formula selected from the following:

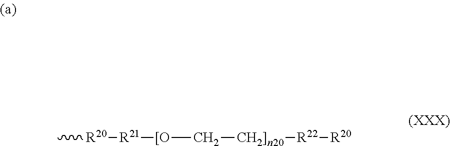

or a stereoisomer or salt thereof, wherein:
R²⁰ is an attachment group to site β or to site R^{b}, or to the amino acid unit, the amino acid unit being an enzyme-cleavable group;
R²¹ and R²² are each independently, a bond or C₁-C₃ alkylene groups;
R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)-alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

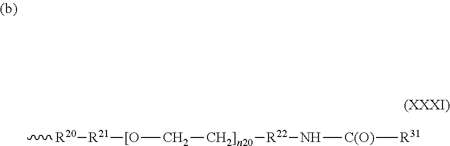

or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

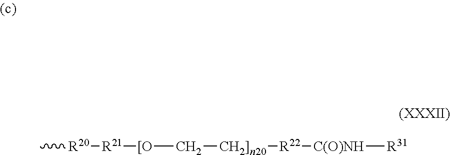

or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch, independently, having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and
n20 is 2 to 26;

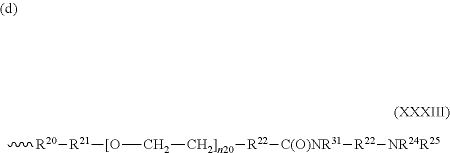

or a stereoisomer or salt thereof, wherein:
R²⁰ is an attachment group to site β or to site R^{b}, or to the enzyme-cleavable group;
R³¹ is H or R²²—NR²⁴R²⁵;
R²¹ and R²² are each, independently, a bond or C₁-C₃ alkylene groups;
R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H; and
n20 is 2 to 26;

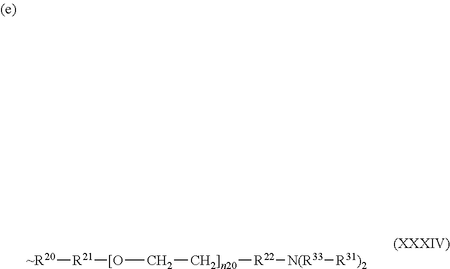

or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, C₁-C₃ alkylene-C(O), —C(O)—C₁-C₃ alkylene, or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

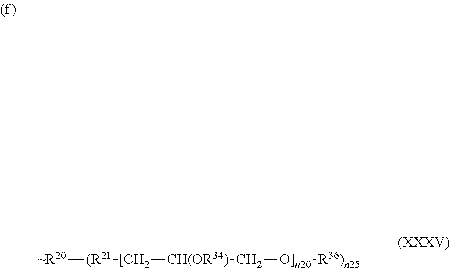

or a stereoisomer or salt thereof, wherein:
R²⁰ is an attachment group to site β or to site R_{b}, or to the enzyme-cleavable group;
each R²¹ is independently a bond, —O— or C₁-C₃ alkylene group;
each R³⁴ is independently H, —[CH₂—CH(OH)—CH₂—O]ₙ₂₀—R³⁶, —C(O)—NR²⁴R²⁵ or C(O)N(R^{N})—C₁-C₆alkylene-NR²⁴R²⁵;
R^{N} is H or C₁-C₄alkyl;
R²⁴ and R²⁵ are each independently selected from a H; polyhydroxyl group; or substituted polyhydroxyl group, provided that both R²⁴ and R²⁵ are not H;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)—NR²⁴R²⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; substituted —C(O)-polyhydroxyl group; provided that both R⁴⁴ and R⁴⁵ are not H;
each n20 is independently 1 to 26; and
n25 is 1 or 2

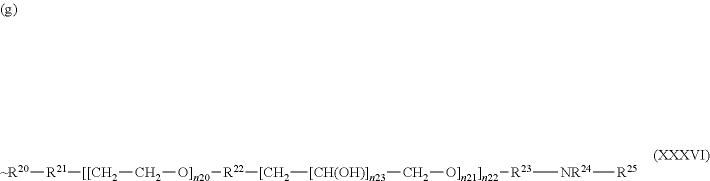

or a stereoisomer or salt thereof, wherein:
R²⁰ is an attachment group to site β or to site R^{b}, or to the enzyme-cleavable group;
R²¹, R²² and R²³ are each independently a bond or C₁-C₃ alkylene group;
R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H;
each n20 is independently 0 to 26, and each n21 is independently 0 to 26, with the proviso that at least one of n20 or n21 is 2 to 26;
n22 is 1 to 5;
each n23 is independently 1 or 2;

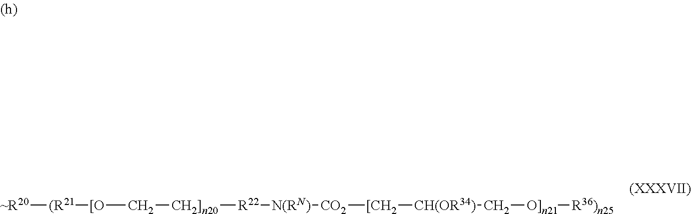

or a stereoisomer or salt thereof, wherein:
R²⁰ is an attachment group to site β or to site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; or substituted polyhydroxyl group, provided that both R²⁴ and R²⁵ are not H;
each R³⁴ is independently H, —[CH₂—CH(OH)—CH₂—O]ₙ₂₀—R³⁶ or —C(O)N(R^{N})—C₁-C₆alkylene-NR²⁴R²⁵;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; substituted —C(O)-polyhydroxyl group; provided that both R⁴⁴ and R⁴⁵ are not H;
n20 is 2 to 26;
n21 is 1 to 26; and
n25 is 1 or 2;

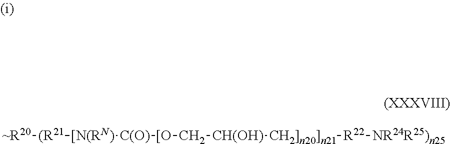

or a stereoisomer or salt thereof, wherein:
R²⁰ is an attachment group to site β or to site R^{b}, or to the enzyme-cleavable group;
R²¹ and R²² are each independently bond or C₁-C₃ alkylene groups;
R^{N} is H or C₁-C₄alkyl;
R²⁴ and R²⁵ are each independently selected from a H; polyhydroxyl group; or substituted polyhydroxyl group, provided that R²⁴ and R²⁵ are not both H;
n20 is 2 to 26;
n21 is 1 to 4; and
n25 is 1, 2 or 3;

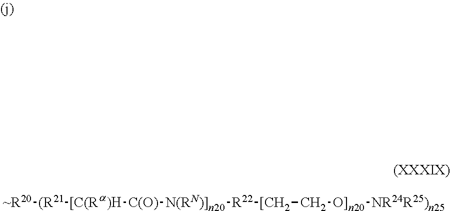

or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, —C₁-C₃alkylene-[O—CH₂—CH₂—]ₙ₂₀, —[CH₂—CH₂—O]ₙ₂₀—C₁-C₃alkylene- or —C₁-C₃alkylene-[O—CH₂—CH₂—]ₙ₂₀—C(O)—;
each R⁰ is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, wherein R²⁸ is the sugar unit of formula (XII) or (XIII); or —NR²⁴R²⁵ together from a C₃-C₈ heterocycle), provided that R²⁴ and R²⁵ are not both H;
each n20 is independently 0 to 26, with the proviso that at least one n20 is 2 to 26; and
n25 is 1 or 2; or

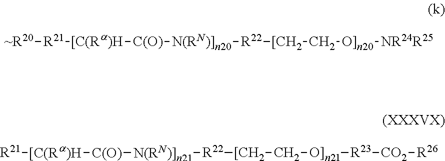

or a stereoisomer or salt thereof, wherein:
R²⁰ is an attachment group to site β or to site R^{b}, or to the enzyme-cleavable group;
R²¹, R²² and R²³ are each, independently, a bond, C₁-C₃ alkylene, —C₁-C₃alkylene-[O—CH₂—CH₂—]ₙ₂₀, —[CH₂—CH₂—O]ₙ₂₀—C₁-C₃alkylene- or —C₁-C₃alkylene-[O—CH₂—CH₂—]ₙ₂₀—C(O)—;
each R⁰ is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is the sugar unit of formula (XII) or (XIII); or —NR²⁴R²⁵ together from a C₃-C₈ heterocycle), provided that R²⁴ and R²⁵ are not both H;
R²⁶ is H or C₁-C₆ alkyl;
each n20 is independently 0 to 26, with the proviso that at least one n20 is 2 to 26; and
each n21 is independently 0 to 26, with the proviso that at least one n21 is 2 to 26.

Embodiment 11. The linker compound of Embodiments 7 or 8, the polar group has a formula selected from the following, or a stereoisomer or salt thereof:

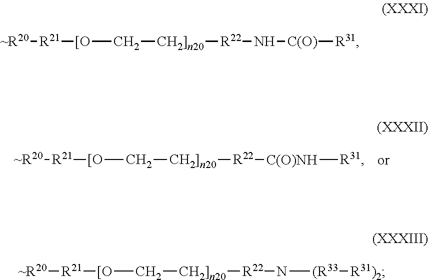

wherein:
R²⁰ is an attachment group to site β or to site R^{b}, or to the amino acid unit, the amino acid unit being an enzyme-cleavable group;
R²¹ and R²² are each, independently, bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, —C₁-C₃ alkylene-C(O), —C(O)—C₁-C₃ alkylene or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy (˜) line indicates an attachment site to R²⁰; and n20 is 2 to 26.

Embodiment 12. The linker compound of Embodiment 7 or 8, the polar group has a formula:

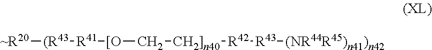

or a stereoisomer or salt thereof, wherein:
R²⁰ is an attachment group to site □, to site R^{b}, or to the amino acid unit, the amino acid unit being an enzyme-cleavable group;
R⁴¹ and R⁴² are each, independently, bond or C₁-C₆ alkylene;
each R⁴³ is, independently, a bond or is selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 2 to 26;
n41 is 1 to 6; and
n42 is 1 to 6.

Embodiment 13. The linker compound of Embodiment 7 or 8, the polar group has a formula:

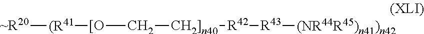

or a stereoisomer or salt thereof, wherein:
R²⁰ is an attachment group to site Q, to site R^{b}, or to the amino acid unit, the amino acid unit being an enzyme-cleavable group;
R⁴¹ and R⁴² are each, independently, bond or C₁-C₆ alkylene;
R⁴³ is a bond or is selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 1 to 26;
n41 is 1 to 6; and
n42 is 1 to 6.

Embodiment 14. The linker compound of Embodiment 7 or 8, the polar group has a formula:

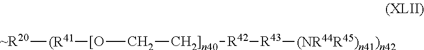

or a stereoisomer or salt thereof, wherein:
R²⁰ is an attachment group to site Q, to site R^{b}, or to the amino acid unit, the amino acid unit being an enzyme-cleavable group;
R⁴¹ and R⁴² are each, independently, bond or C₁-C₃ alkylene;
R⁴³ is a bond or is selected from C₁-C₆ alkylene, —NH—C₁-C₁₂ alkylene, —C₁-C₆ alkylene-NH—, —C(O)—C₁-C₆ alkylene, —C₁-C₆ alkylene-C(O)—, —NH—C₁-C₆ alkylene-C(O)—, —C(O)—C₁-C₆ alkylene-NH—, —NH—C(O)—NH—, —NH—C(O)—, —NH—C(O)—C₁-C₆ alkylene, —C(O)—NH—C₁-C₁₂ alkylene, -heteroarylene, heteroaryl-C₁-C₆ alkylene, heteroaryl-C₁-C₆ alkylene-C(O)—, or —C(O)NR⁴⁶R⁴⁷, wherein one of R⁴⁶ and R⁴⁷ is H or C₁-C₆ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 1 to 16;
n41 is 1 to 4; and
n42 is 1 to 4.

Embodiment 15. The linker compound of Embodiments 7 or 8, the polar group has a formula selected from:

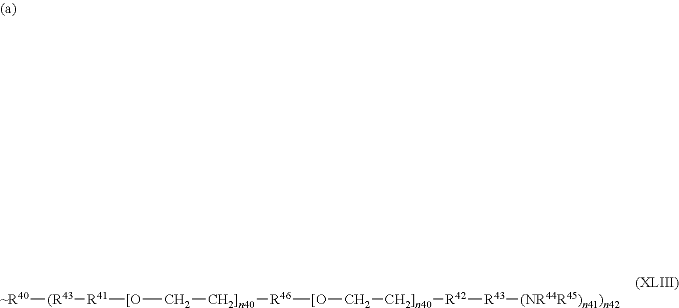

or a stereoisomer or salt thereof, wherein:
R⁴⁰ is an attachment group to site R^{b}, or to the amino acid unit, the amino acid unit being an enzyme-cleavable group;
R⁴¹ and R⁴² are each, independently, a bond or C₁-C₆ alkylene;
each R⁴³ is, independently, selected from a bond, C₁-C₁₂ alkylene, —OC₁-C₁₂ alkylene, —C(═O)—, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, C₁-C₁₂alkylene-NH—C(O)—, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
each R⁴⁶ is independently selected from —NR⁵⁰—, —NR⁵⁰—C₁-C₆alkylene-NR⁵⁰—, —NR⁵⁰—C(O)—NR⁵⁰—S(O)₂—NR⁵⁰— or —NR⁵⁰—C(O)—C₁₋₆alkylene-;
each R⁵⁰ is independently selected from H, C₁-C₆ alkyl, or polyhydroxyl group;
each n40 is independently 2 to 26;
n41 is 1 to 6; and
n42 is 1 to 6;

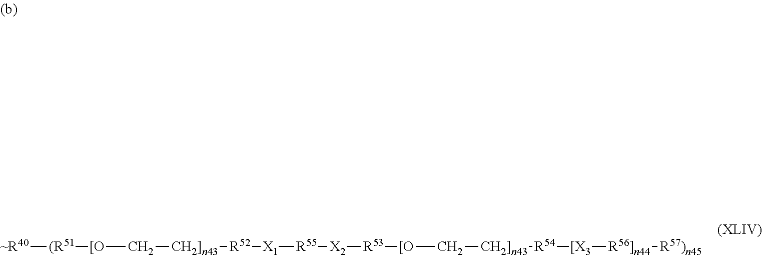

or a stereoisomer or salt thereof, wherein:
R⁴⁰ is an attachment group to site R^{b}, or to the enzyme-cleavable group;
R⁵¹, R⁵², R⁵³ and R⁵⁴ are each, independently, a bond or C₁-C₆ alkylene; X₁, X₂ and X₃ are each independently —NR^{N}—C(O)— or —C(O)—NR^{N}—;
each R^{N} independently represent H, C₁-C₆ alkyl, or polyhydroxyl group;
R⁵⁵ and R⁵⁶ each independently represent a bivalent polyhydroxyl group;
R⁵⁷ is H, OH or C₁-C₆ alkyl;
each n43 is independently 0 to 26, with the proviso that at least one n43 is 1 to 26;
n44 is 0 to 10; and
n45 is 1 or 2; or

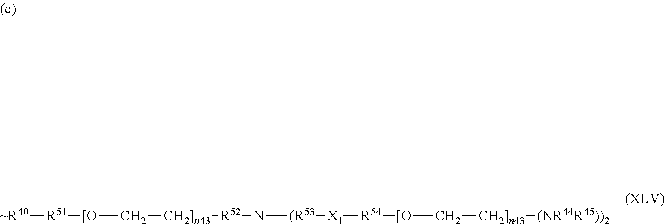

or a stereoisomer or salt thereof, wherein:
R⁴⁰ is an attachment group to site R^{b}, or to the enzyme-cleavable group;
R⁵¹, R⁵³ and R⁵⁴ are each, independently, a bond or optionally-substituted C₁-C₆ alkylene;
R⁵² is a bond, C₁-C₆ alkylene, —C(O)— or —O—C(O)—; each X₁ is independently —NR^{N}—C(O)— or —C(O)—NR^{N}—;
each R^{N} independently represent H, C₁-C₆ alkyl, or polyhydroxyl group;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; and
each n43 is independently 2 to 26.

Embodiment 16. The linker compound of Embodiment 52, the polar group has one of the following structures prior to attachment to the enzyme-cleavable group and/or to the linker unit:

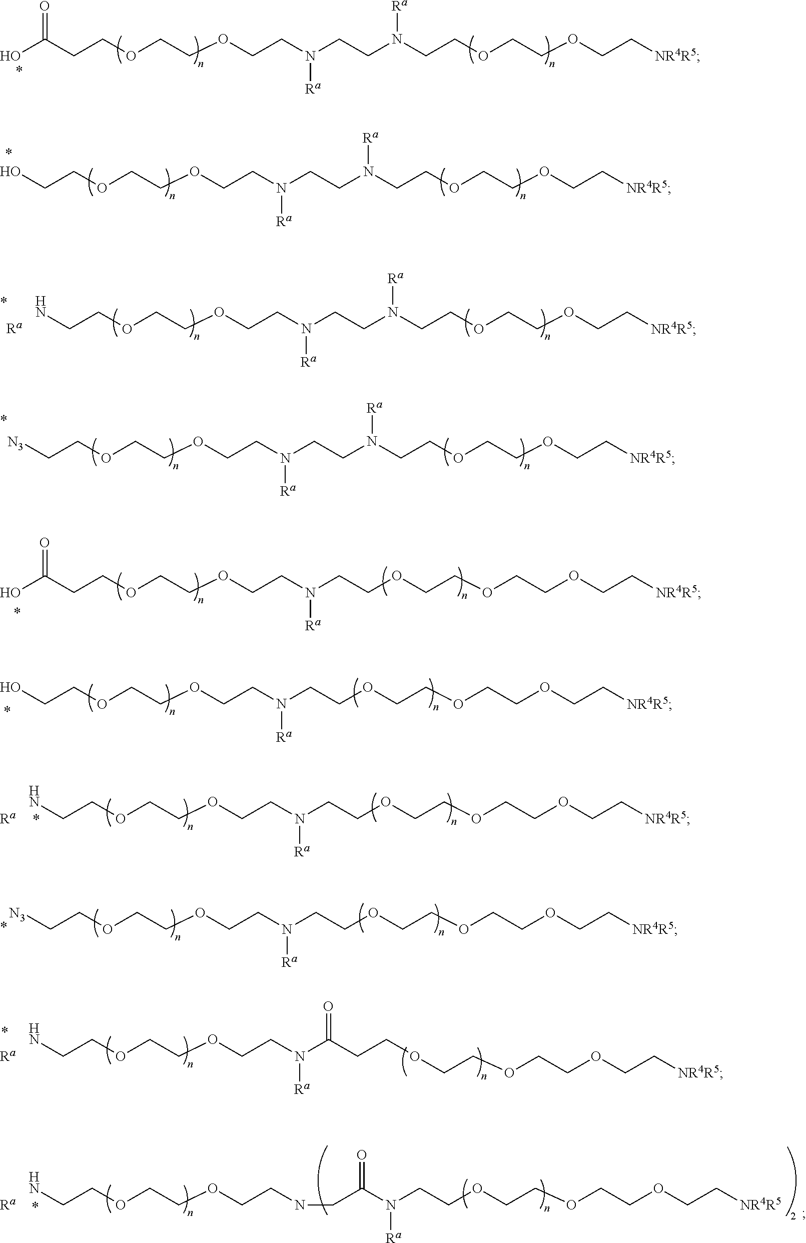

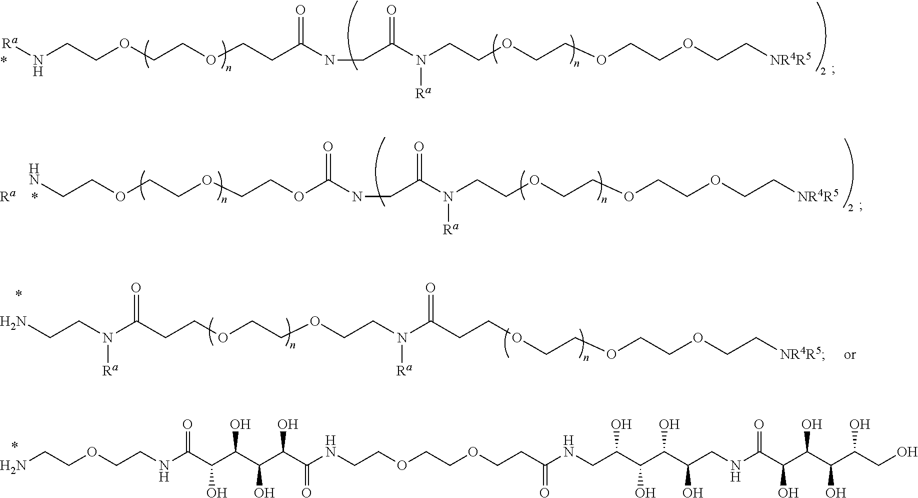

wherein:
(*) indicates the attachment site to site R^{b}, or to the enzyme-cleavable group;
each R is independently H, alkyl or polyhydroxyl group;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; and each n is independently 1 to 12.

Embodiment 17. The linker compound of Embodiment 7 or 8, the polar group has a formula selected from:

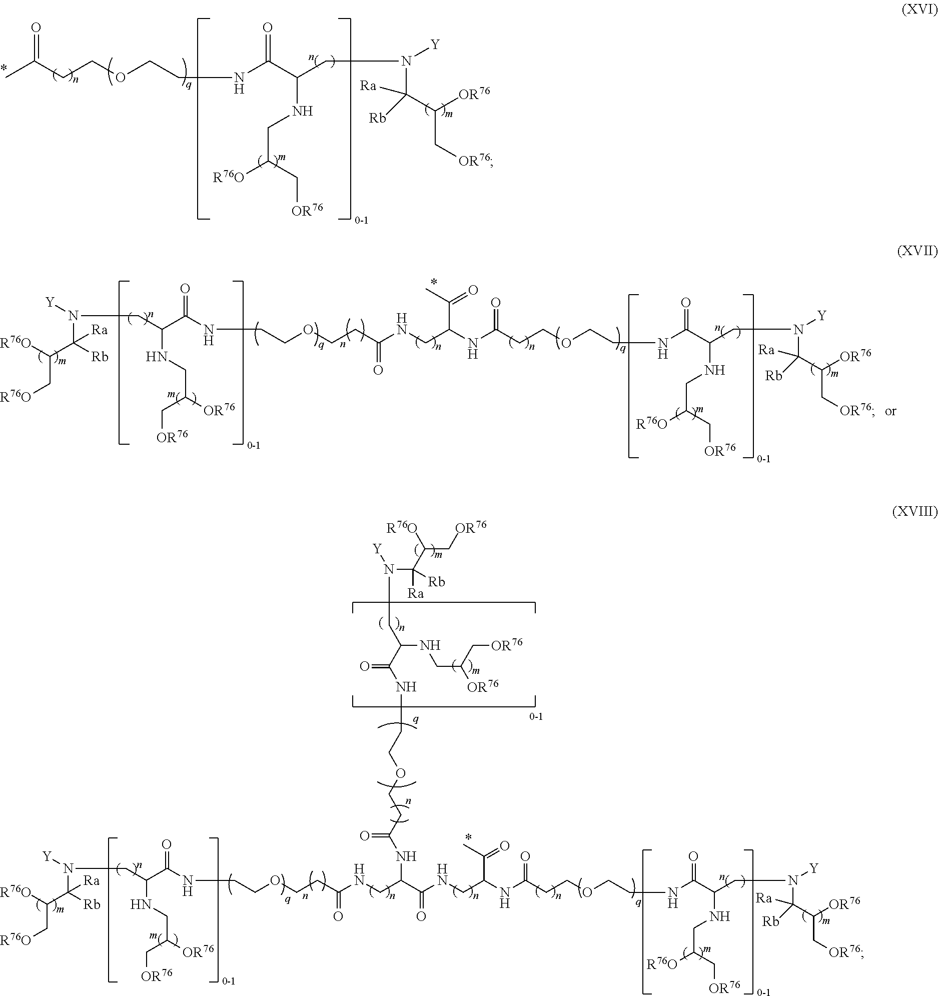

or a stereoisomer or salt thereof, wherein:
each Y is independently R⁷⁶ or

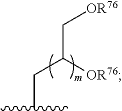

each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 2-26;
each m is independently 1 to 4;
each n is independently 1 to 4;
each v is independently 1 to 6; and
each * is an attachment site to R^{b}, or to the amino acid unit, the amino acid unit being an enzyme-cleavable group.

Embodiment 18. The linker compound of Embodiment 7 or 8, wherein the polar group comprises at least one carboxyl unit having the following formula:

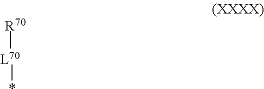

or a stereoisomer or salt thereof, wherein:
(a)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the amino acid unit, the amino acid unit being an enzyme-cleavable group, or to a remainder of the polar group;
R⁷⁰ is —NR⁷¹(R⁷²-R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is a bond or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(b)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the polar group;
R⁷⁰ is —NR⁷¹(R⁷⁵—(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(c)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the polar group;
R⁷⁰ is —N(R⁷⁴-R⁷³)(R⁷²-R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide.

Embodiment 19. The linker compound of Embodiment 18, comprising a formula selected from the following:

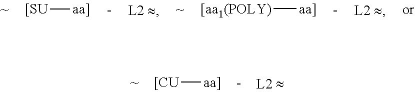

wherein the square brackets indicate the amino acid unit, each aa is an optional subunit of the amino acid unit, L2 is the linker unit, each wavy line (˜) indicates an attachment site for a stretcher group; aa₁(POLY) is a polymer unit attached to the subunit of the amino acid unit, SU is the sugar unit attached to the subunit of the amino acid unit or to the linker unit, and CU is the carboxyl unit attached to the subunit of the amino acid unit or to the linker unit; and the double wavy (≈) line indicates an attachment site for the drug units, wherein aa and aa₁ are independently selected from alpha, beta and gamma amino acids and derivatives thereof.

Embodiment 20. The linker compound of Embodiments 18, comprising a formula selected from the following:

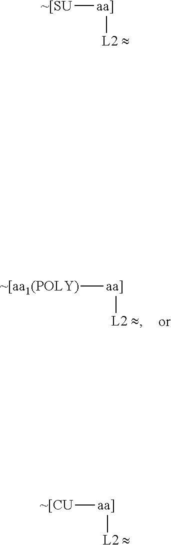

wherein square brackets indicate the amino acid unit, each aa is a subunit of the amino acid unit, L2 is the linker unit attached to a side chain of aa, the wavy line (˜) indicates an attachment site for a stretcher group; aa₁(POLY) is a polymer unit attached to aa, SU is the sugar unit attached to aa, CU is the carboxyl unit attached to aa, and the double wavy (≈) line indicates an attachment site for the drug unit; wherein aa and aa₁ are independently selected from alpha, beta and gamma amino acids and derivatives thereof.

Embodiment 21. The linker compound of Embodiment 18, comprising a formula selected from the following:

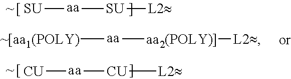

wherein square brackets indicate the amino acid unit, aa is an optional subunit of the amino acid unit, L2 is the linker unit, the wavy line (˜) indicates an attachment site for a stretcher group; each of aa₁(POLY) and aa₂(POLY) is a polymer unit attached to aa or to other polymer unit; each SU is the sugar unit attached to aa or other sugar unit, each CU is the carboxyl unit attached to aa or to other carboxyl unit, and the double wavy (≈) line indicates an attachment site for the drug unit; wherein aa, aa₁ and aa₂ are independently selected from alpha, beta and gamma amino acids and derivatives thereof.

Embodiment 22. The linker compound of Embodiment 18, comprising a formula selected from the following:

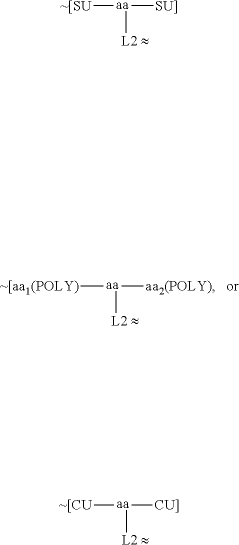

wherein square brackets indicate the amino acid unit, aa is a subunit of the amino acid unit, L2 is the linker unit attached to a side chain of aa, each wavy line (˜) indicates an attachment site for a stretcher group; each of aa₁(POLY) and aa₂(POLY) is a polymer unit attached to aa, each SU is the sugar unit attached to aa; each CU is the carboxyl unit attached to aa; and the double wavy (≈) line indicates an attachment site for the drug units; wherein each of aa, aa₁ and aa₂ is independently selected from alpha, beta and gamma amino acids and derivatives thereof.

Embodiment 23. The linker compound of any one of Embodiments 1-3, 5, 7-22, wherein the linker unit has the following structure:

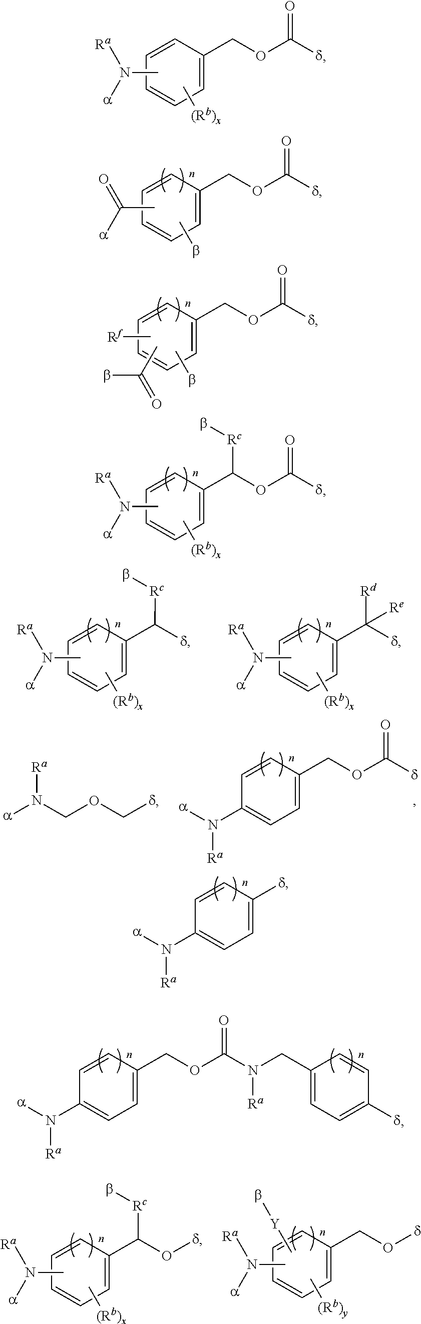

or a stereoisomer or salt thereof, wherein:
α—represents an attachment site to the amino acid unit, when present, or to the stretcher unit;
β—represents an attachment site to the at least one polar group;
δ—represents an attachment site to the drug unit or an attachment site to a linking group attached to the drug unit;
R^{a} is H or C₁₋₆ alkyl;
R^{b} is H, halo, C₁₋₆ alkyl, an attachment site to the drug unit or the linking group attached to the drug unit, or an attachment site to the at least one polar group or a linking group attached to the at least one polar group;
R^{c} is a bond, —C(O)—, —S(O)—, —SO₂—, C₁₋₆ alkylene, C₁₋₆ alkynylene, triazolyl, or combinations thereof;
R^{e} and R^{d} are each independently H, C₁-C₆ alkyl, C₁₋₆ alkylene, C₁₋₆ alkynylene, nitrile group, alkynyl group, nitrogen triyl group, an ester group, substituted triazolyl, substituted amino group, substituted thiol group, substituted silicon group, substituted phosphate group, 3 to 8 aromatic rings, cyclic hydrocarbons, cyclic hydrocarbon heterocycles, aromatic heterocycles, or R^{e} and R^{d} join together to form cycloalkyl, or an attachment site to a linking group attached to the at least one polar group;
R^{f} is an attachment site to the stretcher unit or an attachment site to a linking group attached to the stretcher unit;
Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl, or combinations thereof;
x is 0, 1, 2, 3 or 4;
y is 0, 1, 2 or 3;
   n is 0-2.

Embodiment 24. The linker compound of any one of Embodiments 1-23, wherein the stretcher group is selected from the following:

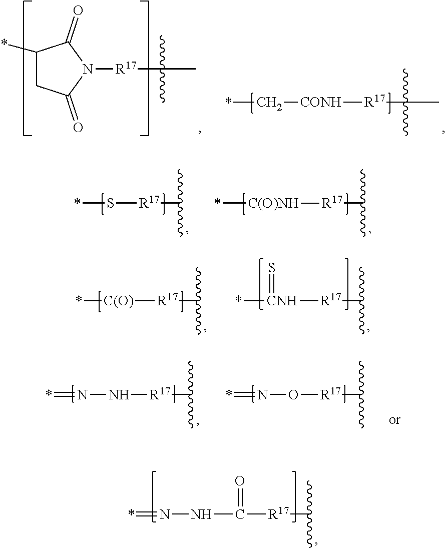

wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)b-C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀ alkylene-C(O)NH—C₁-C₈alkylene-[O—CH₂—CH₂]ₙ—C(O)— (where n is 1 to 26), C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
   wherein the stretcher group comprises maleimido(C₁-C₁₀ alkylene-C(O)—, maleimido(CH₂OCH₂)ₚ₂(C₁-C₁₀ alkylene)C(O)—, maleimido(C₁-C₁₀ alkylene) (CH₂OCH₂)ₚ₂C(O)—, or a ring open form thereof, wherein p2 is from 1 to 26;
   and wherein * is an attachment to the EGFR and c-MET binding agent, and the wavy line is an attachment to the amino acid unit, the amino acid unit being an enzyme-cleavable group.

Embodiment 25. The linker compound of Embodiment 24, wherein the stretcher group is selected from the following:

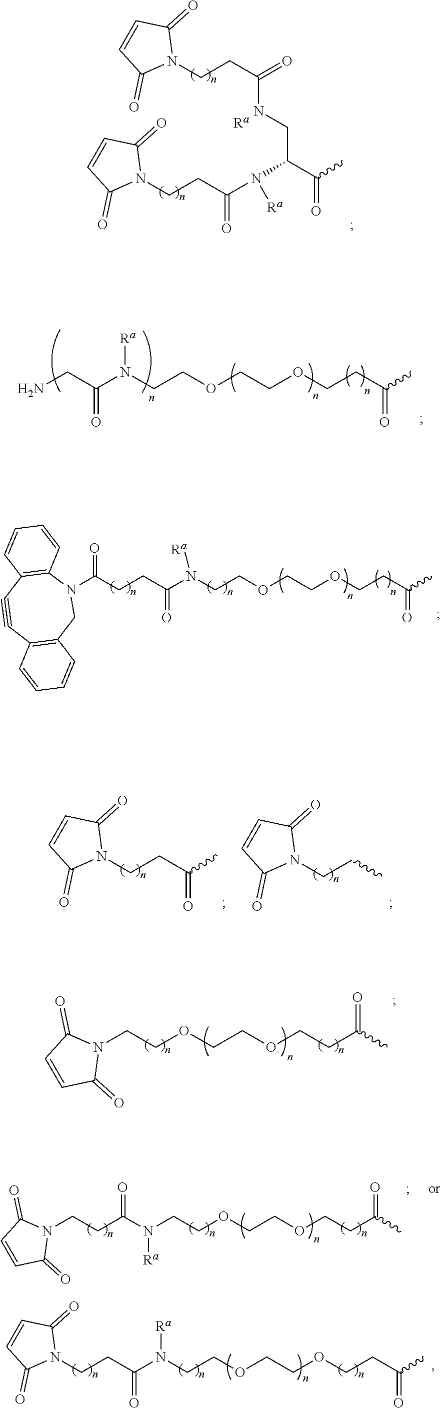

or a stereoisomer thereof, wherein each R^{a} is independently H or C₁₋₆ alkyl, each n is independently 0-12, and the wavy line [symbol] indicates an attachment site of the stretcher group to the amino acid unit, and the attachment site for the targeting unit is on a maleimide, primary amine or alkyne functional group.

Embodiment 26. The linker compound of any one of Embodiments 1-25, having one of the following structures:

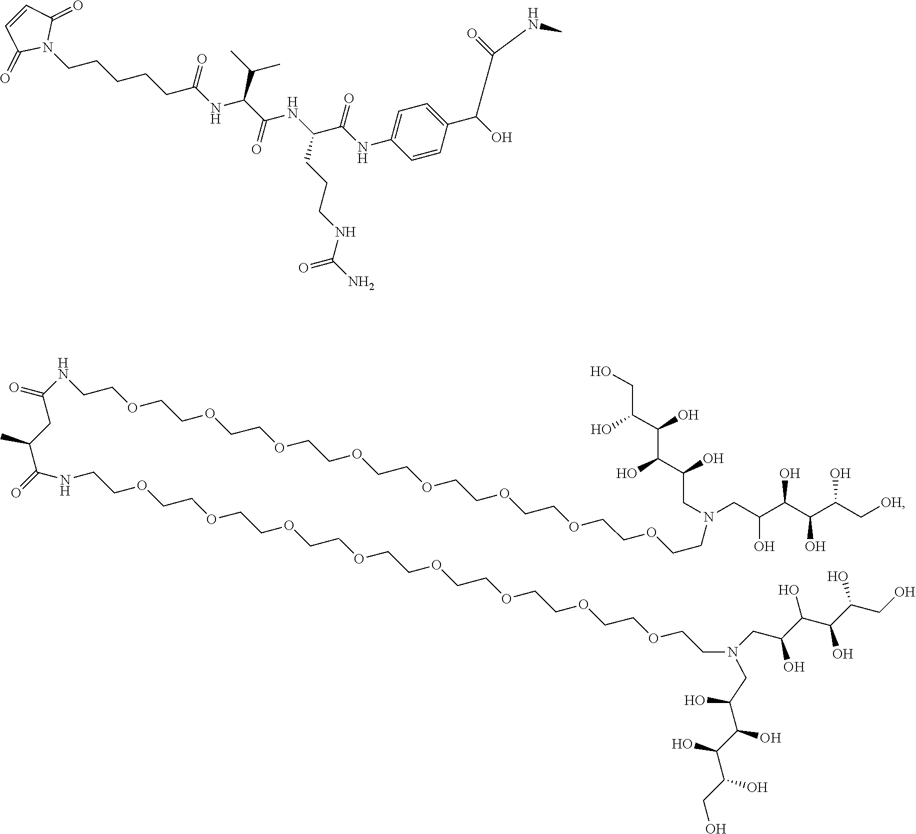

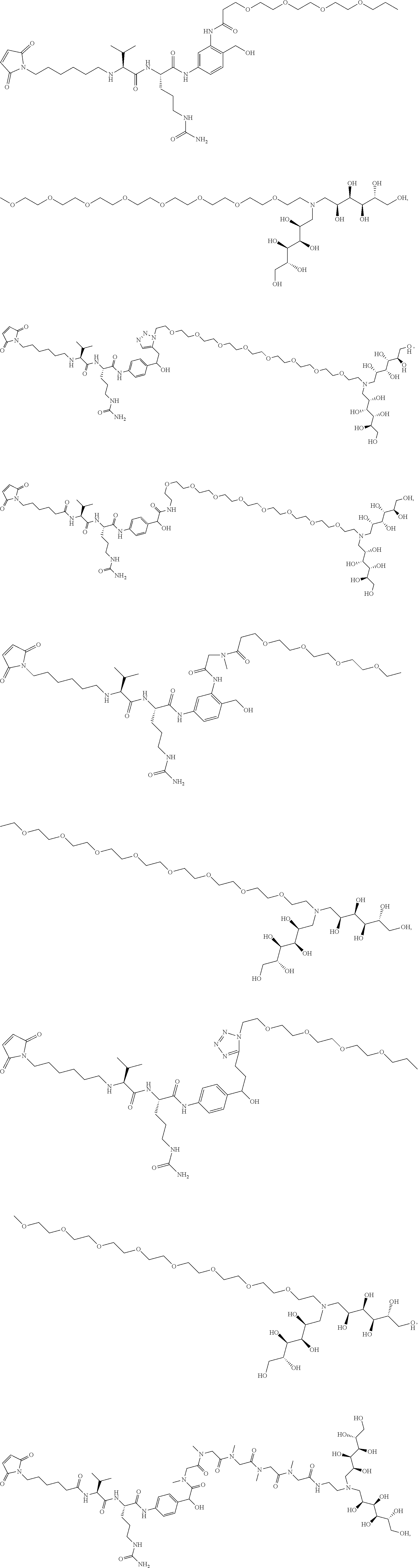

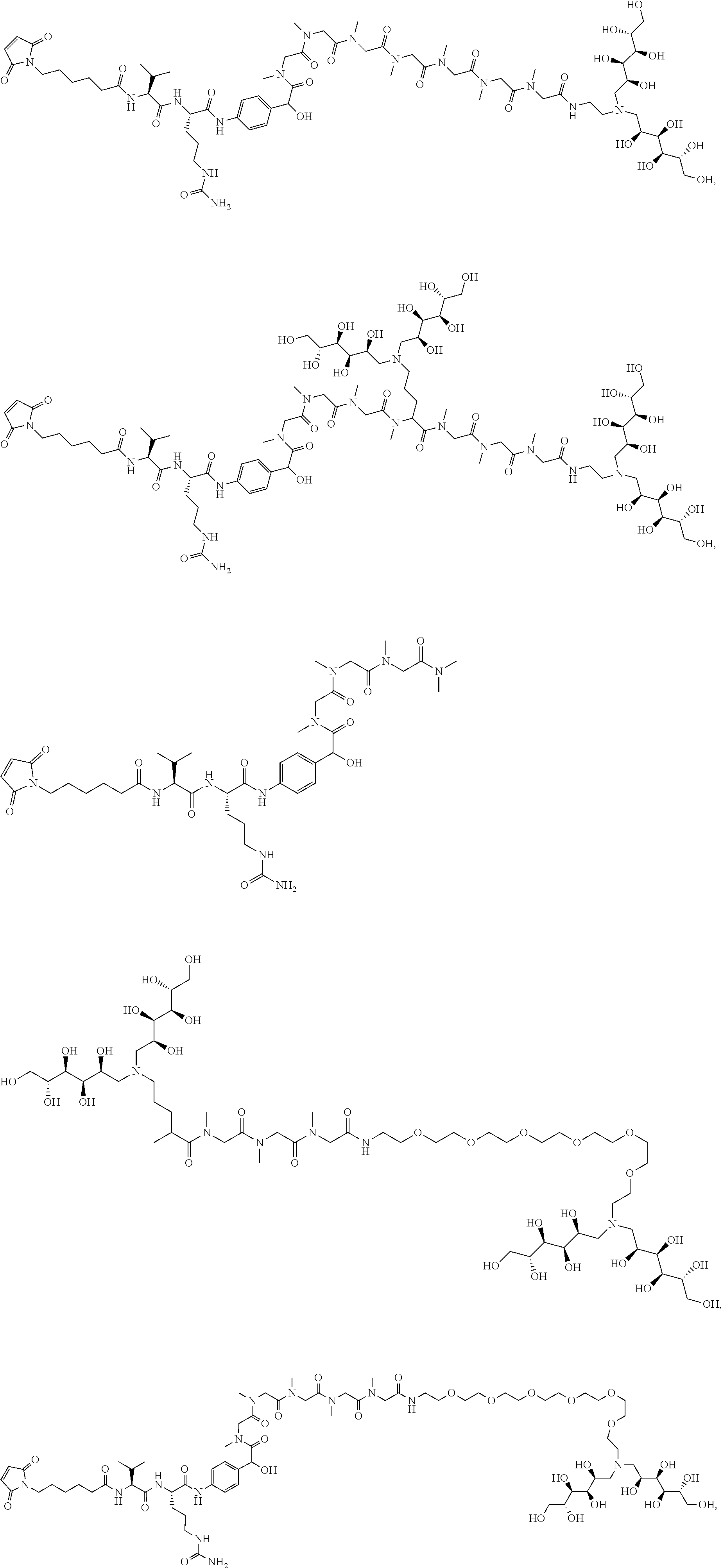

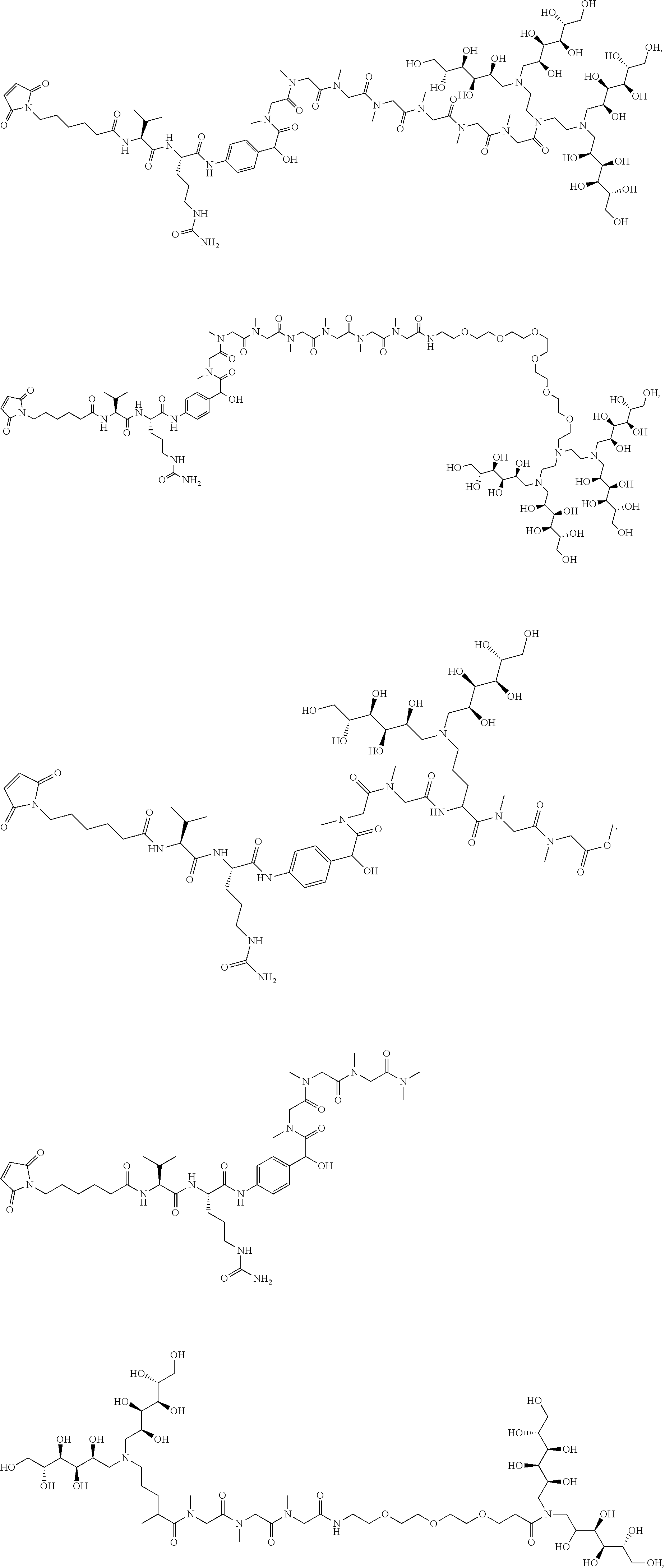

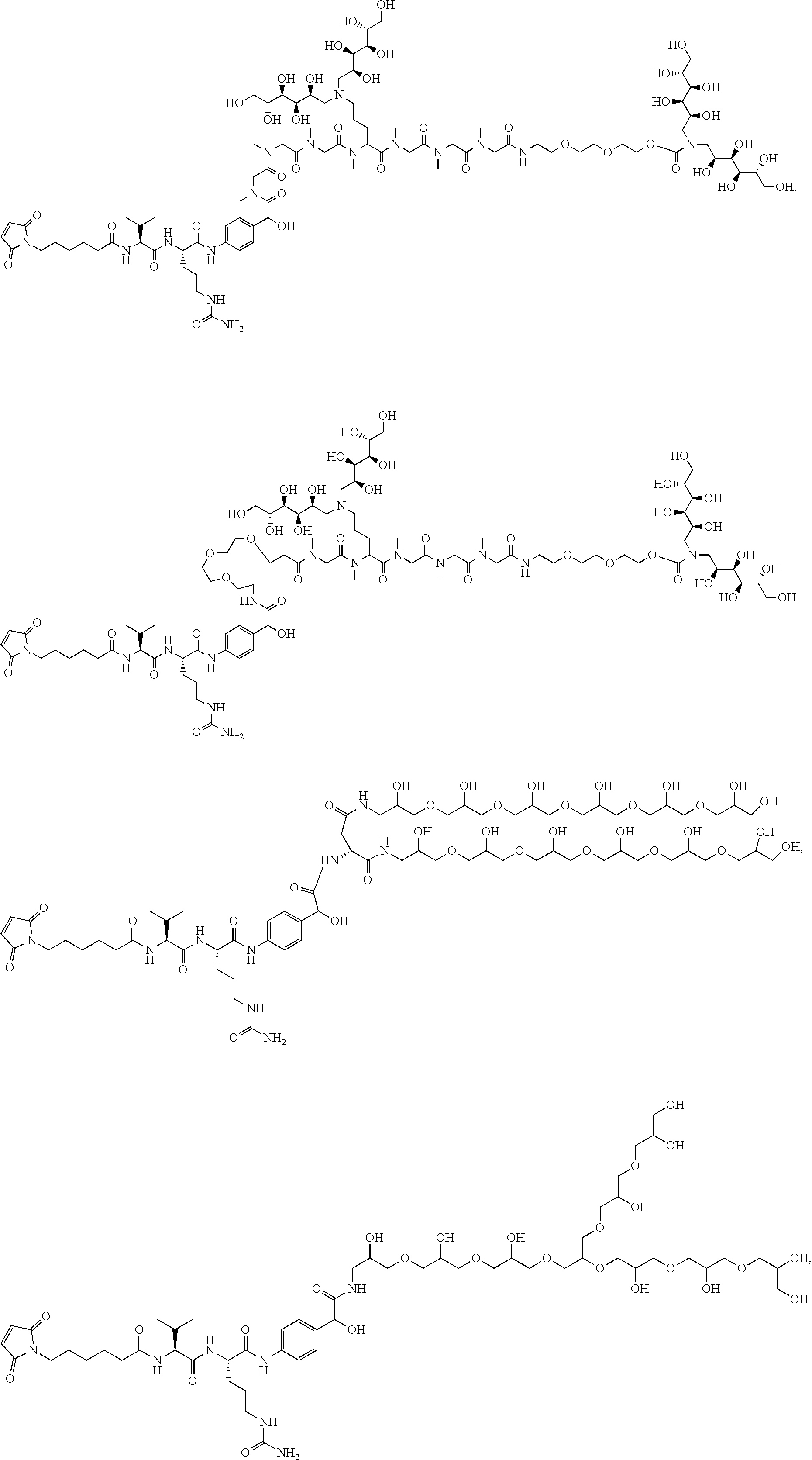

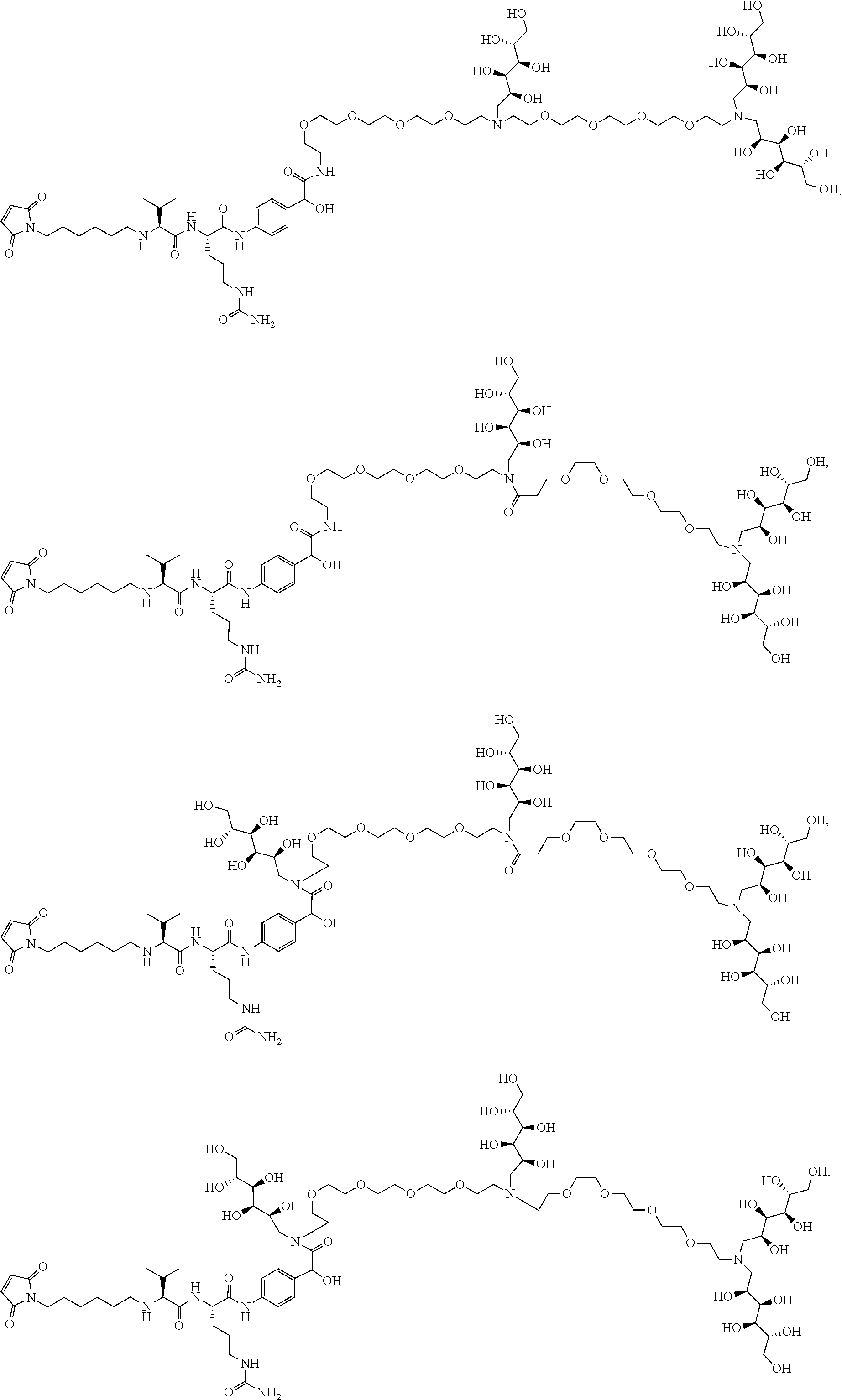

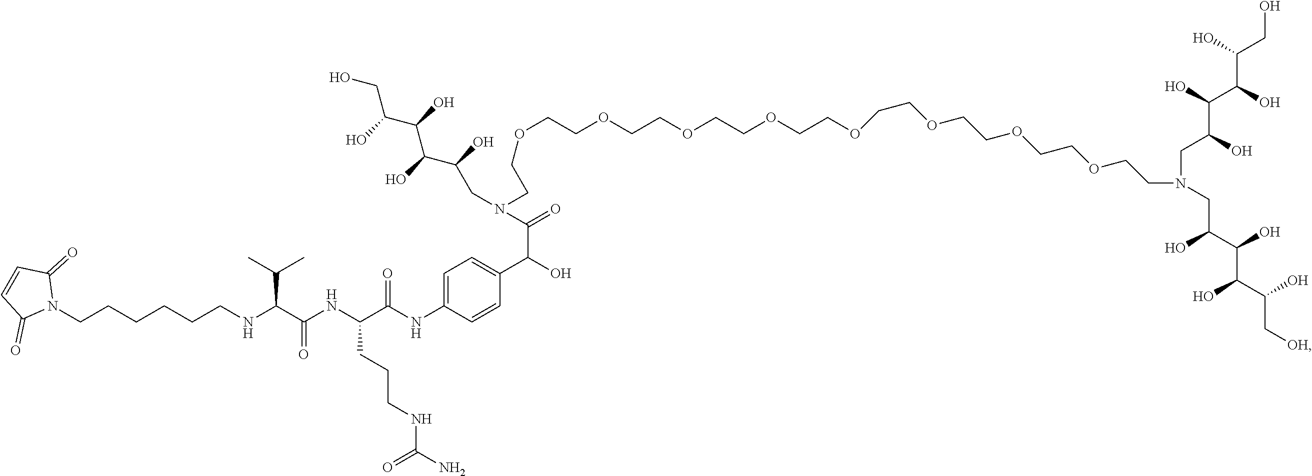 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1]

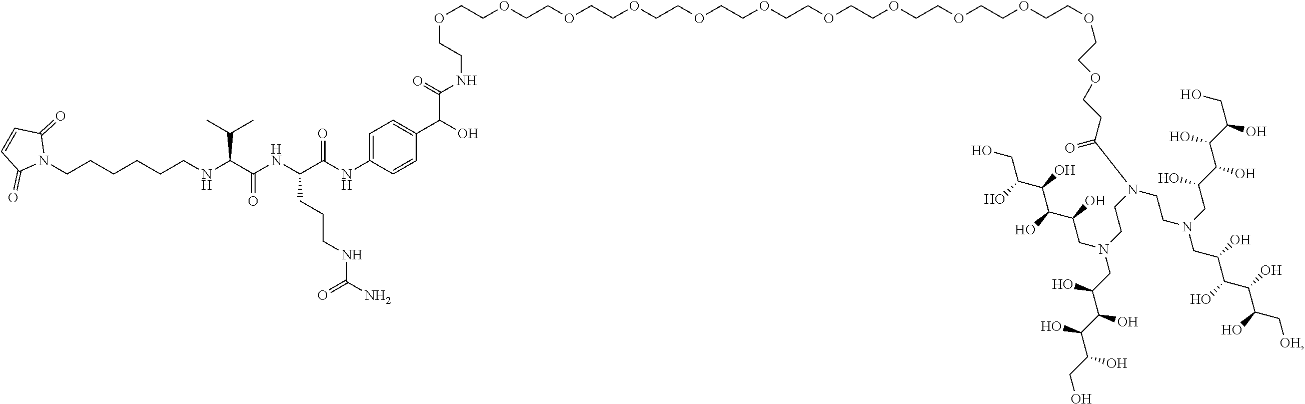 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCOCCOCCOCCOCCOCCOCCOCCOCCOCCOCCC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)cc1]

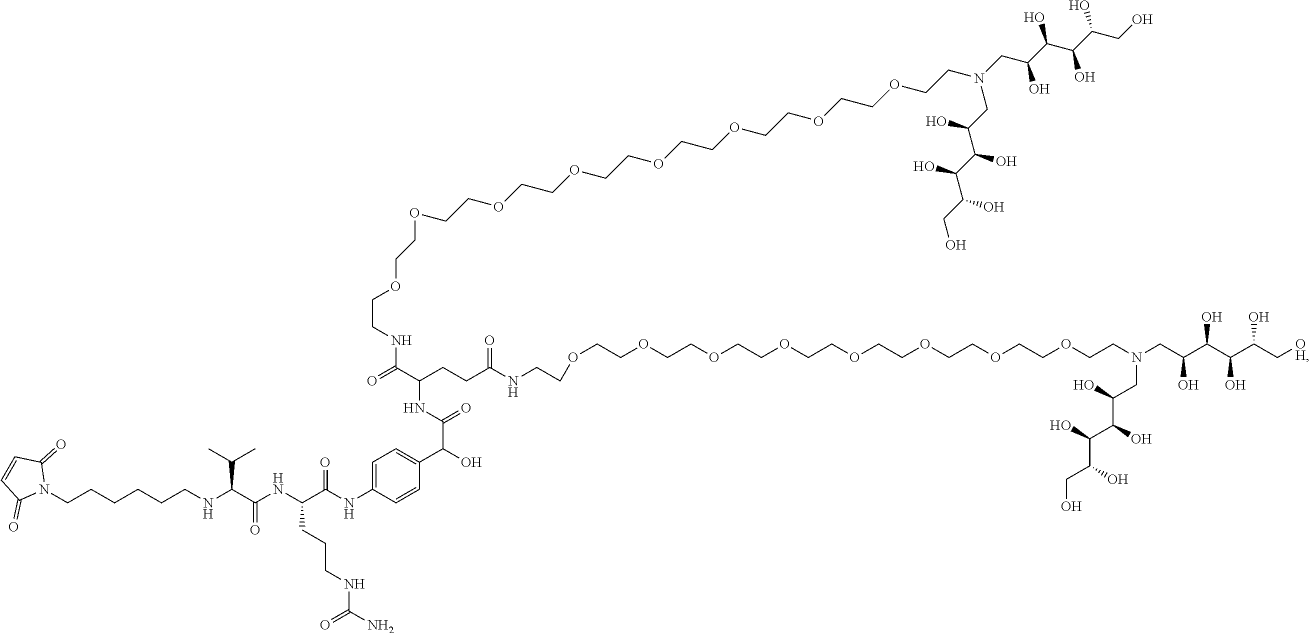 [SMILES: CC(C)[C@H](NCCCCCCN1C(=O)C=CC1=O)C(=O)N[C@@H](CCCNC(N)=O)C(=O)Nc1ccc(C(O)C(=O)NC(CCC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

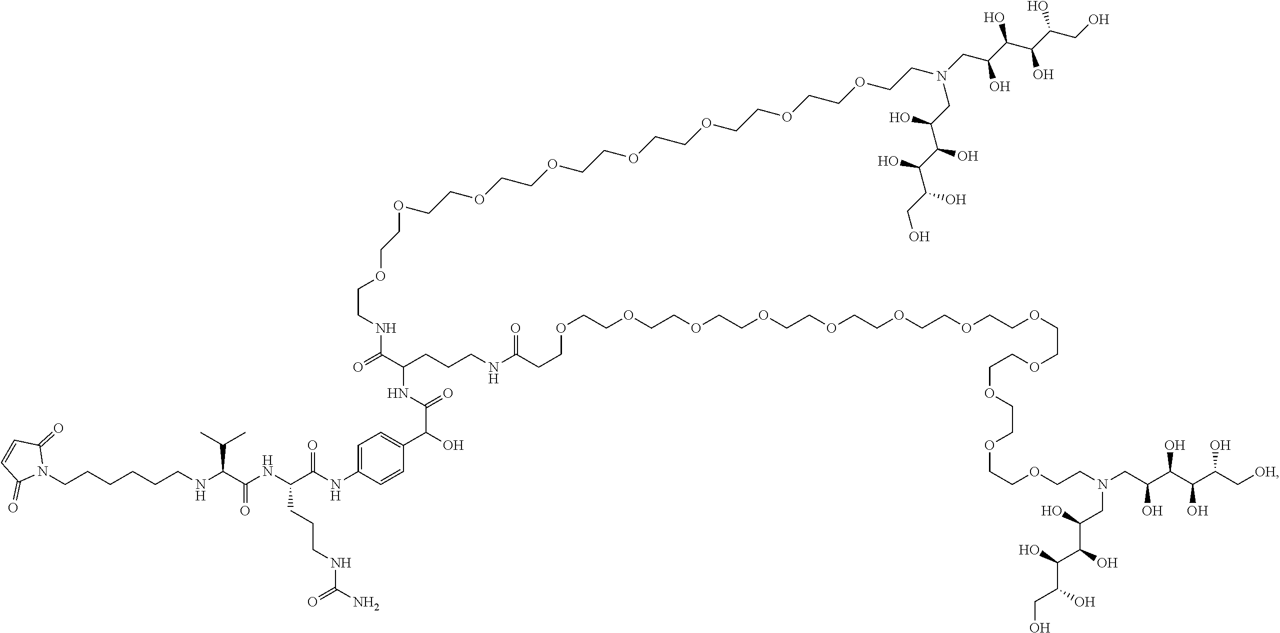 [SMILES: CC(C)[C@H](NCCCCCCN1C(=O)C=CC1=O)C(=O)N[C@@H](CCCNC(N)=O)C(=O)Nc1ccc(C(O)C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

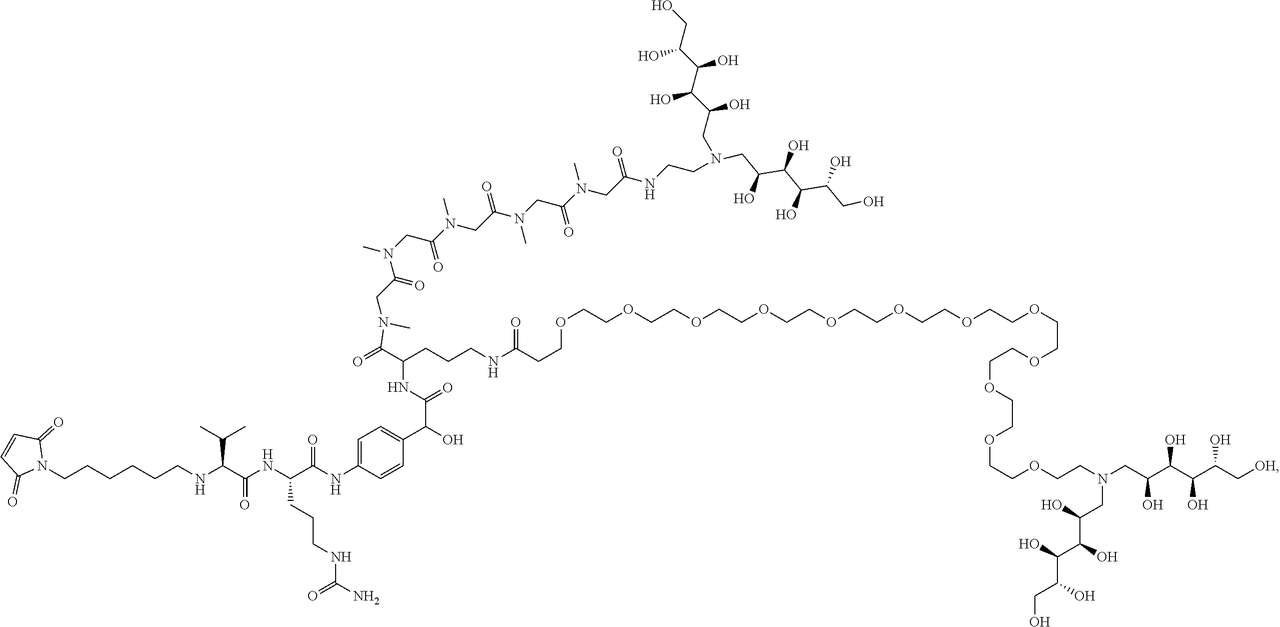 [SMILES: CC(C)[C@H](NCCCCCCN1C(=O)C=CC1=O)C(=O)N[C@@H](CCCNC(N)=O)C(=O)Nc1ccc(C(O)C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1]

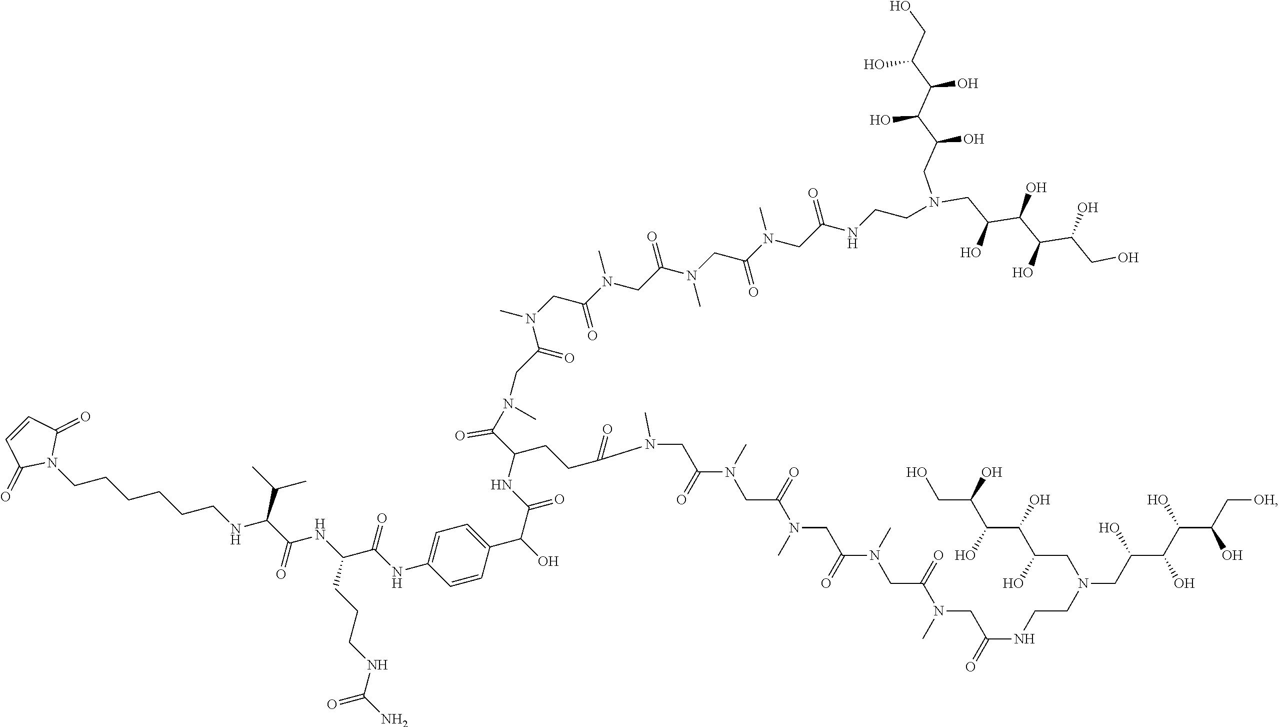 [SMILES: CC(C)[C@H](NCCCCCCN1C(=O)C=CC1=O)C(=O)N[C@@H](CCCNC(N)=O)C(=O)Nc1ccc(C(O)C(=O)NC(CCC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1]

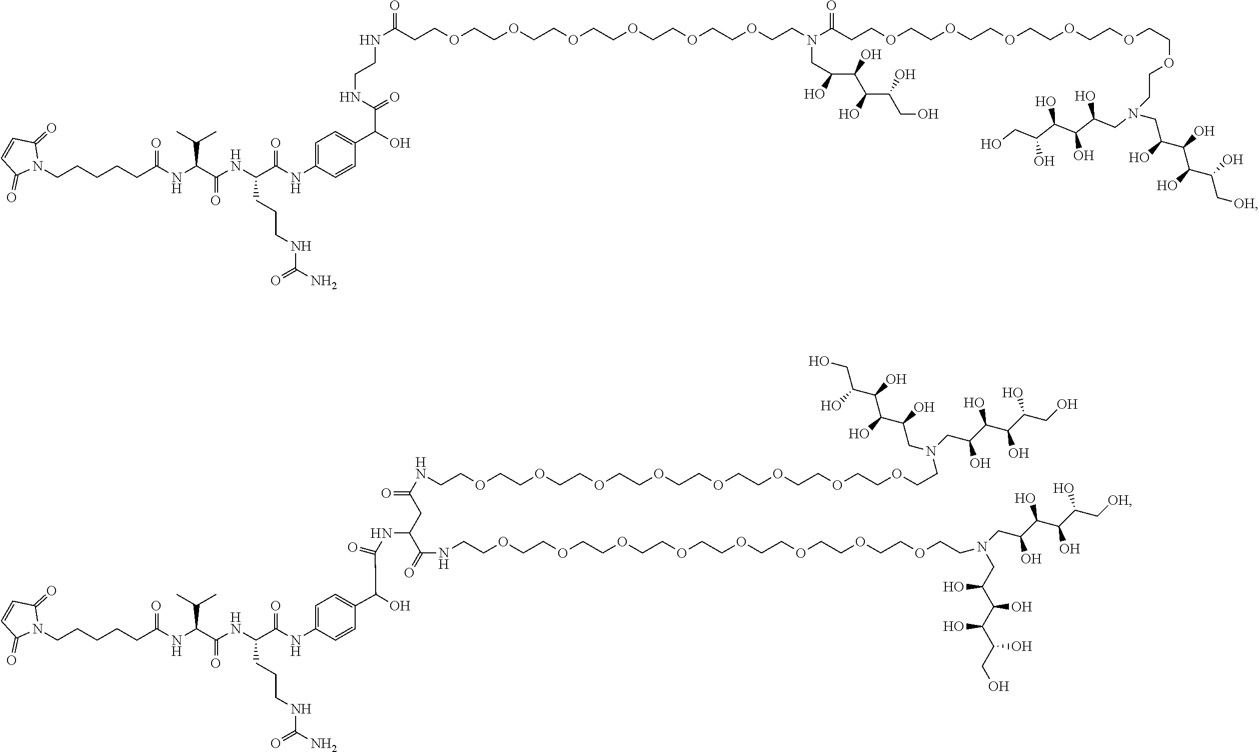

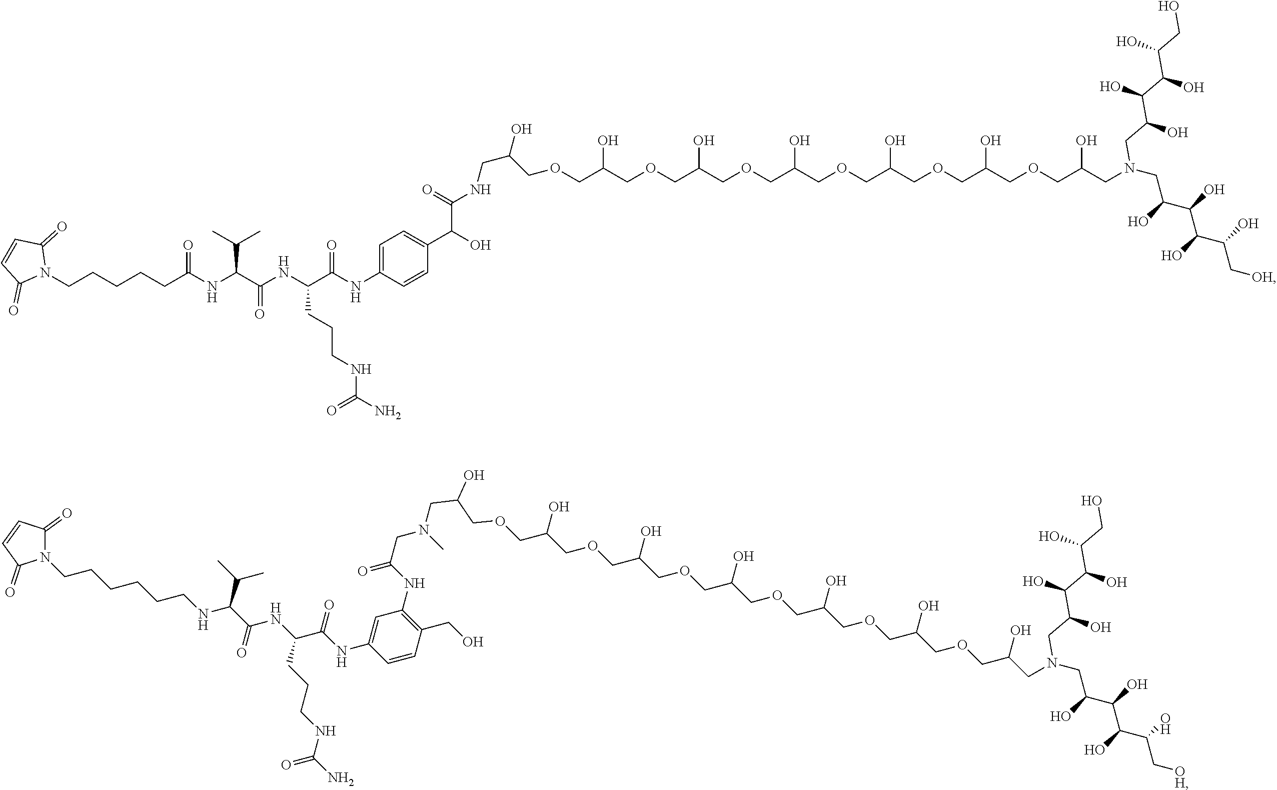 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1 ; CC(C)[C@H](NCCCCCCN1C(=O)C=CC1=O)C(=O)N[C@@H](CCCNC(N)=O)C(=O)Nc1ccc(CO)c(NC(=O)CN(C)CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1]

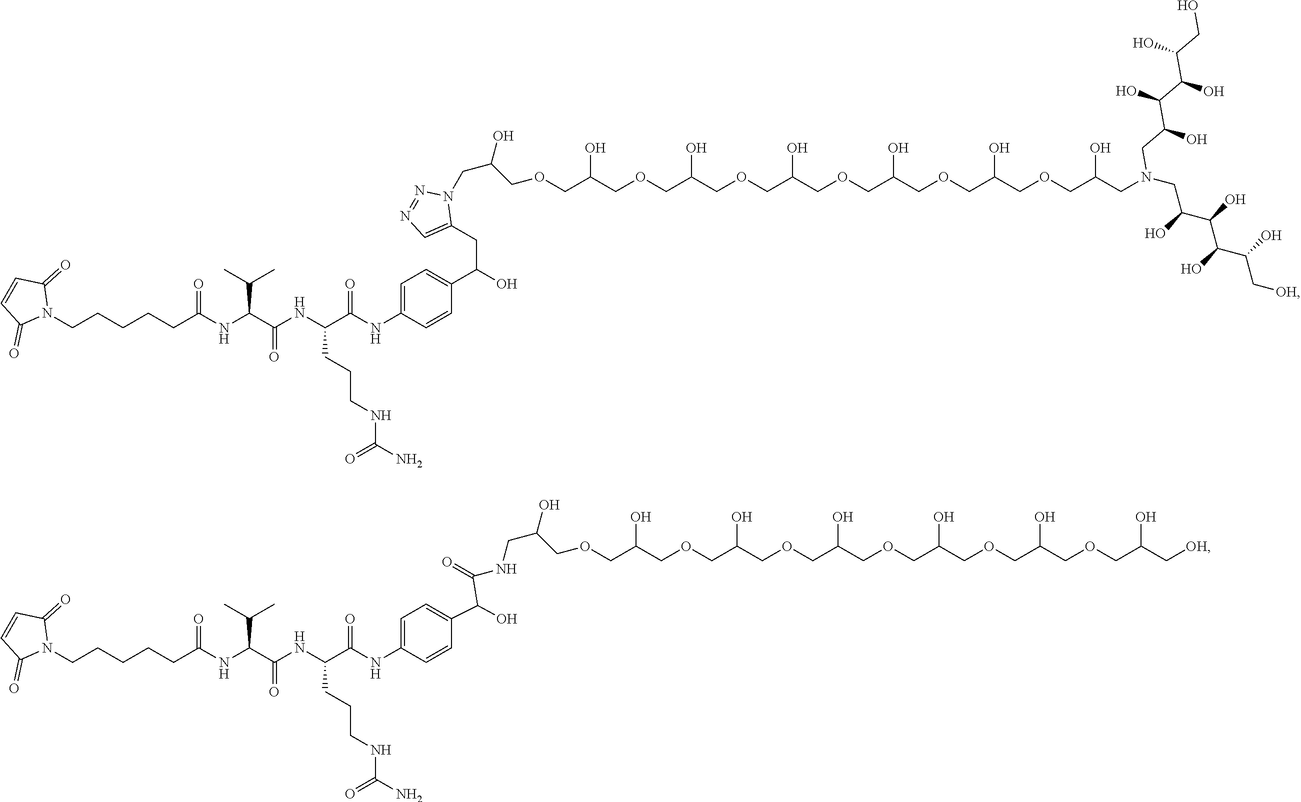 [SMILES: CC(C)[C@H](NC(=O)CCCCCN1C(=O)C=CC1=O)C(=O)N[C@@H](CCCNC(N)=O)C(=O)Nc1ccc(C(O)Cc2cnnn2CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCC(O)CO)cc1]

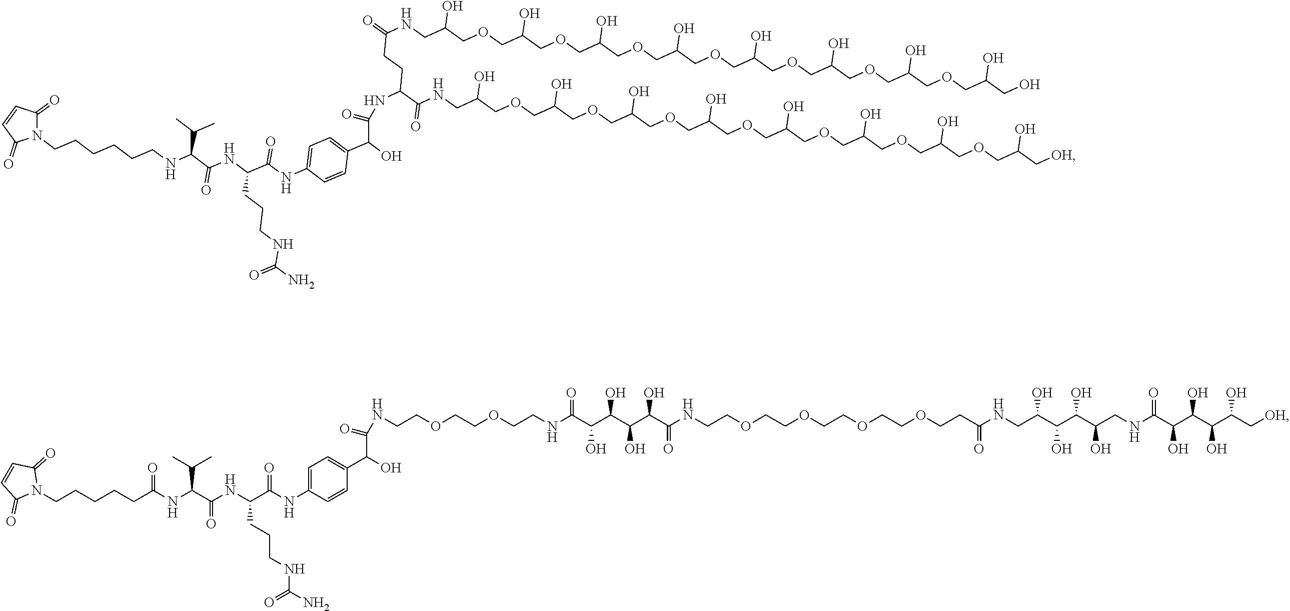 [SMILES: CC(C)[C@H](NCCCCCCN1C(=O)C=CC1=O)C(=O)N[C@@H](CCCNC(N)=O)C(=O)Nc1ccc(C(O)C(=O)NC(CCC(=O)NCC(O)COCC(O)COCC(O)COCC(O)COCC(O)COCC(O)COCC(O)COCC(O)CO)C(=O)NCC(O)COCC(O)COCC(O)COCC(O)COCC(O)COCC(O)COCC(O)COCC(O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCNC(=O)[C@@H](O)[C@@H](O)[C@H](O)[C@@H](O)C(=O)NCCOCCOCCOCCOCCC(=O)NC[C@H](O)[C@@H](O)[C@H](O)[C@H](O)CNC(=O)[C@H](O)[C@@H](O)[C@H](O)[C@H](O)CO)cc1]

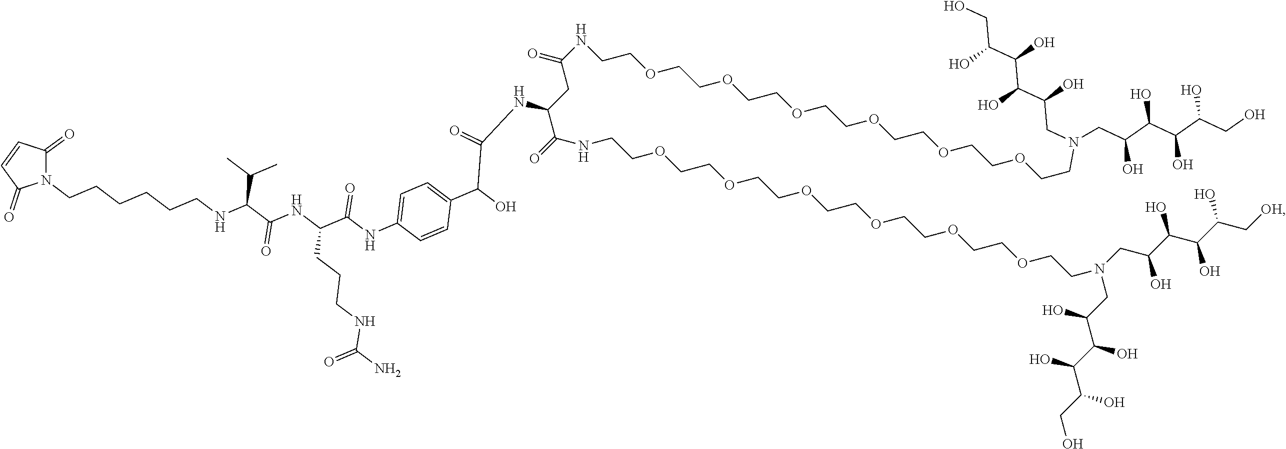 [SMILES: CC(C)[C@H](NCCCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

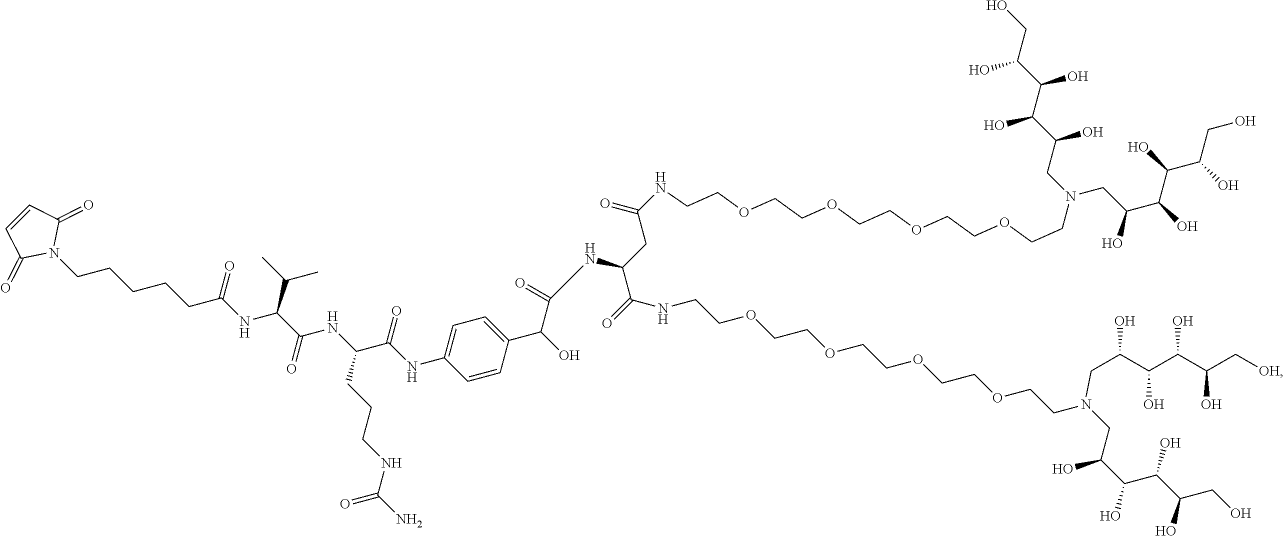 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCN(C[C@H](O)[C@H](O)[C@@H](O)[C@@H](O)CO)C[C@H](O)[C@@H](O)[C@H](O)[C@H](O)CO)C(=O)NCCOCCOCCOCCOCCN(C[C@H](O)[C@@H](O)[C@H](O)[C@H](O)CO)C[C@H](O)[C@@H](O)[C@H](O)[C@H](O)CO)cc1]

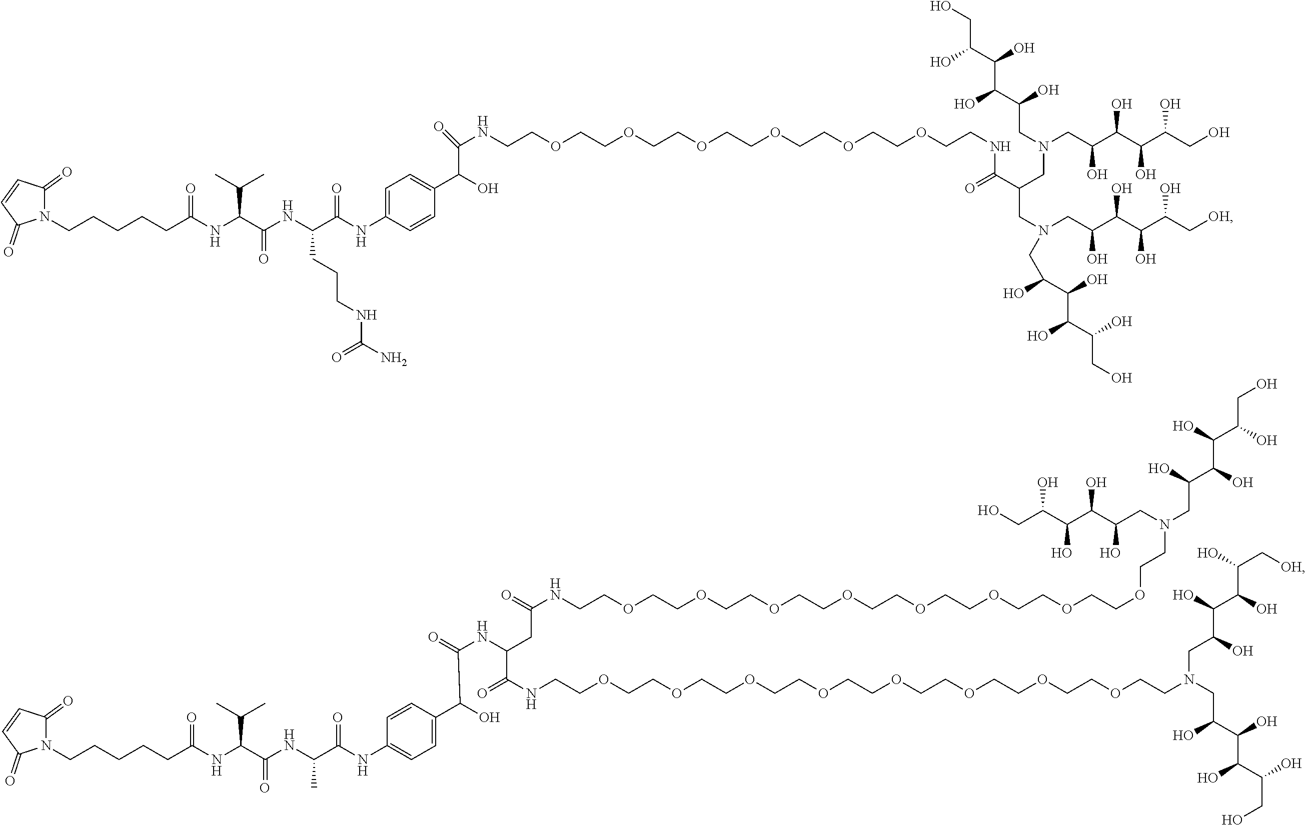

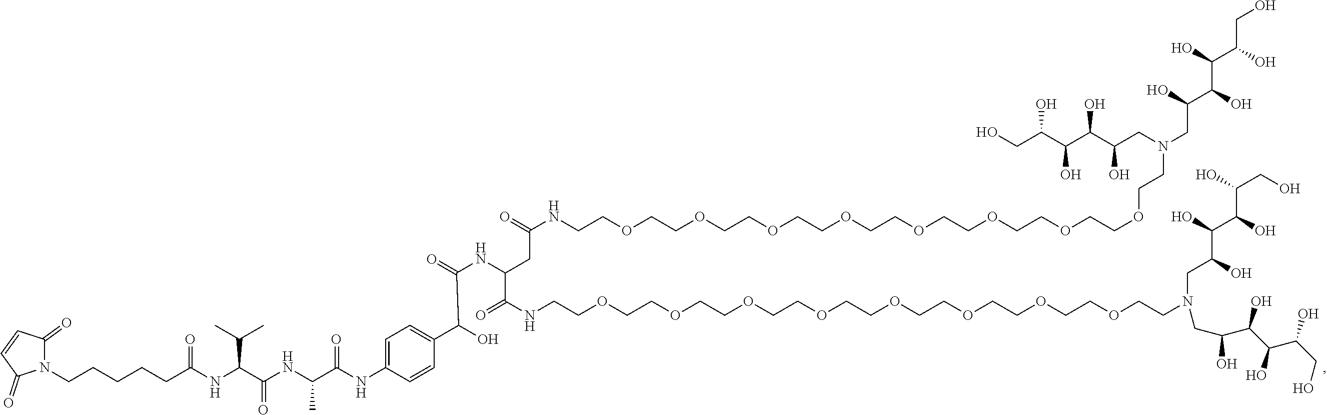 [SMILES: CC(C)[C@H](NC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

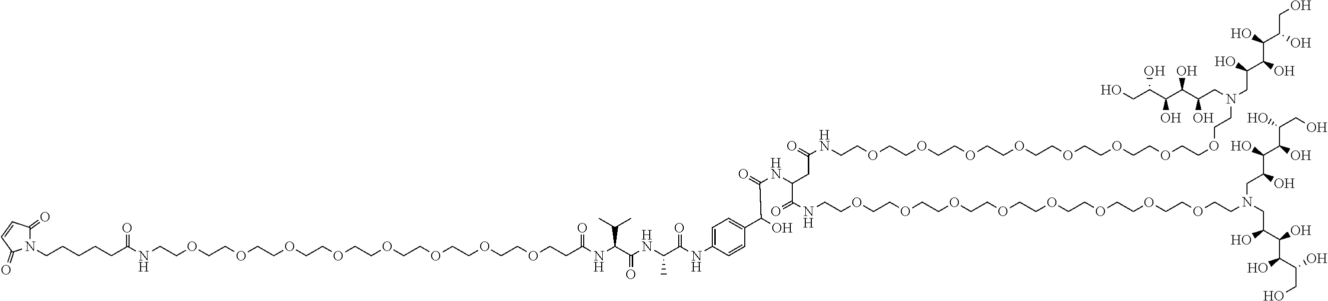 [SMILES: CC(C)[C@H](NC(=O)CCOCCOCCOCCOCCOCCOCCOCCOCCNC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

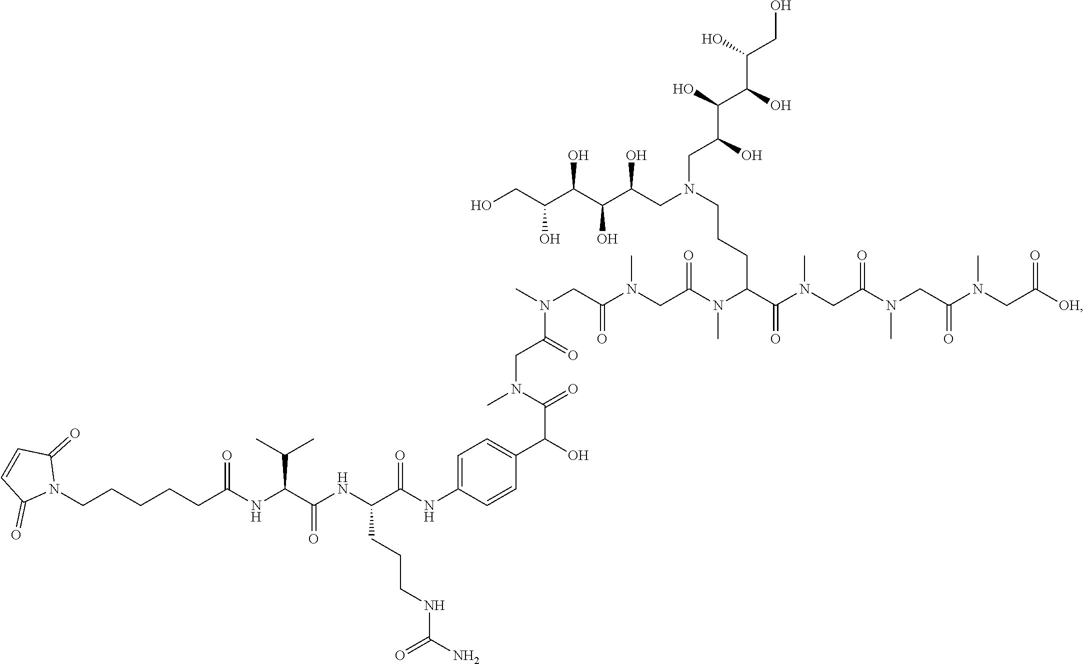 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

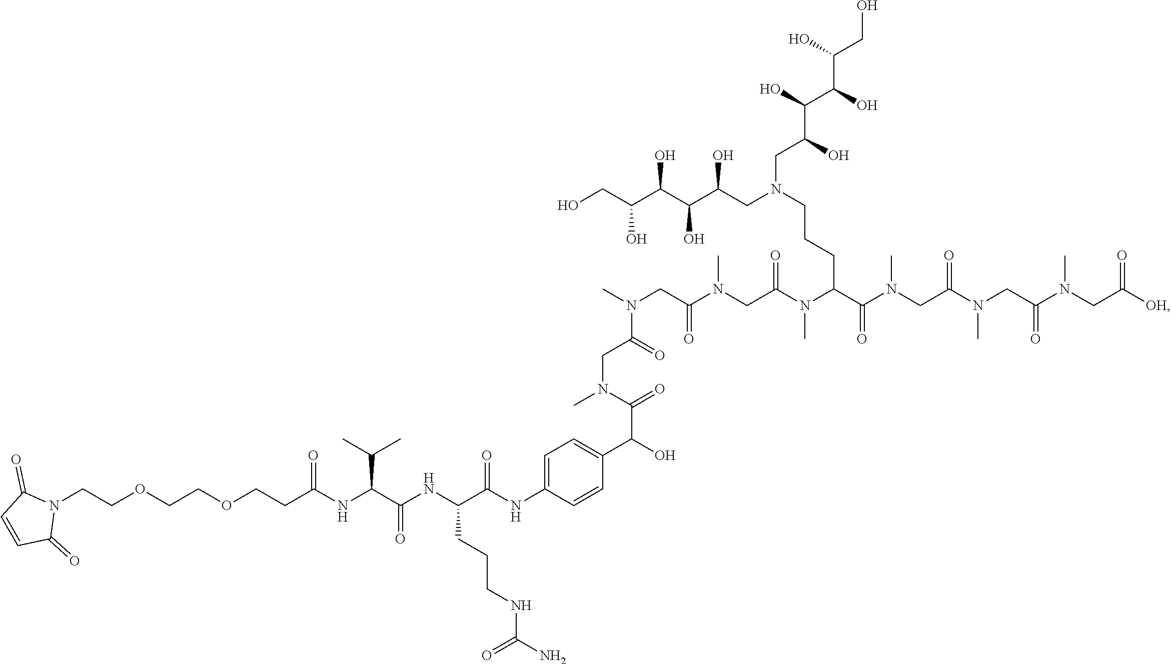 [SMILES: CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

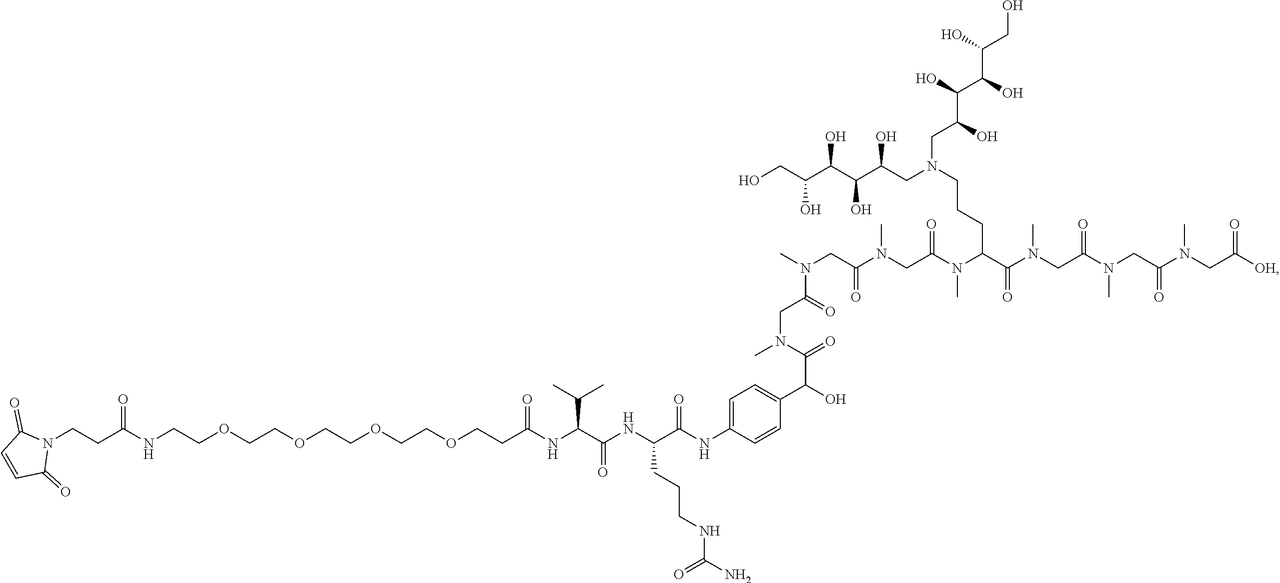 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

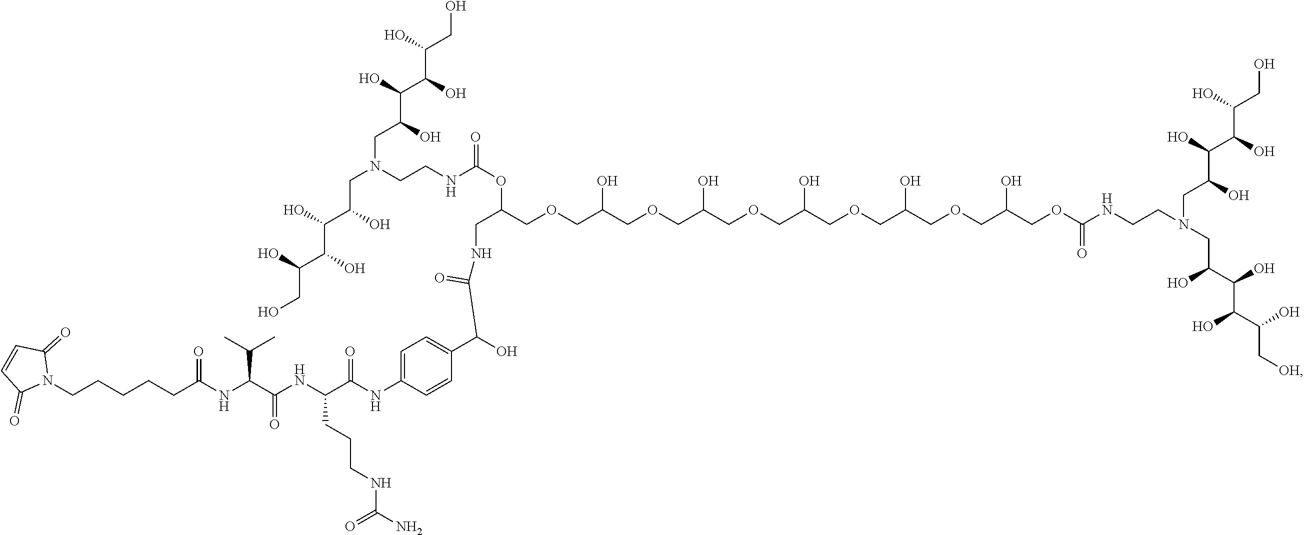 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1]

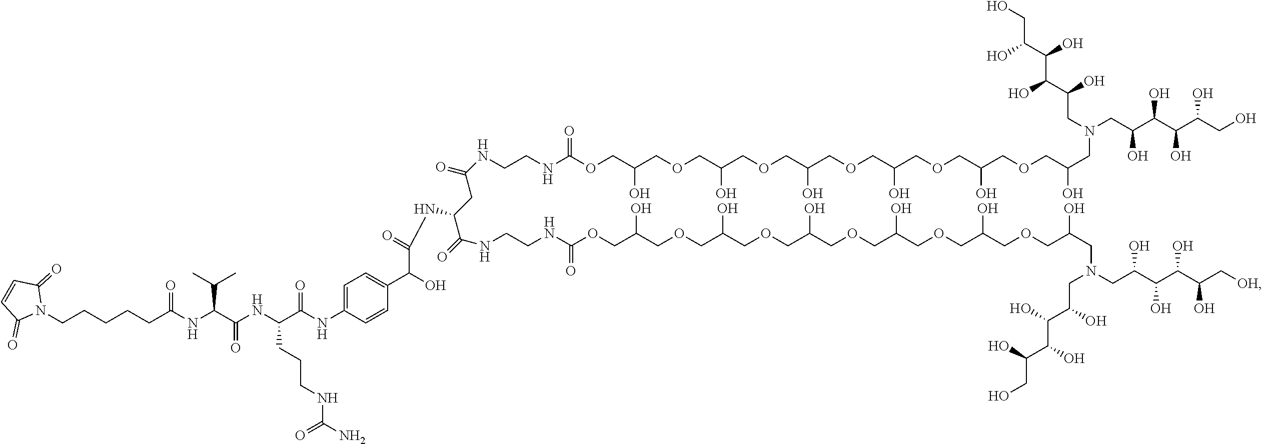 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

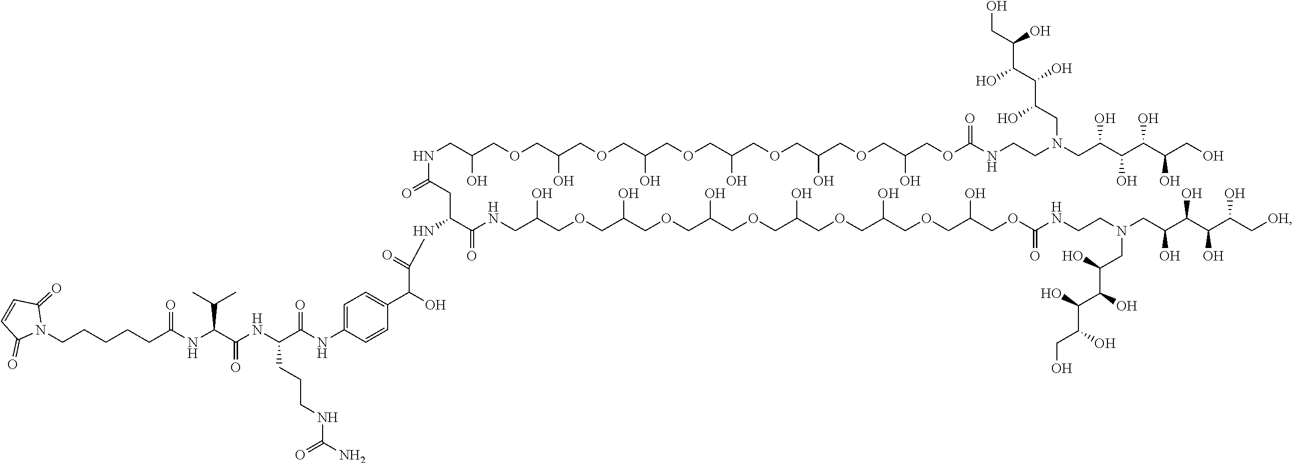 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)cc1]

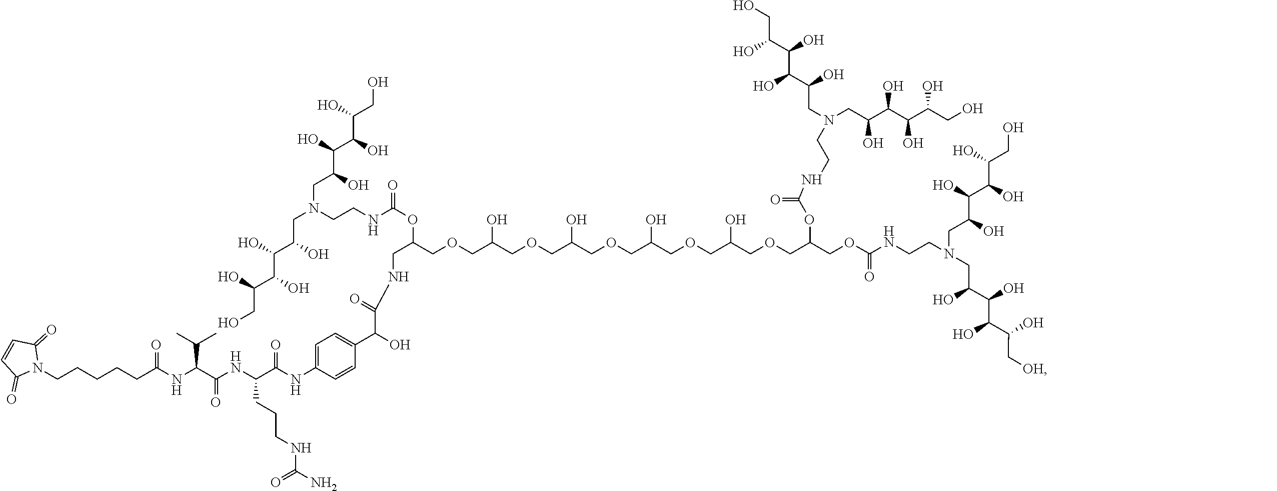 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1]

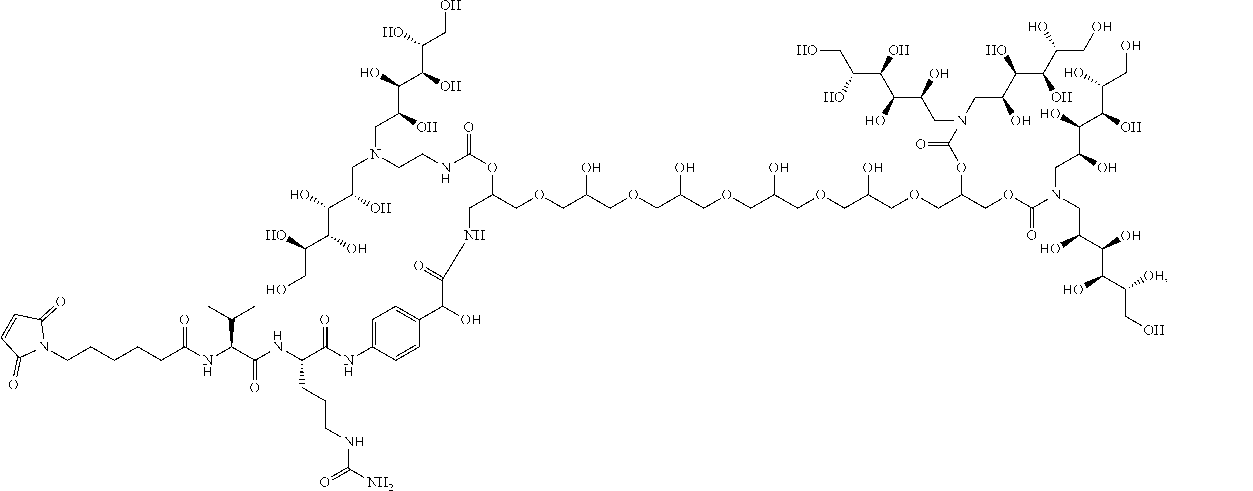 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(COC(=O)N(C[C@H](O)[C@@H](O)[C@H](O)[C@H](O)CO)C[C@H](O)[C@@H](O)[C@H](O)[C@H](O)CO)OC(=O)N(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1]

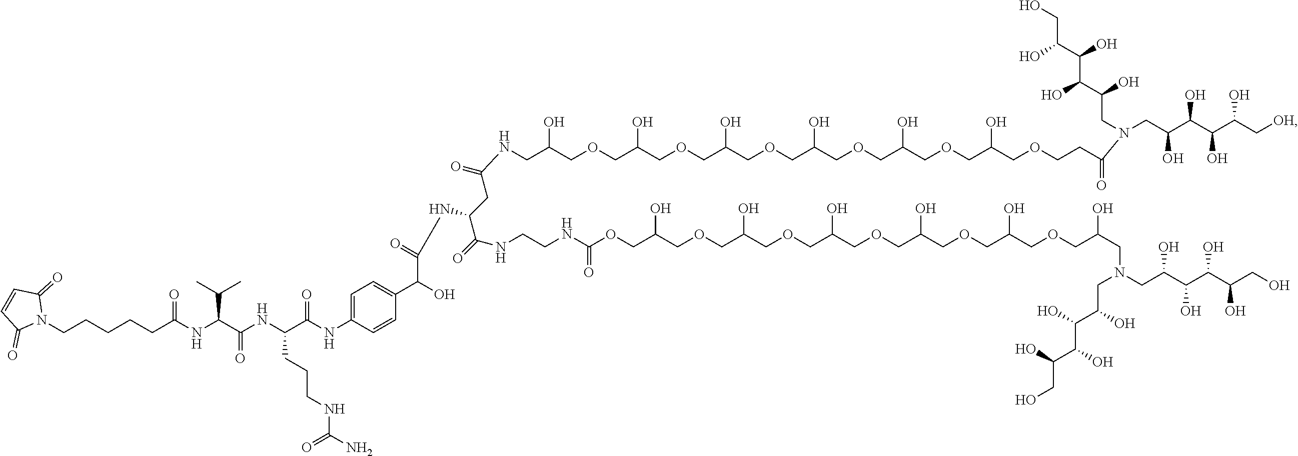 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COCCC(=O)N(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

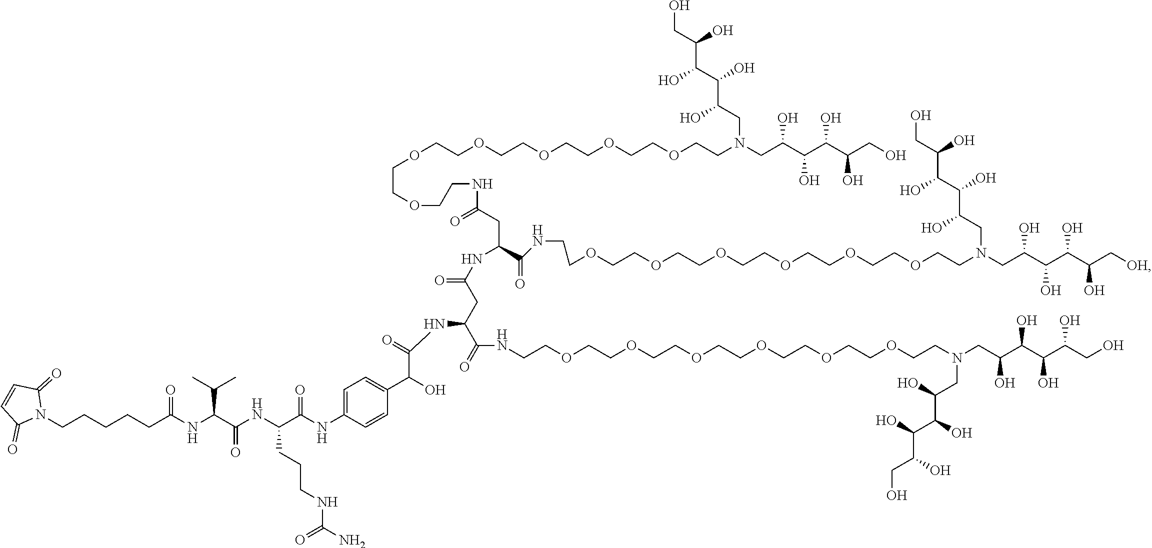 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

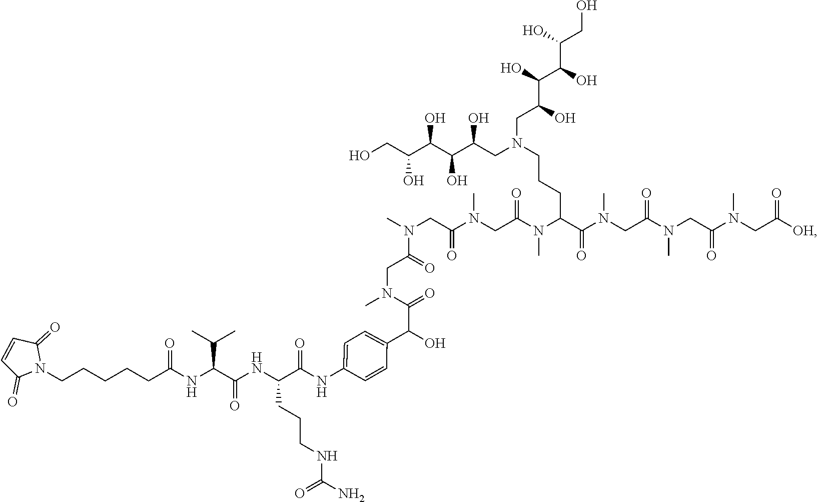 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

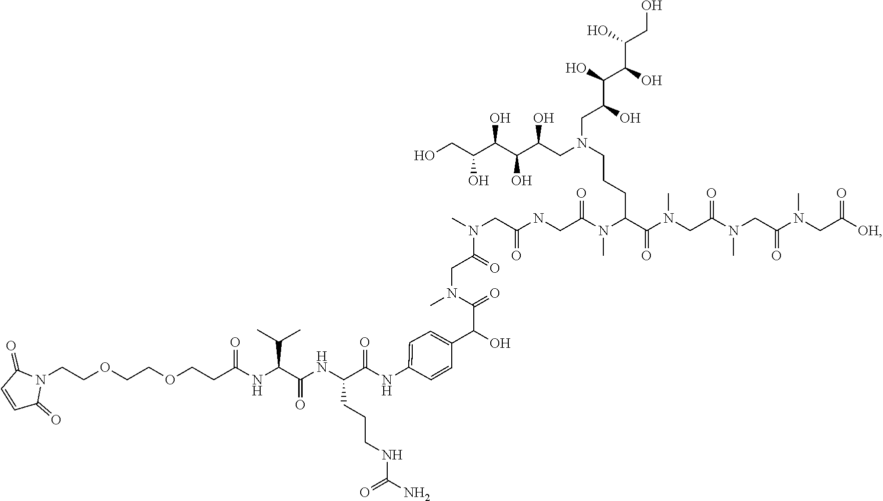 [SMILES: CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)NCC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

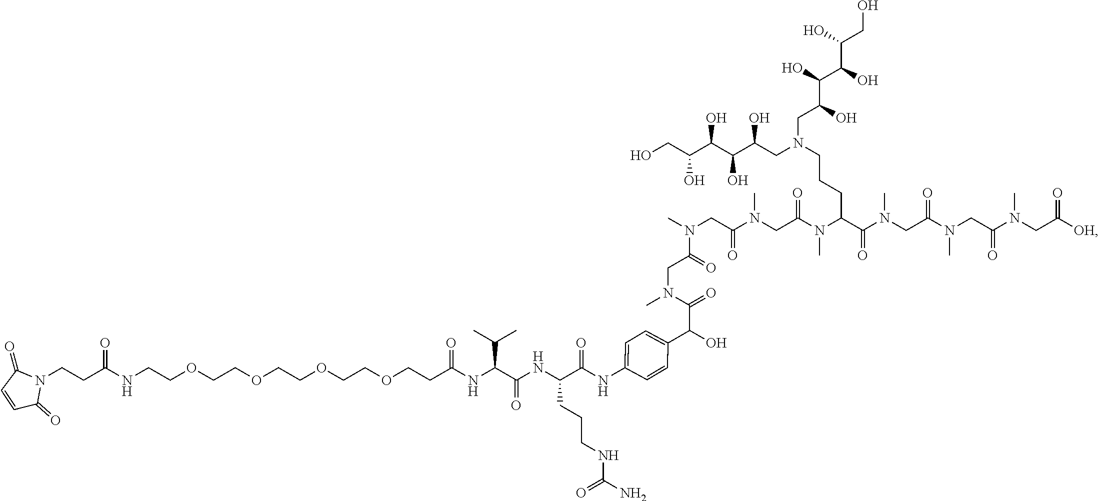 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

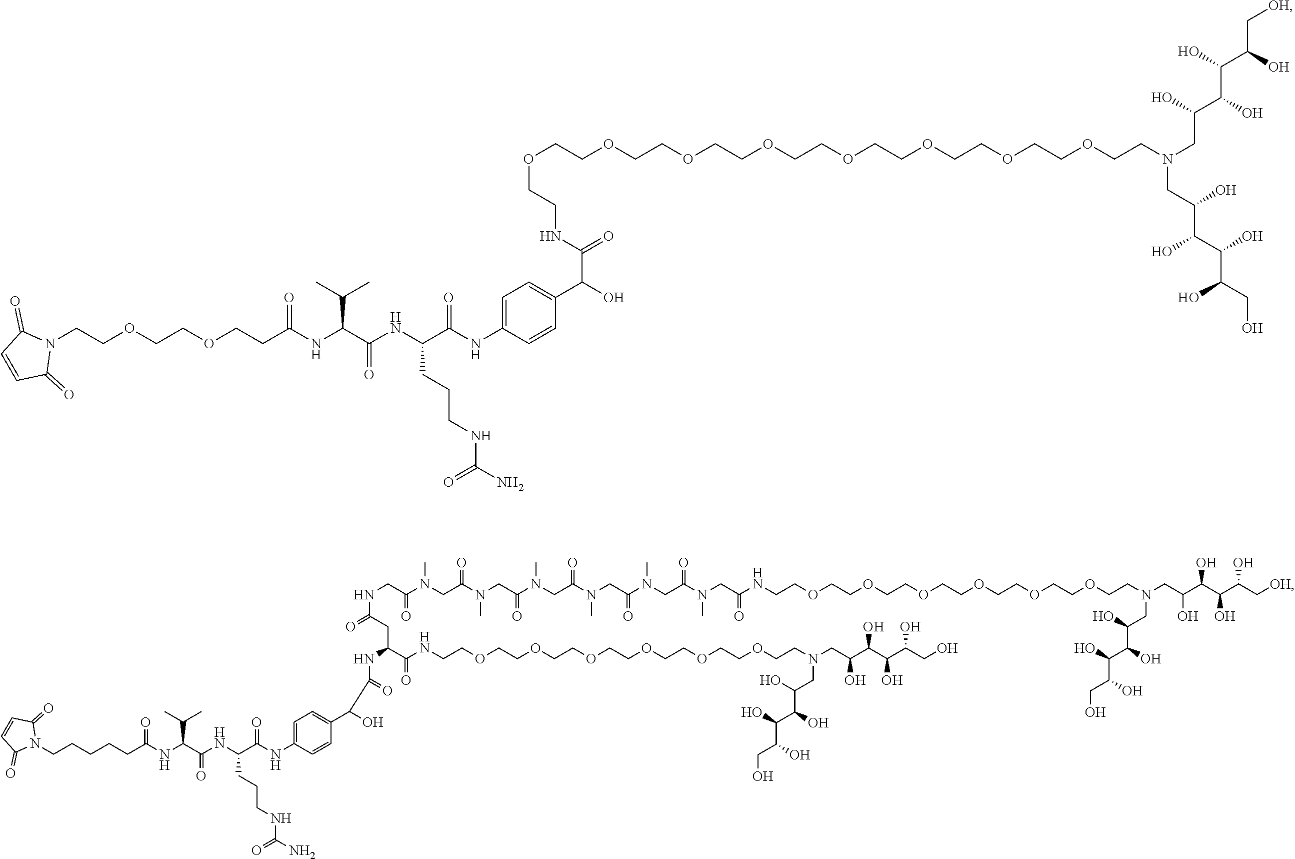

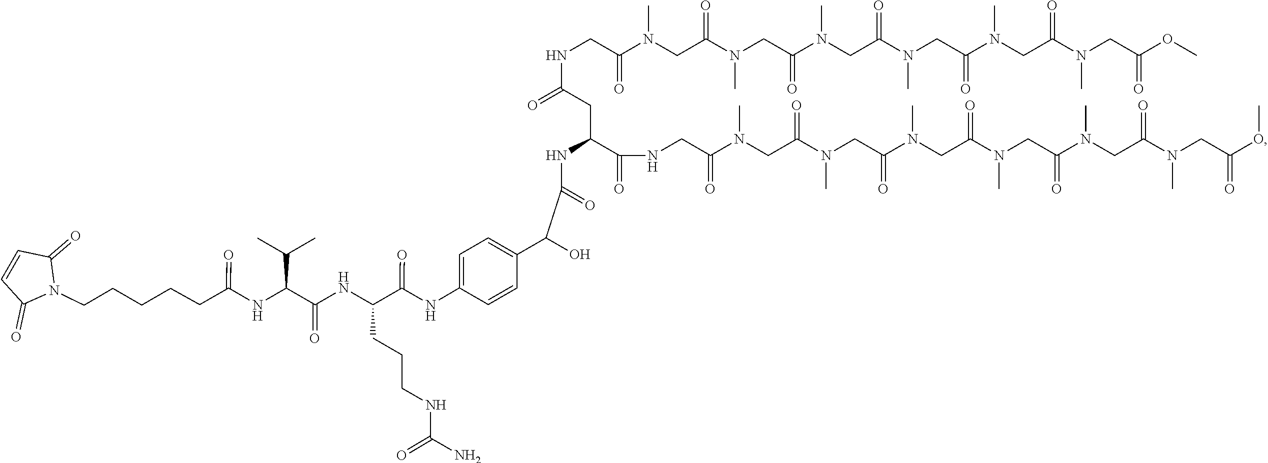 [SMILES: COC(=O)CN(C)C(=O)CN(C)C(=O)CN(C)C(=O)CN(C)C(=O)CN(C)C(=O)CN(C)C(=O)CNC(=O)C[C@H](NC(=O)C(O)c1ccc(NC(=O)[C@H](CCCNC(N)=O)NC(=O)[C@@H](NC(=O)CCCCCN2C(=O)C=CC2=O)C(C)C)cc1)C(=O)NCC(=O)N(C)CC(=O)N(C)CC(=O)N(C)CC(=O)N(C)CC(=O)N(C)CC(=O)N(C)CC(=O)OC]

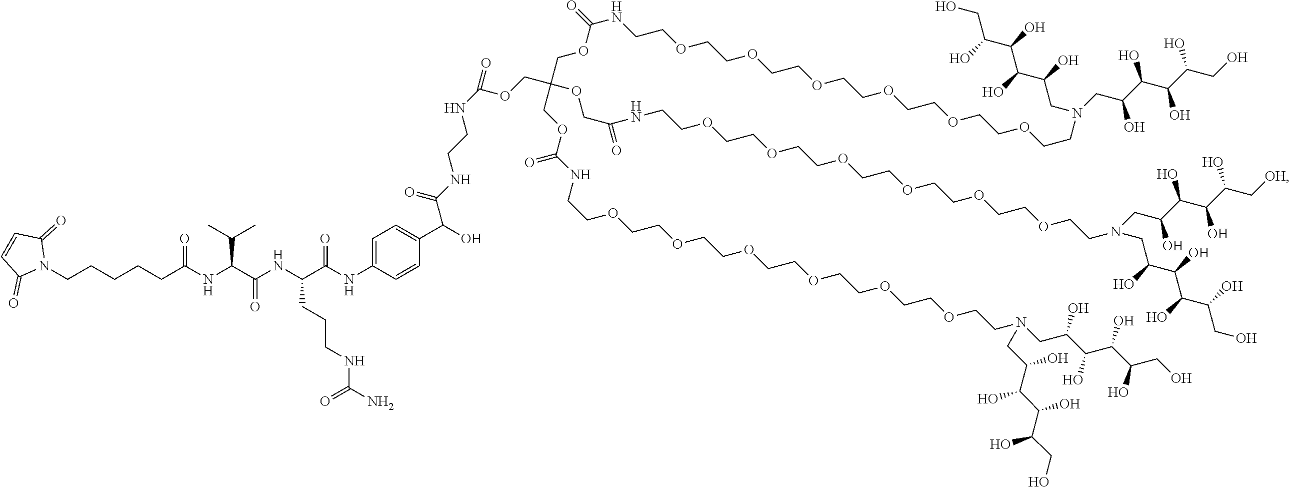 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCNC(=O)OCC(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)OCC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

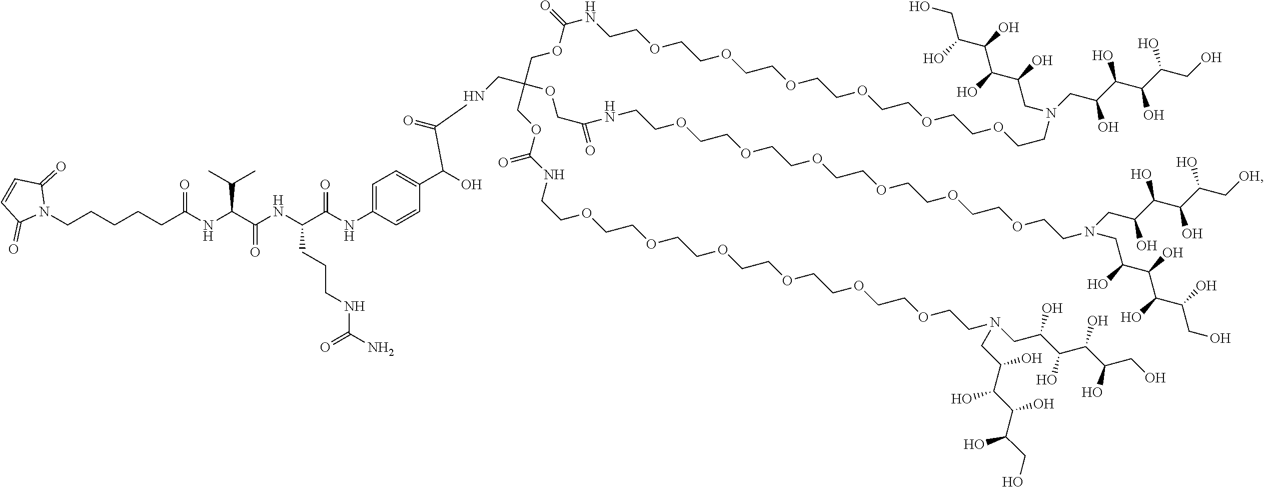 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)OCC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

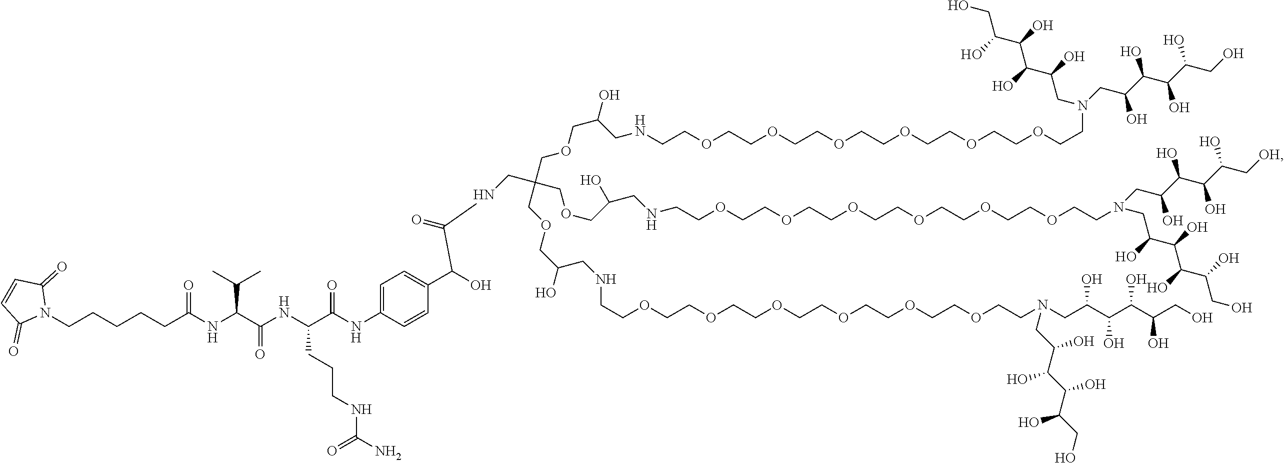 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)CNCCOCCOCCOCCOCCOCCOCCN(C[C@H](O)[C@@H](O)[C@H](O)[C@H](O)CO)C[C@H](O)[C@@H](O)[C@H](O)[C@H](O)CO)(COCC(O)CNCCOCCOCCOCCOCCOCCOCCN(C[C@H](O)[C@@H](O)[C@H](O)[C@H](O)CO)C[C@H](O)[C@@H](O)[C@H](O)[C@H](O)CO)COCC(O)CNCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

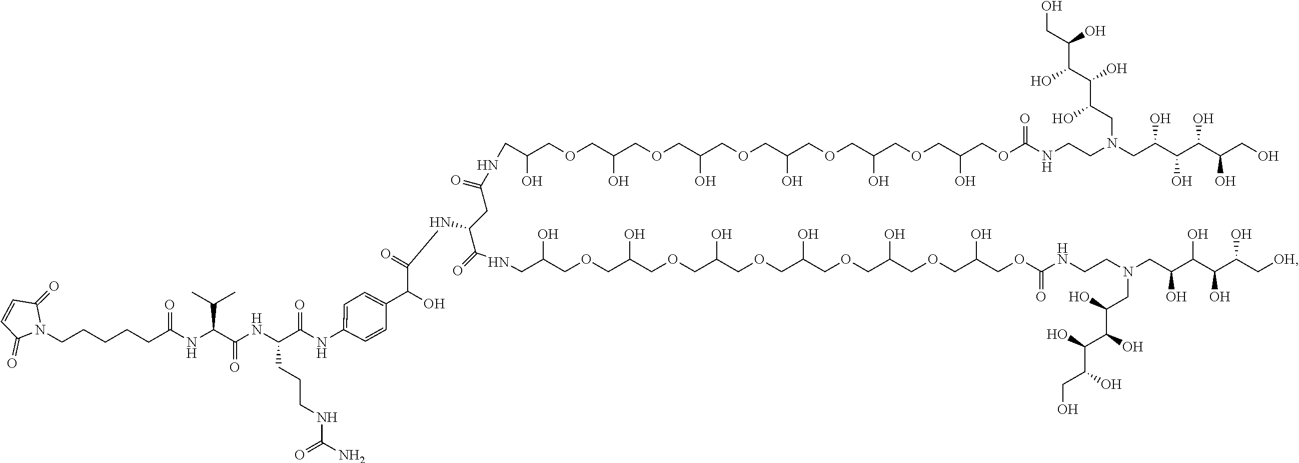 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)C(O)[C@H](O)[C@H](O)CO)C[C@H](O)[C@@H](O)[C@H](O)[C@H](O)CO)cc1]

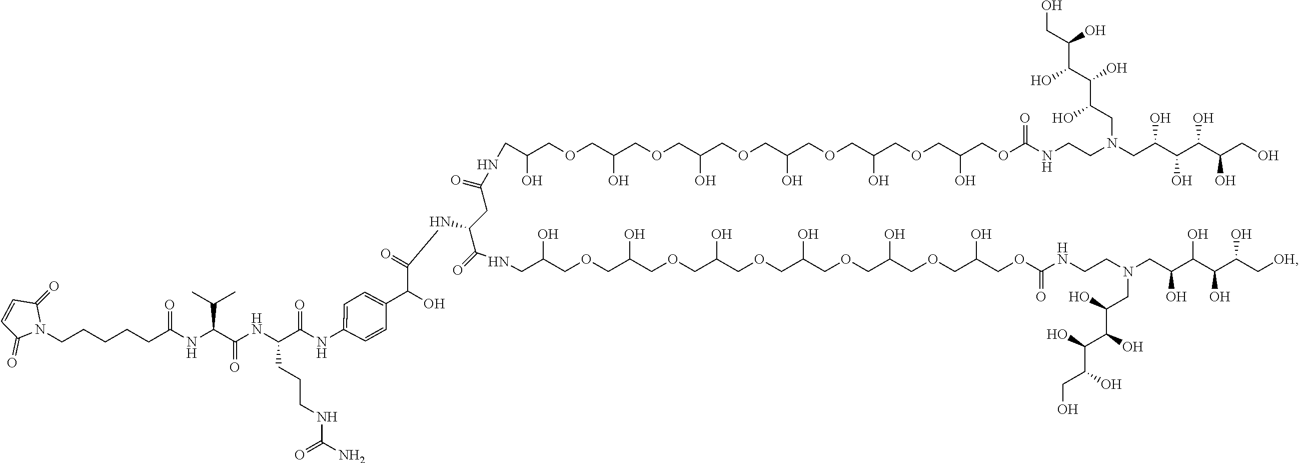 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)C(O)[C@H](O)[C@H](O)CO)C[C@H](O)[C@@H](O)[C@H](O)[C@H](O)CO)cc1]

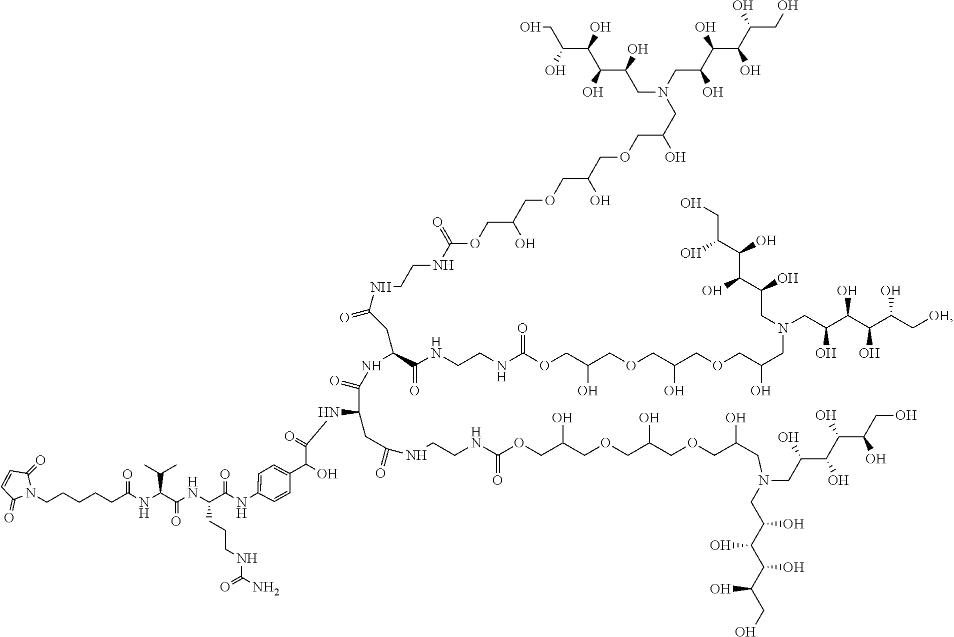 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

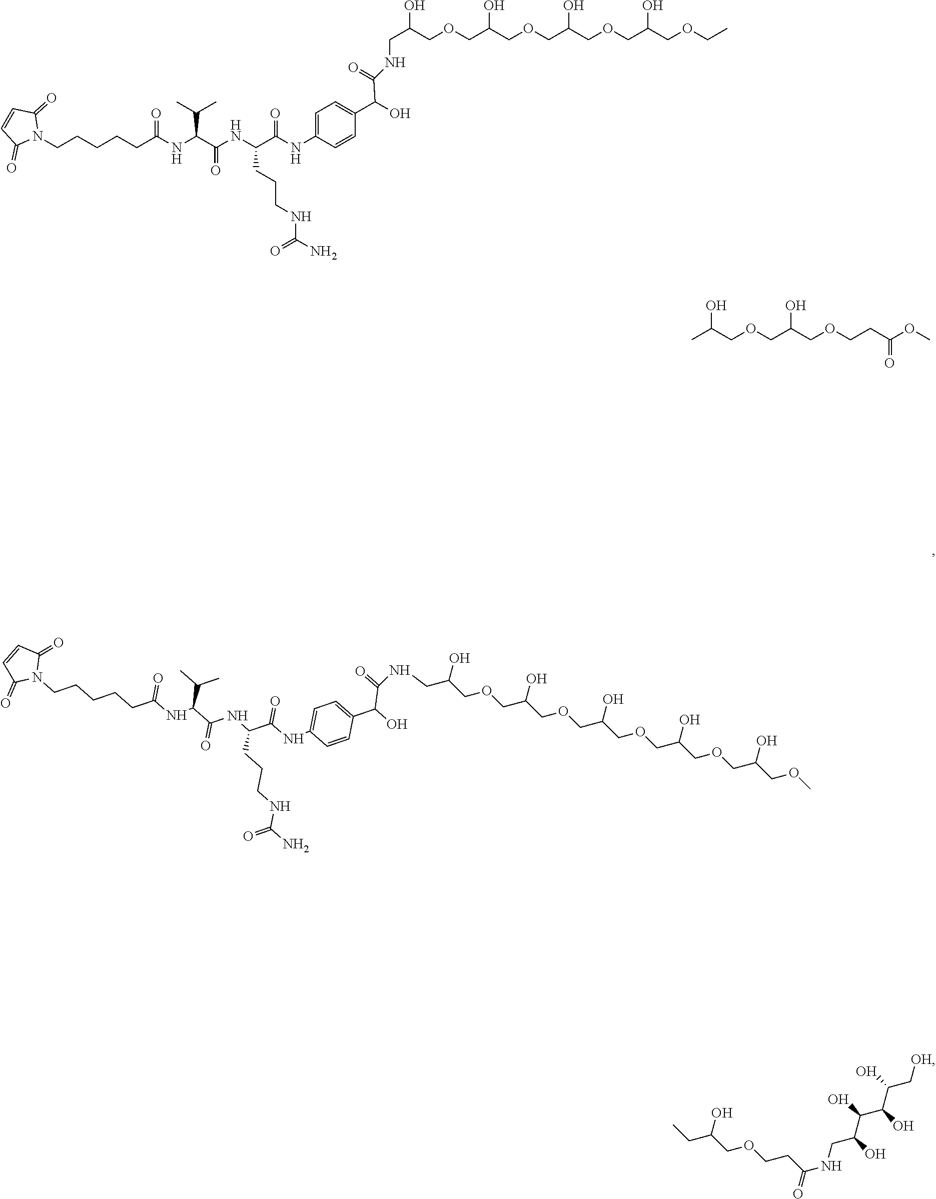

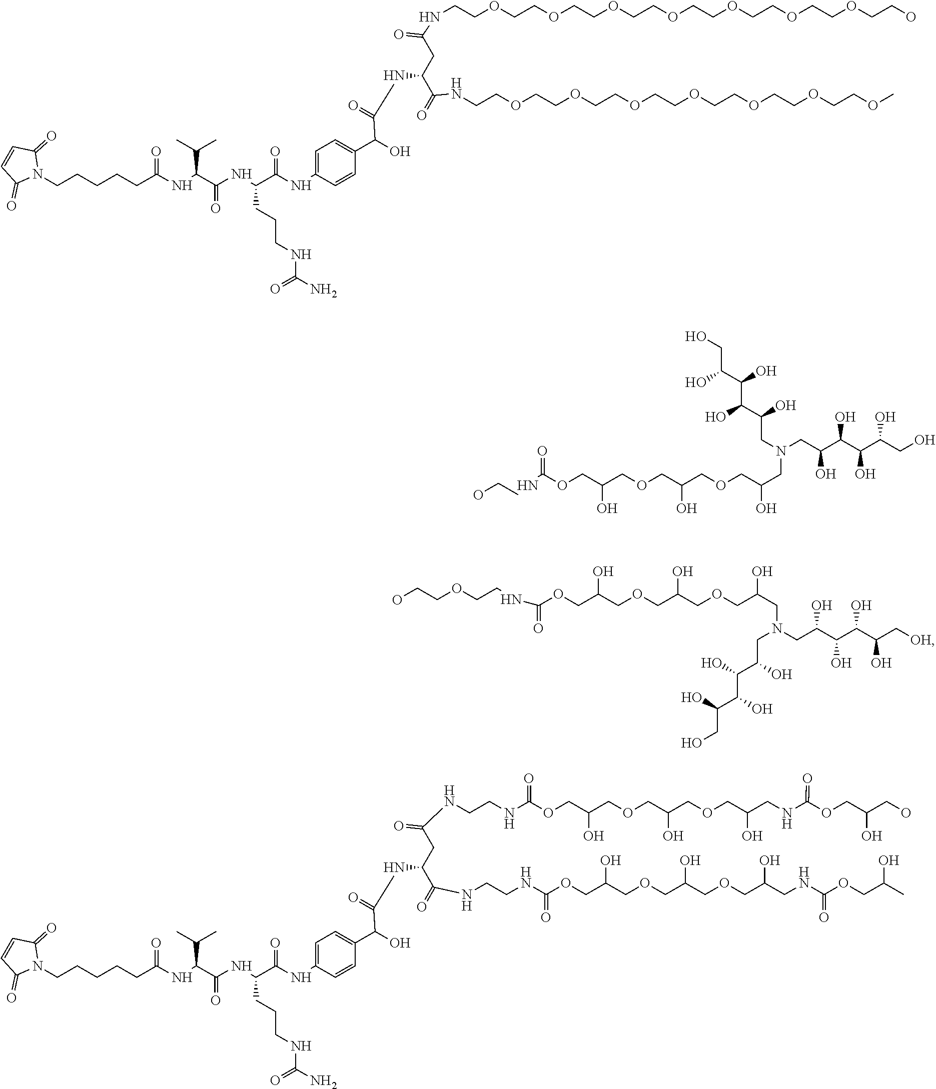

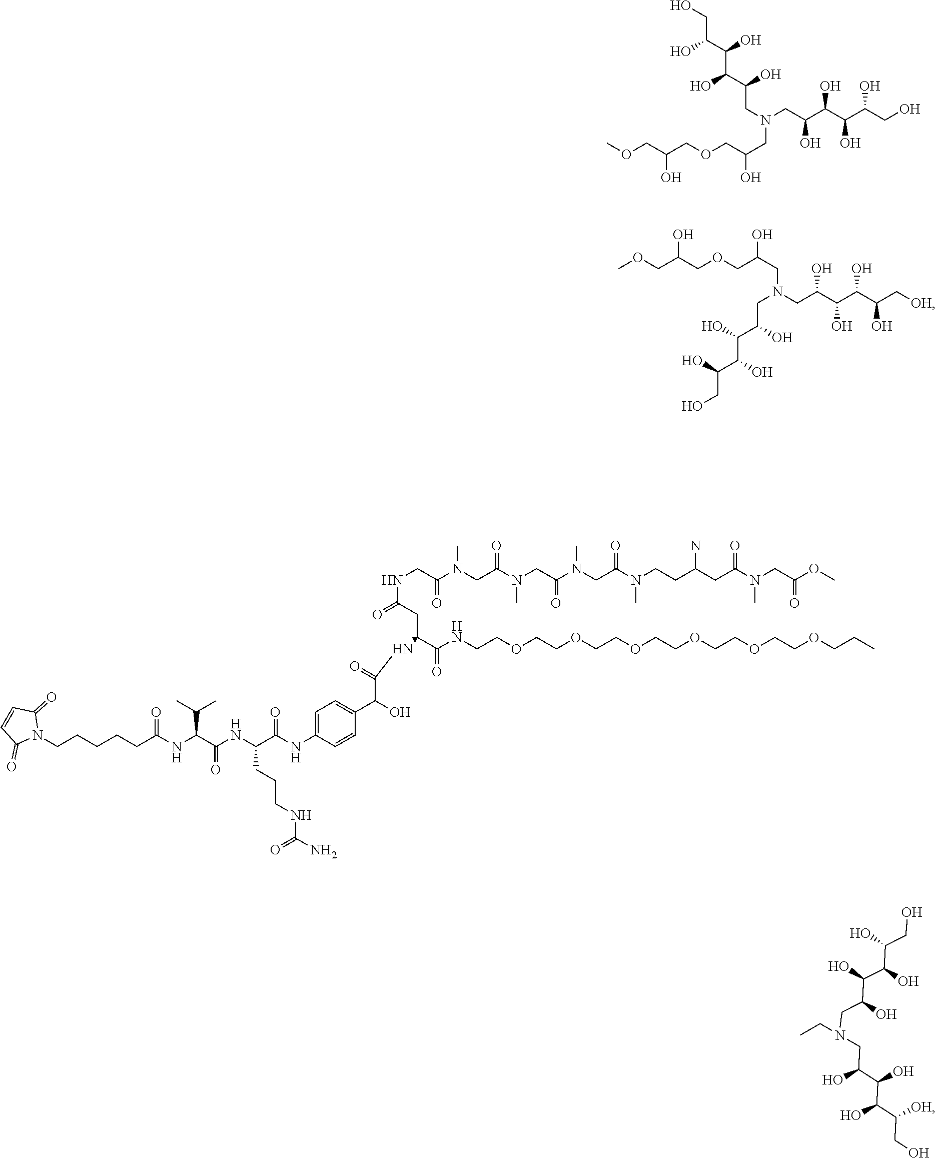

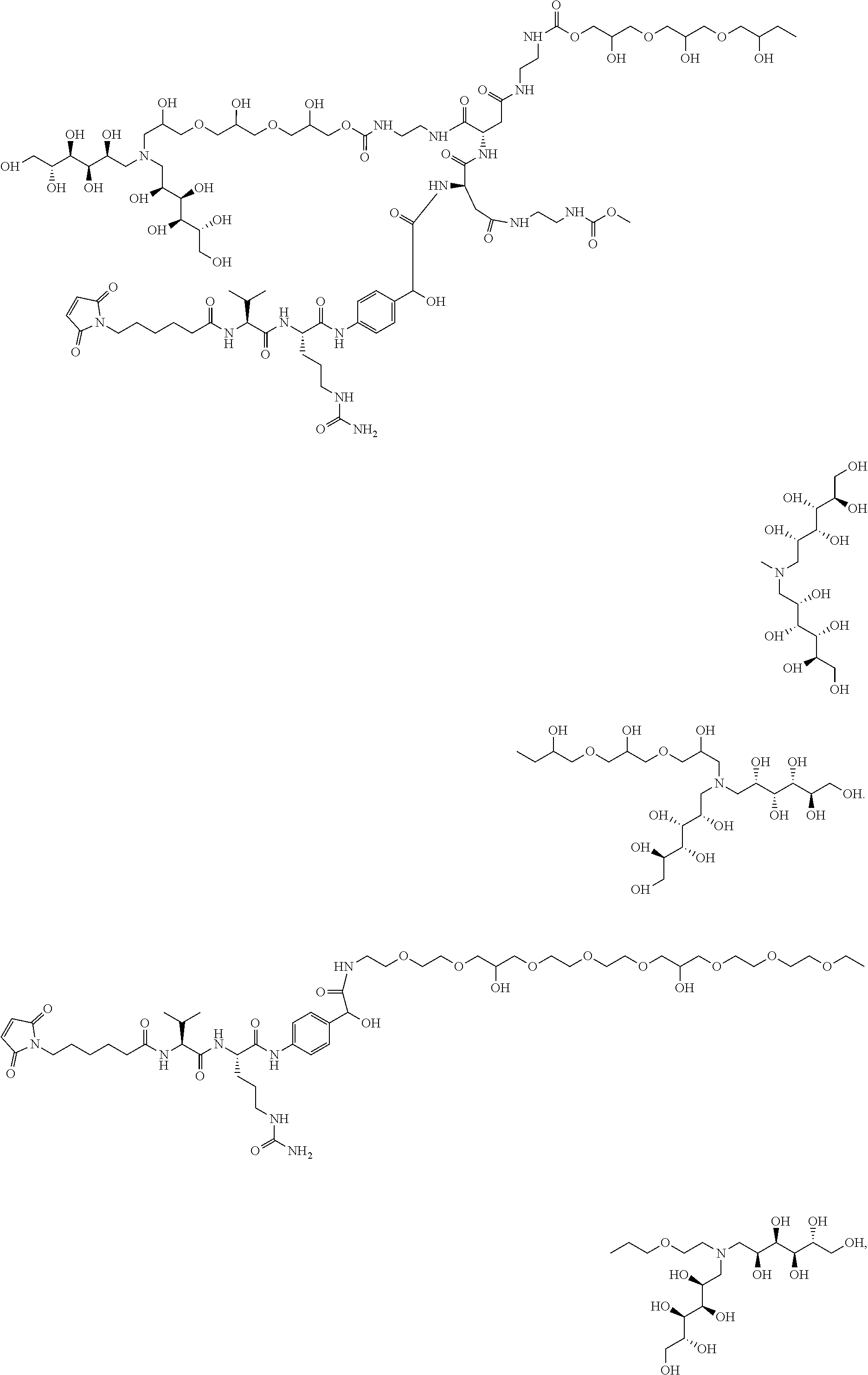

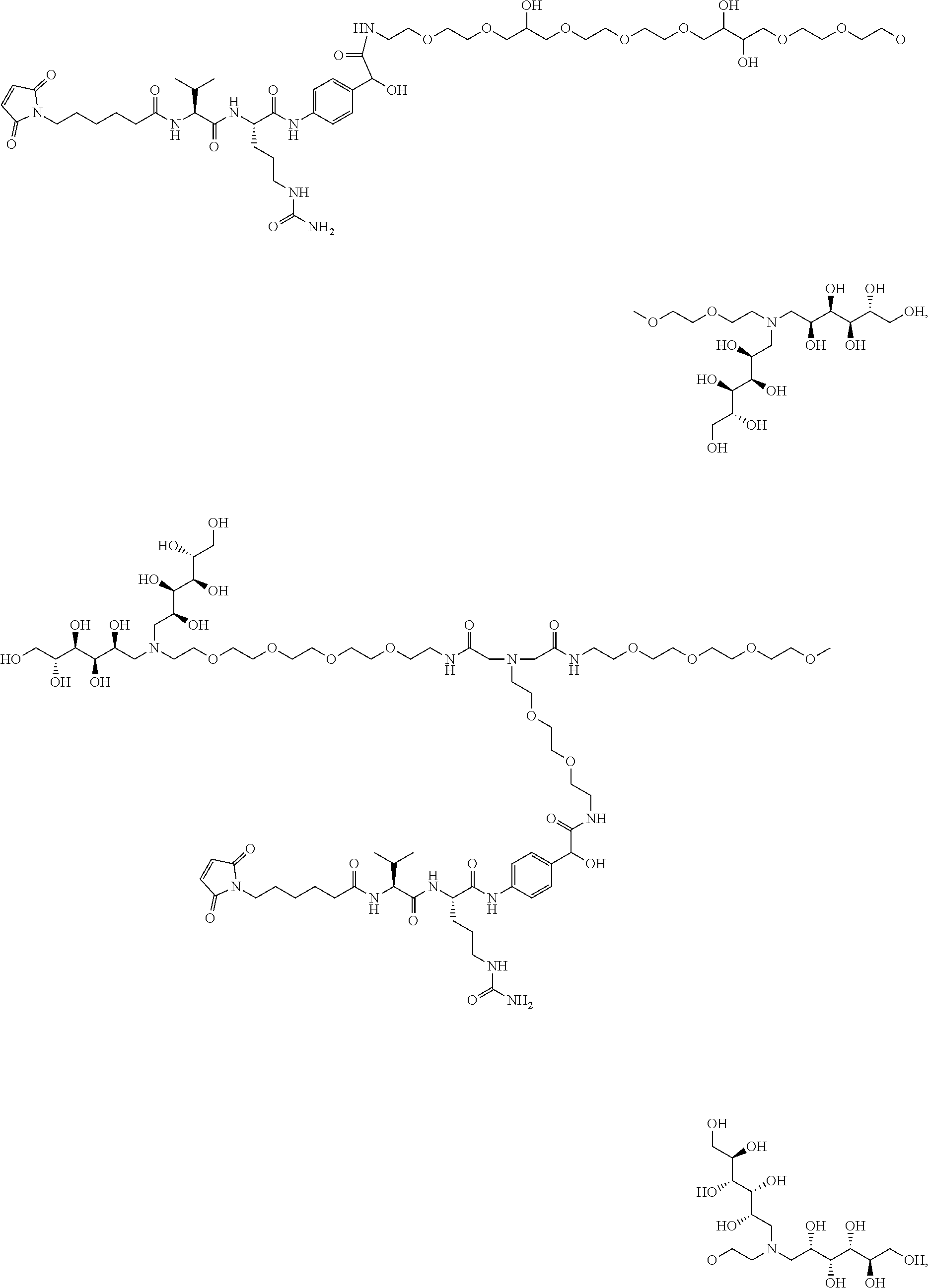

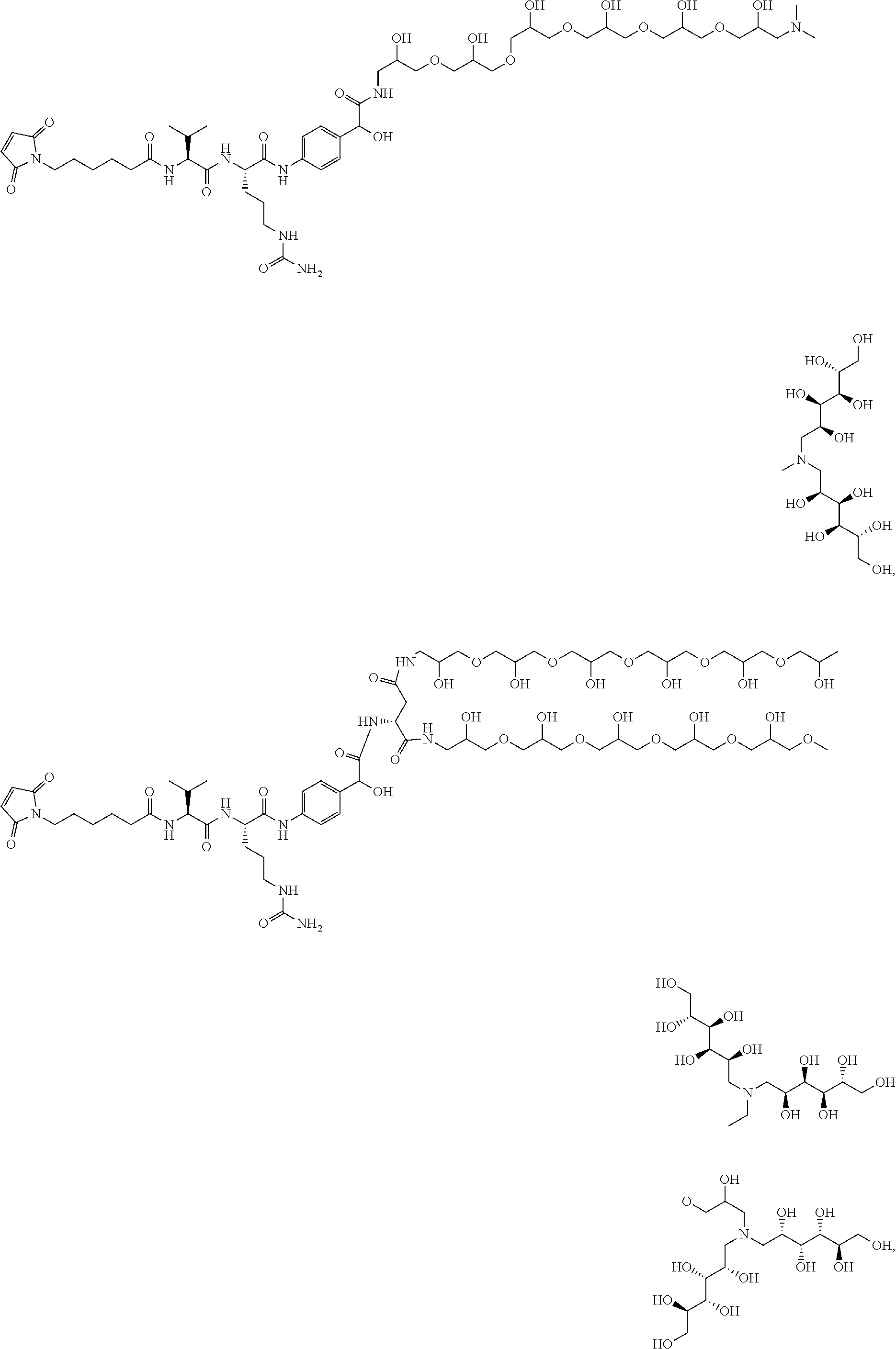

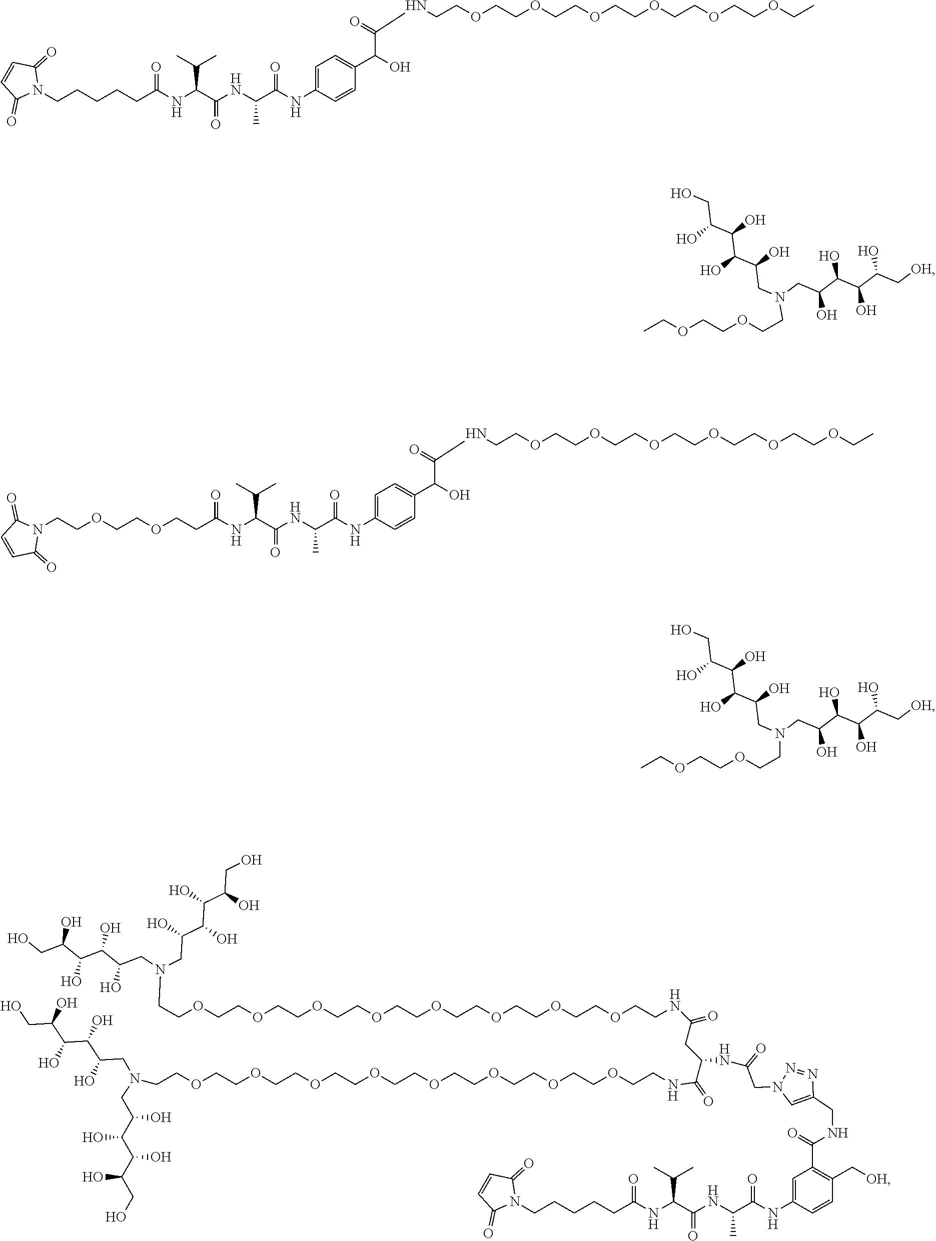

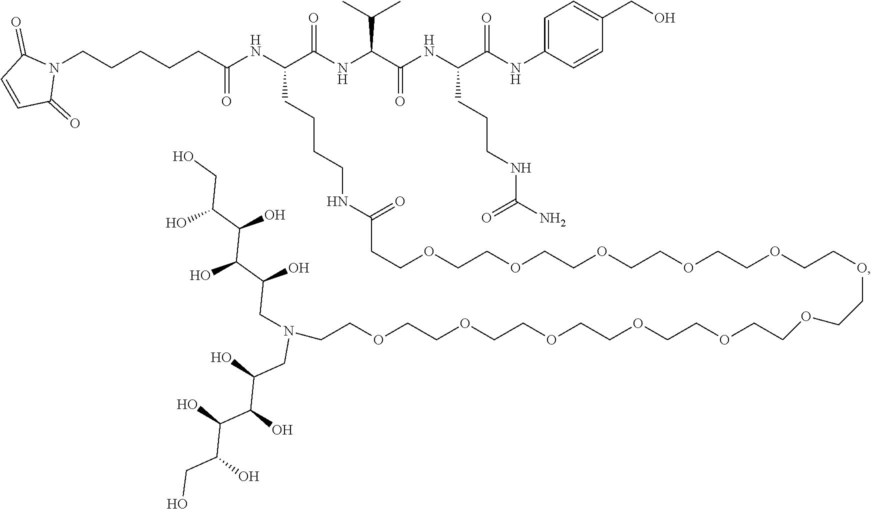 [SMILES: CC(C)[C@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN1C(=O)C=CC1=O)C(=O)N[C@@H](CCCNC(N)=O)C(=O)Nc1ccc(CO)cc1]

wherein the H on the benzylic OH is optionally replaced with a bond to the drug unit or to a linking group attached to the drug unit.

Embodiment 27. The linker compound of any one of Embodiments 1-25, having one of the following structures:

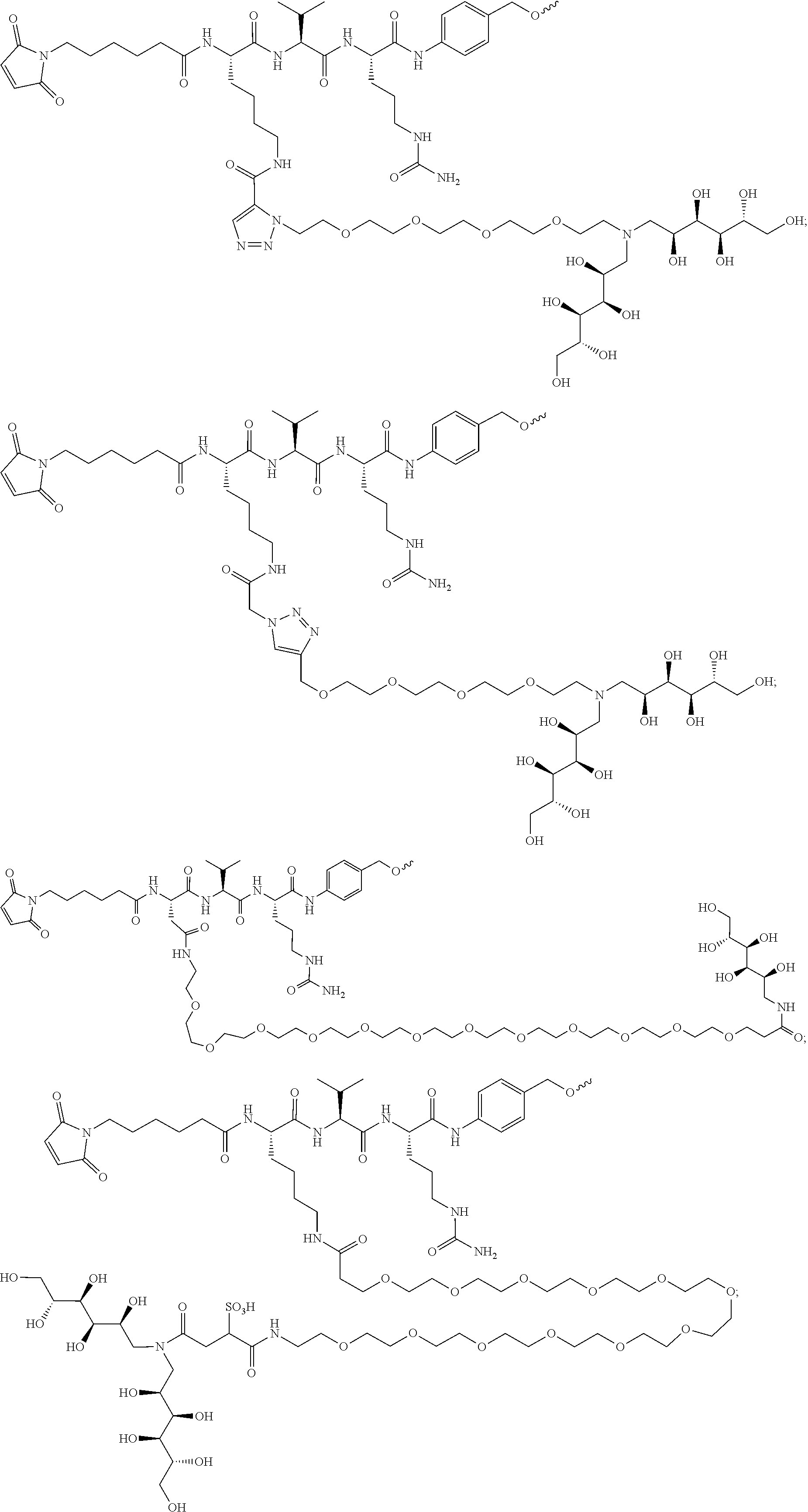

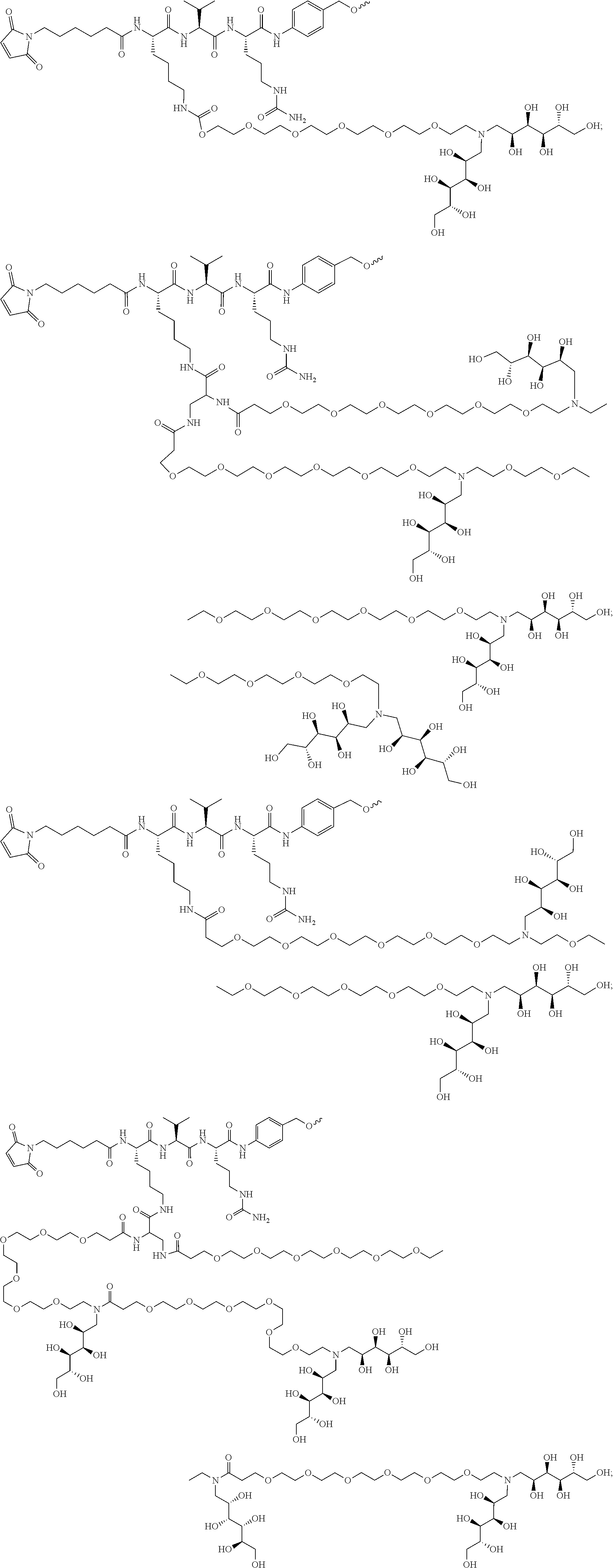

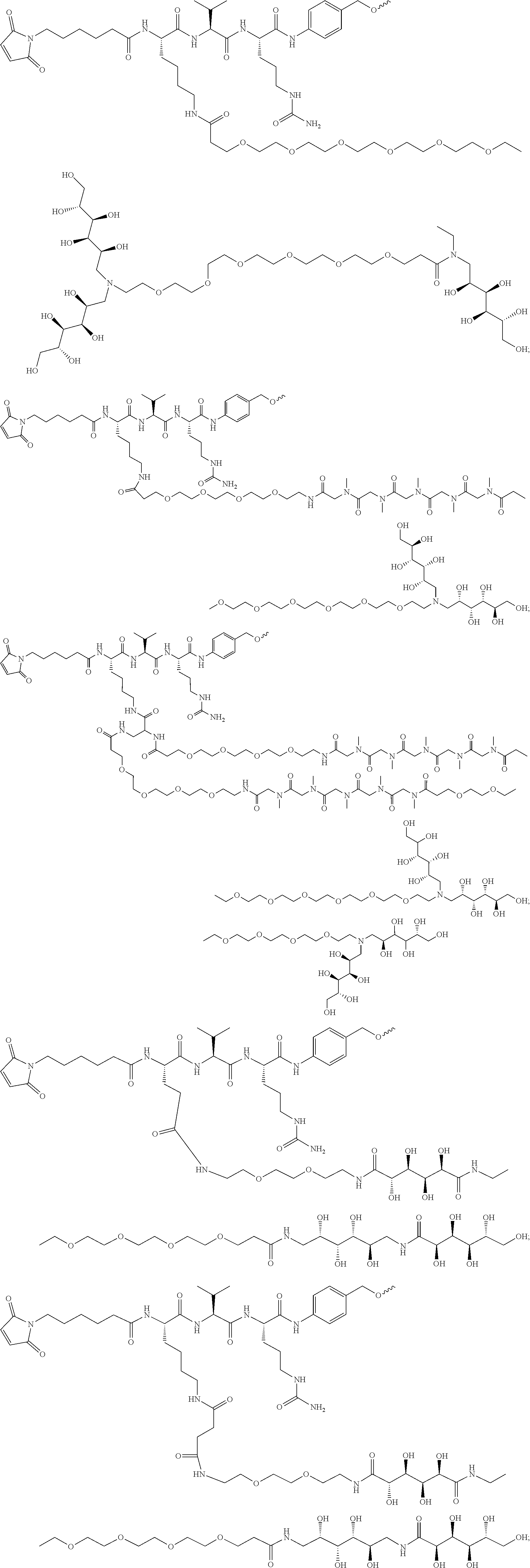

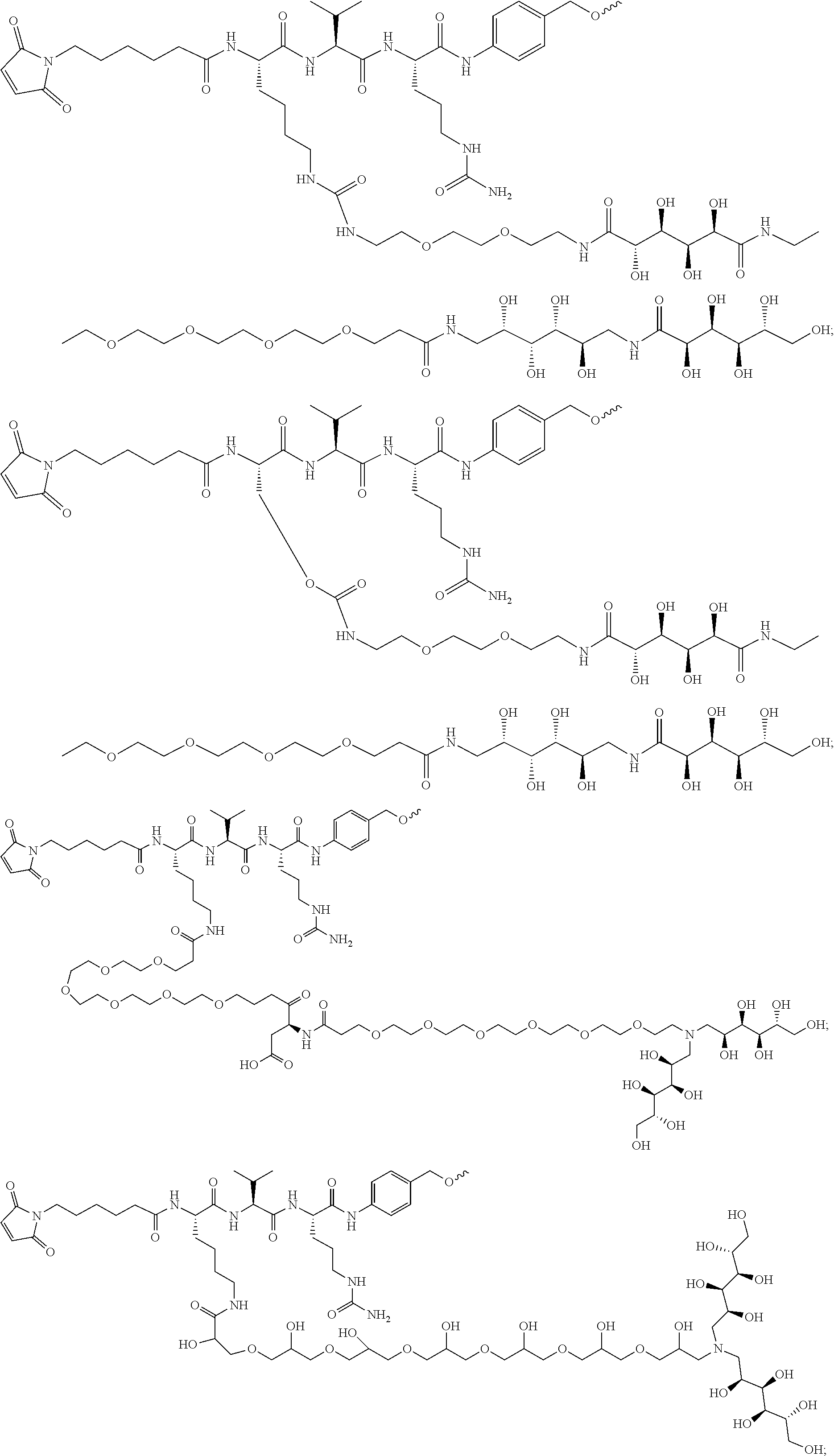

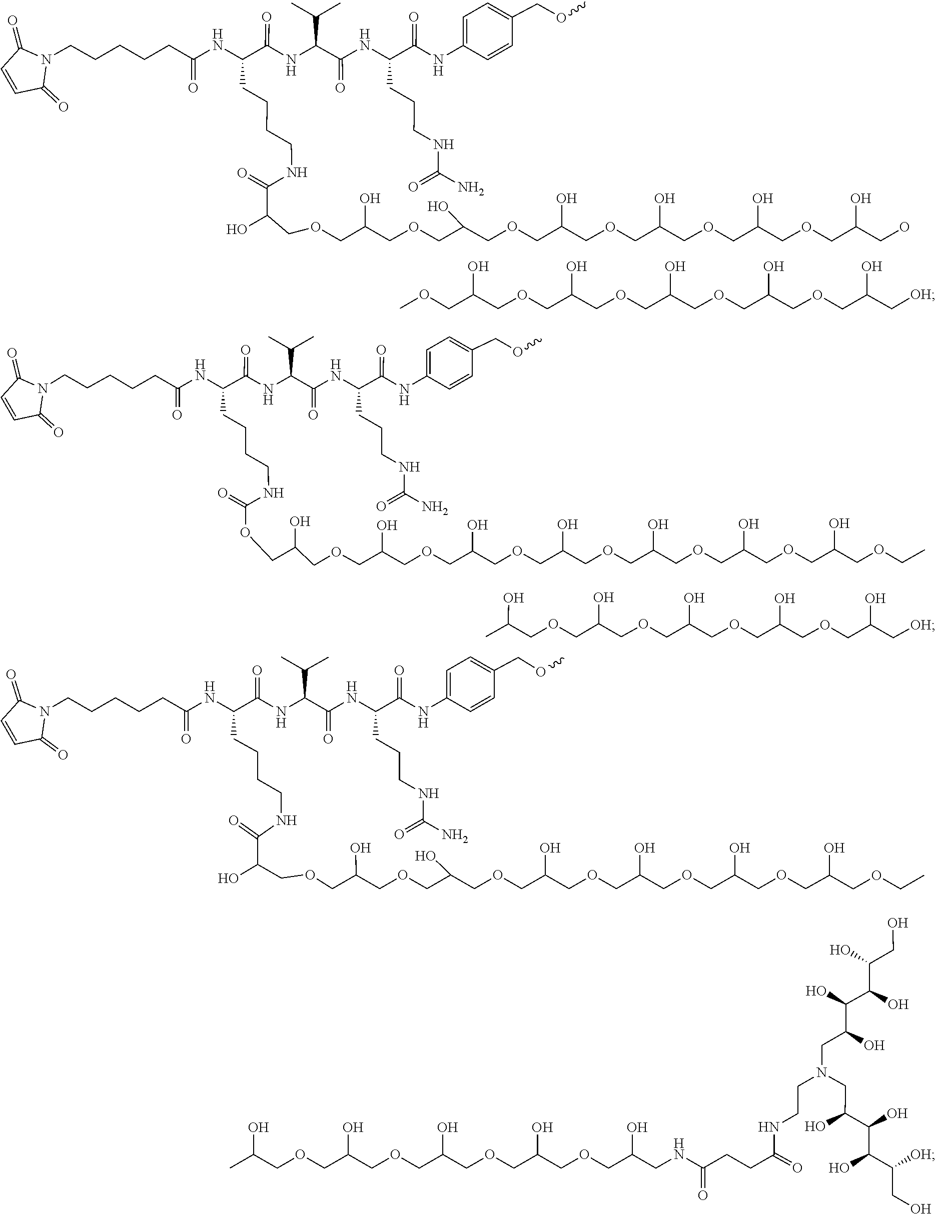

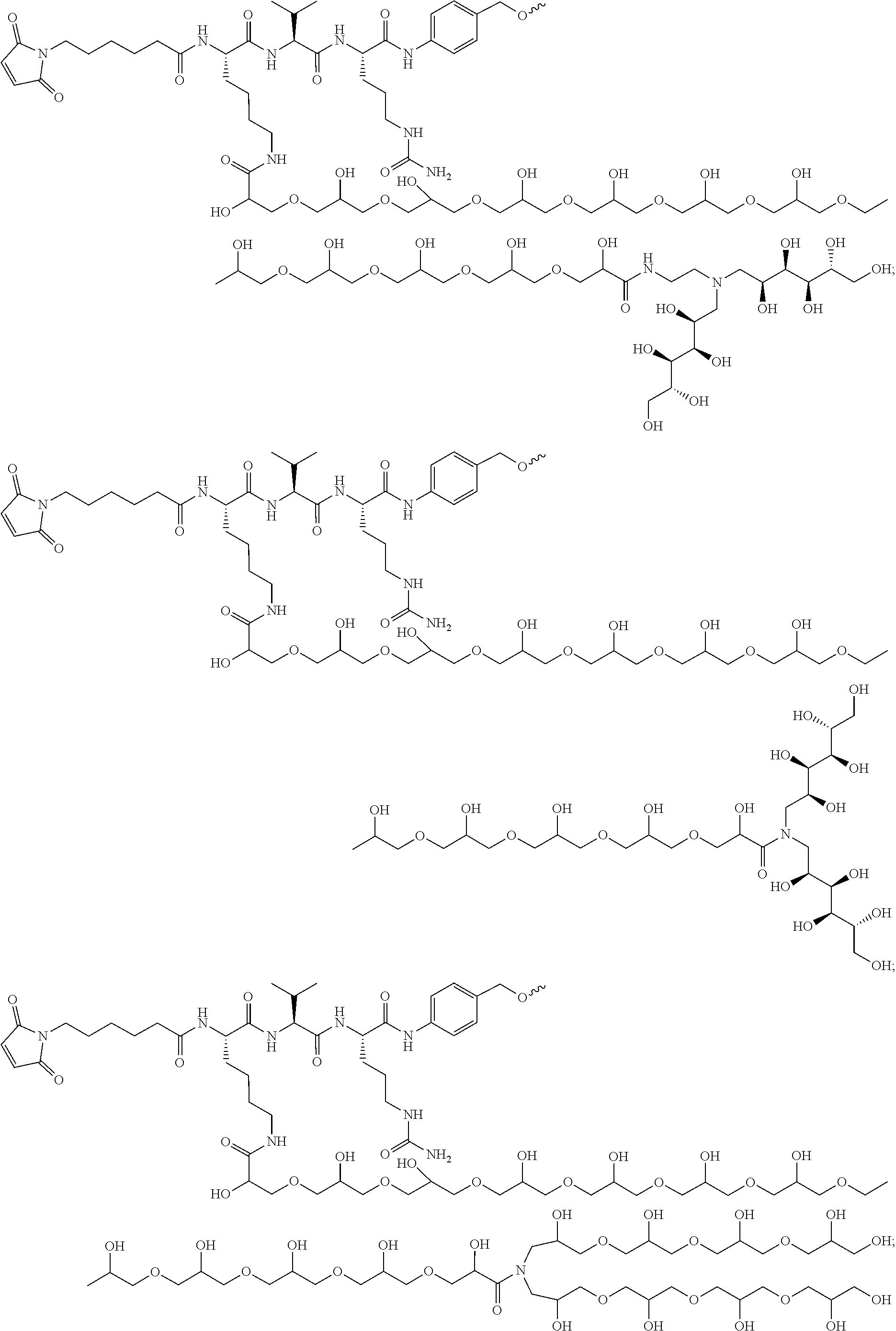

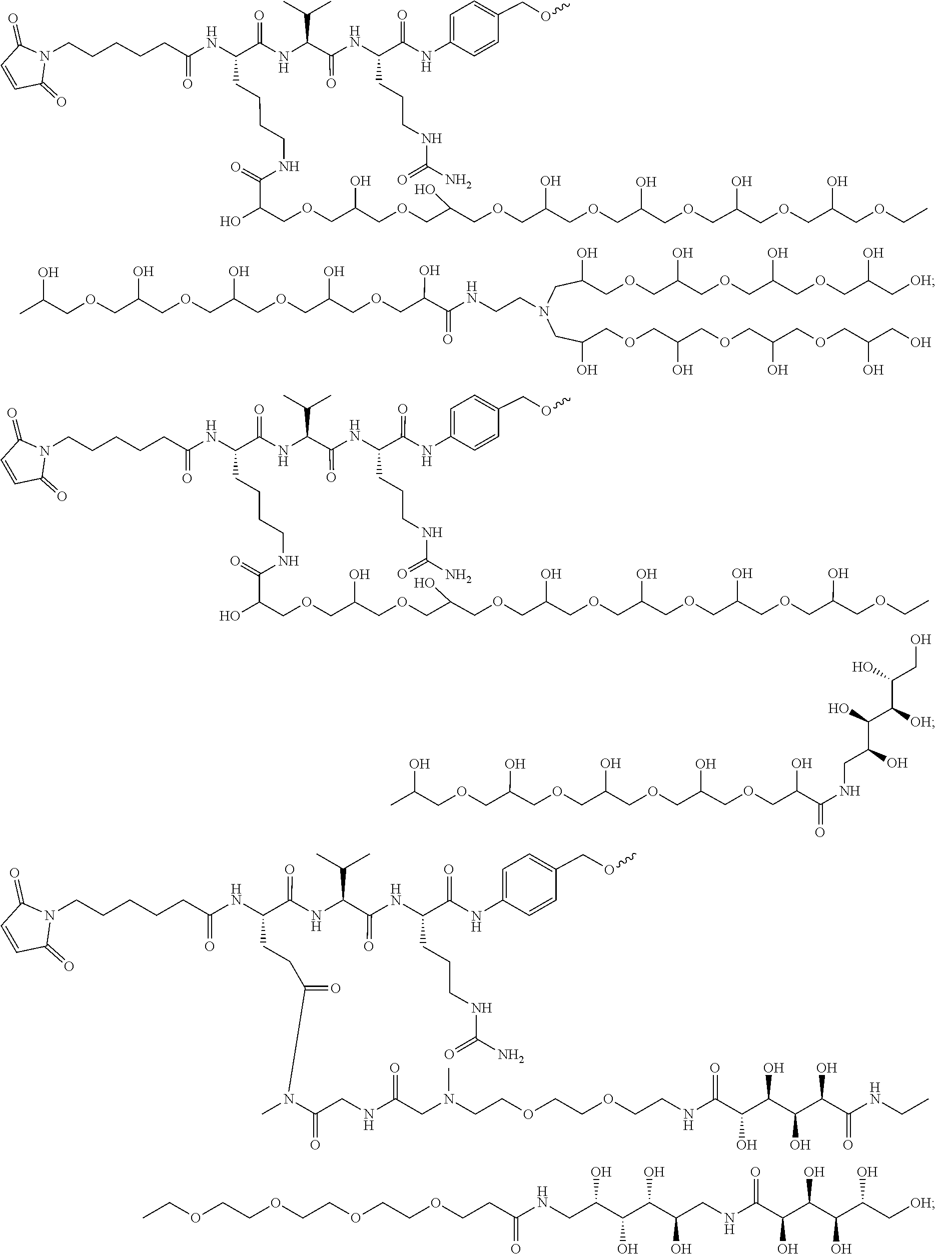

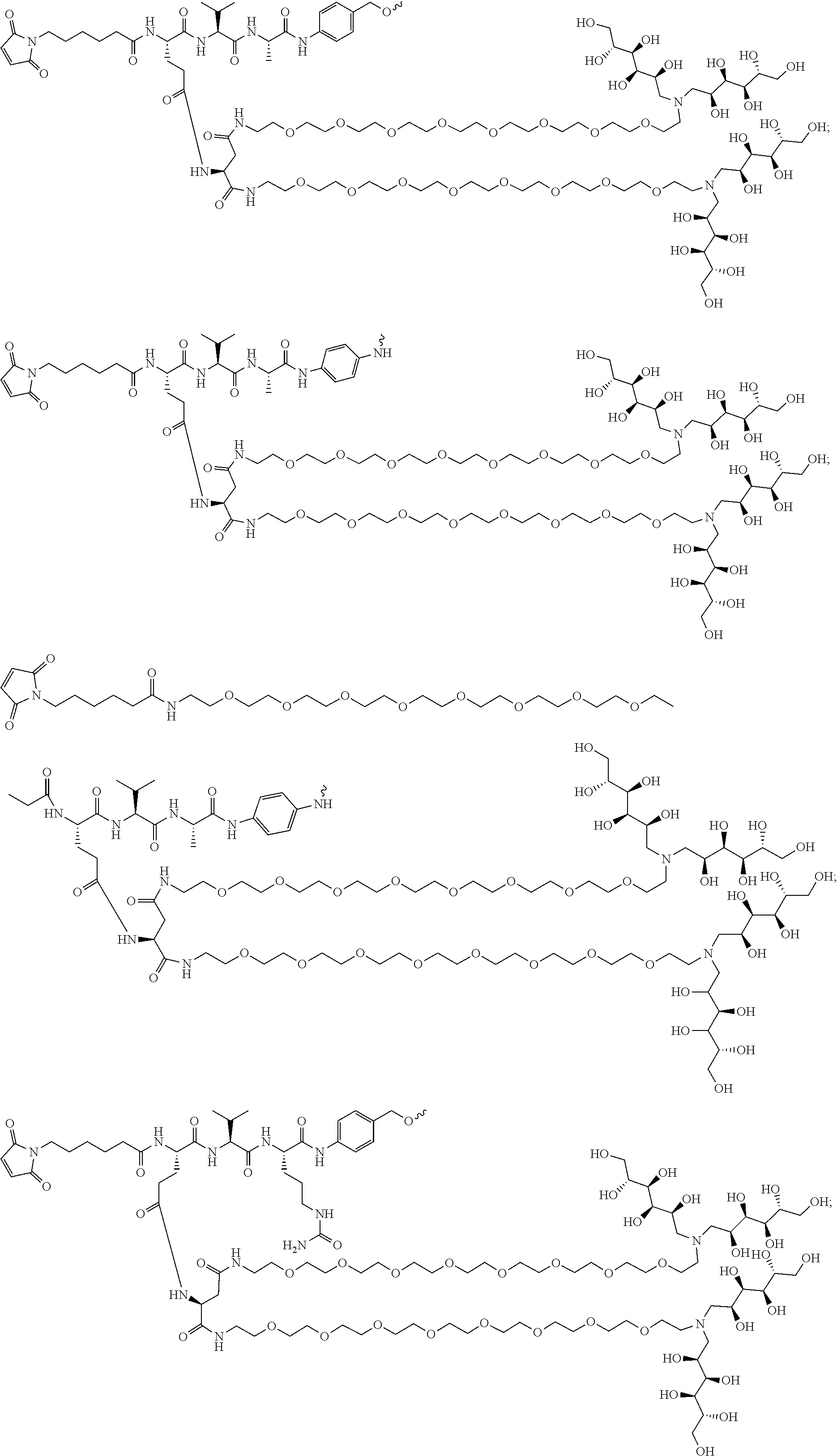

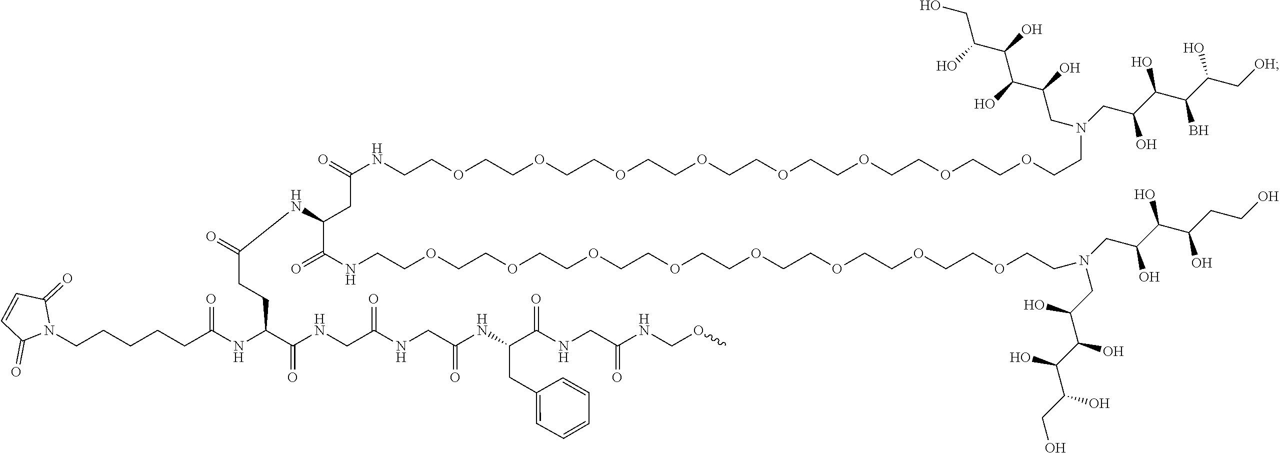

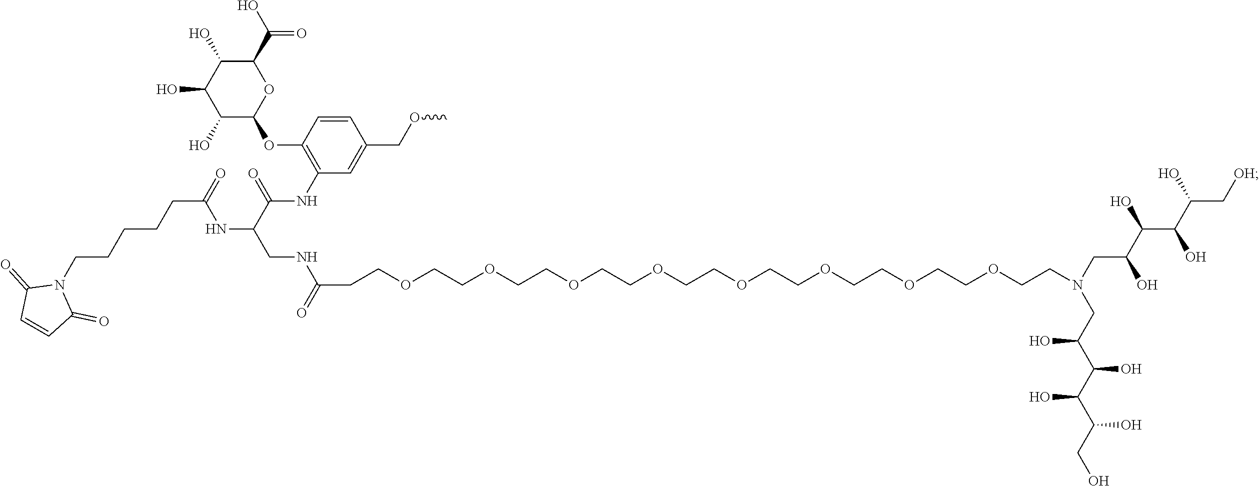

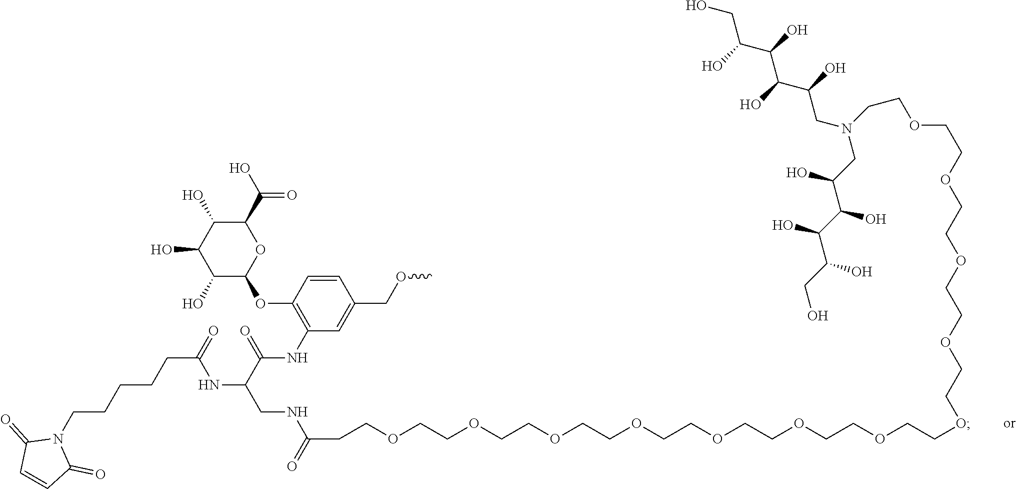

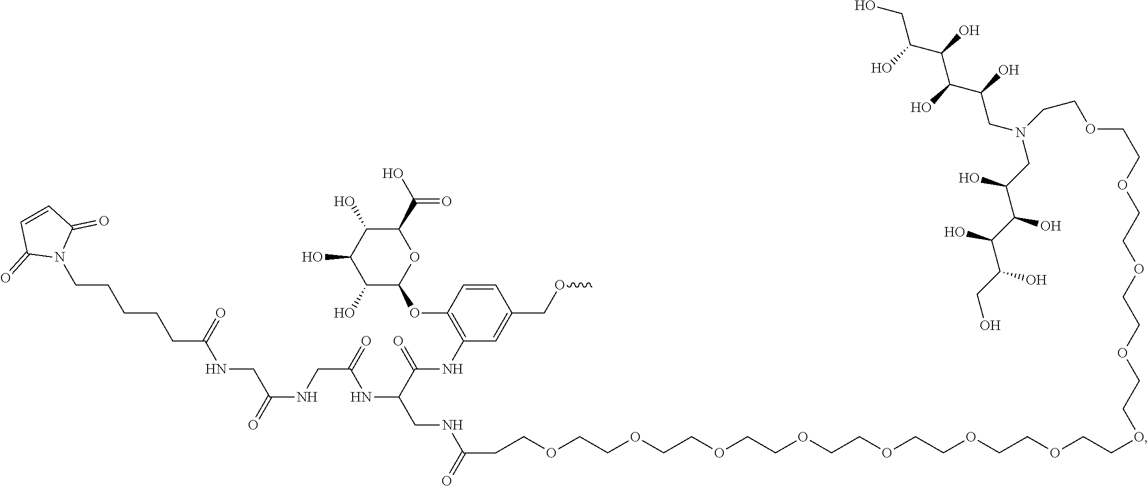

or a stereoisomer thereof, wherein the wavy line [symbol] indicates an attachment site to the drug units or for a linking group attached to the drug unit.

Embodiment 28. A drug-linker compound, comprising the linker compound of any one of Embodiments 1-27 attached to the drug unit, or attached to a linking group attached to the drug unit.

Embodiment 29. The drug-linker compound of Embodiment 28, wherein the drug unit is selected from a cytotoxic agent, an immune modulatory agent, a nucleic acid, a growth inhibitory agent, a PROTAC, a toxin, a radioactive isotope and a chelating ligand.

Embodiment 30. A conjugate, comprising the drug-linker compound of Embodiment 29, wherein the drug-linker compound is attached to a binding agent (e.g., EGFR and c-MET binding agent).

Embodiment 31. The conjugate of Embodiment 30, wherein an average drug loading (p^{load}) of the conjugate is from about 1 to about 8, about 2, about 3, about 4, about 5, about 6, about 7, about 8, about 10, about 12, about 14, about 16, about 3 to about 5, about 6 to about 8, or about 8 to about 16.

Embodiment 32. The conjugate of Embodiment 30 or 31, selected from the following:

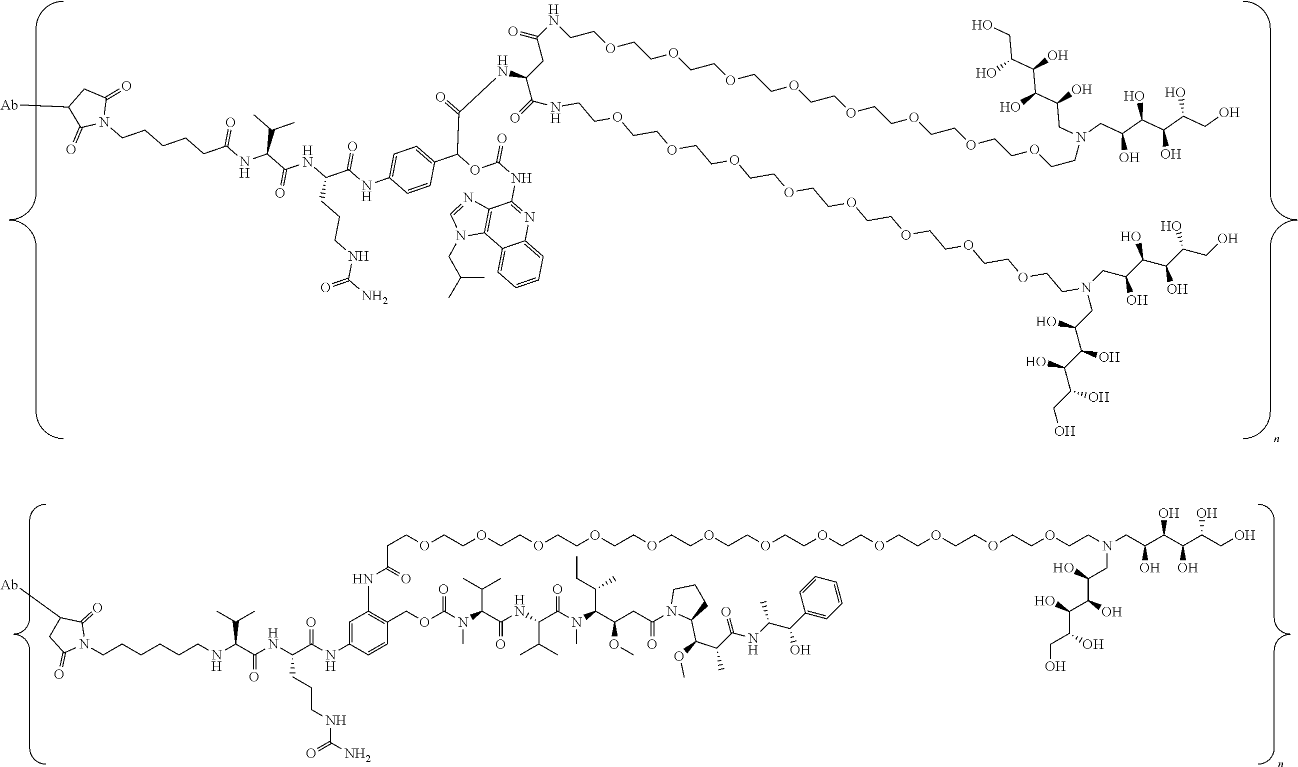

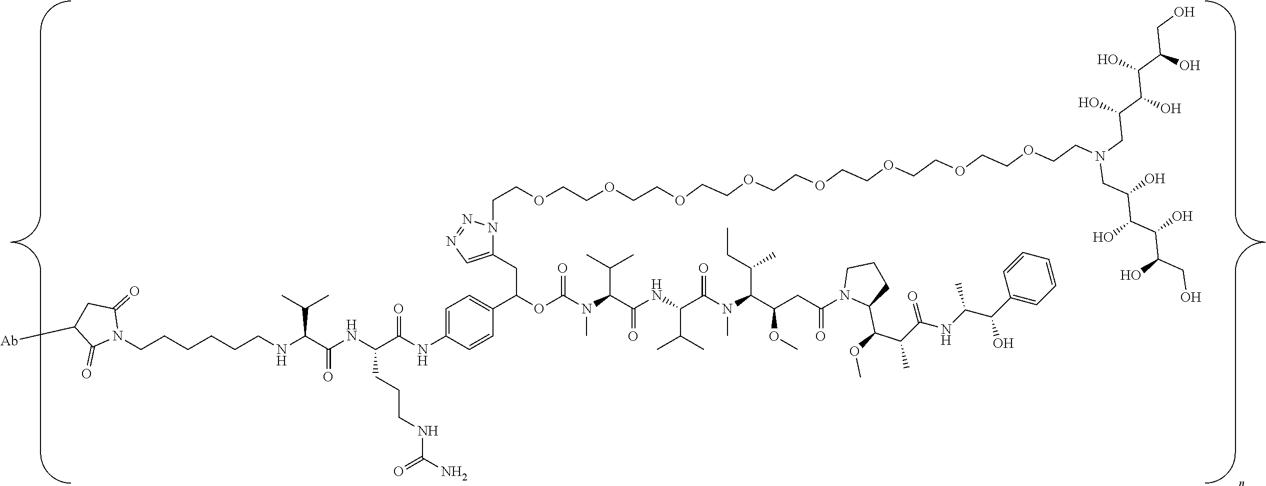

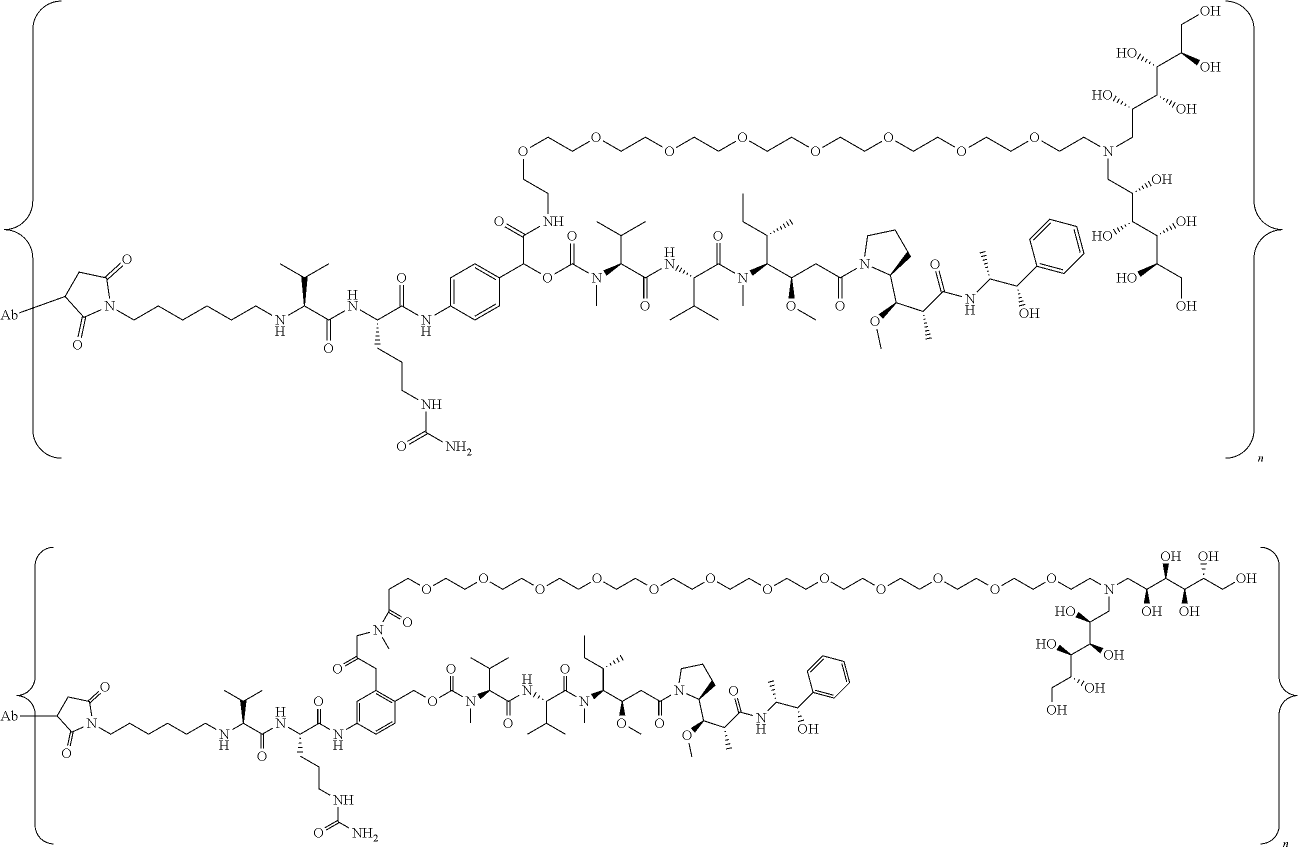

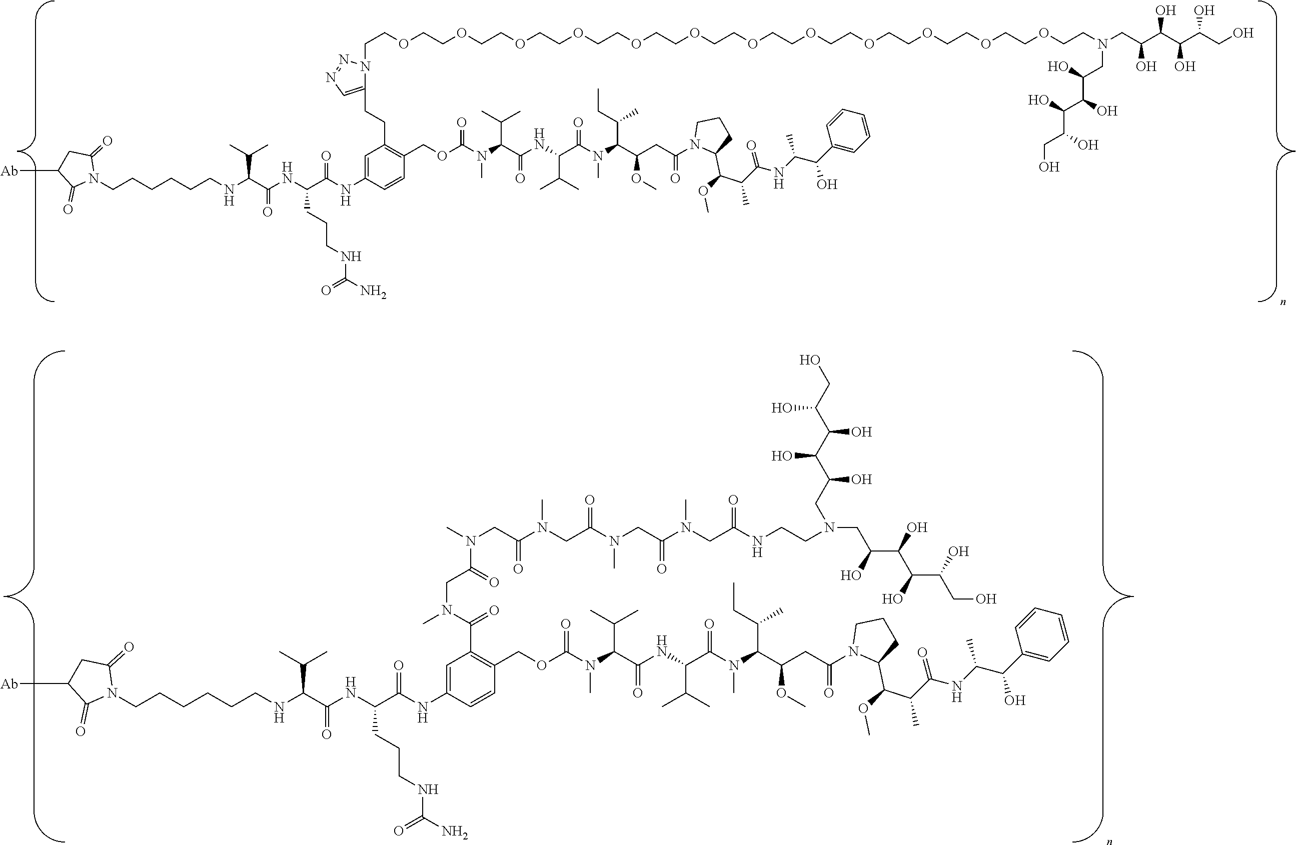

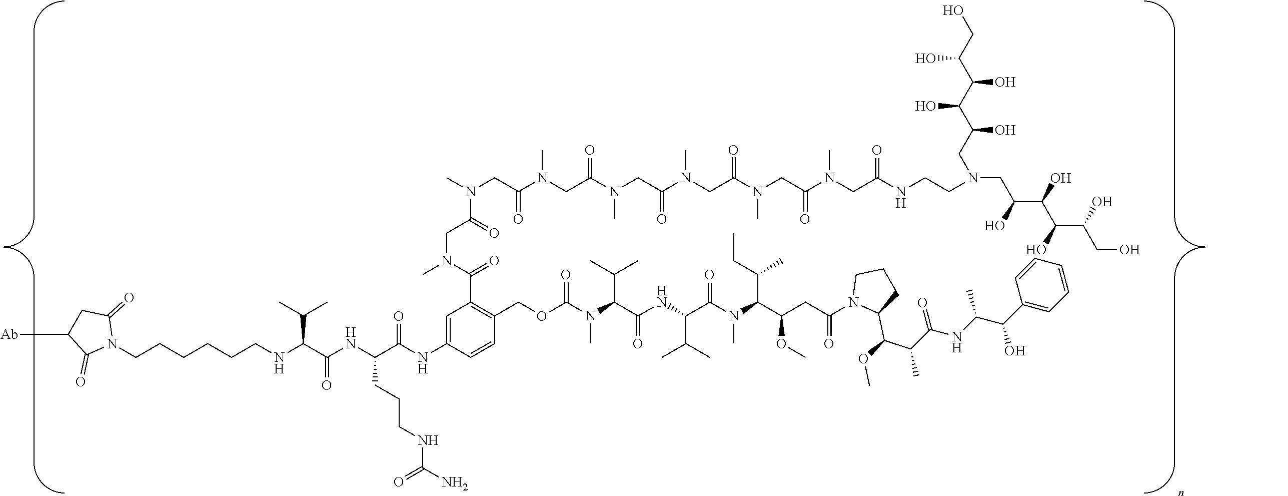

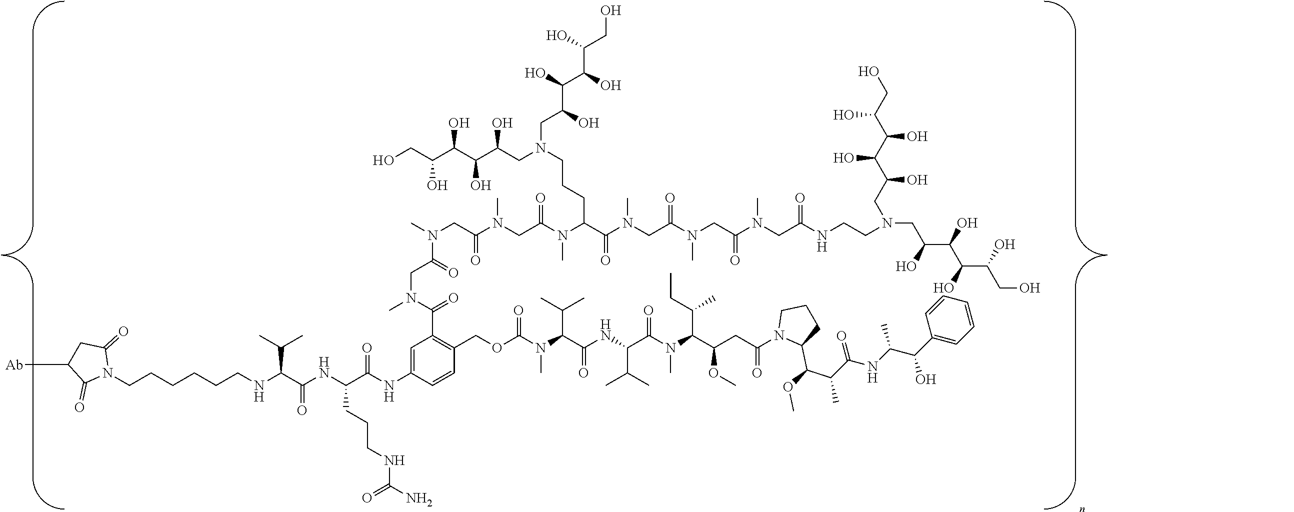

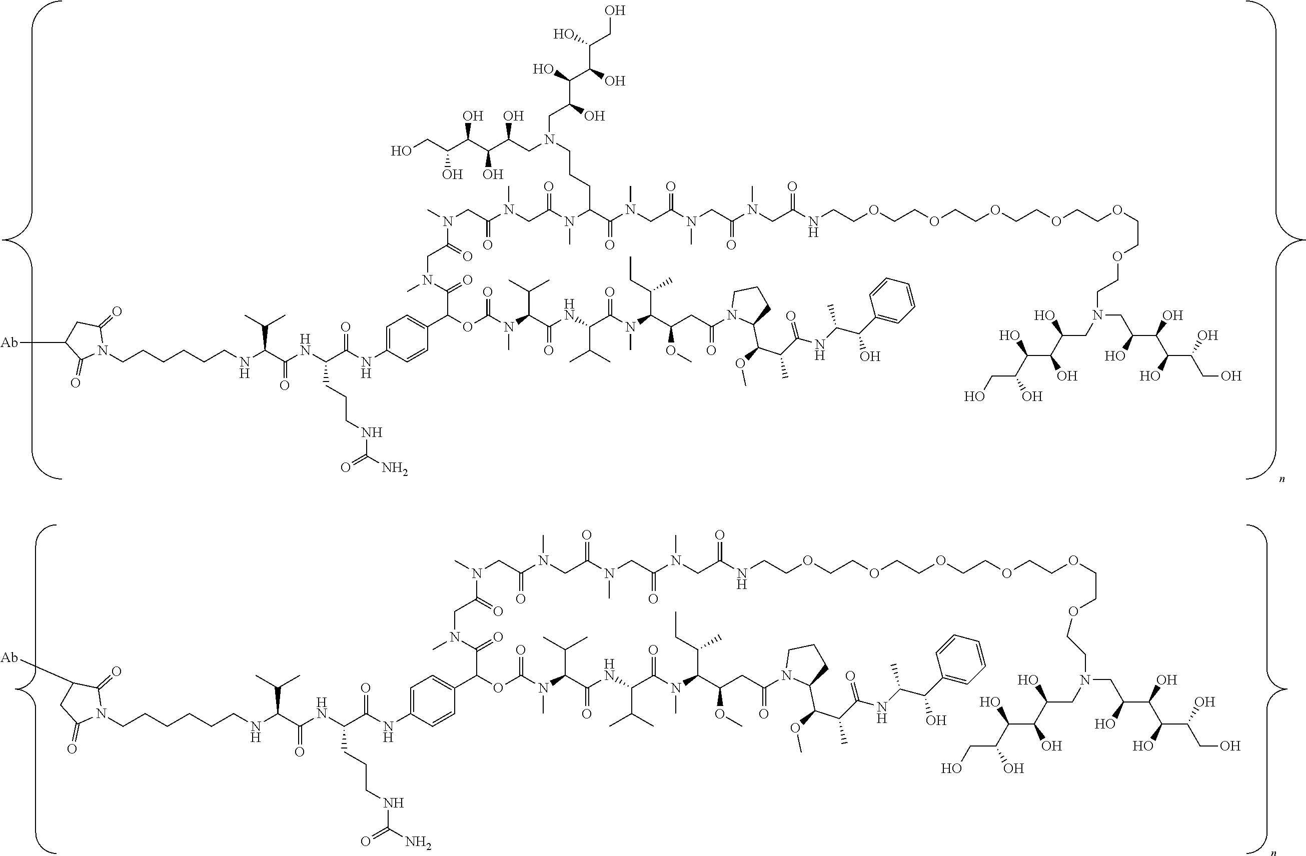

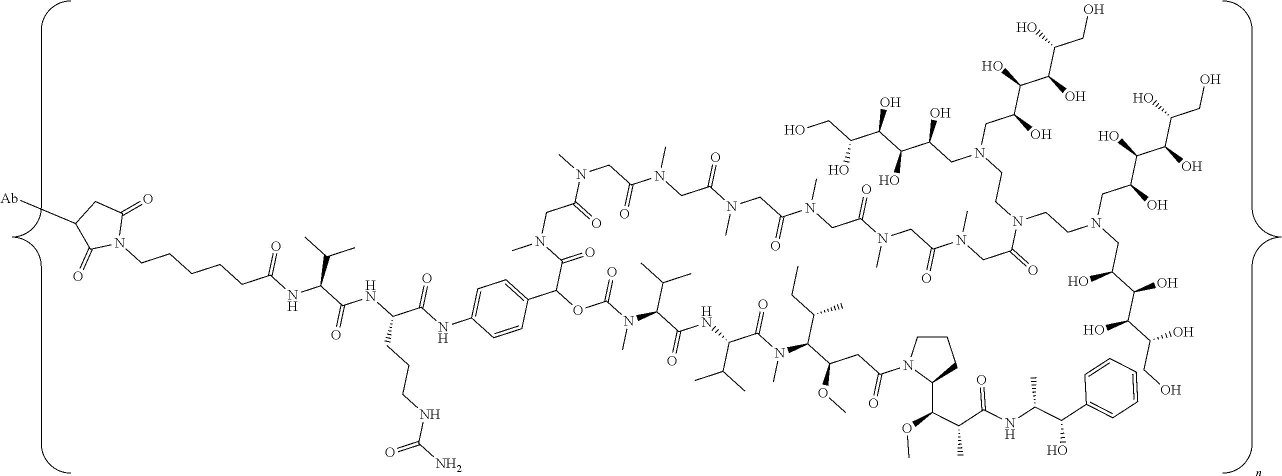

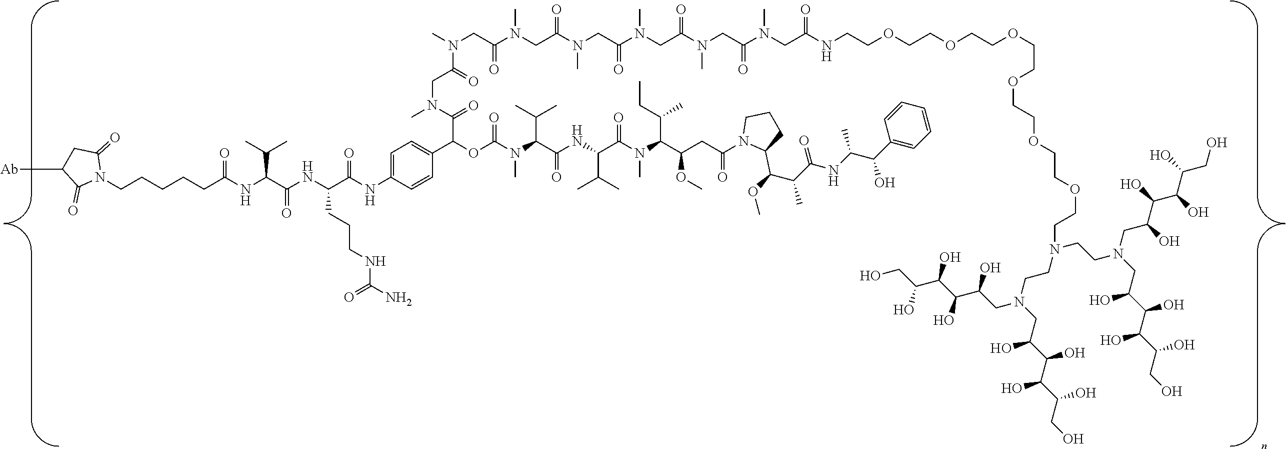

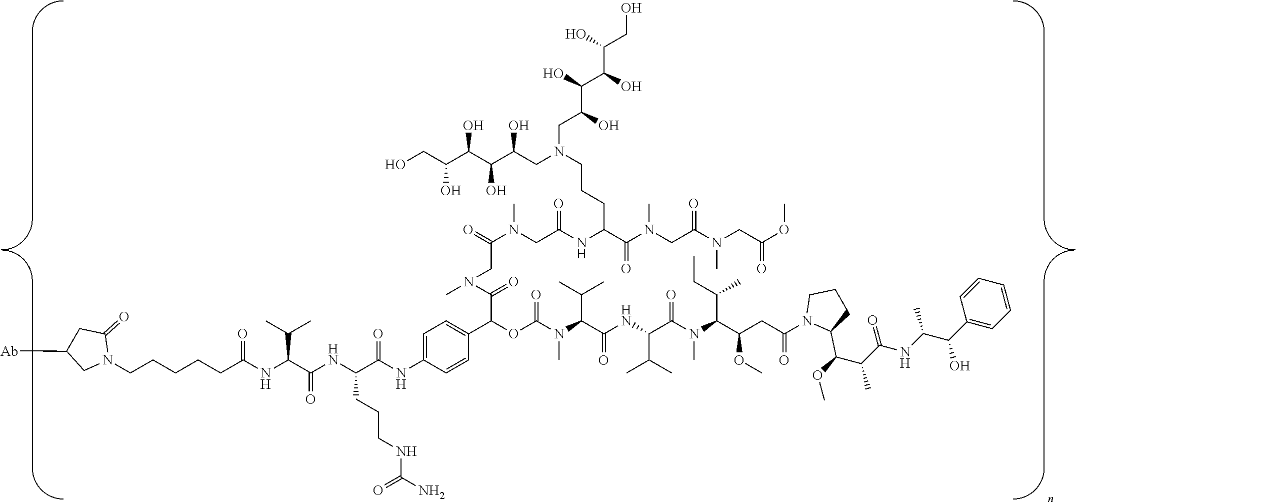

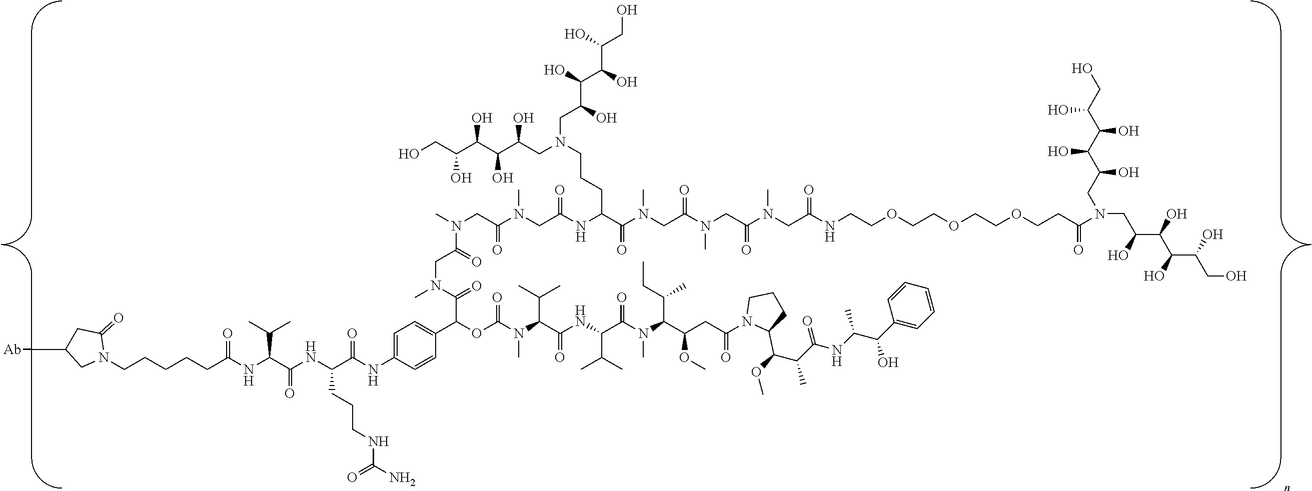

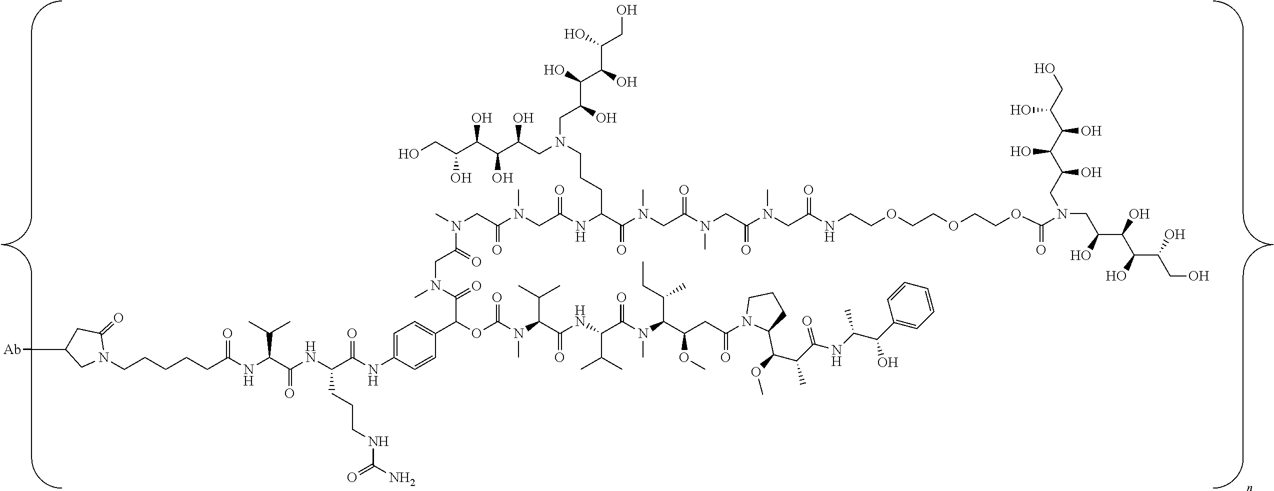

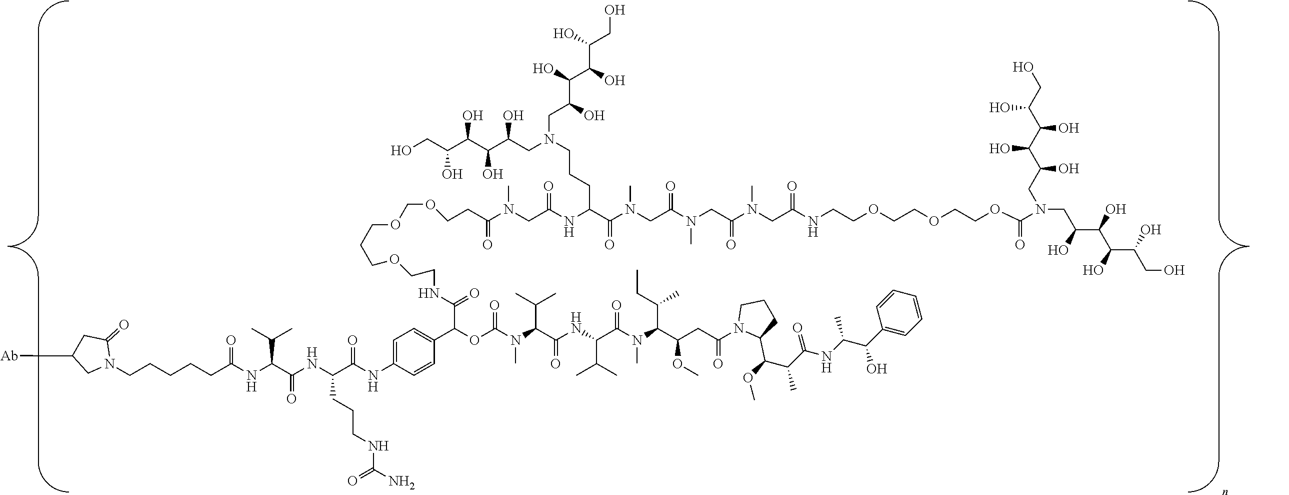

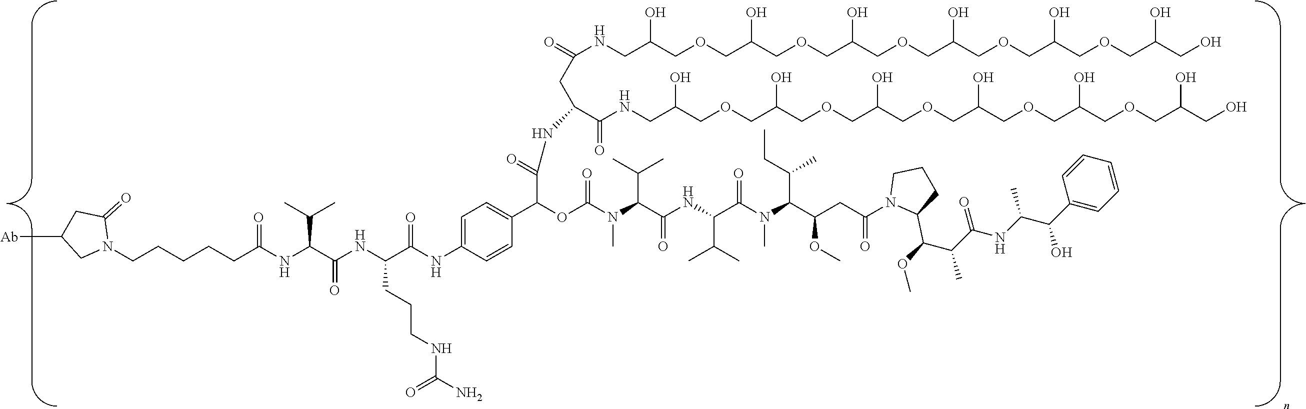

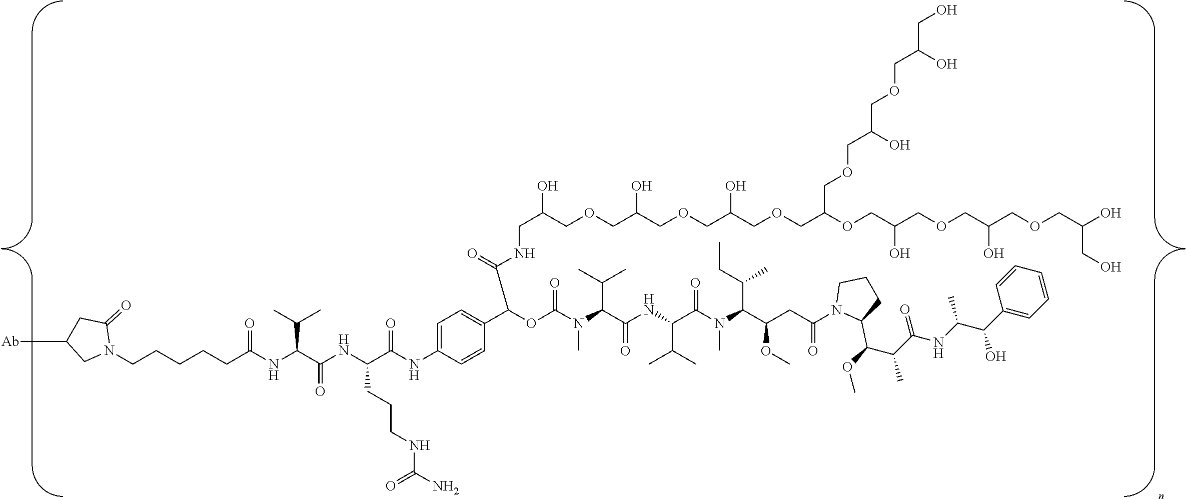

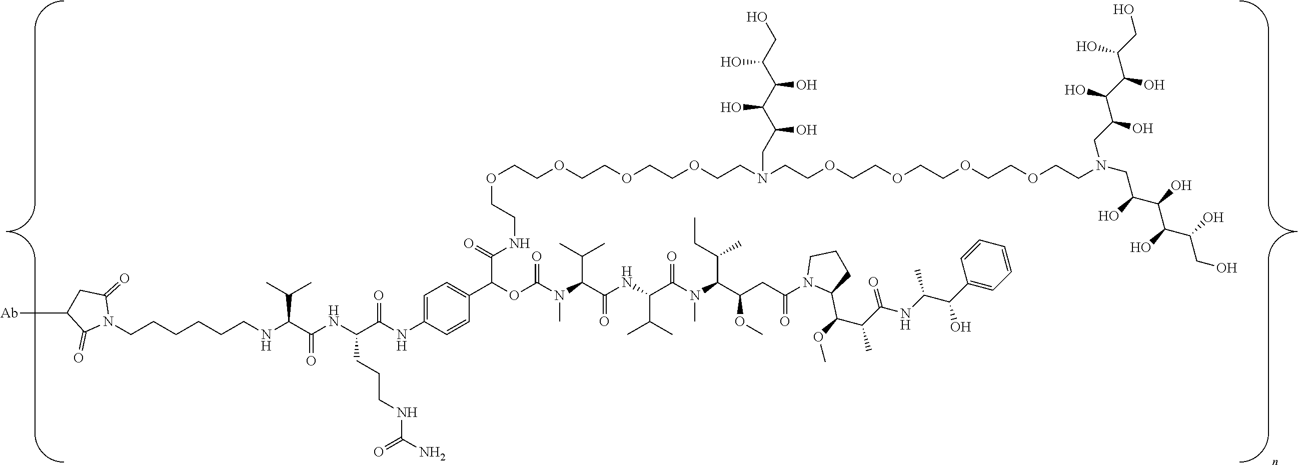

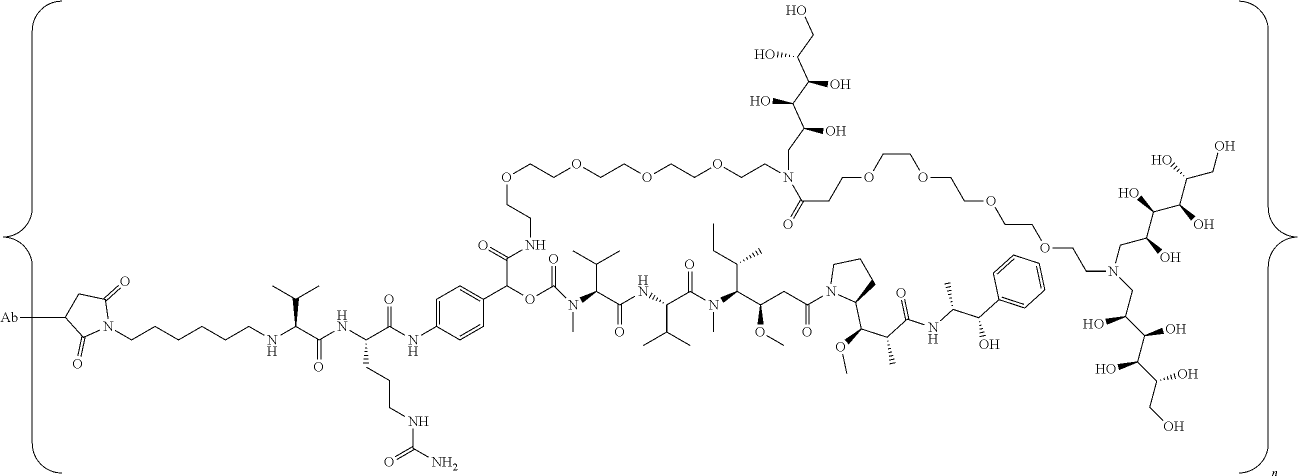

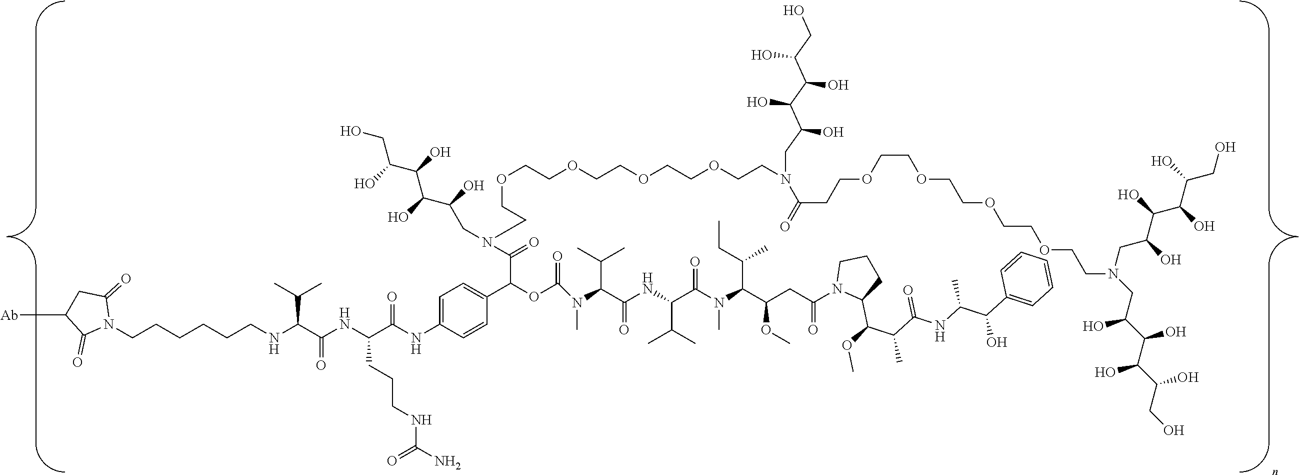

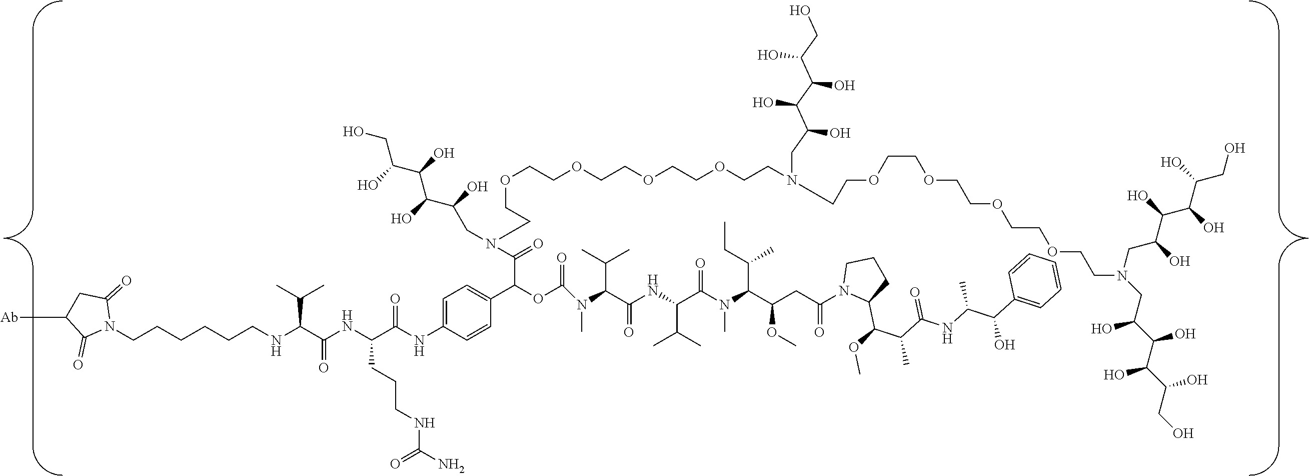

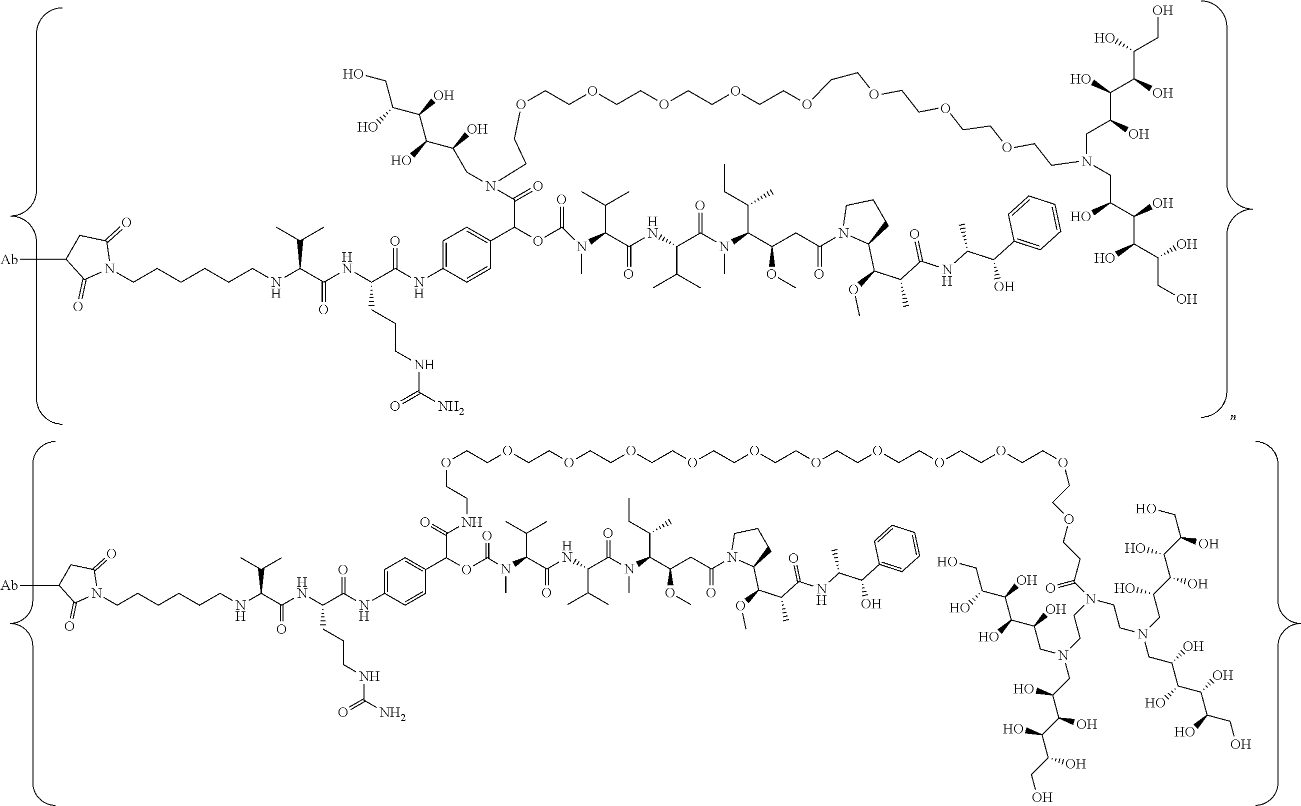

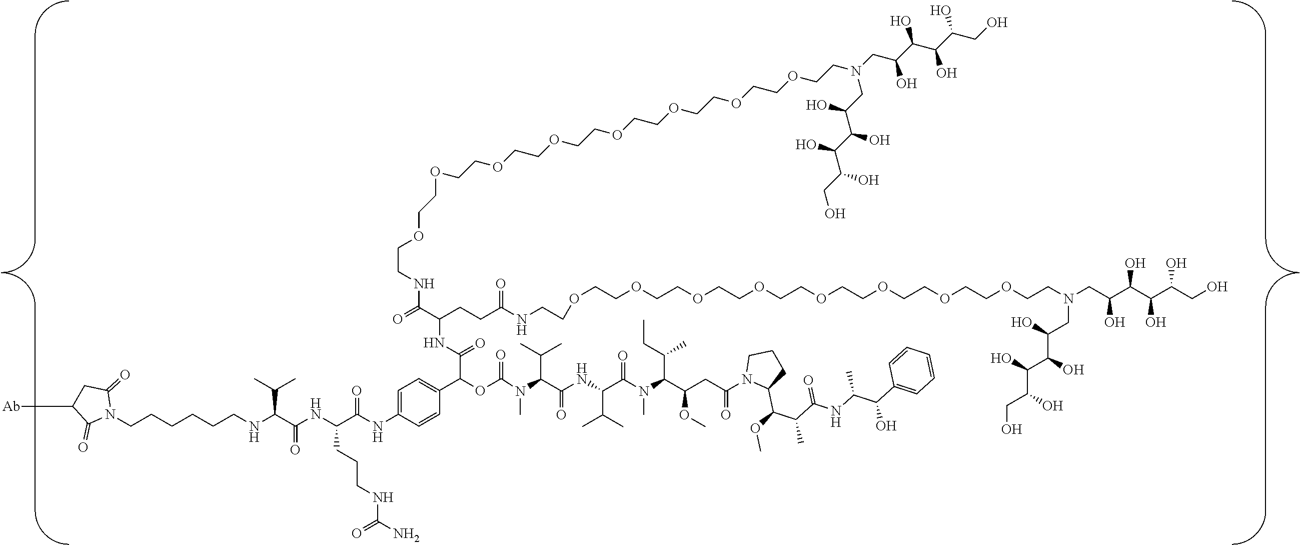

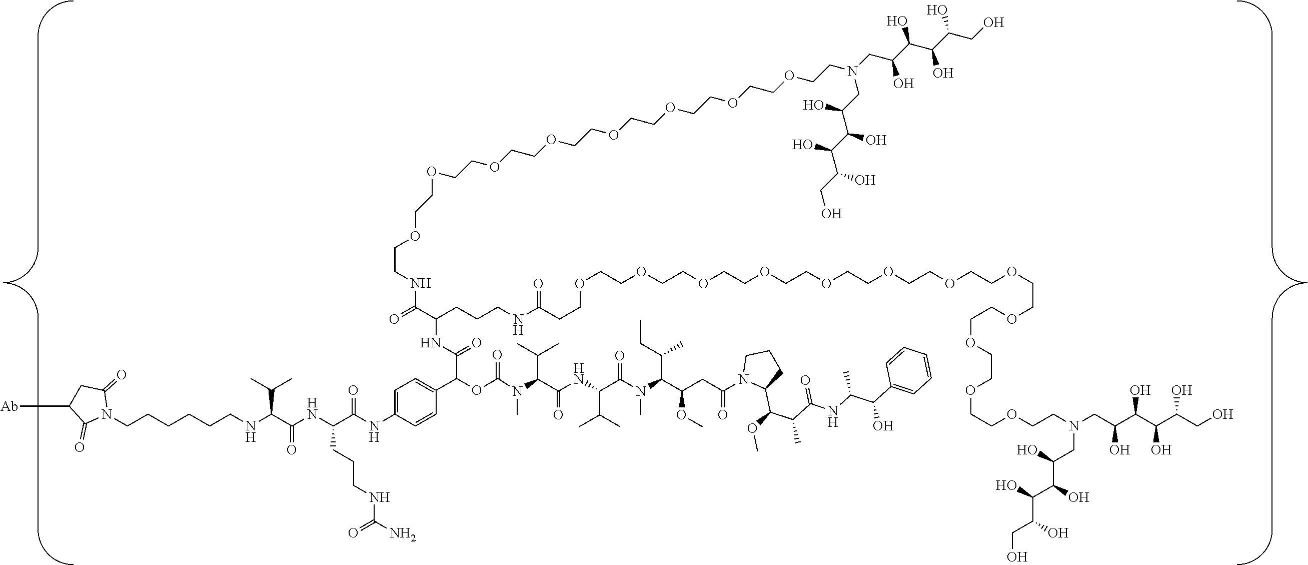

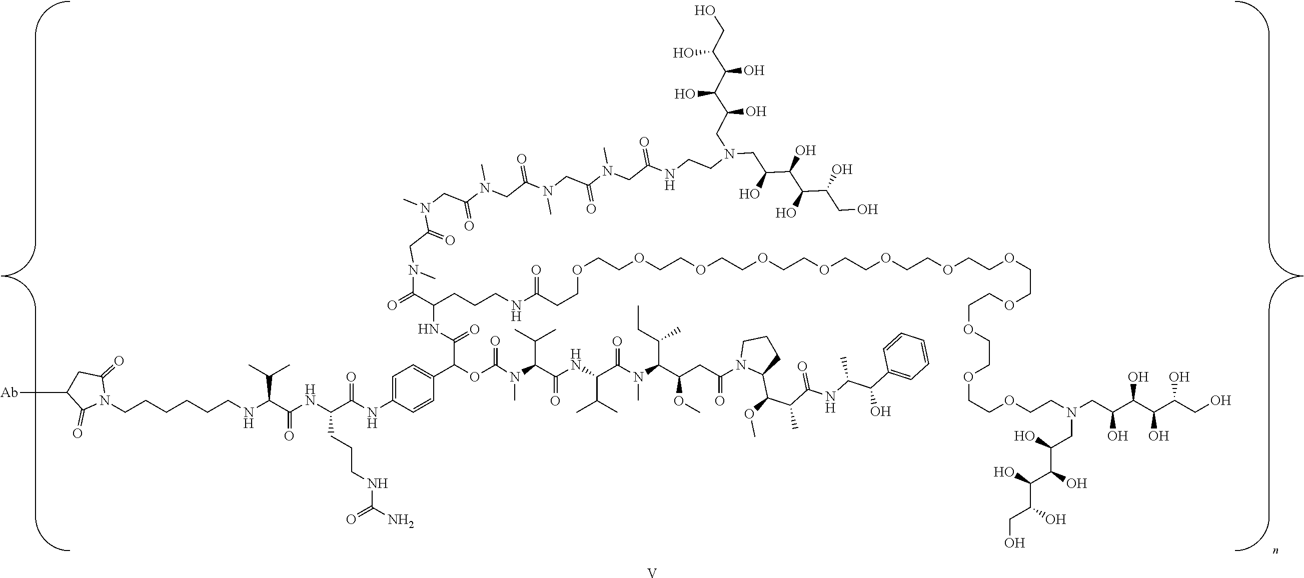

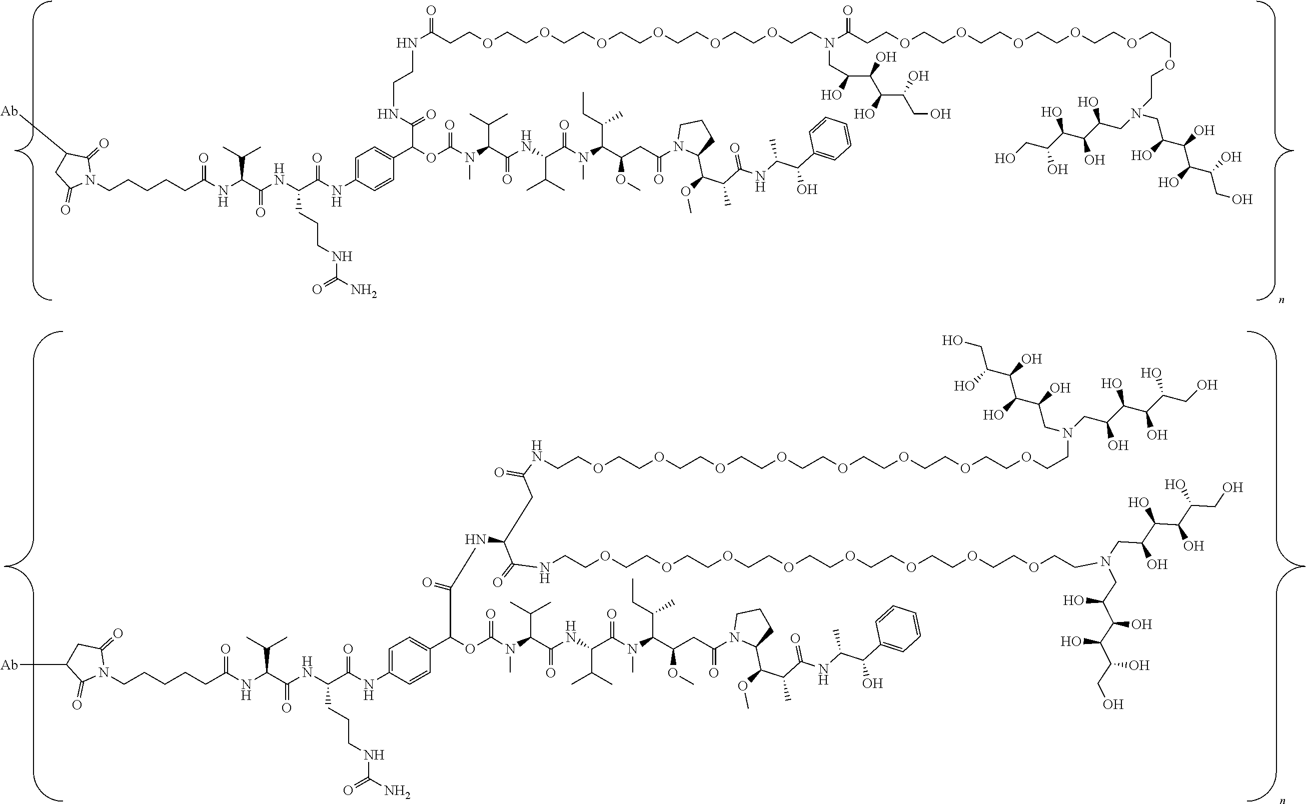

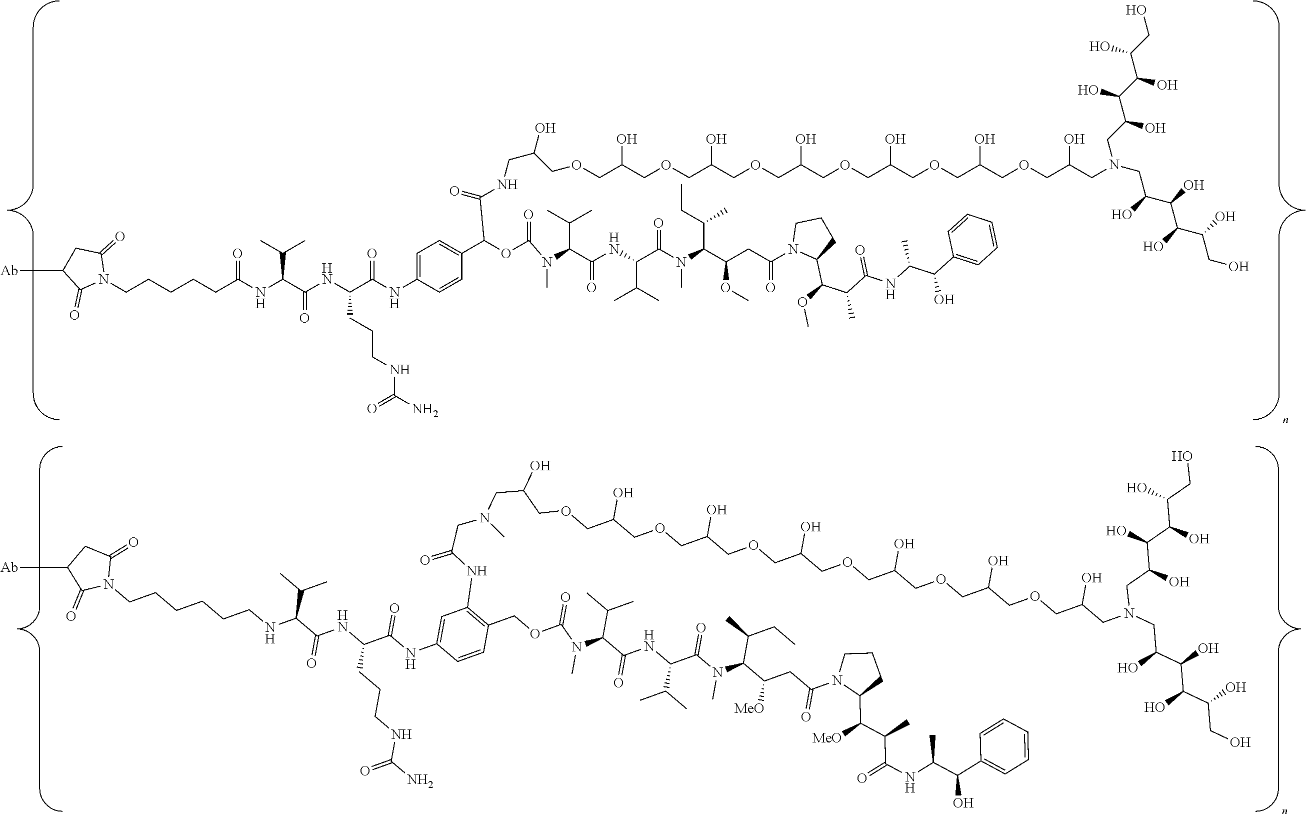

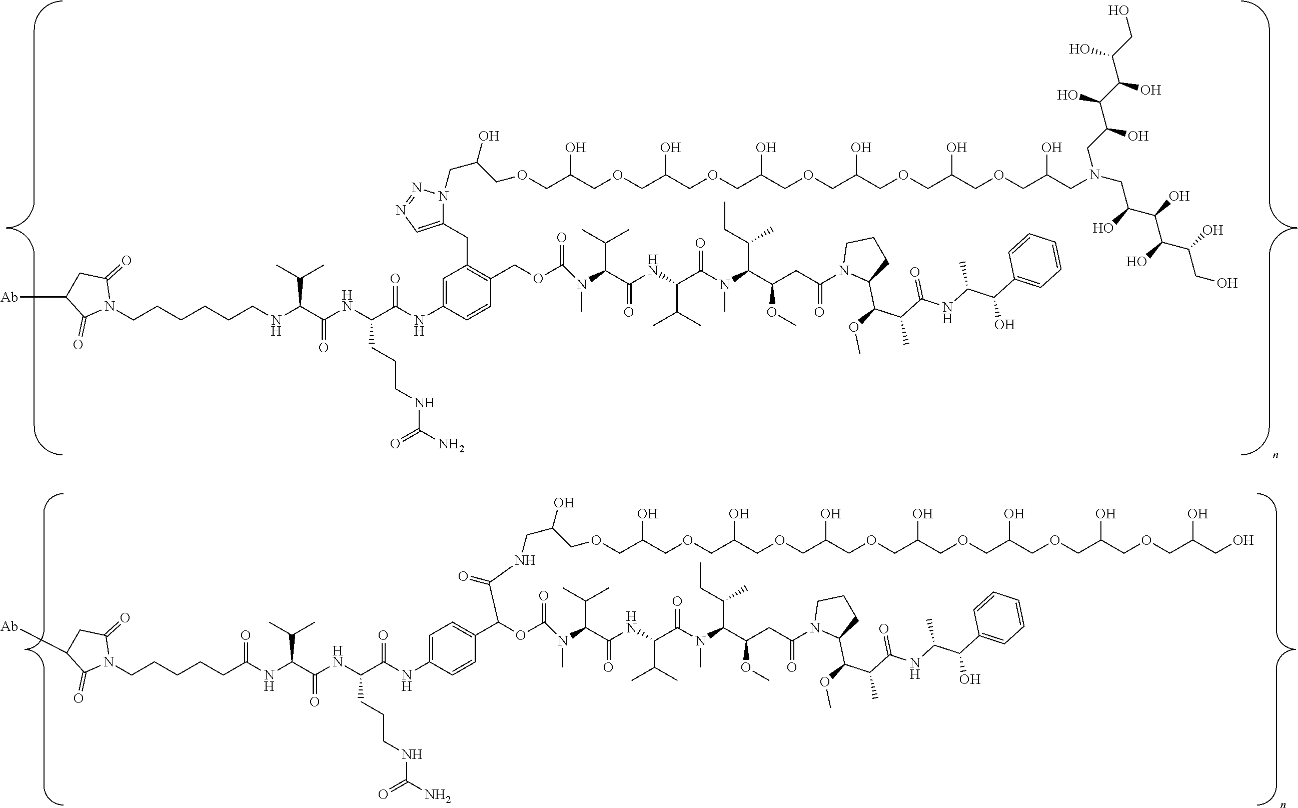

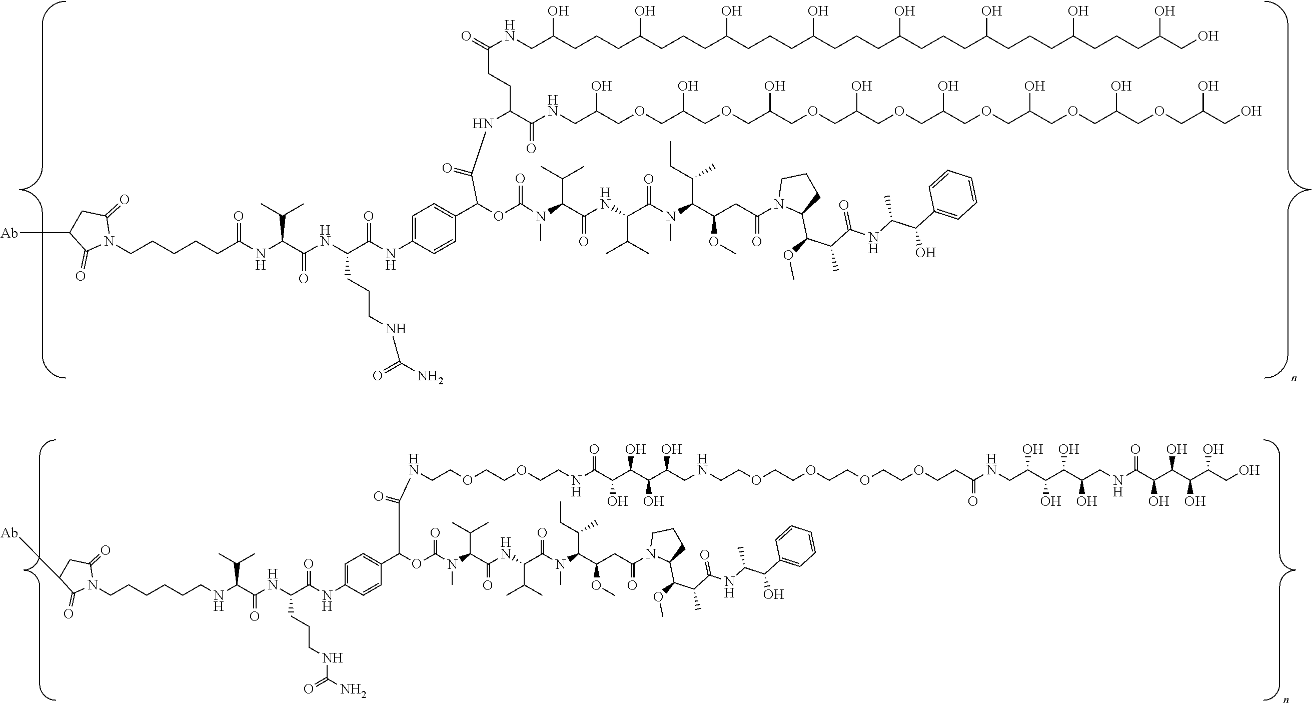

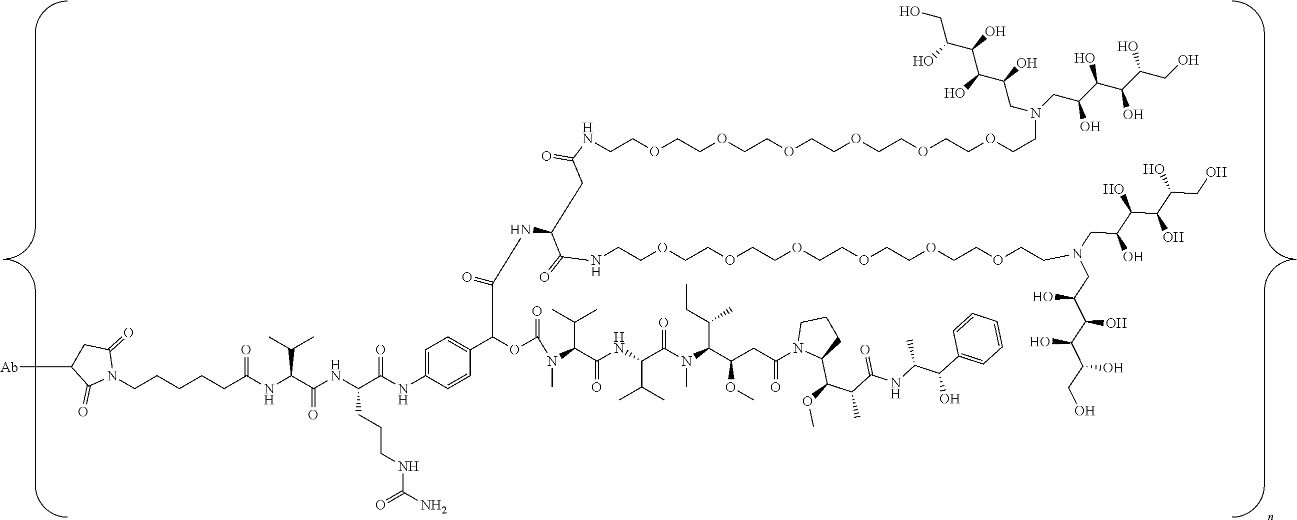

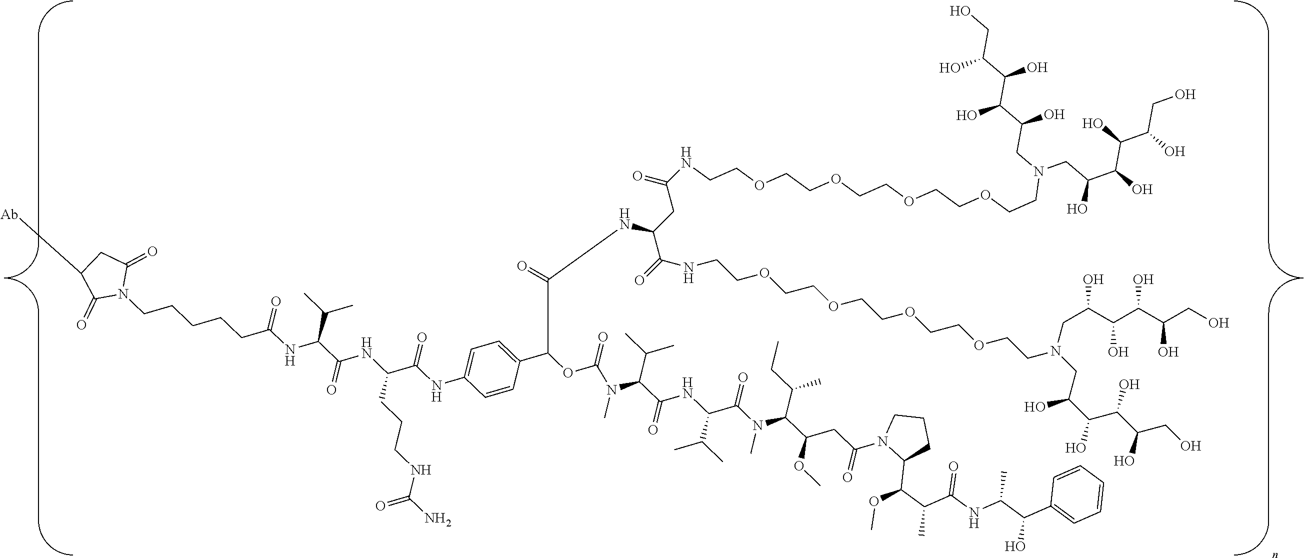

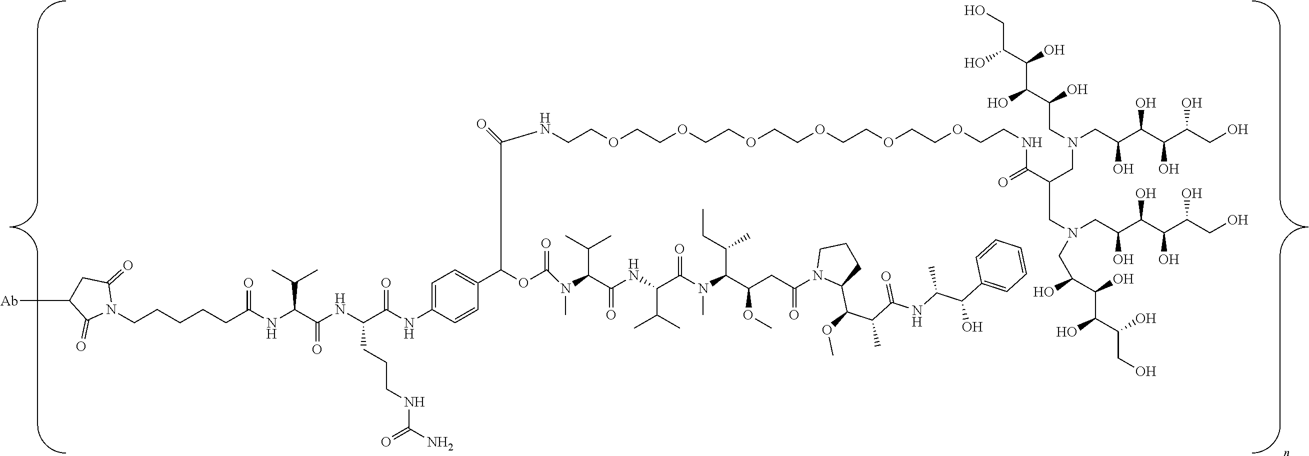

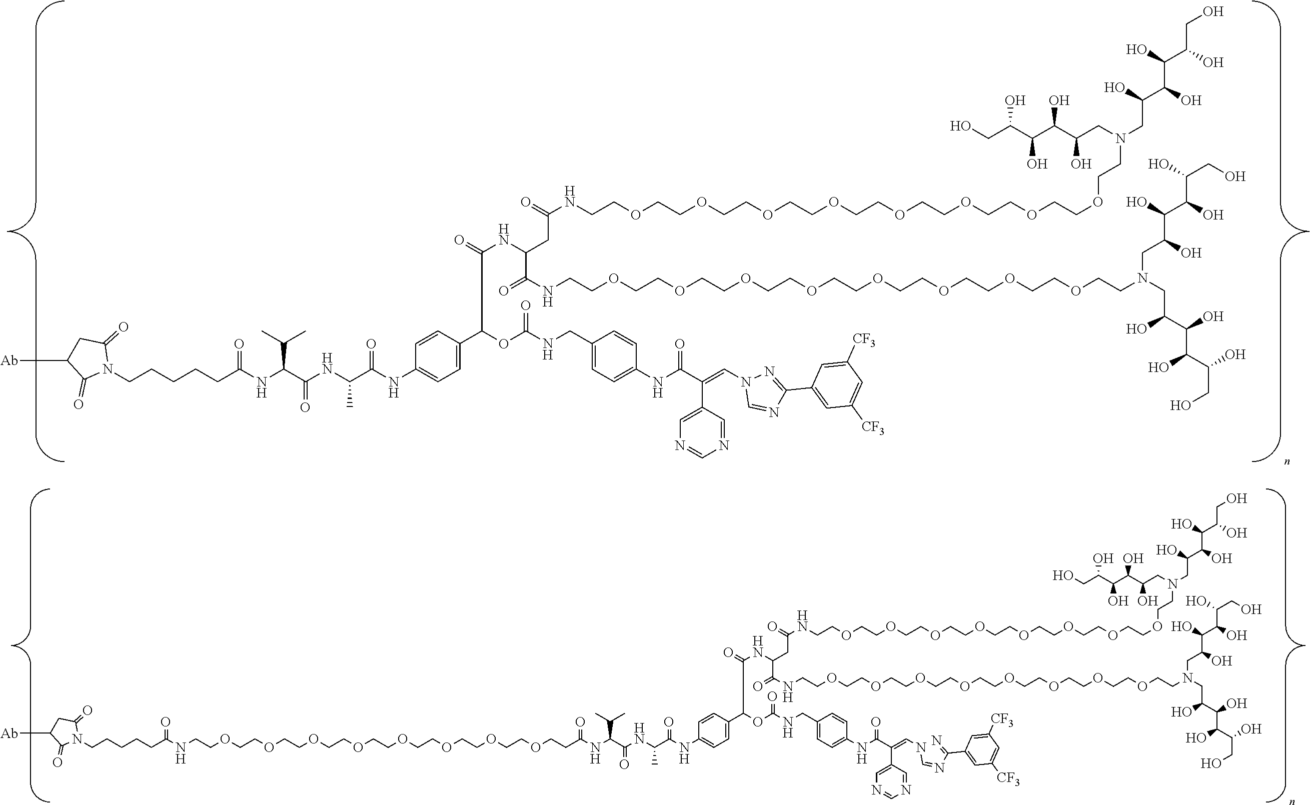

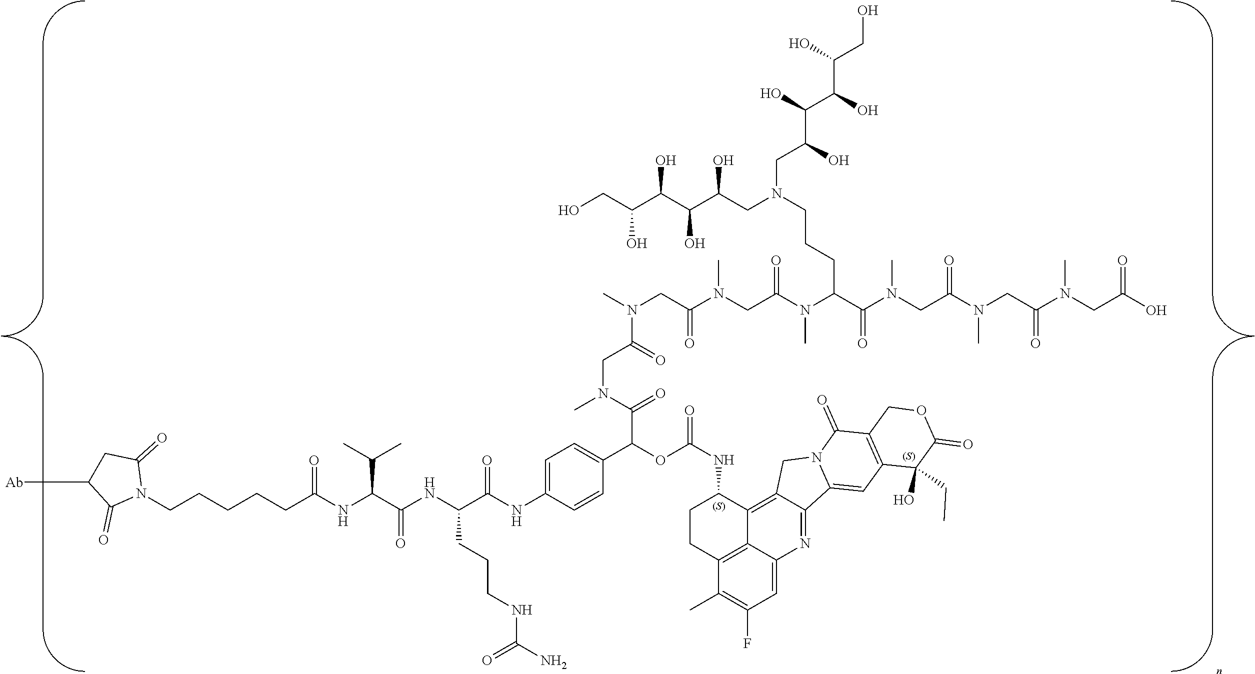

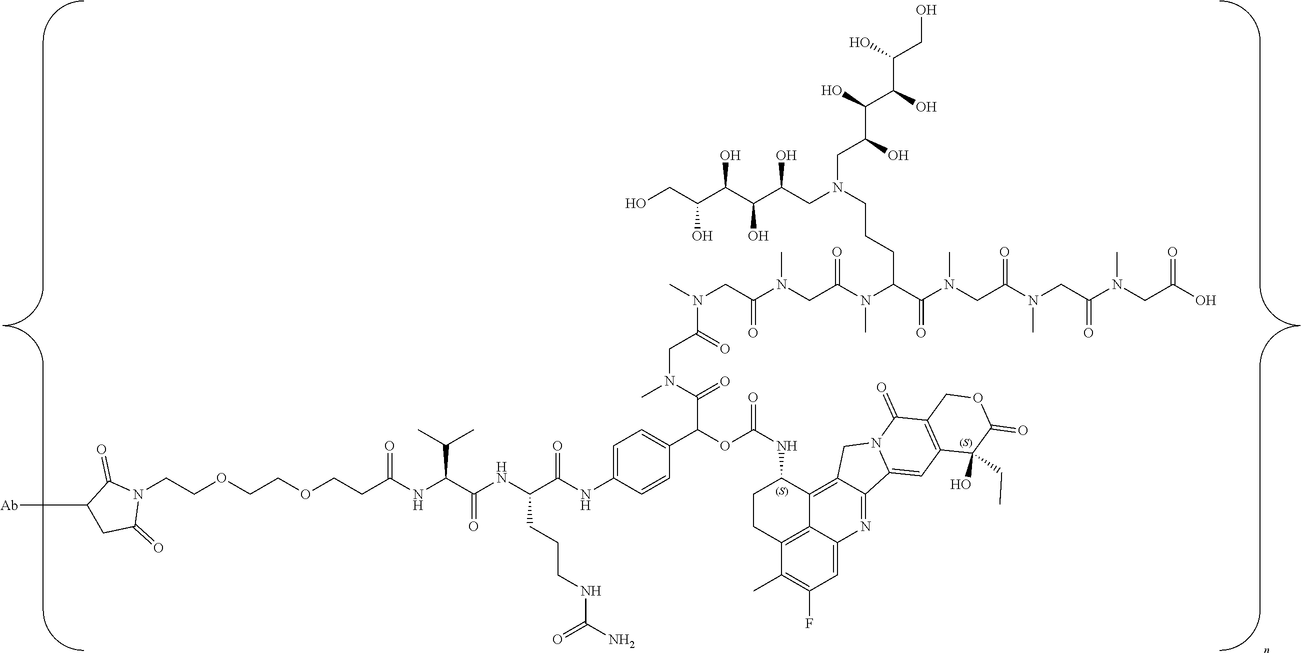

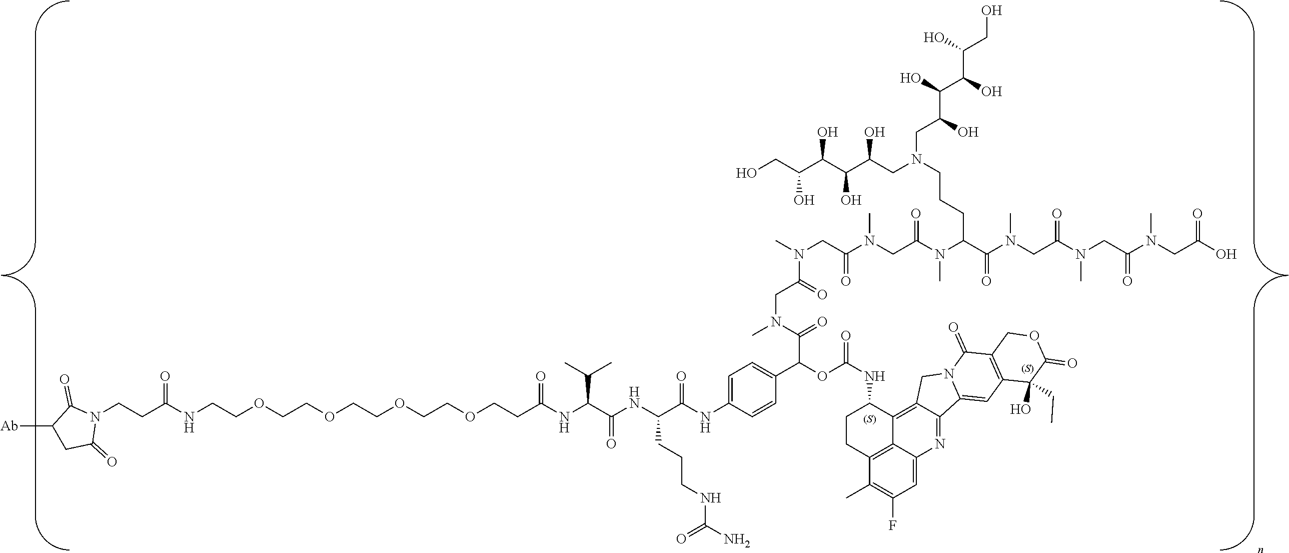

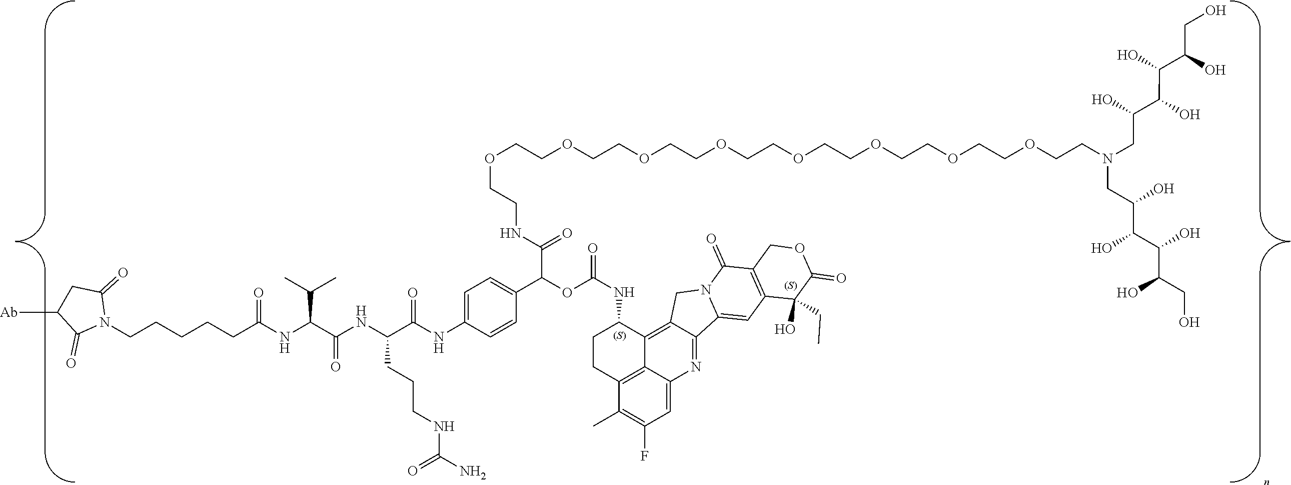

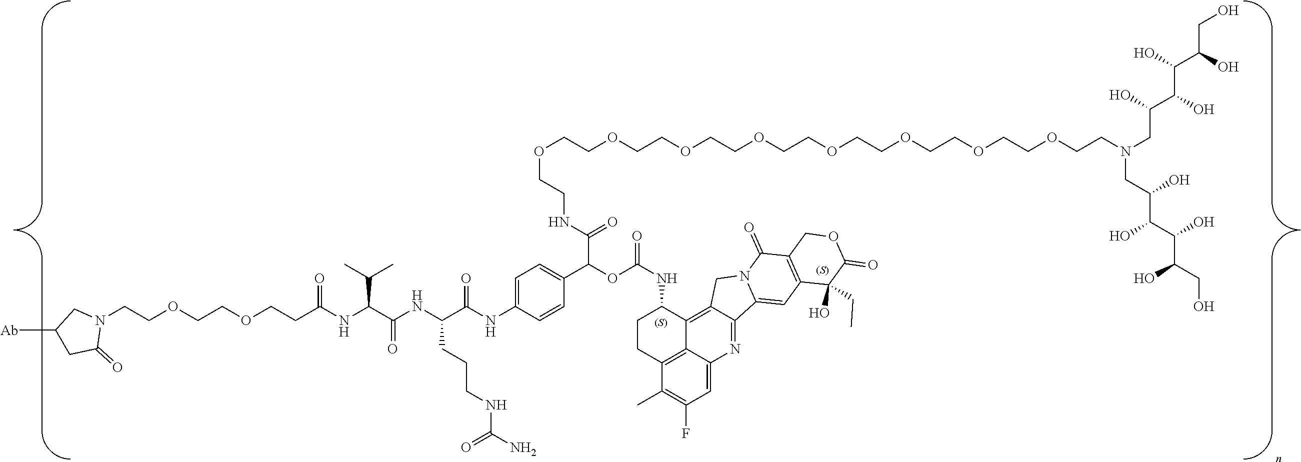

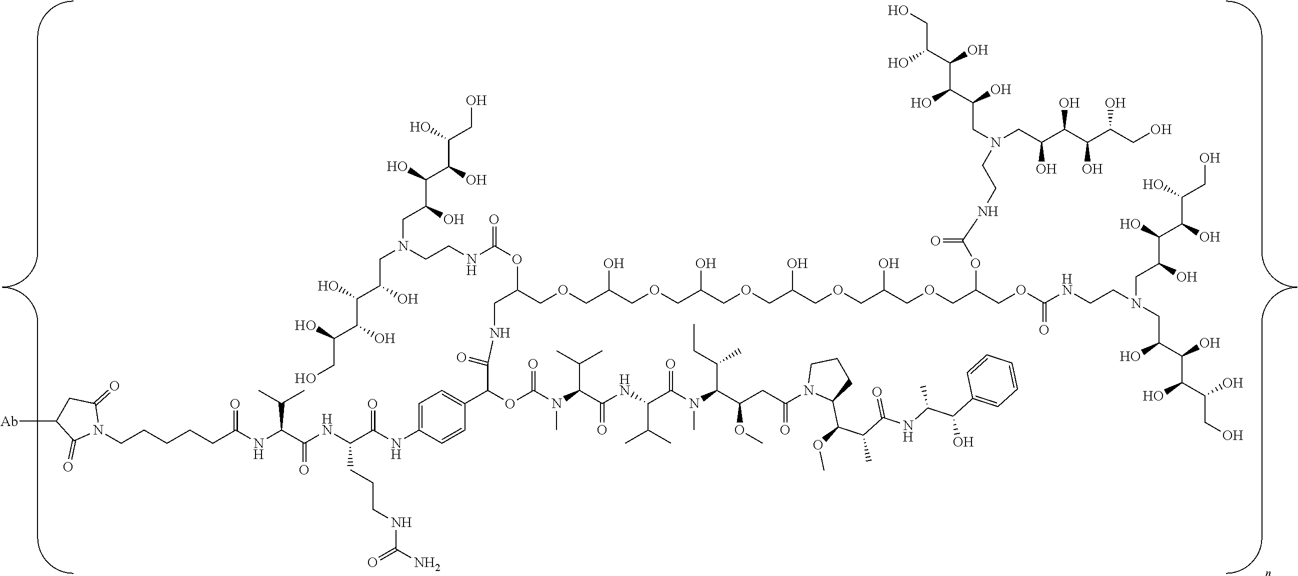

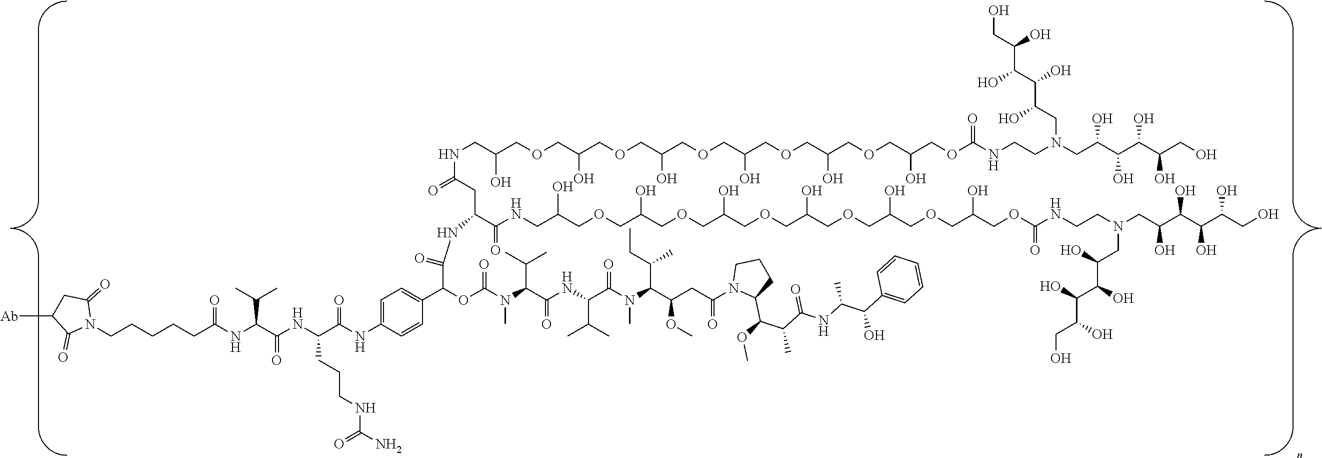

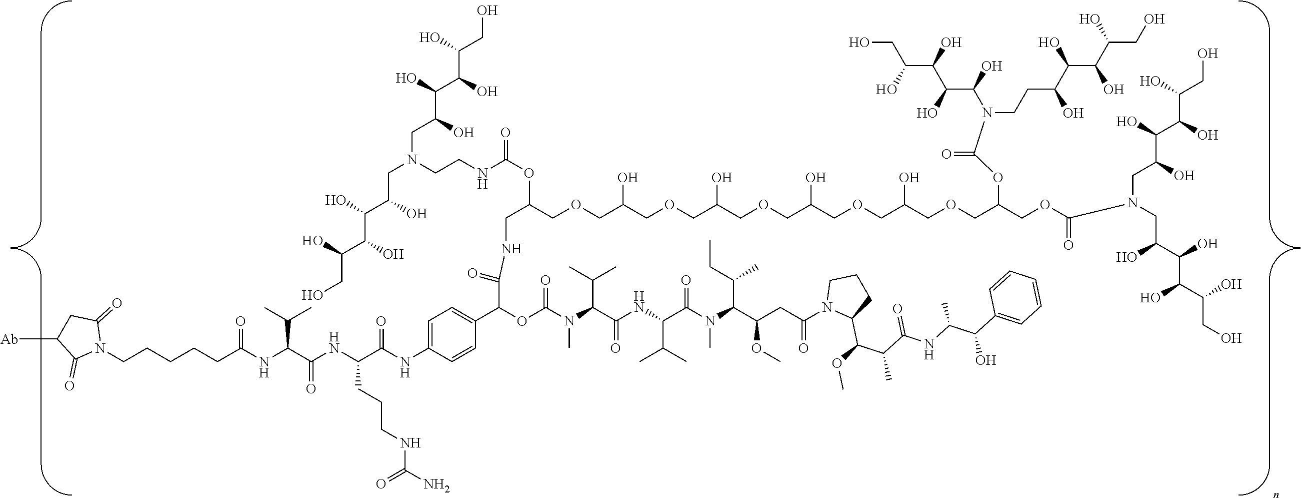

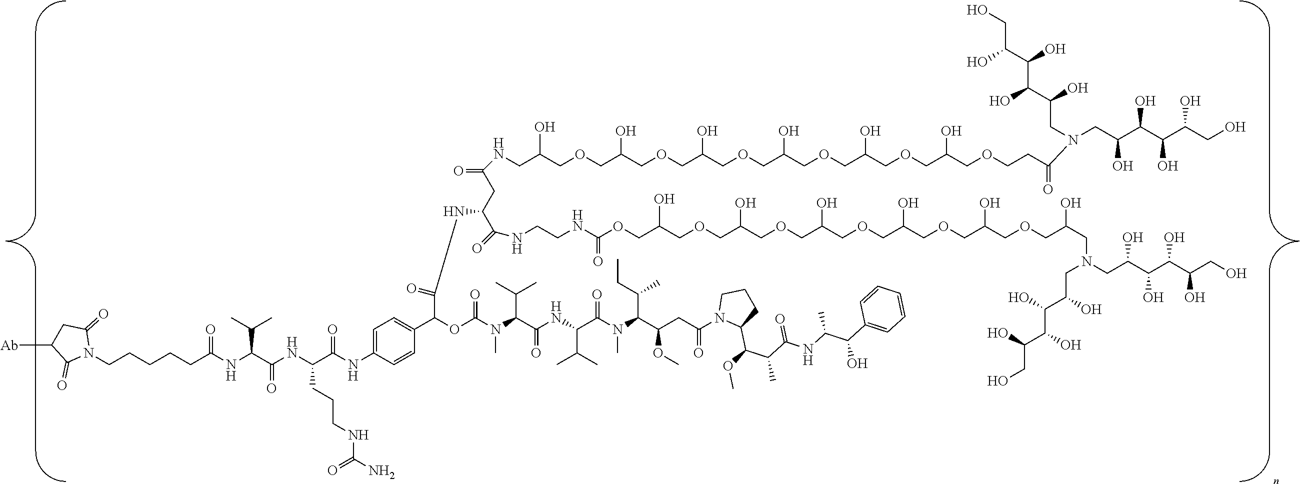

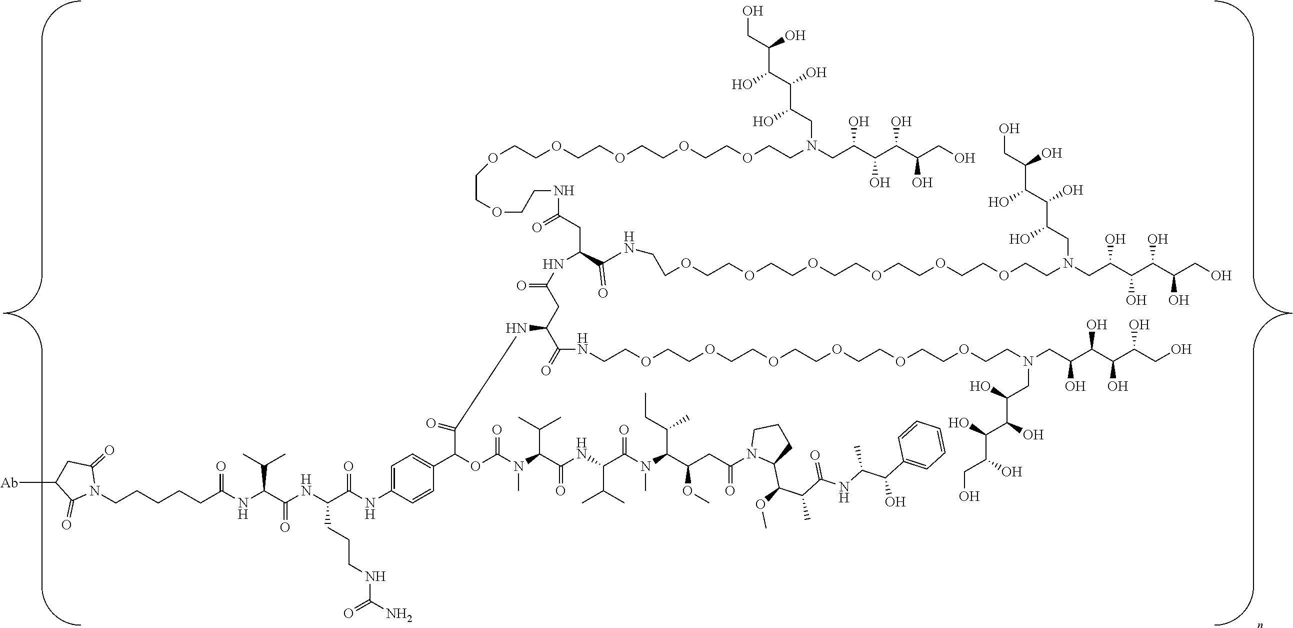

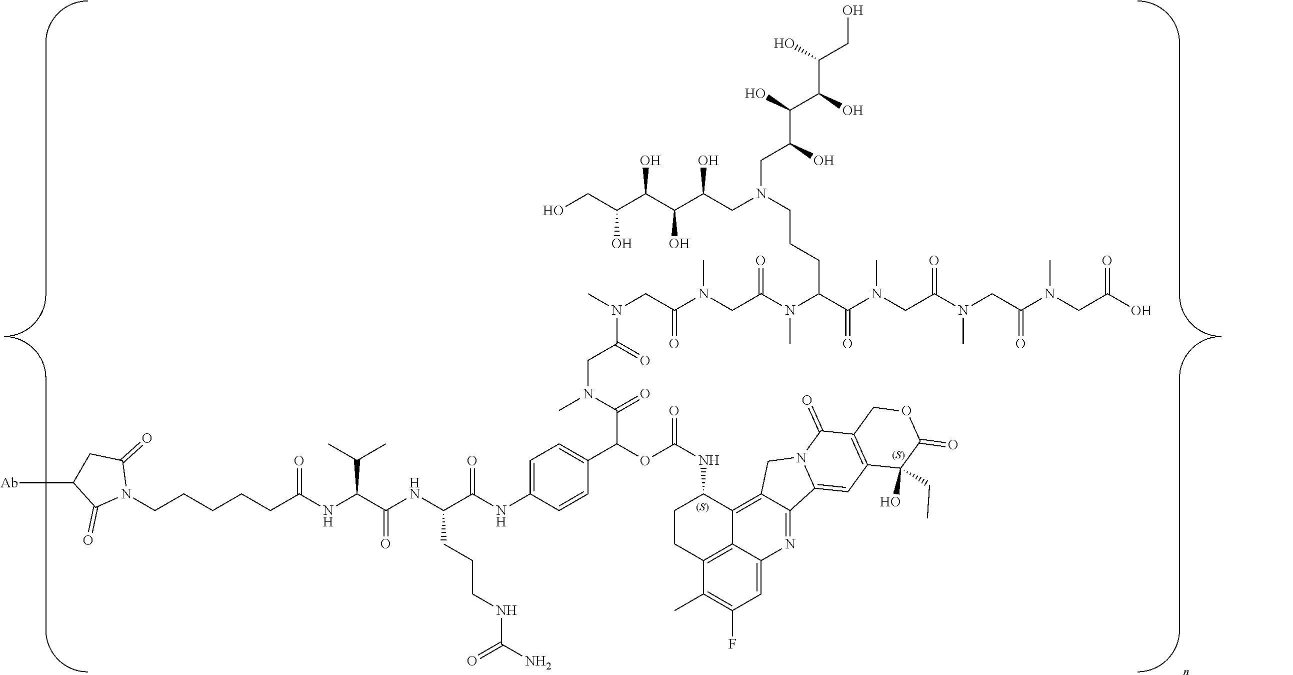

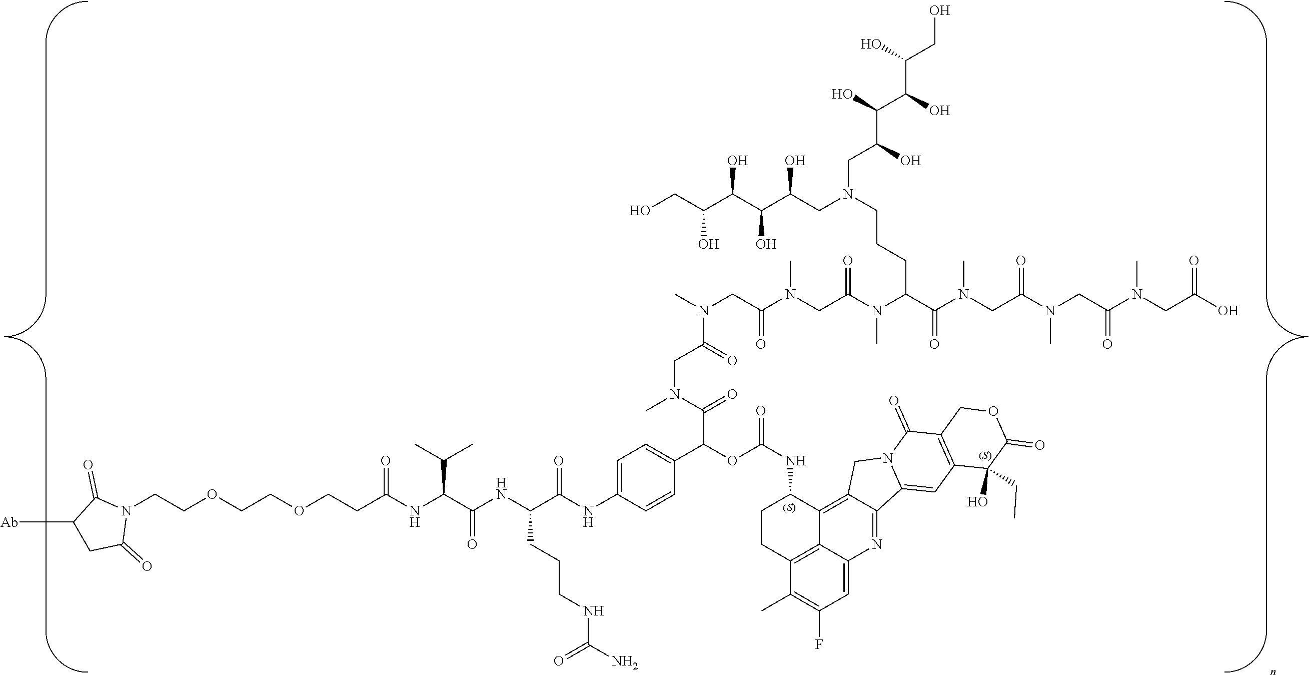

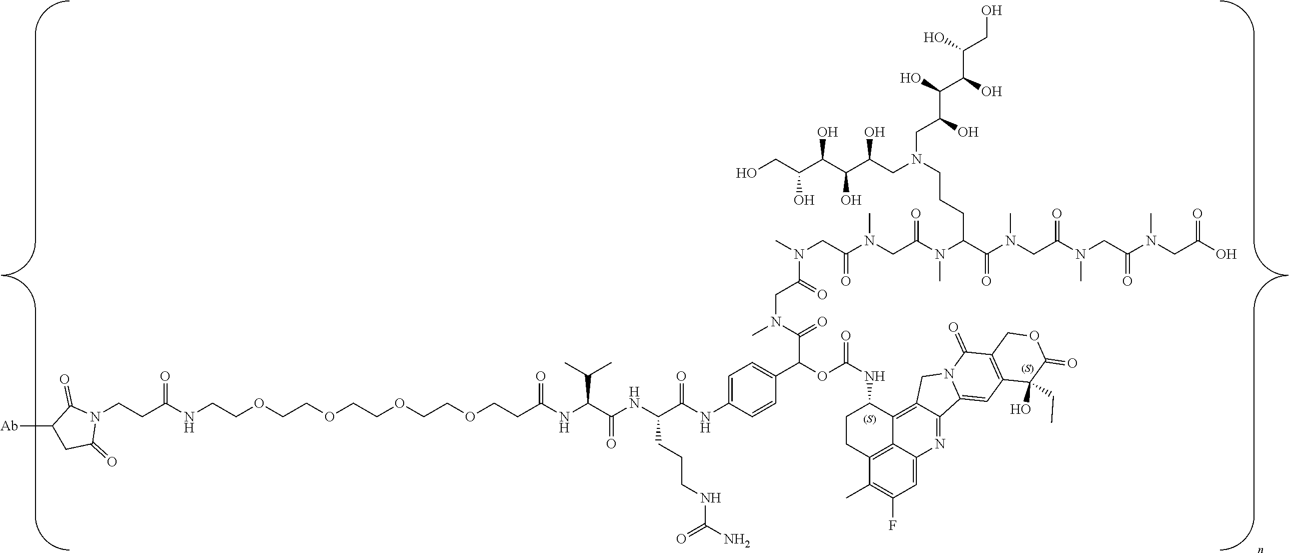

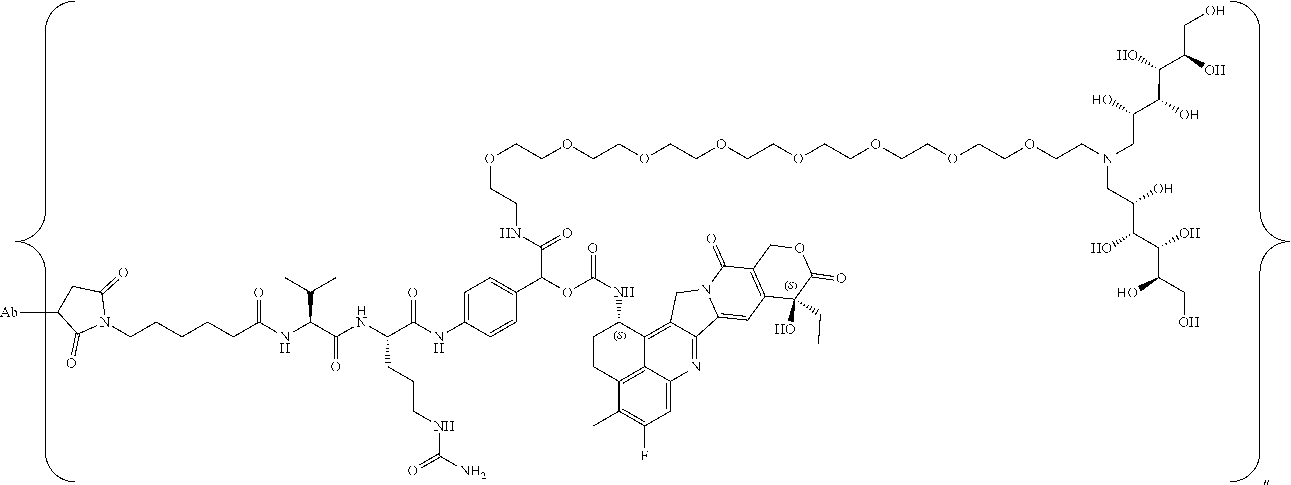

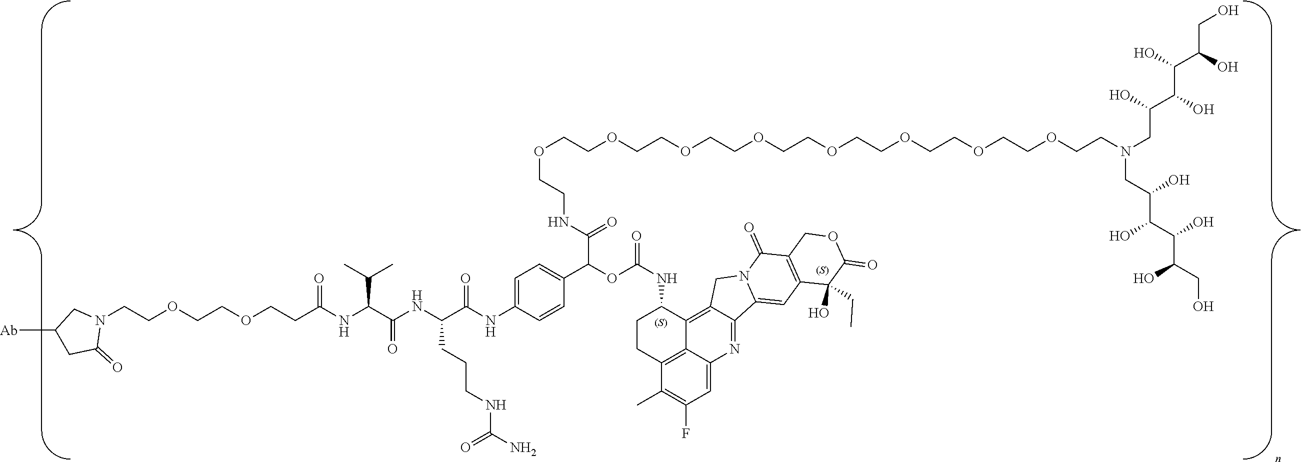

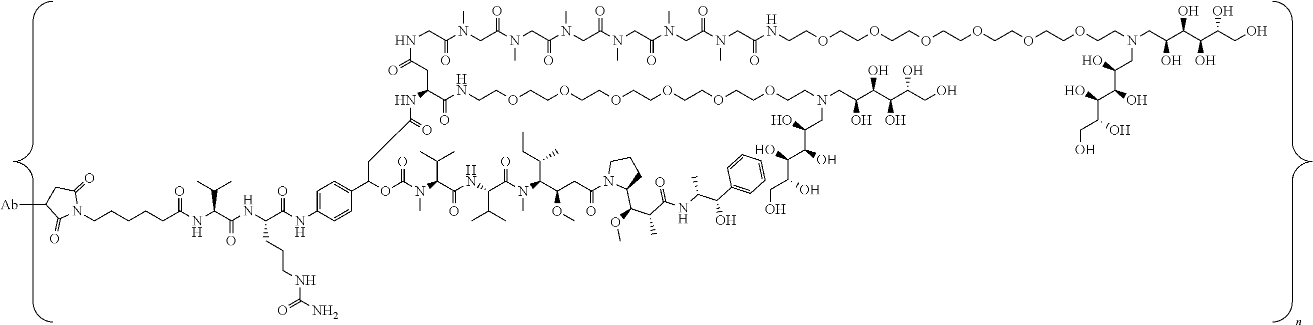

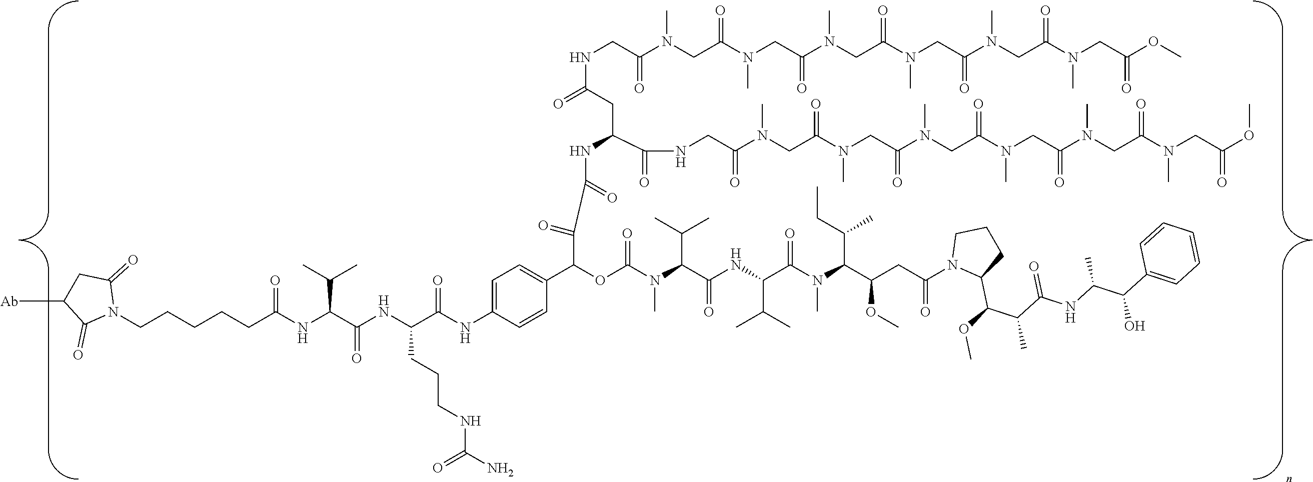

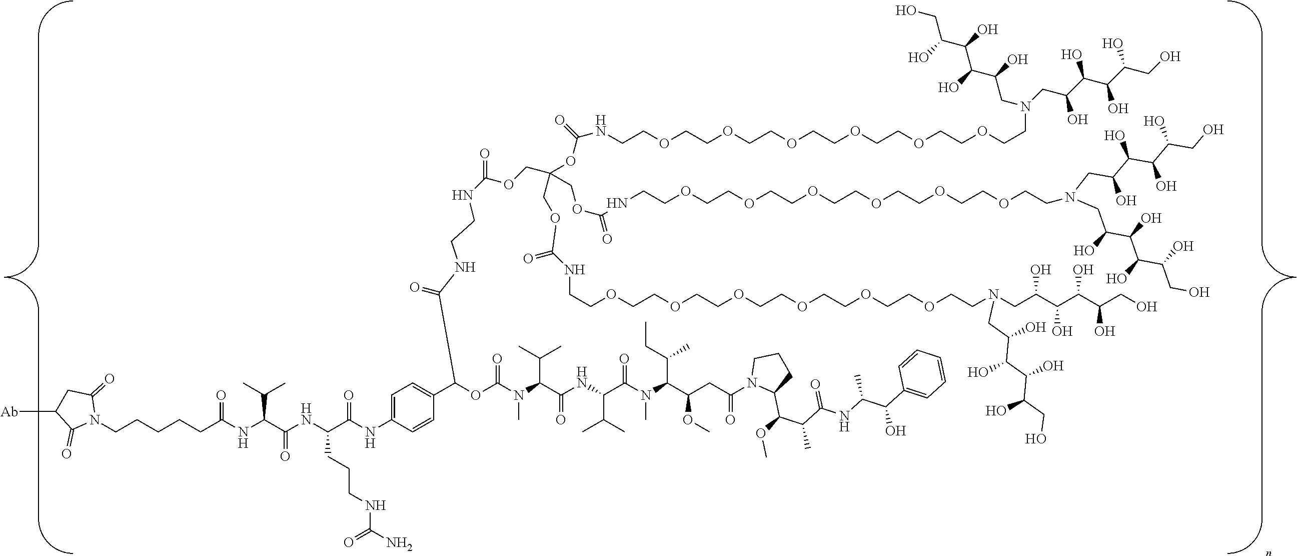

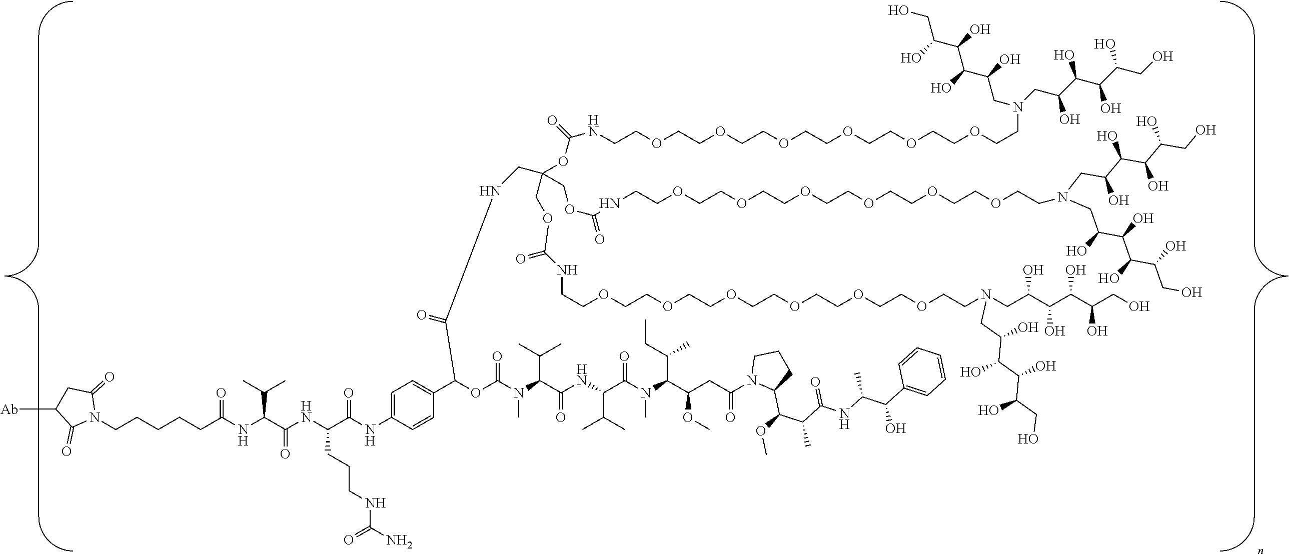

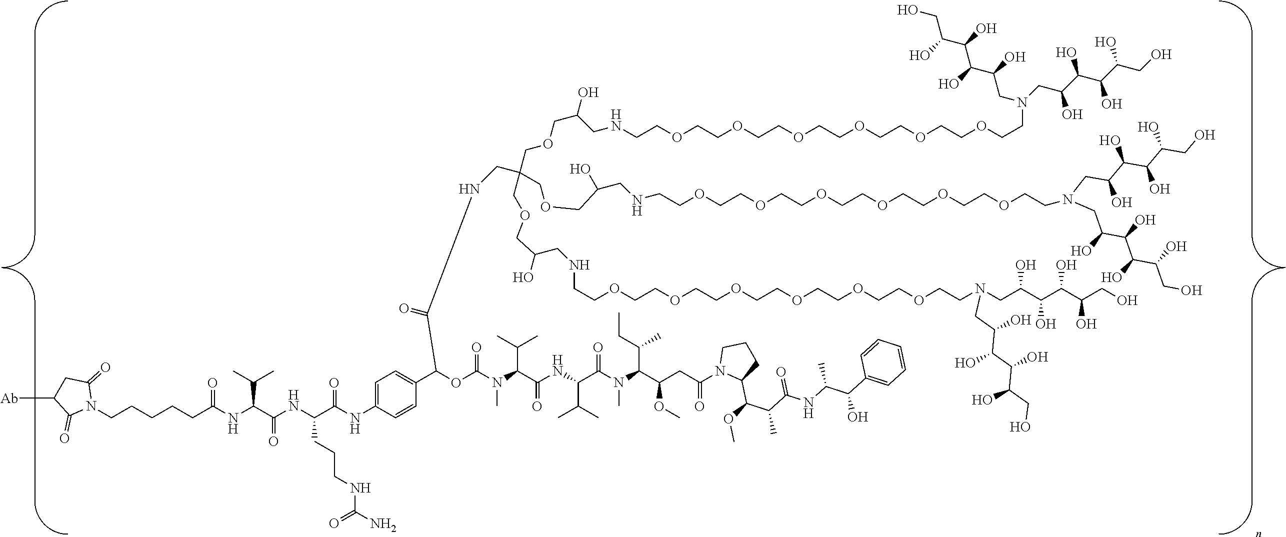

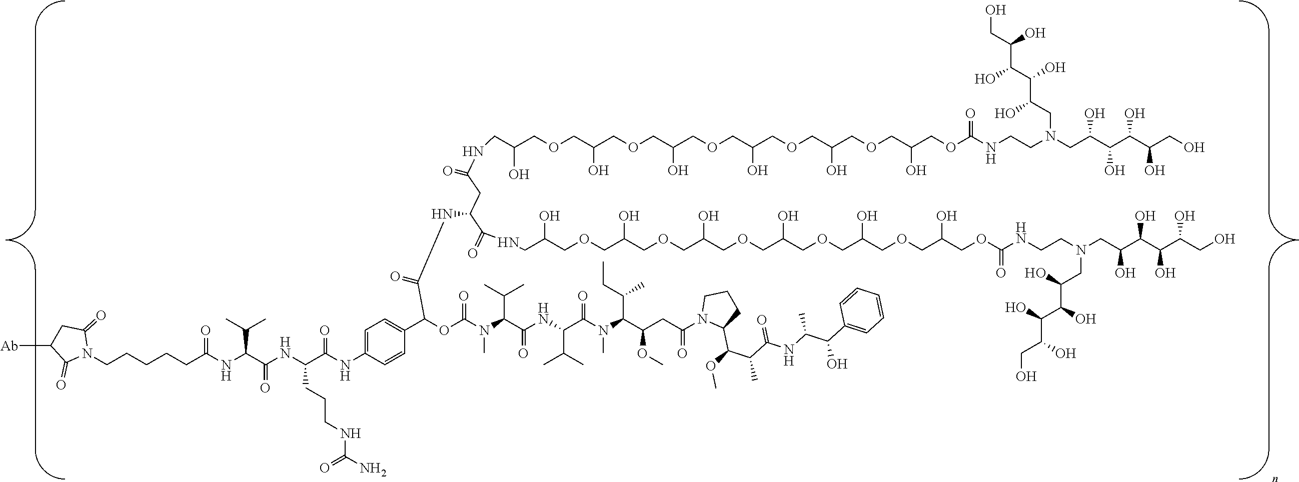

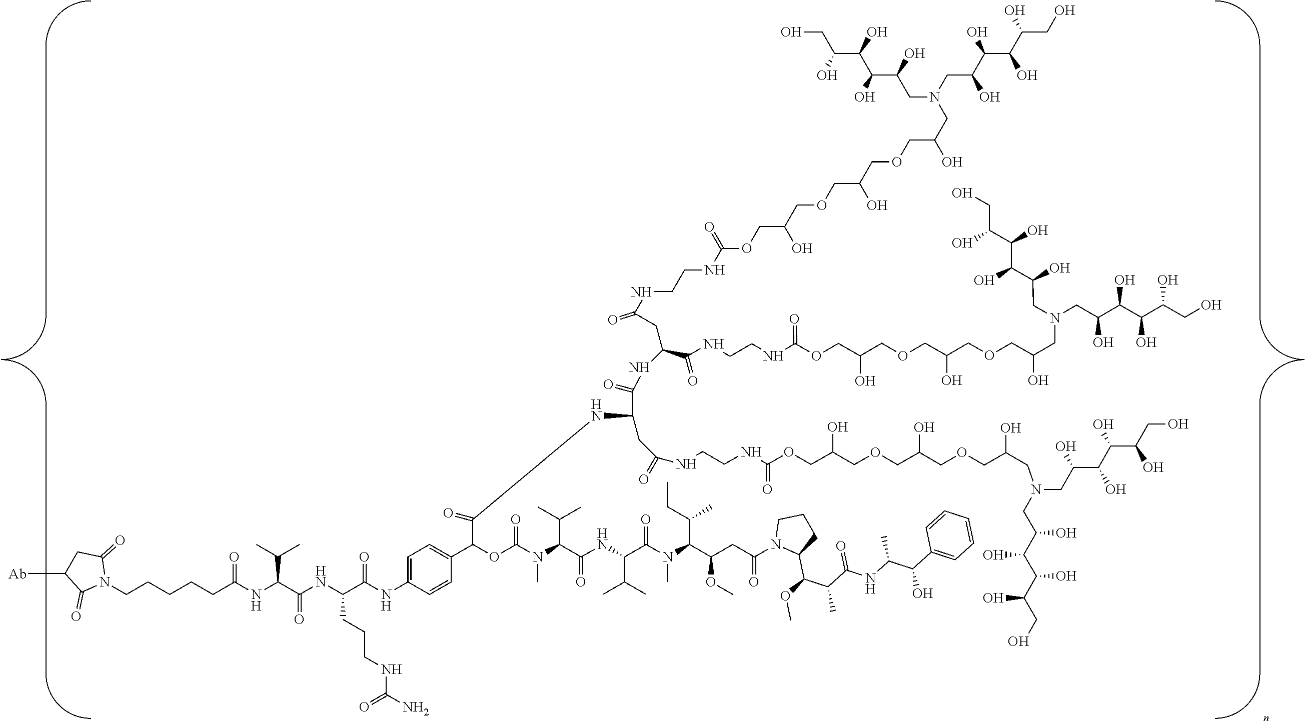

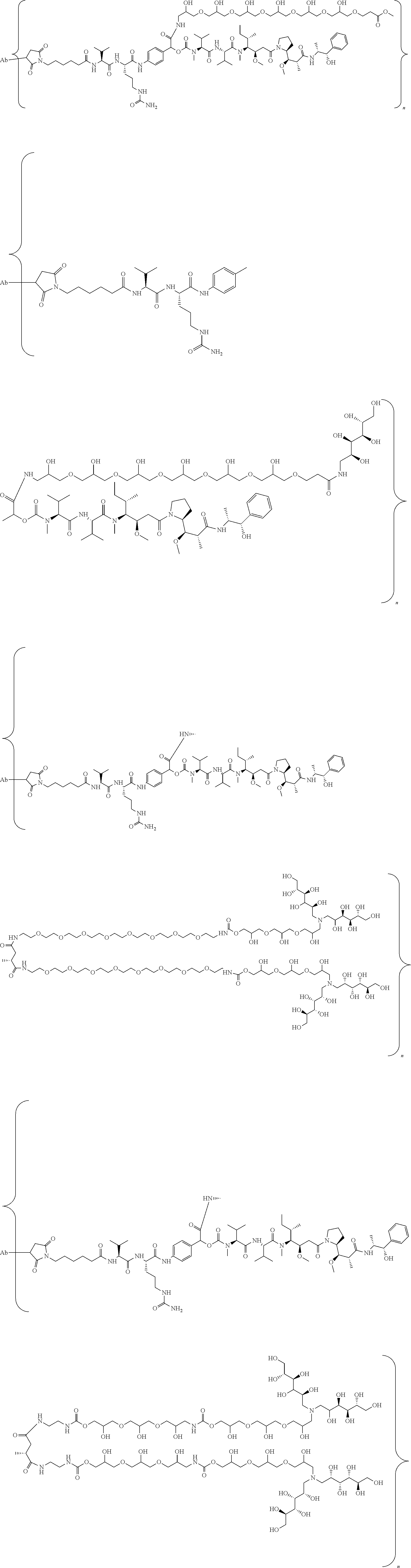

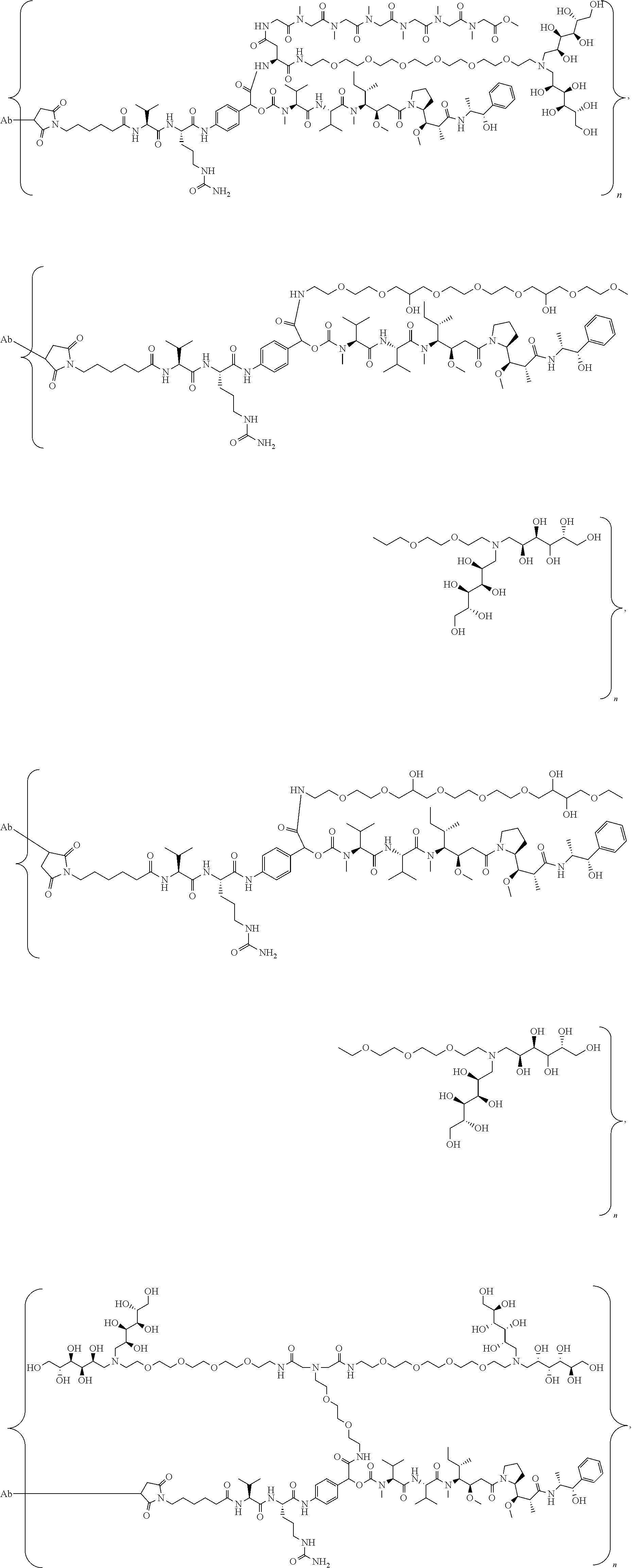

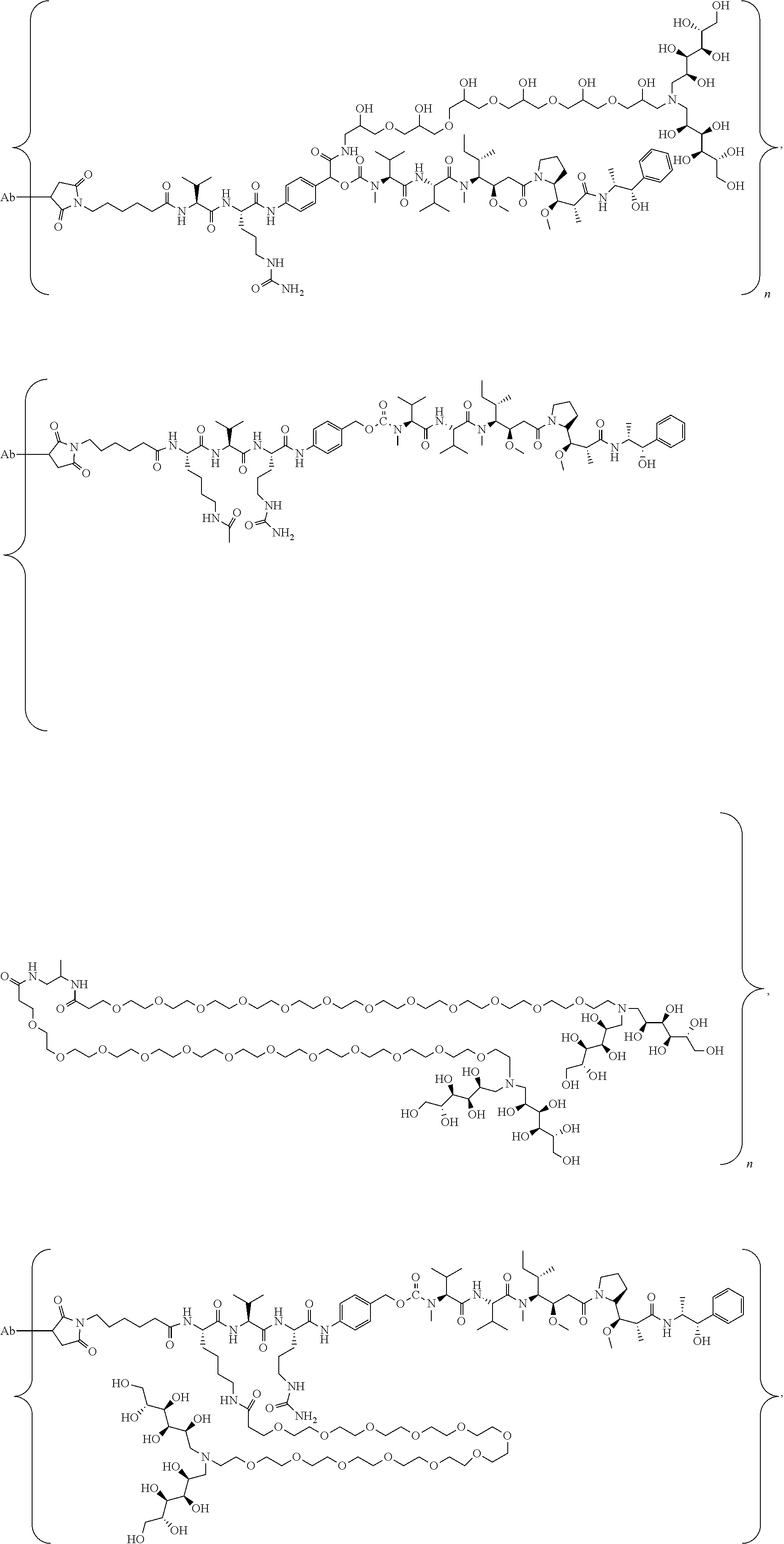

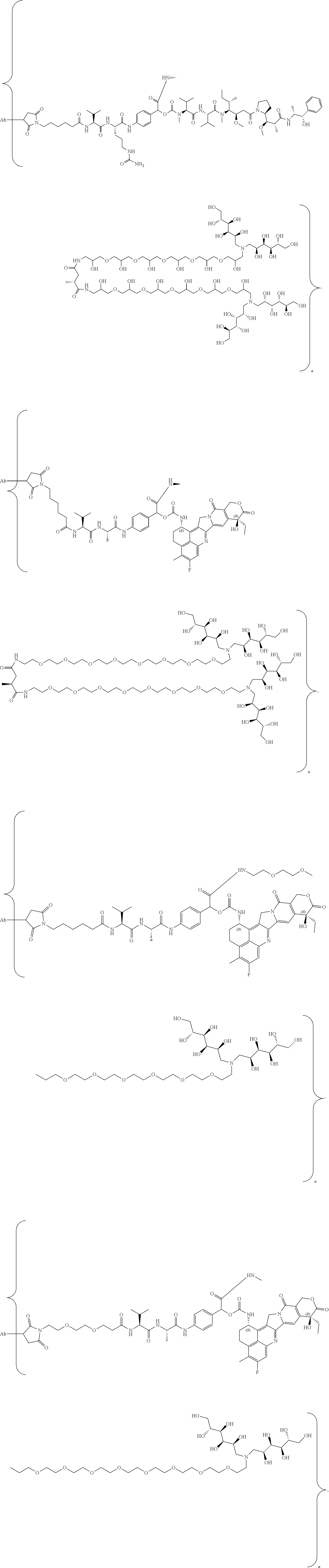

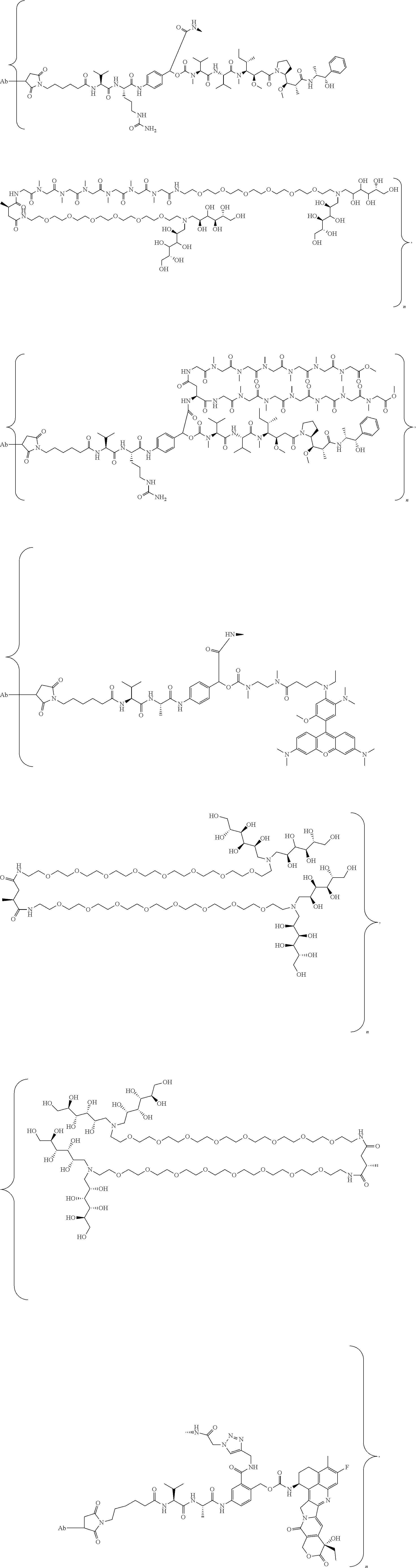

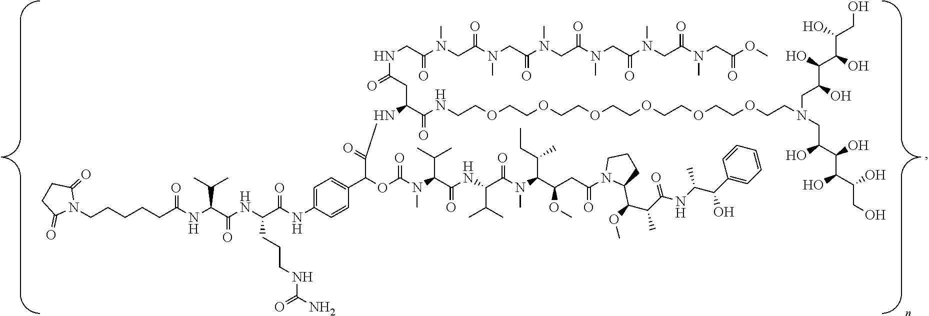

or a stereoisomer thereof, wherein Ab is a EGFR and c-MET binding agent and n is p^{load}.

Embodiment 33. The conjugate of Embodiment 30 or 31, selected from the following:

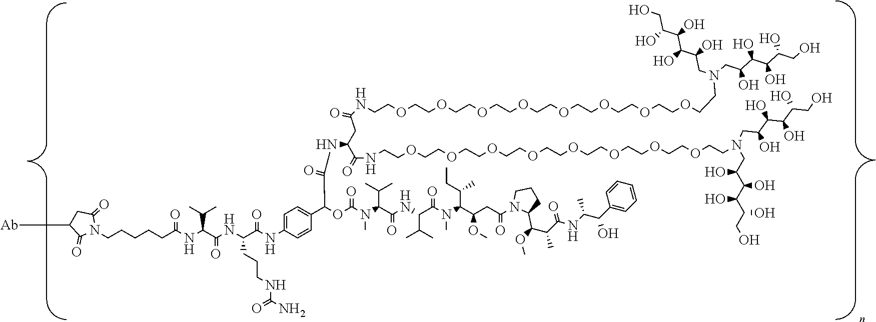

or a stereoisomer thereof, wherein Ab is the binding agent and n is p^{load}.

Embodiment 34. The conjugate of Embodiment 30 or 31, selected from the following:

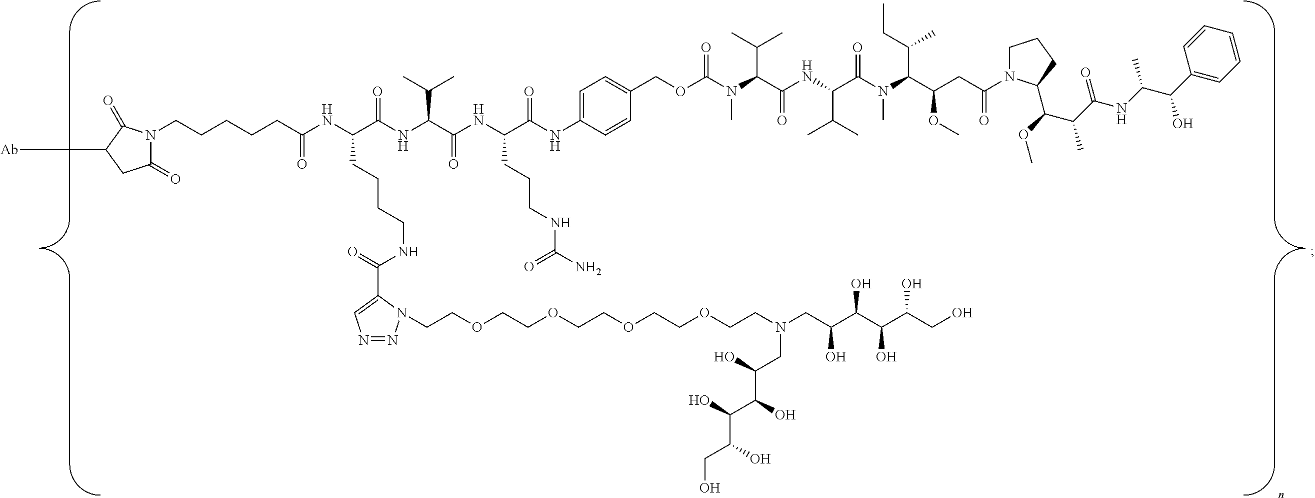

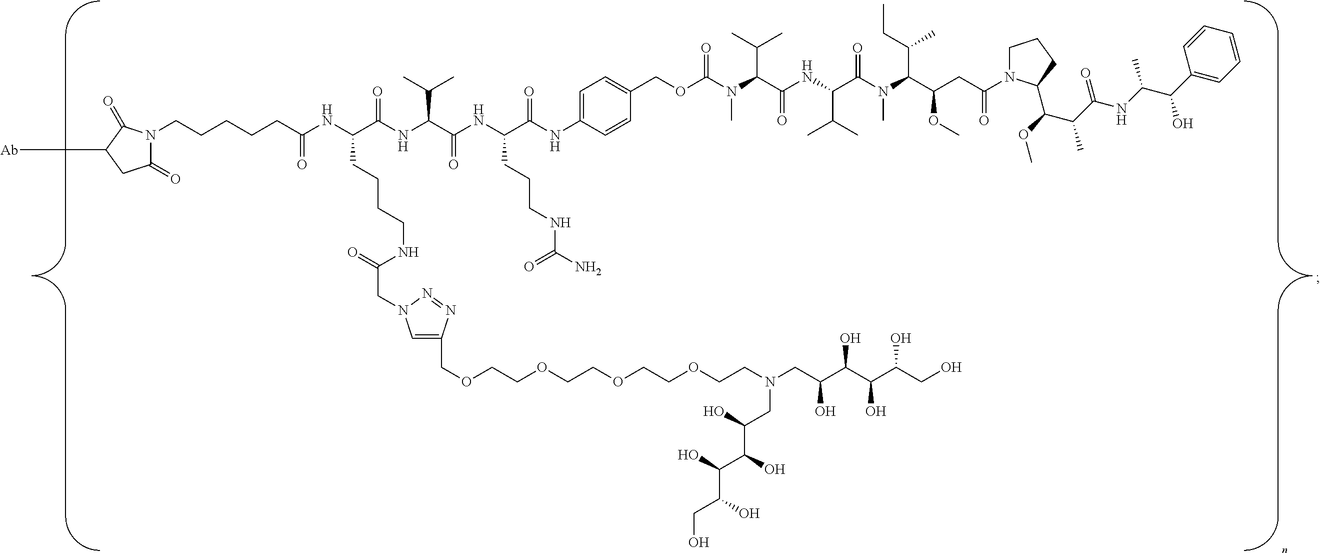

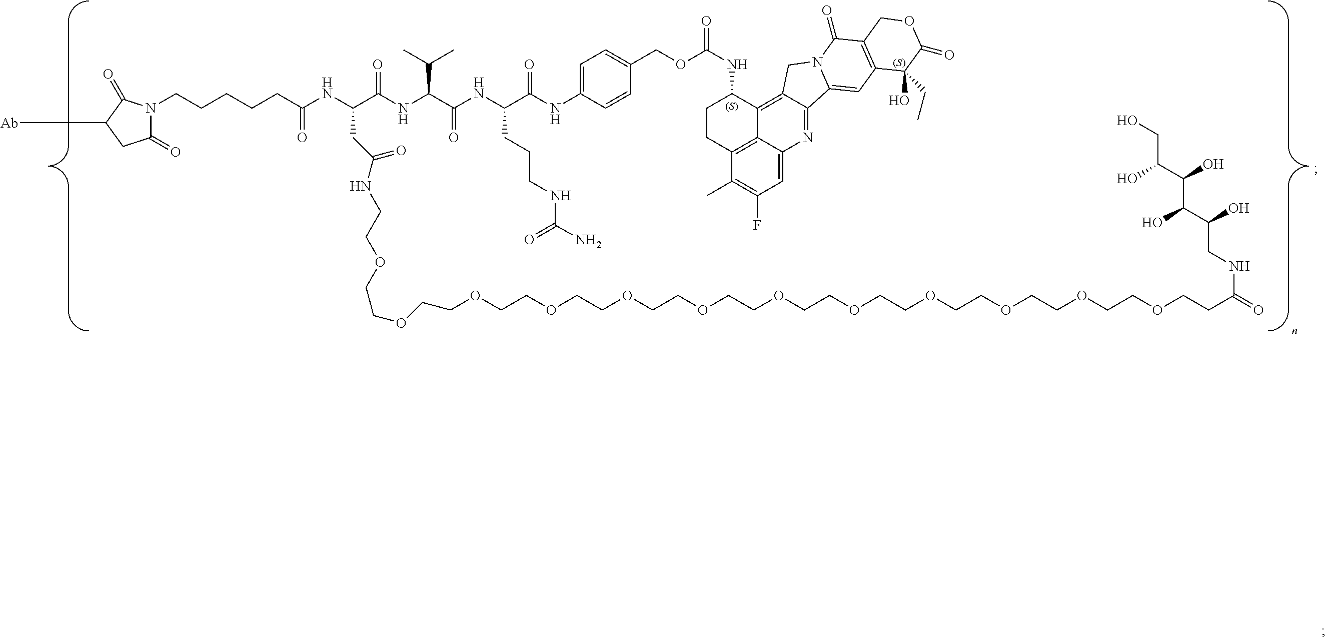

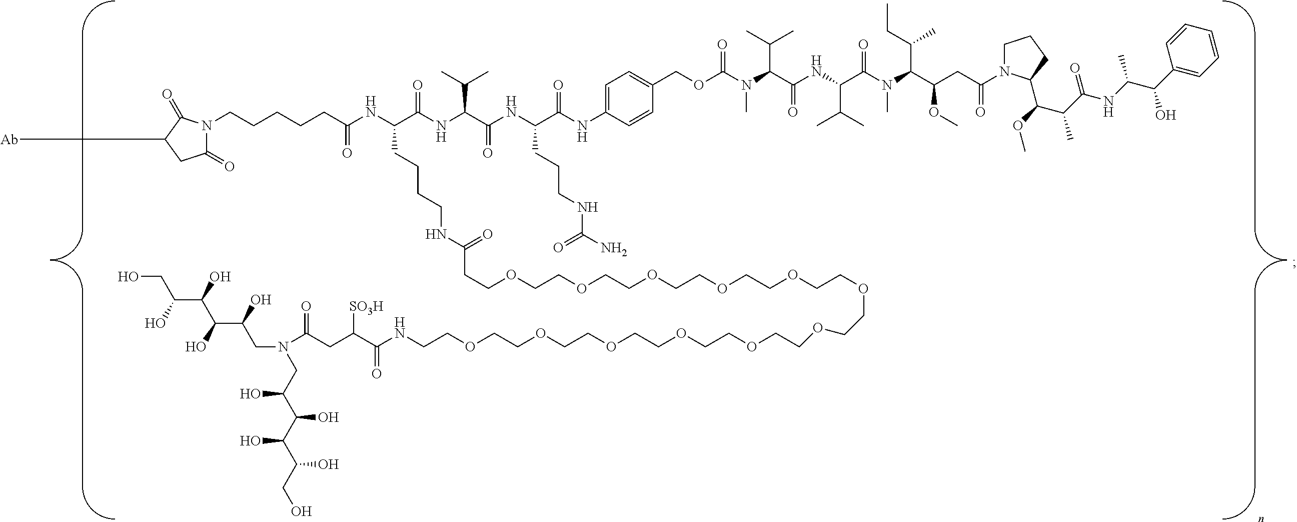

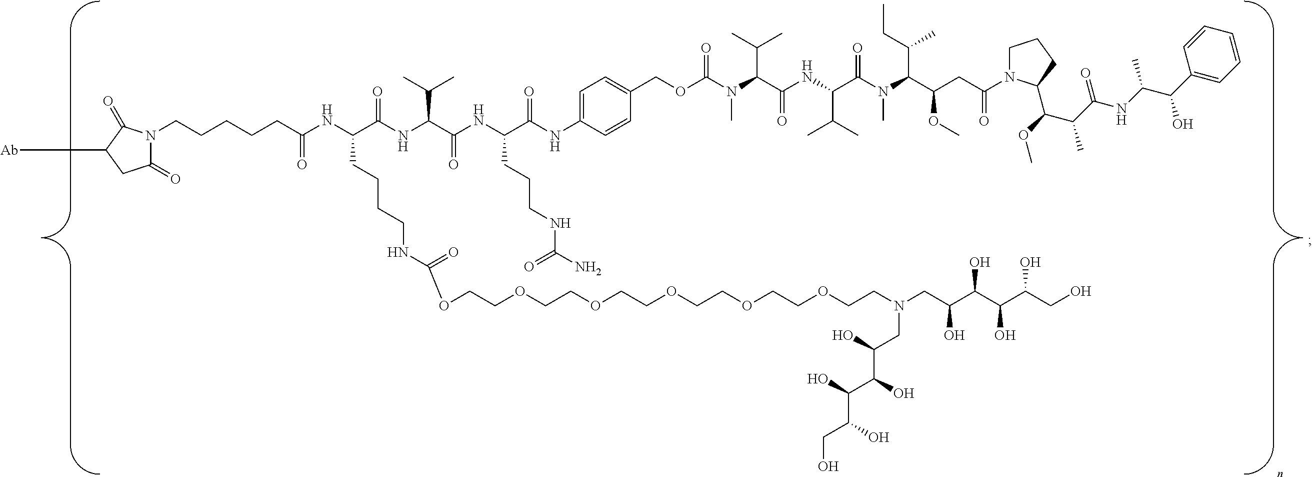

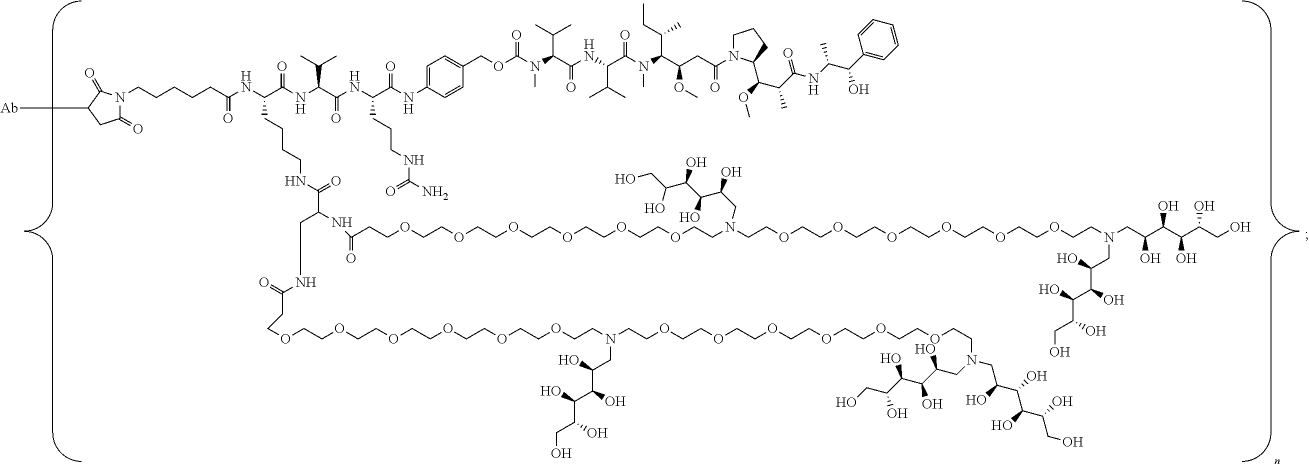

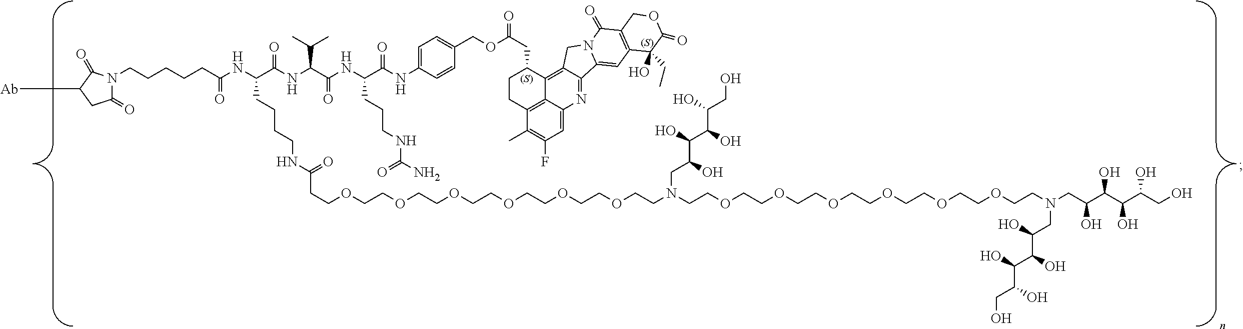

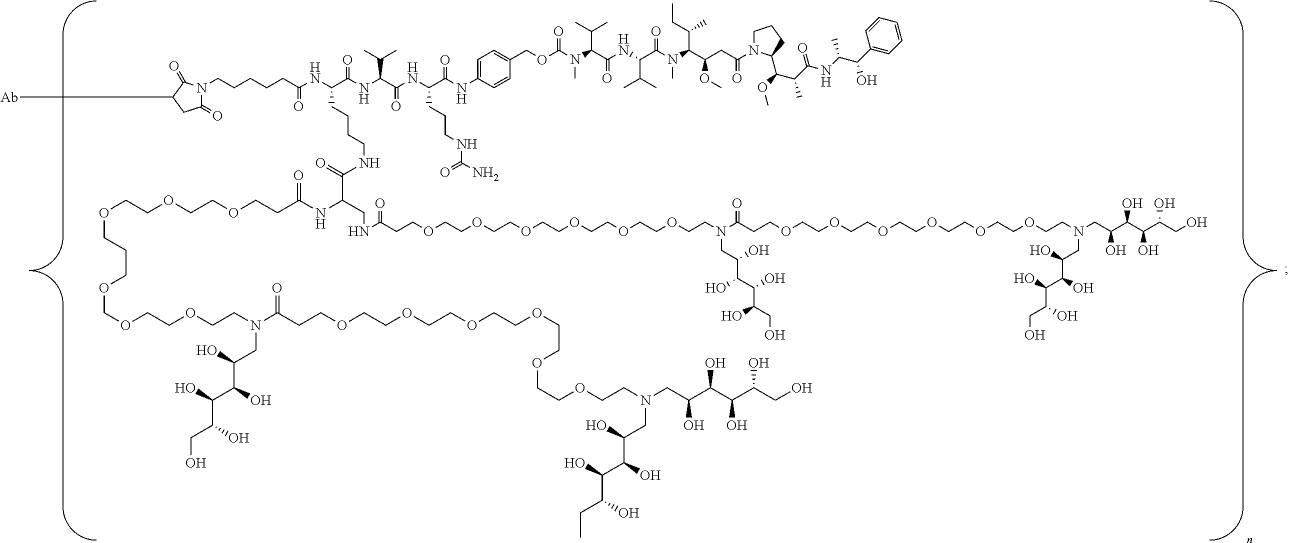

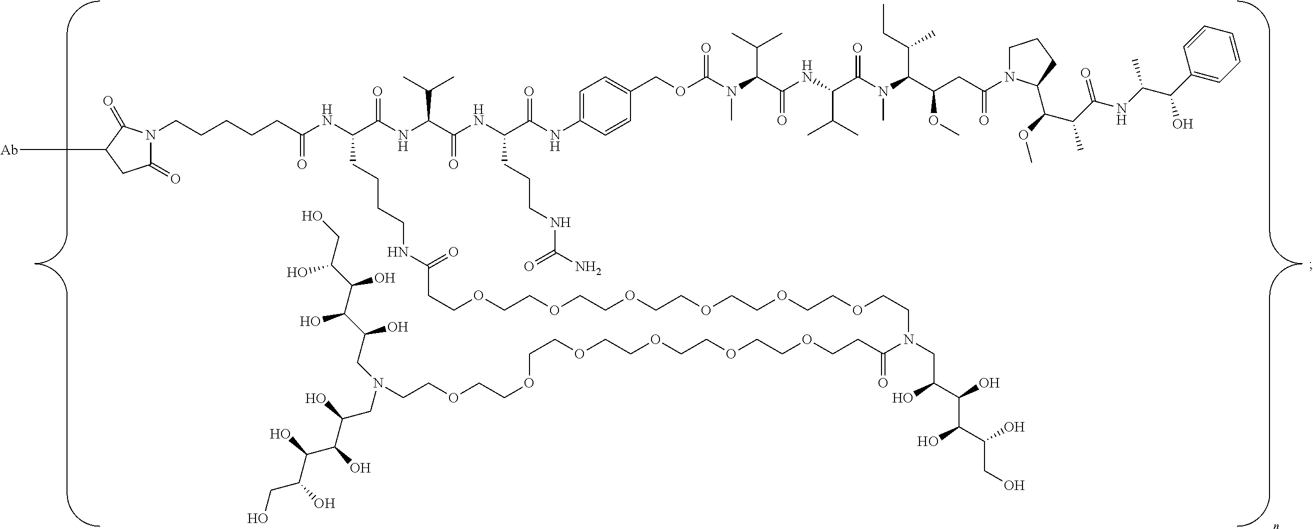

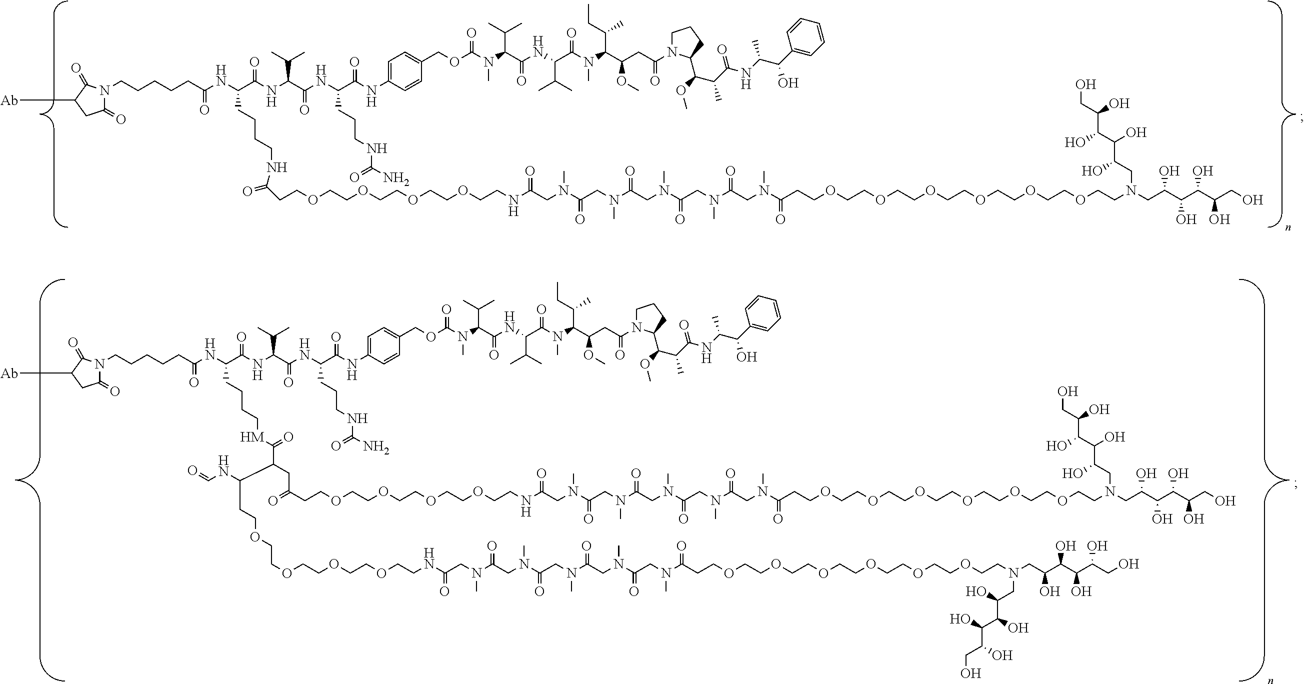

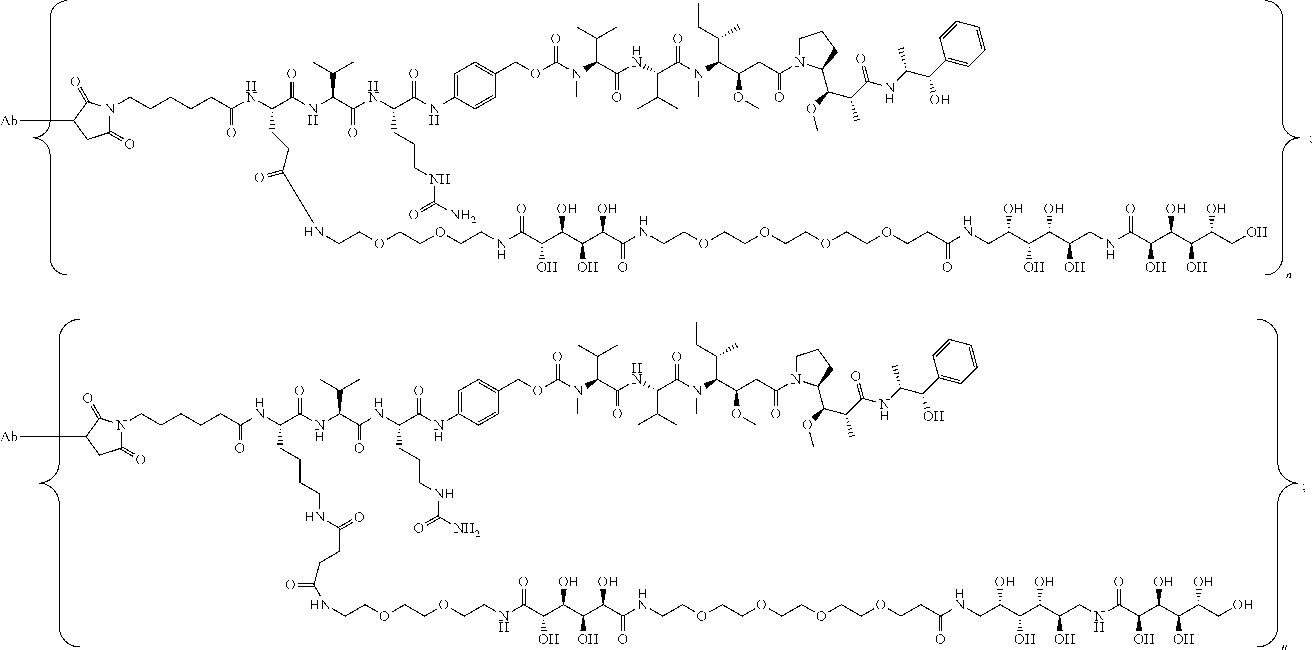

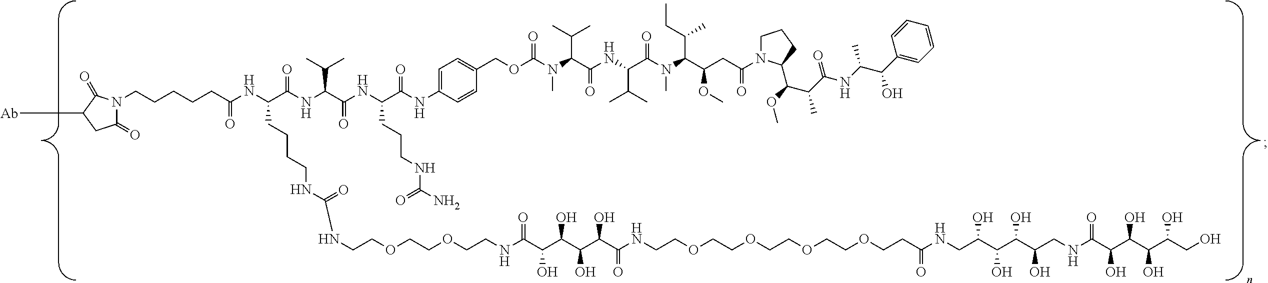

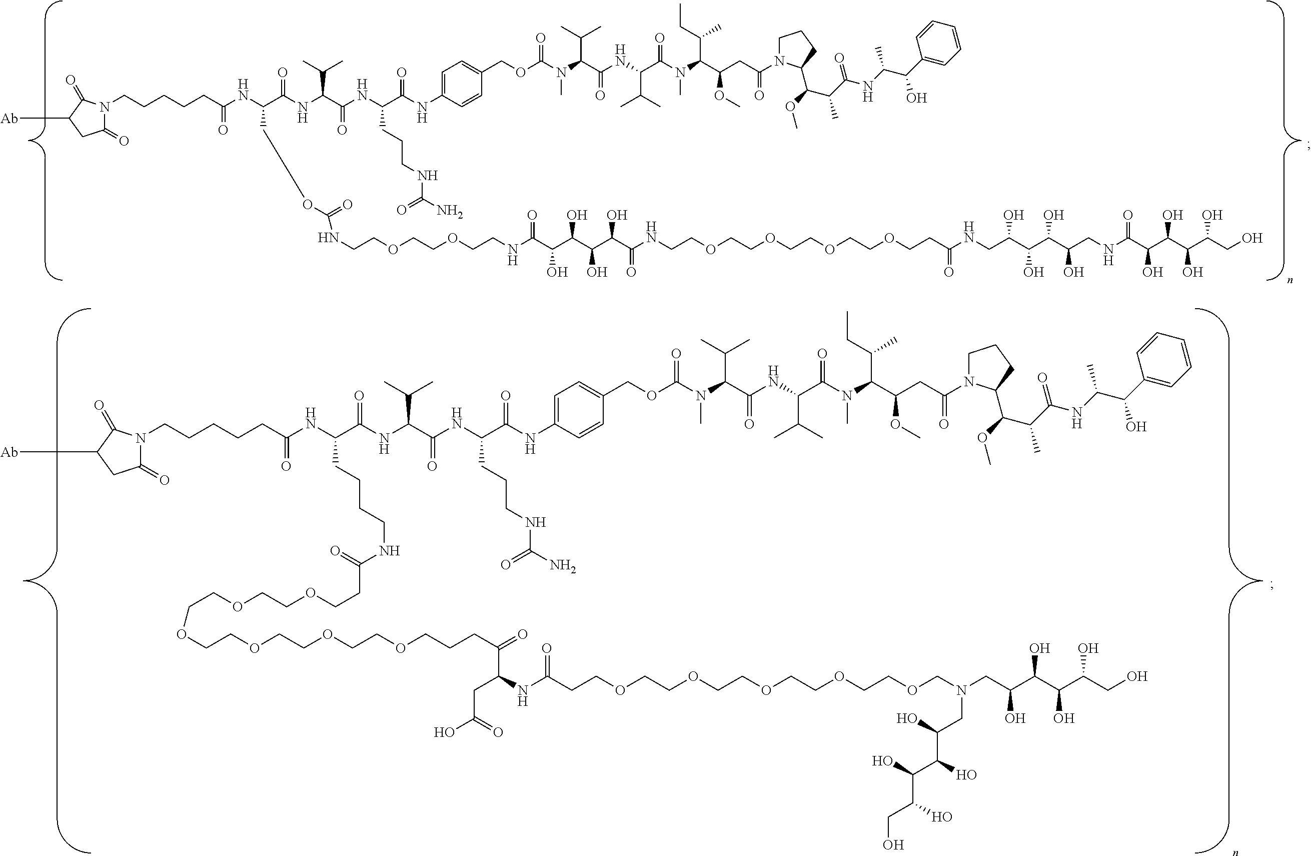

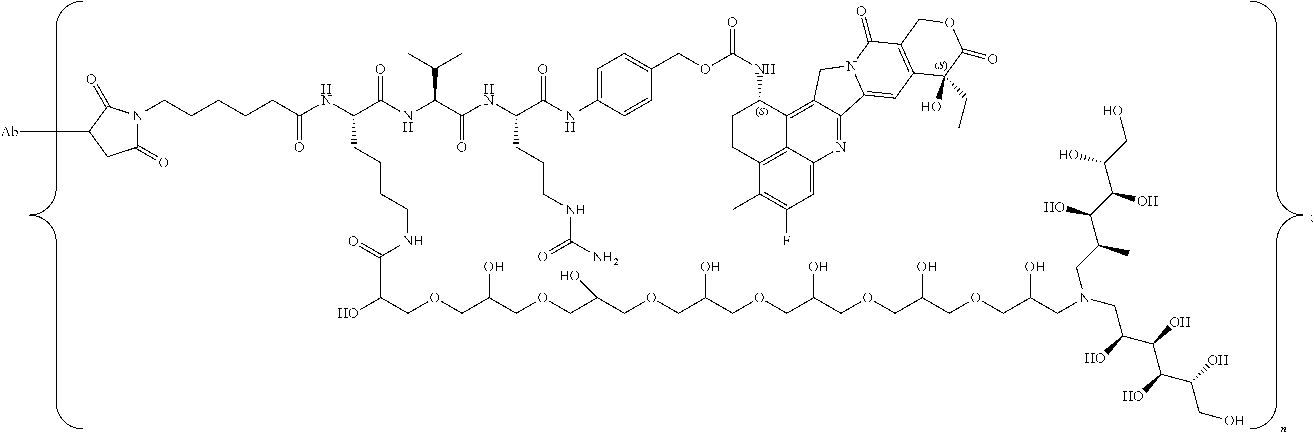

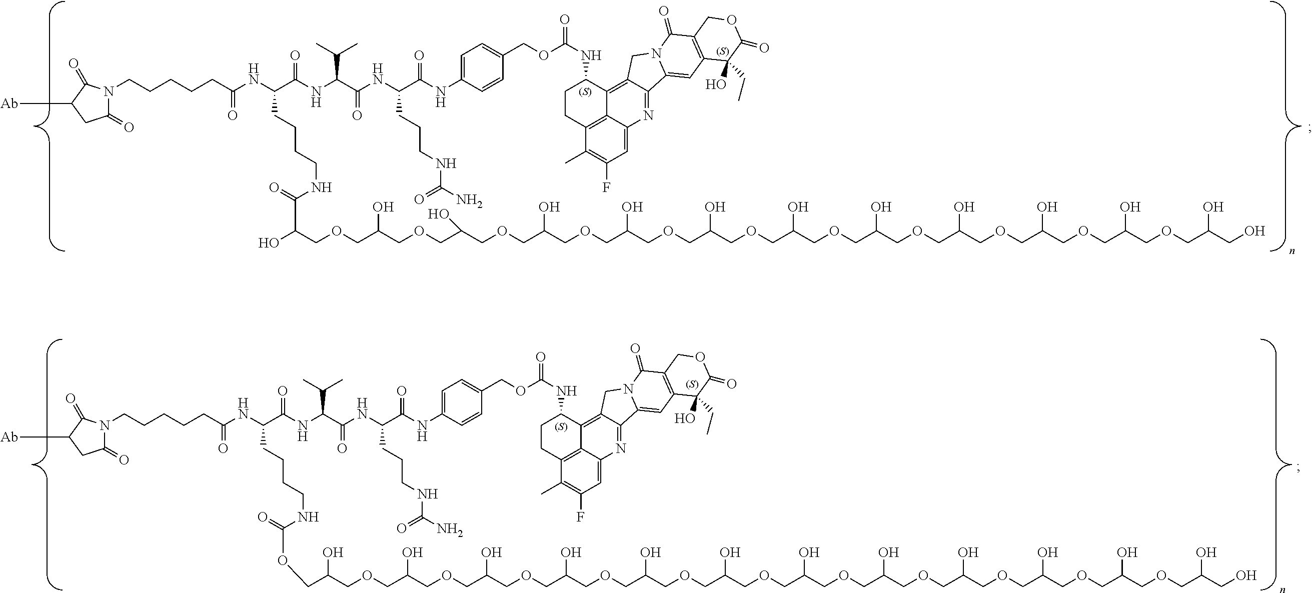

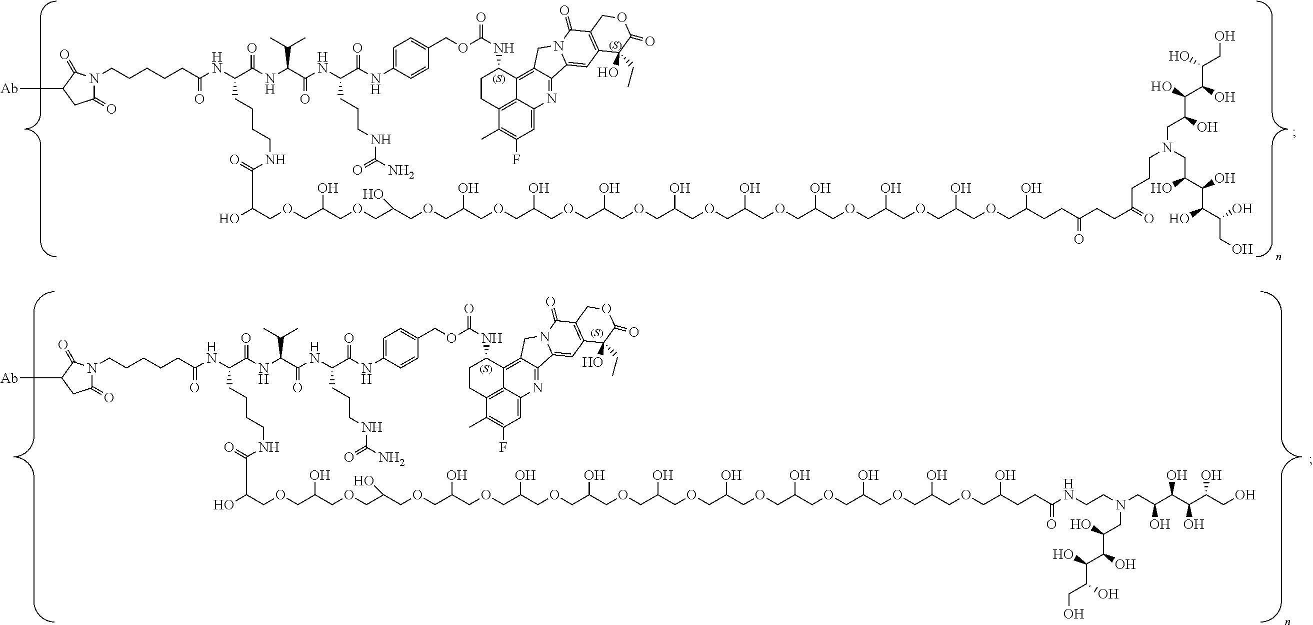

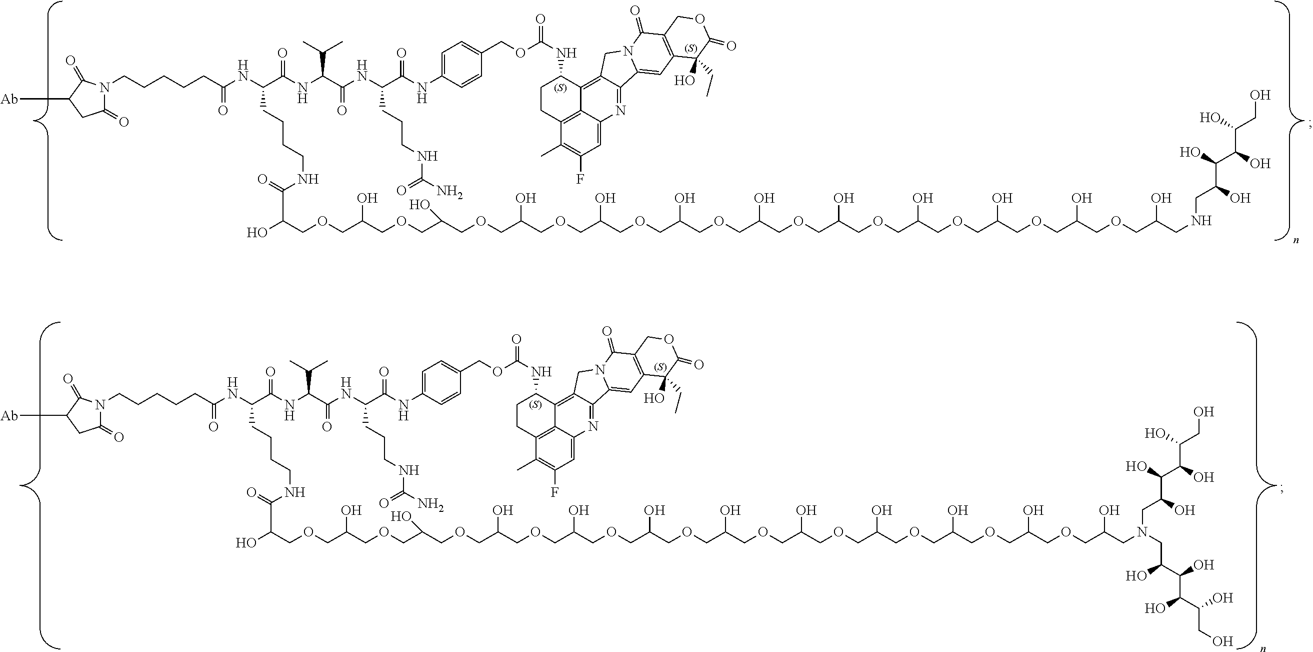

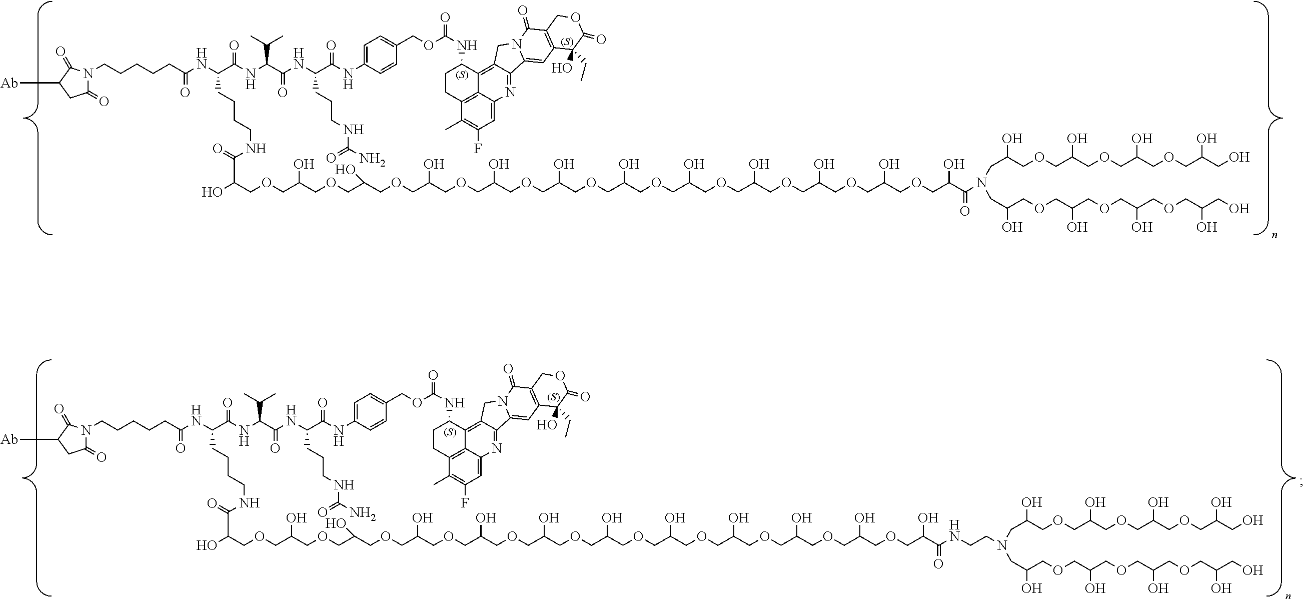

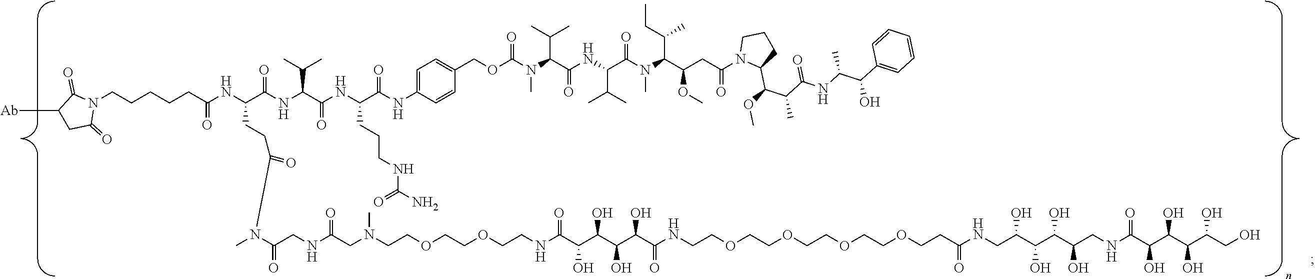

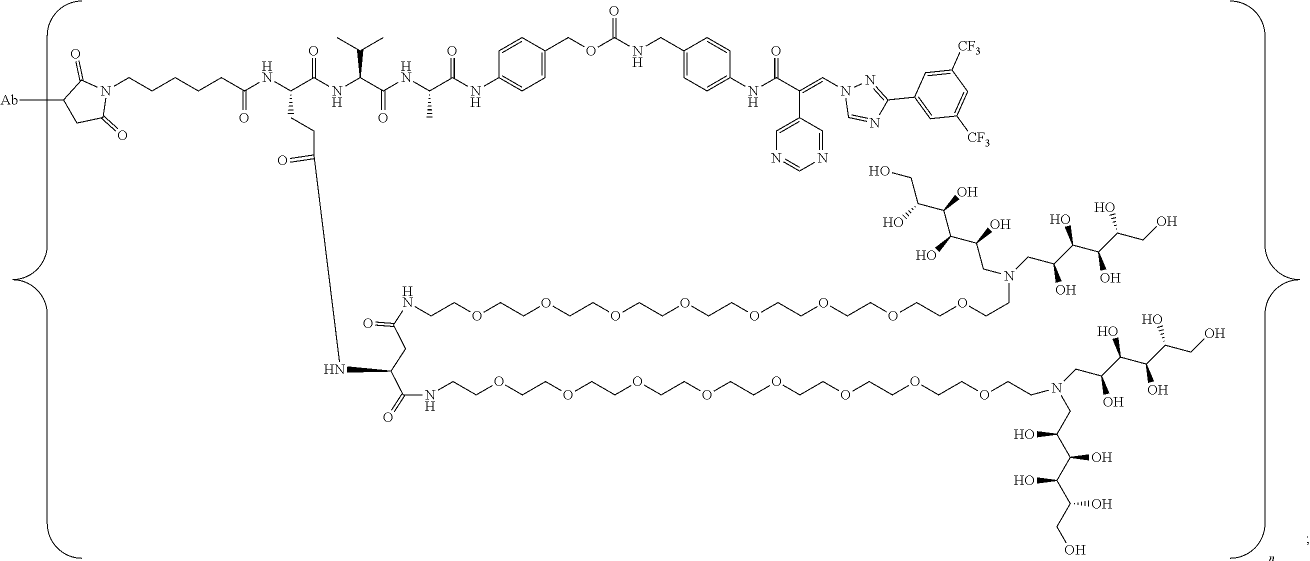

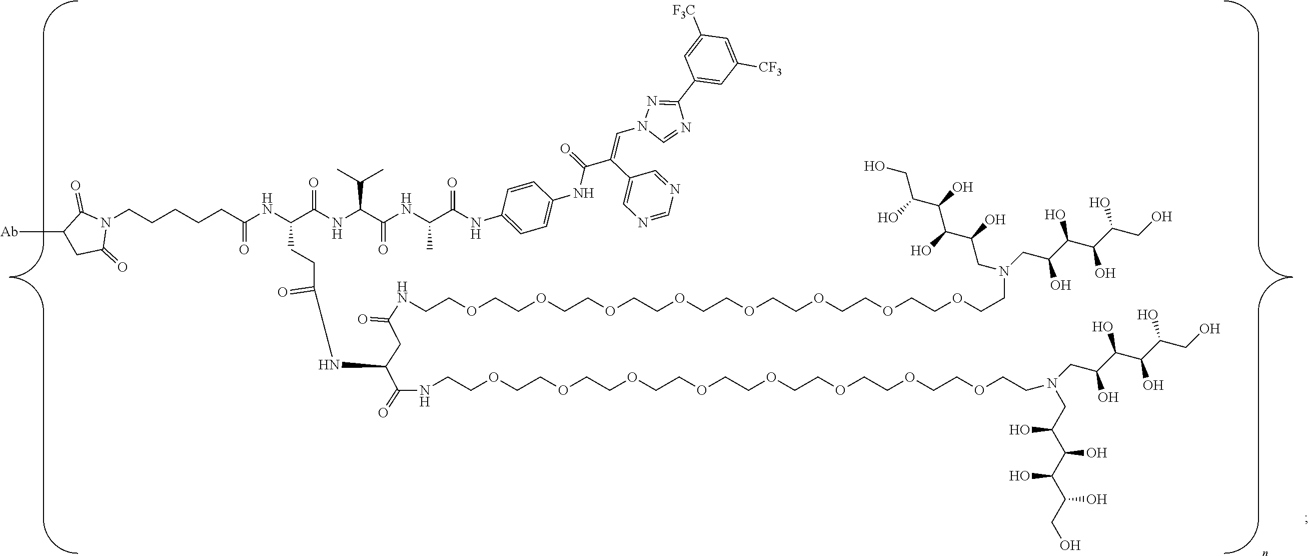

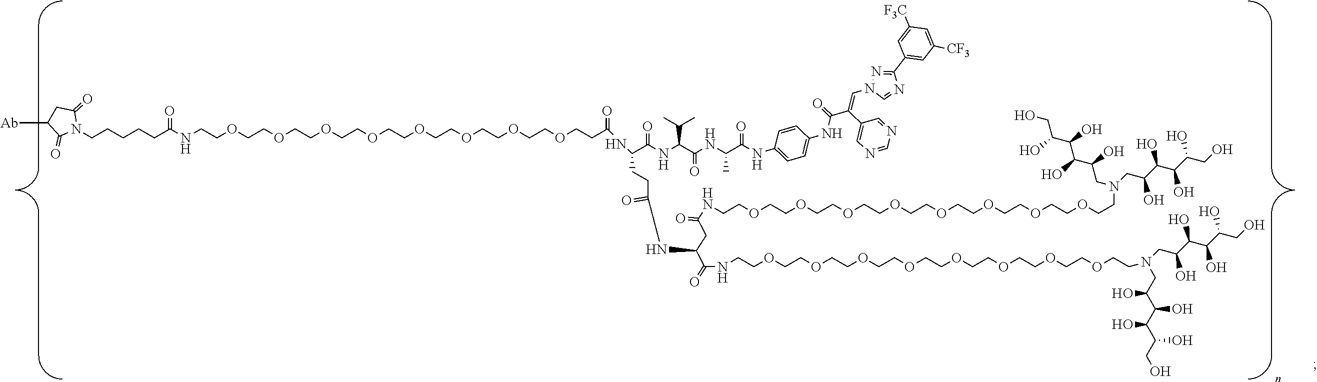

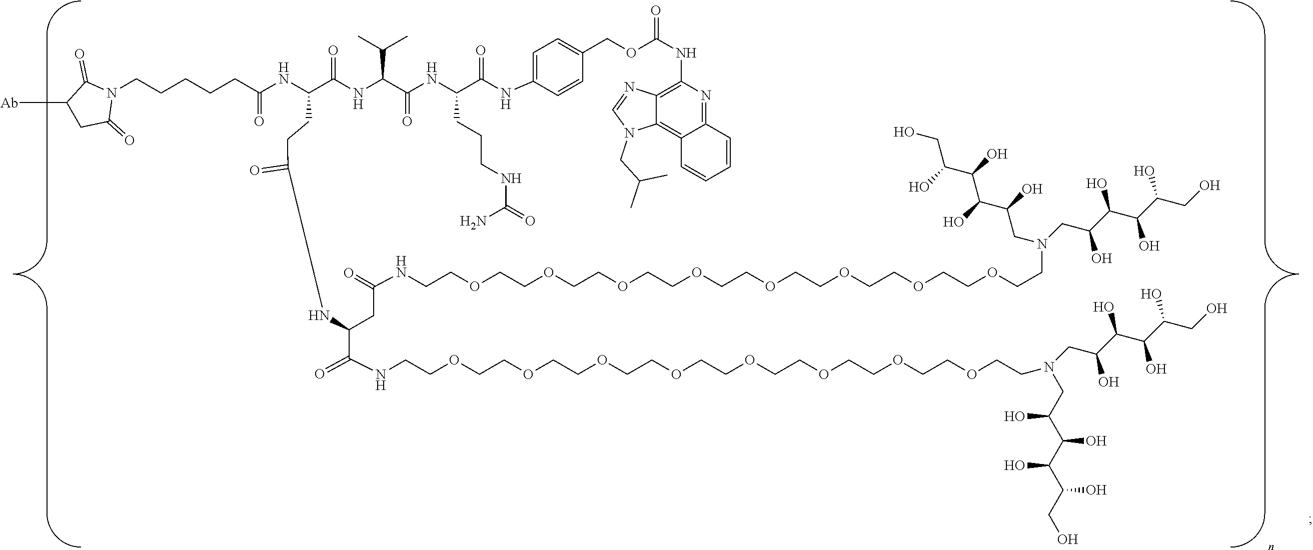

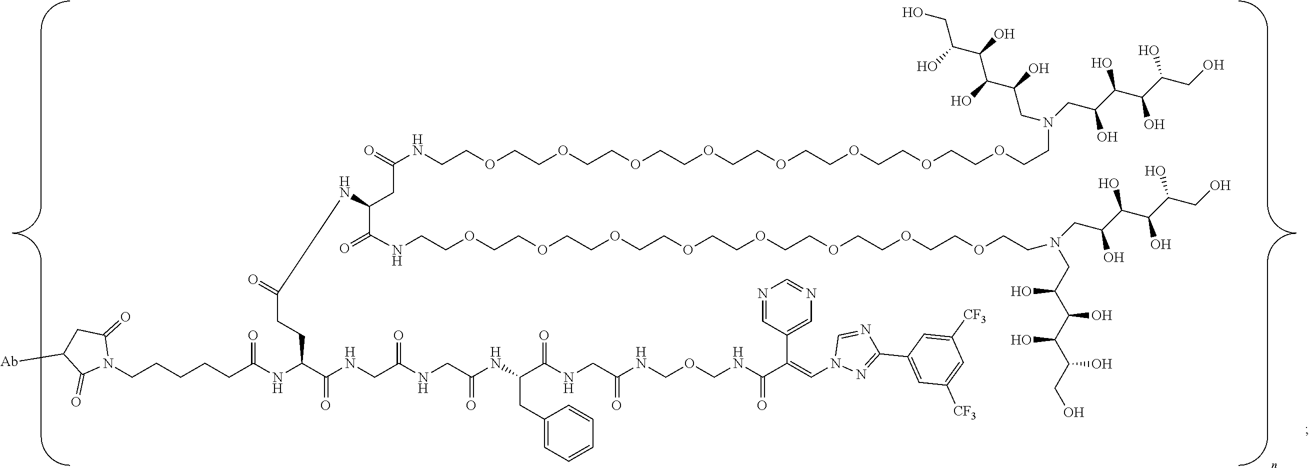

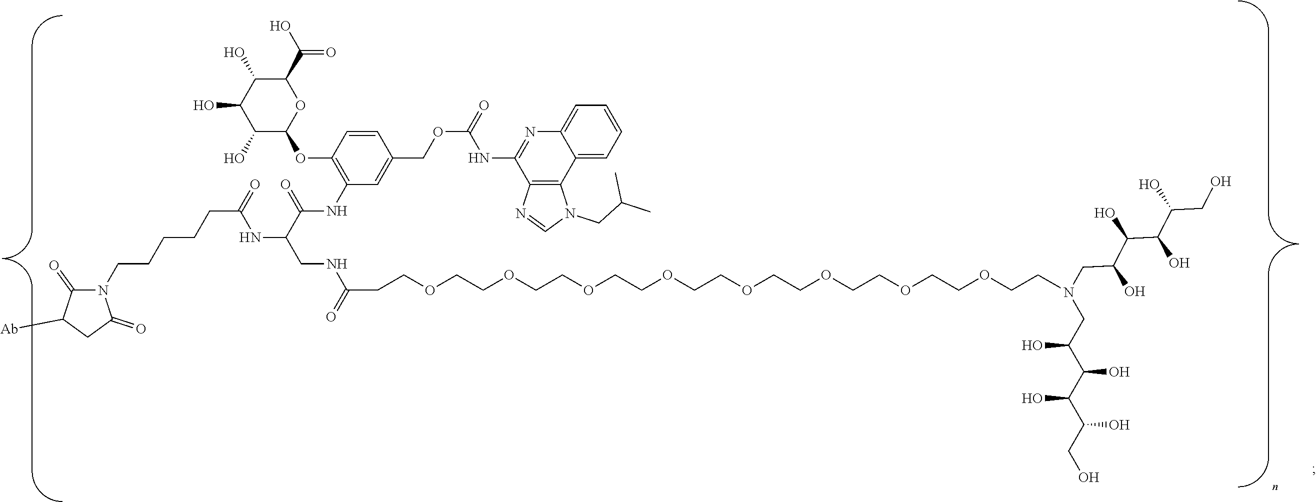

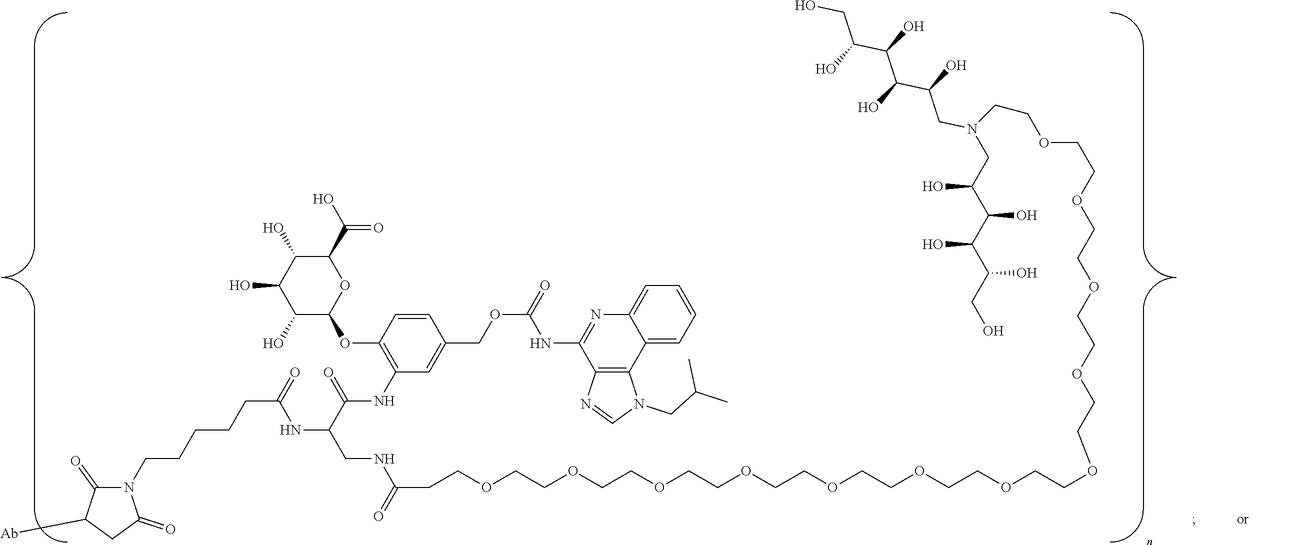

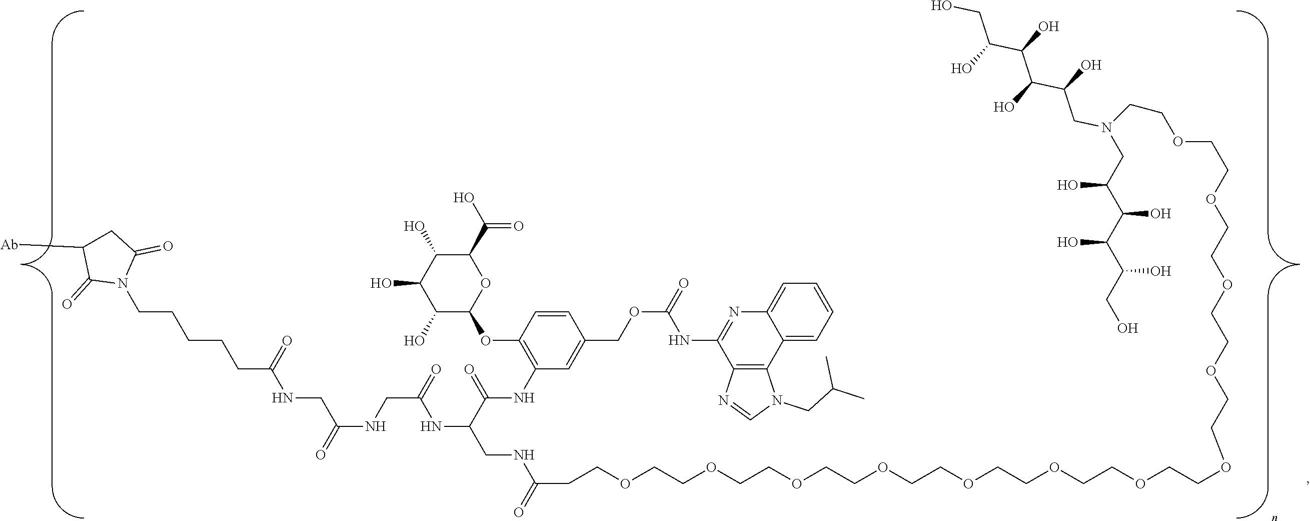

or a stereoisomer thereof, wherein Ab is a EGFR and c-MET binding agent and n is p^{load}.

Embodiment 35. The conjugate of Embodiment 30 or 31, selected from the following:

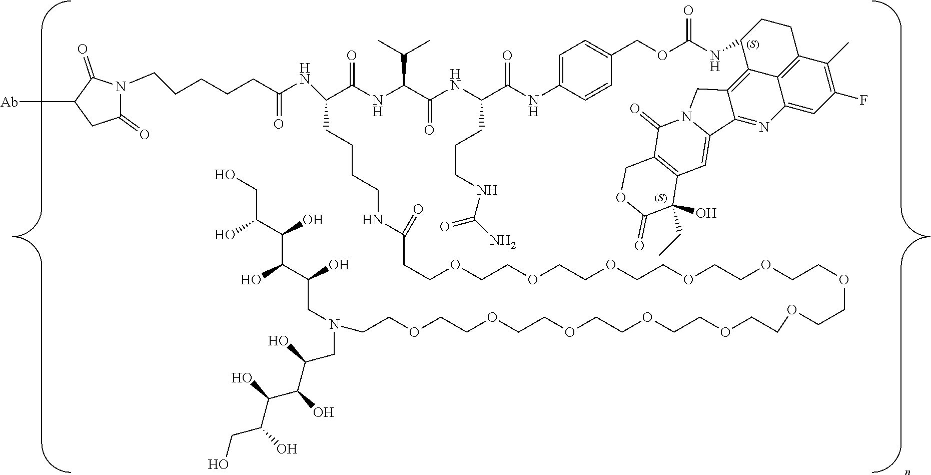

or a stereoisomer thereof, wherein Ab is the binding agent and n is p^{load}.

More descriptions regarding the linker, the linker compound, the drug-linker compound, and the conjugate may be found in, for example, U.S. Application No. 63/619,728, filed on Jan. 10, 2024, International Application No. PCT/US24/11307, filed on Jan. 11, 2024, and International Application No. PCT/CN2024/071901, filed on Jan. 11, 2024, the contents of each of which are hereby incorporated by reference.

### Drug Loading

Conjugates can contain one or more drug unit per EGFR and c-MET bispecific binding agent. The number of drug units per EGFR and c-MET bispecific binding agent is referred to as drug loading. The drug loading of a Conjugate is represented by p^{load} the average number of drug units (drug molecules (e.g., cytotoxic agents)) per EGFR and c-MET bispecific binding agent (e.g., an antibody or antigen binding portion or non-antibody scaffold or non-antibody protein) in a conjugate. For example, if p^{load} is about 4, the average drug loading taking into account all of the EGFR and c-MET bispecific binding agent (e.g., antibodies or antigen binding portion or non-antibody scaffold or non-antibody proteins) present in the composition is about 4. In some embodiments, p^{load} ranges from about 3 to about 5, from about 3.6 to about 4.4, or from about 3.8 to about 4.2. In some embodiments, p^{load} can be about 3, about 4, or about 5. In some embodiments, p^{load} ranges from about 6 to about 8, more preferably from about 7.5 to about 8.4. In some embodiments, p^{load} can be about 6, about 7, or about 8. In some embodiments, p^{load} ranges from about 8 to about 16.

The average number of drug units per EGFR and c-MET bispecific binding agent (e.g., antibody or antigen binding portion or non-antibody scaffold) in a preparation may be characterized by conventional means such as UV, mass spectroscopy, Capillary Electrophoresis (CE), and HPLC. The quantitative distribution of conjugates in terms of p^{load} may also be determined. In some instances, separation, purification, and characterization of homogeneous conjugates where p^{load} is a certain value from conjugates with other drug loadings may be achieved by means such as reverse phase HPLC or Hydrophobic Interaction Chromatography (HIC) HPLC.

### Attachment of Drug-Linkers to Antibodies, Antigen Binding Portions and Other Binding Agents (Including Non-Antibody Scaffolds)

Techniques for attaching drug unit(s) to EGFR and c-MET bispecific binding agent (such as antibodies or antigen binding portions thereof or non-antibody scaffolds) via linkers are well-known in the art. See, e.g., Alley et al., Current Opinion in Chemical Biology 2010 14:1-9; Senter, Cancer J., 2008, 14(3):154-169. In some embodiments, a linker is first attached to a drug unit (e.g., a cytotoxic agent(s), immune modulatory agent or other agent) and then the drug-linker(s) is attached to the EGFR and c-MET bispecific binding agent (e.g., an antibody or antigen binding portion thereof or non-antibody protein scaffold). In some embodiments, a linker(s) is first attached to an EGFR and c-MET bispecific binding agent (e.g., an antibody or antigen binding portion thereof or non-antibody protein scaffold), and then a drug unit is attached to a linker. In the following discussion, the term drug-linker is used to exemplify attachment of linkers or drug-linkers to the EGFR and c-MET bispecific binding agent; the skilled artisan will appreciate that the selected attachment method can be determined according to linker and the drug unit. In some embodiments, a drug unit is attached to an EGFR and c-MET bispecific binding agent via a linker in a manner that reduces the activity of the drug unit until it is released from the conjugate (e.g., by hydrolysis, by proteolytic degradation or by a cleaving agent.).

Generally, a conjugate may be prepared by several routes employing organic chemistry reactions, conditions, and reagents known to those skilled in the art, including: (1) reaction of a nucleophilic group of an EGFR and c-MET bispecific binding agent (e.g., an antibody or antigen binding portion thereof or non-antibody protein scaffold) with a bivalent linker to form a targeting group-linker intermediate via a covalent bond, followed by reaction with a drug unit; and (2) reaction of a nucleophilic group of a drug unit with a bivalent linker, to form drug-linker, via a covalent bond, followed by reaction with a nucleophilic group of an EGFR and c-MET bispecific binding agent. Exemplary methods for preparing conjugates via the latter route are described in U.S. Pat. No. 7,498,298, which is expressly incorporated herein by reference.

Nucleophilic groups on the EGFR and c-MET bispecific binding agent such as antibodies, antigen binding portions and other binding agents (including non-antibody scaffolds) include, but are not limited to: (i)N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linkers including: (i) active esters such as NHS esters, HOBt esters, haloformates, and acid halides; (ii) alkyl and benzyl halides such as haloacetamides; and (iii) aldehydes, ketones, carboxyl, and maleimide groups. The EGFR and c-MET bispecific binding agent, such as antibodies (and antigen binding portions and other binding agents (including non-antibody scaffolds)) has reducible interchain disulfides, i.e., cysteine bridges. Antibodies (and antigen binding portions and other binding agents (including non-antibody scaffolds)) may be made reactive for conjugation with linkers by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into the EGFR and c-MET bispecific binding agent such as antibodies (and antigen binding portions and other binding agents (including non-antibody scaffolds)) through modification of lysine residues, e.g., by reacting lysine residues with 2-iminothiolane (Traut's reagent), resulting in conversion of an amine into a thiol. Reactive thiol groups may also be introduced into the EGFR and c-MET bispecific binding agent (such as an antibody and antigen binding portions and other binding agents (including non-antibody scaffolds)) by introducing one, two, three, four, or more cysteine residues (e.g., by preparing antibodies, antigen binding portions and other binding agents (including non-antibody scaffolds) comprising one or more non-native cysteine amino acid residues).

Conjugates may also be produced by reaction between an electrophilic group on the EGFR and c-MET bispecific binding agent, such as an aldehyde or ketone carbonyl group, with a nucleophilic group on a linker reagent. Useful nucleophilic groups on a linker reagent include, but are not limited to, hydrazide, oxime, amino, hydrazine, thiosemicarbazone, hydrazine carboxyl, and arylhydrazide. In an embodiment, an antibody (or antigen binding portion thereof or other binding agent (including non-antibody scaffolds)) is modified to introduce electrophilic moieties that are capable of reacting with nucleophilic substituents on a linker. In another embodiment, the sugars of glycosylated antibodies may be oxidized, e.g. with periodate oxidizing reagents, to form aldehyde or ketone groups which may react with the amine group of a linker. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody (or antigen binding portion thereof or other binding agent (including non-antibody scaffolds)) that can react with appropriate groups on the linker (see, e.g., Hermanson, Bioconjugate Techniques). In another embodiment, the EGFR and c-MET bispecific binding agent such as antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a linker.

Exemplary nucleophilic groups on a drug unit, such as a cytotoxic agent, include, but are not limited to: amine, thiol, hydroxyl, hydrazide, oxime, hydrazine, thiosemicarbazone, hydrazine carboxyl, and arylhydrazide groups capable of reacting to form covalent bonds with electrophilic groups on a linker(s) including: (i) active esters such as NHS esters, HOBt esters, haloformates, and acid halides; (ii) alkyl and benzyl halides such as haloacetamides; (iii) aldehydes, ketones, carboxyl, and maleimide groups.

In some embodiments, a drug-linker is attached to an interchain cysteine residue(s) of an antibody (or antigen binding portion thereof or other binding agent (including non-antibody scaffolds)). See, e.g., WO2004/010957 and WO2005/081711. In such embodiments, the linker typically comprises a maleimide group for attachment to the cysteine residues of an interchain disulfide. In some embodiments, a linker or drug-linker is attached to a cysteine residue(s) of an antibody or antigen binding portion thereof as described in U.S. Pat. No. 7,585,491 or 8,080,250. The drug loading of the resulting conjugate typically ranges from 1 to 8 or 1 to 16.

In some embodiments, a linker or drug-linker is attached to a lysine or cysteine residue(s) of an antibody (or antigen binding portion thereof or other binding agent) as described in WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences can be used for site-specific attachment of linkers or drug-linkers to antibodies or antigen binding portions thereof or other binding agents (including non-antibody scaffolds).

In some embodiments, a drug-linker(s) is attached to an engineered cysteine residue at an Fc residue other than an interchain disulfide. In some embodiments, a drug-linker(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, a linker or drug-linker(s) is attached to one or more introduced cysteine residues of an antibody (or antigen binding portion thereof or other binding agent (including non-antibody scaffolds)) as described in WO2006/034488, WO2011/156328 and/or WO2016040856.

In some embodiments, an exemplary substitution for site specific conjugation using bacterial transglutaminase is N297S or N297Q of the Fc region. In some embodiments, a linker or drug-linker(s) is attached to the glycan or modified glycan of an antibody or antigen binding portion or a glycoengineered antibody (or other binding agent (including non-antibody scaffolds)). See, e.g., WO2017/147542, WO2020/123425, WO2020/245229, WO2014/072482; WO2014//065661, WO2015/057066 and WO2016/022027; the disclosure of which are incorporated by reference herein.

In some embodiments, a linker or drug-linker is attached to an antibody, antigen binding portion or other binding agent (including non-antibody scaffolds) via Sortase A linker. A Sortase A linker can be created by a Sortase A enzyme fusing an LPXTG recognition motif (SEQ ID NO: 61) to an N-terminal GGG motif to regenerate a native amide bond.

In some embodiments, a linker or drug-linker is attached to an antibody, antigen binding portion or other binding agent (including non-antibody scaffolds) using SMARTag Technology, in which a bioorthogonal aldehyde handle is introduced through the oxidation of a cysteine residue, embedded in a specific peptide sequence (CxPxR), to an aldehyde-bearing formylglycine (fGly). This enzymatic modification is carried out by the formylglycine-generating enzyme (FGE). See, e.g., Liu et al., Methods Mol. Biol. 2033:131-147 (2019).

In some embodiments, a linker or drug-linker is attached to an antibody, antigen binding portion or other binding agent (including non-antibody scaffolds) using cysteine conjugation with quaternized vinyl- and alkynyl-pyridine reagents. See, e.g., Matos et al., Angew Chem. Int. Ed. Engl. 58:6640-6644 (2019).

In other embodiments, a linker or drug-linker is attached to an antibody, antigen binding portion or other binding agent (including non-antibody scaffolds) using bis-maleimide, C-lock, or K-lock methodologies.

### Pharmaceutical Compositions

Other aspects of the conjugates relate to compositions comprising active ingredients, including any of the conjugates described herein. In some embodiments, the composition is a pharmaceutical composition. As used herein, the term “pharmaceutical composition” refers to an active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use can also be prepared. A preparation can also be emulsified or presented as a liposome composition. A conjugate can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., a conjugate).

The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain the active ingredients (e.g., a conjugate) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising a conjugate can be a lyophilisate.

In some embodiments, a syringe comprising a therapeutically effective amount of a conjugate is provided.

### Treatment Methods

In some embodiments, provided are methods of treating a subject, comprising administering to the subject a conjugate described herein or a pharmaceutical composition described herein. For example, in some embodiments the subject has cancer or an autoimmune disease and the conjugate binds to the target antigen associated with the cancer or autoimmune disease.

In some embodiments, provided are methods of treating cancer comprising administering a conjugate. In some embodiments, the subject is in need of treatment for a cancer and/or a malignancy. In some embodiments, the method is for treating a subject having a cancer or malignancy.

The methods described herein include administering a therapeutically effective amount of a conjugate to a subject having a cancer or malignancy. As used herein, the phrases “therapeutically effective amount”, “effective amount” or “effective dose” refer to an amount of a conjugate that provides a therapeutic benefit in the treatment of, management of or prevention of relapse of a cancer or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell cancer, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic cancer, hepatoma, intra-epithelial neoplasm, kidney or renal cancer (e.g., clear cell cancer), larynx cancer, leukemia, liver cancer, lung cancer (e.g., small-cell lung cancer, non-small cell lung cancer, adenocarcinoma of the lung, and squamous cancer of the lung), lymphoma including Hodgkin's and non-Hodgkin's lymphoma, melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland cancer, sarcoma, skin cancer, squamous cell cancer, testicular cancer, thyroid cancer, uterine or endometrial cancer, uterine serious cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

In some embodiments, the cancer is an EGFR and c-MET cancer. In some embodiments, the EGFR and c-MET cancer is a solid tumor or a hematologic malignancy.

In some embodiments, the EGFR and c-MET cancer is selected from breast cancer, ovarian cancer (OVCA), cervical cancer, pharynx cancer, stomach cancer, myeloma, bladder cancer, uterine cancer, esophageal squamous cell carcinoma (ESCC), colon cancer, hepatocellular cancer, and colorectal cancer.

In some embodiments, the EGFR and c-MET cancer is a hematologic malignancy.

In some embodiments, EGFR and c-MET cancer is a solid tumor. In some embodiments, the EGFR and c-MET cancer is triple-negative breast cancer (TNBC). In some embodiments, the EGFR and c-MET cancer is non-small-cell lung cancer (NSCLC). In some embodiments, the EGFR and c-MET cancer is ovarian teratocarcinoma. In some embodiments, the EGFR and c-MET cancer is pharynx cancer. In some embodiments, the EGFR and c-MET cancer is gastric adenocarcinoma. In some embodiments, the EGFR and c-MET cancer is endometrial adenocarcinoma. In some embodiments, the EGFR and c-MET cancer is bladder transitional cell carcinoma. In some embodiments, the EGFR and c-MET cancer is bladder transitional cell papilloma. In some embodiments, the EGFR and c-MET cancer is ESCC.

It is contemplated that the methods herein reduce tumor size or tumor burden in the subject, and/or reduce metastasis in the subject. In various embodiments, tumor size in the subject is decreased by about 25-50%, about 40-70% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

In some embodiments, a subject can be one who has been previously diagnosed with or identified as suffering from a cancer and in need of treatment, but need not have already undergone treatment for the cancer. In some embodiments, a subject can also be one who has not been previously diagnosed as having a cancer in need of treatment. In some embodiments, a subject can be one who exhibits one or more risk factors for a condition or one or more complications related to a cancer or a subject who does not exhibit risk factors. A “subject in need” of treatment for a cancer particular can be a subject having that condition or diagnosed as having that condition. In other embodiments, a subject “at risk of developing” a condition refers to a subject diagnosed as being at risk for developing the condition or at risk for having the condition again.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in cancer cells in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing a conjugate as described herein to a subject by a method or route which results in binding of the conjugate to cancer cells or malignant cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate can be administered in a unit dose. The term “unit dose” when used in reference to a pharmaceutical composition refers to physically discrete units suitable as unitary dosage for the subject, each unit containing a predetermined quantity of active material (e.g., conjugate), calculated to produce the desired therapeutic effect in association with the required physiologically acceptable diluent, i.e., carrier, or vehicle.

In some embodiments, the conjugates as described herein can be used in a method(s) comprising administering a conjugate to a subject in need thereof, such as a subject having an autoimmune disease.

In some embodiments, provided are methods of treating an autoimmune disease comprising administering a conjugate as described herein. In some embodiments, the subject is in need of treatment for an autoimmune disease. The methods described herein include administering a therapeutically effective amount of a conjugate to a subject having an autoimmune disease. As used herein, the phrase “therapeutically effective amount”, “effective amount” or “effective dose” refers to an amount of a conjugate as described herein that provides a therapeutic benefit in the treatment of, management of or prevention of relapse of an autoimmune disease, e.g., an amount that provides a statistically significant decrease in at least one symptom, sign, or marker of an autoimmune disease. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The term “autoimmune disease” refers to an immunological disorder characterized by inappropriate activation of immune cells (e.g., lymphocytes or dendritic cells), that interferes with the normal functioning of the bodily organs and systems. Examples of autoimmune disease include, but are not limited to, rheumatoid arthritis, psoriatic arthritis, autoimmune demyelinative diseases (e.g., multiple sclerosis, allergic encephalomyelitis), endocrine ophthalmopathy, uveoretinitis, systemic lupus erythematosus, myasthenia gravis, Grave's disease, glomerulonephritis, autoimmune hepatological disorder, inflammatory bowel disease (e.g., Crohn's disease), anaphylaxis, allergic reaction, Sjogren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nodosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Samter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the methods described herein encompass treatment of disorders of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various autoimmune diseases. In addition, the methods described herein can be used to treat domesticated animals and/or pets. A subject can be male or female. In certain embodiments, the subject is a human.

In some embodiments, a subject can be one who has been previously diagnosed with or identified as suffering from an autoimmune disease and in need of treatment, but need not have already undergone treatment for the autoimmune disease. In some embodiments, a subject can also be one who has not been previously diagnosed as having an autoimmune disease in need of treatment. In some embodiments, a subject can be one who exhibits one or more risk factors for a condition or one or more complications related to an autoimmune disease or a subject who does not exhibit risk factors. A “subject in need” of treatment for an autoimmune disease particular can be a subject having that condition or diagnosed as having that condition. In other embodiments, a subject “at risk of developing” a condition refers to a subject diagnosed as being at risk for developing the condition or at risk for having the condition again (e.g., an autoimmune disease).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in autoimmune cells in the subject, alleviation of one or more symptom(s), diminishment of extent of the deficit, stabilized (i.e., not worsening) state of an autoimmune disease, delay or slowing of progression of an autoimmune disease, and an increased lifespan as compared to that expected in the absence of treatment. As used herein, the term “administering,” refers to providing a conjugate as described herein to a subject by a method or route which results in binding of the conjugate to target autoimmune cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of progression of an autoimmune disease or a reduction of symptoms. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate thereof can be administered in a unit dose. The term “unit dose” when used in reference to a pharmaceutical composition refers to physically discrete units suitable as unitary dosage for the subject, each unit containing a predetermined quantity of active material (e.g., a conjugate), calculated to produce the desired therapeutic effect in association with the required physiologically acceptable diluent, i.e., carrier, or vehicle.

In some embodiments, a conjugate, or a pharmaceutical composition of any of these, is administered with an immunosuppressive therapy. In some embodiments, provided is a method of improving treatment outcome in a subject receiving immunosuppressive therapy. The method generally includes administering an effective amount of an immunosuppressive therapy to the subject having an autoimmune disorder; and administering a therapeutically effective amount of a conjugate or a pharmaceutical composition thereof to the subject, wherein the conjugate specifically binds to target autoimmune cells; wherein the treatment outcome of the subject is improved, as compared to administration of the immunotherapy alone. In some embodiments, the conjugate thereof as described herein. In some embodiments, an improved treatment outcome is a decrease in disease progression, an alleviation of one or more symptoms, or the like.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Abbreviations

Boc₂O: di-tert-butyl dicarbonate
Bu₄NBr: Tetrabutylammonium bromide
DCM: dichloromethane
DEA: Diethanolamine
DEAD: diethyl azodicarboxylate
DIPEA: N,N-diisopropylethylamine
DMAP: 4-(Dimethylamino)pyridine
DMF: N,N-dimethylformamide
DMTMM: 4-(4,6-dimethoxy-1,3,5-triazin-2-yl)-4-methyl-morpholinium chloride
DMSO: dimethylsulfoxide
EEDQ: N-Ethoxycarbonyl-2-ethoxy-1,2-dihydroquinoline
ESI: electrospray ionization
HATU: 1-[bis(dimethyamino)methylene]-1H-1,2,3-triazolo[4,5-b]pyridinium 3-oxidhexafluorophosophate)
HOBt: hydroxylbenzotriazole
LCMS: liquid chromatography-mass spectrometry
MeCN: acetonitrile
MeOH: methanol
m-CPBA: meta-chloroperoxybenzoic acid
MTBE: Methyl tert-butyl ether
NMR: nuclear magnetic resonance spectroscopy
Ph₃CCl: Triphenylmethyl chloride
PNPC: bis(4-nitrophenyl) carbonate
PPh₃: triphenylphosphine
TFA: trifluoroacetic acid
THF: tetrahydrofuran
TLC: thin-layer chromatography
TsOH: p-Toluenesulfonic acid

### General Methods

¹H NMR and other NMR spectra were recorded on BrukerAVIII 400 or BrukerAVIII 500. The data were processed with Nuts software or MestReNova software, measuring proton shifts in parts per million (ppm) downfield from an internal standard tetramethyl silane.

HPLC-MS measurement was run on Agilent 1200 HPLC/6100 SQ System using the following conditions:

Method A: Mobile Phase: A: Water (0.01% TFA) B: acetonitrile (0.01% TFA); Gradient Phase: 5% of B increasing to 95% of B in 15 min; Flow Rate: 1.0 mL/min; Column: XBridge C18, 4.6*150 mm, 3.5 μm; Column Temperature: 40° C. Detectors: ADC ELSD, DAD (214 nm and 254 nm), ES-API.

Method B: Mobile Phase: A: Water (0.01% TFA) B: acetonitrile (0.01% TFA); Gradient Phase: 5% of B increasing to 95% of B in 15 min; Flow Rate: 1.0 mL/min; Column: SunFire C18, 4.6*150 mm, 3.5 μm; Column Temperature: 45° C. Detectors: ADC ELSD, DAD (214 nm and 254 nm), ES-API.

Method C: Mobile Phase: A: Water (10 mM NH₄HCO₃) B: acetonitrile; Gradient Phase: 5% to 95% of B in 15 min; Flow Rate: 1.0 mL/min; Column: XBridge C18, 4.6*150 mm, 3.5 μm; Column Temperature: 40° C. Detectors: ADC ELSD, DAD (214 nm and 254 nm), MSD (ES-API).

LCMS measurement was run on Agilent 1200 HPLC/6100 SQ System using the following conditions:

Method A: Mobile Phase: A: Water (0.01% TFA) B: acetonitrile (0.01% TFA); Gradient Phase: 5% of B increasing to 95% of B in 3 min; Flow Rate: 1.8-2.3 mL/min; Column: SunFire C18, 4.6*50 mm, 3.5 μm; Column Temperature: 50° C. Detectors: ADC ELSD, DAD (214 nm and 254 nm), ES-API.

Method B: Mobile Phase: A: Water (10 mM NH₄HCO₃) B: Acetonitrile; Gradient Phase: 5% to 95% of B in 3 min; Flow Rate: 1.8-2.3 mL/min; Column: XBridge C18, 4.6*50 mm, 3.5 μm; Column Temperature: 50° C. Detectors: ADC ELSD, DAD (214 nm and 254 nm), MSD (ES-API).

Preparative high pressure liquid chromatography (Prep-HPLC) was run on Gilson 281 using the following conditions:

Method A: Waters SunFire 10 μm C18 column (100 \acute{Å}, 250×19 mm). Solvent A was water/0.01% trifluoroacetic acid (TFA) and solvent B was acetonitrile. The elution condition was a linear gradient increase of solvent B from 5% to 100% over a time period of 20 min at a flow rate of 30 mL/min.

Method B: Waters SunFire 10 μm C18 column (100 \acute{Å}, 250×19 mm). Solvent A was water/0.05% formic acid (FA) and solvent B was acetonitrile. The elution condition was a linear gradient increase of solvent B from 5% to 100% over a time period of 20 min at a flow rate of 30 mL/min.

Method C: Waters Xbridge 10 μm C18 column (100 \acute{Å}, 250×19 mm). Solvent A was water/10 mM ammonium bicarbonate (NH₄HCO₃) and solvent B was acetonitrile. The elution condition was a linear gradient increase of solvent B from 5% to 100% over a time period of 20 minutes at a flow rate of 30 mL/min.

Flash chromatography was performed on instrument of Biotage, with Agela Flash Column silica-CS; Reverse phase flash chromatography was performed on instrument of Biotage, with Boston ODS or Agela C18.

### Example 1: Preparation of Bsab 67-LD038 (5)

The aqueous of 10 mM TCEP HCl (Tris(2-carboxyethyl) phosphine HCl) was added to 1 mL of antibody (25 mg/mL) in 20 mM Histidine buffer (pH=5.5), at the molar ratio of TCEP to mAb is 2.5. Reducing reaction was conducted for 3 h at 24° C. LD038 (salt of TFA) was dissolved in water at a concentration of 20 mg/mL and added to reduced mAb at a molar ratio of 4.8 (LD038/mAb). The coupling reaction was stirred for 1.5 h at 24° C. The excess LD038 and its impurities were removed by ultrafiltration with 20 mM Histidine buffer (pH=5.5). The ADC was stored in 20 mM histidine buffer containing 6% sucrose and 0.02% (w/V) Tween 20 by ultrafiltration/diafiltration (UFDF). The purity of ADC as determined by SEC-HPLC was 99.0% and DAR value as determined by LC-MS was 5.1.

### Example 2: Preparation of AZD9592

The aqueous of 10 mM TCEP HCl (Tris(2-carboxyethyl) phosphine HCl) was added to 5.5 mL of antibody (7.65 mg/mL) in 20 mM Histidine buffer (pH=6.0), at the molar ratio of TCEP to mAb is 8.0. Reducing reaction was conducted for 2 h at 24° C. The excess TCEP and its byproduct was removed by ultrafiltration with 20 mM Histidine buffer (pH=5.5). SG3932 was dissolved in water at a concentration of 10 mM and added to reduced mAb at a molar ratio of 6 (SG3932/mAb). The coupling reaction was stirred for 1 h at 24° C. The excess SG3932 and its impurities were removed by ultrafiltration with 20 mM Histidine buffer (pH=5.5). The ADC was stored in 20 mM histidine buffer containing 6% sucrose and 0.02% (w/V) Tween 20 by UFDF. The purity of ADC as determined by SEC-HPLC was 98.22% and DAR value as determined by LC-MS was 5.9.

### Example 3: Preparation of b12-LD038 (5)

The aqueous of 10 mM TCEP HCl (Tris(2-carboxyethyl) phosphine HCl) was added to 3 mL of antibody (10 mg/mL) in 20 mM Histidine buffer (pH=5.5), at the molar ratio of TCEP to mAb is 2.7. Reducing reaction was conducted for 3 h at 24° C. LD038 (salt of TFA) was dissolved in water at a concentration of 20 mg/mL and added to reduced mAb at a molar ratio of 5 (LD038/mAb). The coupling reaction was stirred for 1.5 h at 24° C. The excess LD038 and its impurities were removed by ultrafiltration with 20 mM Histidine buffer (pH=5.5). The ADC was stored in 20 mM histidine buffer containing 6% sucrose and 0.02% (w/V) Tween 20 by UFDF. The purity of ADC as determined by SEC-HPLC was 98.0% and DAR value as determined by LC-MS was 5.0.

### Example 4: Preparation of b12-LD038 (8)

The aqueous of 10 mM TCEP HCl (Tris(2-carboxyethyl) phosphine HCl) was added to 3 mL of antibody (10 mg/mL) in 20 mM Histidine buffer (pH=5.5), at the molar ratio of TCEP to mAb is 12. Reducing reaction was conducted for 3 h at 24° C. The excess TCEP and its byproduct was removed by ultrafiltration with pH=20 mM Histidine buffer. LD038 (salt of TFA) was dissolved in water at a concentration of 20 mg/mL and added to reduced mAb at a molar ratio of 9 (LD038/mAb). The coupling reaction was stirred for 1.5 h at 24° C. The excess LD038 and its impurities were removed by ultrafiltration with 20 mM Histidine buffer (pH=5.5). The ADC was stored in 20 mM histidine buffer containing 6% sucrose and 0.02% (w/N) Tween 20 by UFDF. The purity of ADC as determined by SEC-HPLC was 98.0% and DAR value as determined by LC-MS was 7.9.

### Example 5: Preparation of Bsab 67-LD038 (8)

The aqueous of 10 mM TCEP HCl (Tris(2-carboxyethyl) phosphine HCl) was added to 1 mL of antibody (25 mg/mL) in 20 mM Histidine buffer (pH=5.5), at the molar ratio of TCEP to mAb is 12. Reducing reaction was conducted for 3 h at 24° C. The excess TCEP and its byproduct was removed by ultrafiltration with pH=20 mM Histidine buffer. LD038 (salt of TFA) was dissolved in water at a concentration of 20 mg/mL and added to reduced mAb at a molar ratio of 9 (LD038/mAb). The coupling reaction was stirred for 1.5 h at 24° C. The excess LD038 and its impurities were removed by ultrafiltration with 20 mM Histidine buffer (pH=5.5). The ADC was stored in 20 mM histidine buffer containing 6% sucrose and 0.02% (w/N) Tween 20 by UFDF. The purity of ADC as determined by SEC-HPLC was 99.0% and DAR value as determined by LC-MS was 8.1.

### Example 6: Preparation of Bsab 67-LD343 (8)

The 2 mL of antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb is 8.0. Reducing reaction was conducted for 2 h at 25° C. The excess TCEP and its byproduct was removed by ultrafiltration with pH=6.9 50 mM sodium phosphate buffer. LD343 (salt of TFA) was dissolved in water at a concentration of 20 mg/mL and added to reduced mAb at a molar ratio of 8.5 (LD343/mAb). The coupling reaction was stirred for 2 h at 25° C. The excess LD343 and its impurities were removed by ultrafiltration with 50 mM sodium phosphate buffer. The ADC was stored in 20 mM histidine buffer containing 6% sucrose and 0.02% (w/V) Tween 20 by UFDF. The purity of ADC as determined by SEC-HPLC was 98.0% and DAR value as determined by LC-MS was 7.9.

### Example 7: Preparation of b12-LD343 (8)

The 2 mL of antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb is 8.0. Reducing reaction was conducted for 2 h at 25° C. The excess TCEP and its byproduct was removed by ultrafiltration with pH=6.9 50 mM sodium phosphate buffer. LD343 (salt of TFA) was dissolved in water at a concentration of 20 mg/mL and added to reduced mAb at a molar ratio of 8.5 (LD343/mAb). The coupling reaction was stirred for 2 h at 25° C. The excess LD343 and its impurities were removed by ultrafiltration with 50 mM sodium phosphate buffer. The ADC was stored in 20 mM histidine buffer containing 6% sucrose and 0.02% (w/V) Tween 20 by UFDF. The purity of ADC as determined by SEC-HPLC was 98.0% and DAR value as determined by LC-MS was 7.5.

### Example 8: Binding Activity of Parent mAbs

EGFR and cMET co-expressed tumor cell lines including A431 (ATCC® CRL-1555), A549 (ATCC® CCL-185, Provided by Procell), MDA-MB-468 (ATCC® HTB-132, Provided by Procell), MKN-45 (CL-0292, Provided by Procell), NCI-H441 (ATCC® HTB-174, Provided by COBIOER), NCI-H1975 (ATCC® CRL-5908, Provided by Procell) were selected for evaluating antibody binding activity by flow cytometry.

A431 was cultured with DMEM medium (Gibco, Cat #C11995500BT) containing 10% FBS. MDA-MB-468, MKN-45, NCI-H441 and NCI-H1975 were cultured with RPMI 1640 medium (Gibco, Cat #11875093) containing 10% FBS. A549 was cultured with F12K medium (Gibco, Cat #21127030) containing 10% FBS. Each antibody was incubated with different cell lines (1×10⁵ cells/well) for 30 min in 0.2 mL FACS buffer (1×PBS with 0.1% BSA) at 4° C. After wash, cells were incubated with 100 μL PE-conjugated anti human Fc (Abcam, Ab98596, 1:500 dilution) for 30 min at 4° C. After wash with PBS, cells were resuspended in FACS buffer and analyzed by flow cytometer (Beckman, CytoFLEX).

Sequences of the variable regions and CDRs of parent cMET mAbs, parent EGFR mAbs, and bispecific positive controls are shown in Tables 1-3.

TABLE 1
Sequences of parent cMET mAbs

| | | | SEQ ID |
|---|---|---|---|
| Antibody | Description | Amino Acid Sequence | NO: |
| Onartuzumab | VH | EVQLVESGGGLVQPGGSLRLSCAASGYTFTSY | 1 |
| | | WLHWVRQAPGKGLEWVGMIDPSNSDTRFNPN | |
| | | FKDRFTISADTSKNTAYLQMNSLRAEDTAVYYCA | |
| | | TYRSYVTPLDYWGQGTLVTVSS | |
| | VL | DIQMTQSPSSLSASVGDRVTITCKSSQSLLYTSS | 2 |
| | | QKNYLAWYQQKPGKAPKLLIYWASTRESGVPS | |
| | | RFSGSGSGTDFTLTISSLQPEDFATYYCQQYYAY | |
| | | PWTFGQGTKVEIK | |
| | HCDR1 | GYTFTSYW | 3 |
| | HCDR2 | IDPSNSDT | 4 |
| | HCDR3 | ATYRSYVTPLDY | 5 |
| | LCDR1 | QSLLYTSSQKNY | 6 |
| | LCDR2 | WAS | |
| | LCDR3 | QQYYAYPWT | 7 |
| Telisotuzumab | VH | QVQLVQSGAEVKKPGASVKVSCKASGYIFTAYT | 8 |
| | | MHWVRQAPGQGLEWMGWIKPNNGLANYAQKF | |
| | | QGRVTMTRDTSISTAYMELSRLRSDDTAVYYCA | |
| | | RSEITTEFDYWGQGTLVTVSS | |
| | VL | DIVMTQSPDSLAVSLGERATINCKSSESVDSYAN | 9 |
| | | SFLHWYQQKPGQPPKLLIYRASTRESGVPDRF | |
| | | SGSGSGTDFTLTISSLQAEDVAVYYCQQSKEDP | |
| | | LTFGGGTKVEIK | |
| | HCDR1 | GYIFTAYT | 10 |
| | HCDR2 | IKPNNGLA | 11 |
| | HCDR3 | ARSEITTEFDY | 12 |
| | LCDR1 | ESVDSYANSF | 13 |
| | LCDR2 | RAS | |
| | LCDR3 | QQSKEDPLT | 14 |
| Emibetuzumab | VH | QVQLVQSGAEVKKPGASVKVSCKASGYTFTDY | 15 |
| | | YMHWVRQAPGQGLEWMGRVNPNRRGTTYNQ | |
| | | KFEGRVTMTTDTSTSTAYMELRSLRSDDTAVYY | |
| | | CARANWLDYWGQGTTVTVSS | |
| | VL | DIQMTQSPSSLSASVGDRVTITCSVSSSVSSIYL | 16 |
| | | HWYQQKPGKAPKLLIYSTSNLASGVPSRFSGS | |
| | | GSGTDFTLTISSLQPEDFATYYCQVYSGYPLTFG | |
| | | GGTKVEIK | |
| | HCDR1 | GYTFTDYY | 17 |
| | HCDR2 | VNPNRRGT | 18 |
| | HCDR3 | ARANWLDY | 19 |
| | LCDR1 | SSVSSIY | 20 |
| | LCDR2 | STS | |
| | LCDR3 | QVYSGYPLT | 21 |
| Nova-5091 | VH | QVQLVQSGAEVKKPGASVKVSCKASGYTFTGY | 22 |
| | | YMNWVRQAPGQGLEWMGIINPWTGNTNYAQK | |
| | | FQGRVTMTRDTSISTAYMELSSLRSEDTAVYYC | |
| | | ARDPGFFYYTPSDLWGQGTLVTVSS | |
| | VL | DIELTQPPSVSVAPGQTARISCSGDSIGNKYVHW | 23 |
| | | YQQKPGQAPVLVIYADSDRPSGIPERFSGSNSG | |
| | | NTATLTISGTQAEDEADYYCQSYAHYHDIWVFG | |
| | | GGTKLTVLG | |
| | HCDR1 | GYTFTGYY | 24 |
| | HCDR2 | INPWTGNT | 25 |
| | HCDR3 | ARDPGFFYYTPSDL | 26 |
| | LCDR1 | SIGNKY | 27 |
| | LCDR2 | ADS | |
| | LCDR3 | QSYAHYHDIWV | 28 |
| Nova-5097 | VH | QVQLVQSGAEVKKPGSSVKVSCKASGGTFSSY | 29 |
| | | AISWVRQAPGQGLEWMGGIDPFGTANYAQKFQ | |
| | | GRVTITADESTSTAYMELSSLRSEDTAVYYCARV | |
| | | YQDVWGQGTLVTVSS | |
| | VL | DIVMTQSPDSLAVSLGERATINCRSSQSILYGINN | 30 |
| | | NFLGWYQQKPGQPPKLLIYWASTRESGVPDRF | |
| | | SGSGSGTDFTLTISSLQAEDVAVYYCQQYAFGW | |
| | | TFGQGTKVEIK | |
| | HCDR1 | GGTFSSYA | 31 |
| | HCDR2 | IDPFGTA | 32 |
| | HCDR3 | ARVYQDV | 33 |
| | LCDR1 | QSILYGINNNF | 34 |
| | LCDR2 | WAS | |
| | LCDR3 | QQYAFGWT | 35 |
| Nova-5098 | VH | QVQLVQSGAEVKKPGSSVKVSCKASGGTFSSY | 36 |
| | | AISWVRQAPGQGLEWMGGIDPFGTANYAQKFQ | |
| | | GRVTITADESTSTAYMELSSLRSEDTAVYYCARV | |
| | | YQDVWGQGTLVTVSS | |
| | VL | DIVMTQSPDSLAVSLGERATINCRSSQSILYGINN | 37 |
| | | NFLGWYQQKPGQPPKLLIYWASTRESGVPDRF | |
| | | SGSGSGTDFTLTISSLQAEDVAVYYCLQYSDEP | |
| | | WTFGQGTKVEIK | |
| | HCDR1 | GGTFSSYA | 38 |
| | HCDR2 | IDPFGTA | 39 |
| | HCDR3 | ARVYQDV | 40 |
| | LCDR1 | QSILYGINNNF | 41 |
| | LCDR2 | WAS | |
| | LCDR3 | LQYSDEPWT | 42 |
| Nova-5185 | VH | QVQLVQSGAEVKKPGSSVKVSCKASGGTFSSY | 43 |
| | | AISWVRQAPGQGLEWMGGIDPIMGTEYAQKFQ | |
| | | GRVTITADESTSTAYMELSSLRSEDTAVYYCARV | |
| | | YQDVWGQGTLVTVSS | |
| | VL | DIVMTQSPDSLAVSLGERATINCRSSQSILYGINN | 44 |
| | | NFLGWYQQKPGQPPKLLIYWASTRESGVPDRF | |
| | | SGSGSGTDFTLTISSLQAEDVAVYYCQQYAYEP | |
| | | NTFGQGTKVEIK | |
| | HCDR1 | GGTFSSYA | 45 |
| | HCDR2 | IDPIMGT | 46 |
| | HCDR3 | ARVYQDV | 47 |
| | LCDR1 | QSILYGINNNF | 48 |
| | LCDR2 | WAS | |
| | LCDR3 | QQYAYEPNT | 49 |
| ABF-46 | VH | EVKLVESGGGLVQPGGSLRLSCATSGFTFTDYY | 50 |
| | | MSWVRQPPGKALEWLGFIRNKANGYTTEYSAS | |
| | | VKGRFTISRDNSQSILYLQMDTLRAEDSATYYCA | |
| | | RDNWFAYWGQGTLVTVSA | |
| | VL | DILMTQSPSSLTVSAGEKVTMSCKSSQSLLASG | 51 |
| | | NQNNYLAWHQQKPGRSPKMLIIWASTRVSGVP | |
| | | DRFIGSGSGTDFTLTINSVQAEDLAVYYCQQSY | |
| | | SAPLTFGAGTKLELK | |
| | HCDR1 | GFTFTDYY | 52 |
| | HCDR2 | IRNKANGYTT | 53 |
| | HCDR3 | ARDNWFAY | 54 |
| | LCDR1 | QSLLASGNQNNY | 55 |
| | LCDR2 | WAS | |
| | LCDR3 | QQSYSAPLT | 56 |
| Amivantamab- | VH | QVQLVQSGAEVKKPGASVKVSCETSGYTFTSY | 57 |
| cMET arm | | GISWVRQAPGHGLEWMGWISAYNGYTNYAQKL | |
| | | QGRVTMTTDTSTSTAYMELRSLRSDDTAVYYCA | |
| | | RDLRGTNYFDYWGQGTLVTVSS | |
| | VL | DIQMTQSPSSVSASVGDRVTITCRASQGISNWL | 58 |
| | | AWFQHKPGKAPKLLIYAASSLLSGVPSRFSGSG | |
| | | SGTDFTLTISSLQPEDFATYYCQQANSFPITFGQ | |
| | | GTRLEIK | |
| | HCDR1 | GYTFTSYG | 59 |
| | HCDR2 | ISAYNGYT | 60 |
| | HCDR3 | ARDLRGTNYFDY | 61 |
| | LCDR1 | QGISNW | 62 |
| | LCDR2 | AAS | |
| | LCDR3 | QQANSFPIT | 63 |

TABLE 2
Sequences of parent EGFR mAbs

| | | | SEQ ID |
|---|---|---|---|
| Antibody | Description | Amino Acid Sequence | NO: |
| Cetuximab | VH | QVQLKQSGPGLVQPSQSLSITCTVSGFSLTNYG | 64 |
| | | VHWVRQSPGKGLEWLGVIWSGGNTDYNTPFT | |
| | | SRLSINKDNSKSQVFFKMNSLQSNDTAIYYCAR | |
| | | ALTYYDYEFAYWGQGTLVTVSA | |
| | VL | DILLTQSPVILSVSPGERVSFSCRASQSIGTNIH | 65 |
| | | WYQQRTNGSPRLLIKYASESISGIPSRFSGSGS | |
| | | GTDFTLSINSVESEDIADYYCQQNNNWPTTFGA | |
| | | GTKLELK | |
| | HCDR1 | GFSLTNYG | 66 |
| | HCDR2 | IWSGGNT | 67 |
| | HCDR3 | ARALTYYDYEFAY | 68 |
| | LCDR1 | QSIGTN | 69 |
| | LCDR2 | YAS | |
| | LCDR3 | QQNNNWPTT | 70 |
| Panitumumab | VH | QVQLQESGPGLVKPSETLSLTCTVSGGSVSSG | 71 |
| | | DYYWTWIRQSPGKGLEWIGHIYYSGNTNYNPSL | |
| | | KSRLTISIDTSKTQFSLKLSSVTAADTAIYYCVRD | |
| | | RVTGAFDIWGQGTMVTVSS | |
| | VL | DIQMTQSPSSLSASVGDRVTITCQASQDISNYLN | 72 |
| | | WYQQKPGKAPKLLIYDASNLETGVPSRFSGSG | |
| | | SGTDFTFTISSLQPEDIATYFCQHFDHLPLAFGG | |
| | | GTKVEIK | |
| | HCDR1 | GGSVSSGDYY | 73 |
| | HCDR2 | IYYSGNT | 74 |
| | HCDR3 | VRDRVTGAFDI | 75 |
| | LCDR1 | QDISNY | 76 |
| | LCDR2 | DAS | |
| | LCDR3 | QHFDHLPLA | 77 |
| Nimotuzumab | VH | QVQLQQSGAEVKKPGSSVKVSCKASGYTFTNY | 78 |
| | | YIYWVRQAPGQGLEWIGGINPTSGGSNFNEKFK | |
| | | TRVTITADESSTTAYMELSSLRSEDTAFYFCTRQ | |
| | | GLWFDSDGRGFDFWGQGTTVTVSS | |
| | VL | DIQMTQSPSSLSASVGDRVTITCRSSQNIVHSN | 79 |
| | | GNTYLDWYQQTPGKAPKLLIYKVSNRFSGVPS | |
| | | RFSGSGSGTDFTFTISSLQPEDIATYYCFQYSHV | |
| | | PWTFGQGTKLQIT | |
| | HCDR1 | GYTFTNYY | 80 |
| | HCDR2 | INPTSGGS | 81 |
| | HCDR3 | TRQGLWFDSDGRGFDF | 82 |
| | LCDR1 | QNIVHSNGNTY | 83 |
| | LCDR2 | KVS | |
| | LCDR3 | FQYSHVPWT | 84 |
| Zalutumumab | VH | QVQLVESGGGVVQPGRSLRLSCAASGFTFSTY | 85 |
| | | GMHWVRQAPGKGLEWVAVIWDDGSYKYYGDS | |
| | | VKGRFTISRDNSKNTLYLQMNSLRAEDTAVYYC | |
| | | ARDGITMVRGVMKDYFDYWGQGTLVTVSS | |
| | VL | AIQLTQSPSSLSASVGDRVTITCRASQDISSALV | 86 |
| | | WYQQKPGKAPKLLIYDASSLESGVPSRFSGSES | |
| | | GTDFTLTISSLQPEDFATYYCQQFNSYPLTFGG | |
| | | GTKVEIK | |
| | HCDR1 | GFTFSTYG | 87 |
| | HCDR2 | IWDDGSYK | 88 |
| | HCDR3 | ARDGITMVRGVMKDYFDY | 89 |
| | LCDR1 | QDISSA | 90 |
| | LCDR2 | DAS | |
| | LCDR3 | QQFNSYPLT | 91 |
| Necitumumab | VH | QVQLQESGPGLVKPSQTLSLTCTVSGGSISSGD | 92 |
| | | YYWSWIRQPPGKGLEWIGYIYYSGSTDYNPSLK | |
| | | SRVTMSVDTSKNQFSLKVNSVTAADTAVYYCA | |
| | | RVSIFGVGTFDYWGQGTLVTVSS | |
| | VL | EIVMTQSPATLSLSPGERATLSCRASQSVSSYL | 93 |
| | | AWYQQKPGQAPRLLIYDASNRATGIPARFSGSG | |
| | | SGTDFTLTISSLEPEDFAVYYCHQYGSTPLTFG | |
| | | GGTKAEIK | |
| | HCDR1 | GGSISSGDYY | 94 |
| | HCDR2 | IYYSGST | 95 |
| | HCDR3 | ARVSIFGVGTFDY | 96 |
| | LCDR1 | QSVSSY | 97 |
| | LCDR2 | DAS | |
| | LCDR3 | HQYGSTPLT | 98 |
| MRG003 | VH | QVQLQESGPGLVKPSETLSLTCTVSGFSLSNYD | 99 |
| | | VHWVRQAPGKGLEWLGVIWSGGNTDYNTPFT | |
| | | SRLTISVDTSKNQFSLKLSSVTAADTAVYYCARA | |
| | | LDYYDYEFAYWGQGTLVTVSS | |
| | VL | EIVLTQSPDFQSVTPKEKVTITCRASQSIGTNIH | 100 |
| | | WYQQKPDQSPKLLIKYASESISGIPSRFSGSGS | |
| | | GTDFTLTINSLEAEDAATYYCQQNNEWPTSFGQ | |
| | | GTKLEIK | |
| | HCDR1 | GFSLSNYD | 101 |
| | HCDR2 | IWSGGNT | 102 |
| | HCDR3 | ARALDYYDYEFAY | 103 |
| | LCDR1 | QSIGTN | 104 |
| | LCDR2 | YAS | |
| | LCDR3 | QQNNEWPTS | 105 |

TABLE 3
Sequences of bispecific positive control, Amivantamab

| | | | SEQ ID |
|---|---|---|---|
| Antibody | Description | Amino Acid Sequence | NO: |
| Amivantamab | Anti EGFR | QVQLVESGGGVVQPGRSLRLSCAASGFTFSTY | 106 |
| | heavy chain | GMHWVRQAPGKGLEWVAVIWDDGSYKYYGDS | |
| | | VKGRFTISRDNSKNTLYLQMNSLRAEDTAVYYC | |
| | | ARDGITMVRGVMKDYFDYWGQGTLVTVSSAST | |
| | | KGPSVFPLAPSSKSTSGGTAALGCLVKDYFPEP | |
| | | VTVSWNSGALTSGVHTFPAVLQSSGLYSLSSVV | |
| | | TVPSSSLGTQTYICNVNHKPSNTKVDKRVEPKS | |
| | | CDKTHTCPPCPAPELLGGPSVFLFPPKPKDTLM | |
| | | ISRTPEVTCVVVDVSHEDPEVKFNWYVDGVEV | |
| | | HNAKTKPREEQYNSTYRVVSVLTVLHQDWLNG | |
| | | KEYKCKVSNKALPAPIEKTISKAKGQPREPQVYT | |
| | | LPPSREEMTKNQVSLTCLVKGFYPSDIAVEWES | |
| | | NGQPENNYKTTPPVLDSDGSFLLYSKLTVDKSR | |
| | | WQQGNVFSCSVMHEALHNHYTQKSLSLSPGK | |
| | Anti EGFR | AIQLTQSPSSLSASVGDRVTITCRASQDISSALV | 107 |
| | light chain | WYQQKPGKAPKLLIYDASSLESGVPSRFSGSES | |
| | | GTDFTLTISSLQPEDFATYYCQQFNSYPLTFGG | |
| | | GTKVEIKRTVAAPSVFIFPPSDEQLKSGTASWVC | |
| | | LLNNFYPREAKVQWKVDNALQSGNSQESVTEQ | |
| | | DSKDSTYSLSSTLTLSKADYEKHKVYACEVTHQ | |
| | | GLSSPVTKSFNRGEC | |
| | Anti EGFR | GFTFSTYG | 108 |
| | HCDR1 | | |
| | Anti EGFR | IWDDGSYK | 109 |
| | HCDR2 | | |
| | Anti EGFR | ARDGITMVRGVMKDYFDY | 110 |
| | HCDR3 | | |
| | Anti EGFR | QDISSA | 111 |
| | LCDR1 | | |
| | Anti EGFR | DAS | |
| | LCDR2 | | |
| | Anti EGFR | QQFNSYPLT | 112 |
| | LCDR3 | | |
| | Anti cMET | QVQLVQSGAEVKKPGASVKVSCETSGYTFTSY | 113 |
| | heavy chain | GISWVRQAPGHGLEWMGWISAYNGYTNYAQK | |
| | | LQGRVTMTTDTSTSTAYMELRSLRSDDTAVYYC | |
| | | ARDLRGTNYFDYWGQGTLVTVSSASTKGPSVF | |
| | | PLAPSSKSTSGGTAALGCLVKDYFPEPVTVSW | |
| | | NSGALTSGVHTFPAVLQSSGLYSLSSVVTVPSS | |
| | | SLGTQTYICNVNHKPSNTKVDKRVEPKSCDKTH | |
| | | TCPPCPAPELLGGPSVFLFPPKPKDTLMISRTPE | |
| | | VTCVVVDVSHEDPEVKFNWYVDGVEVHNAKTK | |
| | | PREEQYNSTYRVVSVLTVLHQDWLNGKEYKCK | |
| | | VSNKALPAPIEKTISKAKGQPREPQVYTLPPSRE | |
| | | EMTKNQVSLTCLVKGFYPSDIAVEWESNGQPE | |
| | | NNYKTTPPVLDSDGSFFLYSRLTVDKSRWQQG | |
| | | NVFSCSVMHEALHNHYTQKSLSLSPGK | |
| | Anti cMET | DIQMTQSPSSVSASVGDRVTITCRASQGISNWL | 114 |
| | light chain | AWFQHKPGKAPKLLIYAASSLLSGVPSRFSGSG | |
| | | SGTDFTLTISSLQPEDFATYYCQQANSFPITFGQ | |
| | | GTRLEIKRTVAAPSVFIFPPSDEQLKSGTASVVC | |
| | | LLNNFYPREAKVQWKVDNALQSGNSQESVTEQ | |
| | | DSKDSTYSLSSTLTLSKADYEKHKVYACEVTHQ | |
| | | GLSSPVTKSFNRGEC | |
| | Anti cMET | GYTFTSYG | 115 |
| | HCDR1 | | |
| | Anti cMET | ISAYNGYT | 116 |
| | HCDR2 | | |
| | Anti cMET | ARDLRGTNYFDY | 117 |
| | HCDR3 | | |
| | Anti cMET | QGISNW | 118 |
| | LCDR1 | | |
| | Anti cMET | AAS | |
| | LCDR2 | | |
| | Anti cMET | QQANSFPIT | 119 |
| | LCDR3 | | |
| AZD9592-Ab | Anti EGFR | QVQLVQSGAEVKKPGSSVKVSCKASGGTFSDN | 120 |
| (RAA22/B09- | heavy chain | DFSWVRQAPGQGLEWMGAIVAVFRTETYAQKF | |
| TM) | | QDRVKITADISTRTTYMELSSLRSEDTAVYYCAR | |
| | | RLMSAISGPGAPLLMWGQGTLVTVSSASTKGP | |
| | | SVCPLAPSSKSTSGGTAALGCLVKDYFPEPVTV | |
| | | SWNSGALTSGVHTFPAVLQSSGLYSLSSVVTVP | |
| | | SSSLGTQTYICNVNHKPSNTKVDKRVEPKSVDK | |
| | | THTCPPCPAPEFEGGPSVFLFPPKPKDTLMISR | |
| | | TPEVTCVVVDVSHEDPEVKFNWYVDGVEVHNA | |
| | | KTKPREEQYNSTYRVVSVLTVLHQDWLNGKEY | |
| | | KCKVSNKALPASIEKTISKAKGQPREPQVYTLPP | |
| | | CREEMTKNQVSLWCLVKGFYPSDIAVEWESNG | |
| | | QPENNYKTTPPVLDSDGSFFLYSKLTVDKSRW | |
| | | QQGNVFSCSVMHEALHNHYTQKSLSLSPGK | |
| | Anti EGFR | QSALTQPRSVSGSPGQSVTISCTGTSSDVGGY | 121 |
| | light chain | NYVSWYQQHPGKAPKLMIYDVSKRPSGVPDRF | |
| | | SGSKSGNTASLTISGLQAEDEADYYCSSYTSSD | |
| | | TLEIFGGGTKLTVLGQPKAAPSVTLFPPCSEELQ | |
| | | ANKATLVCLISDFYPGAVTVAWKADSSPVKAGV | |
| | | ETTTPSKQSNNKYAASSYLSLTPEQWKSHRSY | |
| | | SCQVTHEGSTVEKTVAPTEVS | |
| | Anti EGFR | GGTFSDND | 122 |
| | HCDR1 | | |
| | Anti EGFR | IVAVFRTE | 123 |
| | HCDR2 | | |
| | Anti EGFR | ARRLMSAISGPGAPLLM | 124 |
| | HCDR3 | | |
| | Anti EGFR | SSDVGGYNY | 125 |
| | LCDR1 | | |
| | Anti EGFR | DVS | |
| | LCDR2 | | |
| | Anti EGFR | SSYTSSDTLEI | 126 |
| | LCDR3 | | |
| | Anti cMET | QVQLVQSGAEVKKPGASVKVSCKASGYTFTDY | 127 |
| | heavy chain | YIHWVRQATGQGLEWMGWMNPNSGNTGYAQ | |
| | | KFQGRVTMTRDTSISTAYMELSSLRSEDTAVYY | |
| | | CARGQGYTHSWGQGTMVTVSSASTKGPSVFP | |
| | | LAPSSKSTSGGTAALGCLVKDYFPEPVTVSWN | |
| | | SGALTSGVHTFPAVLQSSGLYSLSSVVTVPSSS | |
| | | LGTQTYICNVNHKPSNTKVDKRVEPKSCDKTHT | |
| | | CPPCPAPEFEGGPSVFLFPPKPKDTLMISRTPE | |
| | | VTCVVVDVSHEDPEVKFNWYVDGVEVHNAKTK | |
| | | PREEQYNSTYRVVSVLTVLHQDWLNGKEYKCK | |
| | | VSNKALPASIEKTISKAKGQPREPQVCTLPPSRE | |
| | | EMTKNQVSLSCAVKGFYPSDIAVEWESNGQPE | |
| | | NNYKTTPPVLDSDGSFFLVSKLTVDKSRWQQG | |
| | | NVFSCSVMHEALHNHYTQKSLSLSPGK | |
| | Anti cMET | DIQMTQSPSTLSASVGDRVTITCRASEGIYHWL | 128 |
| | light chain | AWYQQKPGKAPKLLIYKASSLASGVPSRFSGS | |
| | | GSGTEFTLTISSLQPDDFATYYCQQYSNYPPTF | |
| | | GGGTKLEIKRTVAAPSVFIFPPSDEQLKSGTASV | |
| | | VCLLNNFYPREAKVQWKVDNALQSGNSQESVT | |
| | | EQDSKDSTYSLSSTLTLSKADYEKHKVYACEVT | |
| | | HQGLSSPVTKSFNRGEC | |
| | Anti cMET | GYTFTDYY | 129 |
| | HCDR1 | | |
| | Anti cMET | MNPNSGNT | 130 |
| | HCDR2 | | |
| | Anti cMET | ARGQGYTHS | 131 |
| | HCDR3 | | |
| | Anti cMET | EGIYHW | 132 |
| | LCDR1 | | |
| | Anti cMET | KAS | |
| | LCDR2 | | |
| | Anti cMET | QQYSNYPPT | 133 |
| | LCDR3 | | |

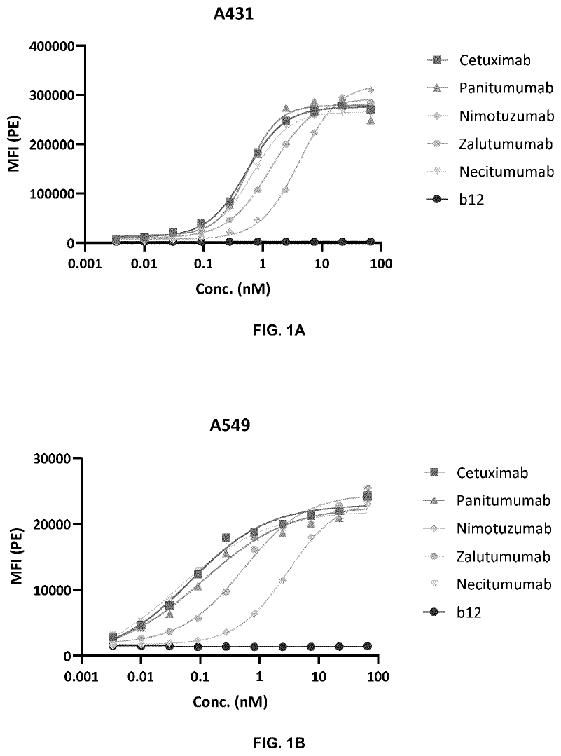

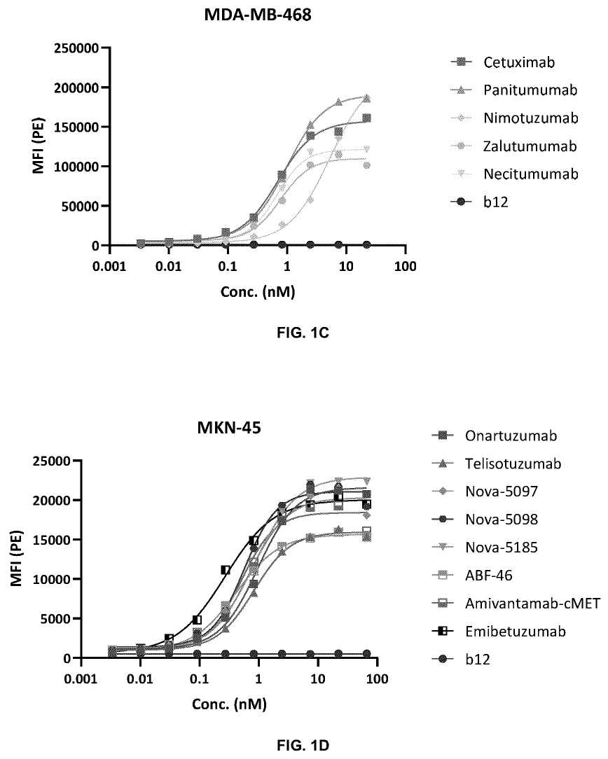

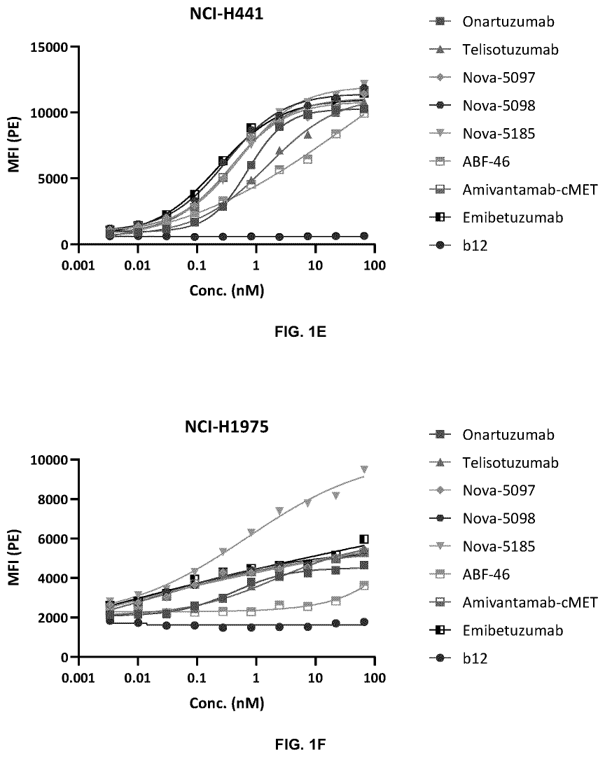

The results of binding activity of parent mAbs in various tumor cell lines were as shown in FIGS. 1A-1F and were summarized in the Tables 4-5. All EGFR parent mAbs and onartuzumab, telisotuzumab and emibetuzumab have been used for bispecific construction.

TABLE 4
cMET sequence

| | |
|---|---|
| Telisotuzumab | Final choice |
| Onartuzumab | Stronger binding than teliso, tumor promoting activity due to bivalent binding |
| | (Roche advanced a monovalent version into clinic), inferior choice |
| ABF-46 | Lower activity than teliso, mouse antibody, filed in 2010; inferior choice |
| Emibetuzumab | Strongest cell binding among all, even stronger than ami's cMET, not fit for |
| | bispecific MOA |
| Nova-5091 | No binding activity |
| Nova-5097 | Stronger binding than teliso, no clinical validation |
| Nova-5098 | Stronger binding than teliso, no clinical validation |
| Nova-5185 | Stronger binding than teliso, seems to have off-target binding |

TABLE 5
EGFR sequence

| | |
|---|---|
| Zalutumumab | Final choice |
| Cetuximab | Strong affinity, chimeric, binding remains |
| | strong as Bsab, not fit for bispecific MOA |
| Panitumumab | Stronger affinity than cetuximab, binding remains |
| | strong as Bsab, not fit for bispecific MOA |
| Nectitumumab | Stronger affinity than zalu, |
| Nimotuzumab | Lower affinity than zalutumumab, no significant |
| | binding differentiation than zalu as Bsab, worse |
| | one step purity, choose zalu due to better |
| | clinical validation |

### Example 9: In Vitro Cytotoxicity of Parent mAbs

Cells were harvested and seeded into 96-well plates (Coring, 3917) for overnight culture. In the next day, serial diluted testing articles (Cetuximab-LD038 (8), Panitumumab-LD038 (8), Nimotuzumab-LD038 (8), Zalutumumab-LD038 (8), Necitumumab-LD038 (8), MRG003-LD038 (8), Onartuzumab-LD038 (8), Telisotuzumab-LD038 (8), Amivantamab-cMET-LD038 (8), Emibetuzumab-LD038 (8) and b12-LD038 (8)) were added to cells and the incubation was lasting for 144 h at 37° C. After that, 40 μL CellTiter-Glo (Promega) was added to each well and incubated for 5 min at room temperature. Luciferase readings were collected by microplate reader. All readings were normalized as percentage of viable cells in the untreated control wells and the IC₅₀ values were calculated by Graphpad Prism software.

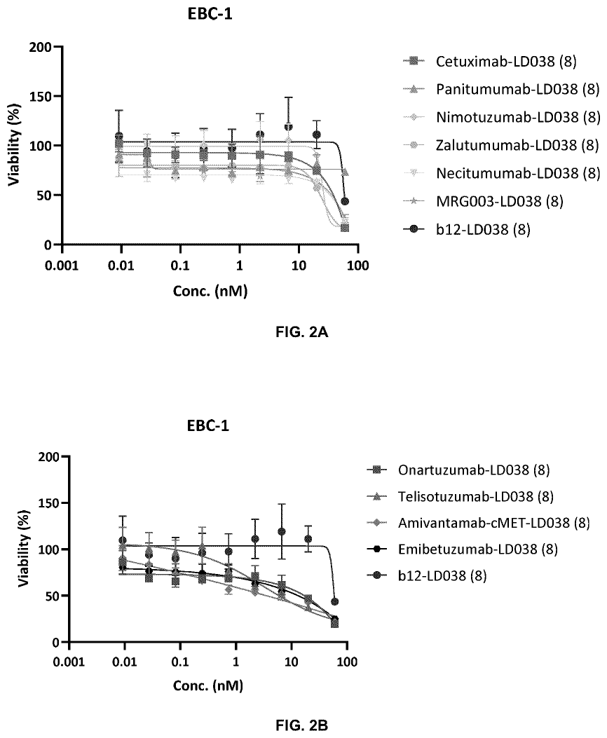

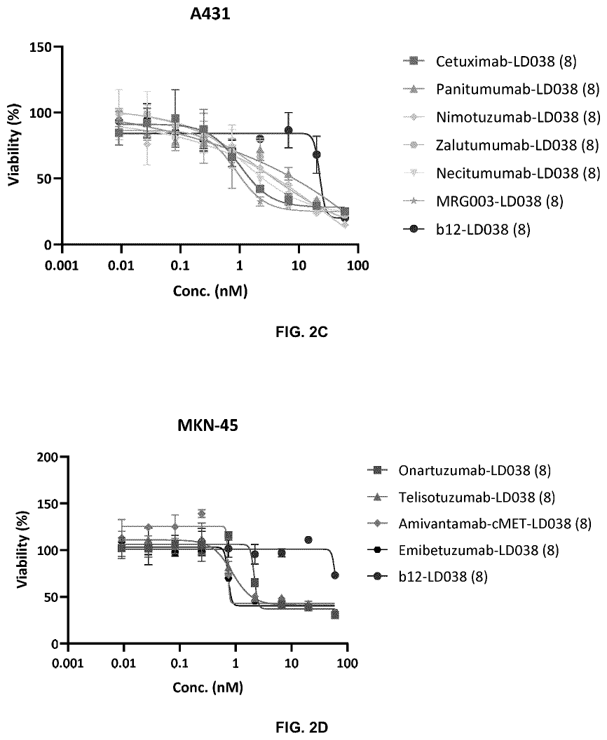

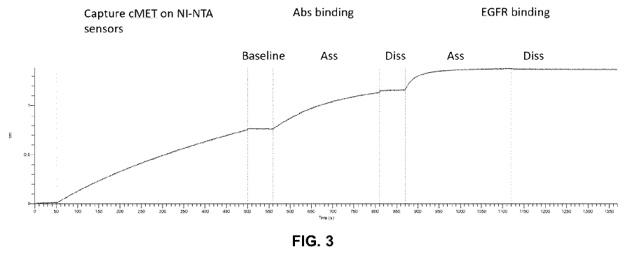

In vitro cytotoxicity of conjugates of parent mAbs and LD038 in various tumor cell lines as shown in FIGS. 2A-2D. All parent mAbs with drug conjugation showed good in vitro cytotoxicity.

### Example 10: Bispecific Format

There are a number of bispecific formats as shown in Table 6. In the Examples of the application, the scFab format (knob-in-hole) was selected, and Bsab 6, Bsab 12, Bsab 43, Bsab 44, Bsab 67, Bsab 81, Bsab 52 WT, Bsab 6 WT, Bsab 67 WT, Bsab 68 WT, and Bsab 81 WT were constructed.

TABLE 6
Format

| | |
|---|---|
| 1 + 1, scFab | Final choice |
| 1 + 1, scFab with cystine pair | Unwanting conjugation site |
| 1 + 1, Fab + scFab | No significant improvement in one step purity, CMC favours |
| | two sequence rather than three |
| 1 + 1, scFv (cystine | Unwanting conjugation site |
| bond) + scFab/Fab | |
| 1 + 1, scFv (ionic | Compromised stability, low Tonset and Tm, risky for |
| bond) + scFab/Fab | development |
| 1 + 1, scFv + scFv | Compromised stability, low Tonset and Tm, risky for |
| | development, better one step purity |
| 1 + 2, scFab + tandem scfv | Not enough biology reasoning behind biparatopic cMET arm |
| 2 + 2, IgG − scFv | Never tried due to EGFR avidity not suitable for bispecific |
| | MOA |

Special engineering

| | |
|---|---|
| 1 + 1 scFab with deaffinity of Teliso | No decrease in affnity |
| 1 + 1 scFab with deaffinity of Onar | Bmax decrease, no effect on EC₅₀ |

TABLE 7
Sequences of Bsab 6

| | | SEQ |
|---|---|---|
| Description | Amino Acid Sequence | ID NO: |
| Anti EGFR | AIQLTQSPSSLSASVGDRVTITCRASQDISSALVWYQQKPGKAPKLL | 134 |
| knob chain | IYDASSLESGVPSRFSGSESGTDFTLTISSLQPEDFATYYCQQFNSY | |
| | PLTFGGGTKVEIKRTVAAPSVFIFPPSDEQLKSGTASVVCLLNNFYP | |
| | REAKVQWKVDNALQSGNSQESVTEQDSKDSTYSLSSTLTLSKADY | |
| | EKHKVYACEVTHQGLSSPVTKSFNRGECGGGGSGGGGSGGGGS | |
| | GGGGSGGGGSGGGGSGGGGSGGGGSQVQLVESGGGVVQPGR | |
| | SLRLSCAASGFTFSTYGMHWVRQAPGKGLEWVAVIWDDGSYKYY | |
| | GDSVKGRFTISRDNSKNTLYLQMNSLRAEDTAVYYCARDGITMVR | |
| | GVMKDYFDYWGQGTLVTVSSASTKGPSVFPLAPSSKSTSGGTAAL | |
| | GCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVCTLPPSRDELTKNQVSLWCLVKG | |
| | FYPSDIAVEWESNGQPENNYKTTPPVLDSDGSFFLYSKLTVDKSR | |
| | WQQGNVFSCSVMHEALHNHYTQKSLSLSPGK | |
| Anti EGFR | QVQLVESGGGVVQPGRSLRLSCAASGFTFSTYGMHWVRQAPGKG | 135 |
| heavy chain | LEWVAVIWDDGSYKYYGDSVKGRFTISRDNSKNTLYLQMNSLRAE | |
| | DTAVYYCARDGITMVRGVMKDYFDYWGQGTLVTVSSASTKGPSVF | |
| | PLAPSSKSTSGGTAALGCLVKDYFPEPVTVSWNSGALTSGVHTFP | |
| | AVLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCVV | |
| | VDVSHEDPEVKFNWYVDGVEVHNAKTKPREEQYNSTYRVVSVLTV | |
| | LHQDWLNGKEYKCKVSNKALPAPIEKTISKAKGQPREPQVCTLPPS | |
| | RDELTKNQVSLWCLVKGFYPSDIAVEWESNGQPENNYKTTPPVLD | |
| | SDGSFFLYSKLTVDKSRWQQGNVFSCSVMHEALHNHYTQKSLSLS | |
| | PGK | |
| Anti EGFR | AIQLTQSPSSLSASVGDRVTITCRASQDISSALVWYQQKPGKAPKLL | 136 |
| light chain | IYDASSLESGVPSRFSGSESGTDFTLTISSLQPEDFATYYCQQFNSY | |
| | PLTFGGGTKVEIKRTVAAPSVFIFPPSDEQLKSGTASVVCLLNNFYP | |
| | REAKVQWKVDNALQSGNSQESVTEQDSKDSTYSLSSTLTLSKADY | |
| | EKHKVYACEVTHQGLSSPVTKSFNRGEC | |
| Anti EGFR | QVQLVESGGGVVQPGRSLRLSCAASGFTFSTYGMHWVRQAPGKG | 137 |
| VH | LEWVAVIWDDGSYKYYGDSVKGRFTISRDNSKNTLYLQMNSLRAE | |
| | DTAVYYCARDGITMVRGVMKDYFDYWGQGTLVTVSS | |
| Anti EGFR | AIQLTQSPSSLSASVGDRVTITCRASQDISSALVWYQQKPGKAPKLL | 138 |
| VL | IYDASSLESGVPSRFSGSESGTDFTLTISSLQPEDFATYYCQQFNSY | |
| | PLTFGGGTKVEIK | |
| Anti EGFR | GFTFSTYG | 139 |
| HCDR1 | | |
| Anti EGFR | IWDDGSYK | 140 |
| HCDR2 | | |
| Anti EGFR | ARDGITMVRGVMKDYFDY | 141 |
| HCDR3 | | |
| Anti EGFR | QDISSA | 142 |
| LCDR1 | | |
| Anti EGFR | DAS | |
| LCDR2 | | |
| Anti EGFR | QQFNSYPLT | 143 |
| LCDR3 | | |
| Anti cMET | DIVMTQSPDSLAVSLGERATINCKSSESVDSYANSFLHWYQQKPG | 144 |
| hole chain | QPPKLLIYRASTRESGVPDRFSGSGSGTDFTLTISSLQAEDVAVYYC | |
| | QQSKEDPLTFGGGTKVEIKRTVAAPSVFIFPPSDEQLKSGTASVVCL | |
| | LNNFYPREAKVQWKVDNALQSGNSQESVTEQDSKDSTYSLSSTLT | |
| | LSKADYEKHKVYACEVTHQGLSSPVTKSFNRGECGGGGSGGGGS | |
| | GGGGSGGGGSGGGGSGGGGSGGGGSGGGGSQVQLVQSGAEV | |
| | KKPGASVKVSCKASGYIFTAYTMHWVRQAPGQGLEWMGWIKPNN | |
| | GLANYAQKFQGRVTMTRDTSISTAYMELSRLRSDDTAVYYCARSEI | |
| | TTEFDYWGQGTLVTVSSASTKGPSVFPLAPSSKSTSGGTAALGCL | |
| | VKDYFPEPVTVSWNSGALTSGVHTFPAVLQSSGLYSLSSVVTVPSS | |
| | SLGTQTYICNVNHKPSNTKVDKKVEPKSCDKTHTCPPCPAPEAAG | |
| | GPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKA | |
| | LPAPIEKTISKAKGQPREPQVYTLPPCRDELTKNQVSLSCAVKGFYP | |
| | SDIAVEWESNGQPENNYKTTPPVLDSDGSFFLVSKLTVDKSRWQQ | |
| | GNVFSCSVMHEALHNHYTQKSLSLSPGK | |
| Anti cMET | QVQLVQSGAEVKKPGASVKVSCKASGYIFTAYTMHWVRQAPGQG | 145 |
| heavy chain | LEWMGWIKPNNGLANYAQKFQGRVTMTRDTSISTAYMELSRLRSD | |
| | DTAVYYCARSEITTEFDYWGQGTLVTVSSASTKGPSVFPLAPSSKS | |
| | TSGGTAALGCLVKDYFPEPVTVSWNSGALTSGVHTFPAVLQSSGL | |
| | YSLSSVVTVPSSSLGTQTYICNVNHKPSNTKVDKKVEPKSCDKTHT | |
| | CPPCPAPEAAGGPSVFLFPPKPKDTLMISRTPEVTCVVVDVSHEDP | |
| | EVKFNWYVDGVEVHNAKTKPREEQYNSTYRVVSVLTVLHQDWLN | |
| | GKEYKCKVSNKALPAPIEKTISKAKGQPREPQVYTLPPCRDELTKN | |
| | QVSLSCAVKGFYPSDIAVEWESNGQPENNYKTTPPVLDSDGSFFL | |
| | VSKLTVDKSRWQQGNVFSCSVMHEALHNHYTQKSLSLSPGK | |
| Anti cMET | DIVMTQSPDSLAVSLGERATINCKSSESVDSYANSFLHWYQQKPG | 146 |
| light chain | QPPKLLIYRASTRESGVPDRFSGSGSGTDFTLTISSLQAEDVAVYYC | |
| | QQSKEDPLTFGGGTKVEIKRTVAAPSVFIFPPSDEQLKSGTASVVCL | |
| | LNNFYPREAKVQWKVDNALQSGNSQESVTEQDSKDSTYSLSSTLT | |
| | LSKADYEKHKVYACEVTHQGLSSPVTKSFNRGEC | |
| Anti cMET | QVQLVQSGAEVKKPGASVKVSCKASGYIFTAYTMHWVRQAPGQG | 147 |
| VH | LEWMGWIKPNNGLANYAQKFQGRVTMTRDTSISTAYMELSRLRSD | |
| | DTAVYYCARSEITTEFDYWGQGTLVTVSS | |
| Anti cMET | DIVMTQSPDSLAVSLGERATINCKSSESVDSYANSFLHWYQQKPG | 148 |
| VL | QPPKLLIYRASTRESGVPDRFSGSGSGTDFTLTISSLQAEDVAVYYC | |
| | QQSKEDPLTFGGGTKVEIK | |
| Anti cMET | GYIFTAYT | 149 |
| HCDR1 | | |
| Anti cMET | IKPNNGLA | 150 |
| HCDR2 | | |
| Anti cMET | ARSEITTEFDY | 151 |
| HCDR3 | | |
| Anti cMET | ESVDSYANSF | 152 |
| LCDR1 | | |
| Anti cMET | RAS | |
| LCDR2 | | |
| Anti cMET | QQSKEDPLT | 153 |
| LCDR3 | | |

TABLE 8
Sequences of Bsab 12

| | | SEQ |
|---|---|---|
| Description | Amino Acid Sequence | ID NO: |
| Anti EGFR | AIQLTQSPSSLSASVGDRVTITCRASQDISSALVWYQQKPGKAPKLL | 154 |
| knob chain | IYDASSLESGVPSRFSGSESGTDFTLTISSLQPEDFATYYCQQFNSY | |
| | PLTFGGGTKVEIKRTVAAPSVFIFPPSDEQLKSGTASVVCLLNNFYP | |
| | REAKVQWKVDNALQSGNSQESVTEQDSKDSTYSLSSTLTLSKADY | |
| | EKHKVYACEVTHQGLSSPVTKSFNRGECGGGGSGGGGSGGGGS | |
| | GGGGSGGGGSGGGGSGGGGSGGGGSQVQLVESGGGVVQPGR | |
| | SLRLSCAASGFTFSTYGMHWVRQAPGKGLEWVAVIWDDGSYKYY | |
| | GDSVKGRFTISRDNSKNTLYLQMNSLRAEDTAVYYCARDGITMVR | |
| | GVMKDYFDYWGQGTLVTVSSASTKGPSVFPLAPSSKSTSGGTAAL | |
| | GCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVCTLPPSRDELTKNQVSLWCLVKG | |
| | FYPSDIAVEWESNGQPENNYKTTPPVLDSDGSFFLYSKLTVDKSR | |
| | WQQGNVFSCSVMHEALHNHYTQKSLSLSPGK | |
| Anti EGFR | QVQLVESGGGVVQPGRSLRLSCAASGFTFSTYGMHWVRQAPGKG | 155 |
| heavy chain | LEWVAVIWDDGSYKYYGDSVKGRFTISRDNSKNTLYLQMNSLRAE | |
| | DTAVYYCARDGITMVRGVMKDYFDYWGQGTLVTVSSASTKGPSVF | |
| | PLAPSSKSTSGGTAALGCLVKDYFPEPVTVSWNSGALTSGVHTFP | |
| | AVLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCVV | |
| | VDVSHEDPEVKFNWYVDGVEVHNAKTKPREEQYNSTYRVVSVLTV | |
| | LHQDWLNGKEYKCKVSNKALPAPIEKTISKAKGQPREPQVCTLPPS | |
| | RDELTKNQVSLWCLVKGFYPSDIAVEWESNGQPENNYKTTPPVLD | |
| | SDGSFFLYSKLTVDKSRWQQGNVFSCSVMHEALHNHYTQKSLSLS | |
| | PGK | |
| Anti EGFR | AIQLTQSPSSLSASVGDRVTITCRASQDISSALVWYQQKPGKAPKLL | 156 |
| light chain | IYDASSLESGVPSRFSGSESGTDFTLTISSLQPEDFATYYCQQFNSY | |
| | PLTFGGGTKVEIKRTVAAPSVFIFPPSDEQLKSGTASVVCLLNNFYP | |
| | REAKVQWKVDNALQSGNSQESVTEQDSKDSTYSLSSTLTLSKADY | |
| | EKHKVYACEVTHQGLSSPVTKSFNRGEC | |
| Anti EGFR | QVQLVESGGGVVQPGRSLRLSCAASGFTFSTYGMHWVRQAPGKG | 157 |
| VH | LEWVAVIWDDGSYKYYGDSVKGRFTISRDNSKNTLYLQMNSLRAE | |
| | DTAVYYCARDGITMVRGVMKDYFDYWGQGTLVTVSS | |
| Anti EGFR | AIQLTQSPSSLSASVGDRVTITCRASQDISSALVWYQQKPGKAPKLL | 158 |
| VL | IYDASSLESGVPSRFSGSESGTDFTLTISSLQPEDFATYYCQQFNSY | |
| | PLTFGGGTKVEIK | |
| Anti EGFR | GFTFSTYG | 139 |
| HCDR1 | | |
| Anti EGFR | IWDDGSYK | 140 |
| HCDR2 | | |
| Anti EGFR | ARDGITMVRGVMKDYFDY | 141 |
| HCDR3 | | |
| Anti EGFR | QDISSA | 142 |
| LCDR1 | | |
| Anti EGFR | DAS | |
| LCDR2 | | |
| Anti EGFR | QQFNSYPLT | 143 |
| LCDR3 | | |
| Anti cMET | DIQMTQSPSSLSASVGDRVTITCKSSQSLLYTSSQKNYLAWYQQKP | 159 |
| hole chain | GKAPKLLIYWASTRESGVPSRFSGSGSGTDFTLTISSLQPEDFATYY | |
| | CQQYYAYPWTFGQGTKVEIKRTVAAPSVFIFPPSDEQLKSGTASVV | |
| | CLLNNFYPREAKVQWKVDNALQSGNSQESVTEQDSKDSTYSLSST | |
| | LTLSKADYEKHKVYACEVTHQGLSSPVTKSFNRGECGGGGSGGG | |
| | GSGGGGSGGGGSGGGGSGGGGSGGGGSGGGGSEVQLVESGG | |
| | GLVQPGGSLRLSCAASGYTFTSYWLHWVRQAPGKGLEWVGMIDP | |
| | SNSDTRFNPNFKDRFTISADTSKNTAYLQMNSLRAEDTAVYYCATY | |
| | RSYVTPLDYWGQGTLVTVSSASTKGPSVFPLAPSSKSTSGGTAAL | |
| | GCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVYTLPPCREEMTKNQVSLSCAVKG | |
| | FYPSDIAVEWESNGQPENNYKTTPPVLDSDGSFFLVSKLTVDKSR | |
| | WQQGNVFSCSVMHEALHNHYTQKSLSLSPGK | |
| Anti cMET | EVQLVESGGGLVQPGGSLRLSCAASGYTFTSYWLHWVRQAPGKG | 160 |
| heavy chain | LEWVGMIDPSNSDTRFNPNFKDRFTISADTSKNTAYLQMNSLRAED | |
| | TAVYYCATYRSYVTPLDYWGQGTLVTVSSASTKGPSVFPLAPSSKS | |
| | TSGGTAALGCLVKDYFPEPVTVSWNSGALTSGVHTFPAVLQSSGL | |
| | YSLSSWTVPSSSLGTQTYICNVNHKPSNTKVDKKVEPKSCDKTHT | |
| | CPPCPAPEAAGGPSVFLFPPKPKDTLMISRTPEVTCVVVDVSHEDP | |
| | EVKFNWYVDGVEVHNAKTKPREEQYNSTYRVVSVLTVLHQDWLN | |
| | GKEYKCKVSNKALPAPIEKTISKAKGQPREPQVYTLPPCREEMTKN | |
| | QVSLSCAVKGFYPSDIAVEWESNGQPENNYKTTPPVLDSDGSFFL | |
| | VSKLTVDKSRWQQGNVFSCSVMHEALHNHYTQKSLSLSPGK | |
| Anti cMET | DIQMTQSPSSLSASVGDRVTITCKSSQSLLYTSSQKNYLAWYQQKP | 161 |
| light chain | GKAPKLLIYWASTRESGVPSRFSGSGSGTDFTLTISSLQPEDFATYY | |
| | CQQYYAYPWTFGQGTKVEIKRTVAAPSVFIFPPSDEQLKSGTASVV | |
| | CLLNNFYPREAKVQWKVDNALQSGNSQESVTEQDSKDSTYSLSST | |
| | LTLSKADYEKHKVYACEVTHQGLSSPVTKSFNRGEC | |
| Anti cMET | EVQLVESGGGLVQPGGSLRLSCAASGYTFTSYWLHWVRQAPGKG | 162 |
| VH | LEWVGMIDPSNSDTRFNPNFKDRFTISADTSKNTAYLQMNSLRAED | |
| | TAVYYCATYRSYVTPLDYWGQGTLVTVSS | |
| Anti cMET | DIQMTQSPSSLSASVGDRVTITCKSSQSLLYTSSQKNYLAWYQQKP | 163 |
| VL | GKAPKLLIYWASTRESGVPSRFSGSGSGTDFTLTISSLQPEDFATYY | |
| | CQQYYAYPWTFGQGTKVEIK | |
| Anti cMET | GYTFTSYWLH | 164 |
| HCDR1 | | |
| Anti cMET | IDPSNSDT | 165 |
| HCDR2 | | |
| Anti cMET | ATYRSYVTPLDY | 166 |
| HCDR3 | | |
| Anti cMET | QSLLYTSSQKNY | 167 |
| LCDR1 | | |
| Anti cMET | WAS | |
| LCDR2 | | |
| Anti cMET | QQYYAYPWT | 168 |
| LCDR3 | | |

TABLE 9
Sequences of Bsab 43

| | | SEQ ID |
|---|---|---|
| Description | Amino Acid Sequence | NO: |
| Anti EGFR | DIQMTQSPSSLSASVGDRVTITCRSSQNIVHSNGNTYLDWYQQTPG | 169 |
| knob chain | KAPKLLIYKVSNRFSGVPSRFSGSGSGTDFTFTISSLQPEDIATYY | |
| | CFQYSHVPWTFGQGTKLQITRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGECGGGGSGG | |
| | GGSGGGGSGGGGSGGGGSGGGGSGGGGSGGGGSQVQLQQS | |
| | GAEVKKPGSSVKVSCKASGYTFTNYYIYWVRQAPGQGLEWIGGIN | |
| | PTSGGSNFNEKFKTRVTITADESSTTAYMELSSLRSEDTAFYFCTR | |
| | QGLWFDSDGRGFDFWGQGTTVTVSSASTKGPSVFPLAPSSKSTS | |
| | GGTAALGCLVKDYFPEPVTVSWNSGALTSGVHTFPAVLQSSGLYS | |
| | LSSWVTVPSSSLGTQTYICNVNHKPSNTKVDKKVEPKSCDKTHTCP | |
| | PCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVCTLPPSRDELTKNQV | |
| | SLWCLVKGFYPSDIAVEWESNGQPENNYKTTPPVLDSDGSFFLYS | |
| | KLTVDKSRWQQGNVFSCSVMHEALHNHYTQKSLSLSPGK | |
| Anti EGFR | QVQLQQSGAEVKKPGSSVKVSCKASGYTFTNYYIYWVRQAPGQG | 170 |
| heavy chain | LEWIGGINPTSGGSNFNEKFKTRVTITADESSTTAYMELSSLRSED | |
| | TAFYFCTRQGLWFDSDGRGFDFWGQGTTVTVSSASTKGPSVFPL | |
| | APSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKS | |
| | CDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVL | |
| | HQDWLNGKEYKCKVSNKALPAPIEKTISKAKGQPREPQVCTLPPS | |
| | RDELTKNQVSLWCLVKGFYPSDIAVEWESNGQPENNYKTTPPVLD | |
| | SDGSFFLYSKLTVDKSRWQQGNVFSCSVMHEALHNHYTQKSLSL | |
| | SPGK | |
| Anti EGFR | DIQMTQSPSSLSASVGDRVTITCRSSQNIVHSNGNTYLDWYQQTPG | 171 |
| light chain | KAPKLLIYKVSNRFSGVPSRFSGSGSGTDFTFTISSLQPEDIATYY | |
| | CFQYSHVPWTFGQGTKLQITRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGEC | |
| Anti EGFR | QVQLQQSGAEVKKPGSSVKVSCKASGYTFTNYYIYWVRQAPGQGL | 172 |
| VH | EWIGGINPTSGGSNFNEKFKTRVTITADESSTTAYMELSSLRSED | |
| | TAFYFCTRQGLWFDSDGRGFDFWGQGTTVTVSS | |
| Anti EGFR | DIQMTQSPSSLSASVGDRVTITCRSSQNIVHSNGNTYLDWYQQTP | 173 |
| VL | GKAPKLLIYKVSNRFSGVPSRFSGSGSGTDFTFTISSLQPEDIAT | |
| | YYCFQYSHVPWTFGQGTKLQIT | |
| Anti EGFR | GYTFTNYY | 174 |
| HCDR1 | | |
| Anti EGFR | INPTSGGS | 175 |
| HCDR2 | | |
| Anti EGFR | TRQGLWFDSDGRGFDF | 176 |
| HCDR3 | | |
| Anti EGFR | QNIVHSNGNTY | 177 |
| LCDR1 | | |
| Anti EGFR | KVS | |
| LCDR2 | | |
| Anti EGFR | FQYSHVPWT | 178 |
| LCDR3 | | |
| Anti cMET | DIVMTQSPDSLAVSLGERATINCKSSESVDSYANSFLHWYQQKPG | 179 |
| hole chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGECGGGGSGG | |
| | GGSGGGGGGGGSGGGGSGGGGSGGGGGGGGSQVQLVQSG | |
| | AEVKKPGASVKVSCKASGYIFTAYTMHWVRQAPGQGLEWMGWIK | |
| | PNNGLANYAQKFQGRVTMTRDTSISTAYMELSRLRSDDTAVYYCA | |
| | RSEITTEFDYWGQGTLVTVSSASTKGPSVFPLAPSSKSTSGGTAAL | |
| | GCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAP | |
| | EAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAKGQPREPQVYTLPPCRDELTKNQVSLSC | |
| | AVKGFYPSDIAVEWESNGQPENNYKTTPPVLDSDGSFFLVSKLTV | |
| | DKSRWQQGNVFSCSVMHEALHNHYTQKSLSLSPGK | |
| Anti cMET | QVQLVQSGAEVKKPGASVKVSCKASGYIFTAYTMHWVRQAPGQG | 180 |
| heavy chain | LEWMGWIKPNNGLANYAQKFQGRVTMTRDTSISTAYMELSRLRS | |
| | DDTAVYYCARSEITTEFDYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDKT | |
| | HTCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDW | |
| | LNGKEYKCKVSNKALPAPIEKTISKAKGQPREPQVYTLPPCRDELT | |
| | KNQVSLSCAVKGFYPSDIAVEWESNGQPENNYKTTPPVLDSDGSF | |
| | FLVSKLTVDKSRWQQGNVFSCSVMHEALHNHYTQKSLSLSPGK | |
| Anti cMET | DIVMTQSPDSLAVSLGERATINCKSSESVDSYANSFLHWYQQKPG | 181 |
| light chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGEC | |
| Anti cMET | QVQLVQSGAEVKKPGASVKVSCKASGYIFTAYTMHWVRQAPGQG | 182 |
| VH | LEWMGWIKPNNGLANYAQKFQGRVTMTRDTSISTAYMELSRLRS | |
| | DDTAVYYCARSEITTEFDYWGQGTLVTVSS | |
| Anti cMET | DIVMTQSPDSLAVSLGERATINCKSSESVDSYANSFLHWYQQKPG | 183 |
| VL | QPPKLLIYRASTRESGVPDRFSGSGSGTDFTLTISSLQAEDVAVYY | |
| | CQQSKEDPLTFGGGTKVEIK | |
| Anti cMET | GYIFTAYT | 149 |
| HCDR1 | | |
| Anti cMET | IKPNNGLA | 150 |
| HCDR2 | | |
| Anti cMET | ARSEITTEFDY | 151 |
| HCDR3 | | |
| Anti cMET | ESVDSYANSF | 152 |
| LCDR1 | | |
| Anti cMET | RAS | |
| LCDR2 | | |
| Anti cMET | QQSKEDPLT | 153 |
| LCDR3 | | |

TABLE 10
Sequences of Bsab 44

| | | SEQ ID |
|---|---|---|
| Description | Amino Acid Sequence | NO: |
| Anti EGFR | DIQMTQSPSSLSASVGDRVTITCRSSQNIVHSNGNTYLDWYQQTP | 184 |
| knob chain | GKAPKLLIYKVSNRFSGVPSRFSGSGSGTDFTFTISSLQPEDIATYY | |
| | CFQYSHVPWTFGQGTKLQITRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGECGGGGSGG | |
| | GGSGGGGSGGGGGGGGSGGGGSGGGGSGGGGSQVQLQQS | |
| | GAEVKKPGSSVKVSCKASGYTFTNYYIYWVRQAPGQGLEWIGGIN | |
| | PTSGGSNFNEKFKTRVTITADESSTTAYMELSSLRSEDTAFYFCTR | |
| | QGLWFDSDGRGFDFWGQGTTVTVSSASTKGPSVFPLAPSSKSTS | |
| | GGTAALGCLVKDYFPEPVTVSWNSGALTSGVHTFPAVLQSSGLYS | |
| | LSSWVTVPSSSLGTQTYICNVNHKPSNTKVDKKVEPKSCDKTHTCP | |
| | PCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVCTLPPSRDELTKNQV | |
| | SLWCLVKGFYPSDIAVEWESNGQPENNYKTTPPVLDSDGSFFLYS | |
| | KLTVDKSRWQQGNVFSCSVMHEALHNHYTQKSLSLSPGK | |
| Anti EGFR | QVQLQQSGAEVKKPGSSVKVSCKASGYTFTNYYIYWVRQAPGQG | 185 |
| heavy chain | LEWIGGINPTSGGSNFNEKFKTRVTITADESSTTAYMELSSLRSED | |
| | TAFYFCTRQGLWFDSDGRGFDFWGQGTTVTVSSASTKGPSVFPL | |
| | APSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKS | |
| | CDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVL | |
| | HQDWLNGKEYKCKVSNKALPAPIEKTISKAKGQPREPQVCTLPPS | |
| | RDELTKNQVSLWCLVKGFYPSDIAVEWESNGQPENNYKTTPPVLD | |
| | SDGSFFLYSKLTVDKSRWQQGNVFSCSVMHEALHNHYTQKSLSL | |
| | SPGK | |
| Anti EGFR | DIQMTQSPSSLSASVGDRVTITCRSSQNIVHSNGNTYLDWYQQTP | 186 |
| light chain | GKAPKLLIYKVSNRFSGVPSRFSGSGSGTDFTFTISSLQPEDIATYY | |
| | CFQYSHVPWTFGQGTKLQITRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGEC | |
| Anti EGFR | QVQLQQSGAEVKKPGSSVKVSCKASGYTFTNYYIYWVRQAPGQG | 187 |
| VH | LEWIGGINPTSGGSNFNEKFKTRVTITADESSTTAYMELSSLRSED | |
| | TAFYFCTRQGLWFDSDGRGFDFWGQGTTVTVSS | |
| Anti EGFR | DIQMTQSPSSLSASVGDRVTITCRSSQNIVHSNGNTYLDWYQQTP | 188 |
| VL | GKAPKLLIYKVSNRFSGVPSRFSGSGSGTDFTFTISSLQPEDIAT | |
| | YYCFQYSHVPWTFGQGTKLQIT | |
| Anti EGFR | GYTFTNYY | 174 |
| HCDR1 | | |
| Anti EGFR | INPTSGGS | 175 |
| HCDR2 | | |
| Anti EGFR | TRQGLWFDSDGRGFDF | 176 |
| HCDR3 | | |
| Anti EGFR | QNIVHSNGNTY | 177 |
| LCDR1 | | |
| Anti EGFR | KVS | |
| LCDR2 | | |
| Anti EGFR | FQYSHVPWT | 178 |
| LCDR3 | | |
| Anti cMET | DIQMTQSPSSLSASVGDRVTITCKSSQSLLYTSSQKNYLAWYQQK | 189 |
| hole chain | PGKAPKLLIYWASTRESGVPSRFSGSGSGTDFTLTISSLQPEDFAT | |
| | YYCQQYYAYPWTFGQGTKVEIKRTVAAPSVFIFPPSDEQLKSGTA | |
| | SWVCLLNNFYPREAKVQWKVDNALQSGNSQESVTEQDSKDSTYS | |
| | LSSTLTLSKADYEKHKVYACEVTHQGLSSPVTKSFNRGECGGGGS | |
| | GGGGSGGGGGGGGSGGGGSGGGGSGGGGSGGGGSEVQLVE | |
| | SGGGLVQPGGSLRLSCAASGYTFTSYWLHWVRQAPGKCLEWVG | |
| | MIDPSNSDTRFNPNFKDRFTISADTSKNTAYLQMNSLRAEDTAVYY | |
| | CATYRSYVTPLDYWGQGTLVTVSSASTKGPSVFPLAPSSKSTSGG | |
| | TAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPP | |
| | CPAPEAAGGPSVFLFPPKPKDTLMISRTPEVTCVVVDVSHEDPEVK | |
| | FNWYVDGVEVHNAKTKPREEQYNSTYRVVSVLTVLHQDWLNGKE | |
| | YKCKVSNKALPAPIEKTISKAKGQPREPQVYTLPPCREEMTKNQVS | |
| | LSCAVKGFYPSDIAVEWESNGQPENNYKTTPPVLDSDGSFFLVSK | |
| | LTVDKSRWQQGNVFSCSVMHEALHNHYTQKSLSLSPGK | |
| Anti cMET | EVQLVESGGGLVQPGGSLRLSCAASGYTFTSYWLHWVRQAPGKC | 190 |
| heavy chain | LEWVGMIDPSNSDTRFNPNFKDRFTISADTSKNTAYLQMNSLRAE | |
| | DTAVYYCATYRSYVTPLDYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDKT | |
| | HTCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDW | |
| | LNGKEYKCKVSNKALPAPIEKTISKAKGQPREPQVYTLPPCREEMT | |
| | KNQVSLSCAVKGFYPSDIAVEWESNGQPENNYKTTPPVLDSDGSF | |
| | FLVSKLTVDKSRWQQGNVFSCSVMHEALHNHYTQKSLSLSPGK | |
| Anti cMET | DIQMTQSPSSLSASVGDRVTITCKSSQSLLYTSSQKNYLAWYQQK | 191 |
| light chain | PGKAPKLLIYWASTRESGVPSRFSGSGSGTDFTLTISSLQPEDFAT | |
| | YYCQQYYAYPWTFGQGTKVEIKRTVAAPSVFIFPPSDEQLKSGTA | |
| | SWVCLLNNFYPREAKVQWKVDNALQSGNSQESVTEQDSKDSTYS | |
| | LSSTLTLSKADYEKHKVYACEVTHQGLSSPVTKSFNRGEC | |
| Anti cMET | EVQLVESGGGLVQPGGSLRLSCAASGYTFTSYWLHWVRQAPGKC | 192 |
| VH | LEWVGMIDPSNSDTRFNPNFKDRFTISADTSKNTAYLQMNSLRAE | |
| | DTAVYYCATYRSYVTPLDYWGQGTLVTVSS | |
| Anti cMET | DIQMTQSPSSLSASVGDRVTITCKSSQSLLYTSSQKNYLAWYQQK | 193 |
| VL | PGKAPKLLIYWASTRESGVPSRFSGSGSGTDFTLTISSLQPEDFAT | |
| | YYCQQYYAYPWTFGQGTKVEIK | |
| Anti cMET | GYTFTSYW | 194 |
| HCDR1 | | |
| Anti cMET | IDPSNSDT | 165 |
| HCDR2 | | |
| Anti cMET | ATYRSYVTPLDY | 166 |
| HCDR3 | | |
| Anti cMET | QSLLYTSSQKNY | 167 |
| LCDR1 | | |
| Anti cMET | WAS | |
| LCDR2 | | |
| Anti cMET | QQYYAYPWT | 168 |
| LCDR3 | | |

TABLE 11
Sequences of Bsab 67

| | | SEQ ID |
|---|---|---|
| Description | Amino Acid Sequence | NO: |
| Anti EGFR | AIQLTQSPESLSASVGDRVTITCRASQDISSALVWYQQKPGKAPKL | 195 |
| knob chain | LIYDASSLESGVPDRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGECGGGGSGGGGSGGG | |
| | GSGGGGSGGGGSGGGGSGGGGSGGGGSQVQLVESGGGVVQP | |
| | GRSLRLSCAASGFTFSTYGMHWVRQAPGQGLEWVAVIWDDGSY | |
| | KYYGDSVKGRFTISRDNSKNTLYLQMNSLRAEDTAVYYCARDGIT | |
| | MVRGVMKDYFDYWGQGTLVTVSSASTKGPSVFPLAPSSKSTSGG | |
| | TAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPP | |
| | CPAPEAAGGPSVFLFPPKPKDTLMISRTPEVTCVVVDVSHEDPEVK | |
| | FNWYVDGVEVHNAKTKPREEQYNSTYRVVSVLTVLHQDWLNGKE | |
| | YKCKVSNKALPAPIEKTISKAKGQPREPQVCTLPPSRDELTKNQVS | |
| | LWCLVKGFYPSDIAVEWESNGQPENNYKTTPPVLDSDGSFFLYSK | |
| | LTVDKSRWQQGNVFSCSVMHEALHNHYTQKSLSLSPGK | |
| Anti EGFR | QVQLVESGGGVVQPGRSLRLSCAASGFTFSTYGMHWVRQAPGQ | 196 |
| heavy chain | GLEWVAVIWDDGSYKYYGDSVKGRFTISRDNSKNTLYLQMNSLRA | |
| | EDTAVYYCARDGITMVRGVMKDYFDYWGQGTLVTVSSASTKGPS | |
| | VFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKV | |
| | EPKSCDKTHTCPPCPAPEAAGGPSVFLFPPKPKDTLMISRTPEVTC | |
| | WVDVSHEDPEVKFNWYVDGVEVHNAKTKPREEQYNSTYRVVSV | |
| | LTVLHQDWLNGKEYKCKVSNKALPAPIEKTISKAKGQPREPQVCTL | |
| | PPSRDELTKNQVSLWCLVKGFYPSDIAVEWESNGQPENNYKTTPP | |
| | VLDSDGSFFLYSKLTVDKSRWQQGNVFSCSVMHEALHNHYTQKS | |
| | LSLSPGK | |
| Anti EGFR | AIQLTQSPESLSASVGDRVTITCRASQDISSALVWYQQKPGKAPKL | 197 |
| light chain | LIYDASSLESGVPDRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| Anti EGFR | QVQLVESGGGVVQPGRSLRLSCAASGFTFSTYGMHWVRQAPGQ | 198 |
| VH | GLEWVAVIWDDGSYKYYGDSVKGRFTISRDNSKNTLYLQMNSLRA | |
| | EDTAVYYCARDGITMVRGVMKDYFDYWGQGTLVTVSS | |
| Anti EGFR | AIQLTQSPESLSASVGDRVTITCRASQDISSALVWYQQKPGKAPKL | 199 |
| VL | LIYDASSLESGVPDRFSGSESGTDFTLTISSLQPEDFATYYCQQFN | |
| | SYPLTFGGGTKVEIK | |
| Anti EGFR | GFTFSTYG | 139 |
| HCDR1 | | |
| Anti EGFR | IWDDGSYK | 140 |
| HCDR2 | | |
| Anti EGFR | ARDGITMVRGVMKDYFDY | 141 |
| HCDR3 | | |
| Anti EGFR | QDISSA | 142 |
| LCDR1 | | |
| Anti EGFR | DAS | |
| LCDR2 | | |
| Anti EGFR | QQFNSYPLT | 143 |
| LCDR3 | | |
| Anti cMET | DIVMTQSPDSLAVSLGERATINCKSSESVDSYANSFLHWYQQKPG | 200 |
| hole chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGECGGGGSGG | |
| | GGSGGGGSGGGGSGGGGSGGGGSGGGGGGGGSQVQLVQSG | |
| | AEVKKPGASVKVSCKASGYIFTAYTMHWVRQAPGQGLEWMGWIK | |
| | PNNGLANYAQKFQGRVTMTRDTSISTAYMELSRLRSDDTAVYYCA | |
| | RSEITTEFDYWGQGTLVTVSSASTKGPSVFPLAPSSKSTSGGTAAL | |
| | GCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAP | |
| | EAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAKGQPREPQVYTLPPCRDELTKNQVSLSC | |
| | AVKGFYPSDIAVEWESNGQPENNYKTTPPVLDSDGSFFLVSKLTV | |
| | DKSRWQQGNVFSCSVMHEALHNHYTQKSLSLSPGK | |
| Anti cMET | QVQLVQSGAEVKKPGASVKVSCKASGYIFTAYTMHWVRQAPGQG | 201 |
| heavy chain | LEWMGWIKPNNGLANYAQKFQGRVTMTRDTSISTAYMELSRLRS | |
| | DDTAVYYCARSEITTEFDYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDKT | |
| | HTCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDW | |
| | LNGKEYKCKVSNKALPAPIEKTISKAKGQPREPQVYTLPPCRDELT | |
| | KNQVSLSCAVKGFYPSDIAVEWESNGQPENNYKTTPPVLDSDGSF | |
| | FLVSKLTVDKSRWQQGNVFSCSVMHEALHNHYTQKSLSLSPGK | |
| Anti cMET | DIVMTQSPDSLAVSLGERATINCKSSESVDSYANSFLHWYQQKPG | 202 |
| light chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGEC | |
| Anti cMET | QVQLVQSGAEVKKPGASVKVSCKASGYIFTAYTMHWVRQAPGQG | 203 |
| VH | LEWMGWIKPNNGLANYAQKFQGRVTMTRDTSISTAYMELSRLRS | |
| | DDTAVYYCARSEITTEFDYWGQGTLVTVSS | |
| Anti cMET | DIVMTQSPDSLAVSLGERATINCKSSESVDSYANSFLHWYQQKPG | 204 |
| VL | QPPKLLIYRASTRESGVPDRFSGSGSGTDFTLTISSLQAEDVAVYY | |
| | CQQSKEDPLTFGGGTKVEIK | |
| Anti cMET | GYIFTAYT | 149 |
| HCDR1 | | |
| Anti cMET | IKPNNGLA | 150 |
| HCDR2 | | |
| Anti cMET | ARSEITTEFDY | 151 |
| HCDR3 | | |
| Anti cMET | ESVDSYANSF | 152 |
| LCDR1 | | |
| Anti cMET | RAS | |
| LCDR2 | | |
| Anti cMET | QQSKEDPLT | 153 |
| LCDR3 | | |

TABLE 12
Sequences of Bsab 81

| | | SEQ ID |
|---|---|---|
| Description | Amino Acid Sequence | NO: |
| Anti EGFR | AIQLTQSPESLSASVGDRVTITCRASQDISSALVWYQQKPGKAPKL | 205 |
| knob chain | LIYDASSLESGVPDRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGECGGGGSGGGGSGGG | |
| | GSGGGGSGGGGSGGGGSGGGGSGGGGSQVQLVESGGGVVQP | |
| | GRSLRLSCAASGFTFSTYGMHWVRQAPGEGLEWVAVIWDDGSYK | |
| | YYGDSVKGRFTISRDNSKNTLYLQMNSLRAEDTAVYYCARDGITM | |
| | VRGVMKDYFDYWGQGTLVTVSSASTKGPSVFPLAPSSKSTSGGT | |
| | AALGCLVKDYFPEPVTVSWNSGALTSGVHTFPAVLQSSGLYSLSS | |
| | VTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKF | |
| | NWYVDGVEVHNAKTKPREEQYNSTYRVVSVLTVLHQDWLNGKEY | |
| | KCKVSNKALPAPIEKTISKAKGQPREPQVCTLPPSRDELTKNQVSL | |
| | WCLVKGFYPSDIAVEWESNGQPENNYKTTPPVLDSDGSFFLYSKL | |
| | TVDKSRWQQGNVFSCSVMHEALHNHYTQKSLSLSPGK | |
| Anti EGFR | QVQLVESGGGVVQPGRSLRLSCAASGFTFSTYGMHWVRQAPGE | 206 |
| heavy chain | GLEWVAVIWDDGSYKYYGDSVKGRFTISRDNSKNTLYLQMNSLRA | |
| | EDTAVYYCARDGITMVRGVMKDYFDYWGQGTLVTVSSASTKGPS | |
| | VFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKV | |
| | EPKSCDKTHTCPPCPAPEAAGGPSVFLFPPKPKDTLMISRTPEVTC | |
| | WVDVSHEDPEVKFNWYVDGVEVHNAKTKPREEQYNSTYRVVSV | |
| | LTVLHQDWLNGKEYKCKVSNKALPAPIEKTISKAKGQPREPQVCTL | |
| | PPSRDELTKNQVSLWCLVKGFYPSDIAVEWESNGQPENNYKTTPP | |
| | VLDSDGSFFLYSKLTVDKSRWQQGNVFSCSVMHEALHNHYTQKS | |
| | LSLSPGK | |
| Anti EGFR | AIQLTQSPESLSASVGDRVTITCRASQDISSALVWYQQKPGKAPKL | 207 |
| light chain | LIYDASSLESGVPDRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| Anti EGFR | QVQLVESGGGVVQPGRSLRLSCAASGFTFSTYGMHWVRQAPGE | 208 |
| VH | GLEWVAVIWDDGSYKYYGDSVKGRFTISRDNSKNTLYLQMNSLRA | |
| | EDTAVYYCARDGITMVRGVMKDYFDYWGQGTLVTVSS | |
| Anti EGFR | AIQLTQSPESLSASVGDRVTITCRASQDISSALVWYQQKPGKAPKL | 209 |
| VL | LIYDASSLESGVPDRFSGSESGTDFTLTISSLQPEDFATYYCQQFN | |
| | SYPLTFGGGTKVEIK | |
| Anti EGFR | GFTFSTYG | 139 |
| HCDR1 | | |
| Anti EGFR | IWDDGSYK | 140 |
| HCDR2 | | |
| Anti EGFR | ARDGITMVRGVMKDYFDY | 141 |
| HCDR3 | | |
| Anti EGFR | QDISSA | 142 |
| LCDR1 | | |
| Anti EGFR | DAS | |
| LCDR2 | | |
| Anti EGFR | QQFNSYPLT | 143 |
| LCDR3 | | |
| Anti cMET | DIVMTQSPDSLAVSLGERATINCKSSESVDSYANSFLHWYQQKPG | 210 |
| hole chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGECGGGGSGG | |
| | GGSGGGGGGGGSGGGGSGGGGSGGGGGGGGSQVQLVQSG | |
| | AEVKKPGASVKVSCKASGYIFTAYTMHWVRQAPGQGLEWMGWIK | |
| | PNNGLANYAQKFQGRVTMTRDTSISTAYMELSRLRSDDTAVYYCA | |
| | RSEITTEFDYWGQGTLVTVSSASTKGPSVFPLAPSSKSTSGGTAAL | |
| | GCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAP | |
| | EAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAKGQPREPQVYTLPPCRDELTKNQVSLSC | |
| | AVKGFYPSDIAVEWESNGQPENNYKTTPPVLDSDGSFFLVSKLTV | |
| | DKSRWQQGNVFSCSVMHEALHNHYTQKSLSLSPGK | |
| Anti cMET | QVQLVQSGAEVKKPGASVKVSCKASGYIFTAYTMHWVRQAPGQG | 211 |
| heavy chain | LEWMGWIKPNNGLANYAQKFQGRVTMTRDTSISTAYMELSRLRS | |
| | DDTAVYYCARSEITTEFDYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDKT | |
| | HTCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDW | |
| | LNGKEYKCKVSNKALPAPIEKTISKAKGQPREPQVYTLPPCRDELT | |
| | KNQVSLSCAVKGFYPSDIAVEWESNGQPENNYKTTPPVLDSDGSF | |
| | FLVSKLTVDKSRWQQGNVFSCSVMHEALHNHYTQKSLSLSPGK | |
| Anti cMET | DIVMTQSPDSLAVSLGERATINCKSSESVDSYANSFLHWYQQKPG | 212 |
| light chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGEC | |
| Anti cMET | QVQLVQSGAEVKKPGASVKVSCKASGYIFTAYTMHWVRQAPGQG | 213 |
| VH | LEWMGWIKPNNGLANYAQKFQGRVTMTRDTSISTAYMELSRLRS | |
| | DDTAVYYCARSEITTEFDYWGQGTLVTVSS | |
| Anti cMET | DIVMTQSPDSLAVSLGERATINCKSSESVDSYANSFLHWYQQKPG | 214 |
| VL | QPPKLLIYRASTRESGVPDRFSGSGSGTDFTLTISSLQAEDVAVYY | |
| | CQQSKEDPLTFGGGTKVEIK | |
| Anti cMET | GYIFTAYT | 149 |
| HCDR1 | | |
| Anti cMET | IKPNNGLA | 150 |
| HCDR2 | | |
| Anti cMET | ARSEITTEFDY | 151 |
| HCDR3 | | |
| Anti cMET | ESVDSYANSF | 152 |
| LCDR1 | | |
| Anti cMET | RAS | |
| LCDR2 | | |
| Anti cMET | QQSKEDPLT | 153 |
| LCDR3 | | |

TABLE 13
Sequences of Bsab 6 WT

| | | SEQ ID |
|---|---|---|
| Description | Amino Acid Sequence | NO: |
| Anti EGFR | AIQLTQSPSSLSASVGDRVTITCRASQDISSALVWYQQKPGKAPKL | 215 |
| knob chain | LIYDASSLESGVPSRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGECGGGGSGGGGSGGG | |
| | GSGGGGSGGGGSGGGGSGGGGSGGGGSQVQLVESGGGVVQP | |
| | GRSLRLSCAASGFTFSTYGMHWVRQAPGKGLEWVAVIWDDGSYK | |
| | YYGDSVKGRFTISRDNSKNTLYLQMNSLRAEDTAVYYCARDGITM | |
| | VRGVMKDYFDYWGQGTLVTVSSASTKGPSVFPLAPSSKSTSGGT | |
| | AALGCLVKDYFPEPVTVSWNSGALTSGVHTFPAVLQSSGLYSLSS | |
| | WTVPSSSLGTQTYICNVNHKPSNTKVDKKVEPKSCDKTHTCPPC | |
| | PAPELLGGPSVFLFPPKPKDTLMISRTPEVTCVVDVSHEDPEVKF | |
| | NWYVDGVEVHNAKTKPREEQYNSTYRVVSVLTVLHQDWLNGKEY | |
| | KCKVSNKALPAPIEKTISKAKGQPREPQVCTLPPSRDELTKNQVSL | |
| | WCLVKGFYPSDIAVEWESNGQPENNYKTTPPVLDSDGSFFLYSKL | |
| | TVDKSRWQQGNVFSCSVMHEALHNHYTQKSLSLSPGK | |
| Anti EGFR | QVQLVESGGGVVQPGRSLRLSCAASGFTFSTYGMHWVRQAPGK | 216 |
| heavy chain | GLEWVAVIWDDGSYKYYGDSVKGRFTISRDNSKNTLYLQMNSLRA | |
| | EDTAVYYCARDGITMVRGVMKDYFDYWGQGTLVTVSSASTKGPS | |
| | VFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKV | |
| | EPKSCDKTHTCPPCPAPELLGGPSVFLFPPKPKDTLMISRTPEVTC | |
| | WWWDVSHEDPEVKFNWYVDGVEVHNAKTKPREEQYNSTYRVVSV | |
| | LTVLHQDWLNGKEYKCKVSNKALPAPIEKTISKAKGQPREPQVCTL | |
| | PPSRDELTKNQVSLWCLVKGFYPSDIAVEWESNGQPENNYKTTPP | |
| | VLDSDGSFFLYSKLTVDKSRWQQGNVFSCSVMHEALHNHYTQKS | |
| | LSLSPGK | |
| Anti EGFR | AIQLTQSPSSLSASVGDRVTITCRASQDISSALVWYQQKPGKAPKL | 217 |
| light chain | LIYDASSLESGVPSRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| Anti EGFR | QVQLVESGGGVVQPGRSLRLSCAASGFTFSTYGMHWVRQAPGK | 218 |
| VH | GLEWVAVIWDDGSYKYYGDSVKGRFTISRDNSKNTLYLQMNSLRA | |
| | EDTAVYYCARDGITMVRGVMKDYFDYWGQGTLVTVSS | |
| Anti EGFR | AIQLTQSPSSLSASVGDRVTITCRASQDISSALVWYQQKPGKAPKL | 219 |
| VL | LIYDASSLESGVPSRFSGSESGTDFTLTISSLQPEDFATYYCQQFN | |
| | SYPLTFGGGTKVEIK | |
| Anti EGFR | GFTFSTYG | 139 |
| HCDR1 | | |
| Anti EGFR | IWDDGSYK | 140 |
| HCDR2 | | |
| Anti EGFR | ARDGITMVRGVMKDYFDY | 141 |
| HCDR3 | | |
| Anti EGFR | QDISSA | 142 |
| LCDR1 | | |
| Anti EGFR | DAS | |
| LCDR2 | | |
| Anti EGFR | QQFNSYPLT | 143 |
| LCDR3 | | |
| Anti cMET | DIVMTQSPDSLAVSLGERATINCKSSESVDSYANSFLHWYQQKPG | 220 |
| hole chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGECGGGGSGG | |
| | GGSGGGGSGGGGSGGGGSGGGGSGGGGSGGGGSQVQLVQSG | |
| | AEVKKPGASVKVSCKASGYIFTAYTMHWVRQAPGQGLEWMGWIK | |
| | PNNGLANYAQKFQGRVTMTRDTSISTAYMELSRLRSDDTAVYYCA | |
| | RSEITTEFDYWGQGTLVTVSSASTKGPSVFPLAPSSKSTSGGTAAL | |
| | GCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAP | |
| | EAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAKGQPREPQVYTLPPCRDELTKNQVSLSC | |
| | AVKGFYPSDIAVEWESNGQPENNYKTTPPVLDSDGSFFLVSKLTV | |
| | DKSRWQQGNVFSCSVMHEALHNHYTQKSLSLSPGK | |
| Anti cMET | QVQLVQSGAEVKKPGASVKVSCKASGYIFTAYTMHWVRQAPGQG | 221 |
| heavy chain | LEWMGWIKPNNGLANYAQKFQGRVTMTRDTSISTAYMELSRLRS | |
| | DDTAVYYCARSEITTEFDYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDKT | |
| | HTCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDW | |
| | LNGKEYKCKVSNKALPAPIEKTISKAKGQPREPQVYTLPPCRDELT | |
| | KNQVSLSCAVKGFYPSDIAVEWESNGQPENNYKTTPPVLDSDGSF | |
| | FLVSKLTVDKSRWQQGNVFSCSVMHEALHNHYTQKSLSLSPGK | |
| Anti cMET | DIVMTQSPDSLAVSLGERATINCKSSESVDSYANSFLHWYQQKPG | 222 |
| light chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGEC | |
| Anti cMET | QVQLVQSGAEVKKPGASVKVSCKASGYIFTAYTMHWVRQAPGQG | 223 |
| VH | LEWMGWIKPNNGLANYAQKFQGRVTMTRDTSISTAYMELSRLRS | |
| | DDTAVYYCARSEITTEFDYWGQGTLVTVSS | |
| Anti cMET | DIVMTQSPDSLAVSLGERATINCKSSESVDSYANSFLHWYQQKPG | 224 |
| VL | QPPKLLIYRASTRESGVPDRFSGSGSGTDFTLTISSLQAEDVAVYY | |
| | CQQSKEDPLTFGGGTKVEIK | |
| Anti cMET | GYIFTAYT | 149 |
| HCDR1 | | |
| Anti cMET | IKPNNGLA | 150 |
| HCDR2 | | |
| Anti cMET | ARSEITTEFDY | 151 |
| HCDR3 | | |
| Anti cMET | ESVDSYANSF | 152 |
| LCDR1 | | |
| Anti cMET | RAS | |
| LCDR2 | | |
| Anti cMET | QQSKEDPLT | 153 |
| LCDR3 | | |

IAA

TABLE 14
Sequences of Bsab 52 WT

| | | SEQ ID |
|---|---|---|
| Description | Amino Acid Sequence | NO: |
| Anti EGFR | AIQLTQSPSSLSASVGDRVTITCRASQDISSALVWYQQKPGKAPKL | 225 |
| knob chain | LIYDASSLESGVPSRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGECGGGGSGGGGSGGG | |
| | GSGGGGSGGGGSGGGGGGGGSGGGGSQVQLVESGGGVVQP | |
| | GRSLRLSCAASGFTFSTYGMHWVRQAPGKGLEWVAVIWDDGSYK | |
| | YYGDSVKGRFTISRDNSKNTLYLQMNSLRAEDTAVYYCARDGITM | |
| | VRGVMKDYFDYWGQGTLVTVSSASTKGPSVFPLAPSSKSTSGGT | |
| | AALGCLVKDYFPEPVTVSWNSGALTSGVHTFPAVLQSSGLYSLSS | |
| | VTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEY | |
| | KCKVSNKALPAPIEKTISKAKGQPREPQVCTLPPSRDELTKNQVSL | |
| | WCLVKGFYPSDIAVEWESNGQPENNYKTTPPVLDSDGSFFLYSKL | |
| | TVDKSRWQQGNVFSCSVMHEALHNHYTQKSLSLSPGK | |
| Anti EGFR | QVQLVESGGGVVQPGRSLRLSCAASGFTFSTYGMHWVRQAPGK | 226 |
| heavy chain | GLEWVAVIWDDGSYKYYGDSVKGRFTISRDNSKNTLYLQMNSLRA | |
| | EDTAVYYCARDGITMVRGVMKDYFDYWGQGTLVTVSSASTKGPS | |
| | VFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKV | |
| | EPKSCDKTHTCPPCPAPELLGGPSVFLFPPKPKDTLMISRTPEVTC | |
| | WVDVSHEDPEVKFNWYVDGVEVHNAKTKPREEQYNSTYRVVSV | |
| | LTVLHQDWLNGKEYKCKVSNKALPAPIEKTISKAKGQPREPQVCTL | |
| | PPSRDELTKNQVSLWCLVKGFYPSDIAVEWESNGQPENNYKTTPP | |
| | VLDSDGSFFLYSKLTVDKSRWQQGNVFSCSVMHEALHNHYTQKS | |
| | LSLSPGK | |
| Anti EGFR | AIQLTQSPSSLSASVGDRVTITCRASQDISSALVWYQQKPGKAPKL | 227 |
| light chain | LIYDASSLESGVPSRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| Anti EGFR | QVQLVESGGGVVQPGRSLRLSCAASGFTFSTYGMHWVRQAPGK | 228 |
| VH | GLEWVAVIWDDGSYKYYGDSVKGRFTISRDNSKNTLYLQMNSLRA | |
| | EDTAVYYCARDGITMVRGVMKDYFDYWGQGTLVTVSS | |
| Anti EGFR | AIQLTQSPSSLSASVGDRVTITCRASQDISSALVWYQQKPGKAPKL | 229 |
| VL | LIYDASSLESGVPSRFSGSESGTDFTLTISSLQPEDFATYYCQQFN | |
| | SYPLTFGGGTKVEIK | |
| Anti EGFR | GFTFSTYG | 139 |
| HCDR1 | | |
| Anti EGFR | IWDDGSYK | 140 |
| HCDR2 | | |
| Anti EGFR | ARDGITMVRGVMKDYFDY | 141 |
| HCDR3 | | |
| Anti EGFR | QDISSA | 142 |
| LCDR1 | | |
| Anti EGFR | DAS | |
| LCDR2 | | |
| Anti EGFR | QQFNSYPLT | 143 |
| LCDR3 | | |
| Anti cMET | DIQMTQSPSSVSASVGDRVTITCRASQGISNWLAWFQHKPGKAPK | 230 |
| hole chain | LLIYAASSLLSGVPSRFSGSGSGTDFTLTISSLQPEDFATYYCQQ | |
| | ANSFPITFGQGTRLEIKRTVAAPSVFIFPPSDEQLKSGTASVVCL | |
| | LNNFYPREAKVQWKVDNALQSGNSQESVTEQDSKDSTYSLSSTLT | |
| | LSKADYEKHKVYACEVTHQGLSSPVTKSFNRGECGGGGSGGGGSG | |
| | GGGSGGGGSGGGGSGGGGSGGGGSGGGGSQVQLVQSGAEVKKP | |
| | GASVKVSCETSGYTFTSYGISWVRQAPGHGLEWMGWISAYNGYT | |
| | NYAQKLQGRVTMTTDTSTSTAYMELRSLRSDDTAVYYCARDLRGT | |
| | NYFDYWGQGTLVTVSSASTKGPSVFPLAPSSKSTSGGTAALGCLV | |
| | KDYFPEPVTVSWNSGALTSGVHTFPAVLQSSGLYSLSSVVTVPSS | |
| | SLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNK | |
| | ALPAPIEKTISKAKGQPREPQVYTLPPCRDELTKNQVSLSCAVKG | |
| | FYPSDIAVEWESNGQPENNYKTTPPVLDSDGSFFLVSKLTVDKSR | |
| | WQQGNVFSCSVMHEALHNHYTQKSLSLSPGK | |
| Anti cMET | QVQLVQSGAEVKKPGASVKVSCETSGYTFTSYGISWVRQAPGHG | 231 |
| heavy chain | LEWMGWISAYNGYTNYAQKLQGRVTMTTDTSTSTAYMELRSLRS | |
| | DDTAVYYCARDLRGTNYFDYWGQGTLVTVSSASTKGPSVFPLAPS | |
| | SKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCDK | |
| | THTCPPCPAPELLGGPSVFLFPPKPKDTLMISRTPEVTCVVVDVSH | |
| | EDPEVKFNWYVDGVEVHNAKTKPREEQYNSTYRVVSVLTVLHQD | |
| | WLNGKEYKCKVSNKALPAPIEKTISKAKGQPREPQVYTLPPCRDEL | |
| | TKNQVSLSCAVKGFYPSDIAVEWESNGQPENNYKTTPPVLDSDGS | |
| | FFLVSKLTVDKSRWQQGNVFSCSVMHEALHNHYTQKSLSLSPGK | |
| Anti cMET | DIQMTQSPSSVSASVGDRVTITCRASQGISNWLAWFQHKPGKAPKL | 232 |
| light chain | LIYAASSLLSGVPSRFSGSGSGTDFTLTISSLQPEDFATYYCQQAN | |
| | SFPITFGQGTRLEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| Anti cMET | QVQLVQSGAEVKKPGASVKVSCETSGYTFTSYGISWVRQAPGHG | 233 |
| VH | LEWMGWISAYNGYTNYAQKLQGRVTMTTDTSTSTAYMELRSLRS | |
| | DDTAVYYCARDLRGTNYFDYWGQGTLVTVSS | |
| Anti cMET | DIQMTQSPSSVSASVGDRVTITCRASQGISNWLAWFQHKPGKAPK | 234 |
| VL | LLIYAASSLLSGVPSRFSGSGSGTDFTLTISSLQPEDFATYYCQQ | |
| | ANSFPITFGQGTRLEIK | |
| Anti cMET | GYTFTSYG | 235 |
| HCDR1 | | |
| Anti cMET | ISAYNGYT | 236 |
| HCDR2 | | |
| Anti cMET | ARDLRGTNYFDY | 237 |
| HCDR3 | | |
| Anti cMET | QGISNWL | 238 |
| LCDR1 | | |
| Anti cMET | AAS | |
| LCDR2 | | |
| Anti cMET | QQANSFPIT | 239 |
| LCDR3 | | |

TABLE 15
Sequences of Bsab 67 WT

| | | SEQ ID |
|---|---|---|
| Description | Amino Acid Sequence | NO: |
| Anti EGFR | AIQLTQSPESLSASVGDRVTITCRASQDISSALVWYQQKPGKAPKL | 240 |
| knob chain | LIYDASSLESGVPDRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGECGGGGSGGGGSGGG | |
| | GSGGGGSGGGGSGGGGSGGGGSGGGGSQVQLVESGGGVVQP | |
| | GRSLRLSCAASGFTFSTYGMHWVRQAPGQGLEWVAVIWDDGSY | |
| | KYYGDSVKGRFTISRDNSKNTLYLQMNSLRAEDTAVYYCARDGIT | |
| | MVRGVMKDYFDYWGQGTLVTVSSASTKGPSVFPLAPSSKSTSGG | |
| | TAALGCLVKDYFPEPVTVSWNSGALTSGVHTFPAVLQSSGLYSLS | |
| | SWVTVPSSSLGTQTYICNVNHKPSNTKVDKKVEPKSCDKTHTCPP | |
| | CPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKE | |
| | YKCKVSNKALPAPIEKTISKAKGQPREPQVCTLPPSRDELTKNQVS | |
| | LWCLVKGFYPSDIAVEWESNGQPENNYKTTPPVLDSDGSFFLYSK | |
| | LTVDKSRWQQGNVFSCSVMHEALHNHYTQKSLSLSPGK | |
| Anti EGFR | QVQLVESGGGVVQPGRSLRLSCAASGFTFSTYGMHWVRQAPGQ | 241 |
| heavy chain | GLEWVAVIWDDGSYKYYGDSVKGRFTISRDNSKNTLYLQMNSLRA | |
| | EDTAVYYCARDGITMVRGVMKDYFDYWGQGTLVTVSSASTKGPS | |
| | VFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKV | |
| | EPKSCDKTHTCPPCPAPELLGGPSVFLFPPKPKDTLMISRTPEVTC | |
| | WVDVSHEDPEVKFNWYVDGVEVHNAKTKPREEQYNSTYRVVSV | |
| | LTVLHQDWLNGKEYKCKVSNKALPAPIEKTISKAKGQPREPQVCTL | |
| | PPSRDELTKNQVSLWCLVKGFYPSDIAVEWESNGQPENNYKTTPP | |
| | VLDSDGSFFLYSKLTVDKSRWQQGNVFSCSVMHEALHNHYTQKS | |
| | LSLSPGK | |
| Anti EGFR | AIQLTQSPESLSASVGDRVTITCRASQDISSALVWYQQKPGKAPKL | 242 |
| light chain | LIYDASSLESGVPDRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| Anti EGFR | QVQLVESGGGVVQPGRSLRLSCAASGFTFSTYGMHWVRQAPGQ | 243 |
| VH | GLEWVAVIWDDGSYKYYGDSVKGRFTISRDNSKNTLYLQMNSLRA | |
| | EDTAVYYCARDGITMVRGVMKDYFDYWGQGTLVTVSS | |
| Anti EGFR | AIQLTQSPESLSASVGDRVTITCRASQDISSALVWYQQKPGKAPKL | 244 |
| VL | LIYDASSLESGVPDRFSGSESGTDFTLTISSLQPEDFATYYCQQFN | |
| | SYPLTFGGGTKVEIK | |
| Anti EGFR | GFTFSTYG | 139 |
| HCDR1 | | |
| Anti EGFR | IWDDGSYK | 140 |
| HCDR2 | | |
| Anti EGFR | ARDGITMVRGVMKDYFDY | 141 |
| HCDR3 | | |
| Anti EGFR | QDISSA | 142 |
| LCDR1 | | |
| Anti EGFR | DAS | |
| LCDR2 | | |
| Anti EGFR | QQFNSYPLT | 143 |
| LCDR3 | | |
| Anti cMET | DIVMTQSPDSLAVSLGERATINCKSSESVDSYANSFLHWYQQKPG | 245 |
| hole chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGECGGGGSGG | |
| | GGSGGGGGGGGSGGGGSGGGGSGGGGSGGGGSQVQLVQSG | |
| | AEVKKPGASVKVSCKASGYIFTAYTMHWVRQAPGQGLEWMGWIK | |
| | PNNGLANYAQKFQGRVTMTRDTSISTAYMELSRLRSDDTAVYYCA | |
| | RSEITTEFDYWGQGTLVTVSSASTKGPSVFPLAPSSKSTSGGTAAL | |
| | GCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCK | |
| | VSNKALPAPIEKTISKAKGQPREPQVYTLPPCRDELTKNQVSLSCA | |
| | VKGFYPSDIAVEWESNGQPENNYKTTPPVLDSDGSFFLVSKLTVD | |
| | KSRWQQGNVFSCSVMHEALHNHYTQKSLSLSPGK | |
| Anti cMET | QVQLVQSGAEVKKPGASVKVSCKASGYIFTAYTMHWVRQAPGQG | 246 |
| heavy chain | LEWMGWIKPNNGLANYAQKFQGRVTMTRDTSISTAYMELSRLRS | |
| | DDTAVYYCARSEITTEFDYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDW | |
| | LNGKEYKCKVSNKALPAPIEKTISKAKGQPREPQVYTLPPCRDELT | |
| | KNQVSLSCAVKGFYPSDIAVEWESNGQPENNYKTTPPVLDSDGSF | |
| | FLVSKLTVDKSRWQQGNVFSCSVMHEALHNHYTQKSLSLSPGK | |
| Anti cMET | DIVMTQSPDSLAVSLGERATINCKSSESVDSYANSFLHWYQQKPG | 247 |
| light chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGEC | |
| Anti cMET | QVQLVQSGAEVKKPGASVKVSCKASGYIFTAYTMHWVRQAPGQG | 248 |
| VH | LEWMGWIKPNNGLANYAQKFQGRVTMTRDTSISTAYMELSRLRS | |
| | DDTAVYYCARSEITTEFDYWGQGTLVTVSS | |
| Anti cMET | DIVMTQSPDSLAVSLGERATINCKSSESVDSYANSFLHWYQQKPG | 249 |
| VL | QPPKLLIYRASTRESGVPDRFSGSGSGTDFTLTISSLQAEDVAVYY | |
| | CQQSKEDPLTFGGGTKVEIK | |
| Anti cMET | GYIFTAYT | 149 |
| HCDR1 | | |
| Anti cMET | IKPNNGLA | 150 |
| HCDR2 | | |
| Anti cMET | ARSEITTEFDY | 151 |
| HCDR3 | | |
| Anti cMET | ESVDSYANSF | 152 |
| LCDR1 | | |
| Anti cMET | RAS | |
| LCDR2 | | |
| Anti cMET | QQSKEDPLT | 153 |
| LCDR3 | | |

TABLE 16
Sequences of Bsab 68WT

| | | SEQ ID |
|---|---|---|
| Description | Amino Acid Sequence | NO: |
| Anti EGFR | QVQLVESGGGVVQPGRSLRLSCAASGFTFSTYGMHWVREAPGK | 250 |
| knob chain | DLEWVAVIWDDGSYKYYGDSVKGRFTISRDNSKNTLYLQMNSLRA | |
| | EDTAVYYCARDGITMVRGVMKDYFDYWGQGTLVTVSSGGGGSG | |
| | GGGSGGGGSGGGGSAIQLTQSPSSLSASVGDRVTITCRASQDISS | |
| | ALVWYQKKPGKAPKLLIYDASSLESGVPSRFSGSESGTDFTLTISS | |
| | LQPEDFATYYCQQFNSYPLTFGKGTKVEIKGGGGSEPKSCDKTHT | |
| | CPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLN | |
| | GKEYKCKVSNKALPAPIEKTISKAKGQPREPQVCTLPPSRDELTKN | |
| | QVSLWCLVKGFYPSDIAVEWESNGQPENNYKTTPPVLDSDGSFFL | |
| | YSKLTVDKSRWQQGNVFSCSVMHEALHNHYTQKSLSLSPGK | |
| Anti EGFR | QVQLVESGGGVVQPGRSLRLSCAASGFTFSTYGMHWVREAPGK | 251 |
| VH | DLEWVAVIWDDGSYKYYGDSVKGRFTISRDNSKNTLYLQMNSLRA | |
| | EDTAVYYCARDGITMVRGVMKDYFDYWGQGTLVTVSS | |
| Anti EGFR | AIQLTQSPSSLSASVGDRVTITCRASQDISSALVWYQKKPGKAPKL | 252 |
| VL | LIYDASSLESGVPSRFSGSESGTDFTLTISSLQPEDFATYYCQQFN | |
| | SYPLTFGKGTKVEIK | |
| Anti EGFR | GFTFSTYG | 139 |
| HCDR1 | | |
| Anti EGFR | IWDDGSYK | 140 |
| HCDR2 | | |
| Anti EGFR | ARDGITMVRGVMKDYFDY | 141 |
| HCDR3 | | |
| Anti EGFR | QDISSA | 142 |
| LCDR1 | | |
| Anti EGFR | DAS | |
| LCDR2 | | |
| Anti EGFR | QQFNSYPLT | 143 |
| LCDR3 | | |
| Anti cMET | QVQLVQSGAEVKKPGASVKVSCKASGYIFTAYTMHWVRQAPGQK | 253 |
| hole chain | LEWMGWIKPNNGLANYAQKFQGRVTMTRDTSISTAYMELSRLRS | |
| | DDTAVYYCARSEITTEFDYWGQGTLVTVSSGGGGSGGGGSGGG | |
| | GSGGGGSDIVMTQSPDSLAVSLGERATINCKSSESVDSYANSFLH | |
| | WYQQKPGQPPKLLIYRASTRESGVPDRFSGSGSGTDFTLTISSLQ | |
| | AEDVAVYYCQQSKEDPLTFGDGTKVEIKGGGGSEPKSCDKTHTCP | |
| | PCPAPELLGGPSVFLFPPKPKDTLMISRTPEVTCVWVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVYTLPPCRDELTKNQV | |
| | SLSCAVKGFYPSDIAVEWESNGQPENNYKTTPPVLDSDGSFFLVS | |
| | KLTVDKSRWQQGNVFSCSVMHEALHNHYTQKSLSLSPGK | |
| Anti cMET | QVQLVQSGAEVKKPGASVKVSCKASGYIFTAYTMHWVRQAPGQK | 254 |
| VH | LEWMGWIKPNNGLANYAQKFQGRVTMTRDTSISTAYMELSRLRS | |
| | DDTAVYYCARSEITTEFDYWGQGTLVTVSS | |
| Anti cMET | DIVMTQSPDSLAVSLGERATINCKSSESVDSYANSFLHWYQQKPG | 255 |
| VL | QPPKLLIYRASTRESGVPDRFSGSGSGTDFTLTISSLQAEDVAVYY | |
| | CQQSKEDPLTFGDGTKVEIK | |
| Anti cMET | GYIFTAYT | 149 |
| HCDR1 | | |
| Anti cMET | IKPNNGLA | 150 |
| HCDR2 | | |
| Anti cMET | ARSEITTEFDY | 151 |
| HCDR3 | | |
| Anti cMET | ESVDSYANSF | 152 |
| LCDR1 | | |
| Anti cMET | RAS | |
| LCDR2 | | |
| Anti cMET | QQSKEDPLT | 153 |
| LCDR3 | | |

TABLE 17
Sequences of Bsab 81 WT

| | | SEQ ID |
|---|---|---|
| Description | Amino Acid Sequence | NO: |
| Anti EGFR | AIQLTQSPESLSASVGDRVTITCRASQDISSALVWYQQKPGKAPKL | 256 |
| knob chain | LIYDASSLESGVPDRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGECGGGGSGGGGSGGG | |
| | GSGGGGSGGGGSGGGGSGGGGSGGGGSQVQLVESGGGVVQP | |
| | GRSLRLSCAASGFTFSTYGMHWVRQAPGEGLEWVAVIWDDGSYK | |
| | YYGDSVKGRFTISRDNSKNTLYLQMNSLRAEDTAVYYCARDGITM | |
| | VRGVMKDYFDYWGQGTLVTVSSASTKGPSVFPLAPSSKSTSGGT | |
| | AALGCLVKDYFPEPVTVSWNSGALTSGVHTFPAVLQSSGLYSLSS | |
| | WTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEY | |
| | KCKVSNKALPAPIEKTISKAKGQPREPQVCTLPPSRDELTKNQVSL | |
| | WCLVKGFYPSDIAVEWESNGQPENNYKTTPPVLDSDGSFFLYSKL | |
| | TVDKSRWQQGNVFSCSVMHEALHNHYTQKSLSLSPGK | |
| Anti EGFR | QVQLVESGGGVVQPGRSLRLSCAASGFTFSTYGMHWVRQAPGE | 257 |
| heavy chain | GLEWVAVIWDDGSYKYYGDSVKGRFTISRDNSKNTLYLQMNSLRA | |
| | EDTAVYYCARDGITMVRGVMKDYFDYWGQGTLVTVSSASTKGPS | |
| | VFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKV | |
| | EPKSCDKTHTCPPCPAPELLGGPSVFLFPPKPKDTLMISRTPEVTC | |
| | WVDVSHEDPEVKFNWYVDGVEVHNAKTKPREEQYNSTYRVVSV | |
| | LTVLHQDWLNGKEYKCKVSNKALPAPIEKTISKAKGQPREPQVCTL | |
| | PPSRDELTKNQVSLWCLVKGFYPSDIAVEWESNGQPENNYKTTPP | |
| | VLDSDGSFFLYSKLTVDKSRWQQGNVFSCSVMHEALHNHYTQKS | |
| | LSLSPGK | |
| Anti EGFR | AIQLTQSPESLSASVGDRVTITCRASQDISSALVWYQQKPGKAPKL | 258 |
| light chain | LIYDASSLESGVPDRFSGSESGTDFTLTISSLQPEDFATYYCQQFN | |
| | SYPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| Anti EGFR | QVQLVESGGGVVQPGRSLRLSCAASGFTFSTYGMHWVRQAPGE | 259 |
| VH | GLEWVAVIWDDGSYKYYGDSVKGRFTISRDNSKNTLYLQMNSLRA | |
| | EDTAVYYCARDGITMVRGVMKDYFDYWGQGTLVTVSS | |
| Anti EGFR | AIQLTQSPESLSASVGDRVTITCRASQDISSALVWYQQKPGKAPKL | 260 |
| VL | LIYDASSLESGVPDRFSGSESGTDFTLTISSLQPEDFATYYCQQFN | |
| | SYPLTFGGGTKVEIK | |
| Anti EGFR | GFTFSTYG | 139 |
| HCDR1 | | |
| Anti EGFR | IWDDGSYK | 140 |
| HCDR2 | | |
| Anti EGFR | ARDGITMVRGVMKDYFDY | 141 |
| HCDR3 | | |
| Anti EGFR | QDISSA | 142 |
| LCDR1 | | |
| Anti EGFR | DAS | |
| LCDR2 | | |
| Anti EGFR | QQFNSYPLT | 143 |
| LCDR3 | | |
| Anti cMET | DIVMTQSPDSLAVSLGERATINCKSSESVDSYANSFLHWYQQKPG | 261 |
| hole chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGECGGGGSGG | |
| | GGSGGGGSGGGGSGGGGSGGGGSGGGGSGGGGSQVQLVQSG | |
| | AEVKKPGASVKVSCKASGYIFTAYTMHWVRQAPGQGLEWMGWIK | |
| | PNNGLANYAQKFQGRVTMTRDTSISTAYMELSRLRSDDTAVYYCA | |
| | RSEITTEFDYWGQGTLVTVSSASTKGPSVFPLAPSSKSTSGGTAAL | |
| | GCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAP | |
| | EAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAKGQPREPQVYTLPPCRDELTKNQVSLSC | |
| | AVKGFYPSDIAVEWESNGQPENNYKTTPPVLDSDGSFFLVSKLTV | |
| | DKSRWQQGNVFSCSVMHEALHNHYTQKSLSLSPGK | |
| Anti cMET | QVQLVQSGAEVKKPGASVKVSCKASGYIFTAYTMHWVRQAPGQG | 262 |
| heavy chain | LEWMGWIKPNNGLANYAQKFQGRVTMTRDTSISTAYMELSRLRS | |
| | DDTAVYYCARSEITTEFDYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDKT | |
| | HTCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDW | |
| | LNGKEYKCKVSNKALPAPIEKTISKAKGQPREPQVYTLPPCRDELT | |
| | KNQVSLSCAVKGFYPSDIAVEWESNGQPENNYKTTPPVLDSDGSF | |
| | FLVSKLTVDKSRWQQGNVFSCSVMHEALHNHYTQKSLSLSPGK | |
| Anti cMET | DIVMTQSPDSLAVSLGERATINCKSSESVDSYANSFLHWYQQKPG | 263 |
| light chain | QPPKLLIYRASTRESGVPDRFSGSGSGTDFTLTISSLQAEDVAVYY | |
| | CQQSKEDPLTFGGGTKVEIKRTVAAPSVFIFPPSDEQLKSGTASVV | |
| | CLLNNFYPREAKVQWKVDNALQSGNSQESVTEQDSKDSTYSLSS | |
| | TLTLSKADYEKHKVYACEVTHQGLSSPVTKSFNRGEC | |
| Anti cMET | QVQLVQSGAEVKKPGASVKVSCKASGYIFTAYTMHWVRQAPGQG | 264 |
| VH | LEWMGWIKPNNGLANYAQKFQGRVTMTRDTSISTAYMELSRLRS | |
| | DDTAVYYCARSEITTEFDYWGQGTLVTVSS | |
| Anti cMET | DIVMTQSPDSLAVSLGERATINCKSSESVDSYANSFLHWYQQKPG | 265 |
| VL | QPPKLLIYRASTRESGVPDRFSGSGSGTDFTLTISSLQAEDVAVYY | |
| | CQQSKEDPLTFGGGTKVEIK | |
| Anti cMET | GYIFTAYT | 149 |
| HCDR1 | | |
| Anti cMET | IKPNNGLA | 150 |
| HCDR2 | | |
| Anti cMET | ARSEITTEFDY | 151 |
| HCDR3 | | |
| Anti cMET | ESVDSYANSF | 152 |
| LCDR1 | | |
| Anti cMET | RAS | |
| LCDR2 | | |
| Anti cMET | QQSKEDPLT | 153 |
| LCDR3 | | |

### Example 11: Binding Activity of Bsab 67

The binding activity of BsAb 67 was tested for both targets. cMET antigen at 2 μg/mL in assay buffer (PBS with 0.05% Tween 20) was initially captured on NI-NTA biosensors. Following a washing step to remove any unbound protein, the respective loaded biosensors were subjected to successive association and dissociation interactions, first with 33.3 nM of the antibodies and then with the EGFR-Fc antigen at 422 nM. Association and dissociation curves were calculated from a non-linear fit of the data using the Acquisition 6.3 software (ForteBio).

As shown in FIG. 3, Bsab 67 shows concurrent binding for both targets.

### Example 12: HGF and EGF Blocking Test

Microtiter 96-well Maxisorp plates (Nunc) were coated with 2 μg/mL human EGFR-Fc or human cMET-Fc in coating buffer (PBS, pH7.4) overnight at 4° C. After blocking (2% BSA in 0.05% TBST), plates were washed three times with wash buffer (0.05% TBST). 100 μL serial diluted test articles were added and incubated for 1 h at room temperature. Plates were washed for 3 times, human HGF-His or EGF-His protein was added and incubated for 1 h at room temperature. After wash, 100 μL HRP conjugated anti-His antibody solution (SinoBiological, 105327-MM02T-H, diluted with blocking buffer) was added to each well. The plates were incubated at room temperature for 1 h and then washed for 3 times. 100 μL TMB solution was added to each well, placed at room temperature for 5-15 min, then the stop solution (2M H₂SO₄) was added with 50 μL per well. Finally, absorbance was measured by microplate reader at 450 nm and 630 nm.

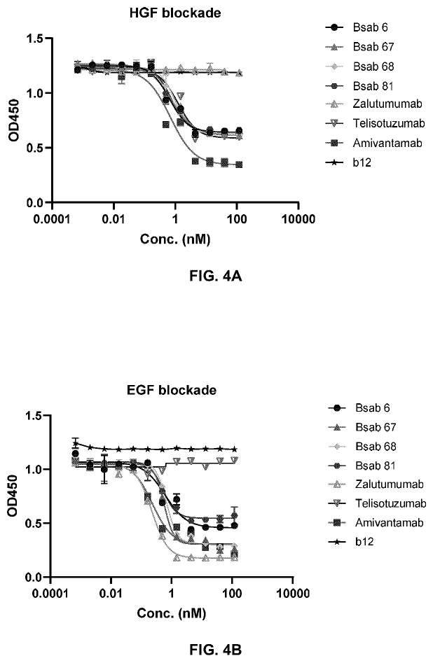

As shown in FIGS. 4A-4B, all optimized bispecific antibodies have good HGF and EGF blocking test.

### Example 13: Binding Activity of Bsab 6 and 12

EGFR and cMET co-expressed tumor cell lines including EBC-1 (JCRB JCRB0820, Provided by Shanghai EK-Bioscience), NCI-H1975 (ATCC® CRL-5908, Provided by Procell), MKN-45 (CL-0292, Provided by Procell), NCI-H1993 (ATCC® CRL-5909, Provided by Shanghai EK-Bioscience), A549 (ATCC® CCL-185, Provided by Procell) were selected for evaluating antibody binding activity by flow cytometry. EBC-1 was cultured with DMEM medium (Gibco, Cat #C11995500BT) containing 10% FBS. NCI-H1975, NCI-H1993 and MKN-45 were cultured with RPMI 1640 (Gibco, Cat #11875093) containing 10% FBS. A549 was cultured with F12K medium (Gibco, Cat #21127030) containing 10% FBS. Each antibody was incubated with different cell lines (1×10⁵ cells/well) for 30 min in 0.2 mL FACS buffer (1×PBS with 0.1% BSA) at 4° C. After wash, cells were incubated with 100 μL PE-conjugated anti human Fc (Abcam, Ab98596, 1:500 dilution) for 30 min at 4° C. After wash with PBS, cells were resuspended in FACS buffer and analyzed by flow cytometer (Beckman, CytoFLEX).

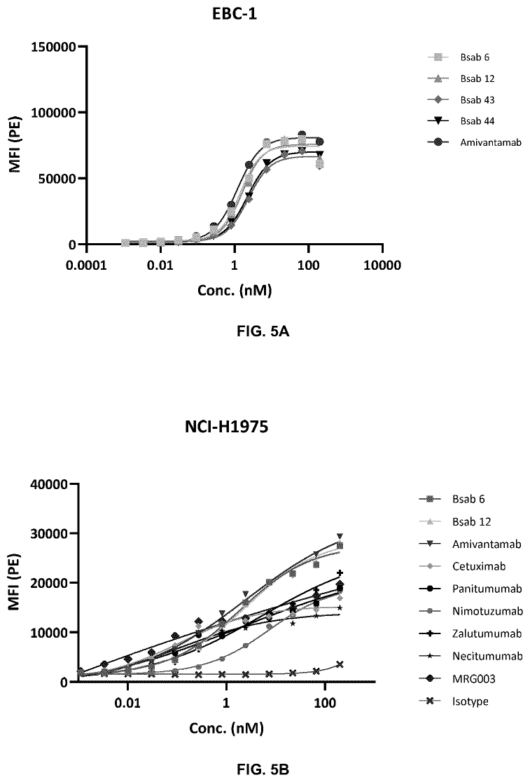

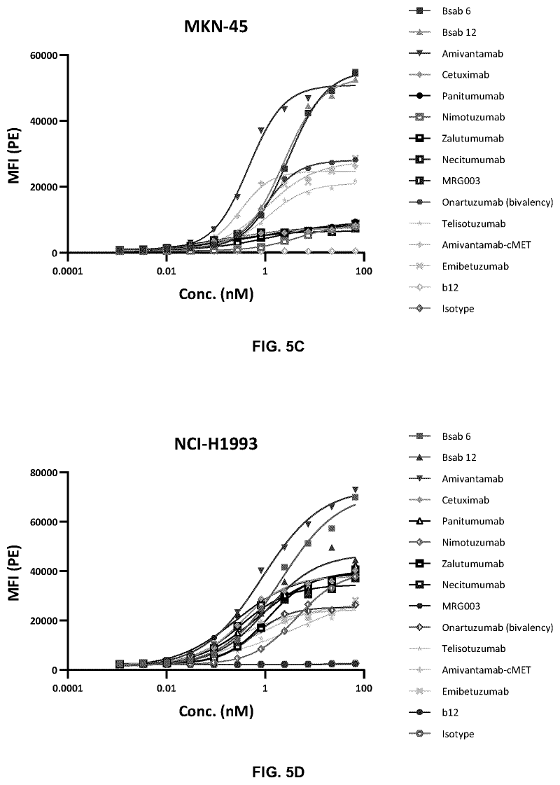

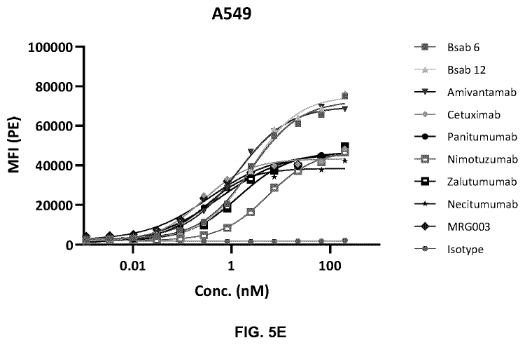

The binding activity of Bsab 6 and Bsab 12 in various tumor cell lines, compared to other bispecific antibodies as shown in FIGS. 5A-5E, Bsab 6 and 12 showed good binding activity, better than monospecific mAb.

### Example 14: Binding Activity of Bsab 6, 52, 67, 68, and 81

EGFR and cMET co-expressed tumor cell lines including EBC-1 (JCRB JCRB0820, Provided by Shanghai EK-Bioscience), NCI-H1975 (ATCC® CRL-5908, Provided by Procell), HCC-827 (ATCC® CRL-2868, Provided by Procell), MKN-45 (CL-0292, Provided by Procell), NCI-N87 (ATCC® CRL-5822, Provided by CRO) were selected for evaluating antibody binding activity by flow cytometry. EBC-1 was cultured with DMEM medium (Gibco, Cat #C11995500BT) containing 10% FBS. NCI-H1975, NCI-N87 and MKN-45 were cultured with RPMI 1640 (Gibco, Cat #11875093) containing 10% FBS. A549 was cultured with F12K medium (Gibco, Cat #21127030) containing 10% FBS. Each antibody was incubated with different cell lines (1×10⁵ cells/well) for 30 mins in 0.2 mL FACS buffer (1×PBS with 0.1% BSA) at 4° C. After wash, cells were incubated with 100 μL PE-conjugated anti human Fc (Abcam, Ab98596, 1:500 dilution) for 30 mins at 4° C. After wash with PBS, cells were resuspended in FACS buffer and analyzed by flow cytometer (Beckman, CytoFLEX).

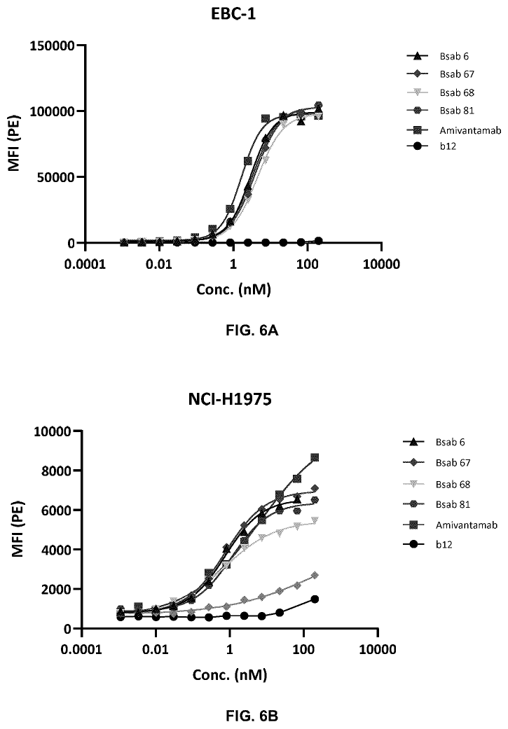

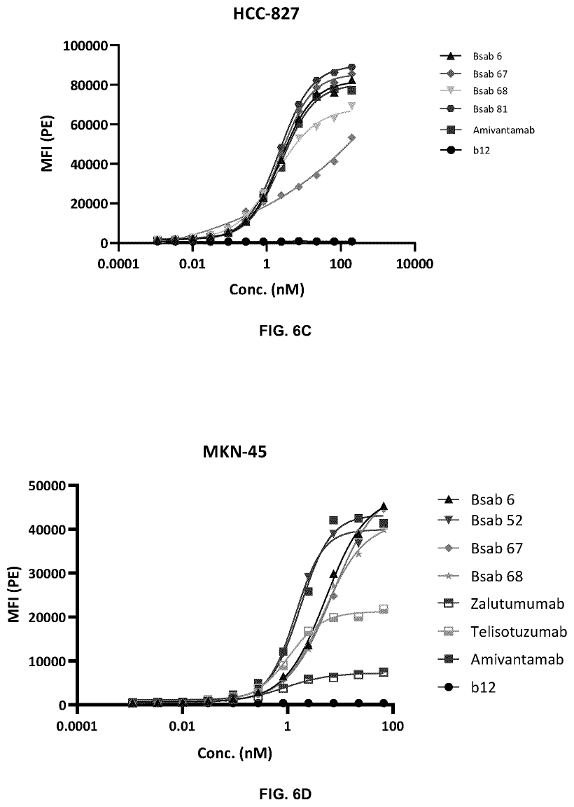

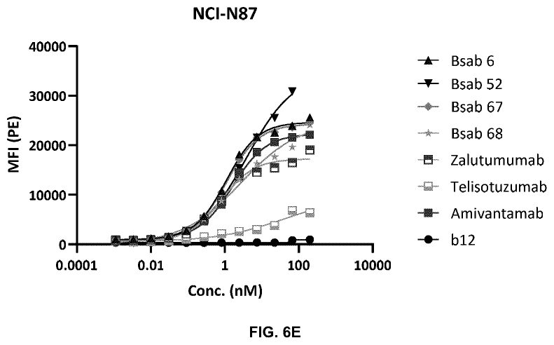

As shown in FIGS. 6A-6E, Bsab 6, 67, 68 and 81 showed similar binding activity in multiple cell lines; Bsab 52 showed comparable binding activity to Amivantamab.

### Example 15: Internalization of Bispecific Antibodies

EGFR cells were harvested and seeded into 96-well plates (Nunc, cat 167008) for overnight culture with the density of 10000 cells/well in 50 μL culture medium. The test articles were mixed with Zenon™ pHrodo™ iFL IgG Labeling Reagent (Invitrogen™, Cat #Z25611) at molar ratio 1:6 and incubated for 30 min at 37° C. to form labeling complexes. 50 μL of labeling complexes (6.67 nM test articles) were incubated with cells at 37° C. At progressive time points 0 h, 2.5 h, 18 h, 24 h and 48 h, cells were analyzed by flow cytometry. Internalization was evaluated by the mean fluorescence intensity (MFI).

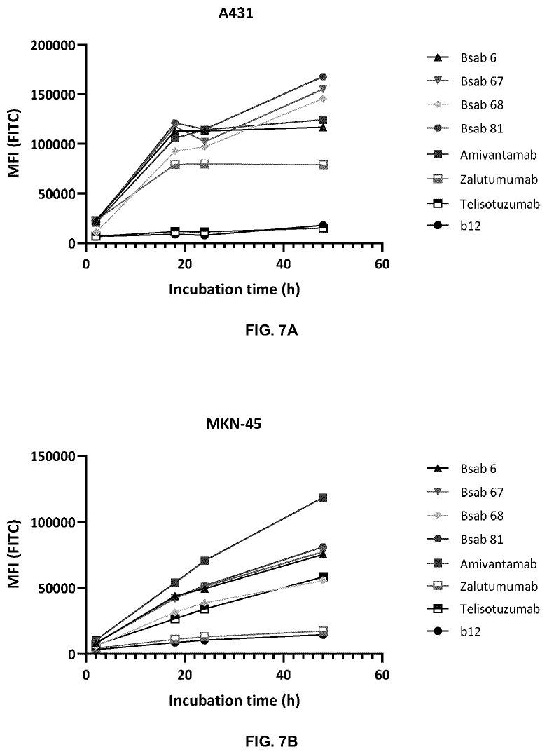

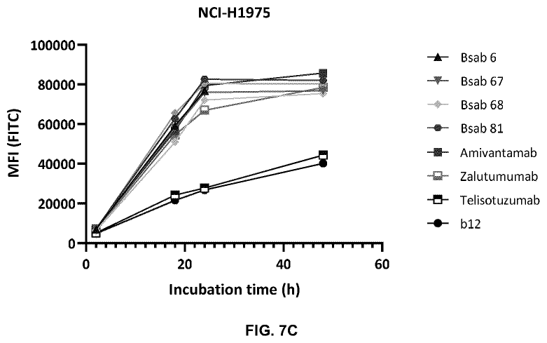

As shown in FIGS. 7A-7C, all bispecifics showed more internalization using pH rodo method than parent mAbs.

### Example 16: In Vitro Cytotoxicity of Bispecific Antibodies

Cells were harvested and seeded into 96-well plates (Coring, 3917) for overnight culture. In the next day, serial diluted testing articles (Bsab 6-LD343 (8), Bsab 67-LD343 (8), Bsab 68-LD343 (4), Bsab 81-LD343 (8), Zalutumumab-LD343 (8), Telisotuzumab-LD343 (8) and b12-LD343 (8)) were added to cells and the incubation was lasting for 96 h at 37° C. After that, 40 μL CellTiter-Glo (Promega) was added to each well and incubated for 5 min at room temperature. Luciferase readings were collected by microplate reader. All readings were normalized as percentage of viable cells in the untreated control wells and the IC₅₀ values were calculated by GraphPad Prism software.

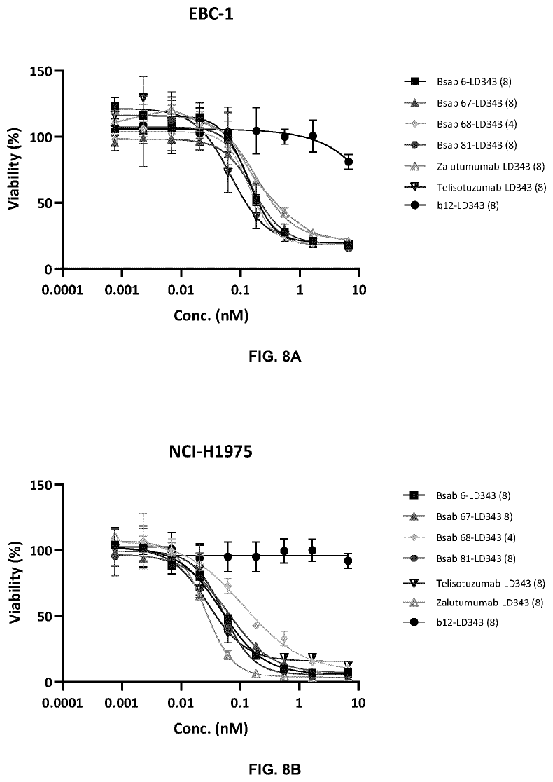

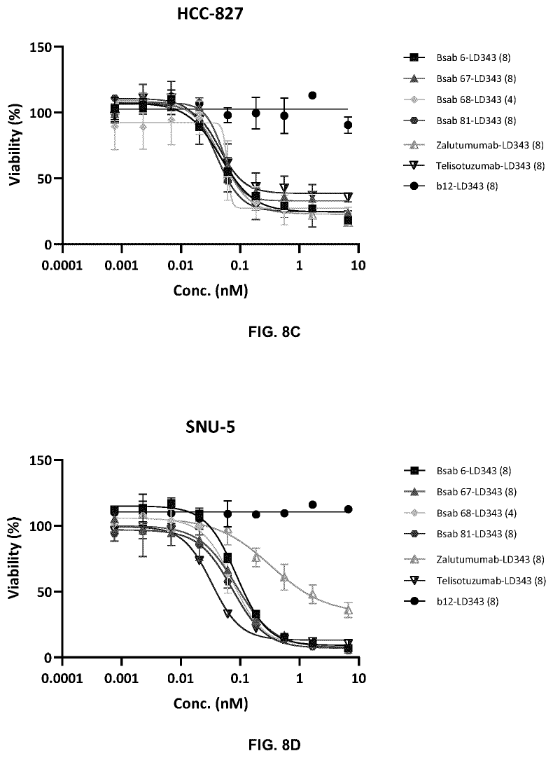

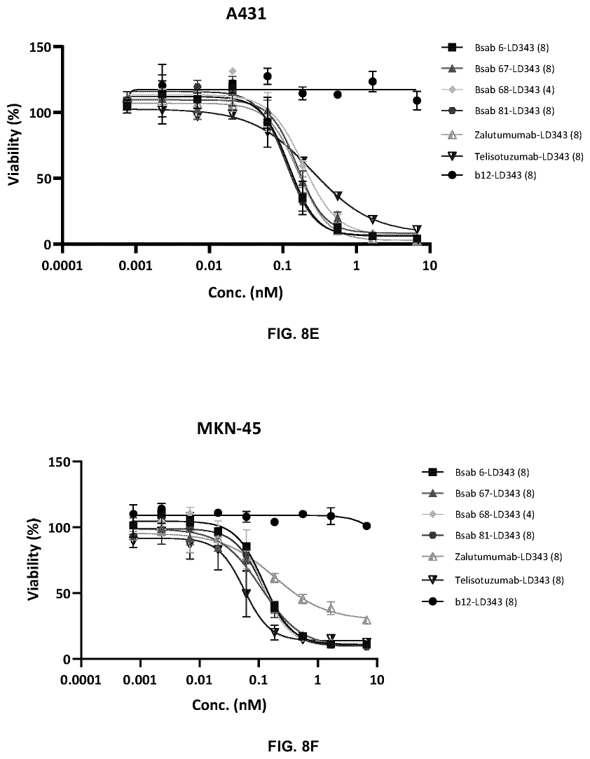

As shown in FIGS. 8A-8F, all bispecifics showed remarkable in vitro cytotoxicity in various cell lines conjugated with LD343.

### Example 17: Anti-Proliferation Effect of Bsab 67

Inhibition of EGFR-dependent cell growth was assessed by measuring viability of A431 cells (ATCC, Cat #CRL-1555). Cells were plated at 5000 cells/well in 96-well plates (Nunc) and starved for 24 h. Bsab 67 was added to cells and incubated for 96 h at 37° C. The number of viable cells were detected by adding 100 μL/well CellTiter-Glo® reagent (Promega), luminescence intensity was read by plate reader (Molecular Devices). IC₅₀ values were calculated by GraphPad Prism software.

Inhibition of cMET-dependent cell growth was assessed by measuring viability of SNU-5 cells (ATCC, Cat #CRL-5973). Cells were plated at 5000 cells/well in 96-well plates (Nunc) and starved for 24 h. Bsab 67 was added to cells and incubated for 96 h at 37° C. The number of viable cells were detected by adding 100 μL/well CellTiter-Glo® reagent (Promega), luminescence intensity was read by plate reader (Molecular Devices). IC₅₀ values were calculated by GraphPad Prism software.

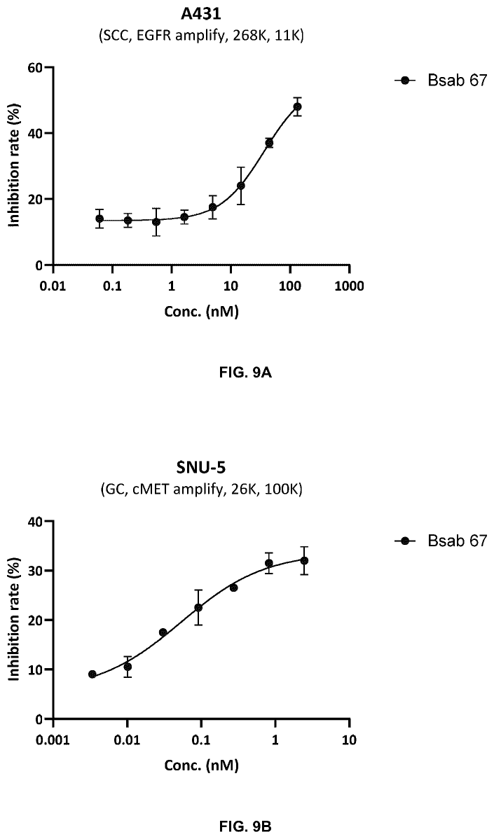

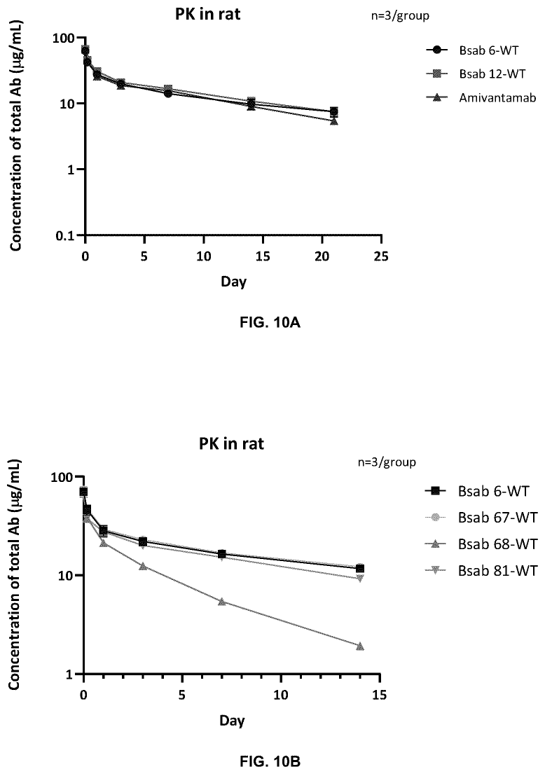

As shown in FIGS. 9A-9B, Bsab 67 produced robust anti-proliferation effects on tumor cell lines.

### Example 18: PK of Bispecific Antibodies and Bispecific Antibodies Conjugated with LD038 (8) or LD343 (8)

Bsab 6-WT, Bsab 12-WT and Amivantamab were intravenously administered at 3 mg/kg to male Sprague Dawley rats (n=3 per group). Jugular vein blood was sampled from each rat at 10 min, 4 h, 1 d, 3 d, 7 d, 14 d and 21 d post dosing. Total antibody (TAb) concentration of Bsab 6-WT and Bsab 12-WT in plasma was captured by human cMET protein and detected by goat anti-human IgG Fc (HRP), calculated using GraphPad Prism 8.0 software.

Bsab 6-WT, Bsab 67-WT, Bsab 68-WT and Bsab 81-WT were intravenously administered at 3 mg/kg to male Sprague Dawley rats (n=3 per group). Jugular vein blood was sampled from each rat at 10 min, 4 h, 1 d, 3 d, 7 d, 14 d and 21 d post dosing. Total antibody (TAb) concentration of Bsab 6-WT, Bsab 67-WT, Bsab 68-WT and Bsab 81-WT in plasma was captured by human cMET protein and detected by goat anti-human IgG Fc (HRP), calculated using GraphPad Prism 8.0 software.

Bsab 67, Bsab 81, Bsab 67-LD038 (8), Bsab 67-LD343 (8), Bsab 81-LD038 (8) and Bsab 81-LD343 (8) were intravenously administered at 3 mg/kg to male Sprague Dawley rats (n=3 per group). Jugular vein blood was sampled from each rat at 10 min, 4 h, 1 d, 3 d, 7 d, 14 d and 21 d post dosing. Total antibody (TAb) concentration of Bsab 67, Bsab 81, Bsab 67-LD038 (8), Bsab 67-LD343 (8), Bsab 81-LD038 (8) and Bsab 81-LD343 (8) in plasma was captured by human cMET protein and detected by goat anti-human IgG Fc (HRP), calculated using GraphPad Prism 8.0 software.

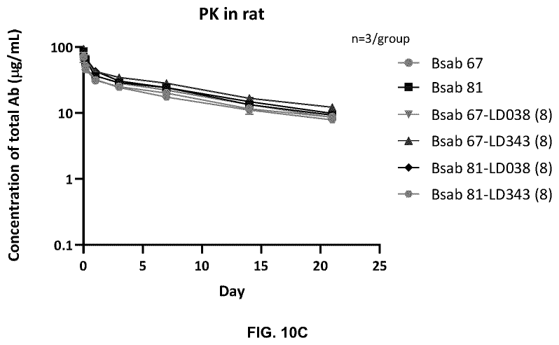

As shown in FIGS. 10A-10C, Bsab 67 and Bsab 81 wild type retained similar PK when conjugated with both LD038 (8) or LD343 (8), and Bsab 67 and Bsab 81 retained similar PK when conjugated with both LD038 (8) or LD343 (8).

### Example 19: In Vivo Efficacy of Bispecific ADCs

The in vivo anti-tumor activities of Telisotuzumab vedotin, MRG003 vedotin, Zalutumumab-LD038 (8), Telisotuzumab-LD038 (8), Bsab 6-LD038 (8), Bsab 6-LD343 (8), Bsab 12-LD038 (8), Bsab 12-LD343 (8), b12-LD038 (8), b12-LD343 (8) and Amivantamab were evaluated in EBC-1 and NCI-H1975 xenograft models.

EBC-1 (EK-Bioscience) tumor model was established by injecting 3×10⁶ cells suspended in 0.1 mL PBS. 7 days after tumor inoculation, mice with average tumor size ˜134 mm³ were selected and assigned into 14 groups using stratified randomization (n=6 per group) based upon their tumor volumes. Mice were treated with a single (on day 0) intravenous injection of Telisotuzumab vedotin at 2.5 mg/kg, MRG003 vedotin at 2.5 mg/kg, Zalutumumab-LD038 (8) at 5 mg/kg, Telisotuzumab-LD038 (8) at 5 mg/kg, Zalutumumab-LD038 (8)/Telisotuzumab-LD038 (8) at 2.5/2.5 mg/kg, Bsab 6-LD038 (8) at 5 mg/kg, Bsab 6-LD343 (8) at 1.25 mg/kg, Bsab 12-LD038 (8) at 5 mg/kg, Bsab 12-LD343 (8) at 1.25 mg/kg, b12-LD038 (8)) at 5 mg/kg, b12-LD343 (8) at 1.25 mg/kg, or Amivantamab at 10, 5 mg/kg.

NCI-H1975 (Procell) tumor model was established by injecting 5×10⁶ cells suspended in 0.1 mL PBS mixed with Matrigel (1:1). 21 days after tumor inoculation, mice with average tumor size ˜119 mm³ were selected and assigned into 14 groups using stratified randomization (n=5 per group) based upon their tumor volumes. Mice were treated with a single (on day 0) intravenous injection of Telisotuzumab vedotin at 2.5 mg/kg, MRG003 vedotin at 2.5 mg/kg, Zalutumumab-LD038 (8) at 5 mg/kg, Telisotuzumab-LD038 (8) at 5 mg/kg, Zalutumumab-LD038 (8)/Telisotuzumab-LD038 (8) at 2.5/2.5 mg/kg, Bsab 6-LD038 (8) at 5 mg/kg, Bsab 6-LD343 (8) at 1.25 mg/kg, Bsab 12-LD038 (8) at 5 mg/kg, Bsab 12-LD343 (8) at 1.25 mg/kg, b12-LD038 (8)) at 5 mg/kg, b12-LD343 (8) at 1.25 mg/kg, or Amivantamab at 10, 5 mg/kg.

The tumor size and body weight were measured as described before. Animal body weight was monitored as an indirect measure of tolerability. No mice showed significant weight loss in any of the treatment groups. There were no morbidity and deaths during the treatment duration.

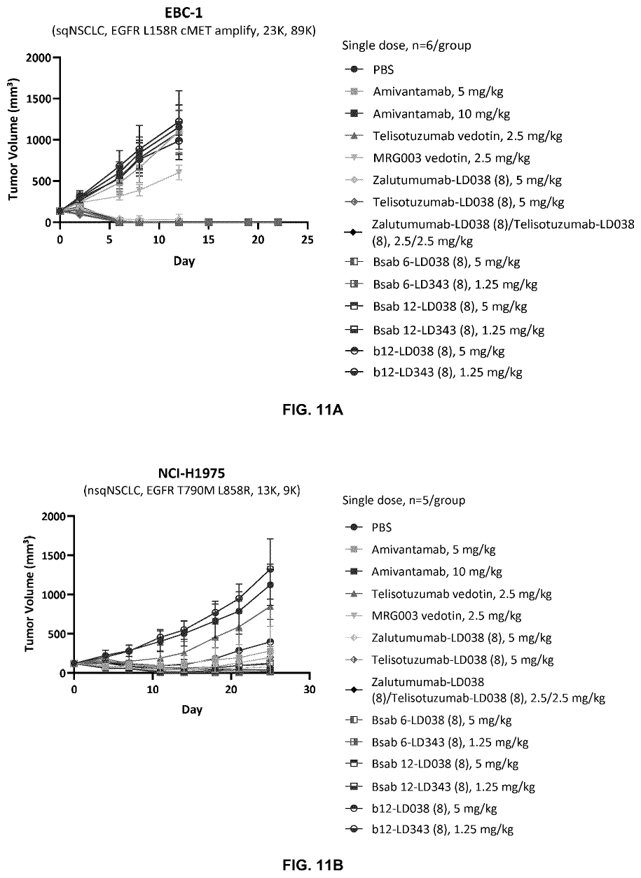

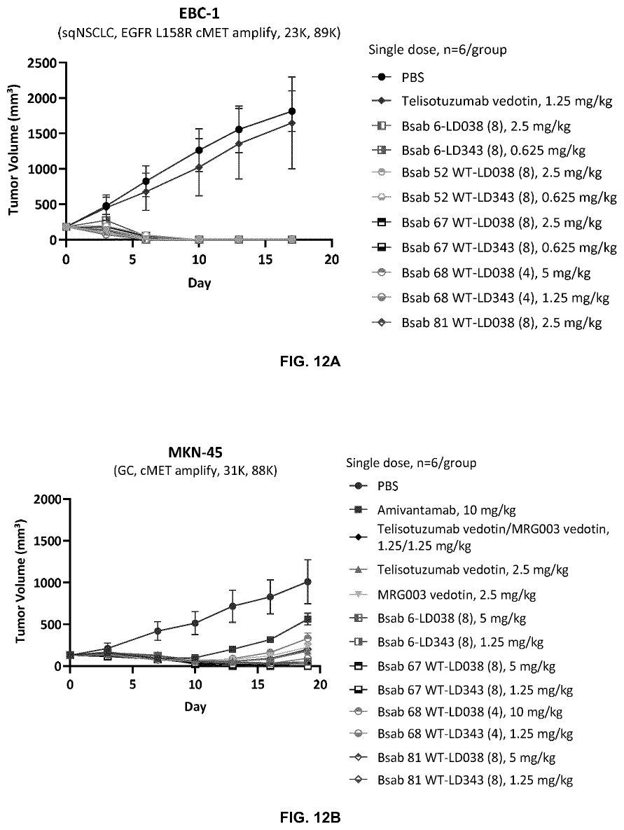

As shown in FIGS. 11A-11B, Bsab 6 and Bsab 12 showed great anti-tumor activity compared with parent ADC and amivantamab.

### Example 20: In Vivo Efficacy of Bispecific ADCs

The in vivo anti-tumor activities of Telisotuzumab vedotin, MRG003 vedotin, Bsab 6-LD038 (8), Bsab 6-LD343 (8), Bsab 52 WT-LD038 (8), Bsab 52 WT-LD343 (8), Bsab 67 WT-LD038 (8), Bsab 67 WT-LD343 (8), Bsab 68 WT-LD038 (4), Bsab 68 WT-LD343 (4), Bsab 81 WT-LD038 (8), Bsab 81 WT-LD343 (8), and Amivantamab were evaluated in EBC-1, MKN-45, SNU-5 and NCI-H1975 xenograft models.

EBC-1 (EK-Bioscience) tumor model was established by injecting 3×10⁶ cells suspended in 0.1 mL PBS. 8 days after tumor inoculation, mice with average tumor size ˜180 mm³ were selected and assigned into 11 groups using stratified randomization (n=6 per group) based upon their tumor volumes. Mice were treated with a single (on day 0) intravenous injection of Telisotuzumab vedotin at 1.25 mg/kg, Bsab 6-LD038 (8) at 2.5 mg/kg, Bsab 6-LD343 (8) at 0.625 mg/kg, Bsab 52 WT-LD038 (8) at 2.5 mg/kg, Bsab 52 WT-LD343 (8) at 0.625 mg/kg, Bsab 67 WT-LD038 (8) at 2.5 mg/kg, Bsab 67 WT-LD343 (8) at 0.625 mg/kg, Bsab 68 WT-LD038 (4) at 5 mg/kg, Bsab 68 WT-LD343 (4) at 1.25 mg/kg, Bsab 81 WT-LD038 (8) at 2.5 mg/kg, or Amivantamab at 10 mg/kg.

MKN-45 (Procell) tumor model was established by injecting 5×10⁶ cells suspended in 0.1 mL PBS mixed with Matrigel (1:1). 6 days after tumor inoculation, mice with average tumor size ˜131 mm³ were selected and assigned into 13 groups using stratified randomization (n=6 per group) based upon their tumor volumes. Mice were treated with a single (on day 0) intravenous injection of Telisotuzumab vedotin at 2.5 mg/kg, MRG003 vedotin at 2.5 mg/kg, Telisotuzumab vedotin/MRG003 vedotin at 1.25/1.25 mg/kg, Bsab 6-LD038 (8) at 5 mg/kg, Bsab 6-LD343 (8) at 1.25 mg/kg, Bsab 52 WT-LD038 (8) at 5 mg/kg, Bsab 52 WT-LD343 (8) at 1.25 mg/kg, Bsab 67 WT-LD038 (8) at 2.5 mg/kg, Bsab 67 WT-LD343 (8) at 1.25 mg/kg, Bsab 68 WT-LD038 (4) at 10 mg/kg, Bsab 68 WT-LD343 (4) at 1.25 mg/kg, Bsab 81 WT-LD038 (8) at 5 mg/kg, Bsab 81 WT-LD343 (8) at 1.25 mg/kg, or Amivantamab at 10 mg/kg.

SNU-5 (COBIOER) tumor model was established by injecting 5×10⁶ cells suspended in 0.1 mL PBS. 8 days after tumor inoculation, mice with average tumor size ˜131 mm³ were selected and assigned into 13 groups using stratified randomization (n=6 per group) based upon their tumor volumes. Mice were treated with a single (on day 0) intravenous injection of Telisotuzumab vedotin at 2.5 mg/kg, MRG003 vedotin at 2.5 mg/kg, Telisotuzumab vedotin/MRG003 vedotin at 1.25/1.25 mg/kg, Bsab 6-LD038 (8) at 5 mg/kg, Bsab 6-LD343 (8) at 1.25 mg/kg, Bsab 52 WT-LD038 (8) at 5 mg/kg, Bsab 52 WT-LD343 (8) at 1.25 mg/kg, Bsab 67 WT-LD038 (8) at 2.5 mg/kg, Bsab 67 WT-LD343 (8) at 1.25 mg/kg, Bsab 68 WT-LD038 (4) at 10 mg/kg, Bsab 68 WT-LD343 (4) at 1.25 mg/kg, Bsab 81 WT-LD038 (8) at 5 mg/kg, Bsab 81 WT-LD343 (8) at 1.25 mg/kg, or Amivantamab at 10 mg/kg.

NCI-H1975 (Procell) tumor model was established by injecting 5×10⁶ cells suspended in 0.1 mL PBS mixed with Matrigel (1:1). 21 days after tumor inoculation, mice with average tumor size ˜199 mm³ were selected and assigned into 11 groups using stratified randomization (n=6 per group) based upon their tumor volumes. Mice were treated with a single (on day 0) intravenous injection of Telisotuzumab vedotin at 2.5 mg/kg, MRG003 vedotin at 2.5 mg/kg, Telisotuzumab vedotin/MRG003 vedotin at 2.5/2.5 mg/kg, Bsab 6-LD038 (8) at 5 mg/kg, Bsab 6-LD343 (8) at 1.25 mg/kg, Bsab 67 WT-LD038 (8) at 2.5 mg/kg, Bsab 67 WT-LD343 (8) at 1.25 mg/kg, Bsab 81 WT-LD038 (8) at 5 mg/kg, Bsab 81 WT-LD343 (8) at 1.25 mg/kg, or Amivantamab at 10 mg/kg.

The tumor size and body weight were measured as described before. Animal body weight was monitored as an indirect measure of tolerability. No mice showed significant weight loss in any of the treatment groups. There were no morbidity and deaths during the treatment duration.

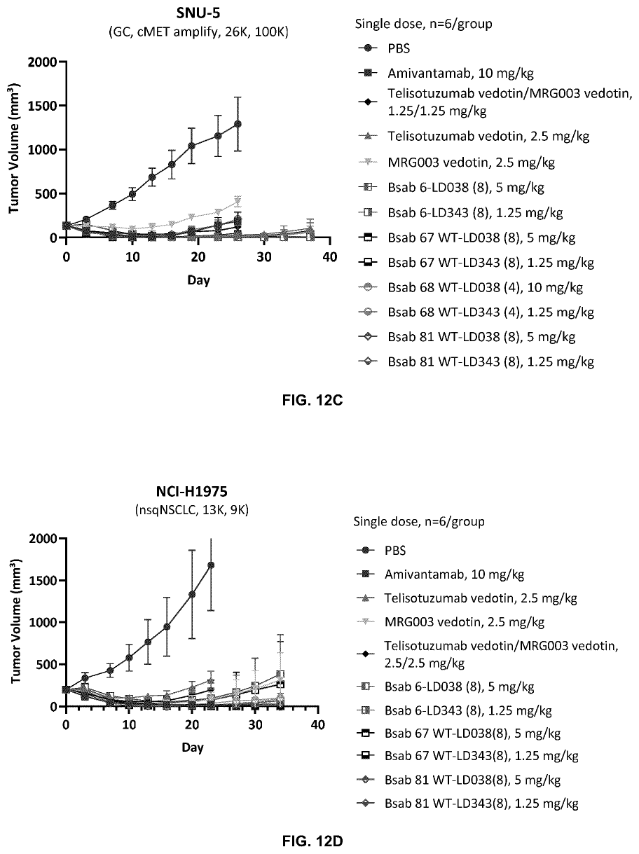

As shown in FIGS. 12A-12D, All bispecific ADCs showed great anti tumor activity, Bsab 68 showed lesser activity probably due to worse PK.

### Example 21: In Vivo Efficacy of Bispecific ADCs

The in vivo anti-tumor activities of Bsab 67 WT, Bsab 67, Bsab 81, Bsab 67 WT-LD038 (8), Bsab 67 WT-LD343 (8), Bsab 67-LD038 (8), Bsab 67-LD343 (8), Bsab 81 WT-LD038 (8), Bsab 81 WT-LD343 (8), and Amivantamab were evaluated in NCI-N87 and MKN-45 xenograft models.

NCI-N87 tumor model was established by injecting 5×10⁶ cells suspended in 0.1 mL PBS. 3 days after tumor inoculation, mice with average tumor size ˜154 mm³ were selected and assigned into 8 groups using stratified randomization (n=6 per group) based upon their tumor volumes. Mice were treated with a single (on day 0) intravenous injection of Bsab 67-LD038 (8) at 5 mg/kg, Bsab 67-LD343 (8) at 1.25 mg/kg, Bsab 67 WT-LD038 (8) at 5 mg/kg, Bsab 67 WT-LD343 (8) at 1.25 mg/kg, Bsab 81 WT-LD038 (8) at 5 mg/kg, Bsab 81 WT-LD343 (8) at 1.25 mg/kg, or Amivantamab at 10 mg/kg.

MKN-45 (Procell) tumor model was established by injecting 5×10⁶ cells suspended in 0.1 mL PBS mixed with Matrigel (1:1). 14 days after tumor inoculation, mice with average tumor size ˜131 mm³ were selected and assigned into 9 groups using stratified randomization (n=6 per group) based upon their tumor volumes. Mice were treated with a single (on day 0) intravenous injection of Bsab 67-LD038 (8) at 5 mg/kg, Bsab 67-LD343 (8) at 1.25 mg/kg, Bsab 67 WT-LD038 (8) at 5 mg/kg, Bsab 67 WT-LD343 (8) at 1.25 mg/kg, Bsab 67 WT at 10 mg/kg, Bsab 67 at 10 mg/kg, Bsab 81 at 10 mg/kg, or Amivantamab at 10 mg/kg.

The tumor size and body weight were measured as described before. Animal body weight was monitored as an indirect measure of tolerability. No mice showed significant weight loss in any of the treatment groups. There were no morbidity and deaths during the treatment duration.

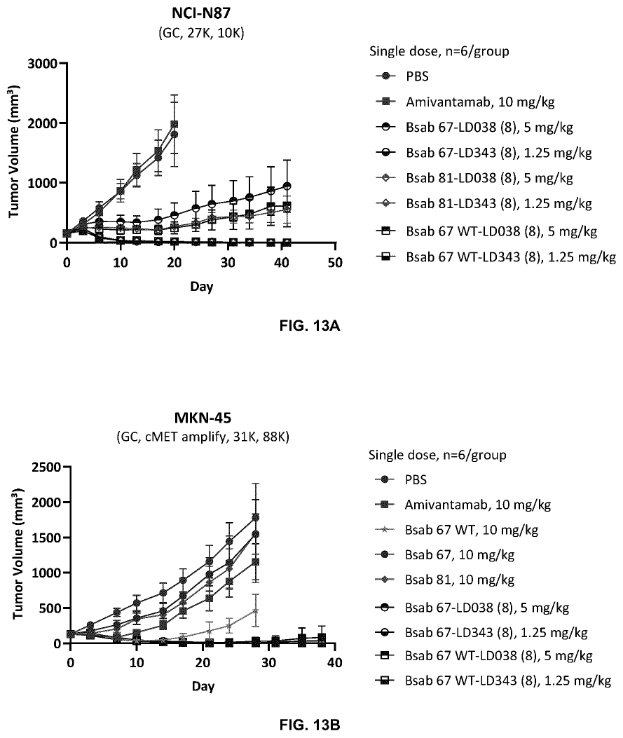

As shown in FIGS. 13A-13B, ab 67 WT showed stronger anti tumor activity than Bsab 67 or 81, possibly due to ADC effect; Bsab 67 WT conjugated with LD038 (8) and LD343 (8) showed stronger efficacy compared with Bsab 67 and 81 conjugated with similar LD in NCI-N87 model, but similar efficacy in MKN-45.

### Example 22: In Vivo Efficacy of Bispecific ADCs

The in vivo anti-tumor activities of Bsab 67-LD038 (8), Bsab 67-LD343 (8), Bsab 81-LD038 (8), Bsab 81-LD343 (8) and Amivantamab were evaluated in TE4 xenograft model.

TE4 (COBIOER) tumor model was established by injecting 5×10⁶ cells suspended in 0.1 mL PBS. 11 days after tumor inoculation, mice with average tumor size ˜127 mm³ were selected and assigned into 6 groups using stratified randomization (n=4 per group) based upon their tumor volumes. Mice were treated with a single (on day 0) intravenous injection of Bsab 67-LD038 (8) at 5 mg/kg, Bsab 67-LD343 (8) at 1.25 mg/kg, Bsab 67 WT-LD038 (8) at 5 mg/kg, Bsab 67 WT-LD343 (8) at 1.25 mg/kg, Bsab 81 WT-LD038 (8) at 5 mg/kg, Bsab 81 WT-LD343 (8) at 1.25 mg/kg, or Amivantamab at 10 mg/kg.

The tumor size and body weight were measured as described before. Animal body weight was monitored as an indirect measure of tolerability. No mice showed significant weight loss in any of the treatment groups. There were no morbidity and deaths during the treatment duration.

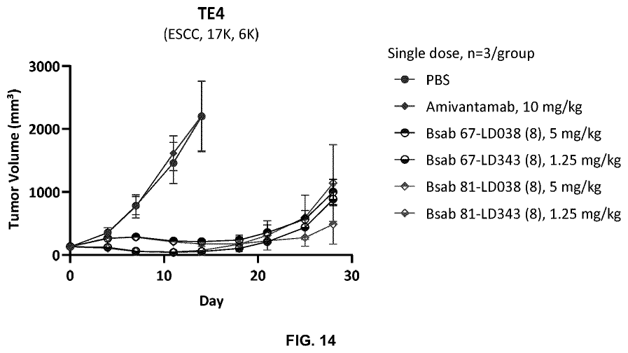

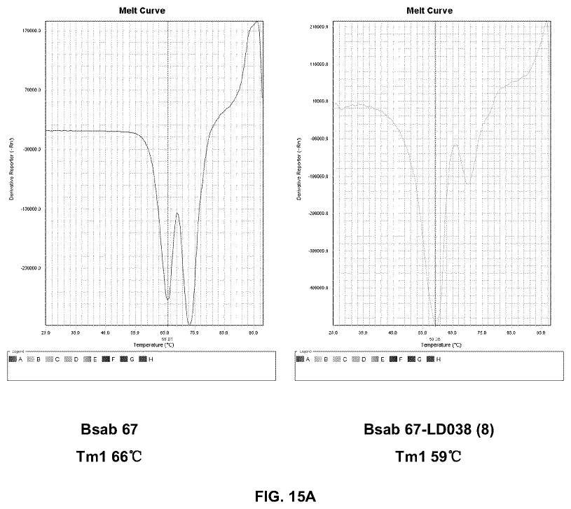

As shown in FIG. 14, Bsab 67 and 81 showed similar anti tumor activity conjugated with both LD038 (8) and LD343 (8).

### Example 23: Heat-Stability of Bsab 67-LD038(8)

Differential scanning fluorimetry (DSF) experiments were performed using Real-Time PCR Detection System (Applied Biosystems 7500). Proteins were mixed with SYPRO Orange fluorescent dye (MERCK, S5692) and diluted to 0.5 mg/mL in 20 mM histidine buffer (pH6.0). The final concentration of SYPRO Orange was 10×. Proteins were heated from 25 to 95° C., using a heating rate of 1° C./min. A fluorescence measurement was taken every 30 s. Melting temperatures were calculated using the instrument software.

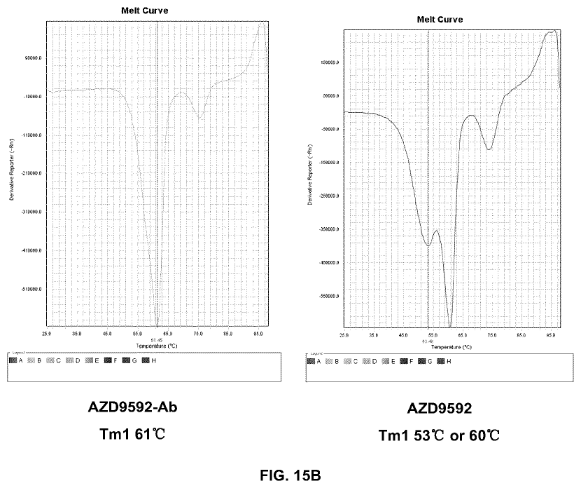

As shown in FIGS. 15A-15B, Bsab 67-LD038(8) is more heat-stable than AZD9592.

### Example 24: Hydrophilicity of Bsab 67-LD038(8)

The hydrophobicity of test articles was measured in Waters® e2695 Separations Module with UV detection at 280 nm using TSKgel Butyl-NPR column (Tosoh Bioscience, TO0042168). The mobile phase A is 50 mM phosphate buffer, 1.5 M ammonium sulfate, pH 7.0. Mobile phase B is 50 mM phosphate buffer pH 7.0, 20% isopropanol. The flow rate is 0.5 mL/min at room temperature. Use Empower™3 SOFTWARE to analyze the peak retention time.

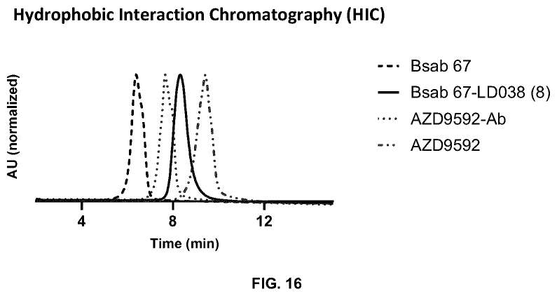

As shown in FIG. 16, Bsab 67-LD038(8) and its parent mAb are more hydrophilic than AZD9592 and its parent mAb.

### Example 25: Binding Specificity of Bsab 67-LD038 (8) and Bsab 67

Binding specificity was tested by ELISA according to the standard protocol. Specifically, 96-well plates were coated with 100 ng/well human EGFR, human HER2, human HER3, human HER4, human cMET, human Sema3A recombinant protein in PBS, incubated overnight at 4° C. Plates were washed twice by TBS+0.05% Tween20. 200 μL blocking buffer (2% BSA in PBS) was added to each well and incubated at 37° C. for 2 h. After wash, Serial diluted antibodies were added to the ELISA plate with 100 μL per well and incubated for 1 h at room temperature. Then plates were washed for 3 times. HRP conjugated anti-human Fc antibody solution (Abcam, ab98624) was added to the plate with 100 μL per well. The plates were incubated at room temperature for 1 h and then washed for 3 times. TMB solution was added to plates with 100 μL per well, placed at room temperature for 5-15 mins, then the stop solution (2 M H₂SO₄) was added with 50 μL per well. Finally, read the absorbance at A₄₅₀ and A₆₃₀.

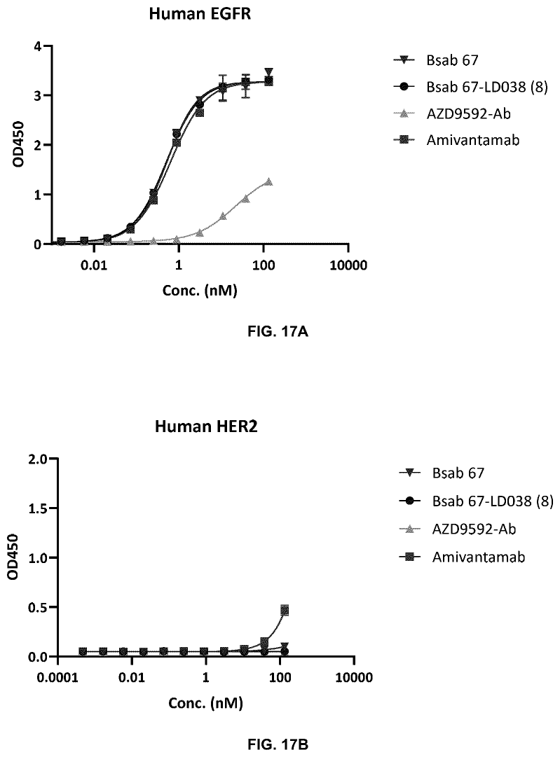

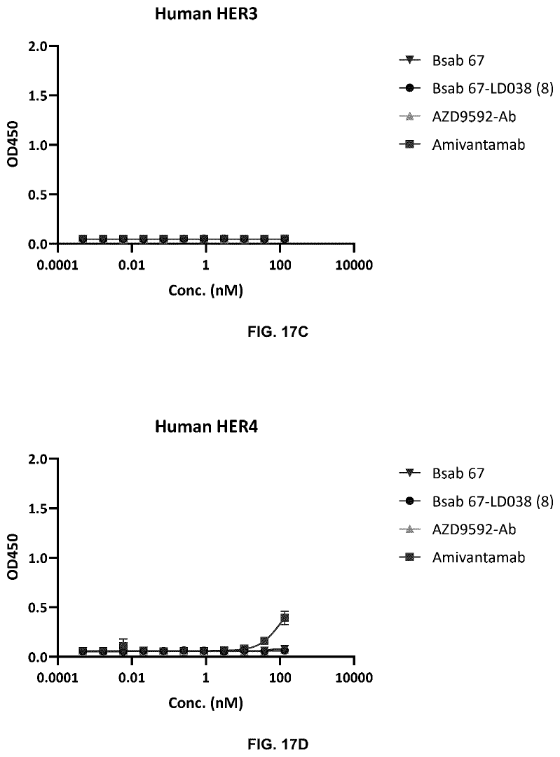

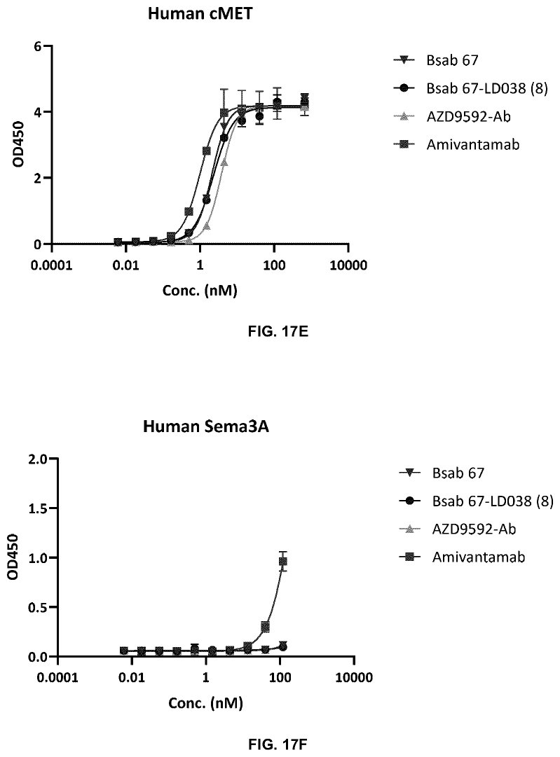

As shown in FIGS. 17A-17F, Bsab 67-LD038 (8) and Bsab 67 showed no binding to EGFR and cMET family members.

### Example 26: Binding Activity of Bsab 67-LD038 (8) and Bsab 67

EGFR and cMET co-expressed tumor cell lines including A431 (ATCC® CRL-1555), SW620 (ATCC® CCL-227), EBC-1 (JCRB JCRB0820, Provided by Shanghai EK-Bioscience), SNU-5 (ATCC® CRL-5973, Provided by COBIOER), CAL-27 (ATCC® CRL-2095, Provided by COBIOER), KYSE-30 (DSMZ ACC 351, Provided by COBIOER), MKN-45 (CL-0292, Provided by Procell), HT-29 (ATCC® HTB-38) were selected for evaluating antibody binding activity by flow cytometry.

A431, SW620, EBC-1 and CAL-27 were cultured with DMEM medium (Gibco, Cat #C11995500BT) containing 10% FBS. KYSE-30 and MKN-45 were cultured with RPMI 1640 (Gibco, Cat #11875093) containing 10% FBS. HT-29 was cultured with McCoy's 5a medium (Gibco, Cat #16600082) containing 10% FBS. SNU-5 was cultured with IMDM medium (Gibco, Cat #12440053) containing 10% FBS. Each antibody was incubated with different cell lines (1×10⁵ cells/well) for 30 mins in 0.2 mL FACS buffer (1×PBS with 0.1% BSA) at 4° C. After wash, cells were incubated with 100 μL PE-conjugated anti human Fc (Abcam, Ab98596, 1:500 dilution) for 30 mins at 4° C. After wash with PBS, cells were resuspended in FACS buffer and analyzed by flow cytometer (Beckman, CytoFLEX).

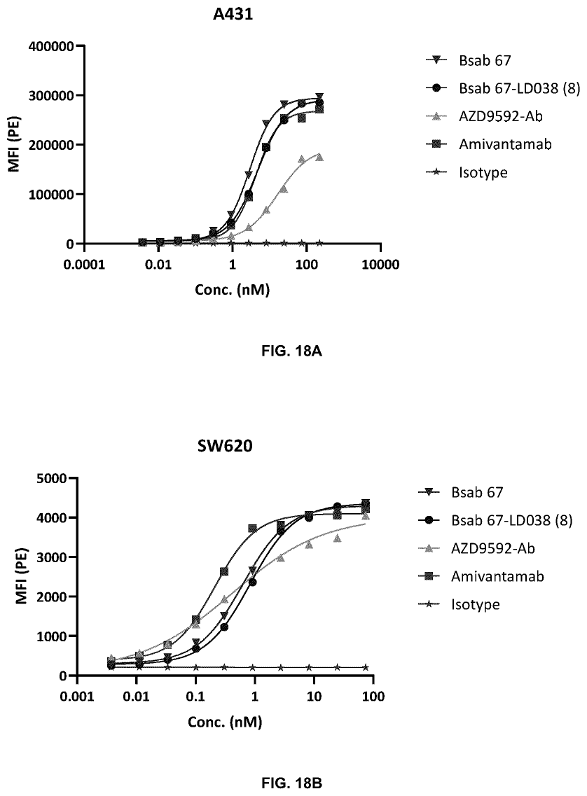

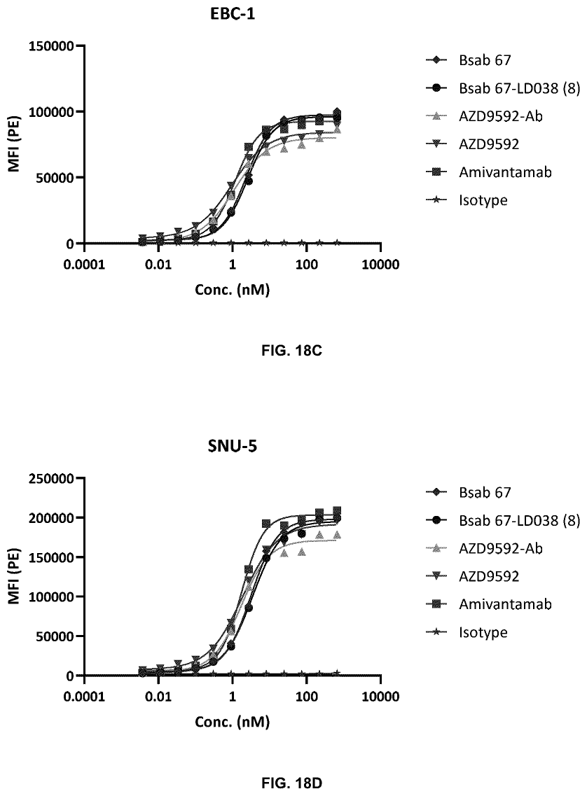

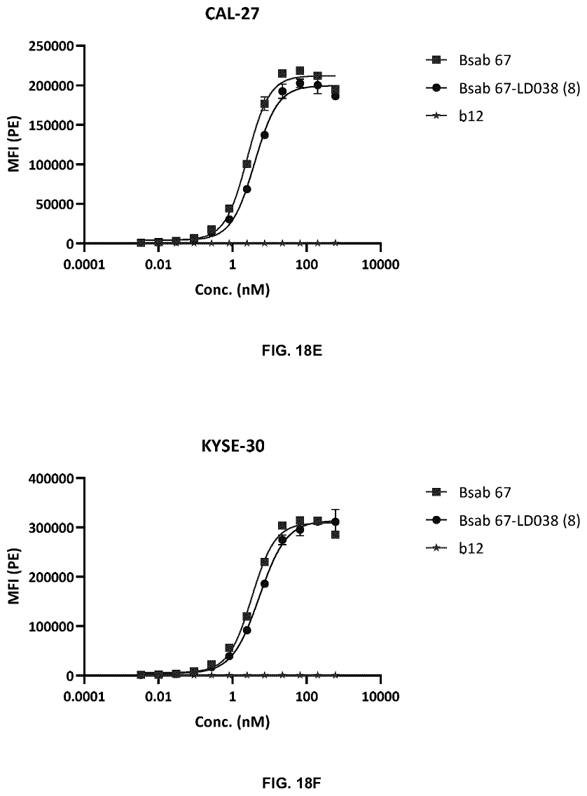

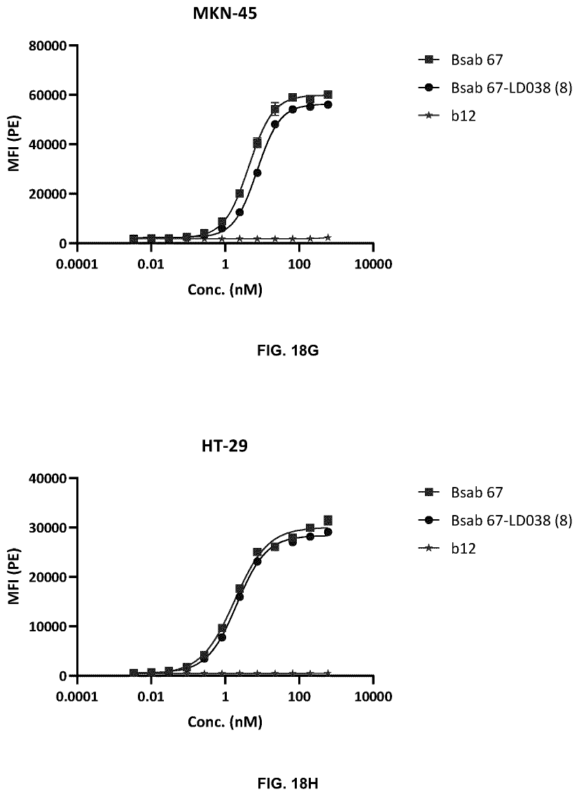

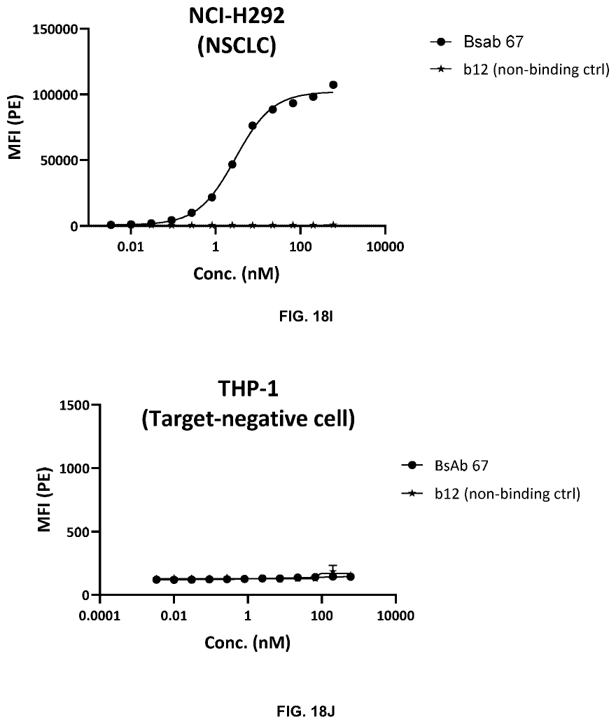

As shown in FIGS. 18A-18H, Bsab 67-LD038 showed stronger binding to tumor cells than benchmarks.

### Example 27: Binding Activity of Bsab 67

EGFR and cMET co-expressed tumor cell line NCI-H292 (ATCC® CRL-1848, Provided by COBIOER) and EGFR and c-MET negative cell line THP-1 (ATCC® TIB-202, Provided by COBIOER) were selected for evaluating antibody binding activity by flow cytometry. NCI-H292 and THP-1 were cultured with RPMI 1640 (Gibco, Cat #11875093) containing 10% FBS. Each antibody was incubated with different cell lines (1×10⁵ cells/well) for 30 min in 0.2 mL FACS buffer (1×PBS with 0.1% BSA) at 4° C. After wash, cells were incubated with 100 μL PE-conjugated anti human Fc (Abcam, Ab98596, 1:500 dilution) for 30 min at 4° C. After wash with PBS, cells were resuspended in FACS buffer and analyzed by flow cytometer (Beckman, CytoFLEX).

As shown in FIGS. 18I-18J, Bsab 67 showed stronger binding to tumor cells than benchmarks and showed no binding to target-negative cells.

### Example 28: Internalization of Bsab 67-LD038 (8) and Bsab 67

The internalization assay was conducted in time course. 1×10⁵ cells were incubated with 200 nM test articles for 30 mins at 4° C. in FACS buffer (1×PBS containing 0.1% BSA). Cells were washed at 4° C. to remove unbound material and kept on ice or shifted to 37° C. as needed. At progressive time points (0, 0.5, 2, 4 h), cells were stained with PE-conjugated anti-human Fc for 30 mins at 4° C. and analyzed by flow cytometry. Internalization rate was calculated by subtracting the mean fluorescence intensity (MFI) of cell surface-bound antibody at 37° C. at each timepoint from the MFI of cell surface-bound antibody at 4° C. at time 0, then divided by the MFI of cell surface-bound antibody at 4° C. at time 0.

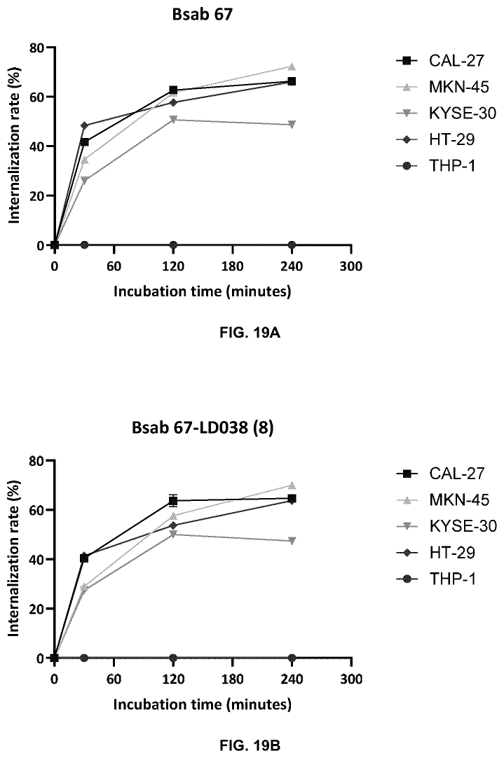

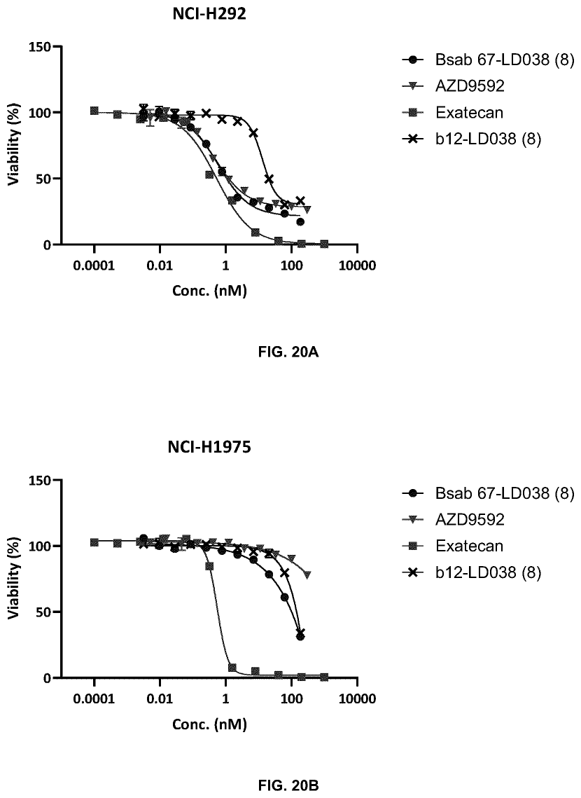

As shown in FIGS. 19A-19B, Bsab 67 and Bsab 67-LD038 (8) show robust internalization in various tumor cell lines.

### Example 29: Cytotoxicity of Bsab 67-LD038 (8)

Cells were harvested and seeded into 96-well plates (Coring, 3917) for overnight culture. In the next day, serial diluted test articles (Bsab 67-LD038 (8), AZD9592, Exatecan or b12-LD038 (8)) were added to cells and the incubation was lasting for 144 h at 37° C. After that, 40 μL CellTiter-Glo (Promega) was added to each well and incubated for 5 mins at room temperature. Luciferase readings were collected by microplate reader. All readings were normalized as percentage of viable cells in the untreated control wells and the IC₅₀ values were calculated by GraphPad Prism software.

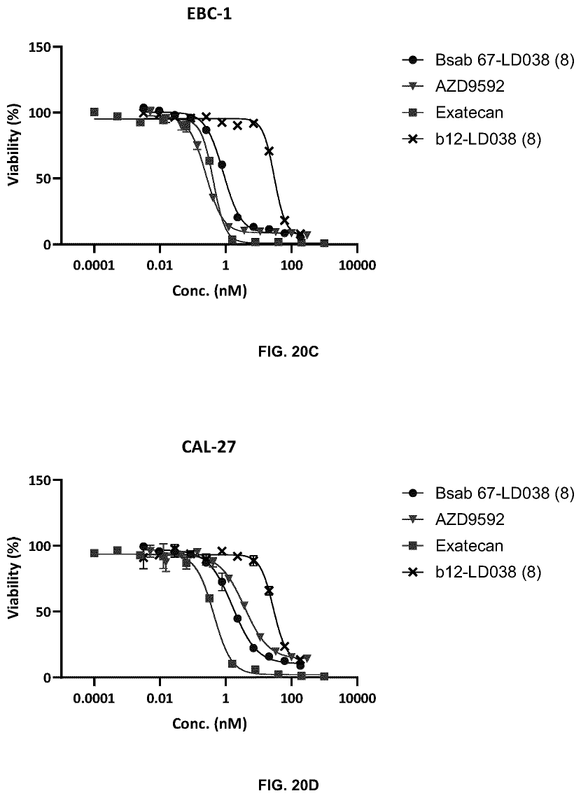

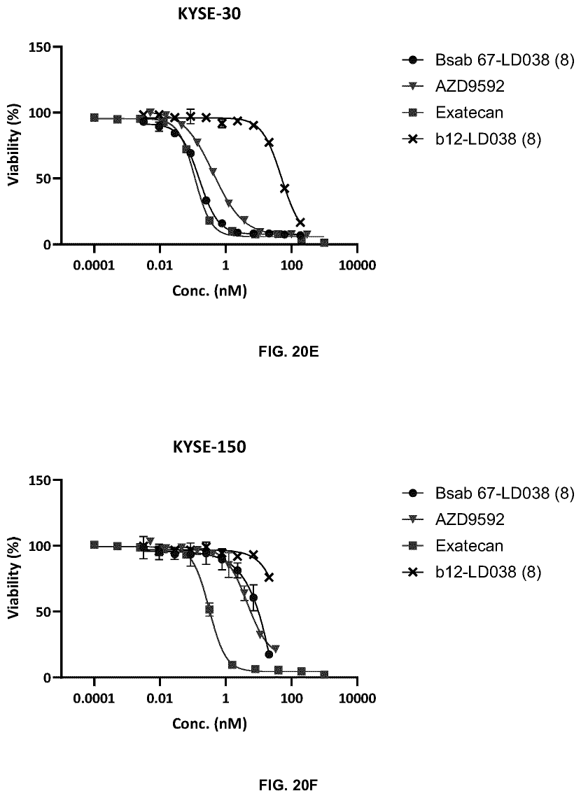

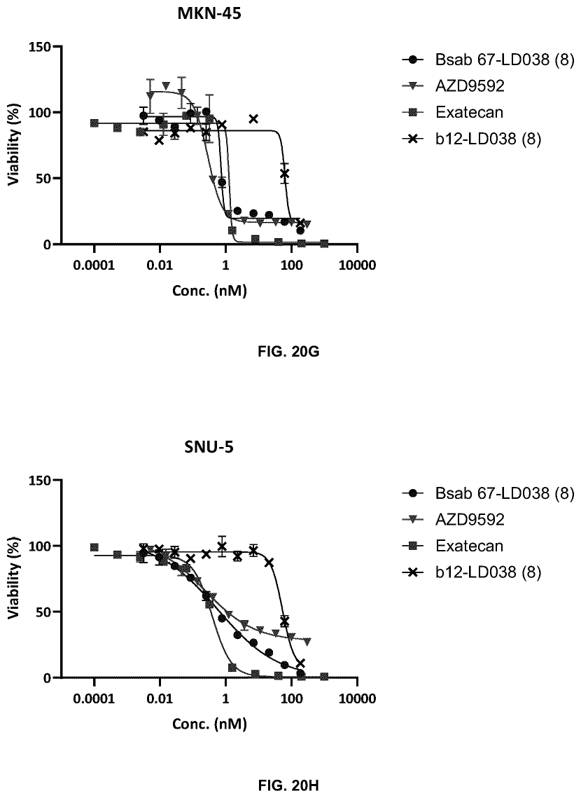

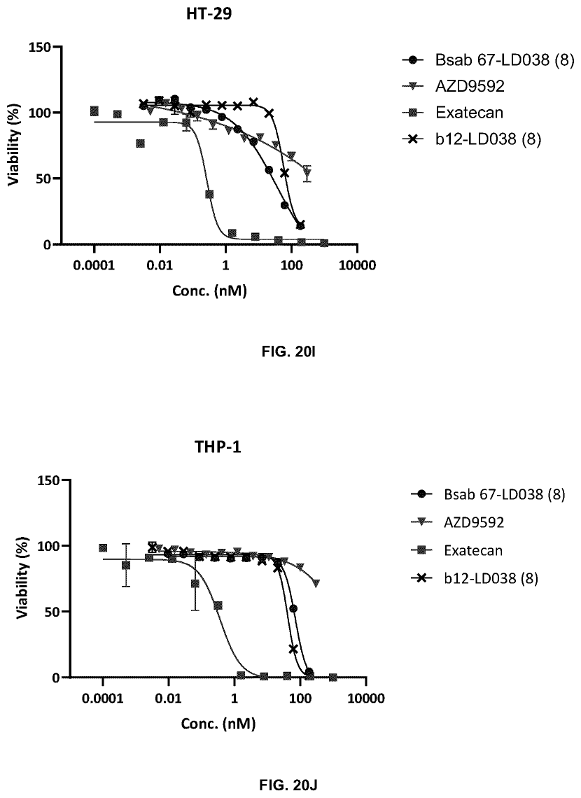

As shown in FIGS. 20A-20J, Bsab 67-LD038 (8) shows superior cytotoxicity than AZD9592 in various tumor cell lines.

### Example 30: Bystander Effect of Bsab 67-LD038 (8)

Target positive cells KYSE-30 and EGFR & cMET negative cells THP-1-Luc were inoculated into 96-well plates (Nunc) with prespecified densities (KYSE-30:1000 cells/well; THP-1-Luc:1000 cells/well). In the next day, Bsab 67-LD038 (8) was added and incubated with cells for 144 h at 37° C. Viable cells were detected by adding 80 μL/well Bio-Lite® reagent (Promega) and luminescence intensity was read by plate reader (Molecular Devices SpectraMax M5). Data was plotted as raw luminescence units (RLU) against the logarithm of Bsab 67-LD038 (8) molar concentration. IC₅₀ values were determined by GraphPad Prism software.

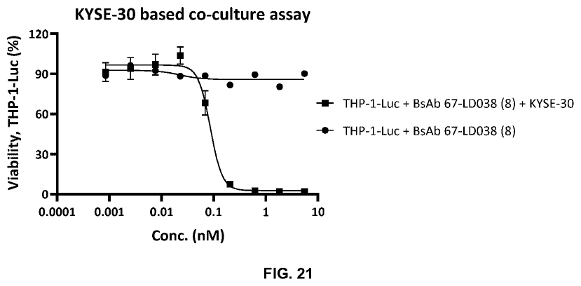

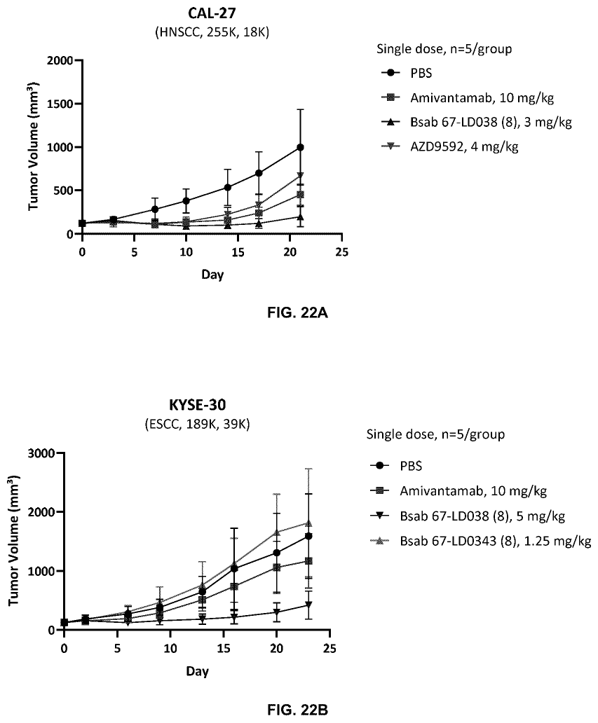

As shown in FIG. 21, Bsab 67-LD038 (8) shows great bystander effect.

### Example 31: In Vivo Efficacy of Bsab 67-LD038 (8)

The in vivo anti-tumor activities of Amivantamab and Bsab 67 analogue-LD038 (8) were evaluated in CAL-27, KYSE-30, MDA-MB-468, FADU, MKN-45 and SNU-5 xenograft models.

CAL-27 (COBIOER) tumor model was established by injecting 5×10⁶ cells suspended in 0.1 mL PBS. 34 days after tumor inoculation, mice with average tumor size ˜121 mm³ were selected and assigned into 5 groups using stratified randomization (n=5 per group) based upon their tumor volumes. Mice were treated with a single (on day 0) intravenous injection of AZD9592 at 4 mg/kg, Bsab 67-LD038 (8) at 3 mg/kg, or Amivantamab at 10 mg/kg.

KYSE-30 (COBIOER) tumor model was established by injecting 5×10⁶ cells suspended in 0.1 mL PBS. 20 days after tumor inoculation, mice with average tumor size ˜126 mm³ were selected and assigned into 4 groups using stratified randomization (n=5 per group) based upon their tumor volumes. Mice were treated with a single (on day 0) intravenous injection of Bsab 67-LD038 (8) at 5 mg/kg, or Amivantamab at 10 mg/kg.

MDA-MB-468 (COBIOER) tumor model was established by injecting 3×10⁶ cells suspended in 0.1 mL PBS mixed with Matrigel (1:1). 14 days after tumor inoculation, mice with average tumor size ˜131 mm³ were selected and assigned into 3 groups using stratified randomization (n=5 per group) based upon their tumor volumes. Mice were treated with a single (on day 0) intravenous injection of Bsab 67-LD038 (8) at 5 mg/kg.

FADU (COBIOER) tumor model was established by injecting 5×10⁶ cells suspended in 0.1 mL PBS. 6 days after tumor inoculation, mice with average tumor size ˜123 mm³ were selected and assigned into 2 groups using stratified randomization (n=6 per group) based upon their tumor volumes. Mice were treated with a single (on day 0) intravenous injection of Bsab 67-LD038 (8) at 3.5 mg/kg.

MKN-45 (Procell) tumor model was established by injecting 5×10⁶ cells suspended in 0.1 mL PBS mixed with Matrigel (1:1). 7 days after tumor inoculation, mice with average tumor size ˜132 mm³ were selected and assigned into 5 groups using stratified randomization (n=6 per group) based upon their tumor volumes. Mice were treated with a single (on day 0) intravenous injection of AZD9592 at 4 mg/kg, Bsab 67-LD038 (8) at 3 mg/kg, or Amivantamab at 10 mg/kg.

SNU-5 (COBIOER) tumor model was established by injecting 5×10⁶ cells suspended in 0.1 mL PBS. 8 days after tumor inoculation, mice with average tumor size ˜131 mm³ were selected and assigned into 2 groups using stratified randomization (n=6 per group) based upon their tumor volumes. Mice were treated with a single (on day 0) intravenous injection of Bsab 67-LD038 (8) at 2 mg/kg.

The tumor size and body weight were measured as described before. Animal body weight was monitored as an indirect measure of tolerability. No mice showed significant weight loss in any of the treatment groups. There were no morbidity and deaths during the treatment duration.

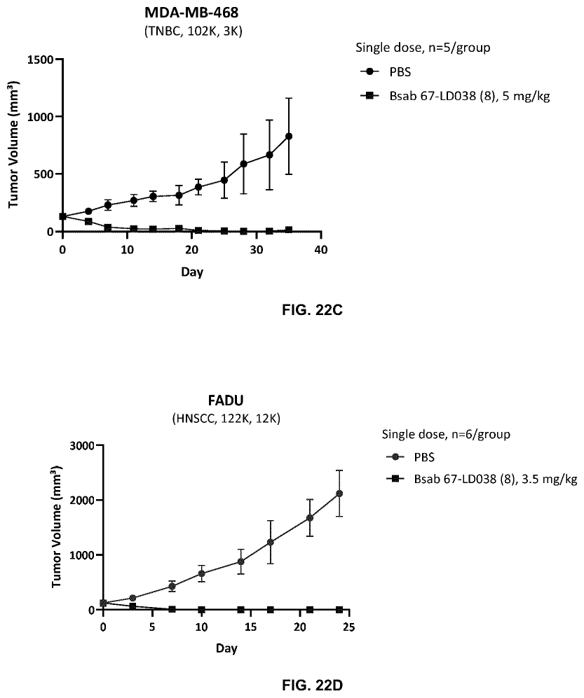

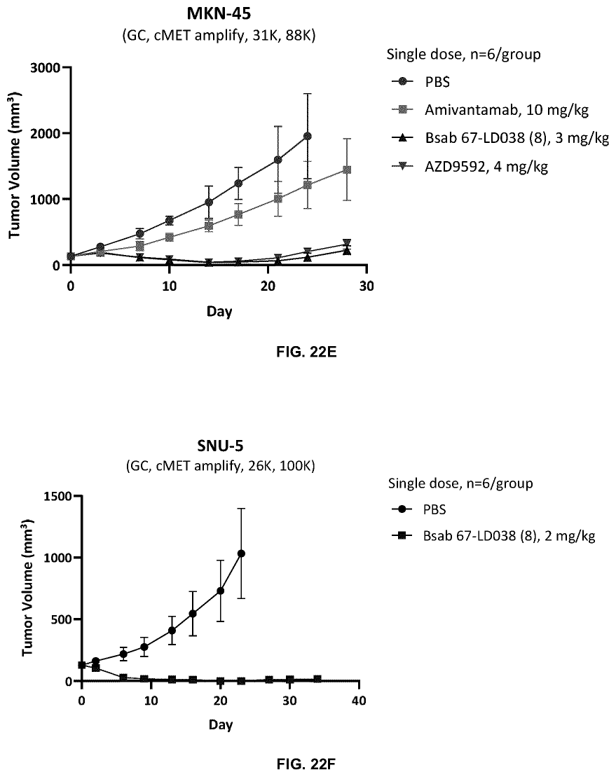

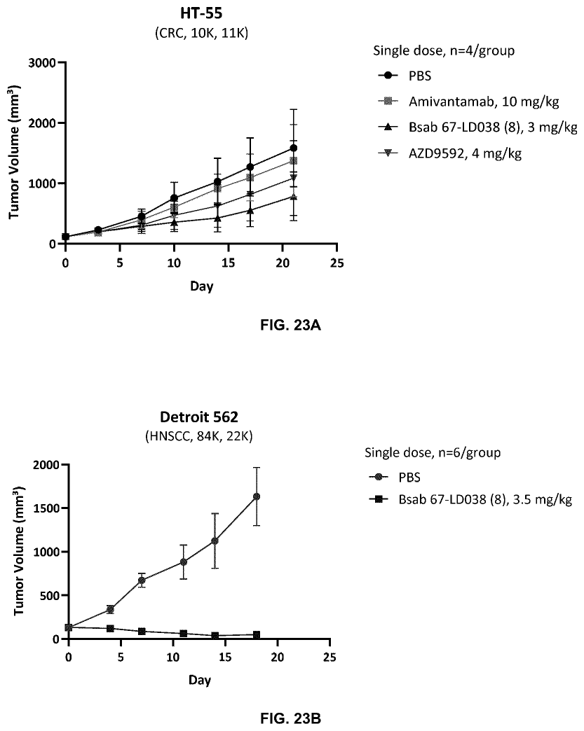

As shown in FIGS. 22A-22F, Bsab 67-LD038 (8) inhibits tumor cell lines with actionable genomic alterations in vivo.

### Example 32: In Vivo Efficacy of Bsab 67-LD038 (8)

The in vivo anti-tumor activities of Amivantamab, Bsab 67-LD038 (8) and AZD9592 were evaluated in HT-55, Detroit 562, HT-29 and KYSE-150 xenograft models.

HT-55 (COBIOER) tumor model was established by injecting 5×10⁶ cells suspended in 0.1 mL PBS. 10 days after tumor inoculation, mice with average tumor size ˜117 mm³ were selected and assigned into 5 groups using stratified randomization (n=4 per group) based upon their tumor volumes. Mice were treated with a single (on day 0) intravenous injection of AZD9592 at 4 mg/kg, Bsab 67-LD038 (8) at 3 mg/kg, or Amivantamab at 10 mg/kg.

Detroit 562 (COBIOER) tumor model was established by injecting 3×10⁶ cells suspended in 0.1 mL PBS. 14 days after tumor inoculation, mice with average tumor size ˜130 mm³ were selected and assigned into 2 groups using stratified randomization (n=6 per group) based upon their tumor volumes. Mice were treated with a single (on day 0) intravenous injection of Bsab 67-LD038 (8) at 3.5 mg/kg.

HT-29 (ATCC) tumor model was established by injecting 2×10⁶ cells suspended in 0.1 mL PBS. 11 days after tumor inoculation, mice with average tumor size ˜127 mm³ were selected and assigned into 5 groups using stratified randomization (n=5 per group) based upon their tumor volumes. Mice were treated with a single (on day 0) intravenous injection of AZD9592 at 4 mg/kg, Bsab 67-LD038 (8) at 3 mg/kg, or Amivantamab at 10 mg/kg.

KYSE-150 (COBIOER) tumor model was established by injecting 5×10⁶ cells suspended in 0.1 mL PBS mixed with Matrigel (1:1). 12 days after tumor inoculation, mice with average tumor size ˜126 mm³ were selected and assigned into 4 groups using stratified randomization (n=3 per group) based upon their tumor volumes. Mice were treated with a single (on day 0) intravenous injection of Bsab 67-LD038 (8) at 5 mg/kg, or Amivantamab at 10 mg/kg.

The tumor size and body weight were measured as described before. Animal body weight was monitored as an indirect measure of tolerability. No mice showed significant weight loss in any of the treatment groups. There were no morbidity and deaths during the treatment duration.

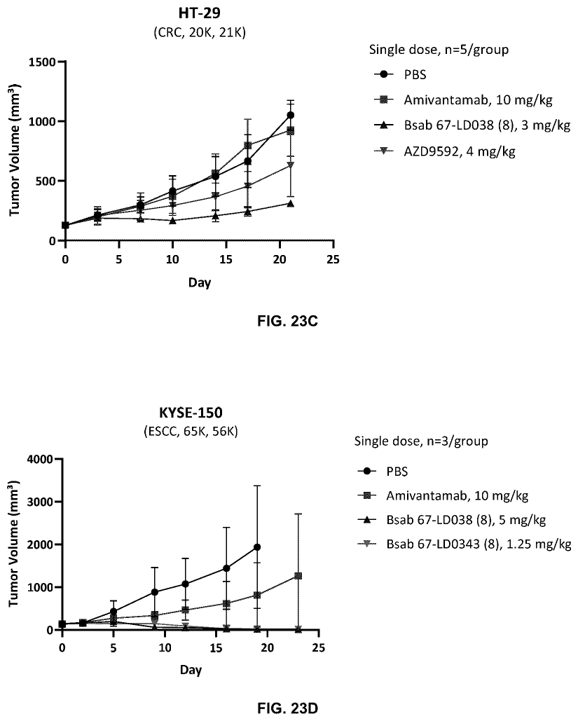

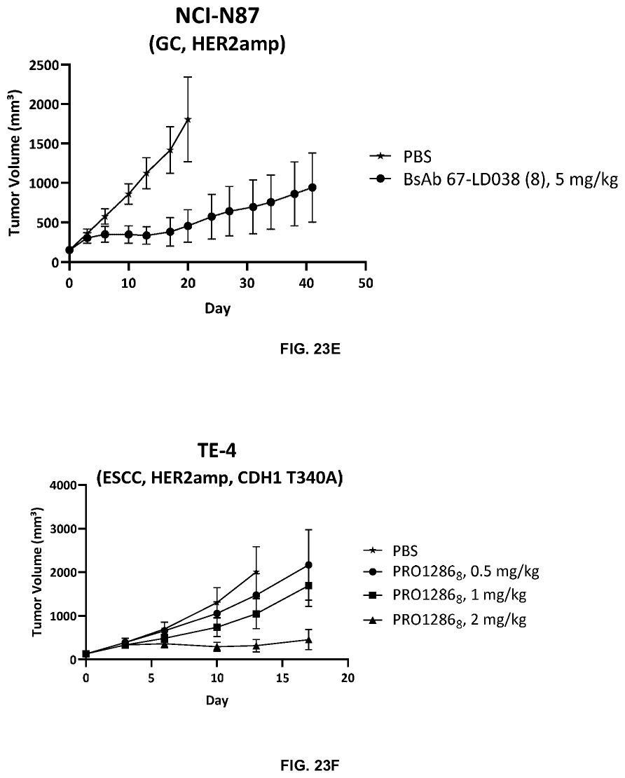

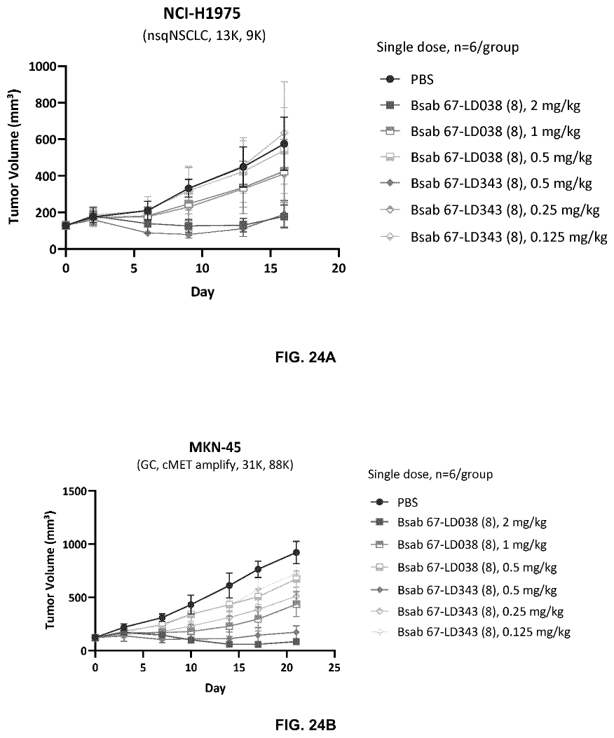

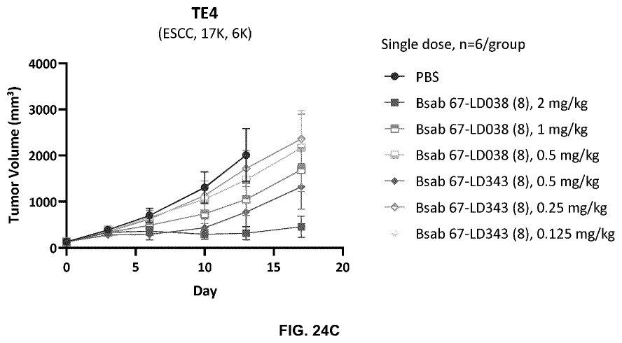

As shown in FIGS. 23A-23D, Bsab 67-LD038 (8) inhibits tumor cell lines without actionable genomic alterations in vivo.

### Example 33: In Vivo Efficacy of Bsab 67-LD038 (8)

The in vivo anti-tumor activity of Bsab 67-LD038 (8) was evaluated in NCI-N87 and TE-4 xenograft models.

NCI-N87 tumor model was established by injecting 5×10⁶ cells suspended in 0.1 mL PBS. 3 days after tumor inoculation, mice with average tumor size ˜154 mm³ were selected and assigned into 8 groups using stratified randomization (n=6 per group) based upon their tumor volumes. Mice were treated with a single (on day 0) intravenous injection of Bsab 67-LD038 (8) at 5 mg/kg.

TE-4 tumor model was established by injecting 5×10⁶ cells suspended in 0.1 mL PBS. 3 days after tumor inoculation, mice with average tumor size ˜154 mm³ were selected and assigned into 8 groups using stratified randomization (n=6 per group) based upon their tumor volumes. Mice were treated with a single (on day 0) intravenous injection of Bsab 67-LD038 (8) at 0.5 mg/kg, Bsab 67-LD038 (8) at 1 mg/kg, or Bsab 67-LD038 (8) at 2 mg/kg.

The tumor size and body weight were measured as described before. Animal body weight was monitored as an indirect measure of tolerability. No mice showed significant weight loss in any of the treatment groups. There were no morbidity and deaths during the treatment duration.

As shown in FIGS. 23E-23F, Bsab 67-LD038 (8) inhibits tumor cell lines without actionable genomic alterations in vivo.

### Example 34: In Vivo Efficacy of Bsab 67-LD038 (8) and Bsab 67-LD343 (8)

The in vivo anti-tumor activities of Bsab 67-LD038 (8) and Bsab 67-LD343 (8) were evaluated in NCI-H1975, MKN-45 and TE4 xenograft models.

NCI-H1975 (Procell) tumor model was established by injecting 5×10⁶ cells suspended in 0.1 mL PBS. 21 days after tumor inoculation, mice with average tumor size ˜128 mm³ were selected and assigned into 7 groups using stratified randomization (n=6 per group) based upon their tumor volumes. Mice were treated with a single (on day 0) intravenous injection of Bsab 67-LD038 (8) at 2, 1, 0.5 mg/kg, or Bsab 67-LD343 (8) at 0.5, 0.25, 0.125 mg/kg.

MKN-45 (Procell) tumor model was established by injecting 5×10⁶ cells suspended in 0.1 mL PBS. 6 days after tumor inoculation, mice with average tumor size ˜122 mm³ were selected and assigned into 7 groups using stratified randomization (n=6 per group) based upon their tumor volumes. Mice were treated with a single (on day 0) intravenous injection of Bsab 67-LD038 (8) at 2, 1, 0.5 mg/kg, or Bsab 67-LD343 (8) at 0.5, 0.25, 0.125 mg/kg.

TE4 (COBIOER) tumor model was established by injecting 5×10⁶ cells suspended in 0.1 mL PBS. 11 days after tumor inoculation, mice with average tumor size ˜127 mm³ were selected and assigned into 7 groups using stratified randomization (n=6 per group) based upon their tumor volumes. Mice were treated with a single (on day 0) intravenous injection of Bsab 67-LD038 (8) at 2, 1, 0.5 mg/kg, or Bsab 67-LD343 (8) at 0.5, 0.25, 0.125 mg/kg.

The tumor size and body weight were measured as described before. Animal body weight was monitored as an indirect measure of tolerability. No mice showed significant weight loss in any of the treatment groups. There were no morbidity and deaths during the treatment duration.

As shown in FIGS. 24A-24C, Bsab 67-LD038 (8) produced robust anti-tumor effect in a dose-dependent manner.

### Example 35: PK of Bsab 67-LD038 (8)

Bsab 67, Bsab 67-LD038 (8), AZD9592-Ab and AZD9592 were intravenously administered at 3 mg/kg to male Sprague Dawley rats (n=3 per group). Jugular vein blood was sampled from each rat at 10 min, 4 h, 1 d, 3 d, 7 d, 14 d and 21 d post dosing. Total antibody (TAb) concentration of Bsab 67, Bsab 67-LD038 (8), AZD9592-Ab and AZD9592 in plasma was captured by human cMET protein and detected by goat anti-human IgG Fc (HRP), calculated using GraphPad Prism 8.0 software.

Bsab 67-LD038 (8) was intravenously administered at 5 mg/kg to NCI-N87 tumor-bearing Balb/c mice (n=6 per group). Orbital blood was cross-sampled from each mouse at 10 min, 4 h, 1 d, 4 d, 7 d, 10 d, 14 d and 21 d post dosing. Total antibody (TAb) concentration of Bsab 67-LD038 (8) in plasma was captured by human cMET protein and detected by goat anti-human IgG Fc (HRP), calculated using GraphPad Prism 8.0 software.

Bsab 67, Bsab 67-LD038 (8), AZD9592-Ab and AZD9592 were intravenously administered at 3 mg/kg to NOD-SCID mice (n=3 per group). Vein blood was sampled from each rat at 10 min, 4 h, 1 d, 3 d, 7 d, 14 d and 21 d post dosing. Total antibody (TAb) concentration of Bsab 67, Bsab 67-LD038 (8), AZD9592-Ab and AZD9592 in plasma was captured by human cMET protein and detected by goat anti-human IgG Fc (HRP), calculated using GraphPad Prism 8.0 software.

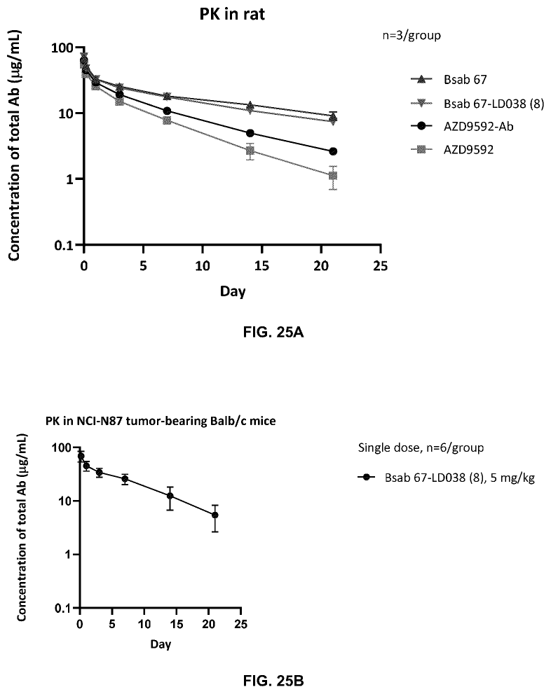

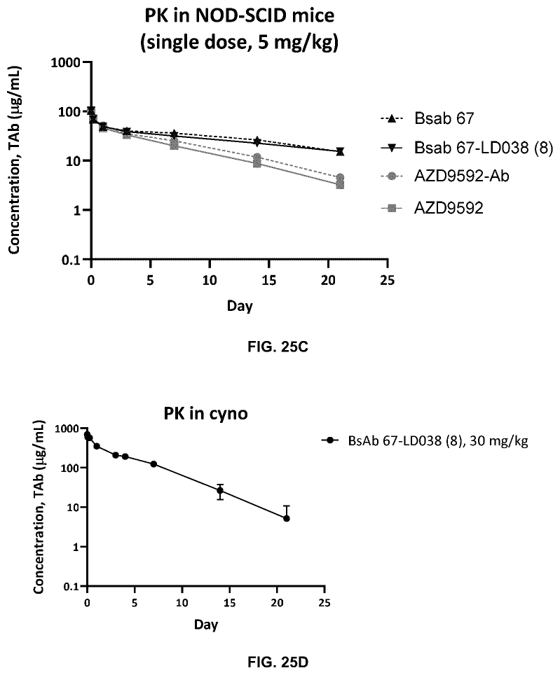

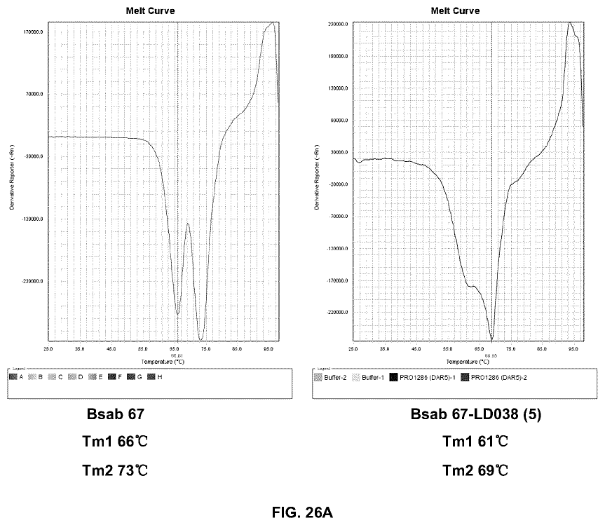

As shown in FIGS. 25A-25C, Bsab 67-LD038 (8) shows excellent PK profiles.

### Example 36: Pilot TK of Bsab 67-LD038 (8) in Cynomolgus Monkeys

Bsab 67-LD038 (8) was intravenously administered at 30 mg/kg to cynomolgus monkeys (n=2 per group). Blood was sampled from each cynomolgus monkey at 10 min, 4 h, 1 d, 4 d, 7 d, 10 d, 14 d and 21 d post dosing. Total antibody (TAb) concentration of Bsab 67-LD038 (8) in plasma was captured by human cMET protein and detected by goat anti-human IgG Fc (HRP), calculated using GraphPad Prism 8.0 software.

As shown in FIG. 25D, Bsab 67-LD038 (8) exhibits favorable toxicity and PK profiles. Cynomolgus monkeys were well tolerated. The toxicity profile was payload driven, including decreased erythropoiesis and leukocytes and principal toxicity residing in bone marrow.

### Example 37: Heat-Stability of Bsab 67-LD038(5)

Differential scanning fluorimetry (DSF) experiments were performed using Real-Time PCR Detection System (Applied Biosystems 7500). Proteins were mixed with SYPRO Orange fluorescent dye (MERCK, S5692) and diluted to 0.5 mg/mL in 20 mM histidine buffer (pH6.0). The final concentration of SYPRO Orange was 10×. Proteins were heated from 25 to 95° C., using a heating rate of 1° C./min. A fluorescence measurement was taken every 30 seconds. Melting temperatures were calculated using the instrument software.

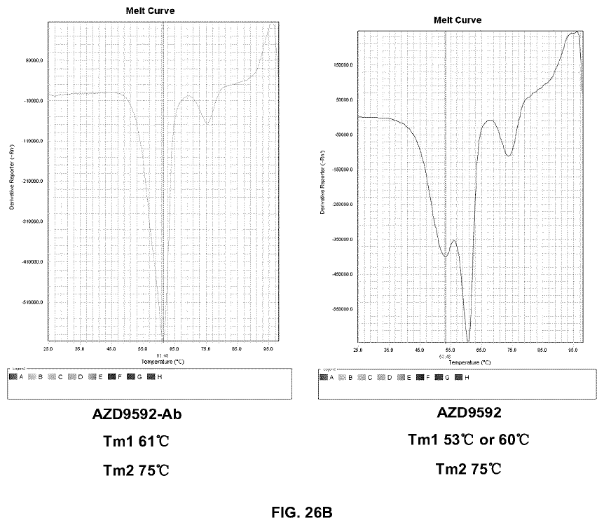

As shown in FIGS. 26A-26B, Bsab 67-LD038(5) is more heat-stable than AZD9592.

### Example 38: Hydrophilicity of Bsab 67-LD038(5)

The hydrophobicity of test articles was measured in Waters® e2695 Separations Module with UV detection at 280 nm using TSKgel Butyl-NPR column (Tosoh Bioscience, TO0042168). The mobile phase A is 50 mM phosphate buffer, 1.5 M ammonium sulfate, pH 7.0. Mobile phase B is 50 mM phosphate buffer pH 7.0, 20% isopropanol. The flow rate is 0.5 mL/min at room temperature. Use Empower™3 SOFTWARE to analyze the peak retention time.

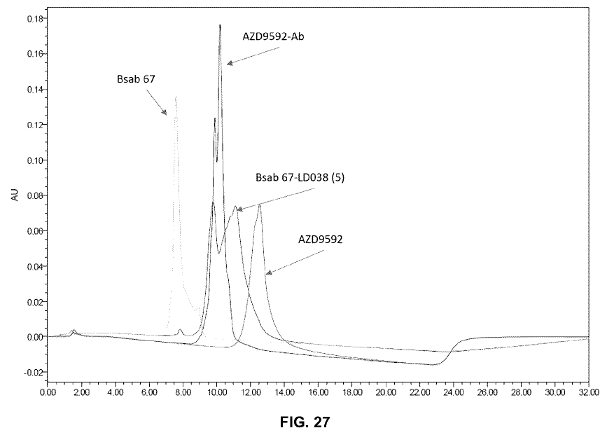

As shown in FIG. 27, Bsab 67-LD038(5) and its parent mAb are more hydrophilic than AZD9592 and its parent mAb.

### Example 39: Kinetic Binding Assay of Bsab 67 and Bsab 67-LD038 (5)

Recombinant proteins consisting of the EGFR and cMET extracellular domain (ECD) linked to His tag were purchased from ACRO Biosystems and SinoBiological. Bsab 67 or Bsab 67-LD038 (5) (13 nM) was immobilized on anti-human IgG Fc biosensors (ForteBio). For EGFR protein, binding assays using varying concentrations from 100 nM down to 1.56 nM of recombinant proteins (human, cynomolgus, rat, mouse EGFR) in solution were performed using Octet RED (ForteBio). For cMET protein, binding assays using varying concentrations from 200 nM down to 3.12 nM of recombinant proteins (human, cynomolgus, rat, mouse cMET) in solution were performed using Octet RED (ForteBio). Association time was set at 180 s and dissociation time was set at 300 s. Binding affinity was calculated by Data Acquisition 6.3 software (ForteBio) using a 1:1 binding model.

The results were as shown in Table 12. Bsab 67 and Bsab 67-LD038 (5) bind to human and monkey EGFR and cMET, but not to mouse and rat EGFR and cMET.

TABLE 12

| | Human | Cyno | Mouse | Rat | Human | Cyno | Mouse | Rat |
|---|---|---|---|---|---|---|---|---|
| | EGFR | EGFR | EGFR | EGFR | cMET | cMET | cMET | cMET |
| Antibody | (nM) | (nM) | (nM) | (nM) | (pM) | (pM) | (pM) | (pM) |
| Bsab 67 | 4.4 | 8.1 | NA | NA | 3 | 3 | NA | NA |
| Bsab 67- | 4.5 | 9 | NA | NA | 2 | 2 | NA | NA |
| LD038 (5) | | | | | | | | |

### Example 40: Binding Specificity of Bsab 67-LD038 (5) and Bsab 67

Binding specificity was tested by ELISA according to the standard protocol. Specifically, 96-well plates were coated with 100 ng/well human EGFR, human HER2, human HER3, human HER4, human cMET, human Sema3A recombinant protein in PBS, incubated overnight at 4° C. Plates were washed twice by TBS+0.05% Tween20. 200 μL blocking buffer (2% BSA in PBS) was added to each well and incubated at 37° C. for 2 h. After wash, Serial diluted antibodies were added to the ELISA plate with 100 μL per well and incubated for 1 h at room temperature. Then plates were washed for 3 times. HRP conjugated anti-human Fc antibody solution (Abcam, ab98624) was added to the plate with 100 μL per well. The plates were incubated at room temperature for 1 h and then washed for 3 times. TMB solution was added to plates with 100 μL per well, placed at room temperature for 5-15 mins, then the stop solution (2 M H₂SO₄) was added with 50 μL per well. Finally, read the absorbance at A₄₅₀ and A₆₃₀.

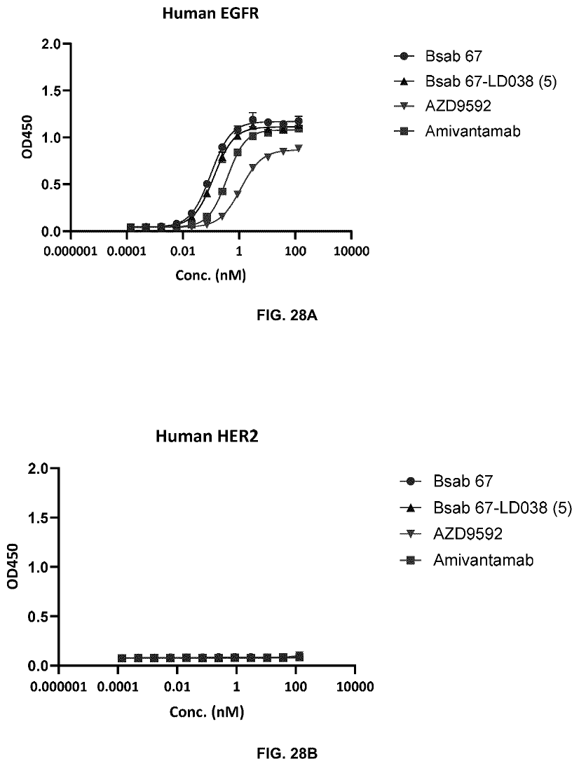

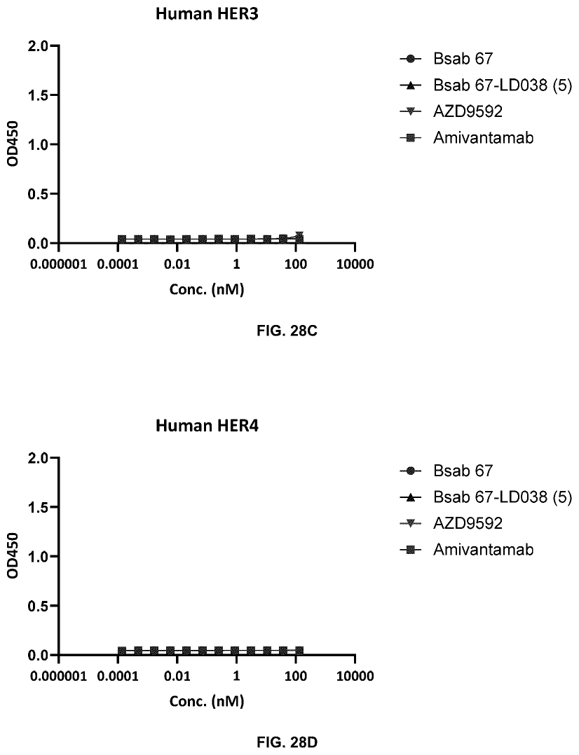

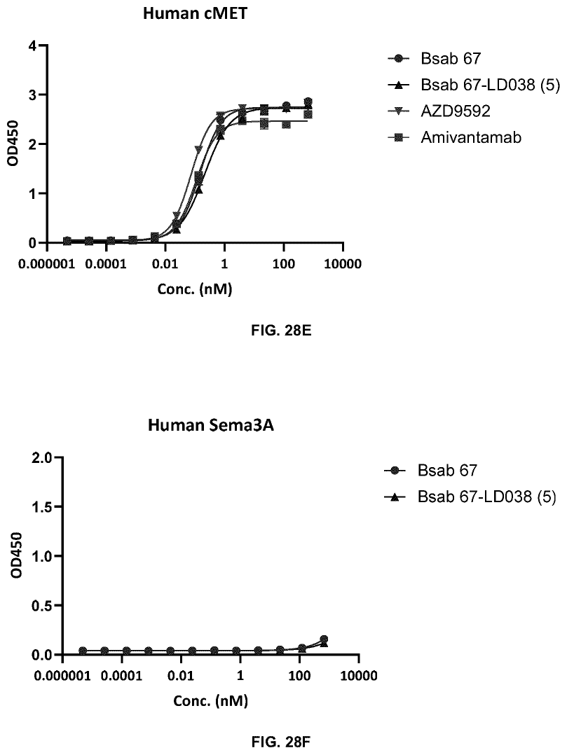

As shown in FIGS. 28A-28F, Bsab 67-LD038 (5) and Bsab 67 showed no binding to EGFR and cMET family members.

### Example 41: Binding Activity of Bsab 67-LD038 (5) and Bsab 67

EGFR and cMET co-expressed tumor cell lines including EBC-1 (JCRB JCRB0820, Provided by Shanghai EK-Bioscience), NCI-H292 (ATCC® CRL-1848, Provided by COBIOER), NCI-H1975 (ATCC® CRL-5908, Provided by Procell), CAL-27 (ATCC® CRL-2095, Provided by COBIOER), KYSE-150 (DSMZ ACC 375, Provided by COBIOER), SNU-5 (ATCC® CRL-5973, Provided by COBIOER), HT-29 (ATCC® HTB-38), ACHN (ATCC® CRL-1611, Provided by CRO), THP-1 (ATCC® TIB-202, Provided by COBIOER) were selected for evaluating antibody binding activity by flow cytometry. EBC-1, ACHN and CAL-27 were cultured with DMEM medium (Gibco, Cat #C11995500BT) containing 10% FBS. NCI-H292, NCI-H1975, KYSE-150 and THP-1 were cultured with RPMI 1640 (Gibco, Cat #11875093) containing 10% FBS. HT-29 was cultured with McCoy's 5a medium (Gibco, Cat #16600082) containing 10% FBS. SNU-5 was cultured with IMDM medium (Gibco, Cat #12440053) containing 10% FBS. Each antibody was incubated with different cell lines (1×10⁵ cells per well) for 30 mins in 0.2 mL FACS buffer (1×PBS with 0.1% BSA) at 4° C. After wash, cells were incubated with 100 μL PE-conjugated anti human Fc (Abcam, Ab98596, 1:500 dilution) for 30 mins at 4° C. After wash with PBS, cells were resuspended in FACS buffer and analyzed by flow cytometer (Beckman, CytoFLEX).

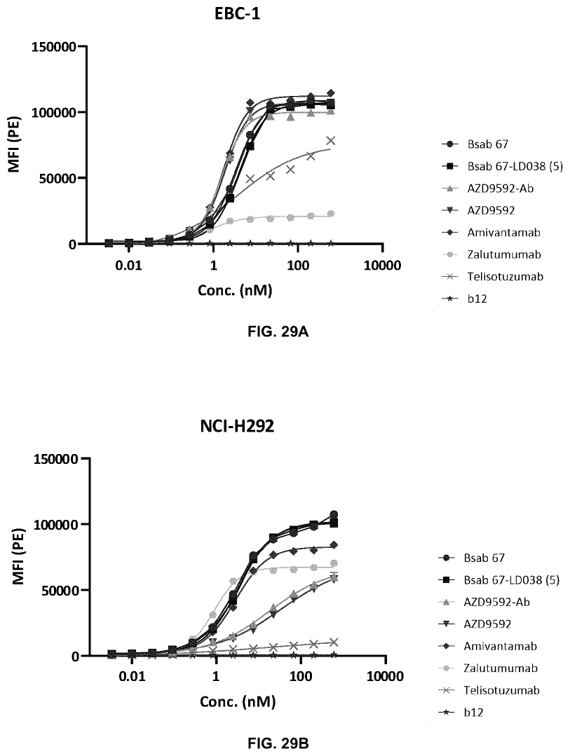

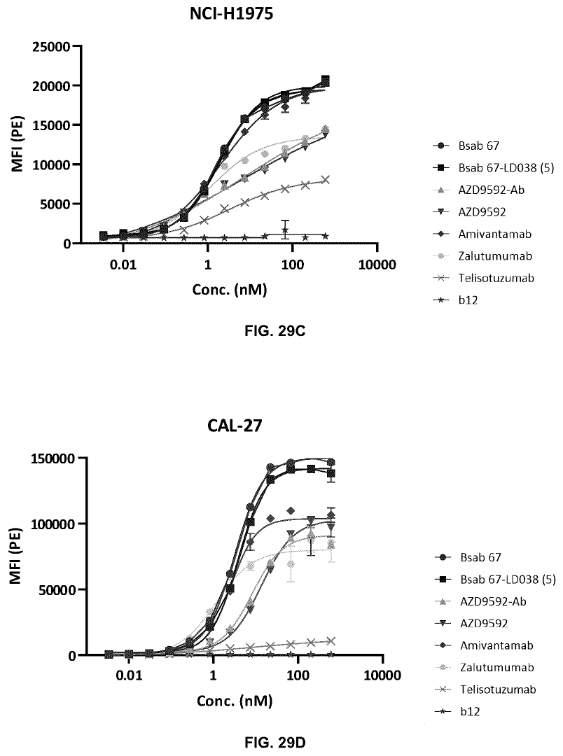

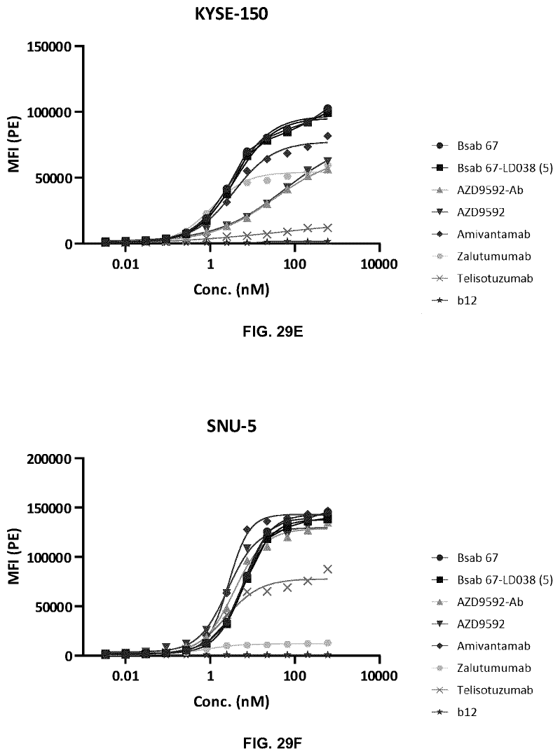

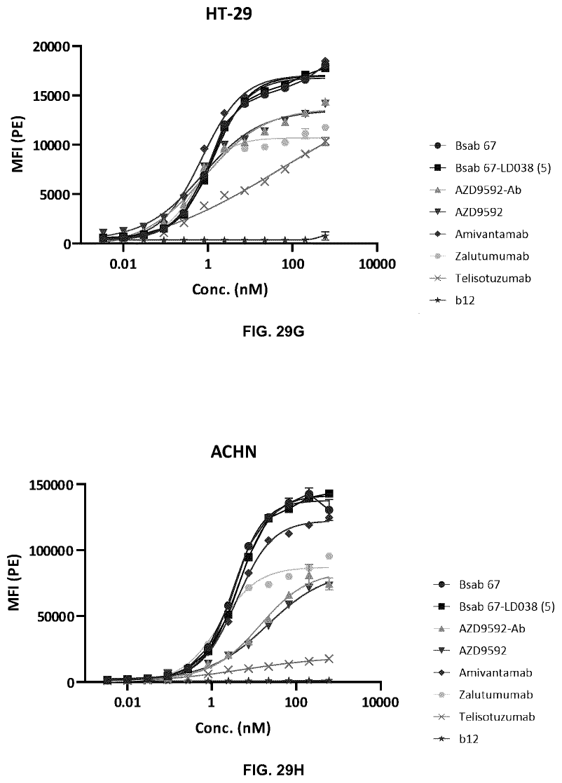

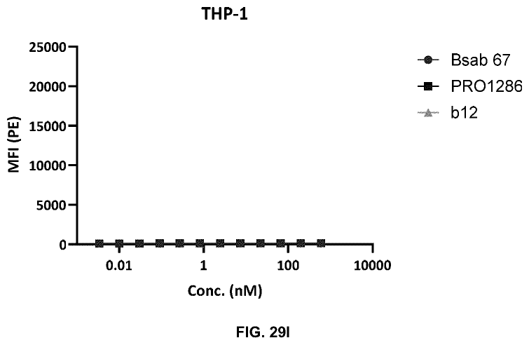

As shown in FIGS. 29A-29I, Bsab 67-LD038 (5) and Bsab 67 showed comparable binding to tumor cells.

### Example 42: Internalization of Bsab 67-LD038 (5) and Bsab 67

The internalization assay was conducted in time course. 1×10⁵ cells were incubated with 200 nM test articles (Bsab 67 or Bsab 67-LD038 (5)) for 30 mins at 4° C. in FACS buffer (1×PBS containing 0.1% BSA). Cells were washed at 4° C. to remove unbound material and kept on ice or shifted to 37° C. as needed. At progressive time points (0, 0.5, 2, 4 h), cells were stained with PE-conjugated anti-human Fc for 30 mins at 4° C. and analyzed by flow cytometry. Internalization rate was calculated by subtracting the mean fluorescence intensity (MFI) of cell surface-bound antibody at 37° C. at each timepoint from the MFI of cell surface-bound antibody at 4° C. at time 0, then divided by the MFI of cell surface-bound antibody at 4° C. at time 0.

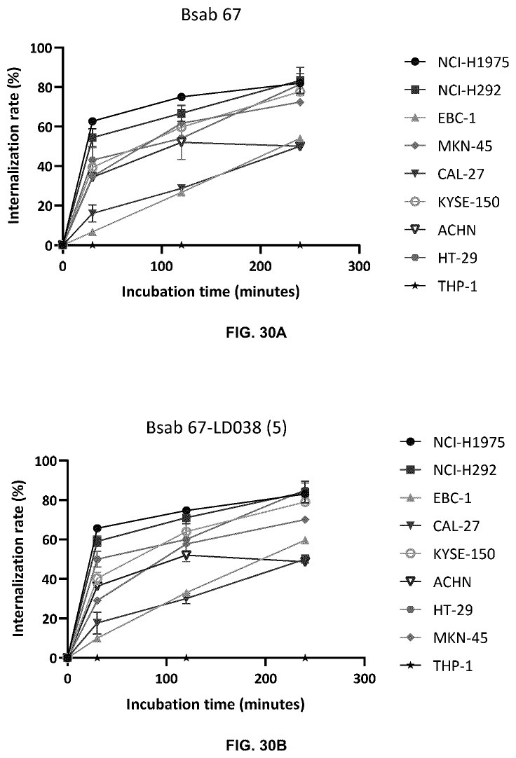

As shown in FIGS. 30A-30B, Bsab 67 and Bsab 67-LD038 (5) show robust internalization in various tumor cell lines.

### Example 43: Cytotoxicity of Bsab 67-LD038 (5) and Bsab 67

Cells were harvested and seeded into 96-well plates (Coring, 3917) for overnight culture. In the next day, serial diluted test articles (Bsab 67-LD038 (5), AZD9592, Exatecan or b12-LD038 (5)) were added to cells and the incubation was lasting for 144 h at 37° C. After that, 40 μL CellTiter-Glo (Promega) was added to each well and incubated for 5 mins at room temperature. Luciferase readings were collected by microplate reader. All readings were normalized as percentage of viable cells in the untreated control wells and the IC₅₀ values were calculated by Prism software.

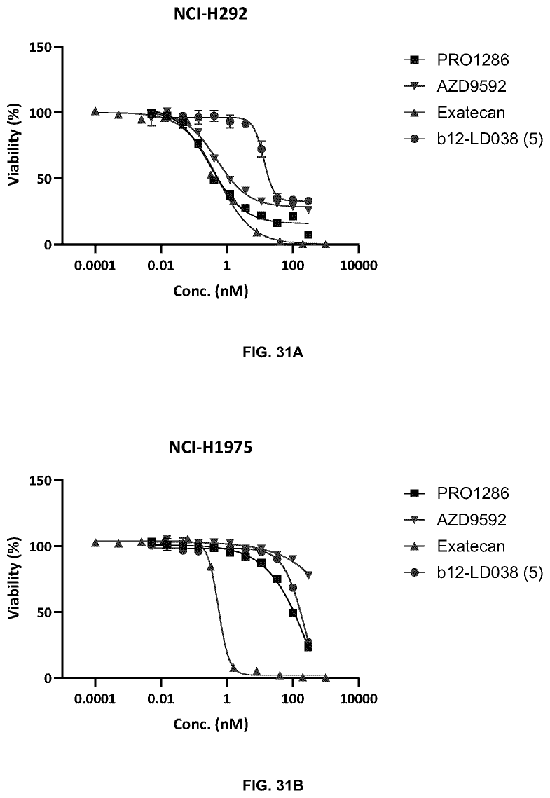

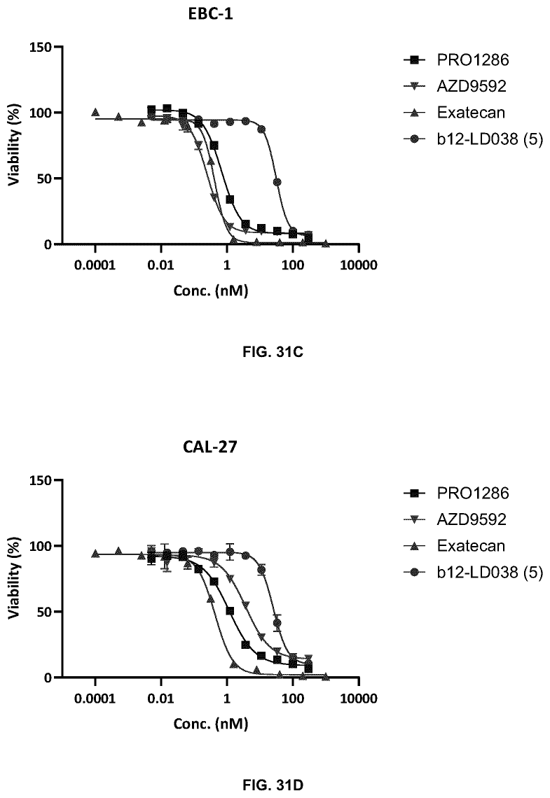

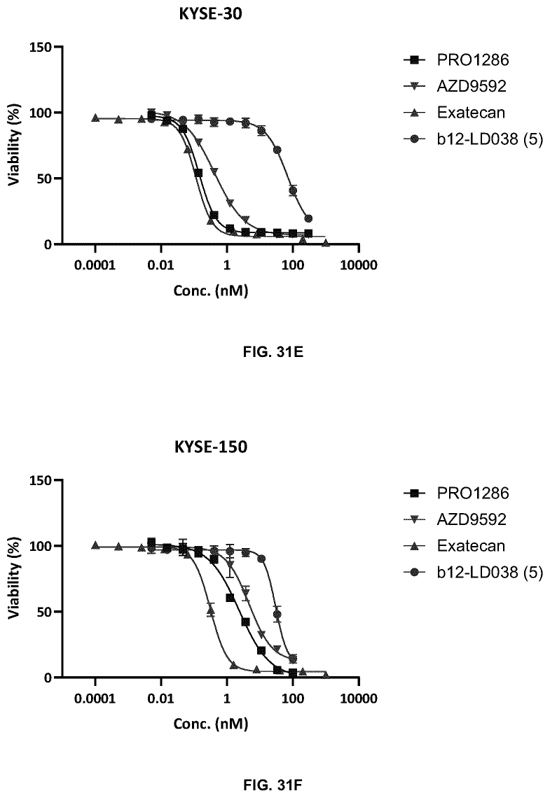

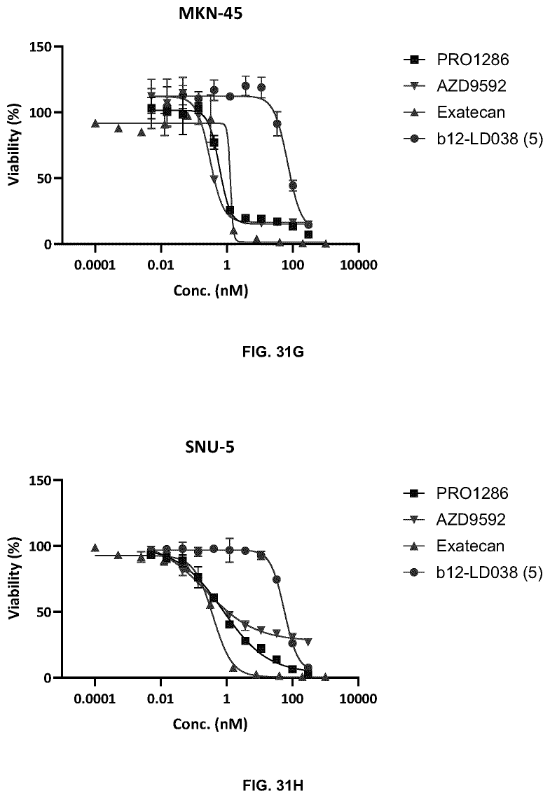

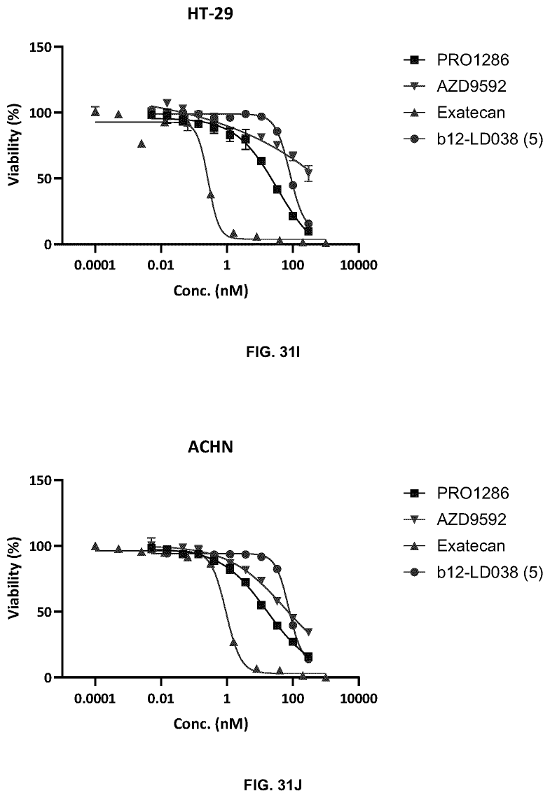

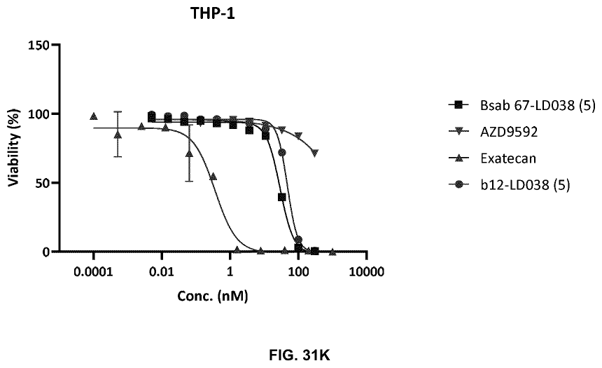

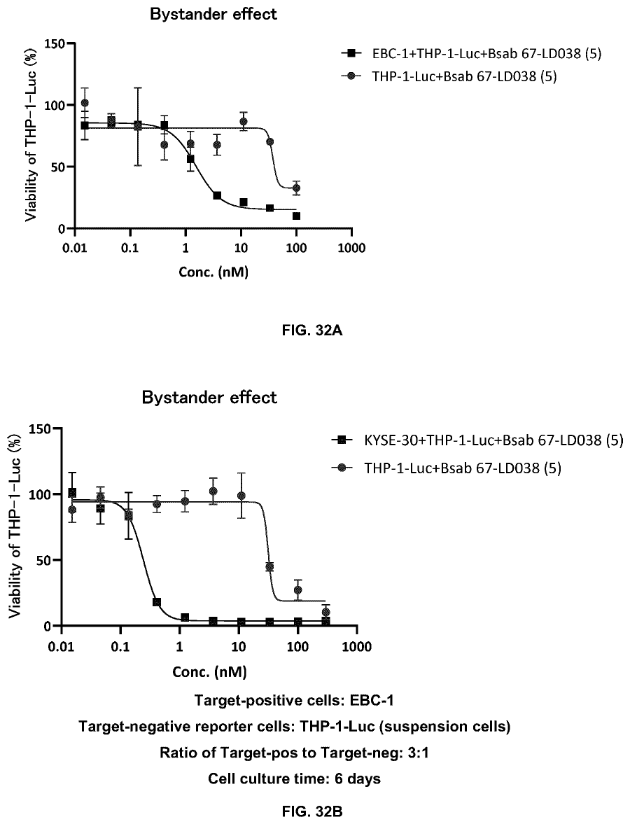

As shown in FIGS. 31A-31K, Bsab 67-LD038 (5) show superior cytotoxicity than AZD9592 in various tumor cell lines.

### Example 44: Bystander Effect of Bsab 67-LD038 (5)

Target positive cells EBC-1 or KYSE-30 and EGFR & cMET negative cells THP-1-Luc were inoculated into 96-well plates (Nunc) with prespecified densities (EBC-1:3000 cells/well; KYSE-30:1000 cells/well; THP-1-Luc:1000 cells/well). In the next day, Bsab 67-LD038 (5) was added and incubated with cells for 144 h at 37° C. Viable cells were detected by adding 80 μL/well Bio-Lite® reagent (Promega) and luminescence intensity was read by plate reader (Molecular Devices SpectraMax M5). Data was plotted as raw luminescence units (RLU) against the logarithm of Bsab 67-LD038 (5) molar concentration. IC₅₀ values were determined by GraphPad Prism software.

As shown in FIGS. 32A-32B, Bsab 67-LD038 (5) show great bystander effect in various tumor cell lines.

### Example 45: In Vivo Efficacy of Bsab 67-LD038 (5)

The in vivo anti-tumor activities of Amivantamab, Bsab 67-LD038 (5), Bsab 67-LD038 (8) and AZD9592 were evaluated in following xenograft models.

EBC-1 (EK-Bioscience) tumor model was established by injecting 3×10⁶ cells suspended in 0.1 mL PBS mixed with Matrigel (1:1). 10 days after tumor inoculation, mice with average tumor size ˜125 mm³ were selected and assigned into 5 groups using stratified randomization (n=6 per group) based upon their tumor volumes. Mice were treated with a single (on day 0) intravenous injection of Bsab 67-LD038 (5) at 4.8, 1.6 mg/kg, or AZD9592 at 4, 1.3 mg/kg.

CAL-27 (COBIOER) tumor model was established by injecting 5×10⁶ cells suspended in 0.1 mL PBS mixed with Matrigel (1:1). 28 days after tumor inoculation, mice with average tumor size ˜120 mm³ were selected and assigned into 4 groups using stratified randomization (n=6 per group) based upon their tumor volumes. Mice were treated with a single (on day 0) intravenous injection of Bsab 67-LD038 (5) at 4.8 mg/kg, AZD9592 at 4 mg/kg, or Amivantamab at 10 mg/kg.

NCI-H1975 (Procell) tumor model was established by injecting 5×10⁶ cells suspended in 0.1 mL PBS mixed with Matrigel (1:1). 25 days after tumor inoculation, mice with average tumor size ˜160 mm³ were selected and assigned into 6 groups using stratified randomization (n=6 per group) based upon their tumor volumes. Mice were treated with a single (on day 0) intravenous injection of Bsab 67-LD038 (5) at 4.8, or 1.6 mg/kg, Bsab 67-LD038 (8) at 3, or 1 mg/kg, AZD9592 at 4, 1.3 mg/kg.

MKN-45 (Procell) tumor model was established by injecting 5×10⁶ cells suspended in 0.1 mL PBS mixed with Matrigel (1:1). 7 days after tumor inoculation, mice with average tumor size ˜120 mm³ were selected and assigned into 6 groups using stratified randomization (n=6 per group) based upon their tumor volumes. Mice were treated with a single (on day 0) intravenous injection of Bsab 67-LD038 (5) at 4.8, or 2.4 mg/kg, AZD9592 at 4, 2 mg/kg.

The tumor size and body weight were measured as described before. Animal body weight was monitored as an indirect measure of tolerability. No mice showed significant weight loss in any of the treatment groups. There were no morbidity and deaths during the treatment duration.

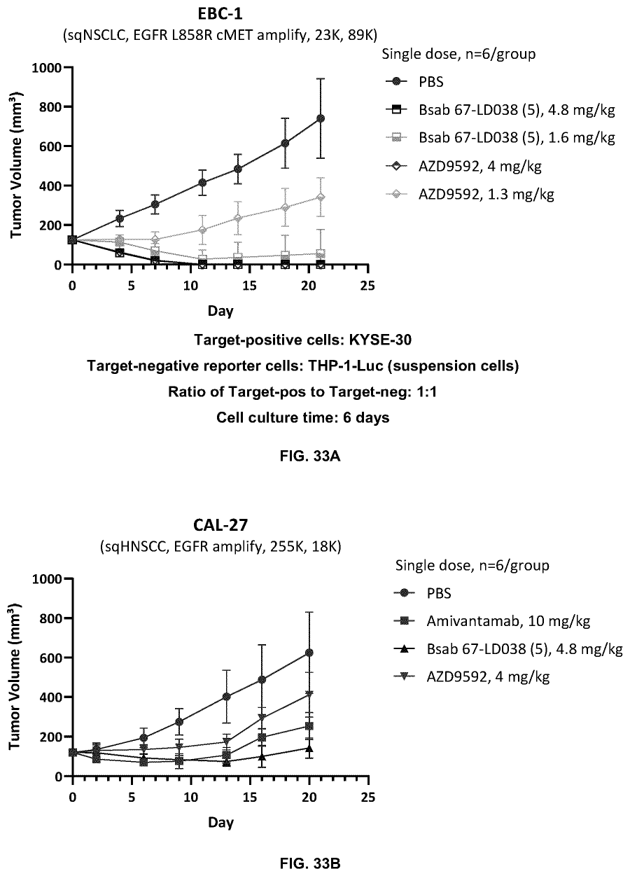

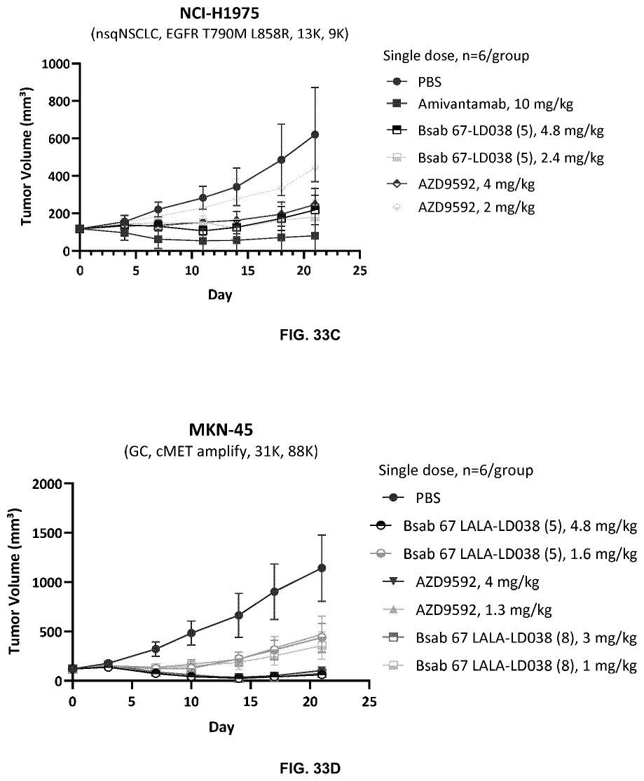

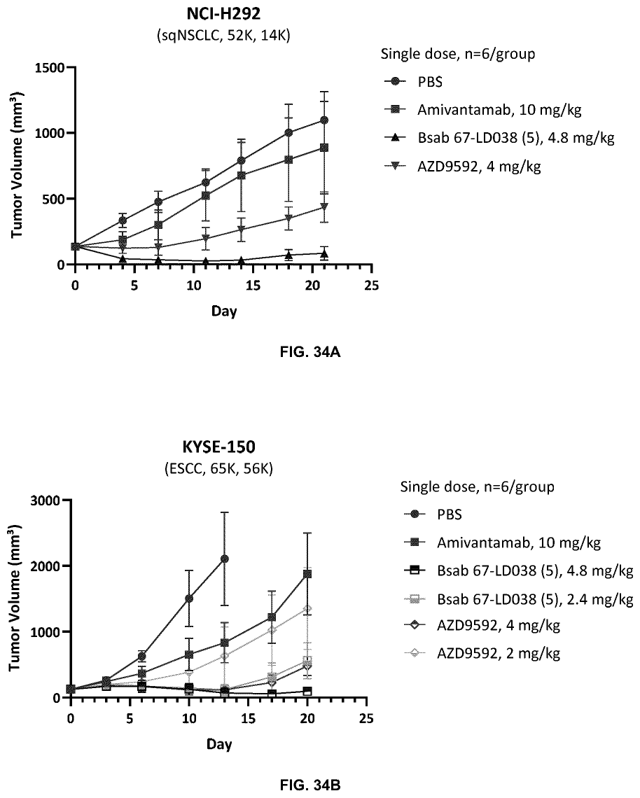

As shown in FIGS. 33A-33D, Bsab 67-LD038 (5) inhibits tumor cell lines with actionable genomic alterations in vivo.

### Example 46: In Vivo Efficacy of Bsab 67-LD038 (5)

The in vivo anti-tumor activities of Amivantamab, Bsab 67-LD038 (5) and AZD9592 were evaluated in NCI-H292, KYSE-150, HT-29 and ACHN xenograft models.

NCI-H292 (COBIOER) tumor model was established by injecting 5×10⁶ cells suspended in 0.1 mL PBS mixed with Matrigel (1:1). 7 days after tumor inoculation, mice with average tumor size ˜136 mm³ were selected and assigned into 4 groups using stratified randomization (n=6 per group) based upon their tumor volumes. Mice were treated with a single (on day 0) intravenous injection of Bsab 67-LD038 (5) at 4.8 mg/kg, AZD9592 at 4 mg/kg, or Amivantamab at 10 mg/kg.

KYSE-150 (COBIOER) tumor model was established by injecting 5×10⁶ cells suspended in 0.1 mL PBS. 12 days after tumor inoculation, mice with average tumor size ˜126 mm³ were selected and assigned into 6 groups using stratified randomization (n=6 per group) based upon their tumor volumes. Mice were treated with a single (on day 0) intravenous injection of Bsab 67 analogue-LD038 (5) at 4.8, 2.4 mg/kg, AZD9592 at 4, 2 mg/kg or Amivantamab at 10 mg/kg.

HT-29 (ATCC) tumor model was established by injecting 2×10⁶ cells suspended in 0.1 mL PBS. 14 days after tumor inoculation, mice with average tumor size ˜126 mm³ were selected and assigned into 4 groups using stratified randomization (n=6 per group) based upon their tumor volumes. Mice were treated with a single (on day 0) intravenous injection of Bsab 67 (5) at 4.8 mg/kg, AZD9592 at 4 mg/kg, or Amivantamab at 10 mg/kg.

ACHN (innoModels) tumor model was established by injecting 5×10⁶ cells suspended in 0.1 mL PBS mixed with Matrigel (1:1). 6 days after tumor inoculation, mice with average tumor size ˜160 mm³ were selected and assigned into 4 groups using stratified randomization (n=6 per group) based upon their tumor volumes. Mice were treated with a single (on day 0) intravenous injection of Bsab 67-LD038 (5) at 4.8 mg/kg, AZD9592 at 4 mg/kg, or Amivantamab at 10 mg/kg.

The tumor size and body weight were measured as described before. Animal body weight was monitored as an indirect measure of tolerability. No mice showed significant weight loss in any of the treatment groups. There were no morbidity and deaths during the treatment duration.

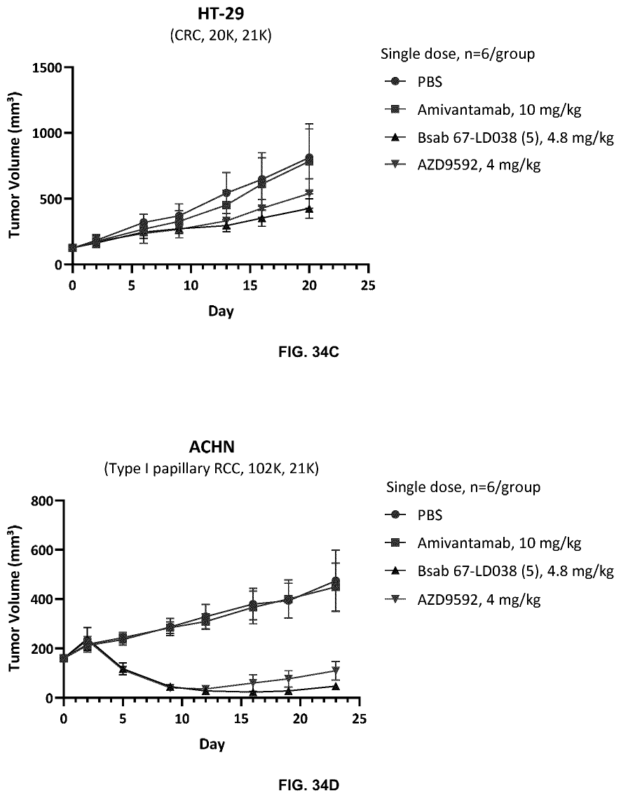

As shown in FIGS. 34A-34D, Bsab 67-LD038 (5) inhibits tumor cell lines without actionable genomic alterations in vivo.

### Example 47: PK of Bsab 67-LD038 (5)

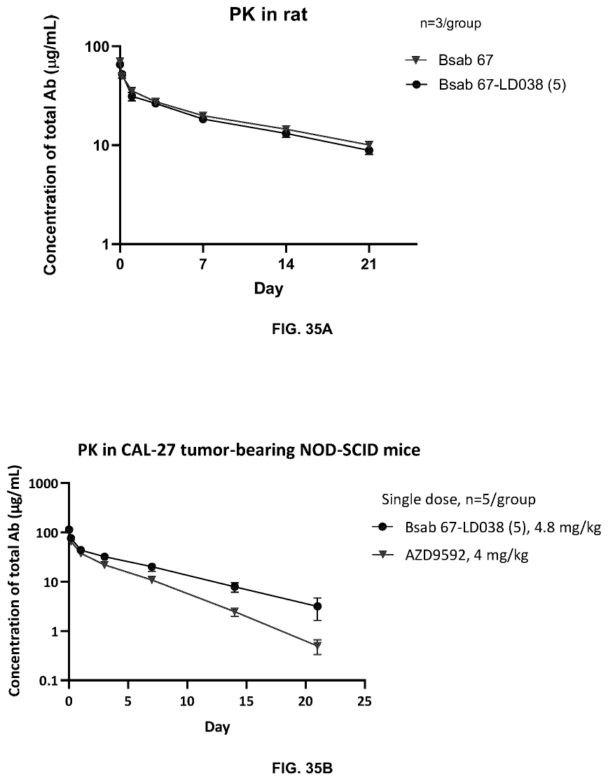

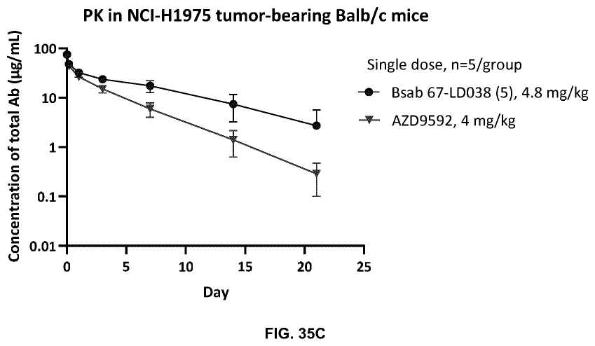

Bsab 67 and Bsab 67-LD038 (5) were intravenously administered at 3 mg/kg to male Sprague Dawley rats (n=3 per group). Jugular vein blood was sampled from each rat at 10 min, 4 h, 1d, 3d, 7d, 14d and 21d post dosing. Total antibody (TAb) concentration of Bsab 67 and Bsab 67-LD038 (5) in plasma was captured by human cMET protein and detected by goat anti-human IgG Fc (HRP), calculated using GraphPad Prism 8.0 software. As shown in FIG. 35A, Bsab 67-LD038 (5) showed excellent PK in rat.

Bsab 67-LD038 (5) at 4.8 mg/kg or AZD9592 at 4 mg/kg were intravenously administered to CAL-27 tumor-bearing Balb/c mice (n=5 per group). Orbital blood was cross-sampled from each mouse at 10 min, 4 h, 1 d, 4 d, 7 d, 10 d, 14 d and 21 d post dosing. Total antibody (TAb) concentration of Bsab 67-LD038 (8) in plasma was captured by human cMET protein and detected by goat anti-human IgG Fc (HRP), calculated using GraphPad Prism 8.0 software. As shown in FIG. 35B, Bsab 67-LD038 (5) showed excellent therapeutic window in preclinical model.

Bsab 67-LD038 (5) at 4.8 mg/kg or AZD9592 at 4 mg/kg were intravenously administered to NCI-H1975 tumor-bearing Balb/c mice (n=5 per group). Orbital blood was cross-sampled from each mouse at 10 min, 4 h, 1 d, 4 d, 7 d, 10 d, 14 d and 21 d post dosing. Total antibody (TAb) concentration of Bsab 67-LD038 (8) in plasma was captured by human cMET protein and detected by goat anti-human IgG Fc (HRP), calculated using GraphPad Prism 8.0 software. As shown in FIG. 35C, Bsab 67-LD038 (5) showed excellent therapeutic window in preclinical model.

### Example 48: Plasma Stability of Bsab 67-LD038 (5)

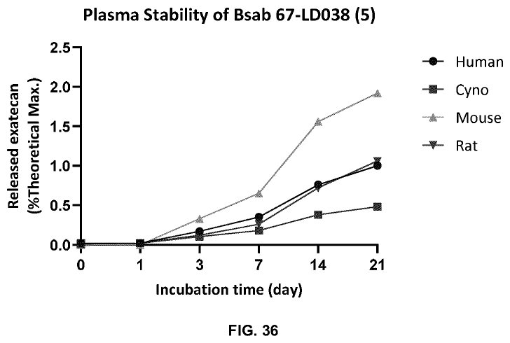

A test article was prepared to a final work solution with 0.15 mg/mL in human, monkey, rat and mouse plasma, respectively. 1 mL sample was removed into 1.5 mL tube, well packed (to avoid volume and concentration changes caused by evaporation of solution) and incubated at 37° C. for successive timepoints D0, D1, D3, D7, D14, D21, respectively. At the end of incubations, samples were collected and stored at −80° C. Free drug was analyzed by LC-MS. As shown in FIG. 36, Bsab 67-LD038 (5) conferred excellent stability in plasma.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof.

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof to streamline the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

1. A bispecific binding agent comprising:
a first binding domain that binds to EGFR; and
a second binding domain that binds to c-MET,
wherein the first binding domain comprises a heavy chain and a light chain, the heavy chain comprising a heavy chain variable (VH) region and the light chain comprising a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, wherein
the HCDR1 of the first binding domain has an amino acid sequence of SEQ ID NO: 139 or 174,
the HCDR2 of the first binding domain has an amino acid sequence of SEQ ID NO: 140 or 175,
the HCDR3 of the first binding domain has an amino acid sequence of SEQ ID NO: 141 or 176,
the LCDR1 of the first binding domain has an amino acid sequence of SEQ ID NO: 142 or 177,
the LCDR2 of the first binding domain has an amino acid sequence of DAS or KVS, and
the LCDR3 of the first binding domain has an amino acid sequence of SEQ ID NO: 143 or 178.

2. The bispecific binding agent of claim 1, wherein the second binding domain comprises a heavy chain and a light chain, the heavy chain comprising a heavy chain variable (VH) region and the light chain comprising a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, wherein the HCDR1 of the second binding domain has an amino acid sequence of SEQ ID NO: 149, 164, 194, or 235,
the HCDR2 of the second binding domain has an amino acid sequence of SEQ ID NO: 150, 165, or 236,
the HCDR3 of the second binding domain has an amino acid sequence of SEQ ID NO: 151,166, or 237,
the LCDR1 of the second binding domain has an amino acid sequence of SEQ ID NO: 152, 167, or 238,
the LCDR2 of the second binding domain has an amino acid sequence of RAS, WAS, or AAS, and
the LCDR3 of the second binding domain has an amino acid sequence of SEQ ID NO: 153, 168, or 239.

3. The binding agent of claim 1, wherein the VH and VL regions of the first binding domain that binds to EGFR have amino acid sequences that are selected from the pairs of amino acid sequences set forth in the group consisting of:
(i) SEQ ID NO: 137 and SEQ ID NO: 138, respectively;
(ii) SEQ ID NO: 157 and SEQ ID NO: 158, respectively;
(iii) SEQ ID NO: 172 and SEQ ID NO: 173, respectively;
(iv) SEQ ID NO: 187 and SEQ ID NO: 188, respectively;
(v) SEQ ID NO: 198 and SEQ ID NO: 199, respectively;
(vi) SEQ ID NO: 208 and SEQ ID NO: 209, respectively;
(vii) SEQ ID NO: 218 and SEQ ID NO: 219, respectively;
(viii) SEQ ID NO: 228 and SEQ ID NO: 229, respectively;
(ix) SEQ ID NO: 243 and SEQ ID NO: 244, respectively;
(x) SEQ ID NO: 251 and SEQ ID NO: 252, respectively; and
(xi) SEQ ID NO: 259 and SEQ ID NO: 260, respectively.

4. The binding agent of claim 1, wherein the VH and VL regions of the second binding domain that binds to c-MET have amino acid sequences that are selected from the pairs of amino acid sequences set forth in the group consisting of:
(i) SEQ ID NO: 147 and SEQ ID NO: 148, respectively;
(i) SEQ ID NO: 162 and SEQ ID NO: 163, respectively;
(iii) SEQ ID NO: 182 and SEQ ID NO: 183, respectively;
(iv) SEQ ID NO: 192 and SEQ ID NO: 193, respectively;
(v) SEQ ID NO: 203 and SEQ ID NO: 204, respectively;
(vi) SEQ ID NO: 213 and SEQ ID NO: 214, respectively;
(vii) SEQ ID NO: 223 and SEQ ID NO: 224, respectively;
(viii) SEQ ID NO: 233 and SEQ ID NO: 234, respectively;
(ix) SEQ ID NO: 248 and SEQ ID NO: 249, respectively;
(x) SEQ ID NO: 254 and SEQ ID NO: 255, respectively; and
(xi) SEQ ID NO: 264 and SEQ ID NO: 265, respectively.

5-7. (canceled)

8. The binding agent of claim 1, wherein the binding agent is an antibody or an antigen-binding portion thereof.

9. (canceled)

10. The binding agent of claim 1, wherein the heavy chain variable region further comprises a heavy chain constant (CH) region.

11-13. (canceled)

14. The binding agent of claim 10, wherein the heavy chain constant region is an IgG1 constant region having an amino acid sequence set forth in SEQ ID NO: 266, SEQ ID NO: 267, SEQ ID NO: 268, SEQ ID NO: 269, or SEQ ID NO: 270.

15. The binding agent of claim 1, wherein the light chain variable region further comprises a light chain constant region.

16. (canceled)

17. The binding agent of claim 15, wherein the light chain constant region has the amino acid sequence set forth in SEQ ID NO: 271.

18. The binding agent of claim 10, wherein the heavy chain constant region comprises at least amino acid modification that decreases binding affinity to human FcγRIII.

19. A pharmaceutical composition comprising the binding agent of claim 1 and a pharmaceutically acceptable carrier.

20. A nucleic acid encoding the binding agent of claim 1.

21. A vector comprising the nucleic acid of claim 20.

22. A cell line comprising the nucleic acid of claim 20.

23. A conjugate comprising:
the bispecific binding agent of claim 1,
at least one linker attached to the bispecific binding agent;
at least one drug unit, wherein each drug unit is attached to a linker, wherein the linker optionally comprises at least one polar group.

24. The conjugate of claim 23, wherein the linker is derived from a linker compound, or a stereoisomer or salt thereof, and the linker compound comprises:
the linker unit;
a stretcher group connected to the linker unit,
an optional amino acid unit; and
the at least one polar group; wherein:
the stretcher group has an attachment site to the bispecific binding agent and an attachment site to the amino acid unit (when present) or the linker unit;
the amino acid unit (when present) has an attachment site to the stretcher group and an attachment site to the linker unit;
the linker unit has an attachment site to the amino acid unit (when present) or to the stretcher group and an attachment site to the at least one drug unit; and
the at least one polar group is attached to at least one of the linker unit, the amino acid unit, or the stretcher group.

25. The conjugate of claim 24, wherein:
(I) the linker compound comprises:
(a) the linker unit having from 1 to 4 attachment sites for the drug unit;
(b) the amino acid unit having from 1 to 12 amino acid subunits; and
(c) the at least one polar group attached to the amino acid unit, wherein the polar group comprises a polymer unit, optionally a sugar unit, and optionally a carboxyl unit, wherein the polymer unit comprises the formula: 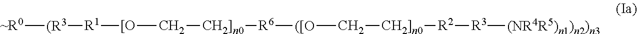
or a stereoisomer or salt thereof, wherein:
R⁰ is a functional group for attachment to a subunit of the amino acid unit;
each R¹ and R² are independently a bond or C₁-C₆ alkylene;
each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈—, triazolyl, —C₁-C₁₂ alkylene-triazolyl-, —N(polyhydroxyl group)-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R⁶ is selected from: 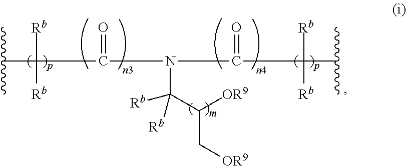
wherein:
each n3 and n4 are independently 0-1,
each R^{b} is independently H or C₁₋₆ alkyl,
each R⁹ is independently H, acetyl, —P(═O)(OH)₂, or —(CH₂)ᵥ—O—S(═O)2(OH),
each p is independently 0-6,
m is 1-4,
each v is independently 1-6, and
n2 is 1; 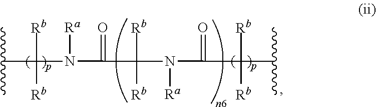
wherein:
each R^{a} is independently H or C₁₋₆ alkyl,
each R^{b} is independently H or C₁₋₆ alkyl,
n6 is 1-10,
each p is independently 0-6, and
n2 is 1; 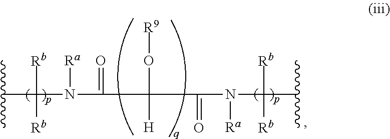
wherein:
each R^{a} is independently H or C₁₋₆ alkyl,
each R^{b} is independently H or C₁₋₆ alkyl,
each R⁹ is independently H, acetyl, —P(═O)(OH)₂, or —(CH₂)ᵥ—O—S(═O)₂(OH),
each p is independently 0-6,
q is 1-8,
each v is independently 1-6, and
n2 is 1; 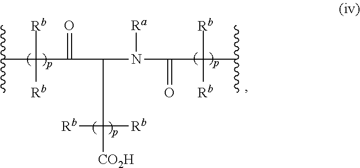
wherein:
each R^{a} is independently H or C₁₋₆ alkyl,
each R^{b} is independently H or C₁₋₆ alkyl,
each p is independently 0-6, and
n2 is 1;
—R¹⁰—[O—CH₂—CH₂]₁₋₈—R¹⁰—, wherein: (v)
each R¹⁰ is independently 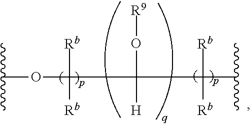
each R^{b} is independently H or C₁₋₆ alkyl,
each p is independently 1-6,
each R⁹ is independently H, acetyl, —P(═O)(OH)₂, or —(CH₂)ᵥ—O—S(═O)₂(OH), and
q is 1-8;
n2 is 1; and
—N—(R¹—X—R²—)₂, wherein: (vi)
each X is independently —NR^{a}—C(O)— or —C(O)NR^{a}—, and
n2 is 2; and
the wavy line (˜) indicates an attachment site of the amino acid unit to R⁰;
each n0 is independently 2-26;
each n1 is independently 1-6; and
n3 is 1-6;
(II) the linker compound comprises:—
(a) the linker unit having from 1 to 4 attachment sites for the drug unit;
(b) the amino acid unit having from 1 to 12 amino acid subunits; and
(c) the at least one polar group attached to the amino acid unit, wherein the polar group comprises a polymer unit, optionally a sugar unit, and optionally a carboxyl unit, wherein said polymer unit comprises the formula: 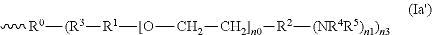
or a stereoisomer or salt thereof, wherein:
R⁰ is a functional group for attachment to a subunit of the amino acid unit;
each R¹ and R² are independently a bond or C₁-C₆ alkylene;
each R³ is independently —N(polyhydroxyl group)-, triazolyl, —C₁-C₁₂ alkylene-triazolyl-, 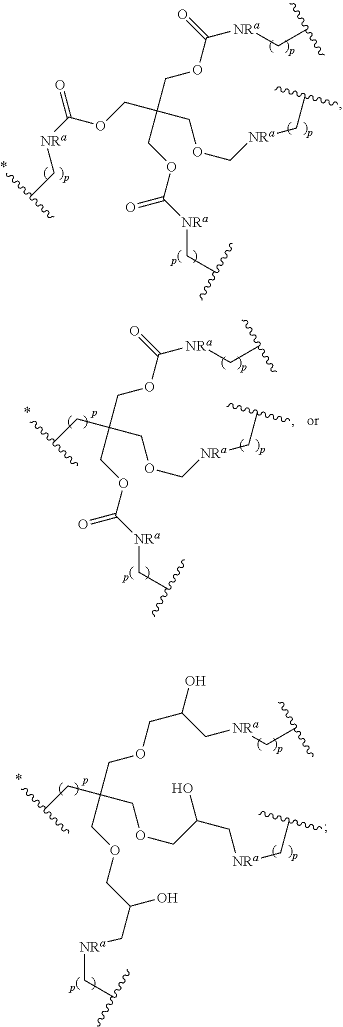
each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl; 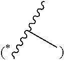
indicates an attachment site of R³ to R⁰
the wavy line 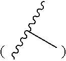
indicates an attachment site of the R³ to R¹;
each p is 1-6;
each n0 is independently 2-8;
each n1 is independently 1-6; and
n3 is 1-6;
(III) the linker compound comprises:
(a) the linker unit having from 1 to 4 attachment sites for the drug unit;
(b) the amino acid unit having from 1 to 12 amino acid subunits; and
(c) the at least one polar group attached to the amino acid unit, wherein the polar group comprises a polymer unit, optionally a sugar unit, and optionally a carboxyl unit, wherein said polymer unit comprises the formula: 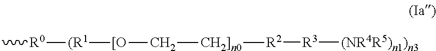
or a stereoisomer or salt thereof, wherein:
(i) R⁰ is a functional group for attachment to a subunit of the amino acid unit;
each R¹ and R² are independently a bond or C₁-C₆ alkylene;
R³ is —C(O)—;
R⁴ is H;
R⁵ is independently a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate;
the wavy line (˜) indicates an attachment site of the amino acid unit to R⁰;
n0 is independently 2-26;
n1 is 1-6; and
n3 is 1-6;
(ii) R⁰ is —C(O)—;
R¹, R², and R³ are each a bond;
R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates an attachment site of the amino acid unit to R⁰;
n0 is 6;
n1 is 1-6; and
n3 is 1;
(iii) R⁰ is a functional group for attachment to a subunit of the amino acid unit;
R¹ and R² are each, independently, a bond or C₁-C₆ alkylene;
R³ is —NR^{a}—C(O)—C₁-C₁₂ alkylene-C(O)—, wherein the alkylene is substituted with —SO₃H:
R^{a} is H or C₁₋₆ alkyl;
R⁴ and R⁵ are each independently H, a carboxyl-containing moiety, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates an attachment site of the amino acid unit to R⁰;
each n0 is independently 1-26;
n1 is 1-6; and
n3 is 1-6; or
(iv) R⁰ is 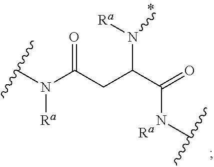
each R¹ is independently a bond or C₁-C₆ alkylene;
R² and R³ are each a bond;
R⁴ and R⁵ are each independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;
the wavy line 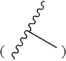
indicates an attachment site of R⁰ to the remainder of the polymer unit;
the wavy line (˜*) indicates an attachment site of the amino acid unit to R⁰;
n0 is 1-8;
n1 is 1-6; and
n3 is 2;
(IV) the linker compound comprises:
(a) the linker unit having from 1 to 4 attachment sites for the drug unit, said linker unit comprising a moiety of formula: 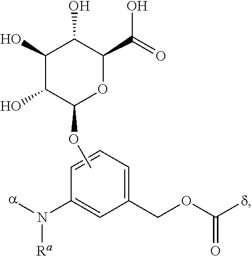
or a stereoisomer or salt thereof, wherein:
α—represents a direct or indirect attachment site to the amino acid unit;
δ—represents an attachment site to the drug unit or for a linking group attached to the drug unit; and
R^{a} is H or C₁₋₆ alkyl;
(b) the amino acid unit having from 1 to 12 amino acid subunits; and
(c) the at least one polar group attached to the amino acid unit, wherein the polar group comprises a polymer unit, optionally a sugar unit, and optionally a carboxyl unit;
(V) the linker compound comprises:
(a) the linker unit having from 1 to 4 attachment sites for the drug unit;
(b) the amino acid unit having from 1 to 12 amino acid subunits; and
(c) the at least one polar group attached to the amino acid unit, wherein the polar group comprises a polymer unit, optionally a sugar unit, and optionally a carboxyl unit, wherein said polymer unit comprises:
(i) an optionally substituted polyamide comprising a formula 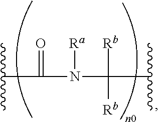
or a stereoisomer thereof, wherein each R^{a} is independently H or C₁₋₆ alkyl and each R^{b} is independently H or C₁₋₆ alkyl, and n0 is independently 2-26;
(ii) a substituted polyether comprising a formula 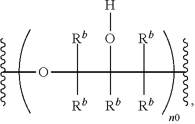
or a stereoisomer thereof, wherein each R^{b} is independently H or C₁₋₆ alkyl, and n0 is independently 2-26; or
(iii) combinations thereof; or
(VI) the linker compound comprises:
(a) the linker unit, which has from 1 to 4 attachment sites for the drug unit and having one of following structures (i) or (ii): 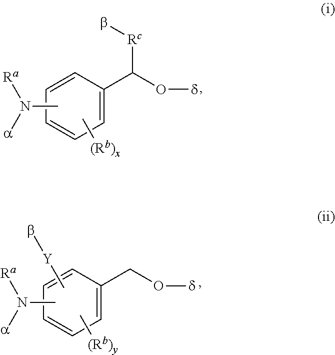
(b) the at least one polar group, each comprises a polymer unit, and
(c) the stretcher group, which has an attachment site for the bispecific binding agent;
wherein:
α—is an attachment site to the amino acid unit, the amino acid unit being an enzyme-cleavable group;
β—is an attachment site to the at least one polar group;
δ—is H, an attachment site to the drug unit, or an attachment site to a linking group attached to the drug unit;
the polymer unit comprises a polyamide, a polyether, or a combination thereof, wherein the polyether comprises a hydroxyl group, a polyhydroxyl group, a sugar group, a carboxyl group, or combinations thereof;
each R^{a} independently is H or C₁-C₆ alkyl;
each R^{b} independently is halo, C₁₋₆ alkyl, an attachment site to the drug unit, or an attachment site to the at least one polar group;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl, a group containing triazolyl, or combinations thereof.

26-30. (canceled)

31. The conjugate of claim 24, wherein the at least one polar group comprises at least one sugar unit having following formula: 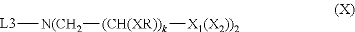
or a stereoisomer or salt thereof, wherein:
each X is independently selected from NH and O;
each R is independently selected from hydrogen, acetyl, a monosaccharide, a disaccharide, and a polysaccharide;
each X₁ is independently selected from CH₂ and C(O);
each X₂ is independently selected from H, OH and OR;
k is 1 to 10; and
L3 is a point of attachment to a remainder of the polar group.

32. The conjugate of claim 31, wherein the at least one polar group comprises at least one sugar unit having one of the following structures (XII) or (XIII): 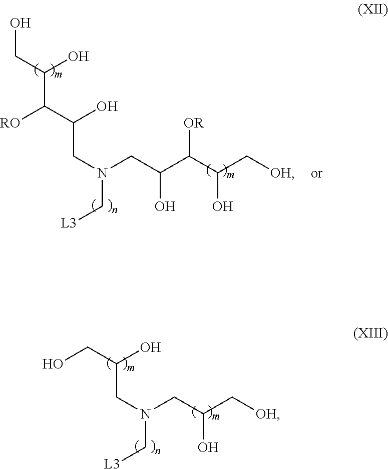
or a stereoisomer or salt thereof, wherein:
each R is independently selected from hydrogen, a monosaccharide, a disaccharide and a polysaccharide;
m is 1 to 8; and
n is 0 to 4.

33. The conjugate of claim 31, wherein the polar group has a formula of: 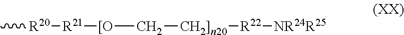
or a stereoisomer a salt thereof, wherein:
R²⁰ is an attachment group to site β or to site R^{b}, or to the amino acid unit, the amino acid unit being an enzyme-cleavable group;
R²¹ and R²² are each, independently, a bond or C₁-C₃ alkylene;
R²⁴ and R²⁵ are each independently selected from a H; polyhydroxyl group; substituted polyhydroxyl group; —C(O)-polyhydroxyl group; substituted —C(O)-polyhydroxyl group;
substituted C₁-C₈alkyl; substituted —C(O)—C₁-C₈alkyl; a chelator; and —C(O)—R²⁸,
wherein R²⁸ is the sugar unit of formula (XII) or (XIII), provided that R²⁴ and R²⁵ are not both H; and
n20 is 2 to 26; or 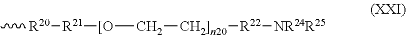
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene;
one of R²⁴ and R²⁵ is selected from a H; polyhydroxyl group; substituted polyhydroxyl group; —C(O)-polyhydroxyl group; substituted —C(O)-polyhydroxyl group; substituted C₁-C₈alkyl; substituted —C(O)—C₁-C₈alkyl; a chelator; and —C(O)—R²⁸, wherein R²⁸ is the sugar unit of formula (XII) or (XIII); and the other of R²⁴ and R²⁵ is a polyethylene glycol, optionally having 1 to 24 ethylene glycol subunits; and
n20 is 2 to 26; or 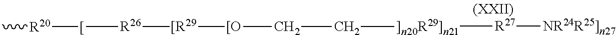
or a stereoisomer or salt thereof, wherein:
R²⁰ is an attachment group to site β or to site R^{b}, or to the enzyme-cleavable group;
R²⁶ and R²⁷ are each optional and are, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
one of R²⁴ and R²⁵ is selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is the sugar unit of formula (XII) or (XIII); and the other of R²⁴ and R²⁵ is selected from H;
polyhydroxyl group; substituted polyhydroxyl group; —C(O)-polyhydroxyl group;
substituted —C(O)-polyhydroxyl group, wherein R²⁸ is the sugar unit of formula (XII) or (XIII); and polyethylene glycol, optionally having 1 to 24 ethylene glycol subunits, provided that R²⁴ and R²⁵ are not both H;
each R²⁹ is optional and independently selected from —C(O)—, —NH—, —C(O)—C₁-C₆ alkylene-, —NH—C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
n20 is 2 to 26;
n21 is 1 to 4; and
n27 is 1 to 4, or 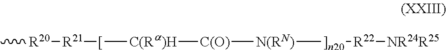
or a stereoisomer or salt thereof, wherein:
R²⁰ is an attachment group to site β or to site R^{b}, or to the enzyme-cleavable group;
R²¹ is a bond or C₁-C₃ alkylene, —C₁-C₃alkylene-[O—CH₂—CH₂—]ₙ₂₀, —[CH₂—CH₂—O]ₙ₂₀—C₁-C₃alkylene-, or —C₁-C₃alkylene-[O—CH₂—CH₂—]ₙ₂₀—C(O)—;
R²² is C₁-C₃ alkylene, —C₁-C₃alkylene-[O—CH₂—CH₂—]ₙ₂₀, —[CH₂—CH₂—O]ₙ₂₀—C₁-C₃alkylene-, or —C₁-C₃alkylene-[O—CH₂—CH₂—]ₙ₂₀—C(O)—;
each R″ is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group;
substituted C₁-C₈ alkyl; substituted —C(O)—C₁-C₈ alkyl; a chelator; and —C(O)—R²⁸,
wherein R²⁸ is the sugar unit of formula (XII) or (XIII), provided that R²⁴ and R²⁵ are not both H; and
each n20 is independently 2 to 26, or 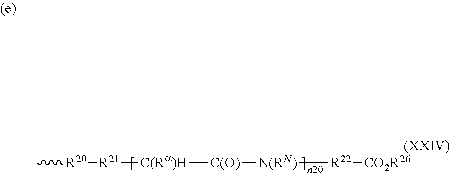
or a stereoisomer or salt thereof, wherein:
R²⁰ is an attachment group to site 3 or to site R^{b}, or to the enzyme-cleavable group;
R²¹ and R²² are each independently a bond, C₁-C₃ alkylene, or —C₁-C₃alkylene[O—CH₂—CH₂—]ₙ₂₀;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group;
substituted C₁-C₈alkyl; substituted —C(O)—C₁-C₈alkyl; a chelator; and —C(O)—R²⁸,
wherein R²⁸ is the sugar unit of formula (XII) or (XIII), provided that R²⁴ and R²⁵ are not both H;
R²⁶ is H or C₁-C₄ alkyl; and
each n20 is independently 2 to 26,
with the proviso that at least one R^{α} or R^{N} is —R²²—NR²⁴R²⁵; 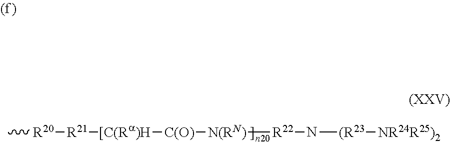
or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, or —C₁-C₃alkylene-[O—CH₂—CH₂—]ₙ₂₀;
each R^{α} is independently H or —R²²—NR²⁴R²⁵;
each R^{N} is independently H or C₁-C₆ alkyl;
each R²³ is independently C₁-C₆ alkylene;
R²⁴ and R²⁵ are each independently selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈alkyl; a chelator; and —C(O)—R²⁸,
wherein R²⁸ is the sugar unit of formula (XII) or (XIII), provided that R²⁴ and R²⁵ are not both H; and
each n20 is independently 2 to 26; 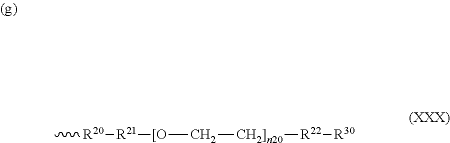
or a stereoisomer or salt thereof, wherein:
R²⁰ is an attachment group to site β to site R^{b}, or to the amino acid unit, the amino acid unit being an enzyme-cleavable group;
R²¹ and R²² are each independently, a bond or C₁-C₃ alkylene groups;
R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)-alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle, or optionally substituted heteroaryl; and
n20 is 2 to 26; 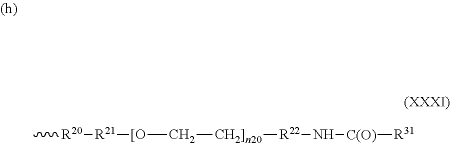
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle, or optionally substituted heteroaryl; and
n20 is 2 to 26; 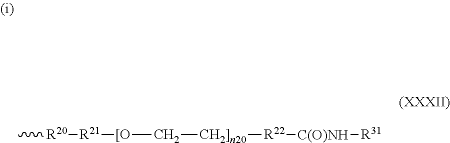
or a stereoisomer or salt thereof, wherein:
R²⁰ is an attachment group to site β or to site R^{b}, or to the enzyme-cleavable group;
R²¹ and R²² are each independently a bond or C₁-C₃ alkylene groups;
R³¹ is a branched polyethylene glycol chain, each branch, independently, having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle, and optionally substituted heteroaryl; and
n20 is 2 to 26; 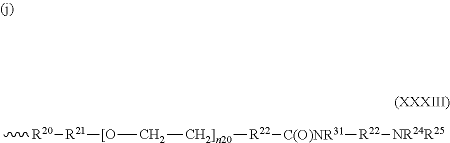
or a stereoisomer or salt thereof, wherein:
R²⁰ is an attachment group to site β or to site R^{b}, or to the enzyme-cleavable group;
R³¹ is H or R²²—NR²⁴R²⁵;
R²¹ and R²² are each independently a bond or C₁-C₃ alkylene groups;
R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H; and
n20 is 2 to 26; 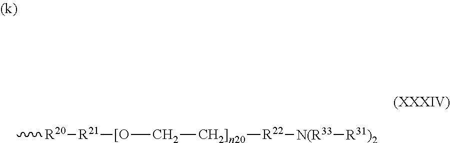
or a stereoisomer or salt thereof, wherein:
R²⁰ is an attachment group to site β or to site R^{b}, or to the enzyme-cleavable group;
R²¹ and R²² are each independently a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, C₁-C₃ alkylene-C(O), —C(O)—C₁-C₃ alkylene, or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle, or optionally substituted heteroaryl; and
n20 is 2 to 26; 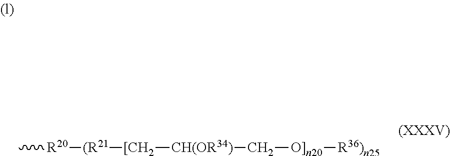
or a stereoisomer or salt thereof, wherein:
R²⁰ is an attachment group to site β or to site R^{b}, or to the enzyme-cleavable group;
each R²¹ is independently a bond, —O— or C₁-C₃ alkylene group;
each R³⁴ is independently H, —[CH₂—CH(OH)—CH₂—O]ₙ₂₀—R³⁶, —C(O)—NR²⁴R²⁵, or —C(O)N(R^{N})—C₁-C₆alkylene-NR²⁴R²⁵;
R^{N} is H or C₁-C₄alkyl;
R²⁴ and R²⁵ are each independently selected from a H; polyhydroxyl group; or
substituted polyhydroxyl group, provided that both R²⁴ and R²⁵ are not H;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)—NR²⁴R²⁵, —C(O)N(R^{N})—C₁-C₆alkylene-NR²⁴R²⁵, C₁-C₆alkylene-C(O)NR²⁴R²⁵, or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; and substituted —C(O)-polyhydroxyl group, provided that both R⁴⁴ and R⁴⁵ are not H;
each n20 is independently 1 to 26; and
n25 is 1 or 2; 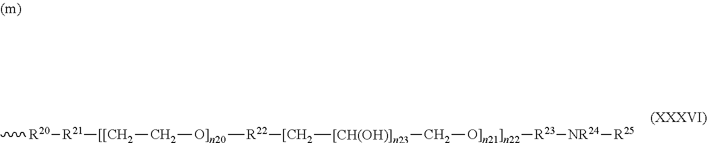
or a stereoisomer or salt thereof, wherein:
R²⁰ is an attachment group to site β or to site R^{b}, or to the enzyme-cleavable group;
R²¹, R²², and R²³ are each independently a bond or C₁-C₃ alkylene group;
R²⁴ and R²⁵ are each independently selected from a H; polyhydroxyl group; substituted polyhydroxyl group; —C(O)-polyhydroxyl group; and substituted —C(O)-polyhydroxyl group, provided that R²⁴ and R²⁵ are not both H;
each n20 is independently 0 to 26, and each n21 is independently 0 to 26, with the proviso that at least one of n20 or n21 is 2 to 26;
n22 is 1 to 5;
each n23 is independently 1 or 2; 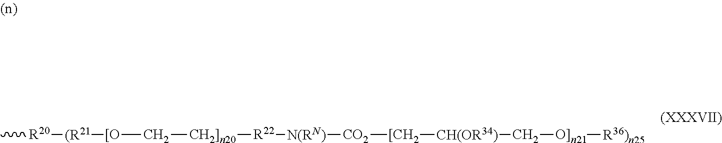
or a stereoisomer or salt thereof, wherein:
R²⁰ is an attachment group to site β or to site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; and
substituted polyhydroxyl group, provided that both R²⁴ and R²⁵ are not H;
each R³⁴ is independently H, —[CH₂—CH(OH)—CH₂—O]ₙ₂₀—R³⁶ or —C(O)N(R^{N})—C₁-C₆alkylene-NR²⁴R²⁵;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)N(R^{N})—C₁-C₆alkylene-NR²⁴R²⁵, C₁-C₆alkylene-C(O)NR²⁴R²⁵, or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; and substituted —C(O)-polyhydroxyl group; provided that both R⁴⁴ and R⁴⁵ are not H;
n20 is 2 to 26;
n21 is 1 to 26; and
n25 is 1 or 2; 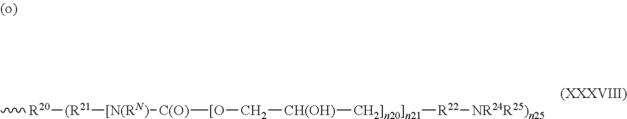
or a stereoisomer or salt thereof, wherein:
R²⁰ is an attachment group to site β or to site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; and
substituted polyhydroxyl group, provided that R²⁴ and R²⁵ are not both H;
n20 is 2 to 26;
n21 is 1 to 4; and
n25 is 1, 2 or 3; 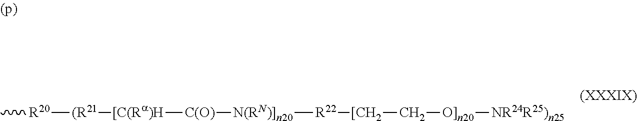
or a stereoisomer or salt thereof, wherein:
R²⁰ is an attachment group to site β or to site R^{b}, or to the enzyme-cleavable group;
R²¹ and R²² are each independently a bond, C₁-C₃ alkylene, —C₁-C₃alkylene-[O—CH₂—CH₂—]ₙ₂₀, —[CH₂—CH₂—O]ₙ₂₀—C₁-C₃alkylene-, or —C₁-C₃alkylene-[O—CH₂—CH₂—]ₙ₂₀—C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group;
substituted —C(O)—C₁-C₈ alkyl; a chelator; —C(O)—R²⁸, wherein R²⁸ is the sugar unit of formula (XII) or (XIII), provided that R²⁴ and R²⁵ are not both H;
each n20 is independently 0 to 26, with the proviso that at least one n20 is 2 to 26; and
n25 is 1 or 2; or 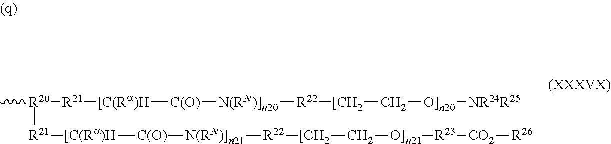
or a stereoisomer or salt thereof, wherein:
R²⁰ is an attachment group to site β or to site R^{b}, or to the enzyme-cleavable group;
R²¹, R²², and R²³ are each independently a bond, C₁-C₃ alkylene, —C₁-C₃alkylene-[O—CH₂—CH₂—]ₙ₂₀, —[CH₂—CH₂—O]ₙ₂₀—C₁-C₃alkylene-, or —C₁-C₃alkylene-[O—CH₂—CH₂—]ₙ₂₀—C(O)—;
each R^{α} is independently H or —R²²—NR²⁴R²⁵;
each R^{N} is independently H, C₁-C₆ alkyl, or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group;
substituted —C(O)—C₁-C₈ alkyl; a chelator; and —C(O)—R²⁸, where R²⁸ is the sugar unit of formula (XII) or (XIII), provided that R²⁴ and R²⁵ are not both H;
R²⁶ is H or C₁-C₆ alkyl;
each n20 is independently 0 to 26, with the proviso that at least one n20 is 2 to 26; and
each n21 is independently 0 to 26, with the proviso that at least one n21 is 2 to 26.

34. (canceled)

35. The conjugate of claim 31, wherein:
(I) the polar group has a formula selected from the following, or a stereoisomer or salt thereof: 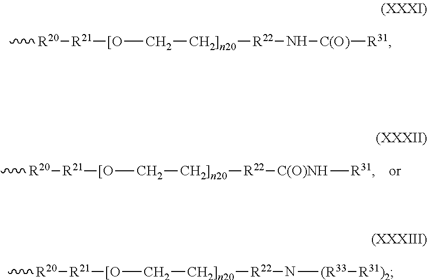
wherein:
R²⁰ is an attachment group to site β or to site R^{b}, or to the amino acid unit, the amino acid unit being an enzyme-cleavable group;
R²¹ and R²² are each independently a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, —C₁-C₃ alkylene-C(O), —C(O)—C₁-C₃ alkylene, or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle, or optionally substituted heteroaryl; the wavy (˜) line indicates an attachment site to R²⁰; and n20 is 2 to 26;
(II) the polar group has a formula: 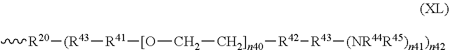
or a stereoisomer or salt thereof, wherein:
R²⁰ is an attachment group to site β or to site R^{b}, or to the amino acid unit, the amino acid unit being an enzyme-cleavable group;
R⁴¹ and R⁴² are each independently a bond or C₁-C₆ alkylene;
each R⁴³ is independently selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein each alkylene is optionally substituted with hydroxyl, SO₃H and/or oxo, R^{a} is H, C₁-C₆ alkyl, a polyhydroxyl group, or a substituted polyhydroxyl group, and one of R⁴⁶ and R⁴⁷ is H or C₁-C₁₂ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each independently H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 2 to 26, provided that R⁴⁴ and R⁴⁵ are not both H;
n40 is 2 to 26;
n41 is 1 to 6; and
n42 is 1 to 6;
(III) the polar group has a formula: 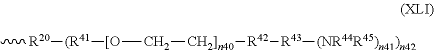
or a stereoisomer or salt thereof, wherein:
R²⁰ is an attachment group to site β or to site R^{b}, or to the amino acid unit, the amino acid unit being an enzyme-cleavable group;
R⁴¹ and R⁴² are each independently a bond or C₁-C₆ alkylene;
R⁴³ is selected from a bond, C₁-C₁₂ alkylene, —OC₁-C₁₂ alkylene, —C(═O)—, —NR^{a}—C₁-C₁₂ alkylene, —C₁-C₁₂ alkylene-N′R^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, C(O)—NR^{a}—C₁-C₁₂ alkylene, -heteroarylene, heteroaryl-C₁-C₁₂ alkylene, heteroaryl-C₁-C₁₂ alkylene-C(O)—, and —C(O)NR⁴⁶R⁴⁷, wherein each alkylene is optionally substituted with hydroxyl, SO₃H and/or oxo, R^{a} is H, C₁-C₆ alkyl, a polyhydroxyl group, or a substituted polyhydroxyl group, and one of R⁴⁶ and R⁴⁷ is H or C₁-C₁₂ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each independently H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
n40 is 1 to 26;
n41 is 1 to 6; and
n42 is 1 to 6;
(IV) the polar group has a formula: 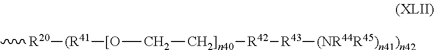
or a stereoisomer or salt thereof, wherein:
R²⁰ is an attachment group to site β or to site R^{b}, or to the amino acid unit, the amino acid unit being an enzyme-cleavable group;
R⁴¹ and R⁴² are each independently a bond or C₁-C₃ alkylene;
R⁴³ is selected from a bond, C₁-C₆ alkylene, —OC₁-C₁₂ alkylene, —C(═O)—, —NR^{a}—C₁-C₁₂ alkylene, —C₁-C₆ alkylene-NR^{a}—, —C(O)—C₁-C₆ alkylene, —C₁-C₆ alkylene-C(O)—, —NR^{a}—C₁-C₆ alkylene-C(O)—, —C(O)—C₁-C₆ alkylene-NR^{a}—, —NR^{a}—C(O)—NR^{a}—, —NR^{a}—C(O)—, —NR^{a}—C(O)—C₁-C₆ alkylene, —C(O)—NR^{a}—C₁-C₁₂ alkylene, -heteroarylene, heteroaryl-C₁-C₆ alkylene, heteroaryl-C₁-C₆ alkylene-C(O)—, and —C(O)NR⁴⁶R⁴⁷, wherein each alkylene is optionally substituted with hydroxyl, SO₃H, and/or oxo, R^{a} is H, C₁-C₆ alkyl, a polyhydroxyl group, or a substituted polyhydroxyl group and one of R⁴⁶ and R⁴⁷ is H or C₁-C₆ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each independently H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
n40 is 1 to 16;
n41 is 1 to 4; and
n42 is 1 to 4;
(V) the polar group has a formula selected from: 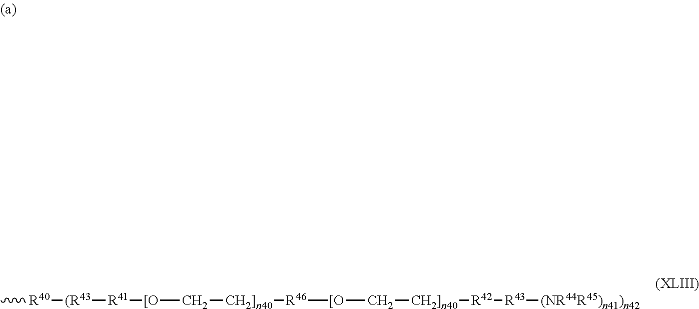
or a stereoisomer or salt thereof, wherein:
R⁴⁰ is an attachment group to site R^{b}, or to the amino acid unit, the amino acid unit being an enzyme-cleavable group;
R⁴¹ and R⁴² are each independently, a bond or C₁-C₆ alkylene;
each R⁴³ is independently selected from a bond, C₁-C₁₂ alkylene, —OC₁-C₁₂ alkylene, —C(═O)—, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, C₁-C₁₂alkylene-NH—C(O)—, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each independently H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
each R⁴⁶ is independently selected from —NR⁵⁰—, —NR⁵⁰—C₁-C₆alkylene-NR⁵⁰—, —NR⁵⁰—C(O)—NR⁵⁰—S(O)₂—NR⁵⁰— or —NR⁵⁰—C(O)—C₁₋₆alkylene- each R⁵⁰ is independently selected from H, C₁-C₆ alkyl, or polyhydroxyl group;
each n40 is independently 2 to 26;
n41 is 1 to 6; and
n42 is 1 to 6; 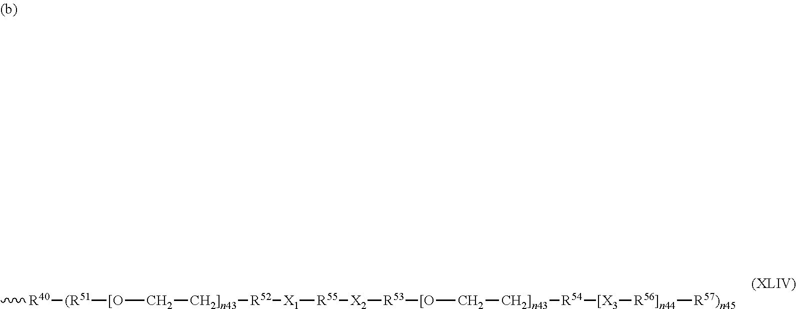
or a stereoisomer or salt thereof, wherein:
R⁴⁰ is an attachment group to site R^{b}, or to the enzyme-cleavable group;
R⁵¹, R⁵², R⁵³, and R⁵⁴ are each independently a bond or C₁-C₆ alkylene;
X₁, X₂, and X₃ are each independently —NR^{N}—C(O)— or —C(O)—NR^{N}—;
each R^{N} independently represent H, C₁-C₆ alkyl, or polyhydroxyl group;
R⁵⁵ and R⁵⁶ each independently represent a bivalent polyhydroxyl group;
R⁵⁷ is H, OH, or C₁-C₆ alkyl;
each n43 is independently 0 to 26, with the proviso that at least one n43 is 1 to 26;
n44 is 0 to 10; and
n45 is 1 or 2; or 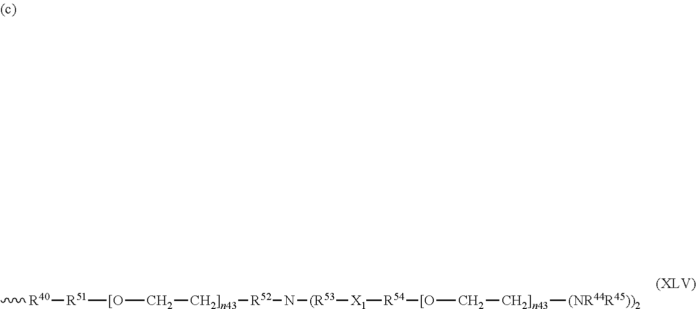
or a stereoisomer or salt thereof, wherein:
R⁴⁰ is an attachment group to site R^{b}, or to the enzyme-cleavable group;
R⁵¹, R⁵³, and R⁵⁴ are each independently a bond or optionally-substituted C₁-C₆ alkylene;
R⁵² is a bond, C₁-C₆ alkylene, —C(O)— or —O—C(O)—;
each X₁ is independently —NR^{N}—C(O)— or —C(O)—NR^{N}—;
each R^{N} independently represent H, C₁-C₆ alkyl, or polyhydroxyl group;
R⁴⁴ and R⁴⁵ are each independently H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; and
each n43 is independently 2 to 26; or
(VI) the polar group has a formula selected from: 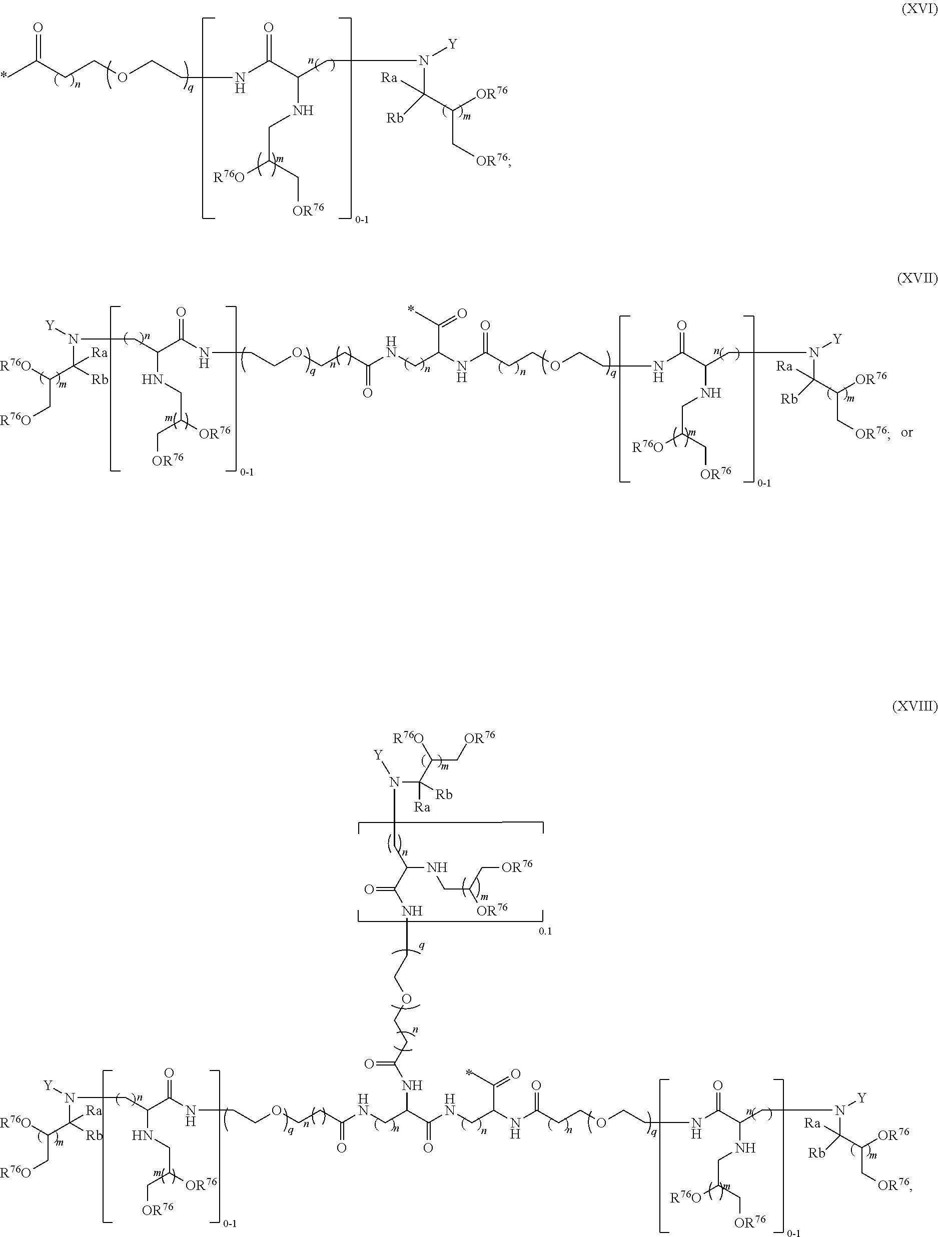
or a stereoisomer or salt thereof, wherein:
each Y is independently R⁷⁶ or 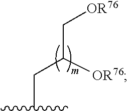
each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 2-26;
each m is independently 1 to 4;
each n is independently 1 to 4;
each v is independently 1 to 6; and
each * is an attachment site to R^{b}, or to the amino acid unit, the amino acid unit being an enzyme-cleavable group.

36-39. (canceled)

40. The conjugate of claim 35, wherein the polar group has one of following structures prior to attachment to the enzyme-cleavable group and/or to the linker unit: 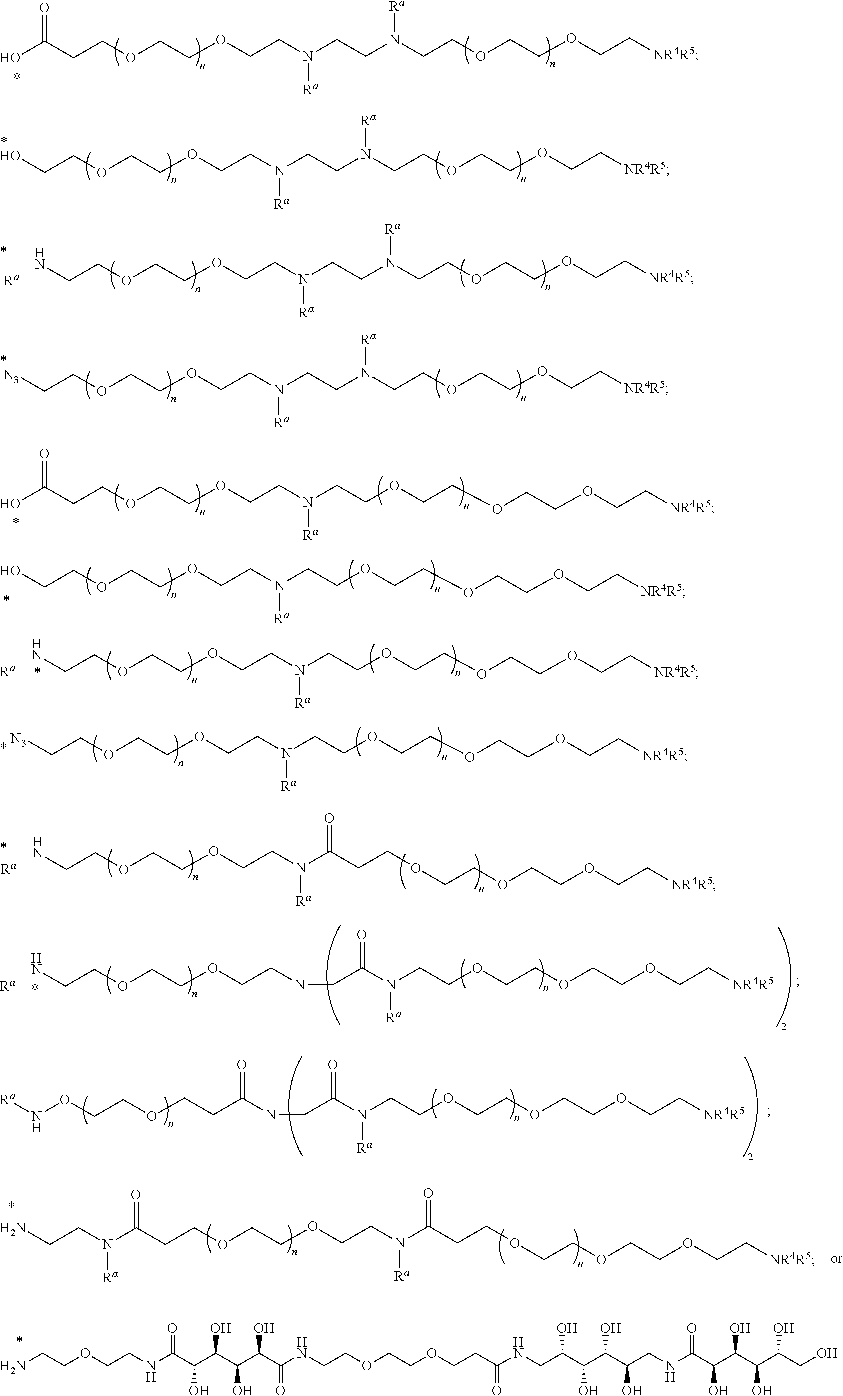
wherein:
(*) indicates the attachment site to site R^{b}, or to the enzyme-cleavable group;
each R is independently H, alkyl, or polyhydroxyl group;
R⁴ and R⁵ are each independently H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴ and R⁵ are not both H; and
each n is independently 1 to 12.

41. (canceled)

42. The conjugate of claim 31, wherein the polar group comprises at least one carboxyl unit having the following formula: 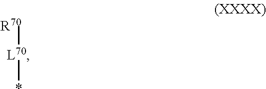
or a stereoisomer or salt thereof, wherein:
(a)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the amino acid unit, the amino acid unit being an enzyme-cleavable group, or to a remainder of the polar group;
R⁷⁰ is ˜NR⁷¹(R⁷²-R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is a bond or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino, and/or amide; or
(b)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the polar group;
R⁷⁰ is ˜NR⁷¹(R⁷⁵—(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino, and/or amide; or
(c)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the polar group;
R⁷⁰ is —N(R⁷⁴-R⁷³)(R⁷²-R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino, and/or amide.

43. The conjugate of claim 42, comprising:
(I) a formula selected from the following: 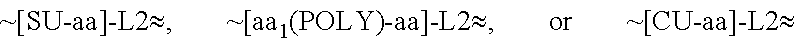
wherein square brackets indicate the amino acid unit, each aa is an optional subunit of the amino acid unit, L2 is the linker unit, each wavy line (˜) indicates an attachment site for the stretcher group; aa₁(POLY) is a polymer unit attached to the subunit of the amino acid unit, SU is the sugar unit attached to the subunit of the amino acid unit or to the linker unit, and CU is the carboxyl unit attached to the subunit of the amino acid unit or to the linker unit; and the double wavy (≈) line indicates an attachment site for the drug unit, wherein aa and aa₁ are independently selected from alpha, beta and gamma amino acids and derivatives thereof;
(II) a formula selected from the following: 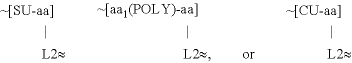
wherein square brackets indicate the amino acid unit, each aa is a subunit of the amino acid unit, L2 is the linker unit attached to a side chain of aa, the wavy line (˜) indicates an attachment site for the stretcher group; aa₁(POLY) is a polymer unit attached to aa, SU is the sugar unit attached to aa, CU is the carboxyl unit attached to aa, and the double wavy (≈) line indicates an attachment site for the drug unit; wherein aa and aa₁ are independently selected from alpha, beta and gamma amino acids and derivatives thereof;
(III) a formula selected from the following: 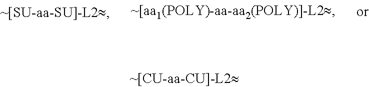
wherein square brackets indicate the amino acid unit, aa is an optional subunit of the amino acid unit, L2 is the linker unit, the wavy line (˜) indicates an attachment site for the stretcher group; each of aa₁(POLY) and aa₂(POLY) is a polymer unit attached to aa or to other polymer unit; each SU is the sugar unit attached to aa or other sugar unit, each CU is the carboxyl unit attached to aa or to other carboxyl unit, and the double wavy (≈) line indicates an attachment site for the drug unit; wherein aa, aa₁, and aa₂ are independently selected from alpha, beta and gamma amino acids and derivatives thereof; or
(IV) a formula selected from the following: 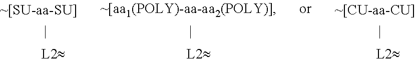
wherein square brackets indicate the amino acid unit, aa is a subunit of the amino acid unit, L2 is the linker unit attached to a side chain of aa, each wavy line (˜) indicates an attachment site for the stretcher group; each of aa₁(POLY) and aa₂(POLY) is a polymer unit attached to aa, each SU is the sugar unit attached to aa; each CU is the carboxyl unit attached to aa; and the double wavy (≈) line indicates an attachment site for the drug unit; wherein each of aa, aa₁, and aa₂ is independently selected from alpha, beta and gamma amino acids and derivatives thereof.

44-46. (canceled)

47. The conjugate of claim 24, wherein the linker unit has the following structure: 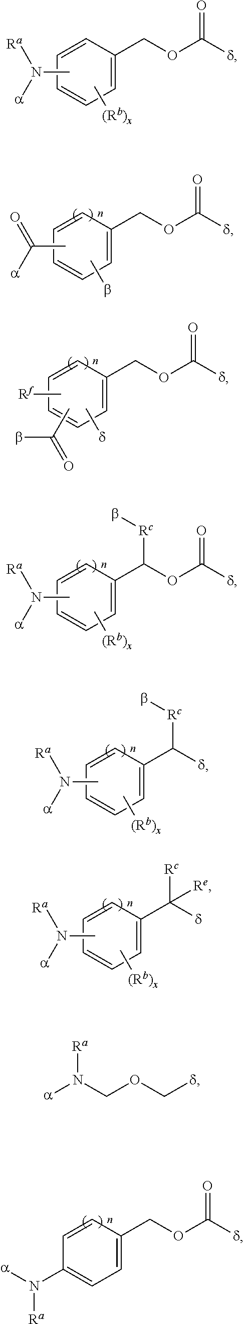 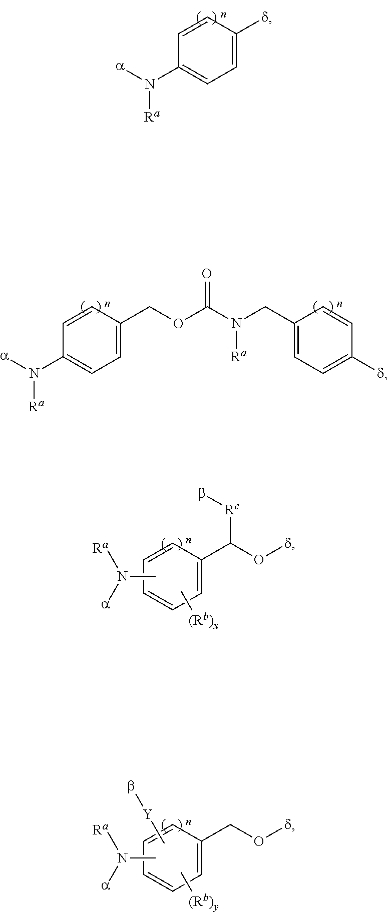
or a stereoisomer or salt thereof, wherein:
α—represents an attachment site to the amino acid unit, when present, or to the stretcher unit;
β—represents an attachment site to the at least one polar group;
δ—represents an attachment site to the drug unit or an attachment site to a linking group attached to the drug unit;
R^{a} is H or C₁₋₆ alkyl;
R^{b} is H, halo, C₁₋₆ alkyl, an attachment site to the drug unit or the linking group attached to the drug unit, or an attachment site to the at least one polar group or a linking group attached to the at least one polar group;
R^{c} is a bond, —C(O)—, —S(O)—, —SO₂—, C₁₋₆ alkylene, C₁₋₆ alkynylene, triazolyl, or combinations thereof;
R^{c} and R^{d} are each independently H, C₁-C₆ alkyl, C₁₋₆ alkylene, C₁₋₆ alkynylene, nitrile group, alkynyl group, nitrogen triyl group, an ester group, substituted triazolyl, substituted amino group, substituted thiol group, substituted silicon group, substituted phosphate group, 3 to 8 aromatic rings, cyclic hydrocarbons, cyclic hydrocarbon heterocycles, aromatic heterocycles, or R^{e} and R^{d} join together to form cycloalkyl, or an attachment site to a linking group attached to the at least one polar group;
R^{f} is an attachment site to the stretcher unit or an attachment site to a linking group attached to the stretcher unit;
Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl, or combinations thereof;
x is 0, 1, 2, 3 or 4;
y is 0, 1, 2 or 3;
n is 0-2.

48. The conjugate of claim 24, wherein the stretcher group is selected from the following: 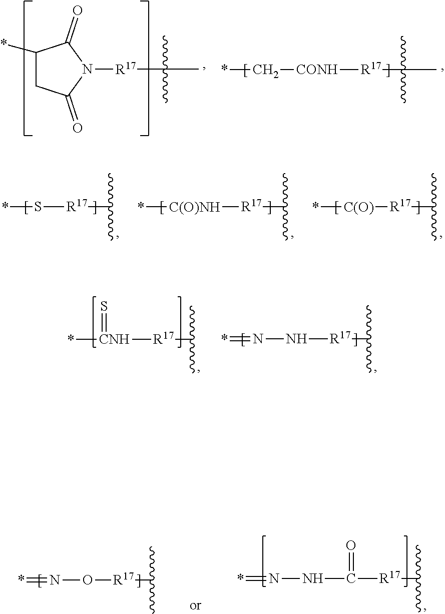
wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀alkylene-C(O)NH—C₁-C₈alkylene-[O—CH₂—CH₂]ₙ—C(O)— (where n is 1 to 26), C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the stretcher group comprises maleimido(C₁-C₁₀ alkylene-C(O)—, maleimido(CH₂OCH₂)ₚ₂(C₁-C₁₀ alkylene)C(O)—, maleimido(C₁-C₁₀alkylene) (CH₂OCH₂)ₚ₂C(O)—, or a ring open form thereof, wherein p2 is from 1 to 26;
and wherein * is an attachment to the binding agent, and the wavy line is an attachment to the amino acid unit, the amino acid unit being an enzyme-cleavable group.

49. The conjugate of claim 48, wherein the stretcher group is selected from the following: 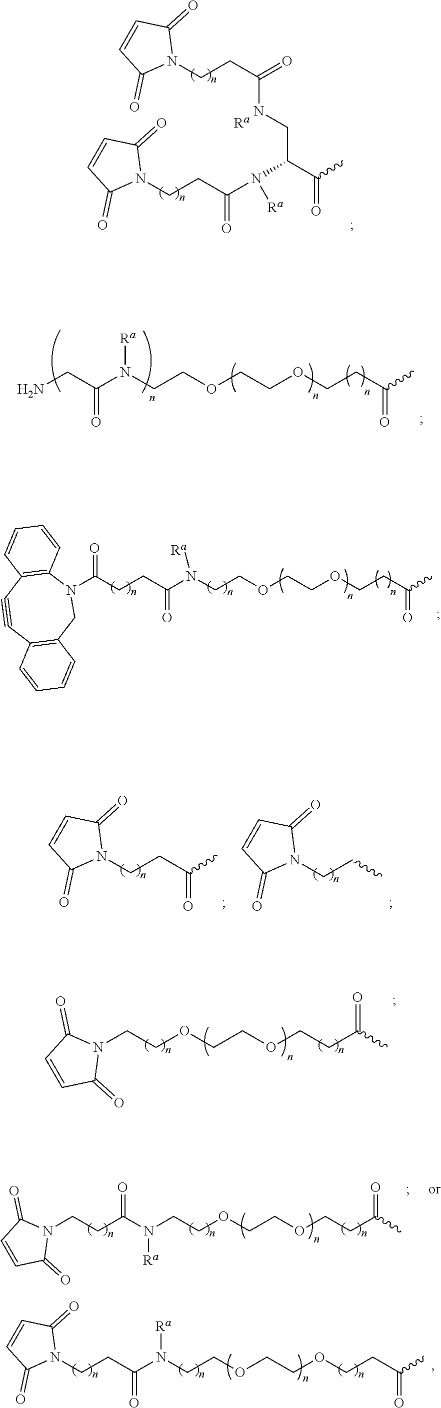
or a stereoisomer thereof, wherein each R^{a} is independently H or C₁₋₆ alkyl, each n is independently 0-12, and the wavy line [symbol] indicates an attachment site of the stretcher group to the amino acid unit, and the attachment site for the targeting unit is on a maleimide, primary amine or alkyne functional group.

50. The conjugate of claim 24, wherein the linker compound has one of the following structures: 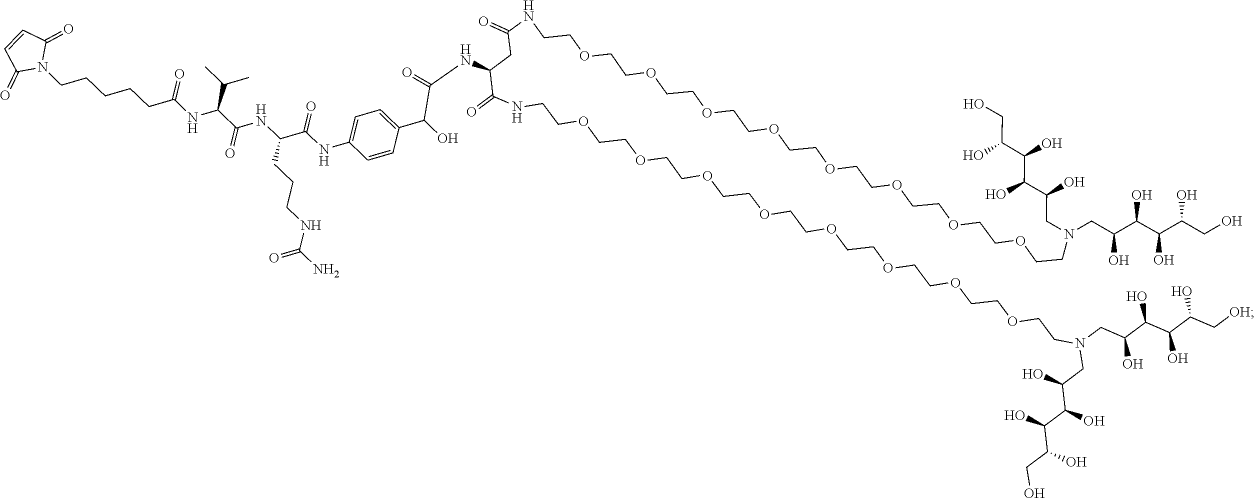 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 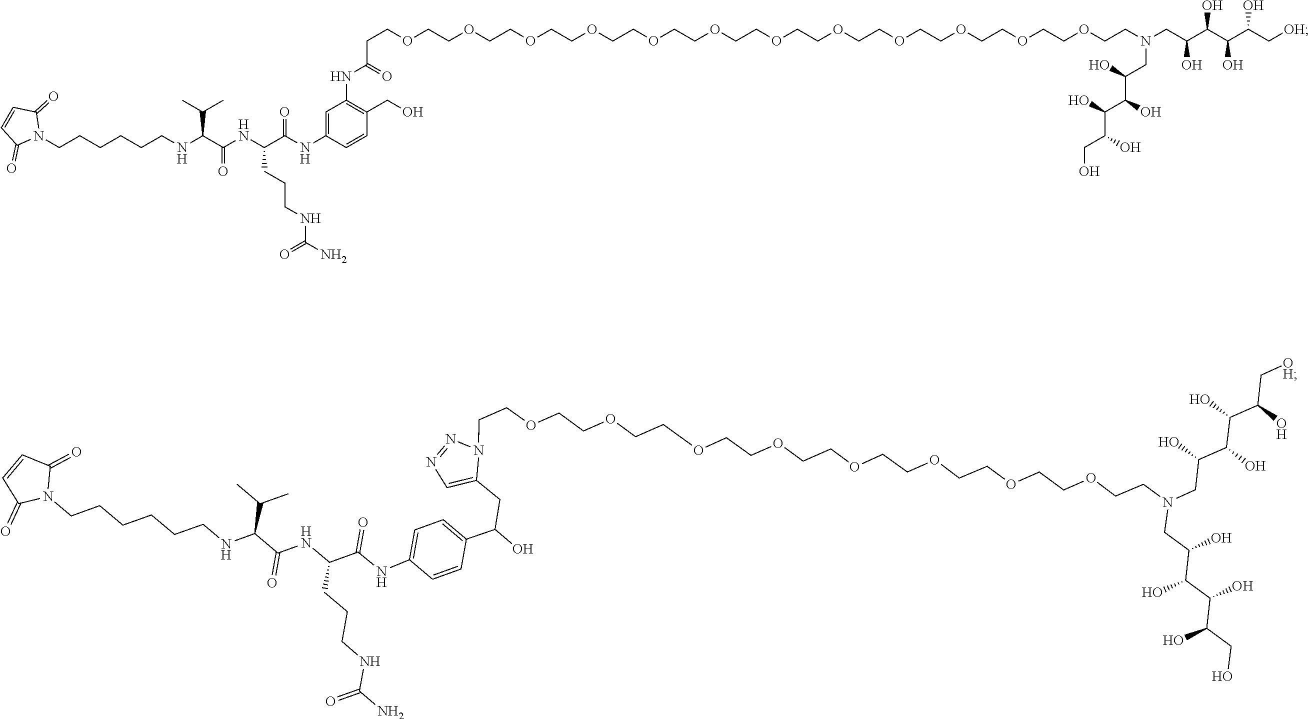 [SMILES: CC(C)[C@H](NCCCCCCN1C(=O)C=CC1=O)C(=O)N[C@@H](CCCNC(N)=O)C(=O)Nc1ccc(CO)c(NC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1 ; CC(C)[C@H](NCCCCCCN1C(=O)C=CC1=O)C(=O)N[C@@H](CCCNC(N)=O)C(=O)Nc1ccc(C(O)Cc2cnnn2CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 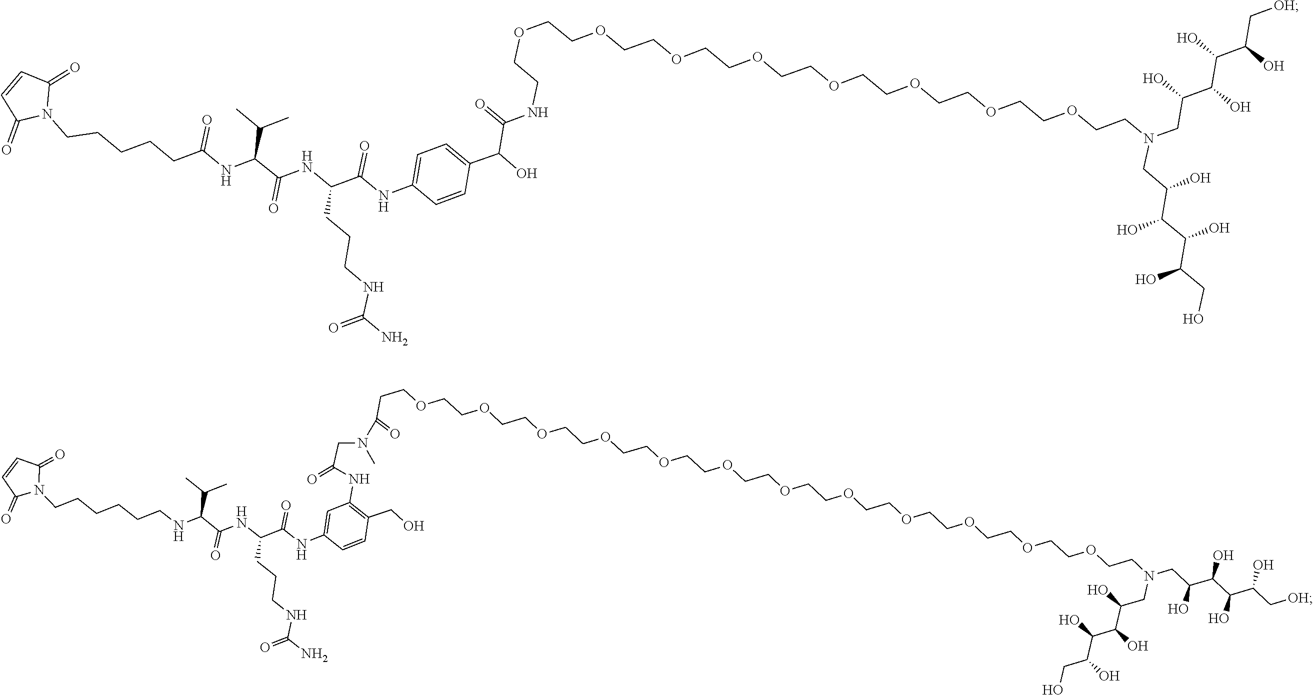 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NCCCCCCN1C(=O)C=CC1=O)C(=O)N[C@@H](CCCNC(N)=O)C(=O)Nc1ccc(CO)c(NC(=O)CN(C)C(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1] 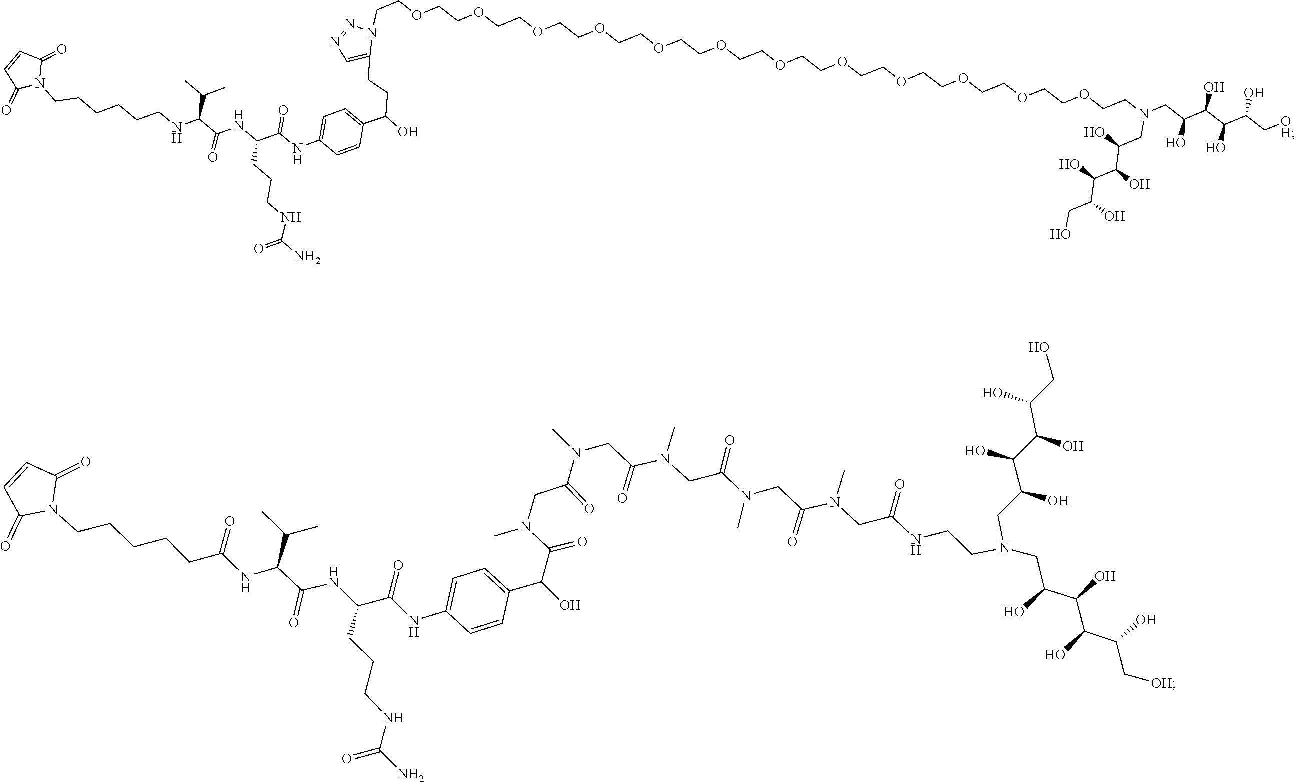 [SMILES: CC(C)[C@H](NCCCCCCN1C(=O)C=CC1=O)C(=O)N[C@@H](CCCNC(N)=O)C(=O)Nc1ccc(C(O)CCc2cnnn2CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1] 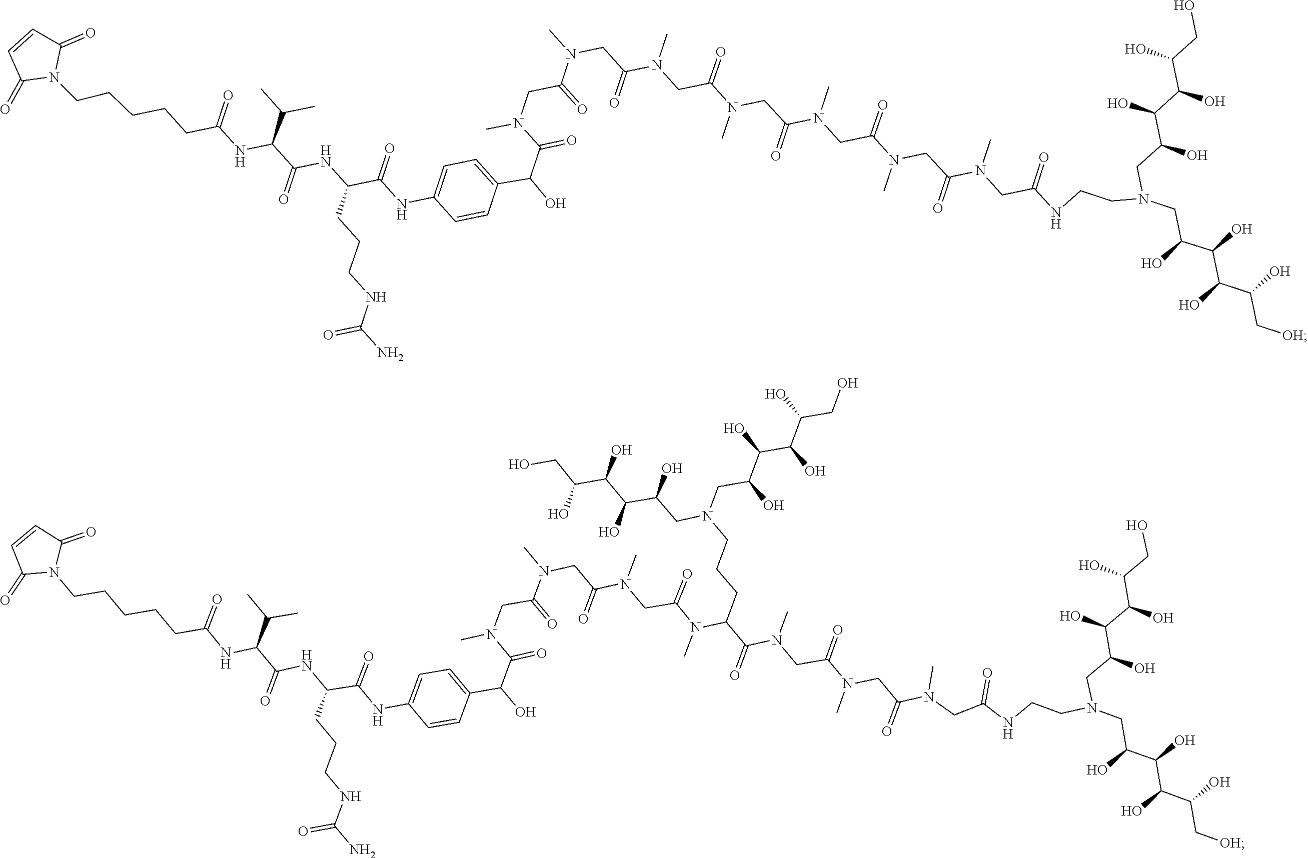 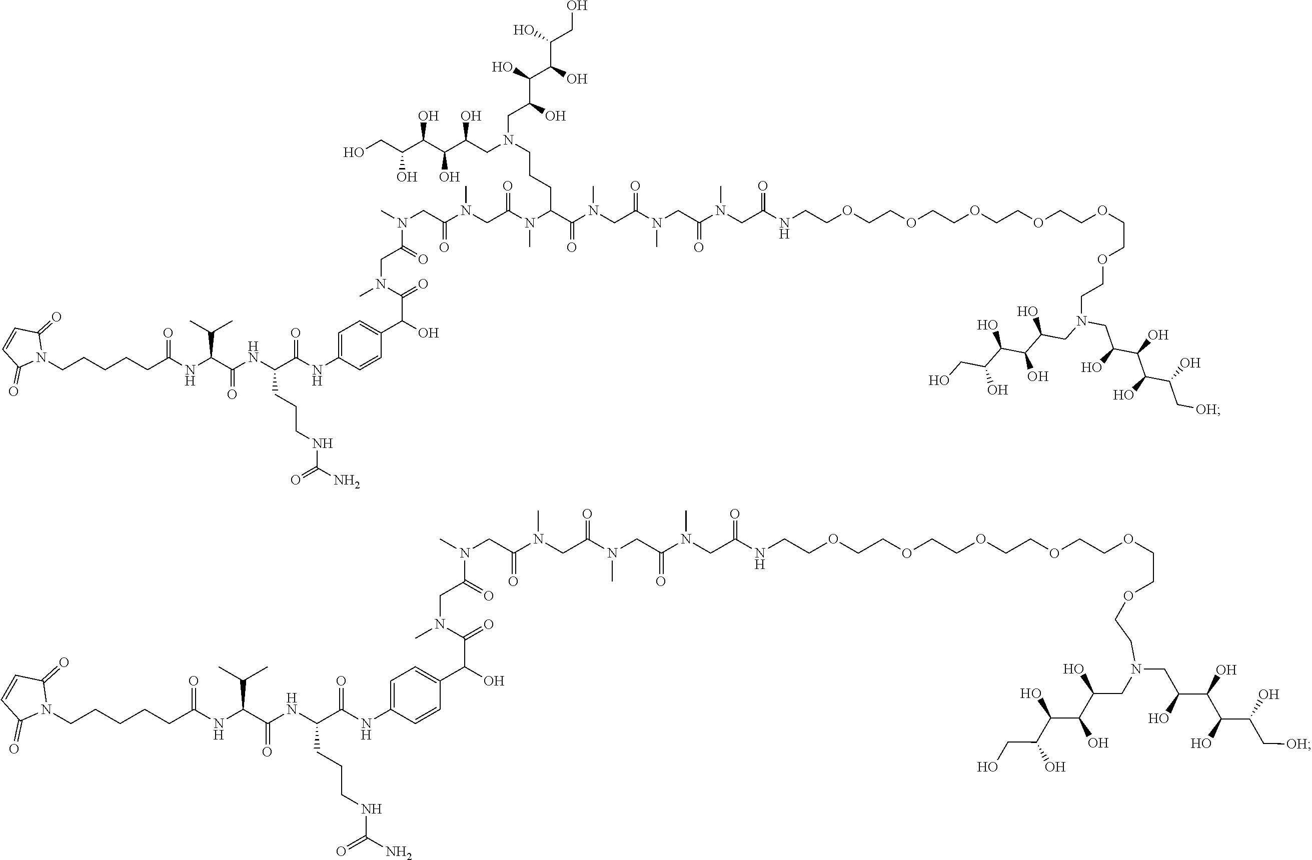 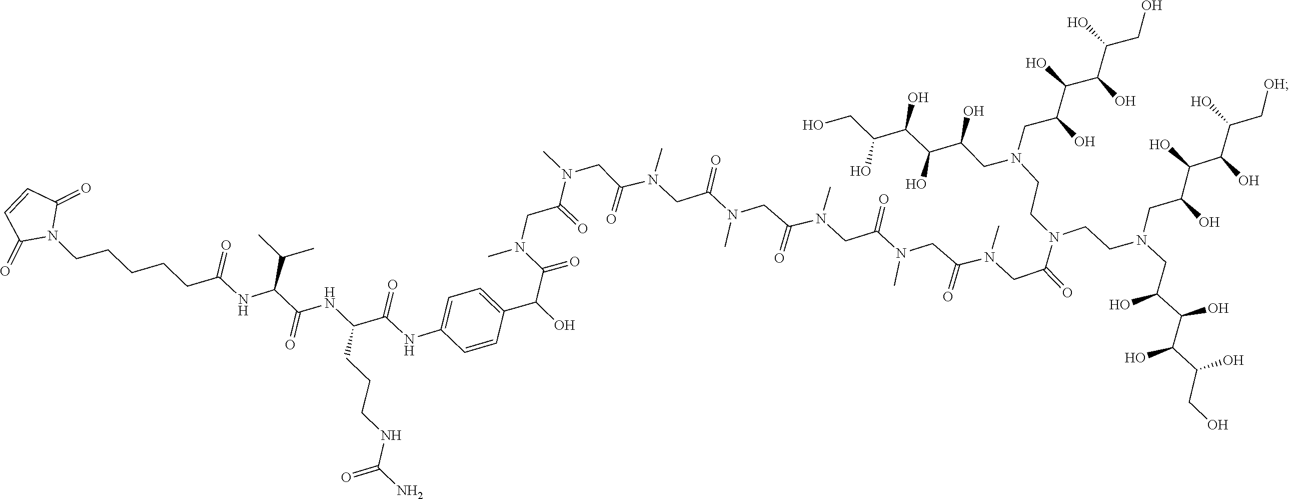 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)cc1] 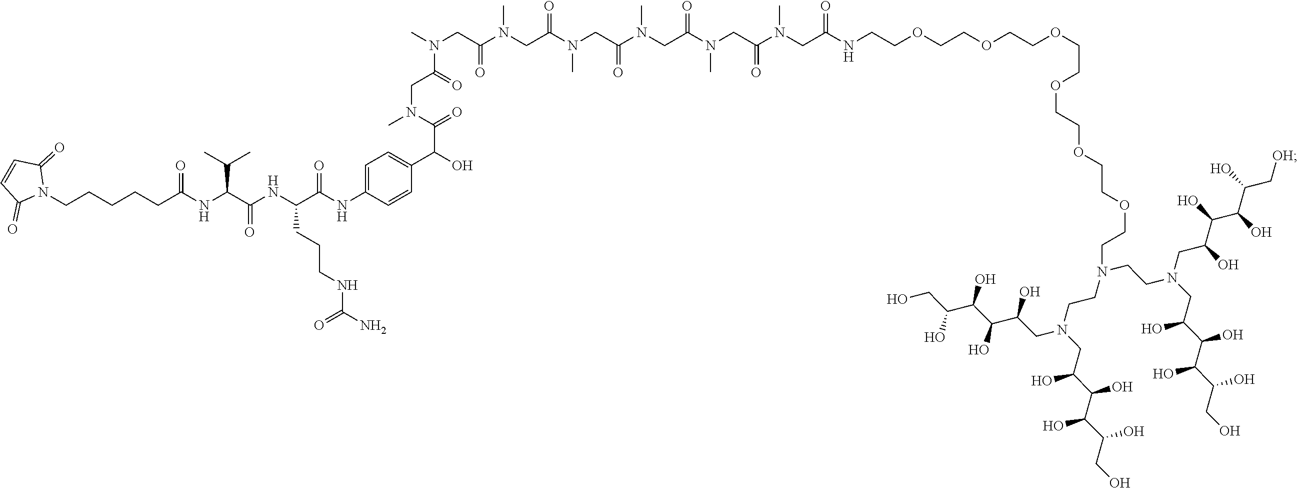 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)CC(=O)N(C)CC(=O)N(C)CC(=O)N(C)CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)cc1] 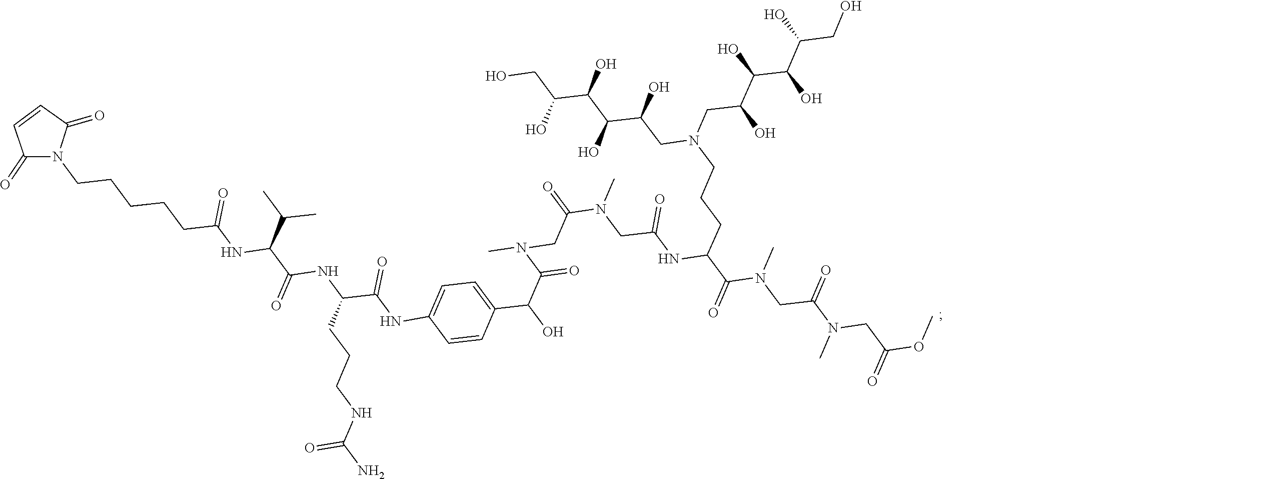 [SMILES: COC(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)C(O)c1ccc(NC(=O)[C@H](CCCNC(N)=O)NC(=O)[C@@H](NC(=O)CCCCCN2C(=O)C=CC2=O)C(C)C)cc1] 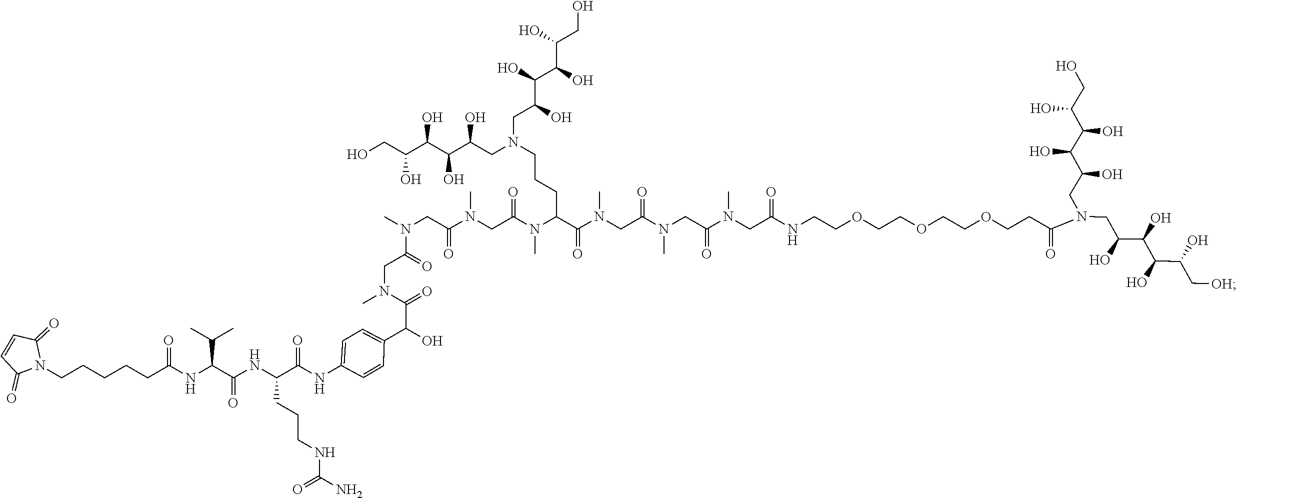 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)cc1] 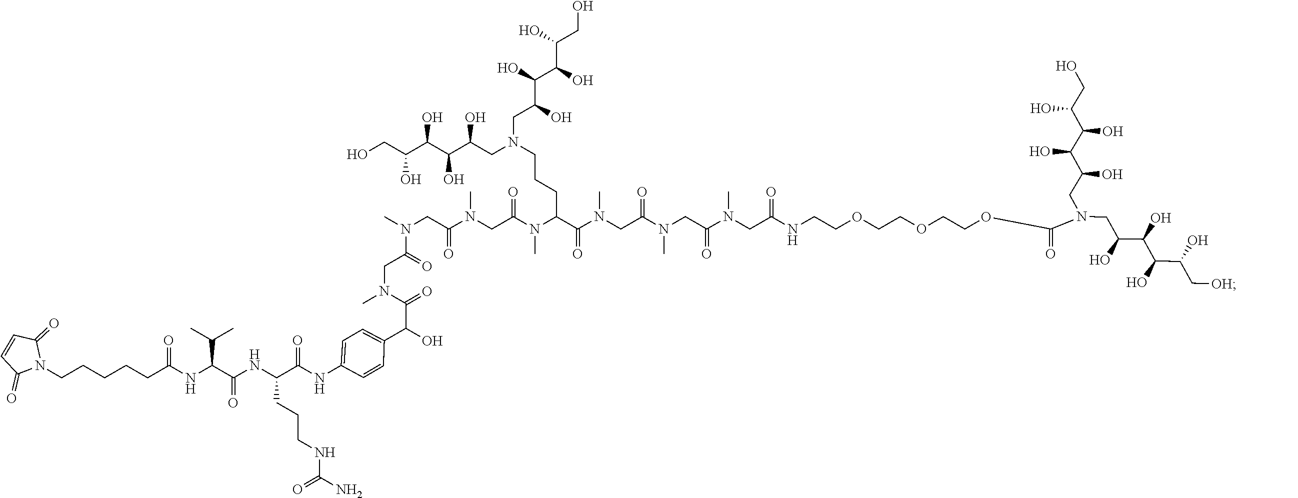 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)N(C[C@H](O)[C@@H](O)[C@H](O)[C@H](O)CO)C[C@H](O)[C@@H](O)[C@H](O)[C@H](O)CO)cc1] 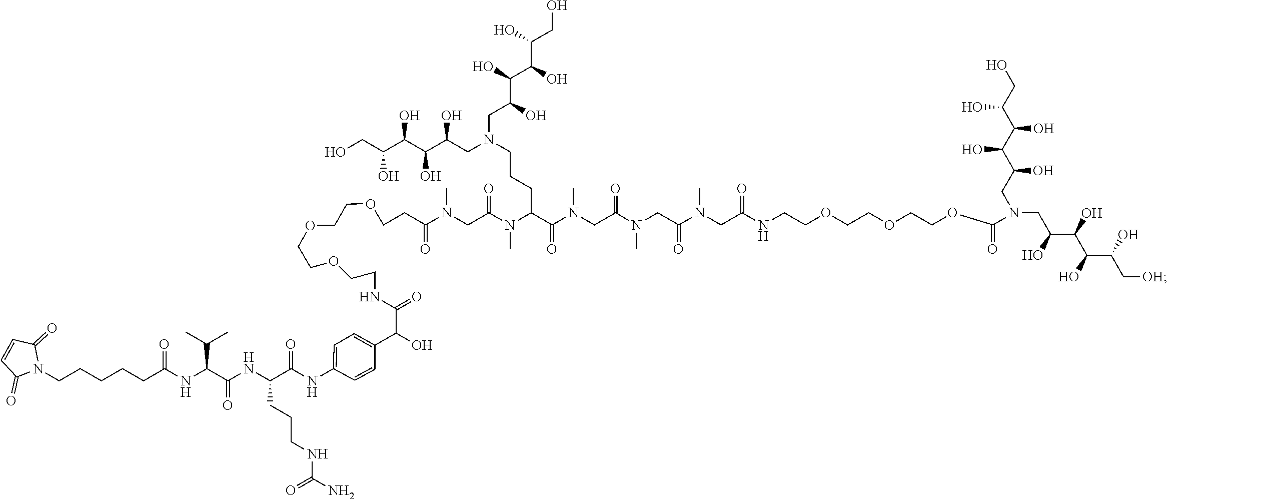 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCOCCC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)N(C[C@H](O)[C@@H](O)[C@H](O)[C@H](O)CO)C[C@H](O)[C@@H](O)[C@H](O)[C@H](O)CO)cc1] 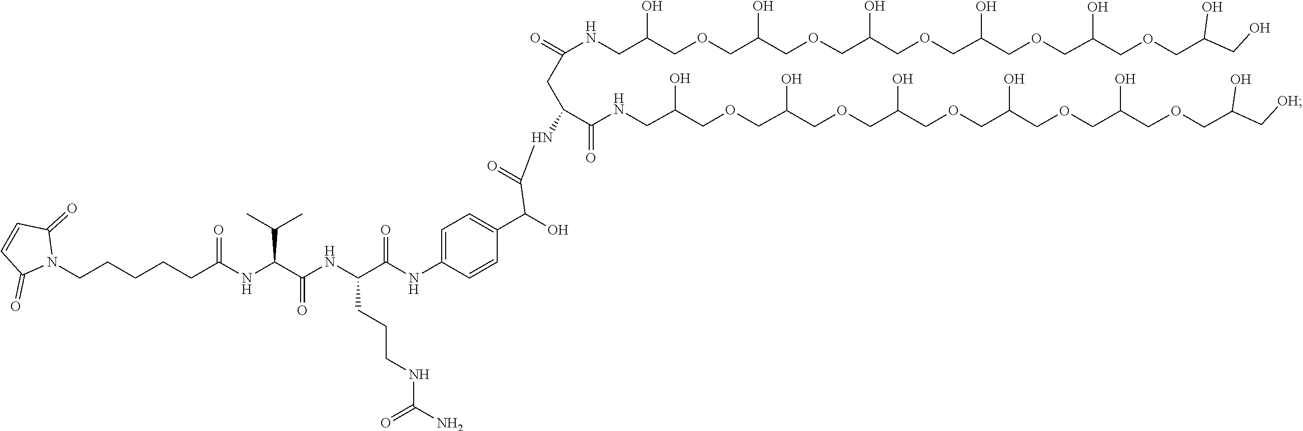 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO)cc1] 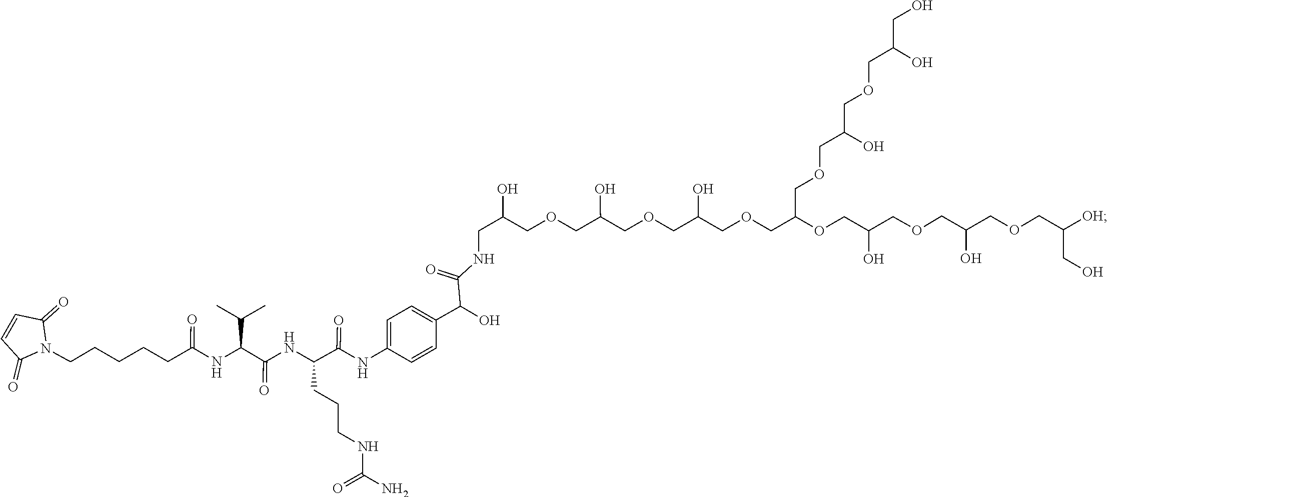 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(COCC(O)COCC(O)CO)OCC(O)COCC(O)COCC(O)CO)cc1] 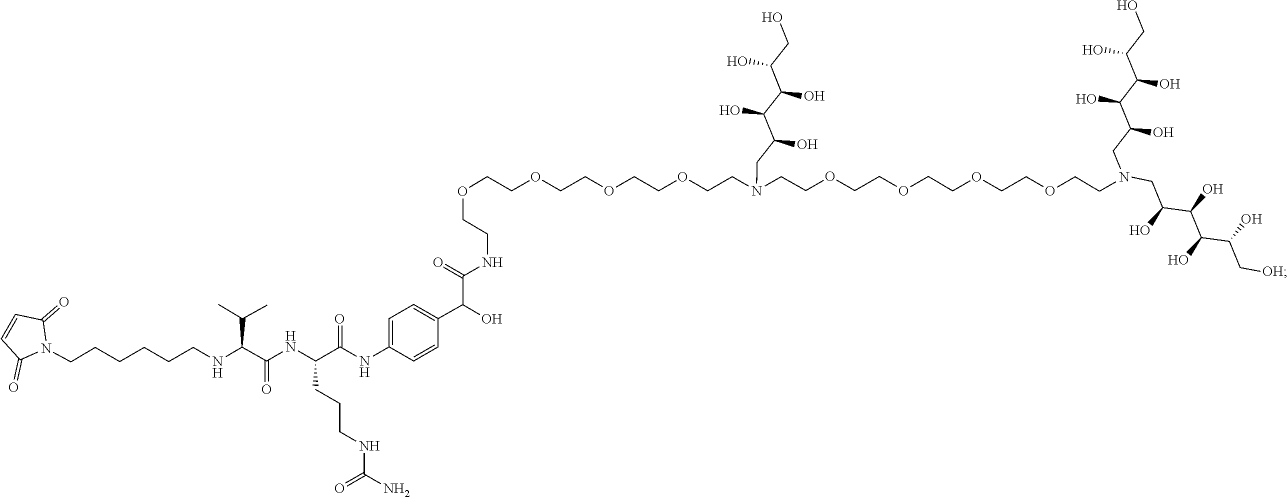 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCOCCOCCN(CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1] 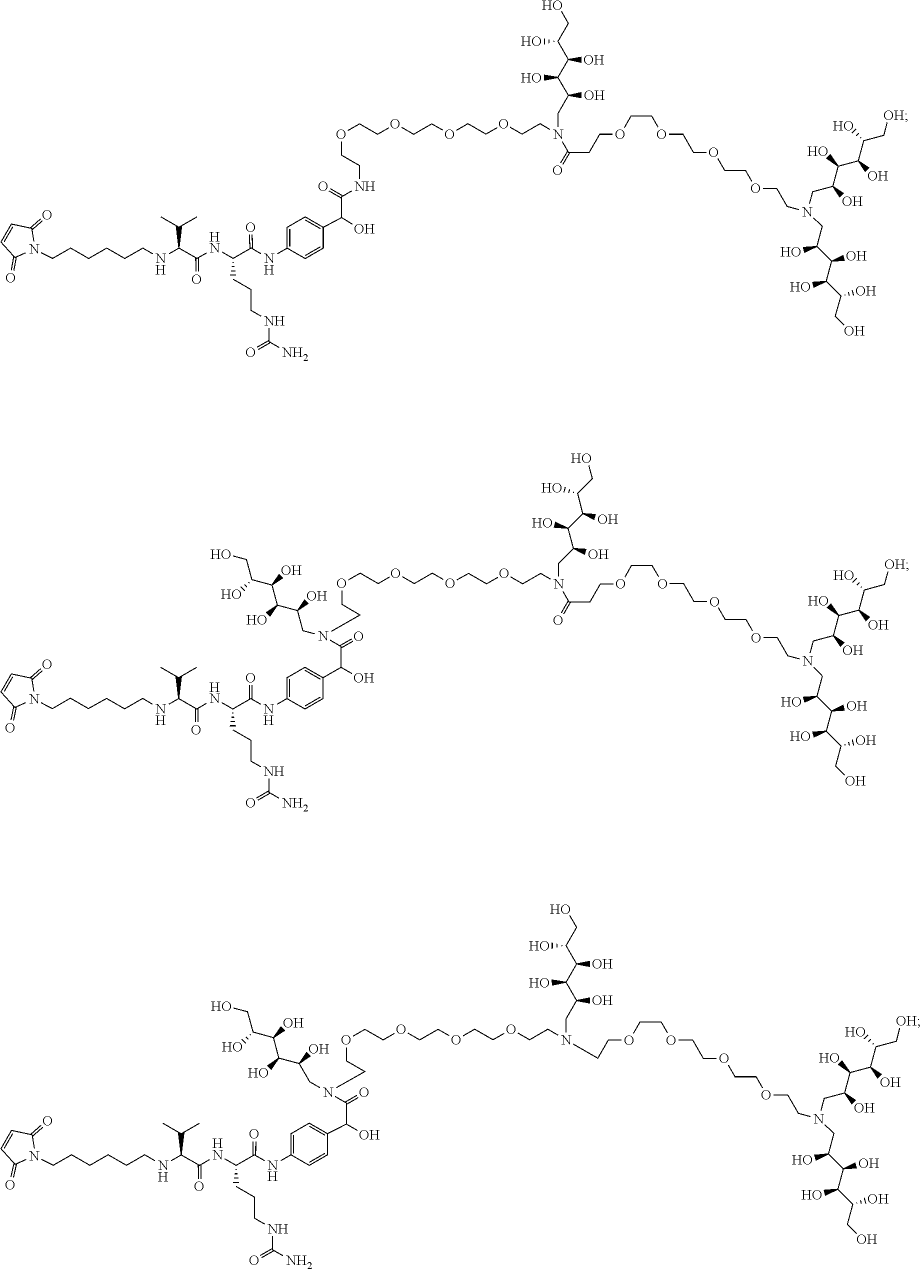 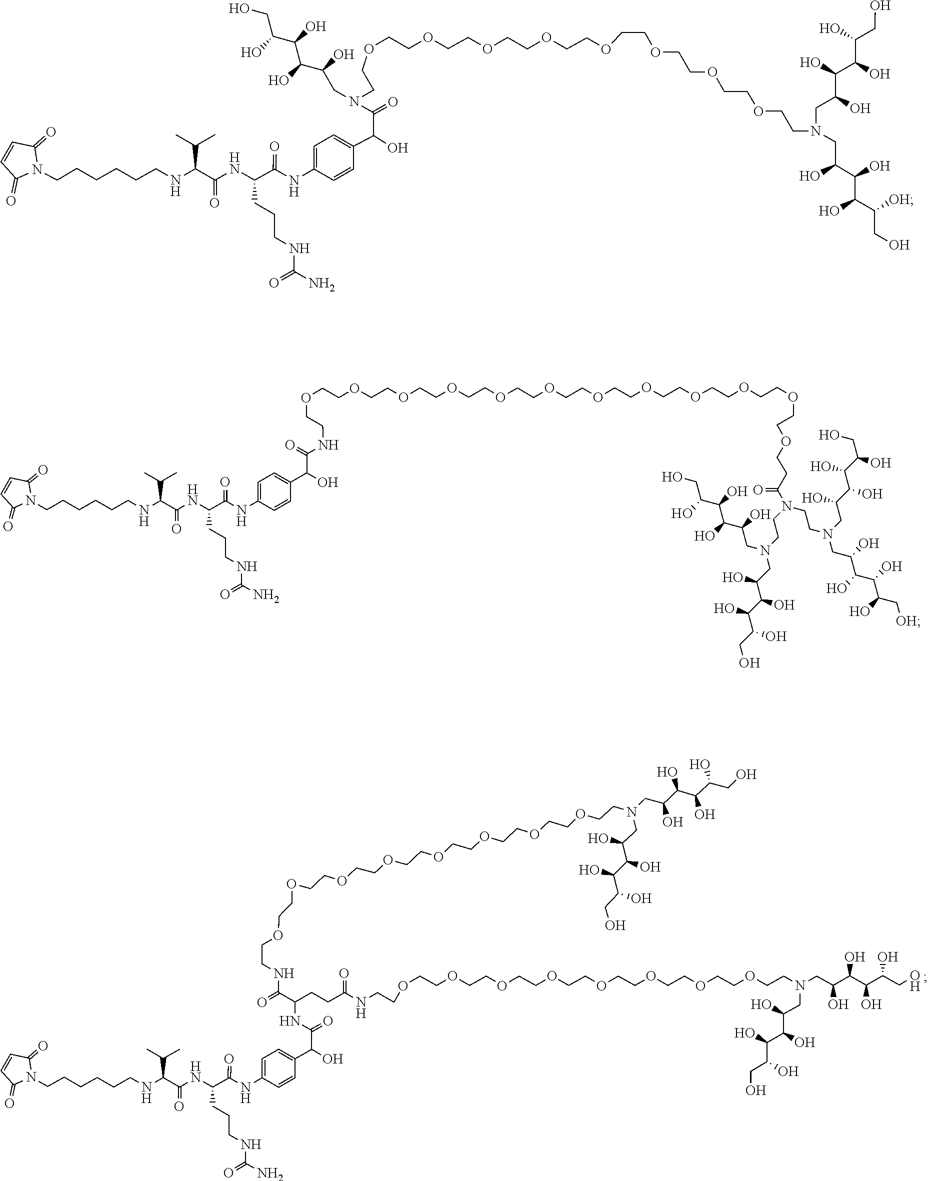 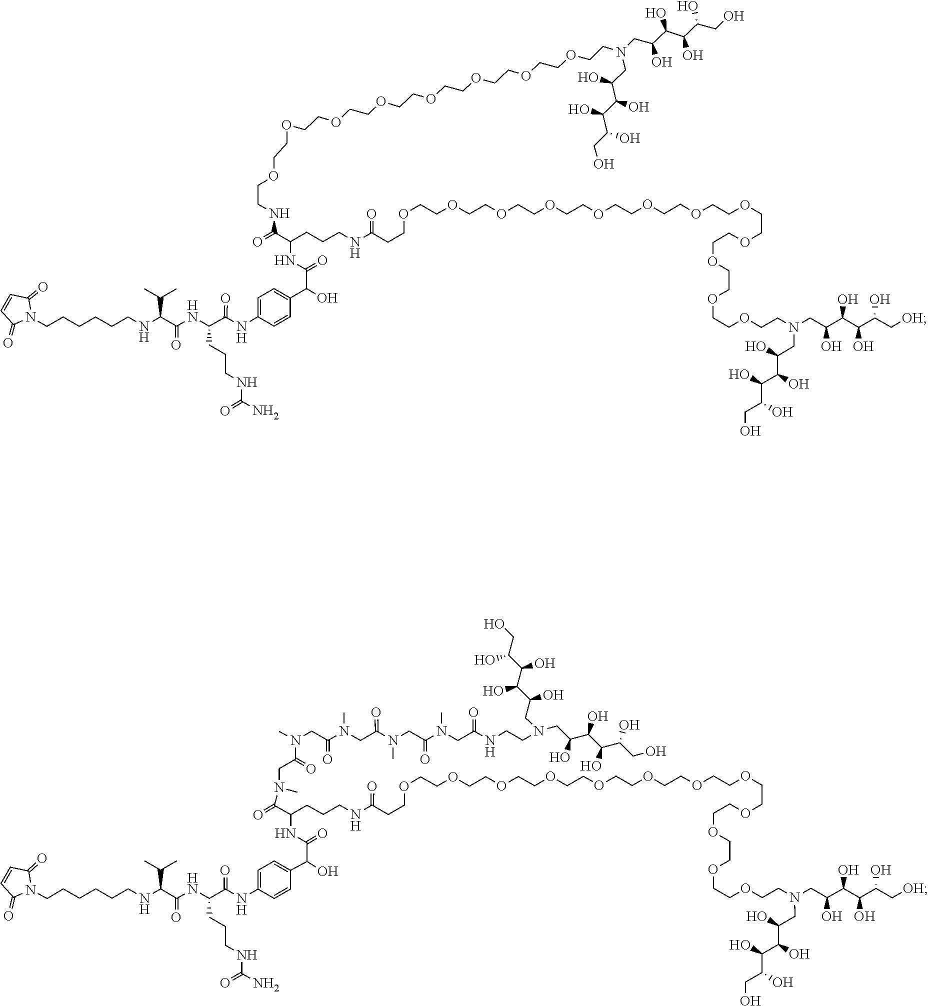 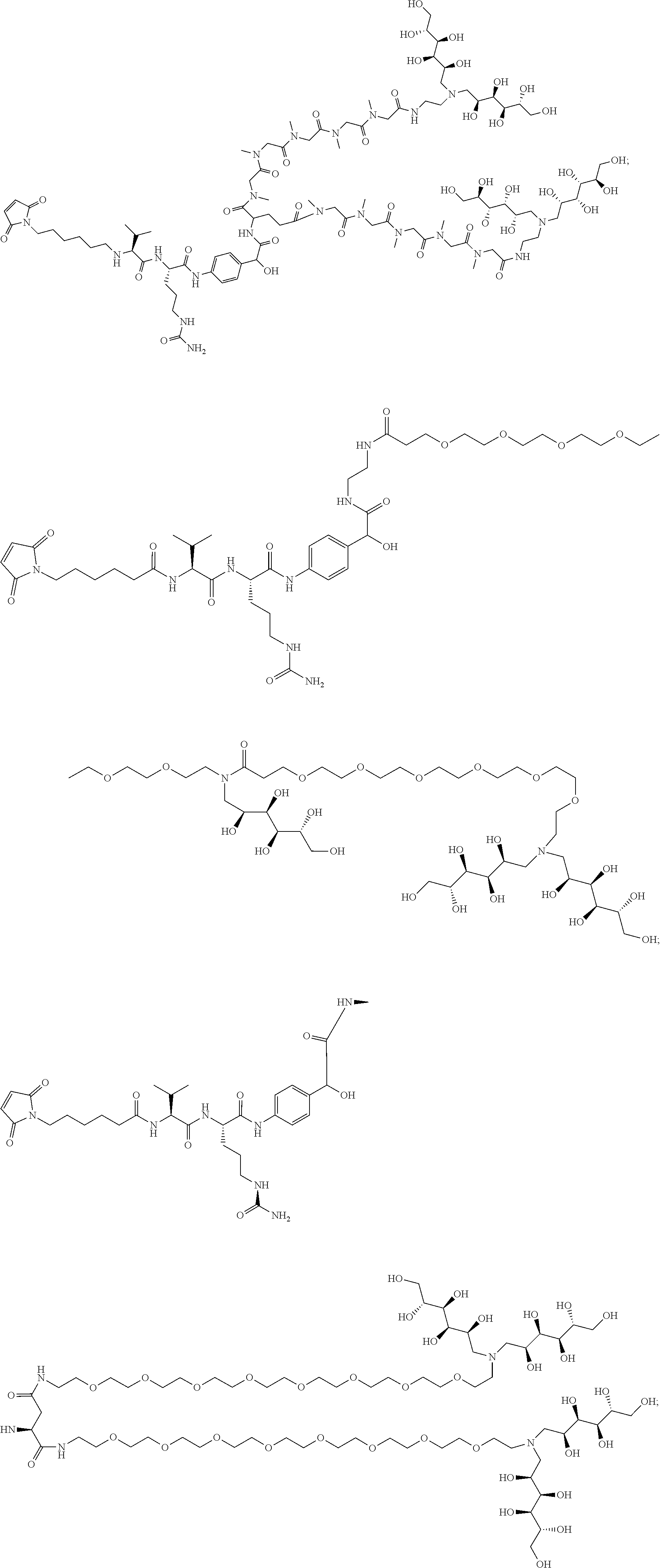 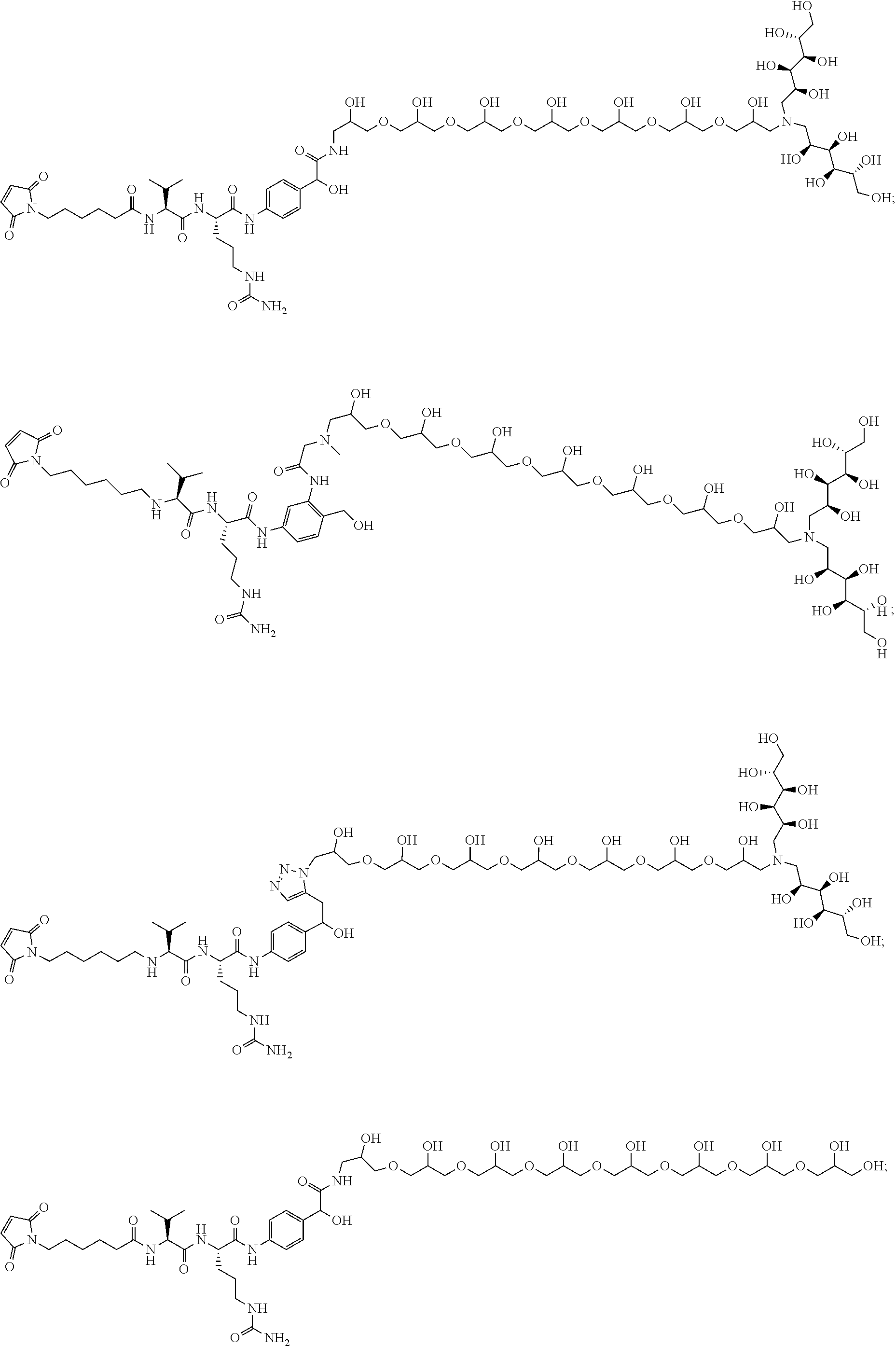 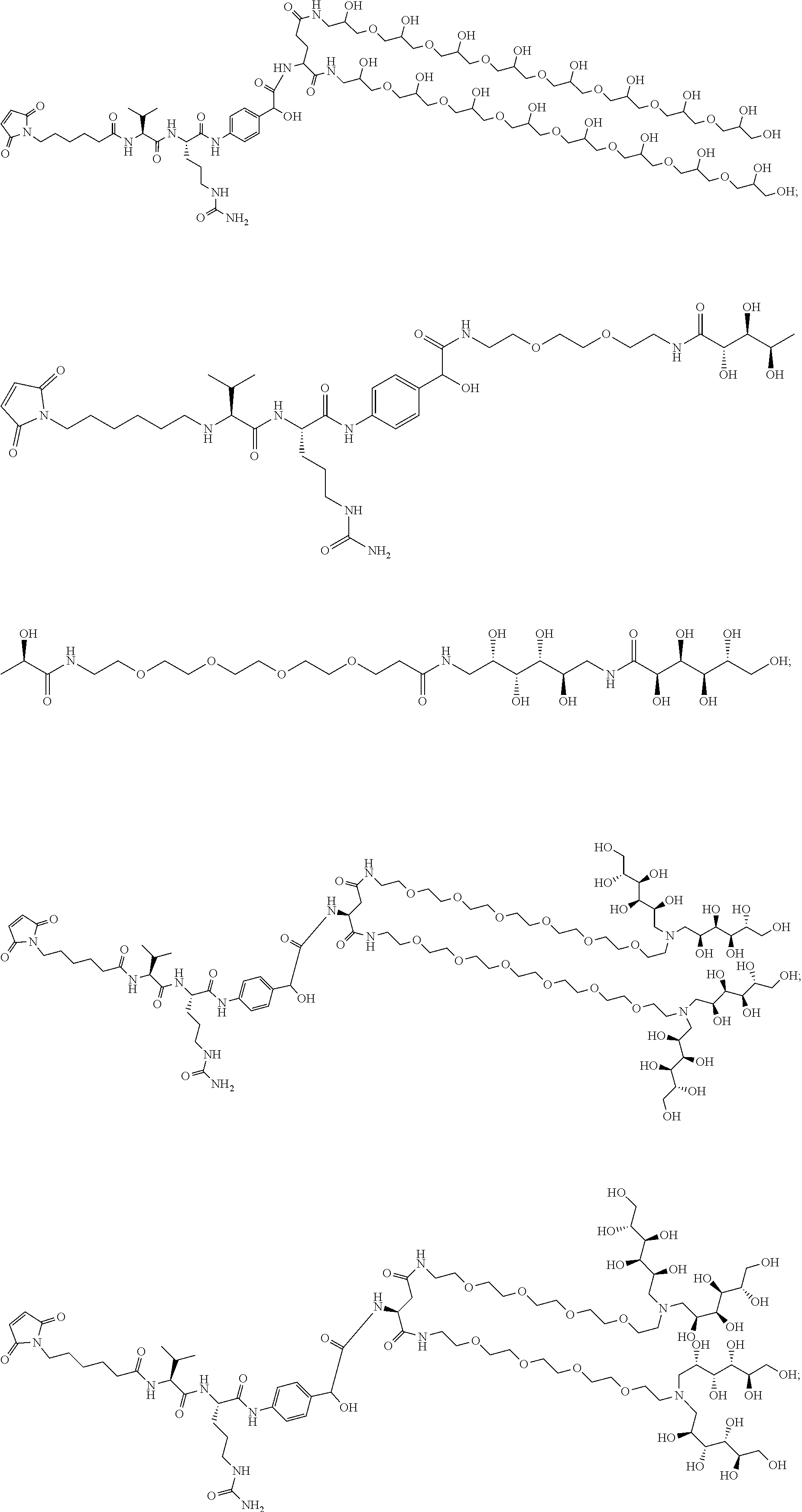 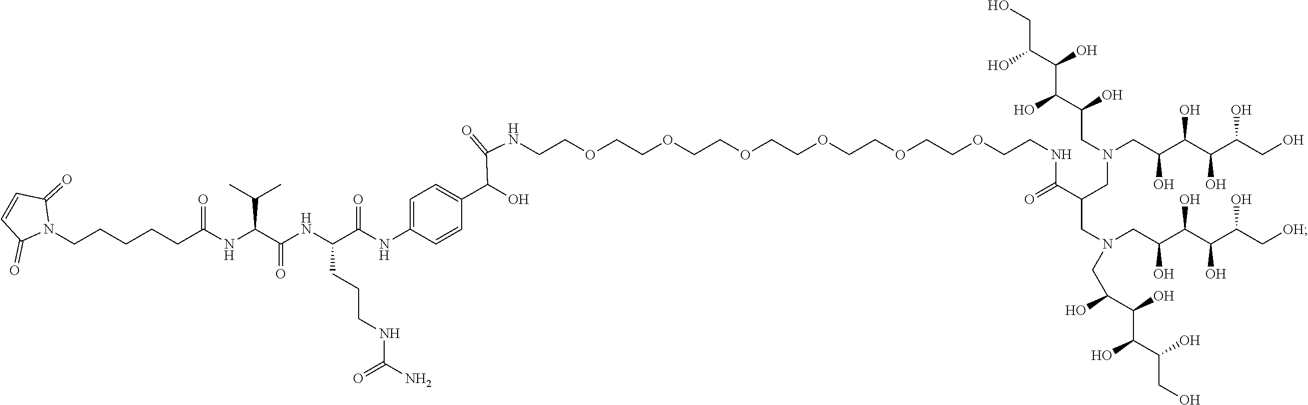 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)CN(C[C@H](O)[C@@H](O)[C@H](O)[C@H](O)CO)C[C@H](O)[C@@H](O)[C@H](O)[C@H](O)CO)cc1] 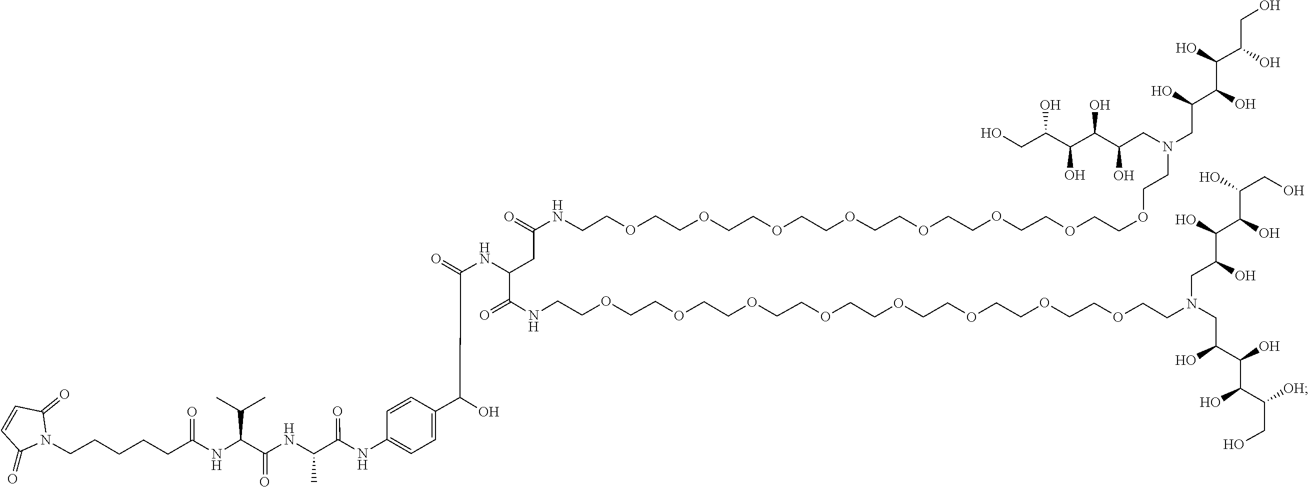 [SMILES: CC(C)[C@H](NC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 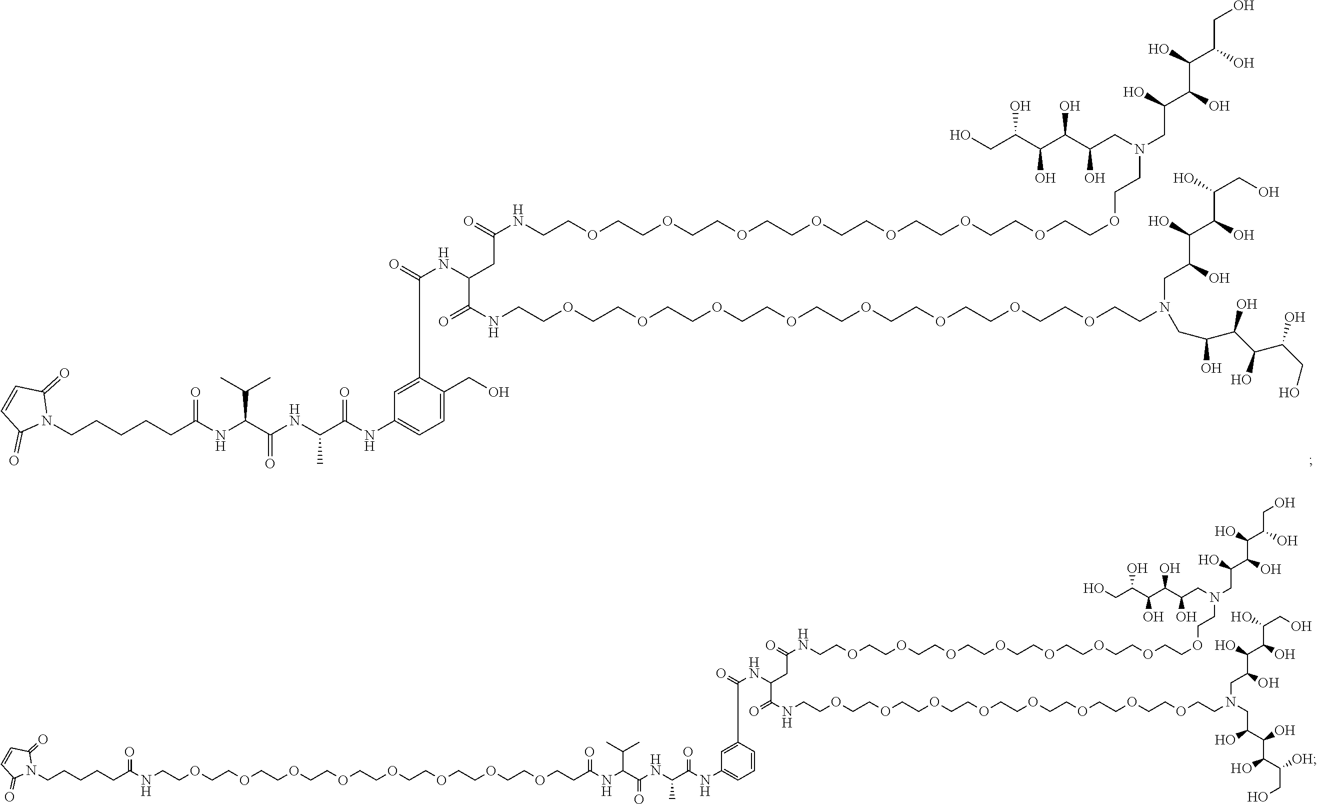 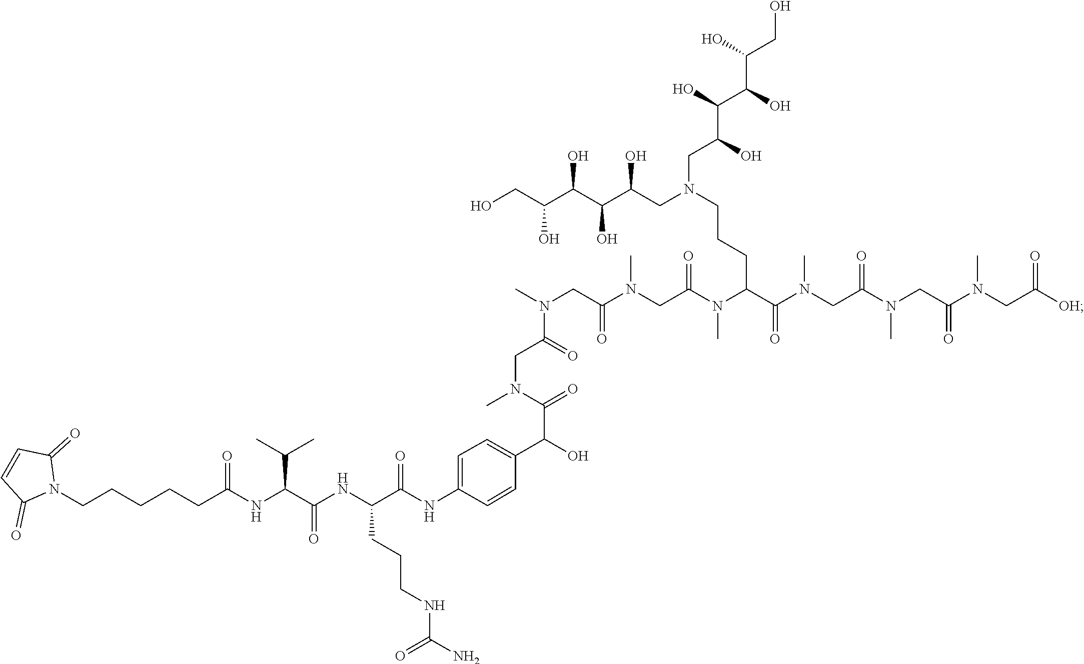 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 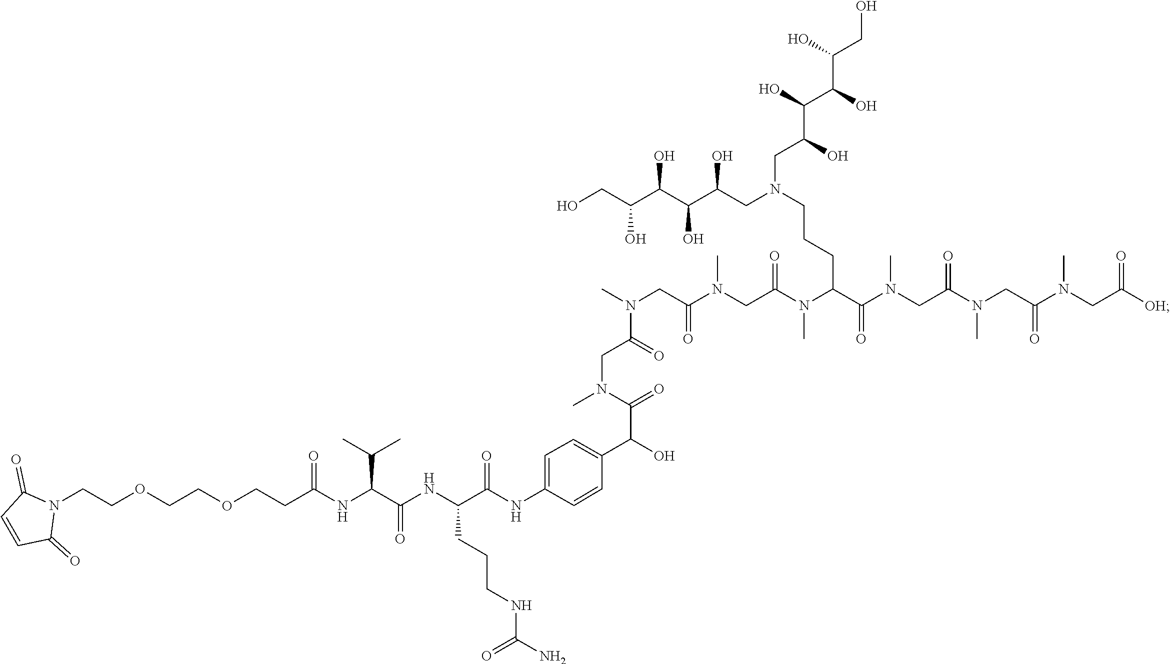 [SMILES: CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 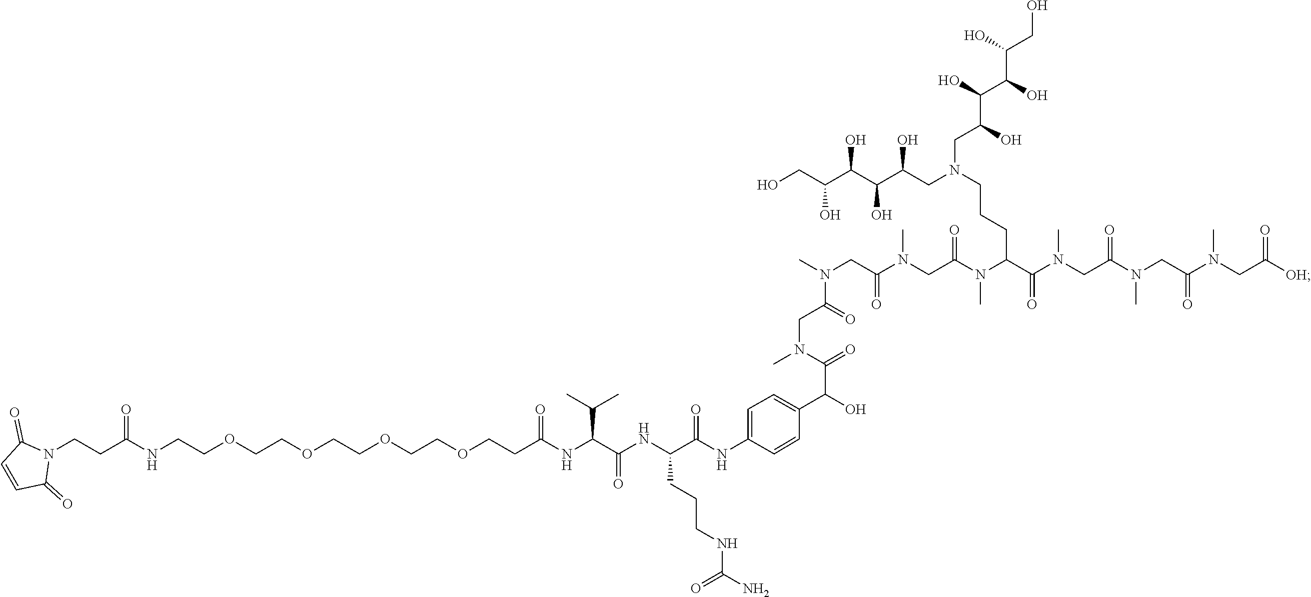 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 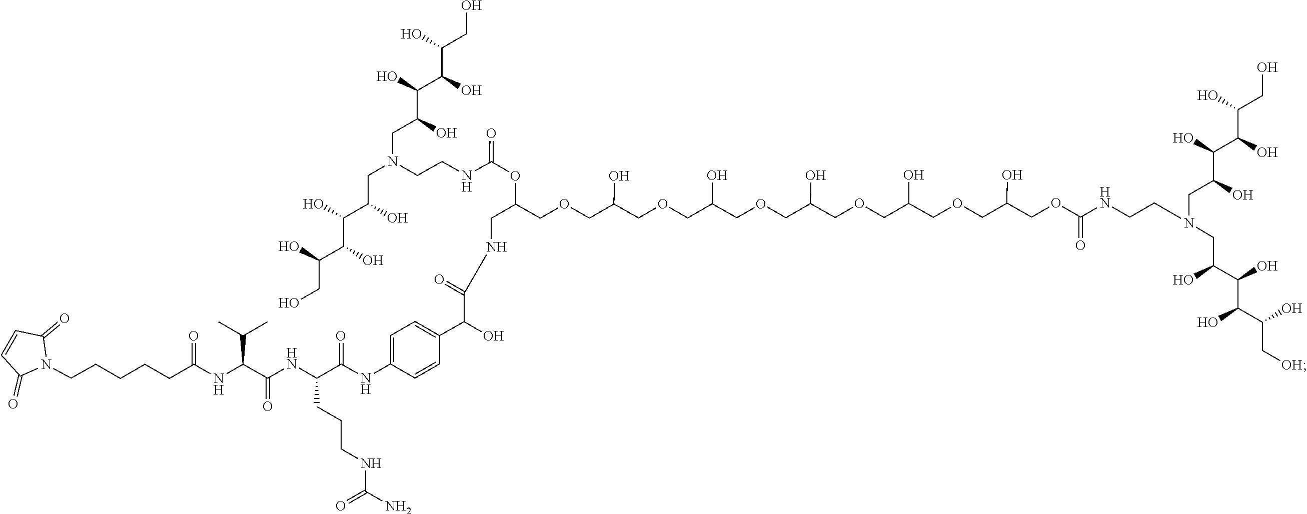 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1] 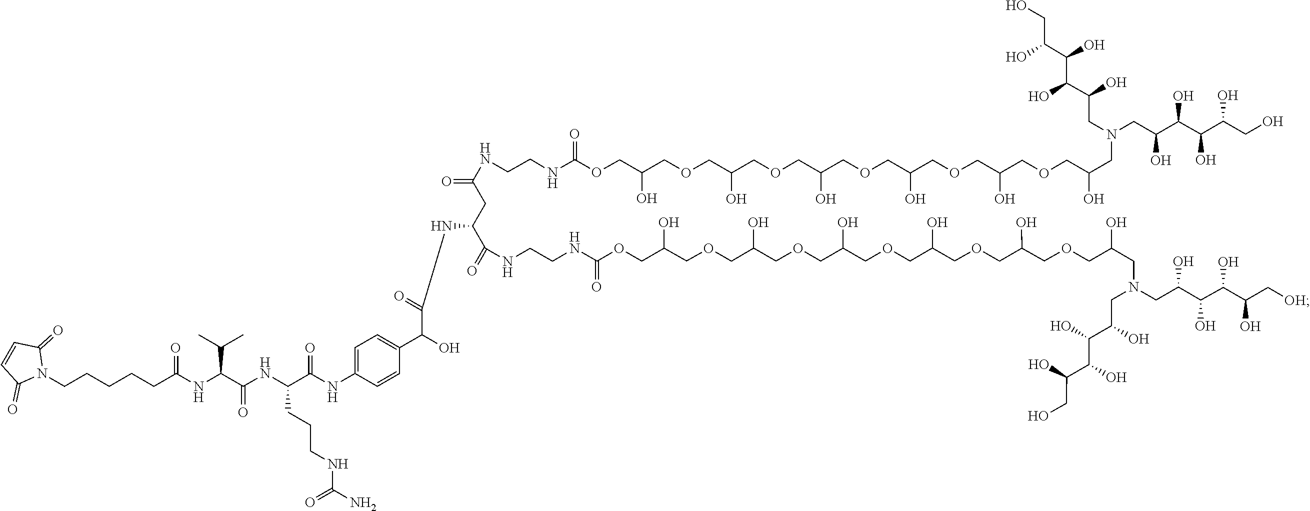 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 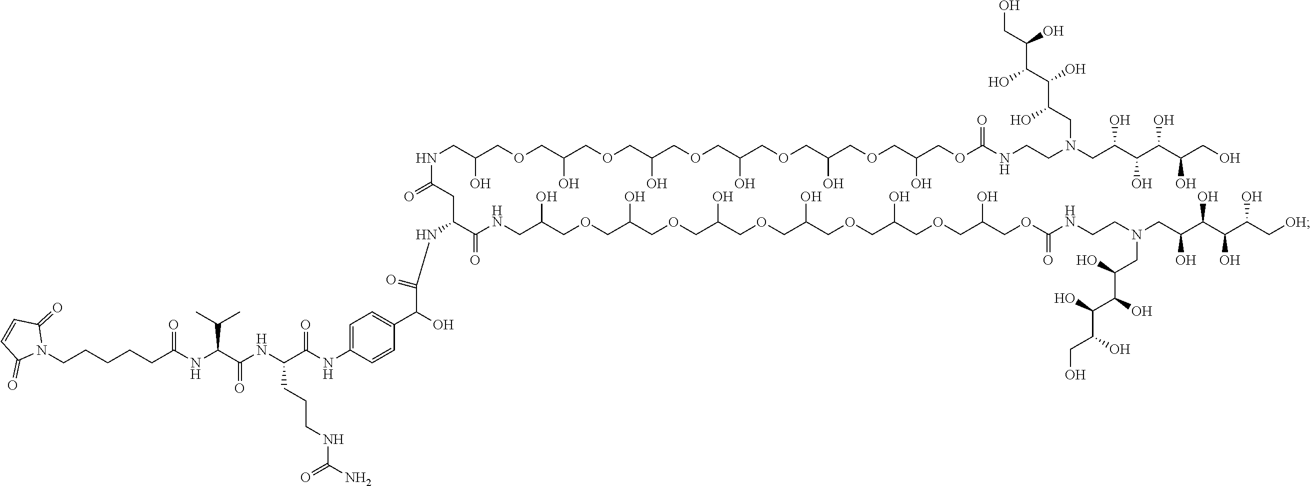 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)cc1] 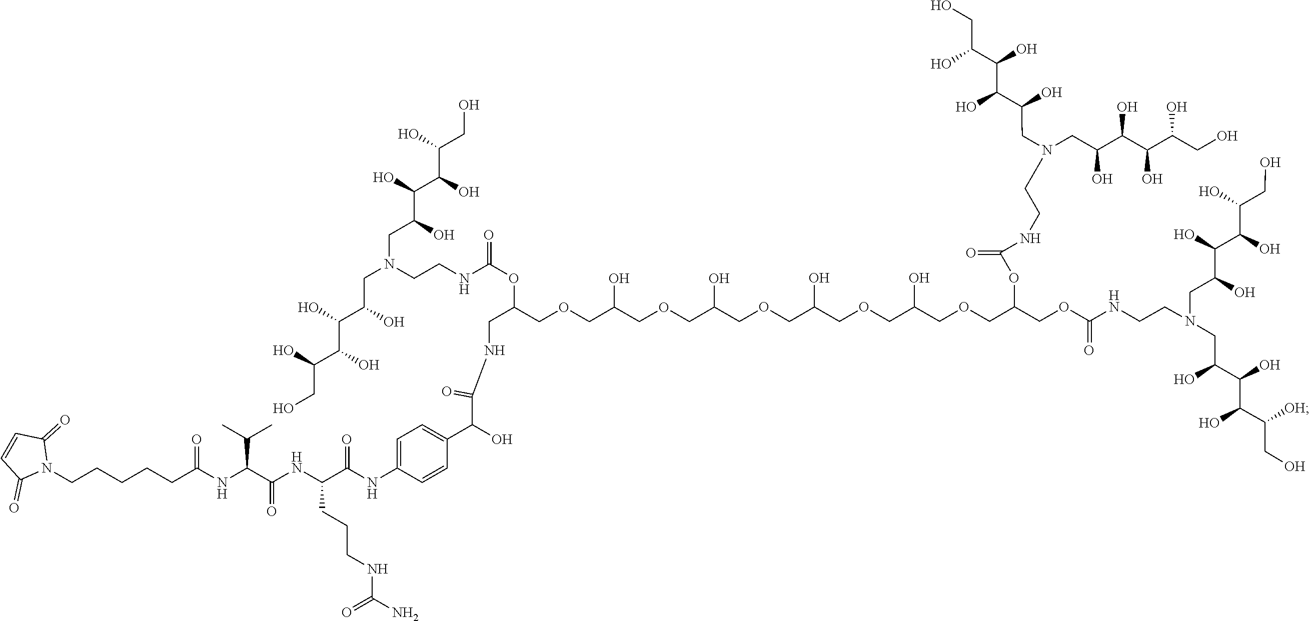 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1] 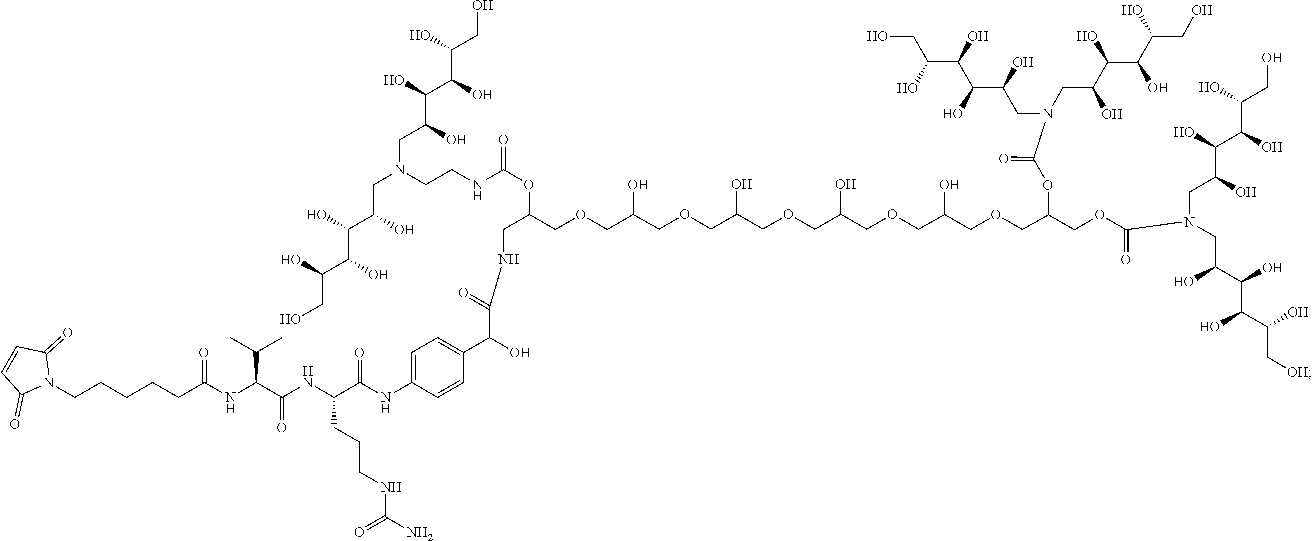 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(COC(=O)N(C[C@H](O)[C@@H](O)[C@H](O)[C@H](O)CO)C[C@H](O)[C@@H](O)[C@H](O)[C@H](O)CO)OC(=O)N(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1] 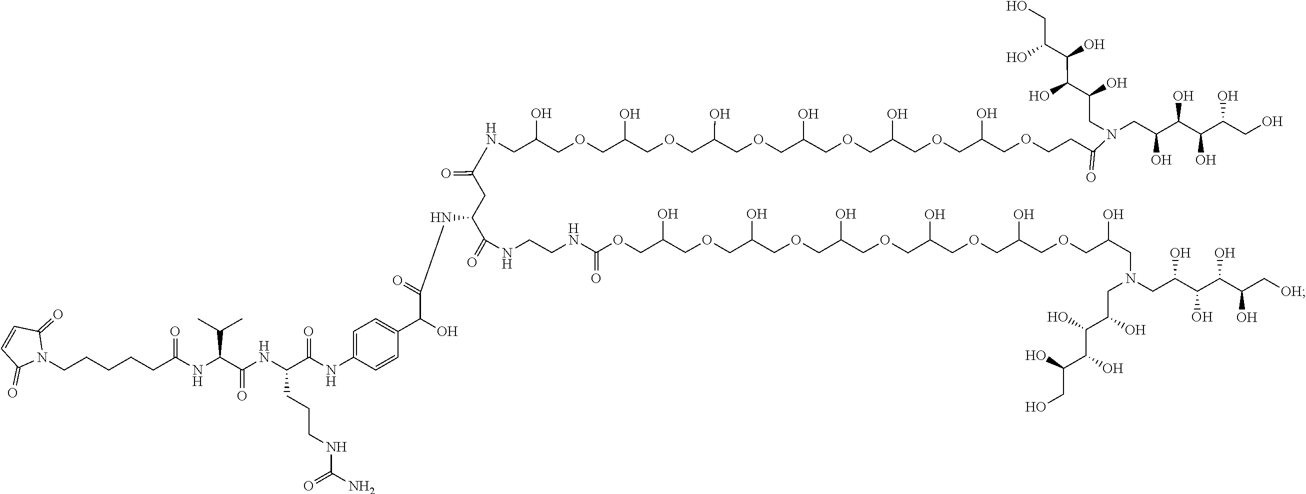 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COCCC(=O)N(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 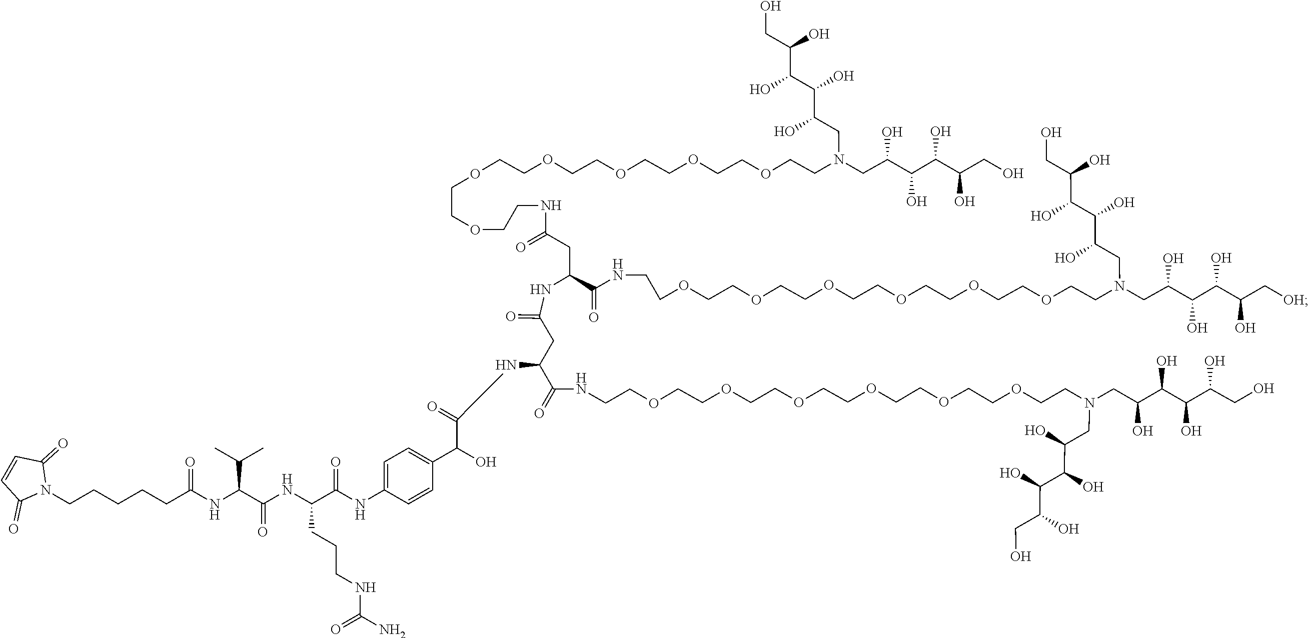 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 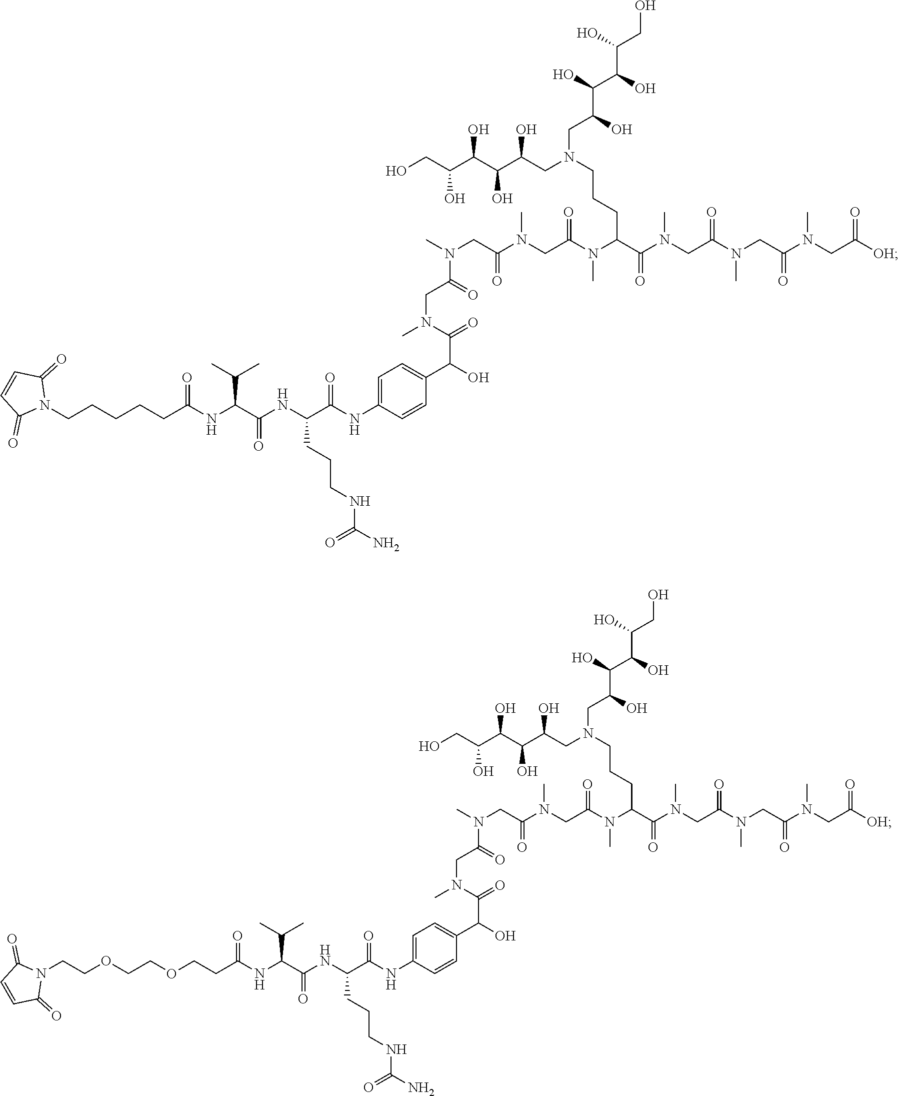 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1 ; CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 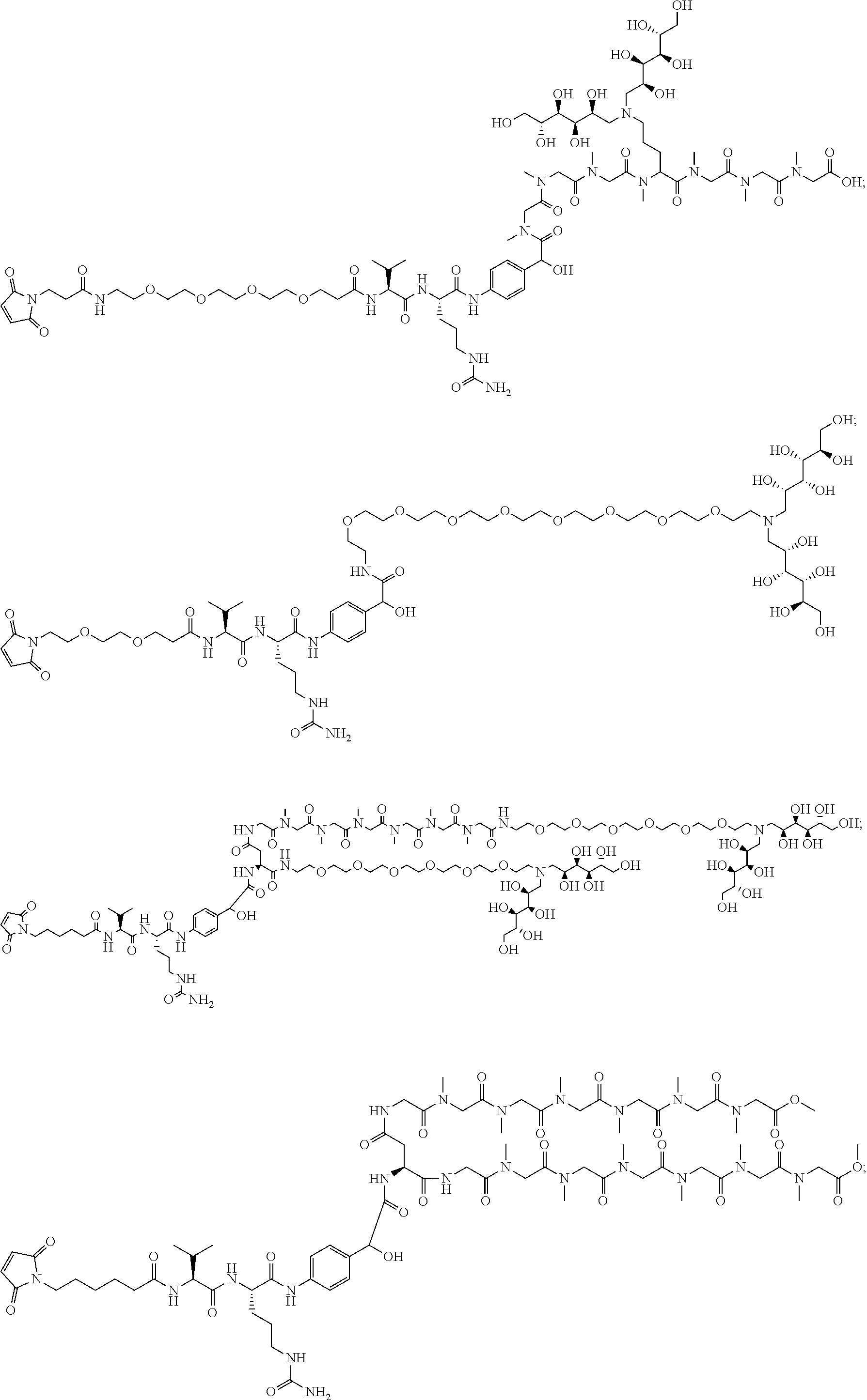 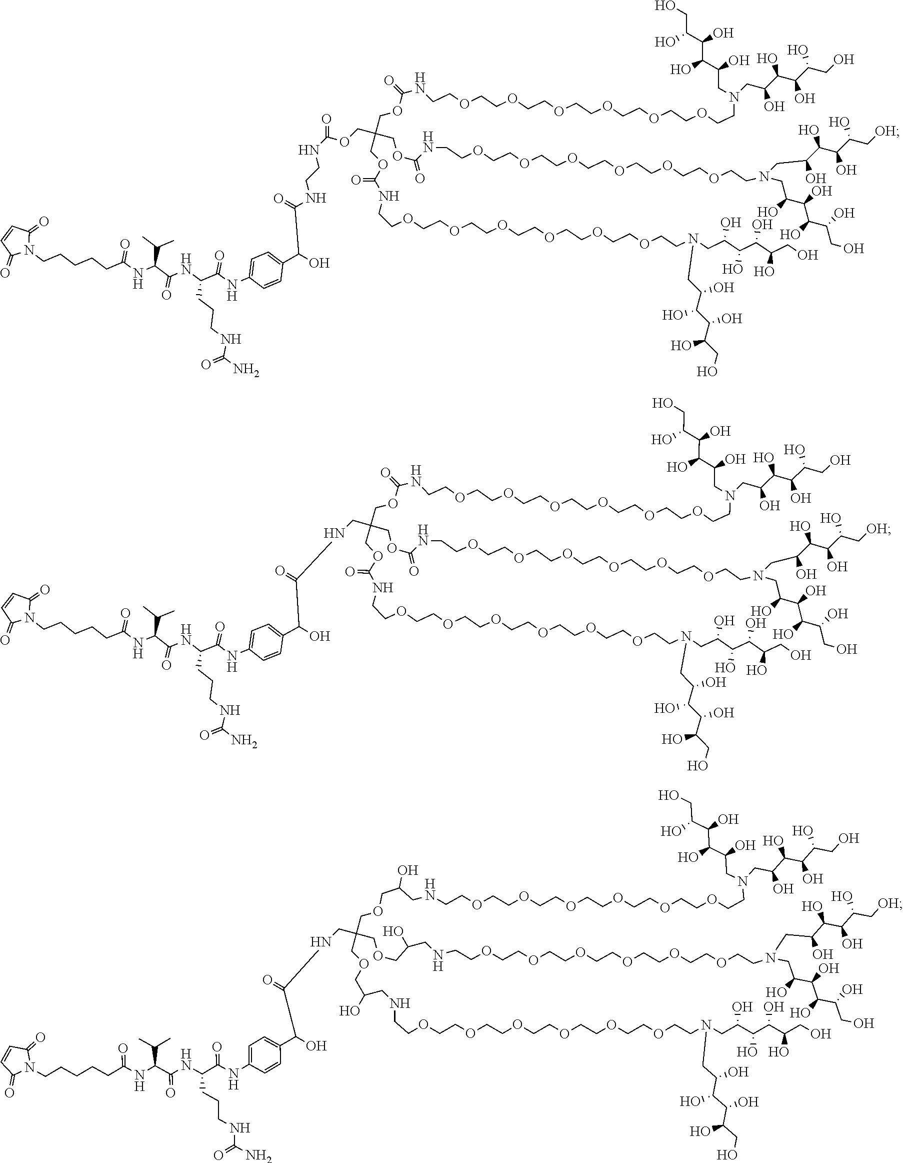 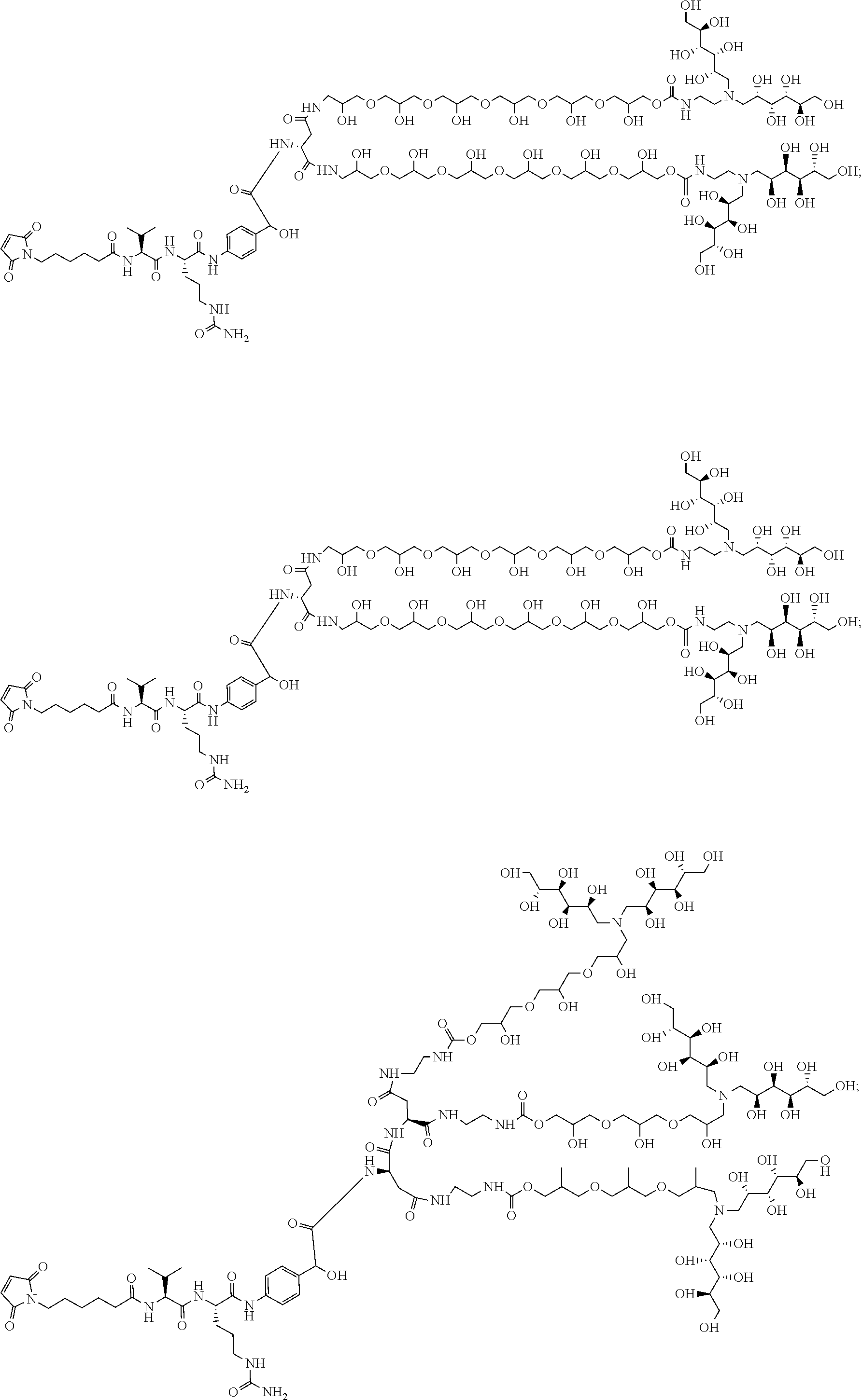 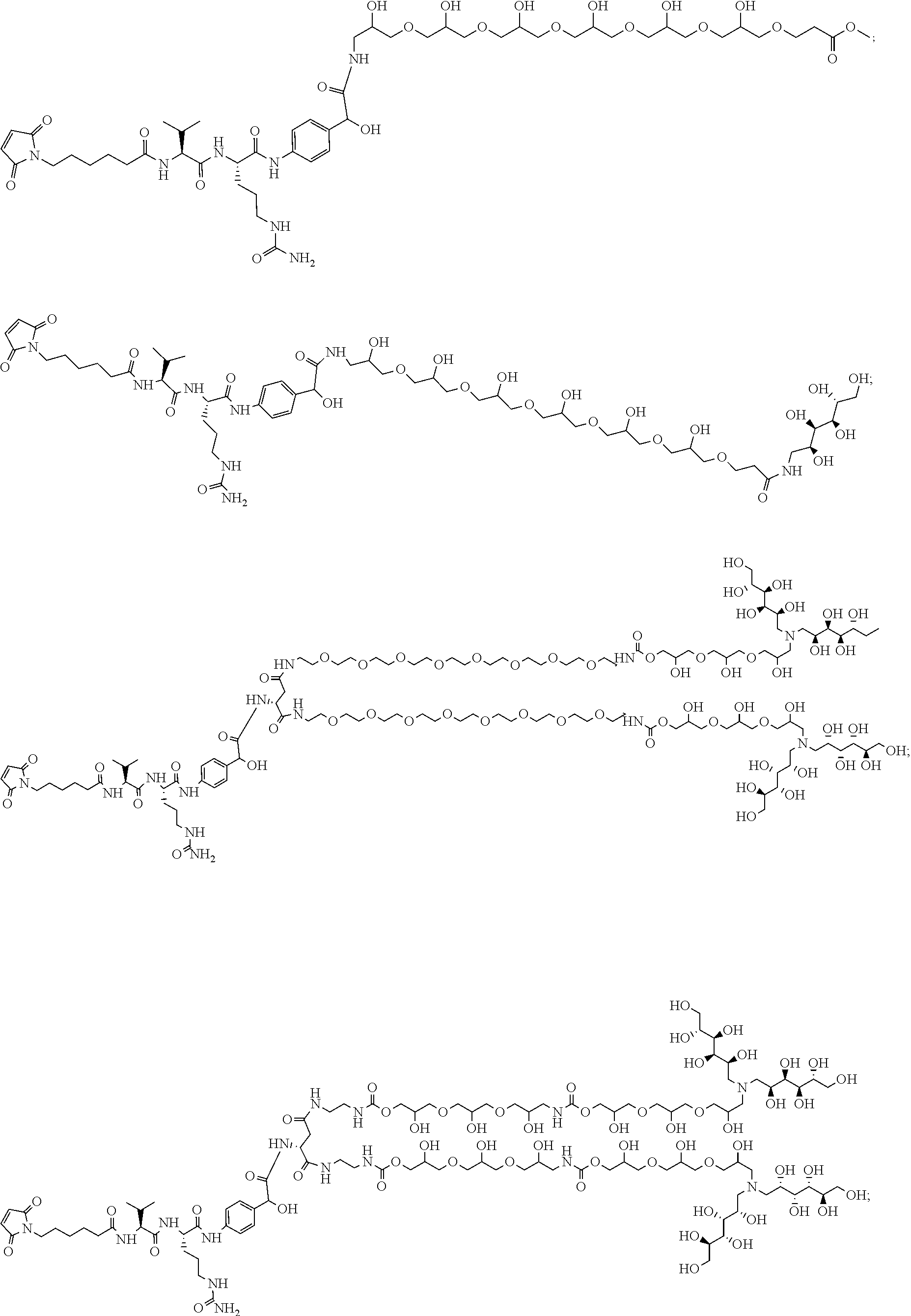 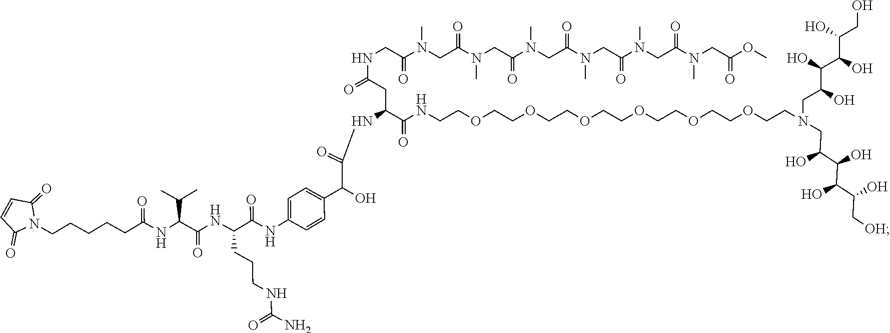 [SMILES: COC(=O)CN(C)C(=O)CN(C)C(=O)CN(C)C(=O)CN(C)C(=O)CN(C)C(=O)CN(C)C(=O)CNC(=O)C[C@H](NC(=O)C(O)c1ccc(NC(=O)[C@H](CCCNC(N)=O)NC(=O)[C@@H](NC(=O)CCCCCN2C(=O)C=CC2=O)C(C)C)cc1)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO] 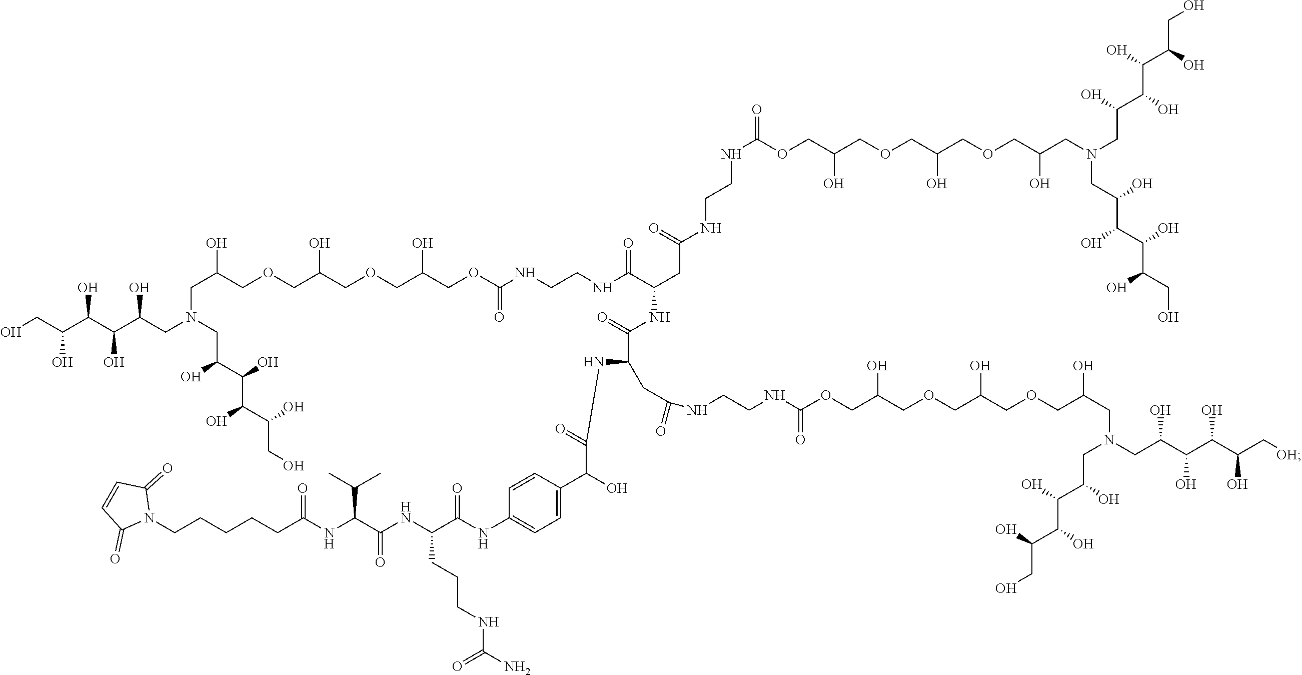 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 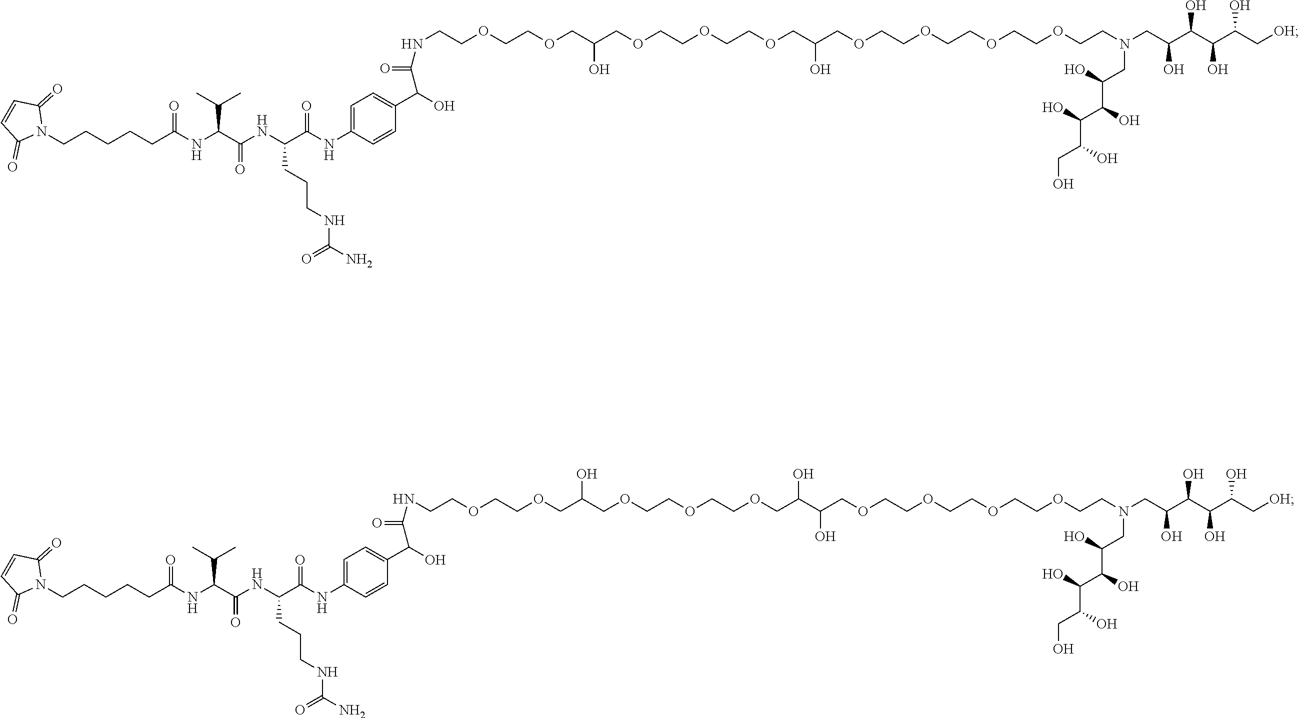 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)COCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)C(O)COCCOCCOCCOCCN(C[C@H](O)[C@@H](O)[C@H](O)[C@H](O)CO)C[C@H](O)[C@@H](O)[C@H](O)[C@H](O)CO)cc1] 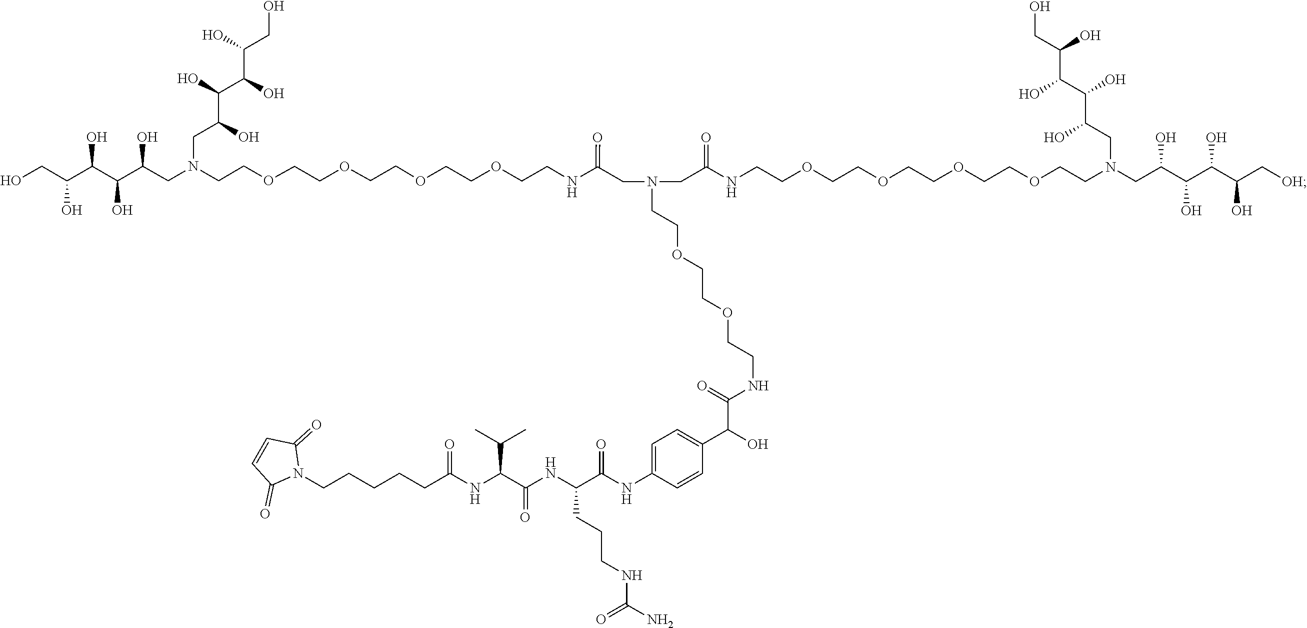 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCN(CC(=O)NCCOCCOCCOCCOCCN(C[C@H](O)[C@@H](O)[C@H](O)[C@H](O)CO)C[C@H](O)[C@@H](O)[C@H](O)[C@H](O)CO)CC(=O)NCCOCCOCCOCCOCCN(C[C@H](O)[C@@H](O)[C@H](O)[C@H](O)CO)C[C@H](O)[C@@H](O)[C@H](O)[C@H](O)CO)cc1] 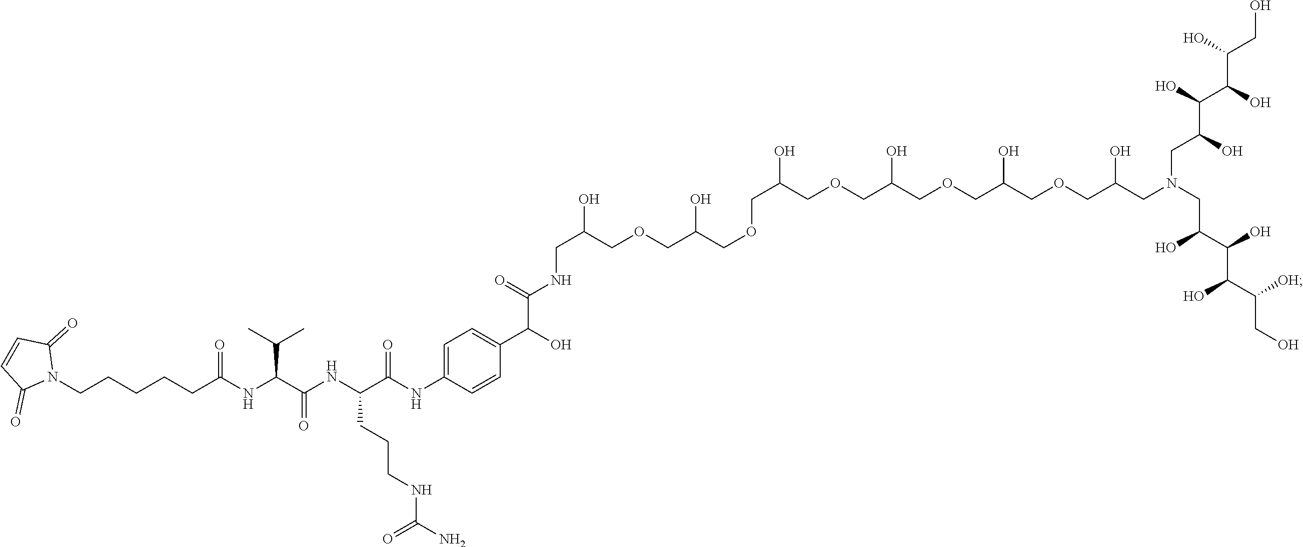 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 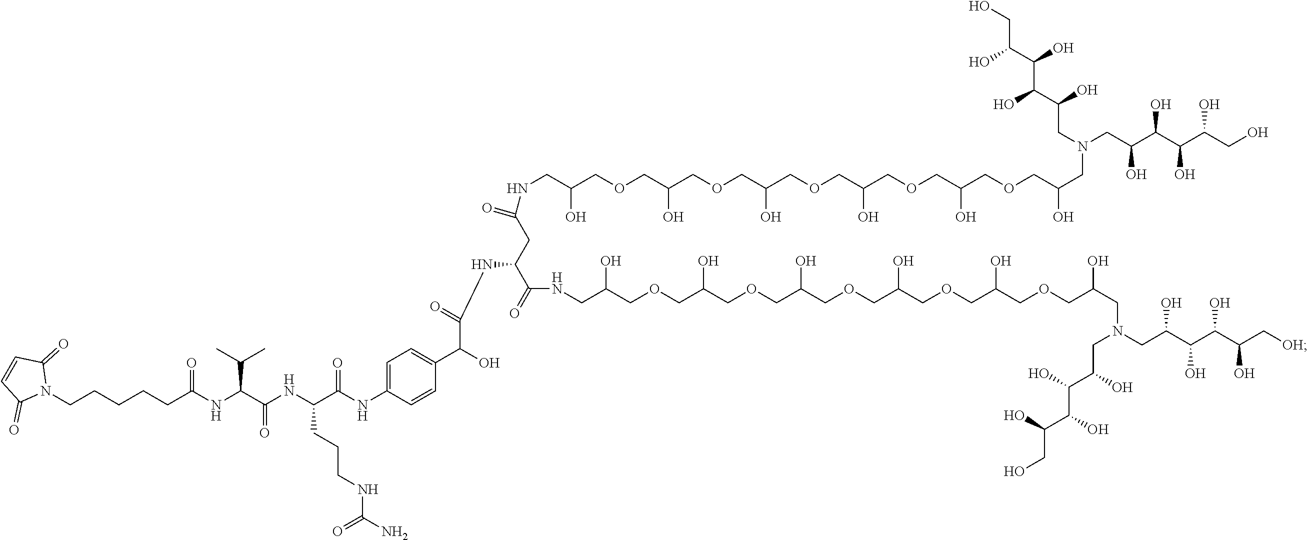 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 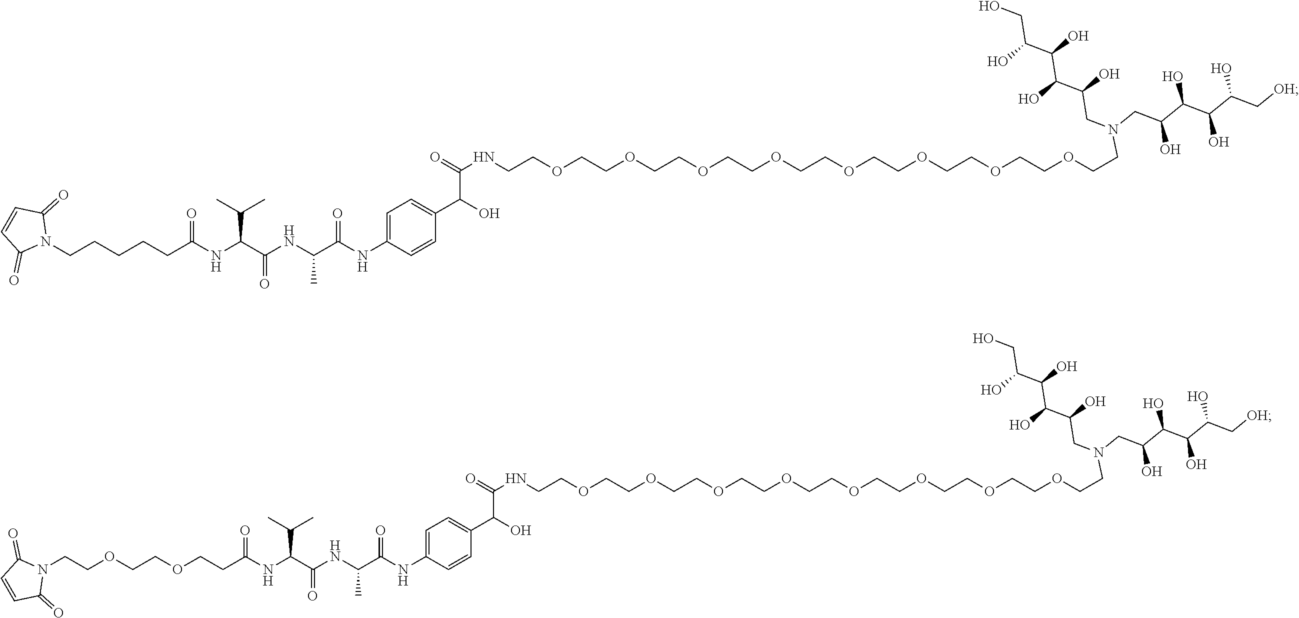 [SMILES: CC(C)[C@H](NC(=O)CCCCCN1C(=O)C=CC1=O)C(=O)N[C@@H](C)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCOCCOCCN1C(=O)C=CC1=O)C(=O)N[C@@H](C)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 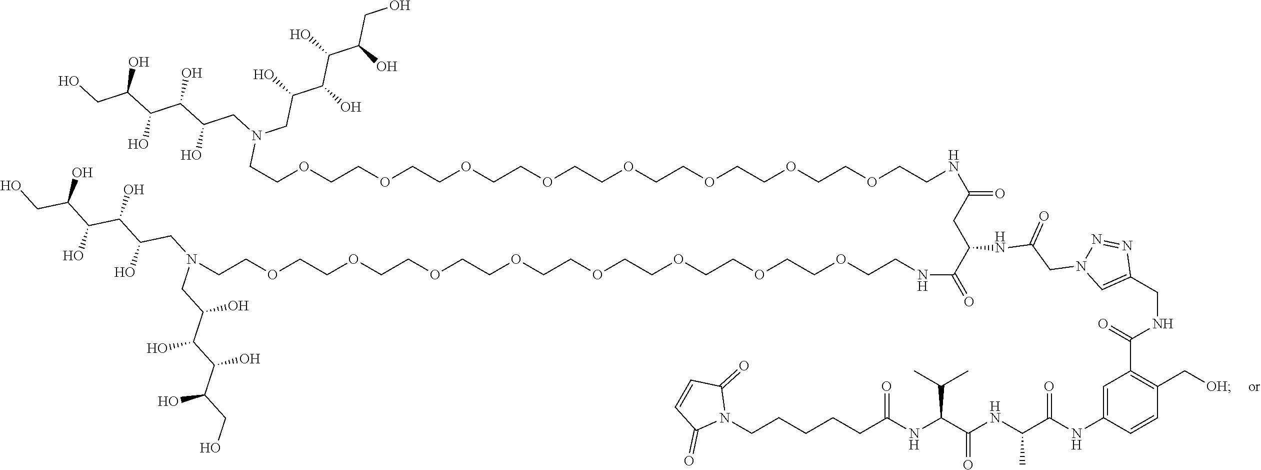 [SMILES: CC(C)[C@H](NC(=O)CCCCCN1C(=O)C=CC1=O)C(=O)N[C@@H](C)C(=O)Nc1ccc(CO)c(C(=O)NCc2cn(CC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)nn2)c1 ; "or"] 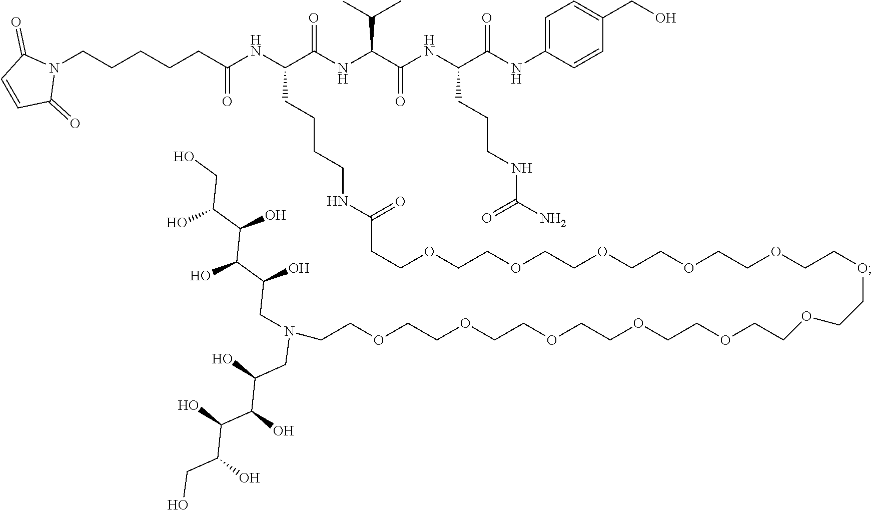 [SMILES: CC(C)[C@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN1C(=O)C=CC1=O)C(=O)N[C@@H](CCCNC(N)=O)C(=O)Nc1ccc(CO)cc1]
wherein H on the benzylic OH is optionally replaced with a bond to the drug unit or to a linking group attached to the drug unit.

51. The conjugate of claim 24, wherein the linker compound has one of the following structures: 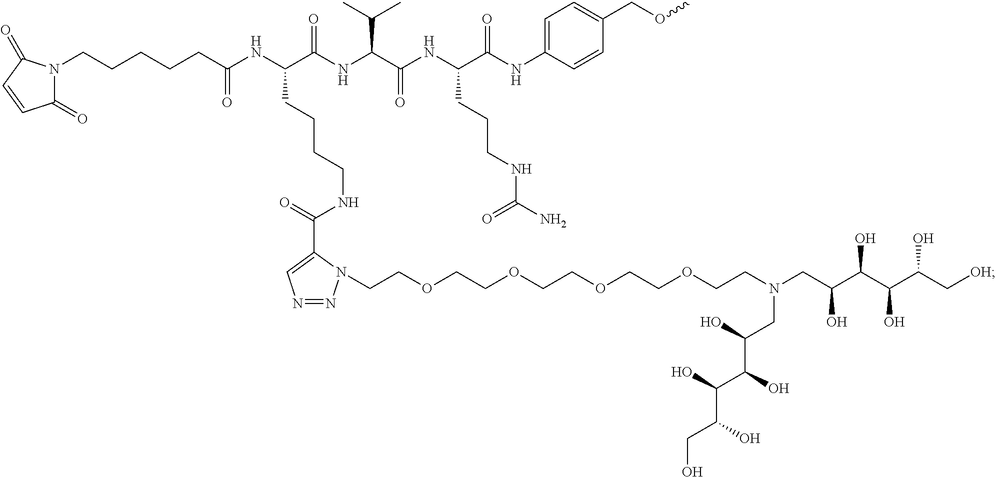 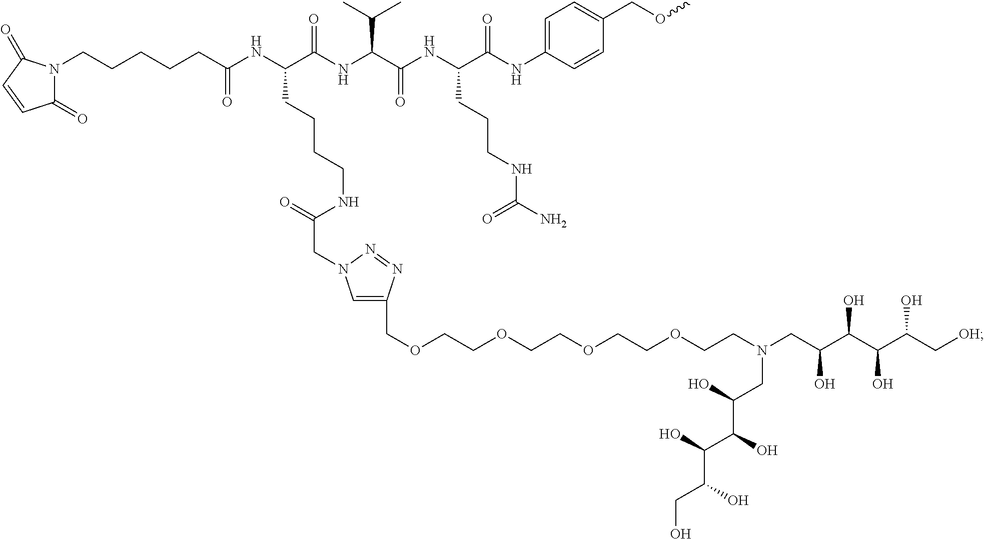 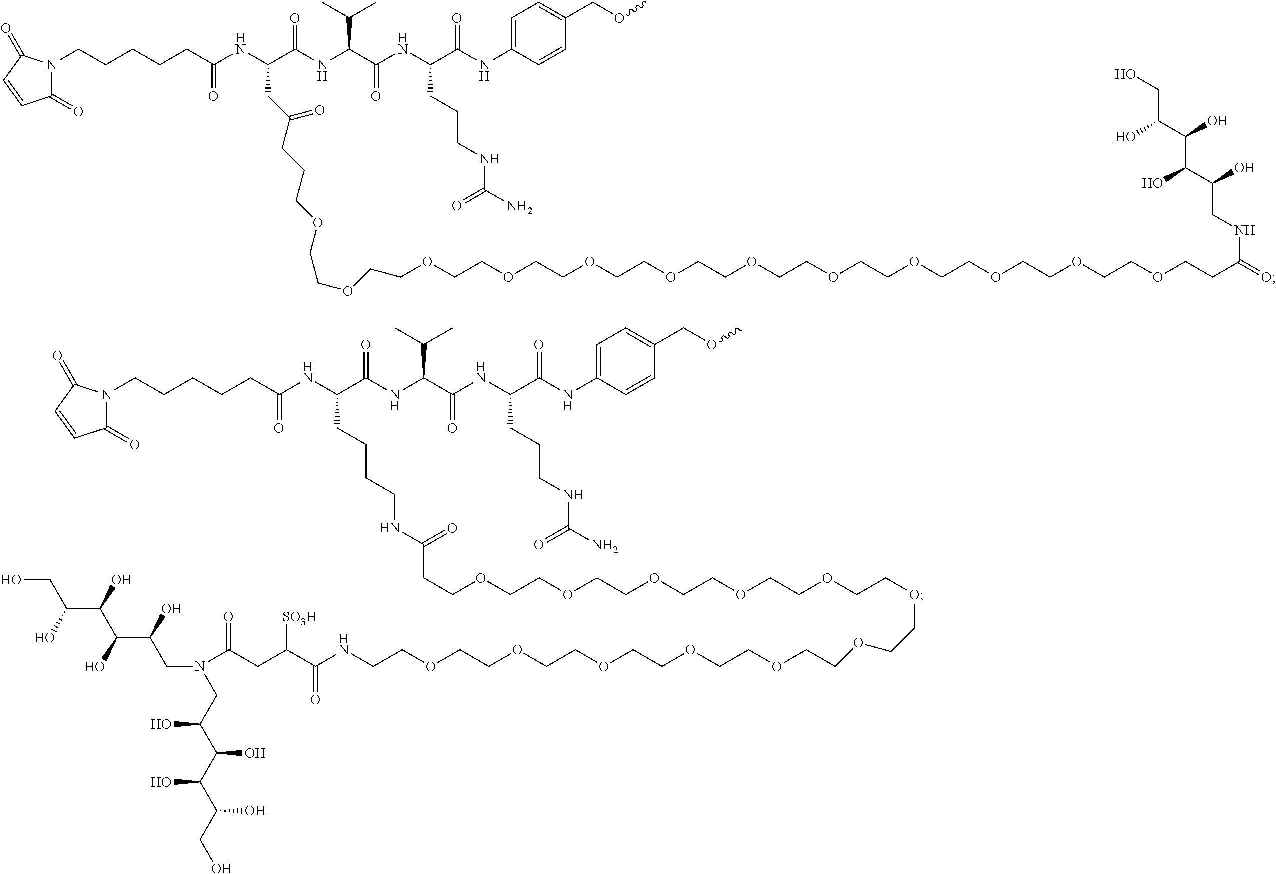 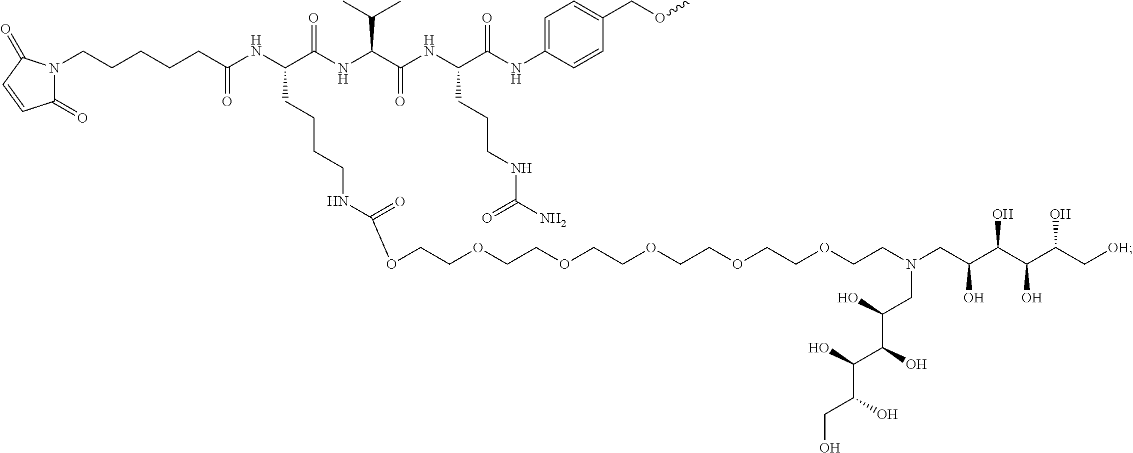 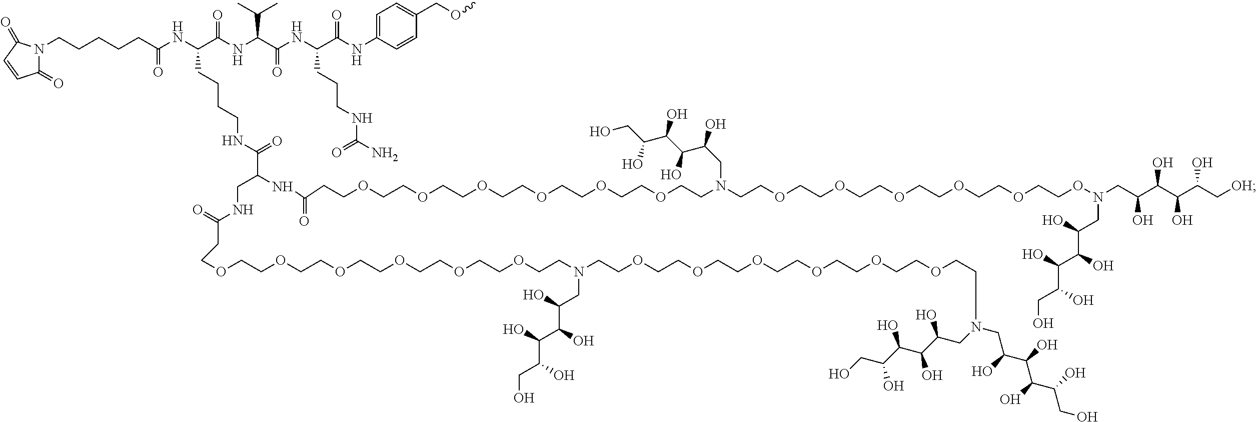 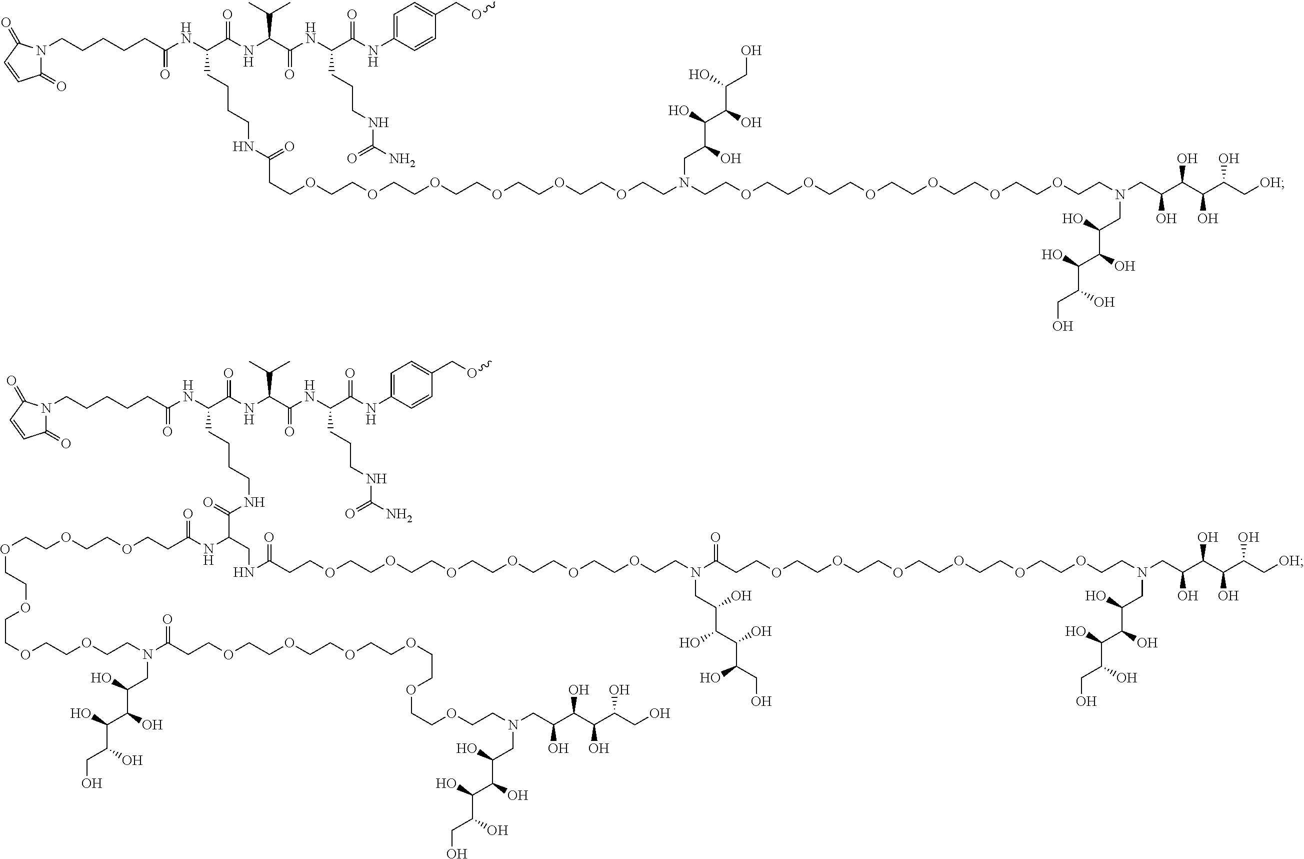 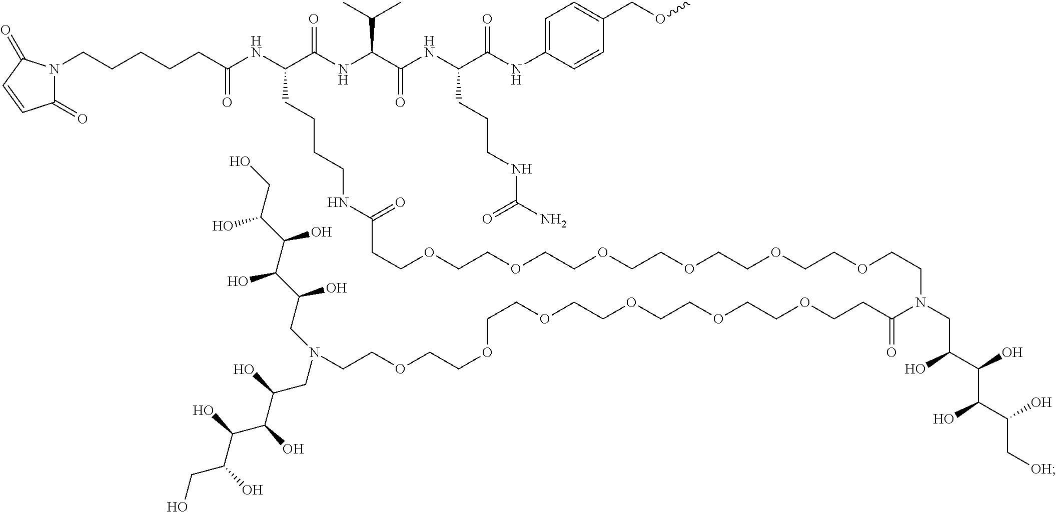 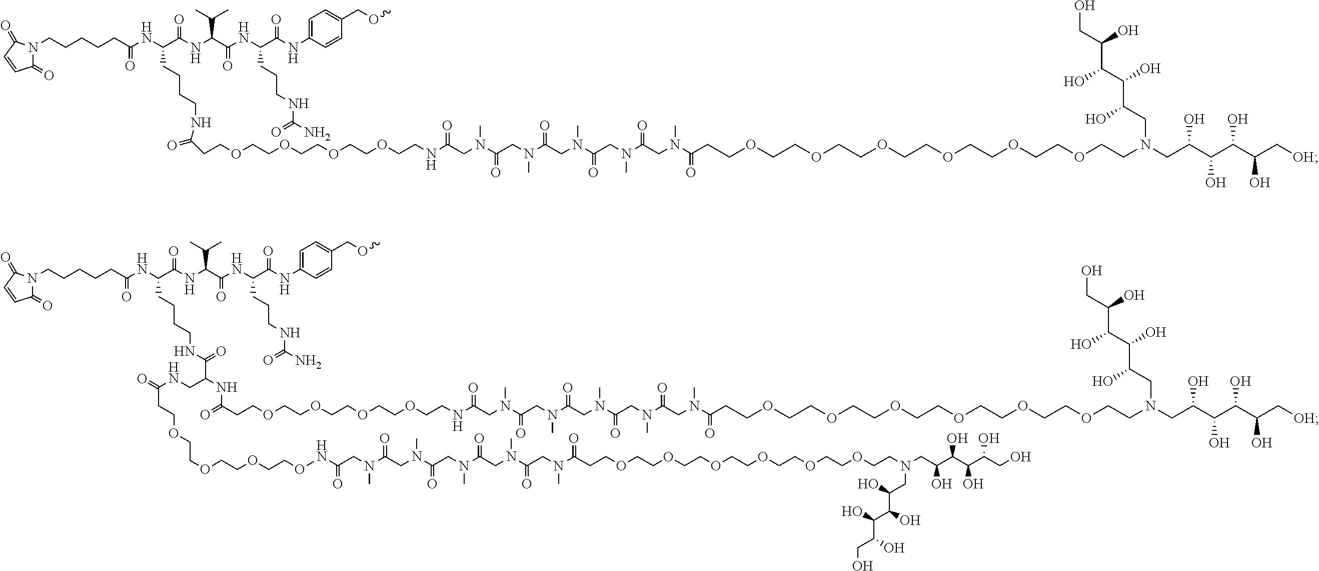 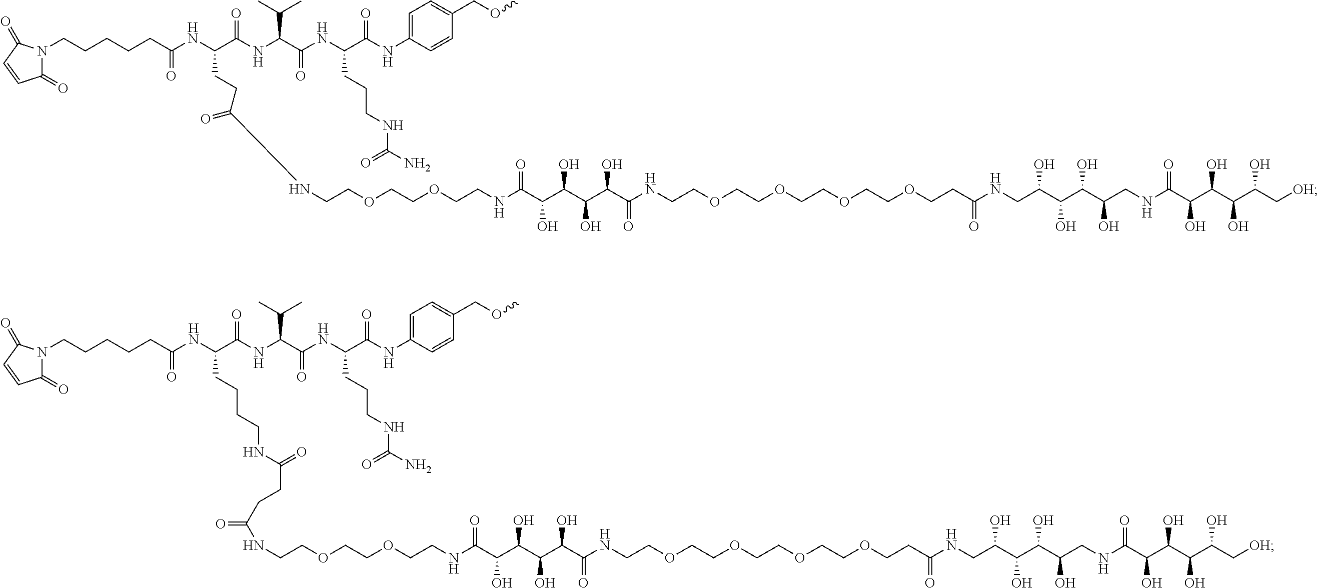 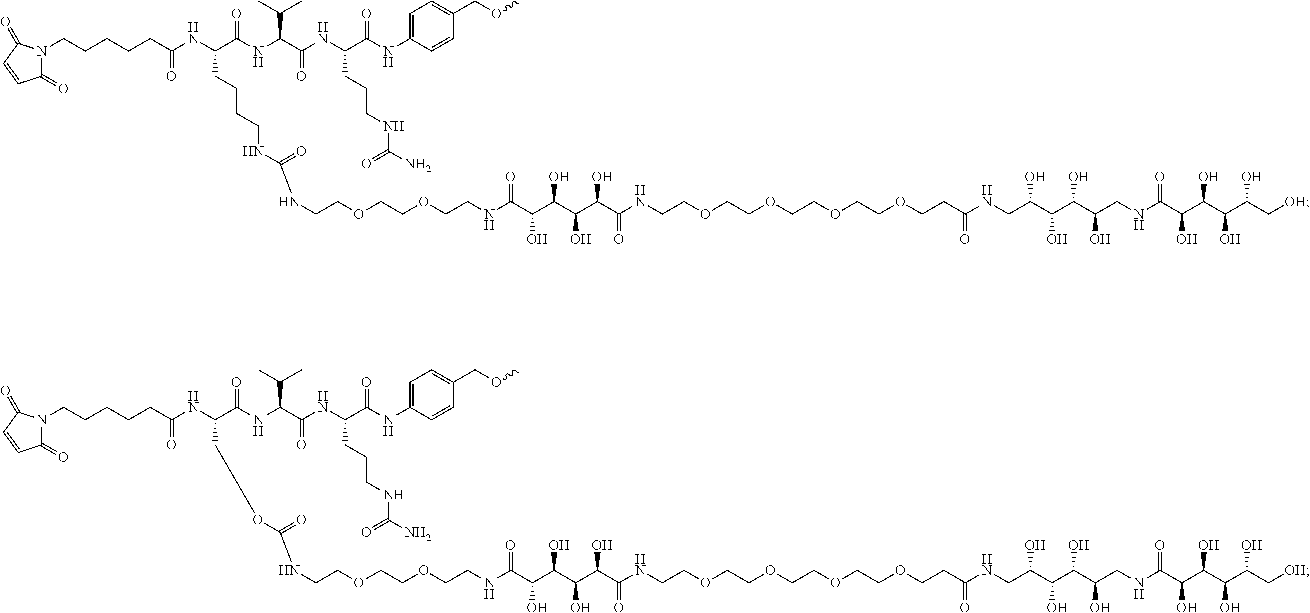 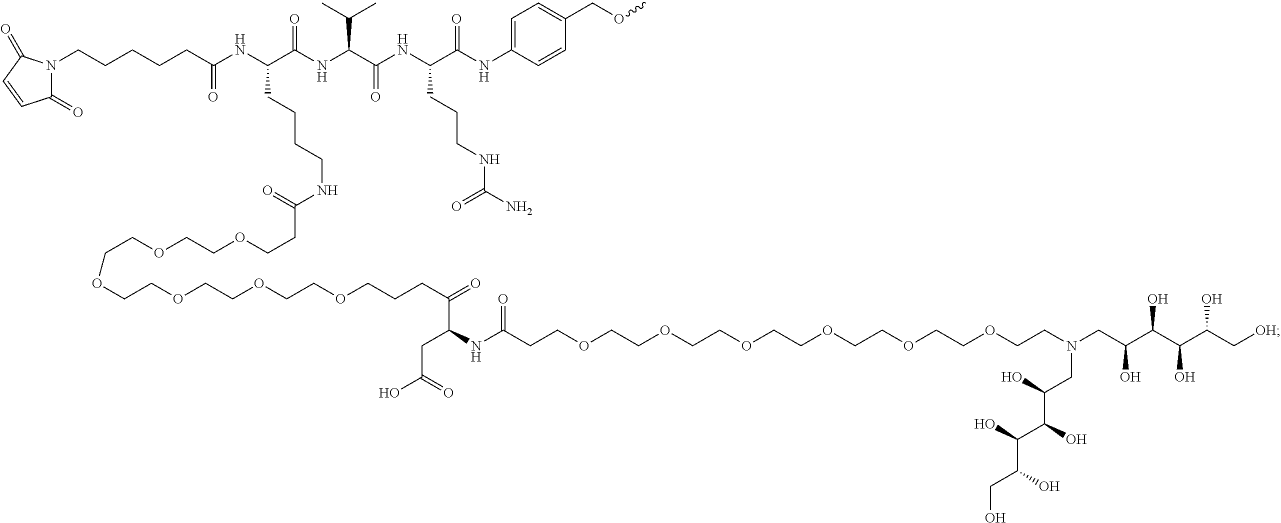 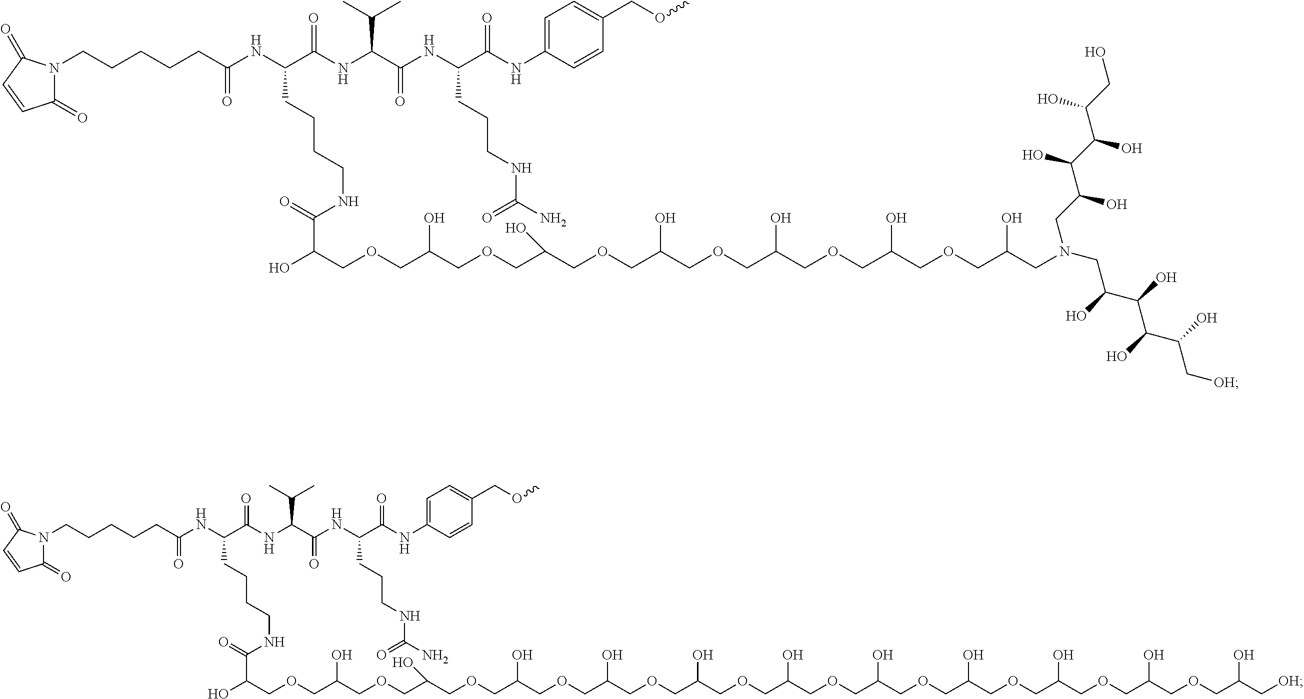 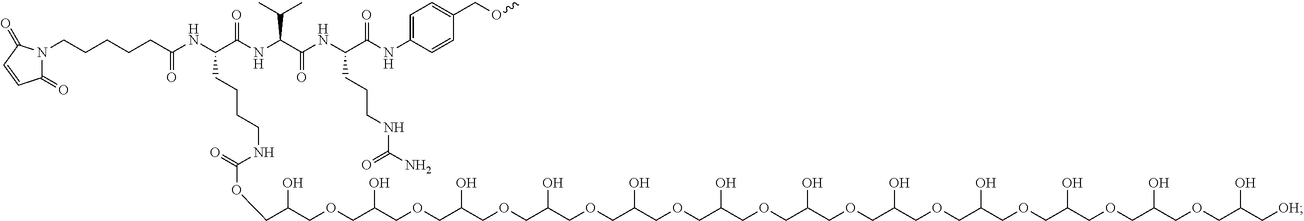 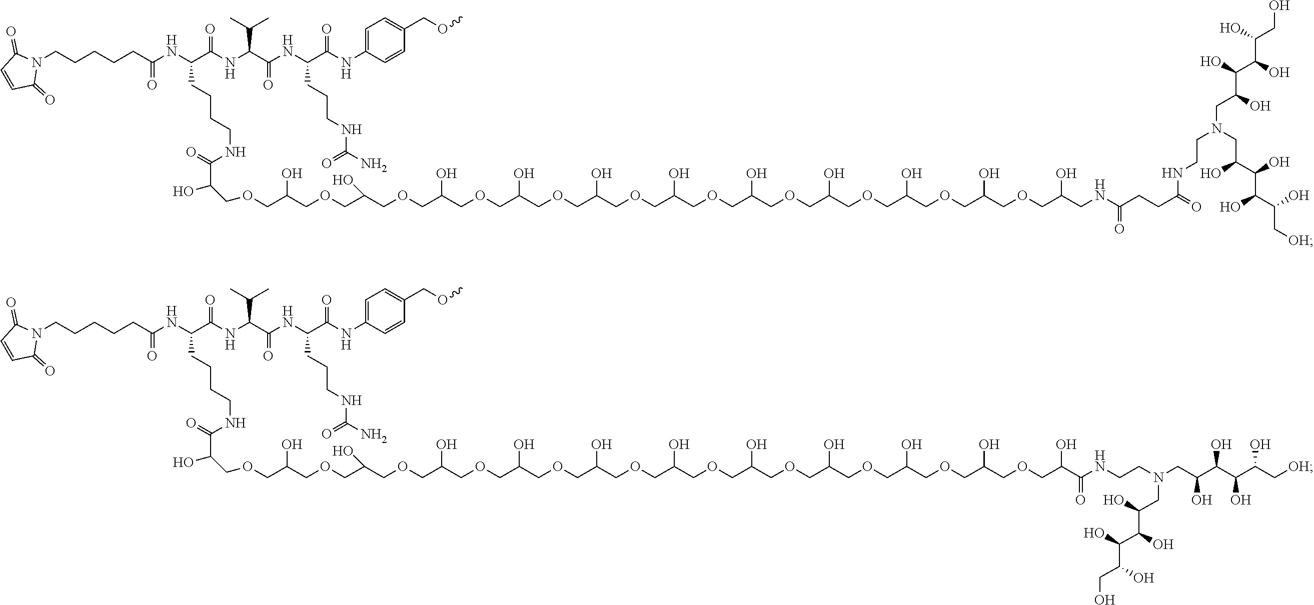 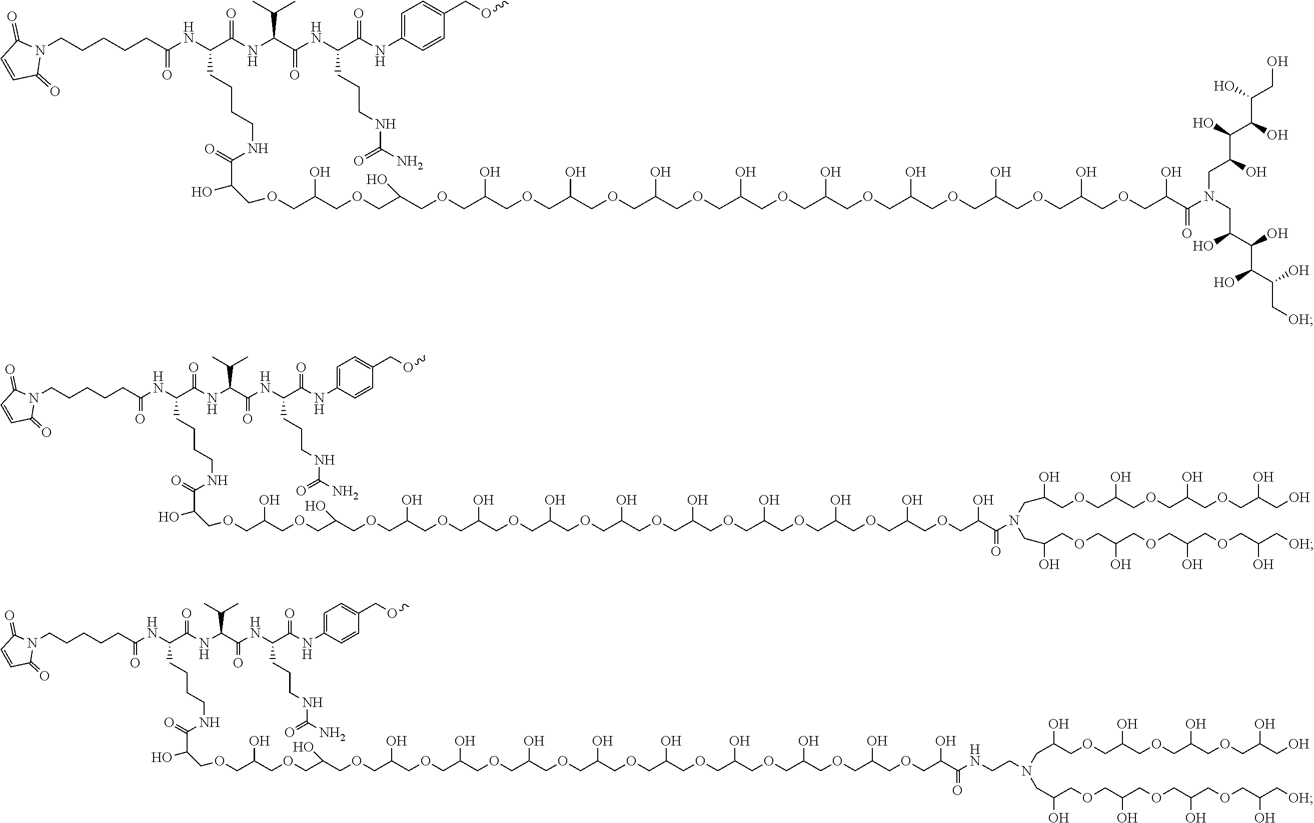 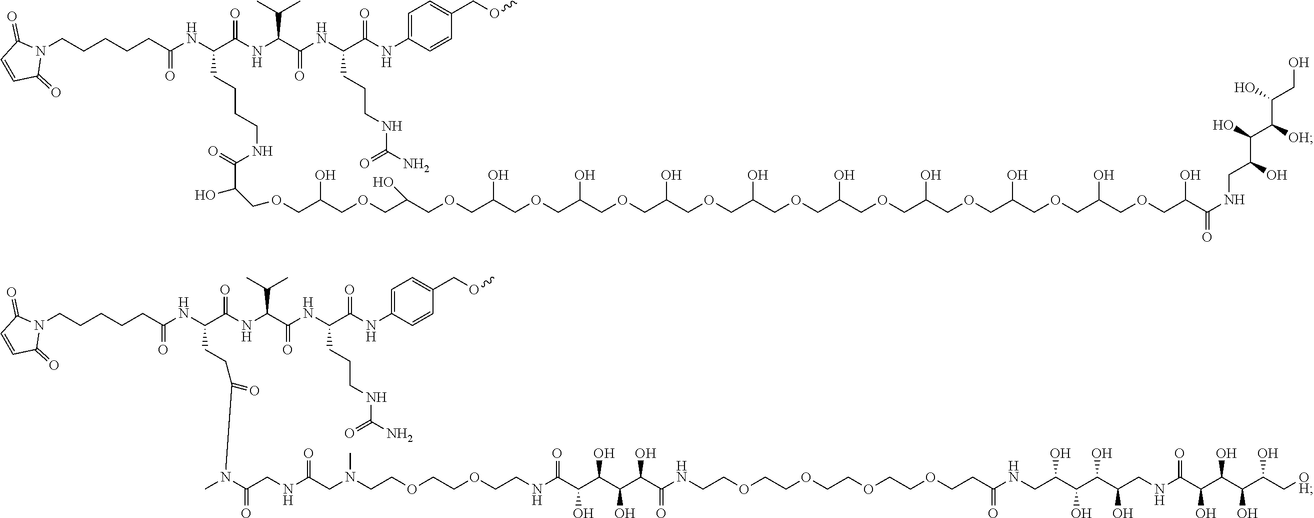 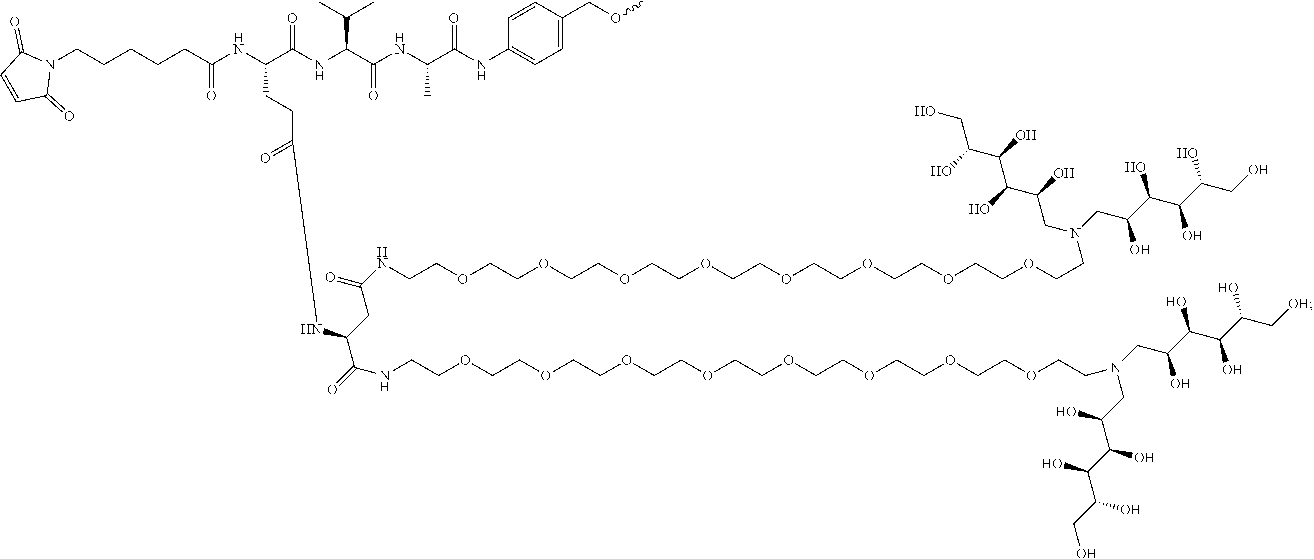 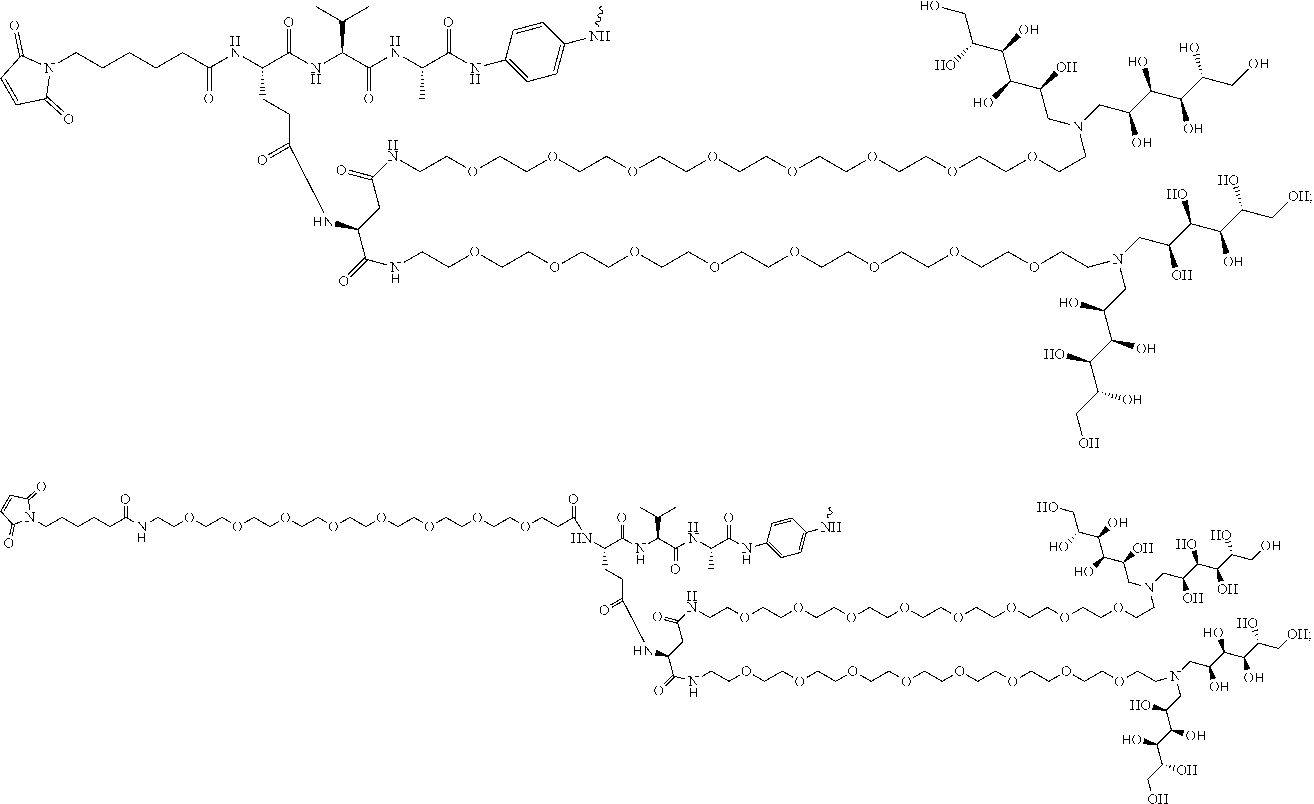 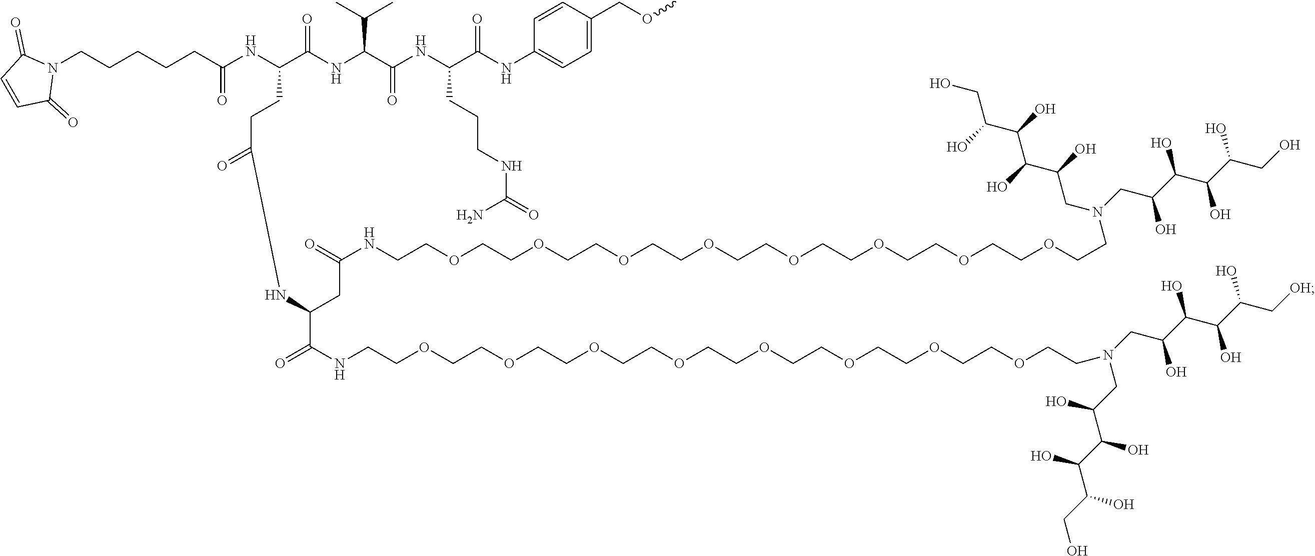 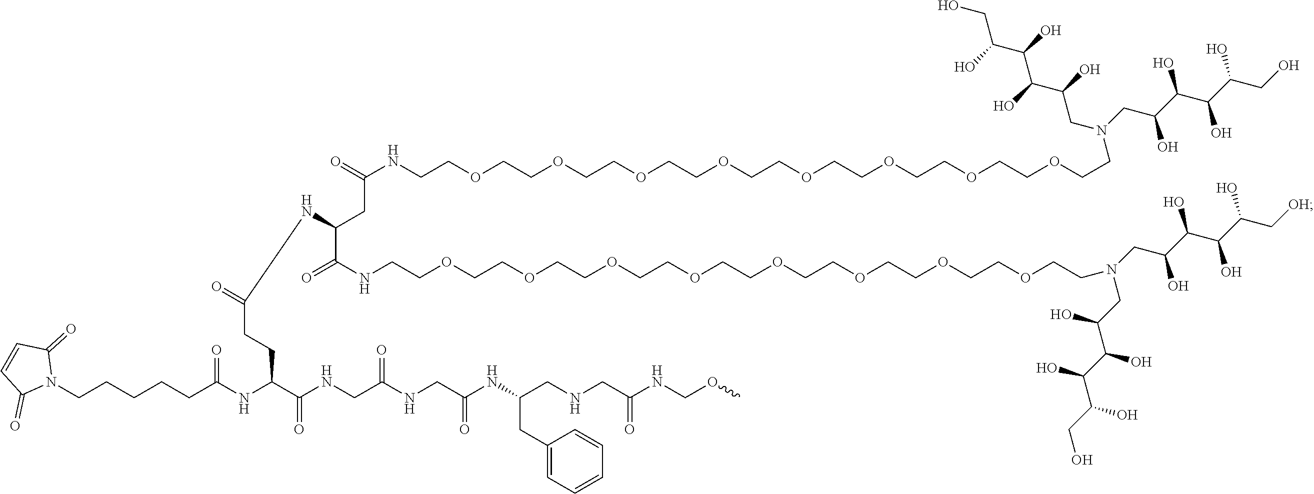 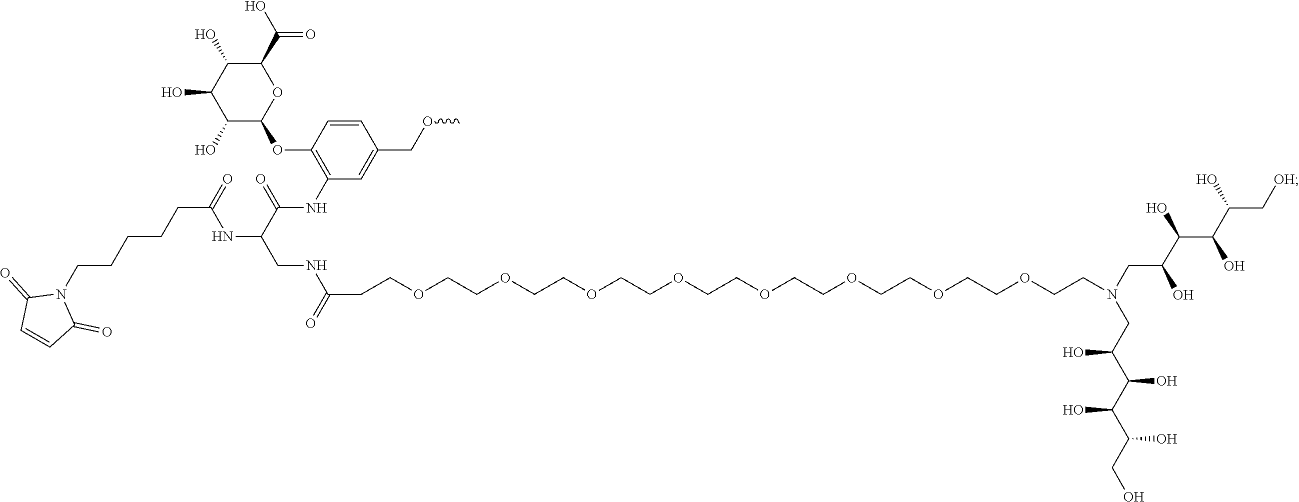 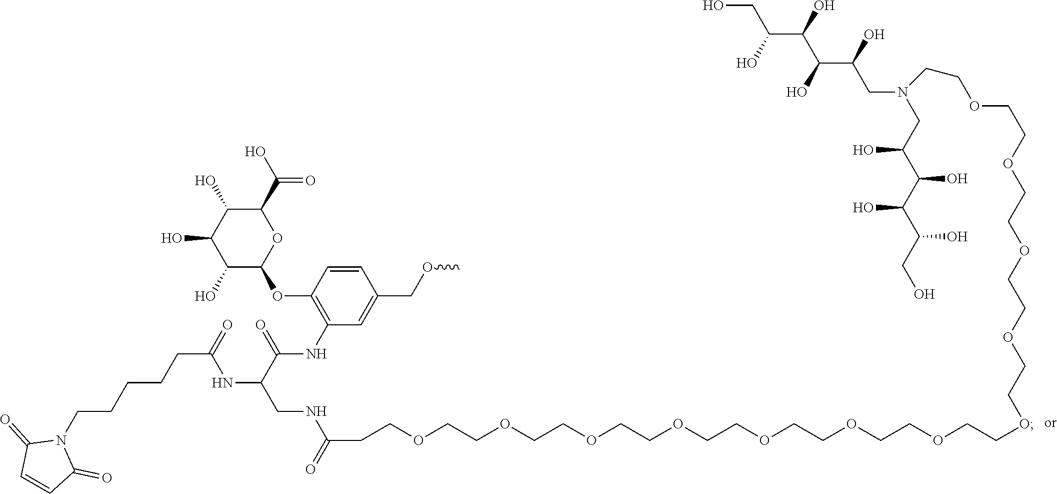 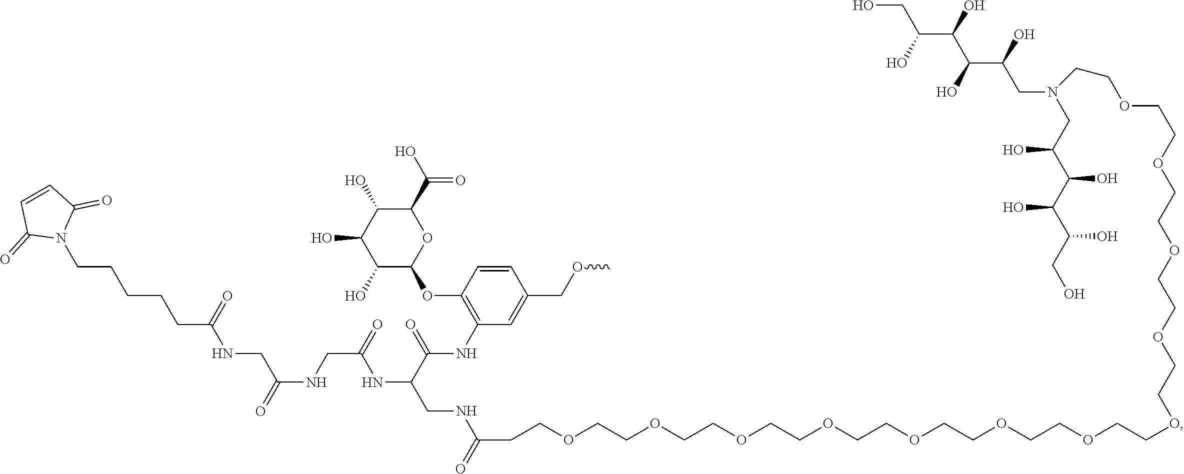
a stereoisomer thereof, wherein the wavy line [symbol] indicates an attachment site to the drug unit or for a linking group attached to the drug unit.

52. The conjugate of claim 24, wherein the drug unit is attached to the linker compound, to form a drug-linker compound, which can be attached to the bispecific binding agent to form the conjugate.

53. The conjugate of claim 52, wherein the drug unit is selected from a cytotoxic agent, an immune modulatory agent, a nucleic acid, a growth inhibitory agent, a PROTAC, a toxin, a radioactive isotope, and a chelating ligand.

54. The conjugate of claim 23, wherein an average drug loading (p^{load}) of the conjugate is from about 1 to about 8, about 2, about 3, about 4, about 5, about 6, about 7, about 8, about 10, about 12, about 14, about 16, about 3 to about 5, about 6 to about 8, or about 8 to about 16.

55. The conjugate of claim 23, selected from the following: 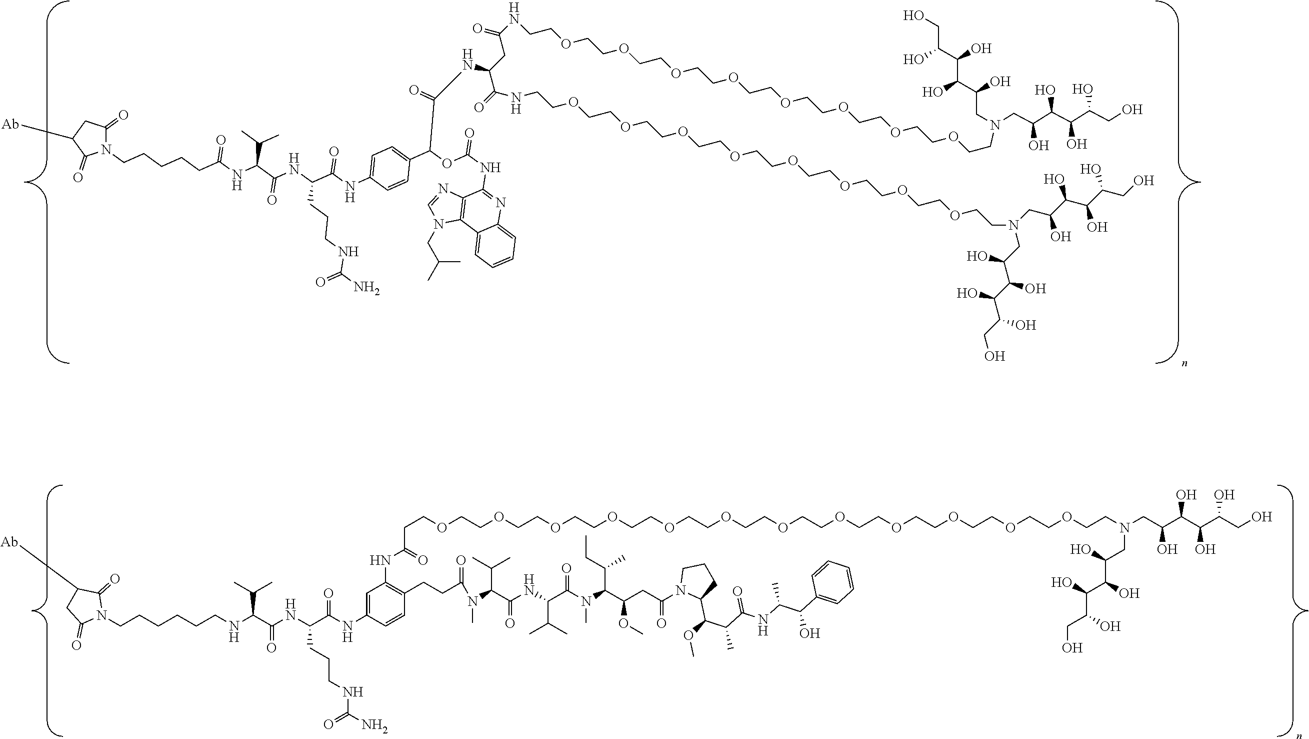 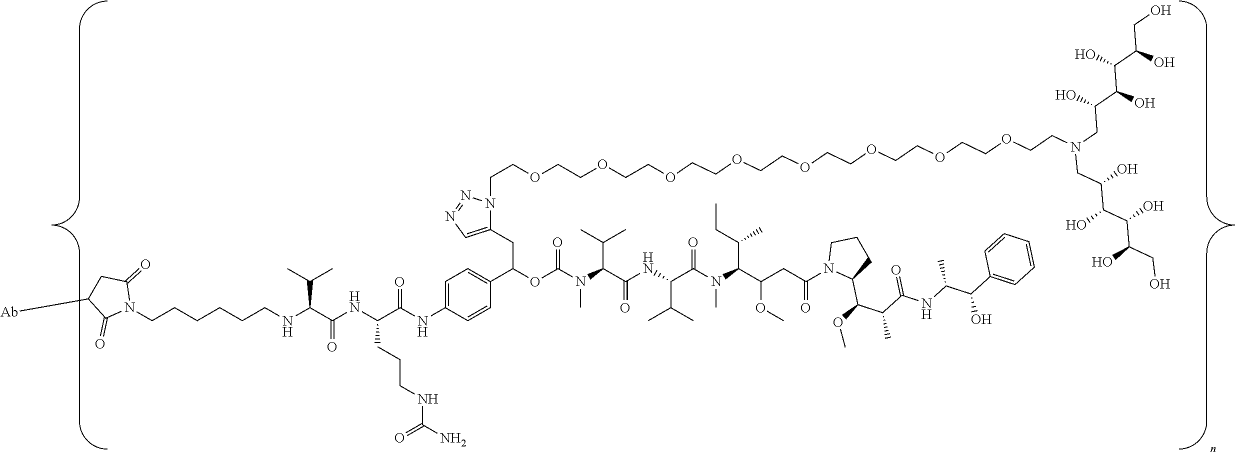 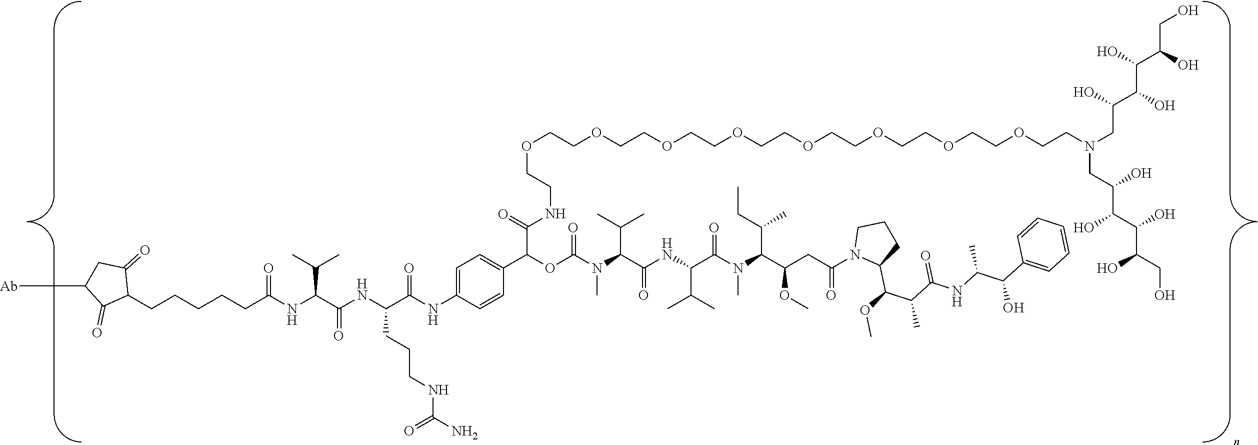 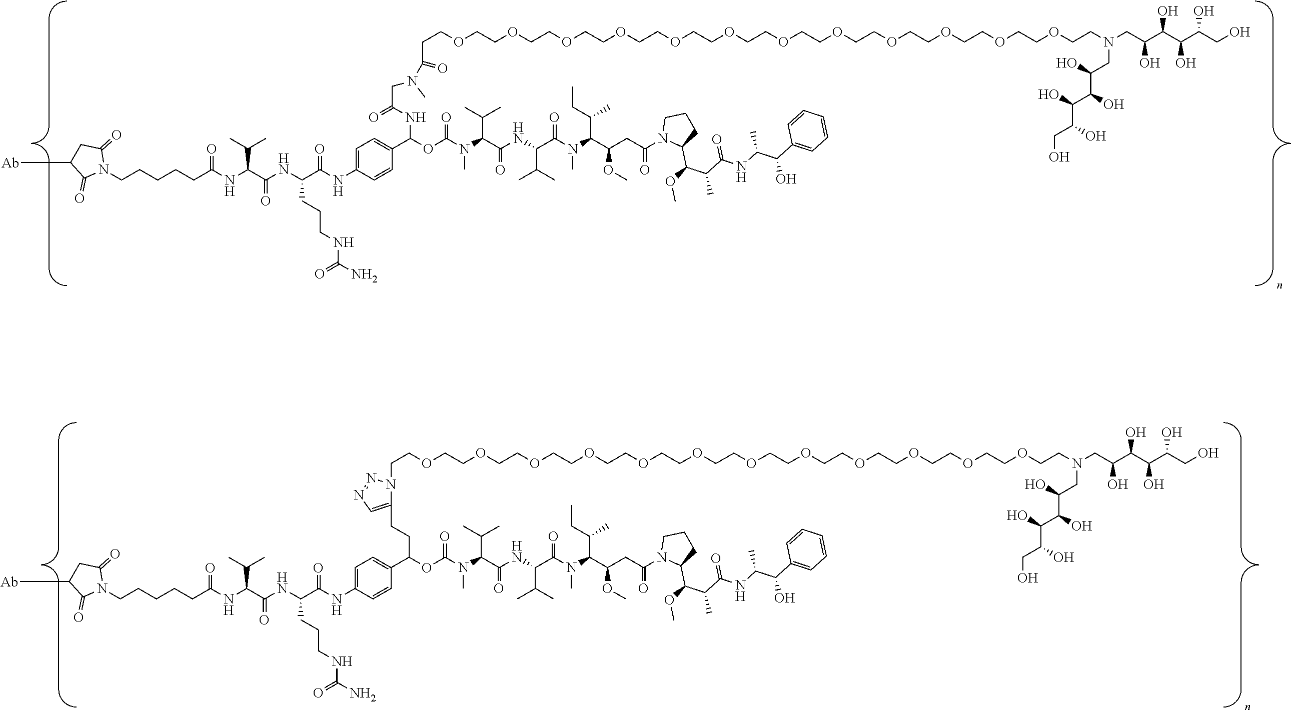 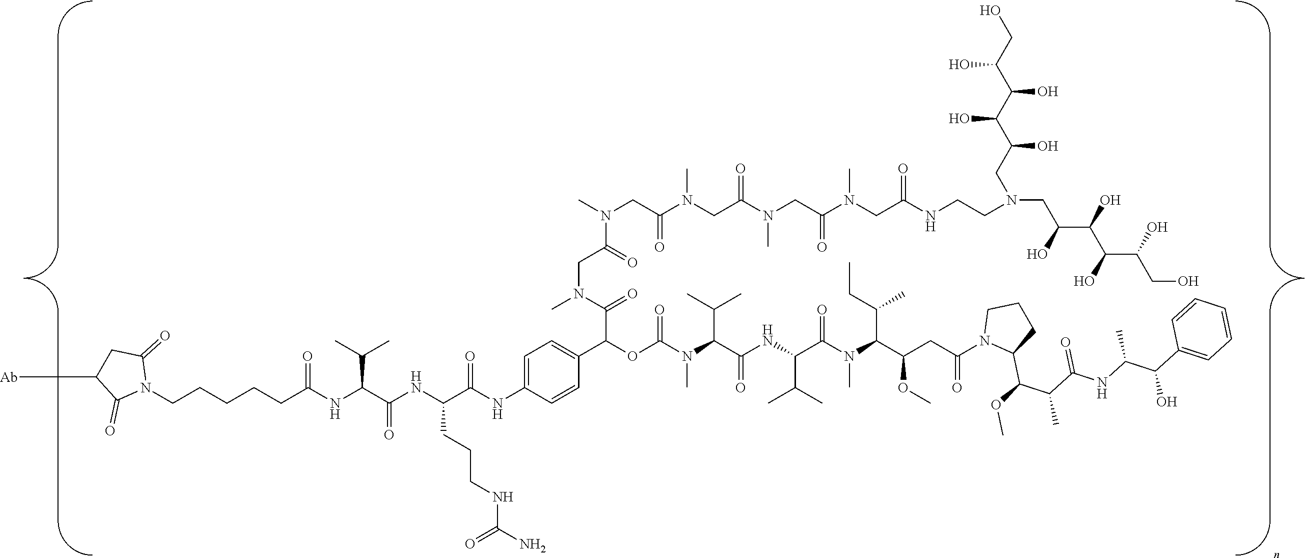 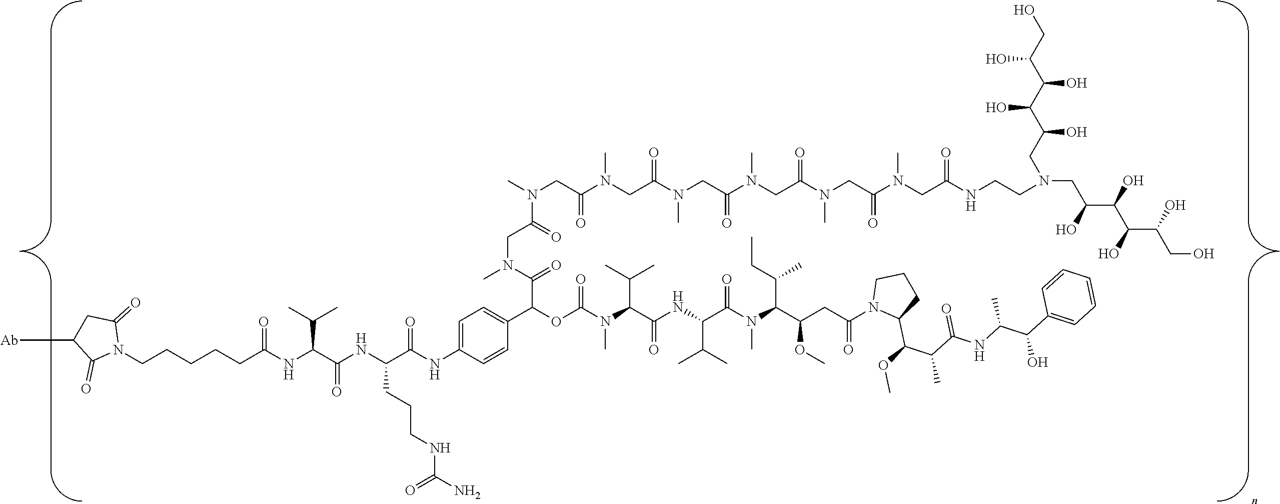 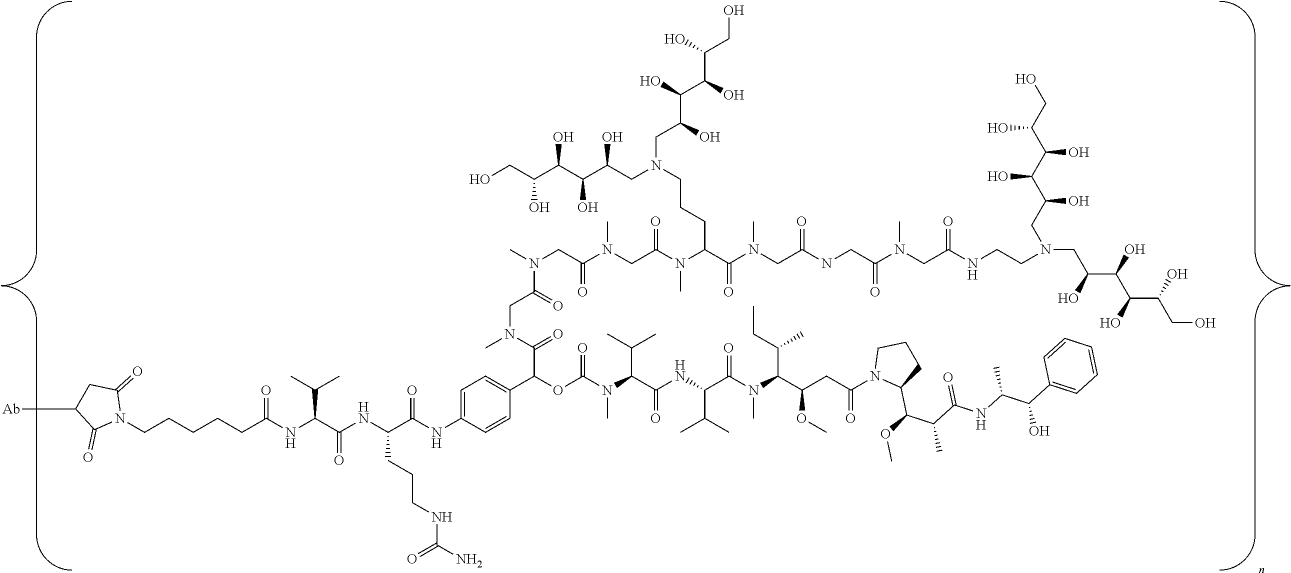 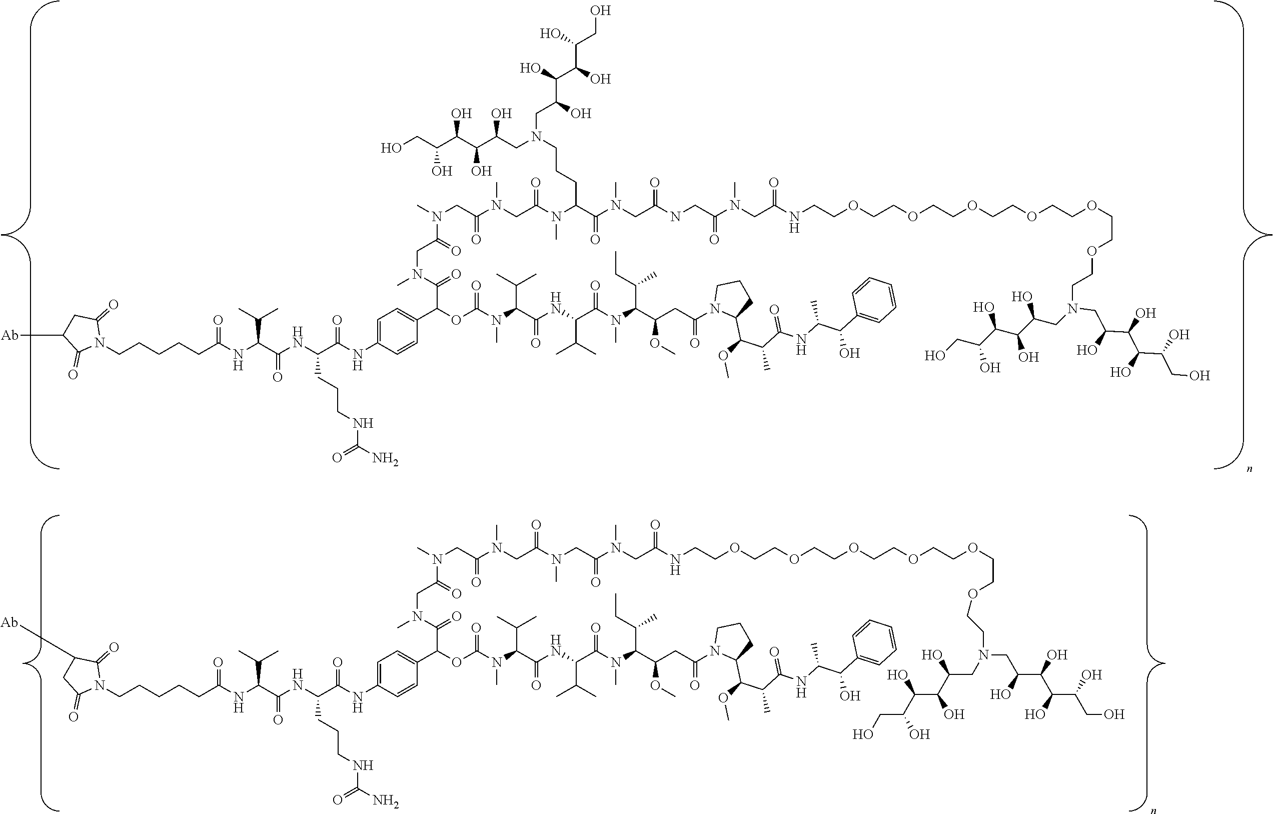 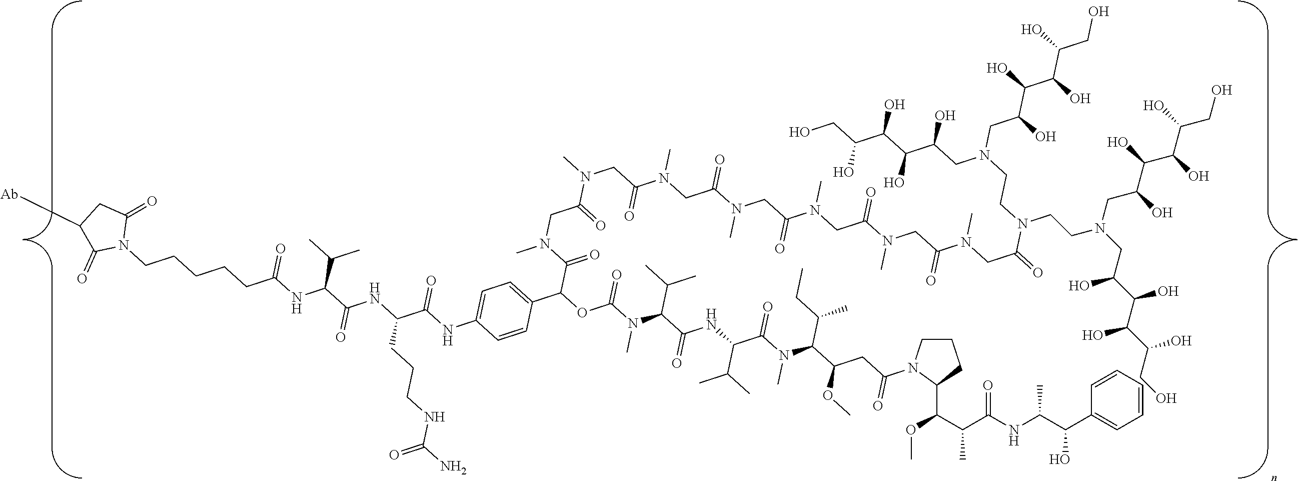 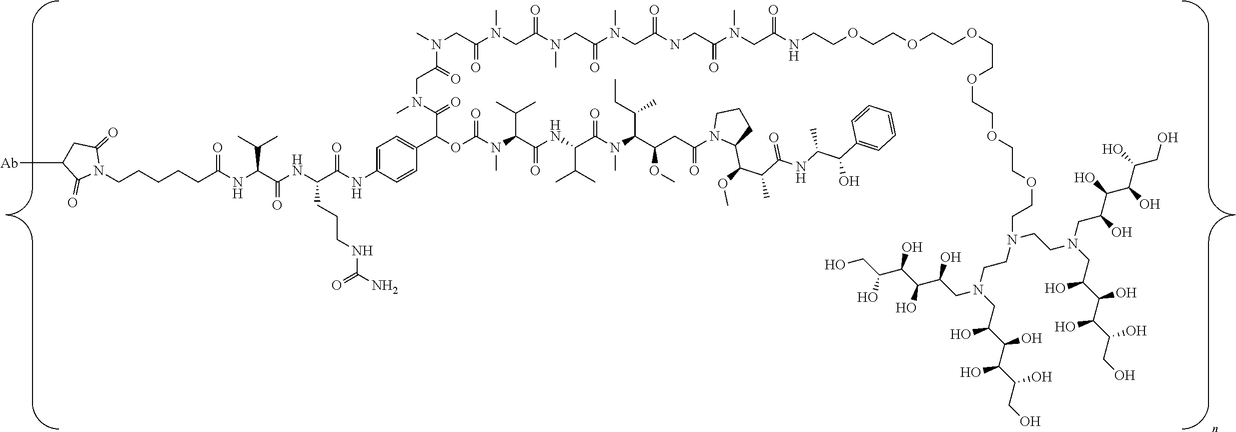 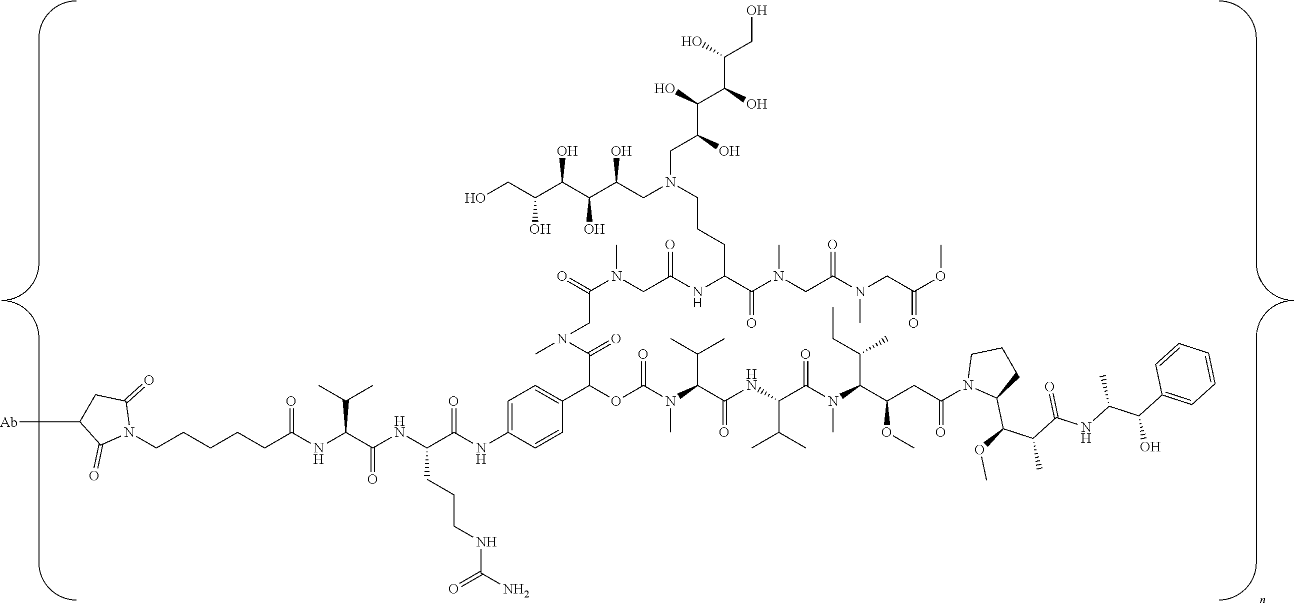 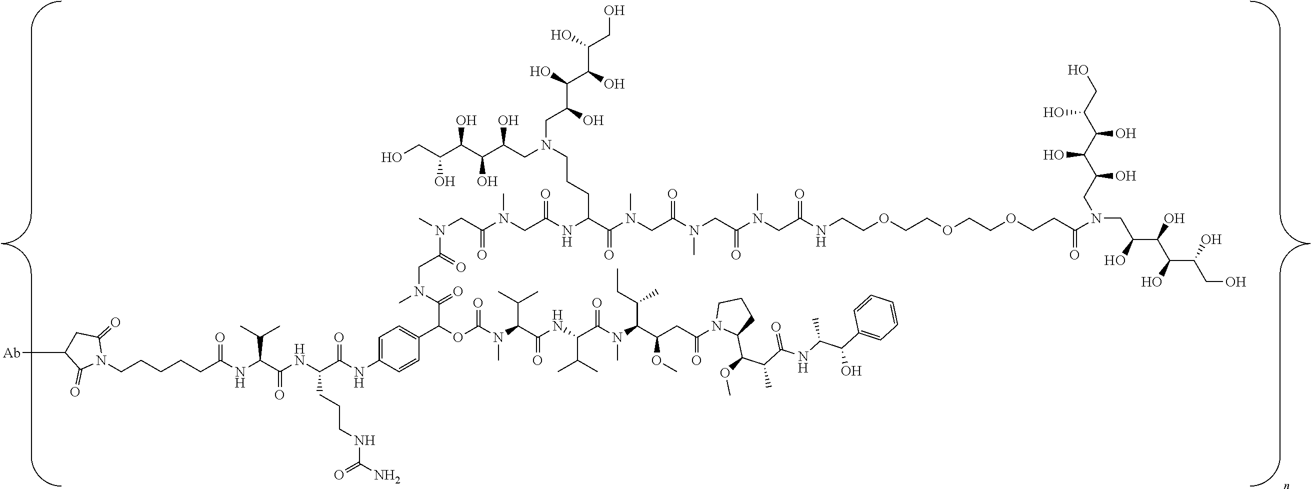 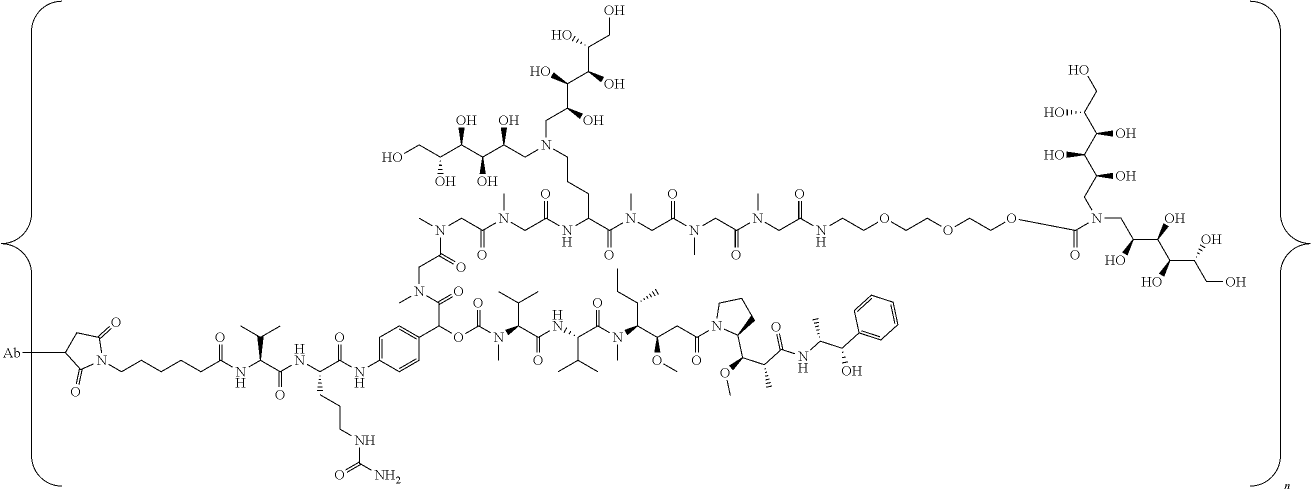 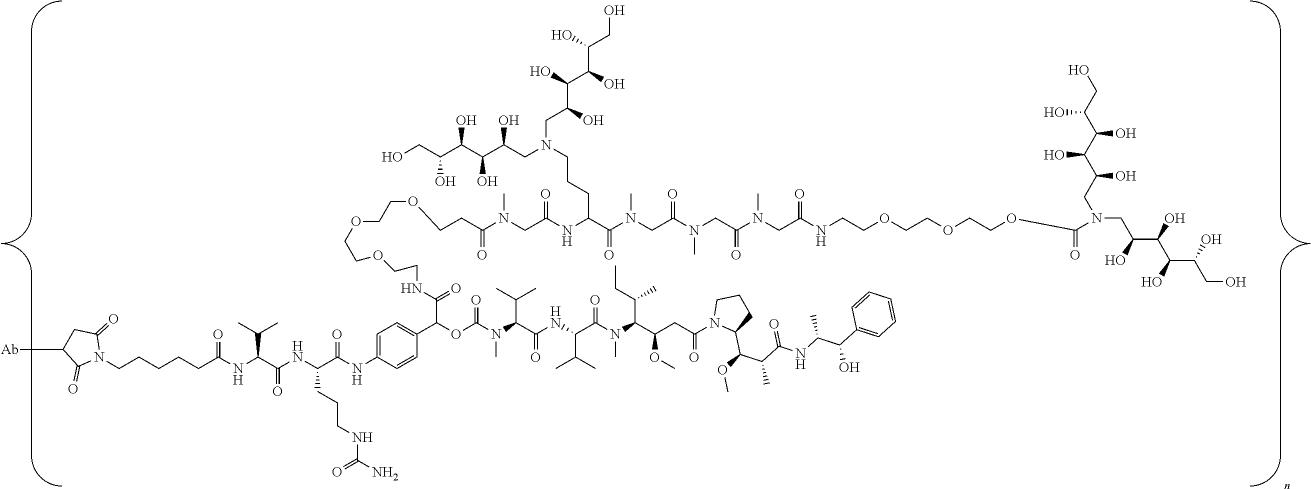 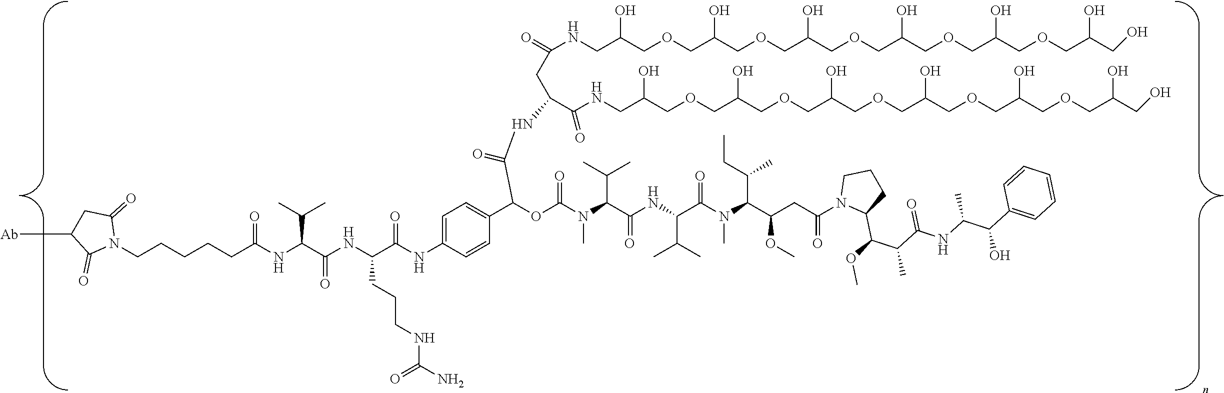 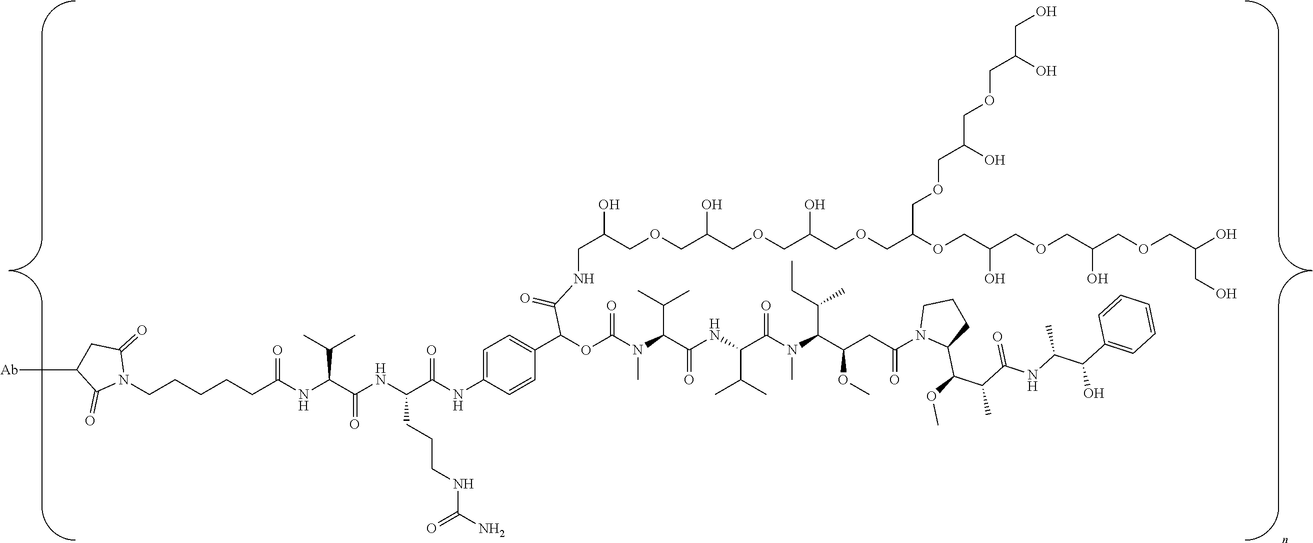 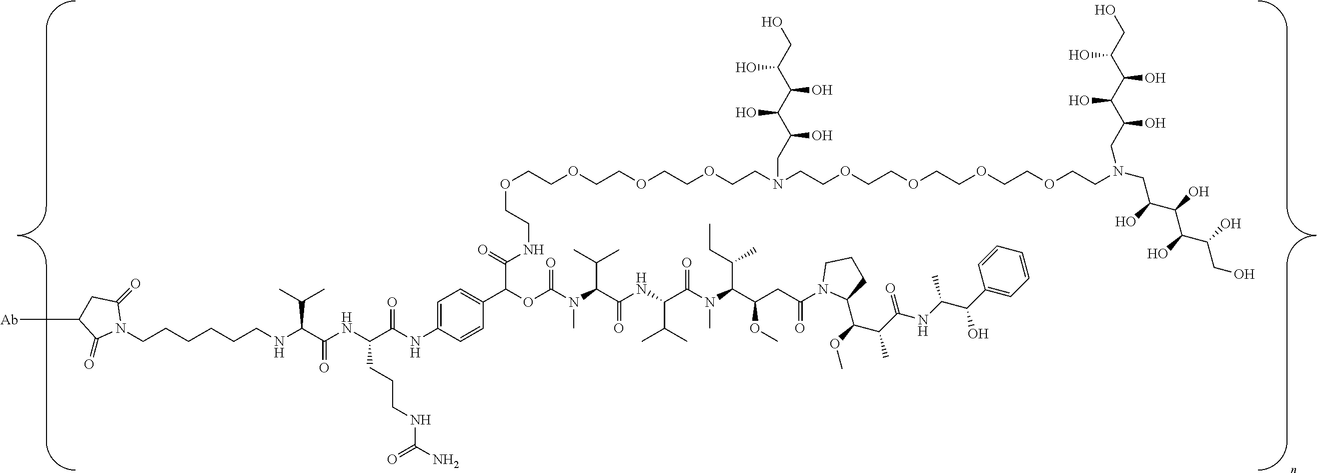 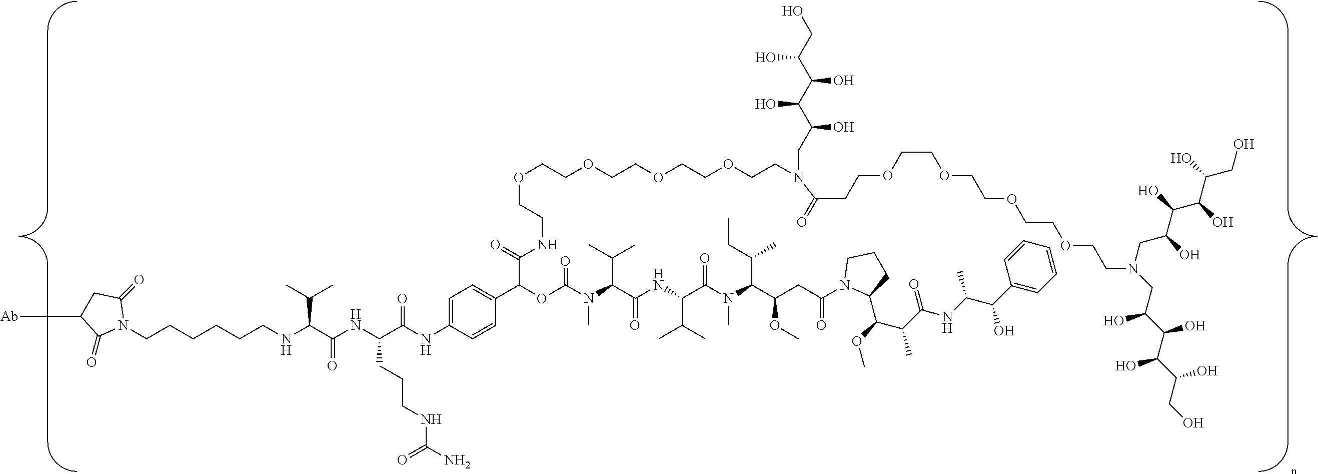 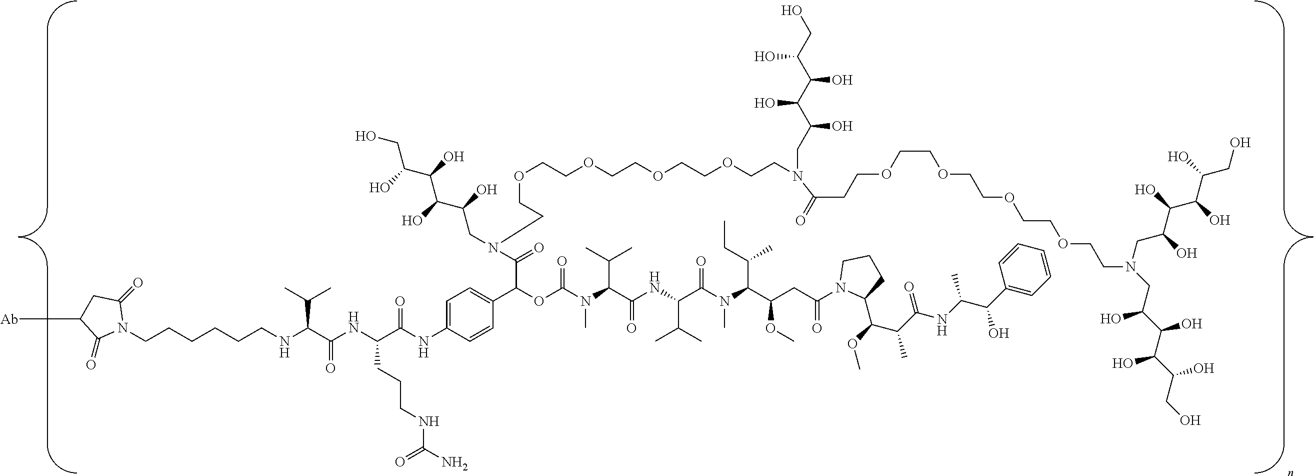 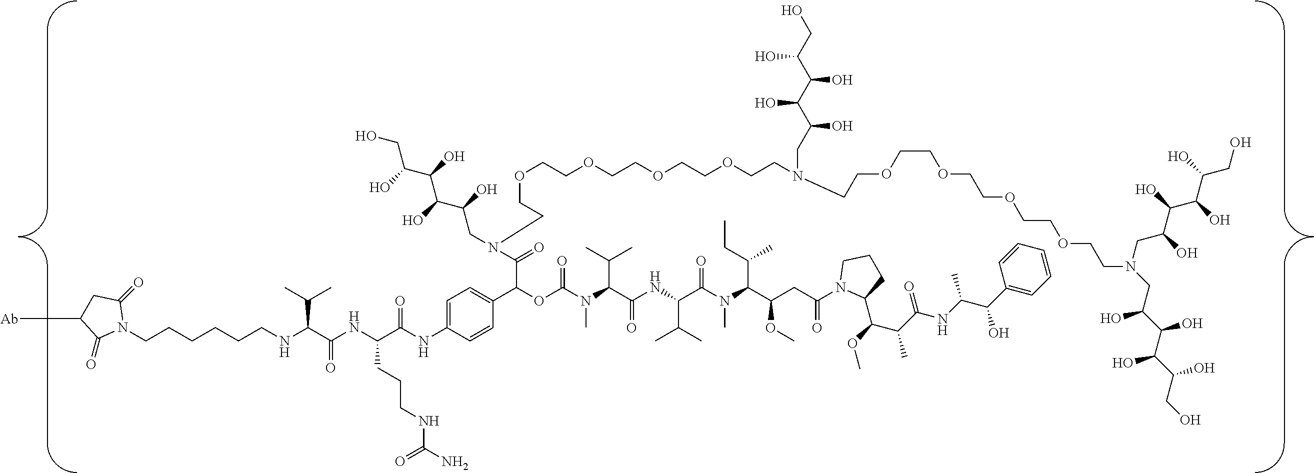 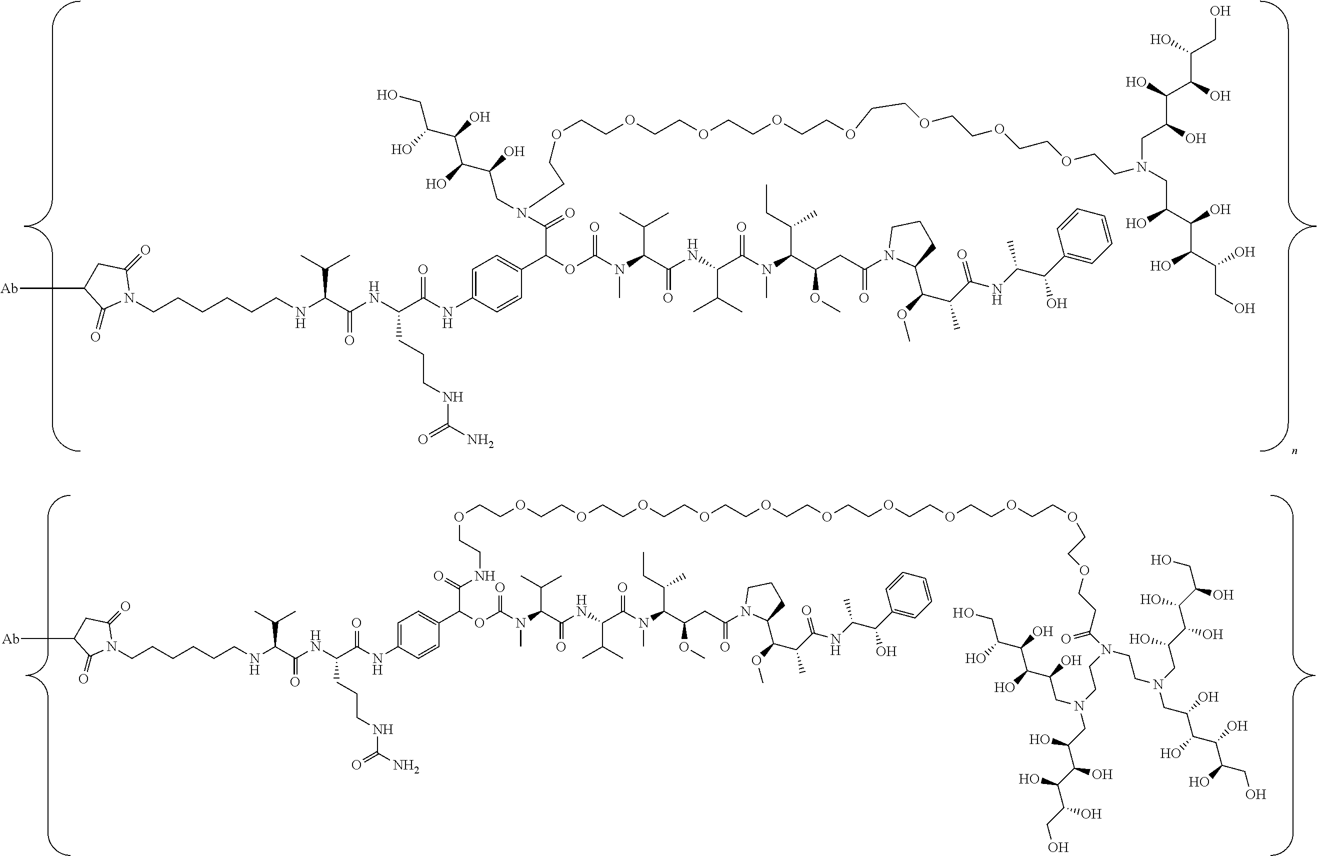 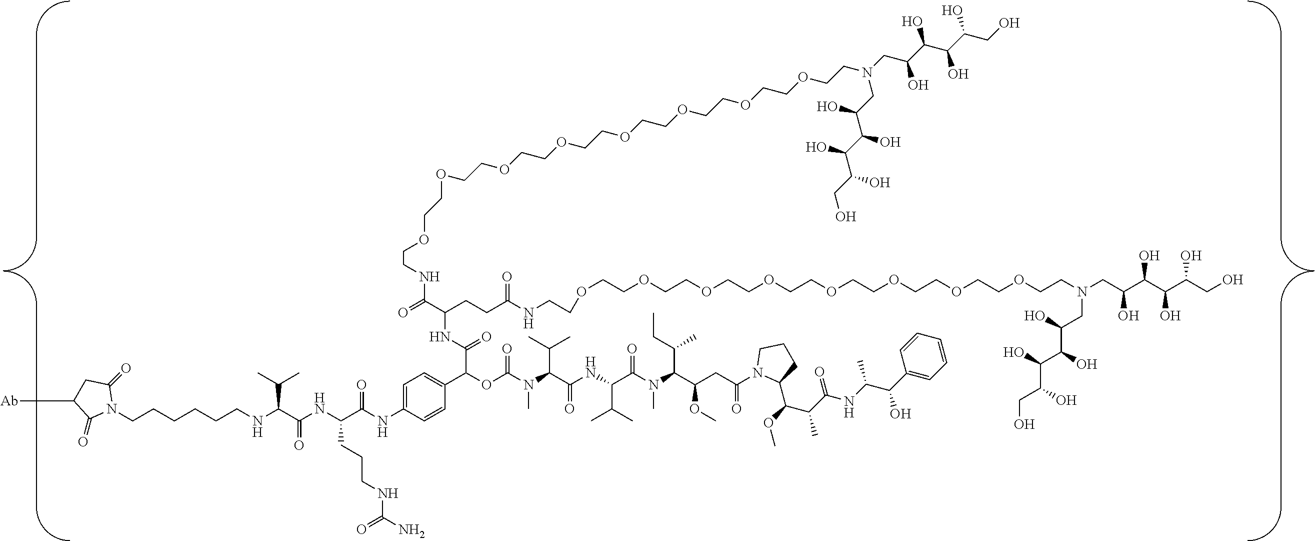 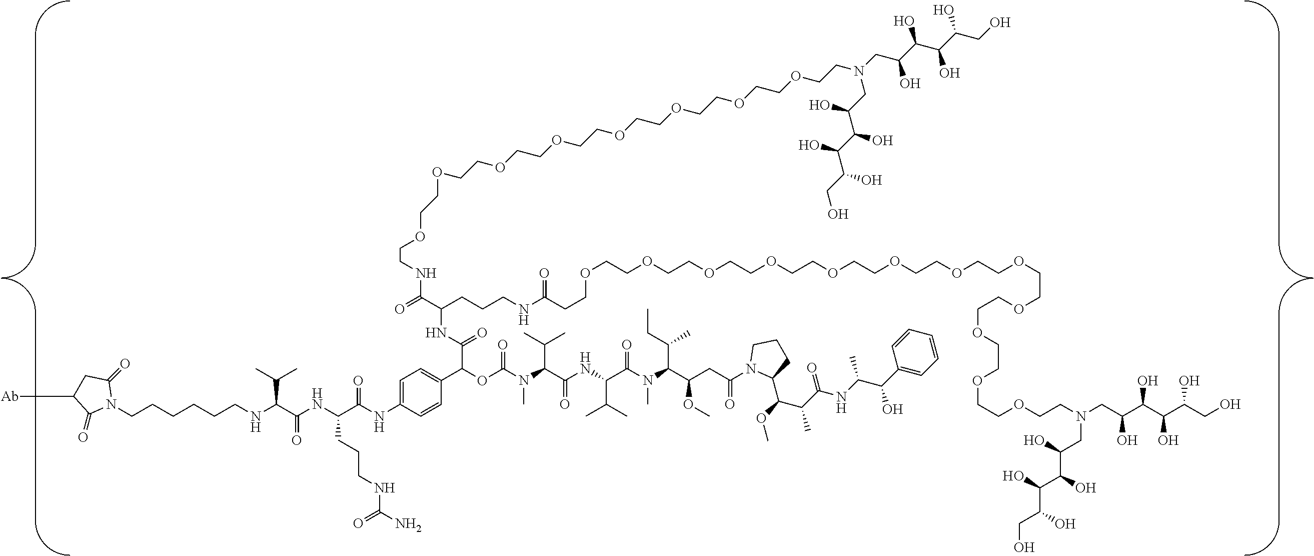 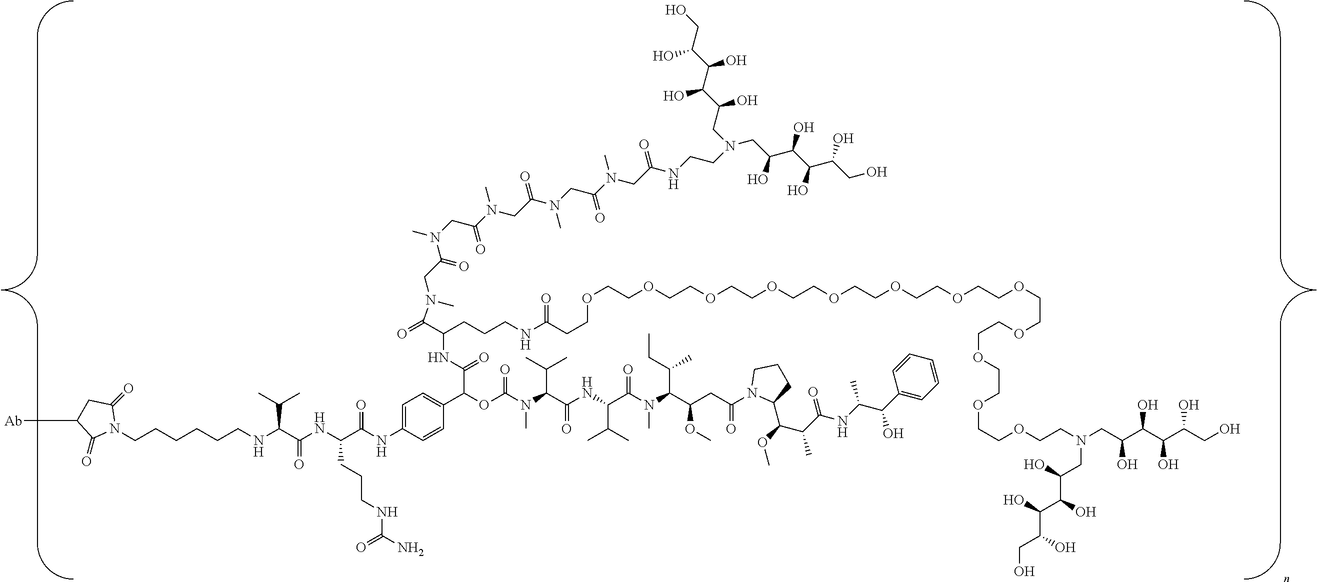 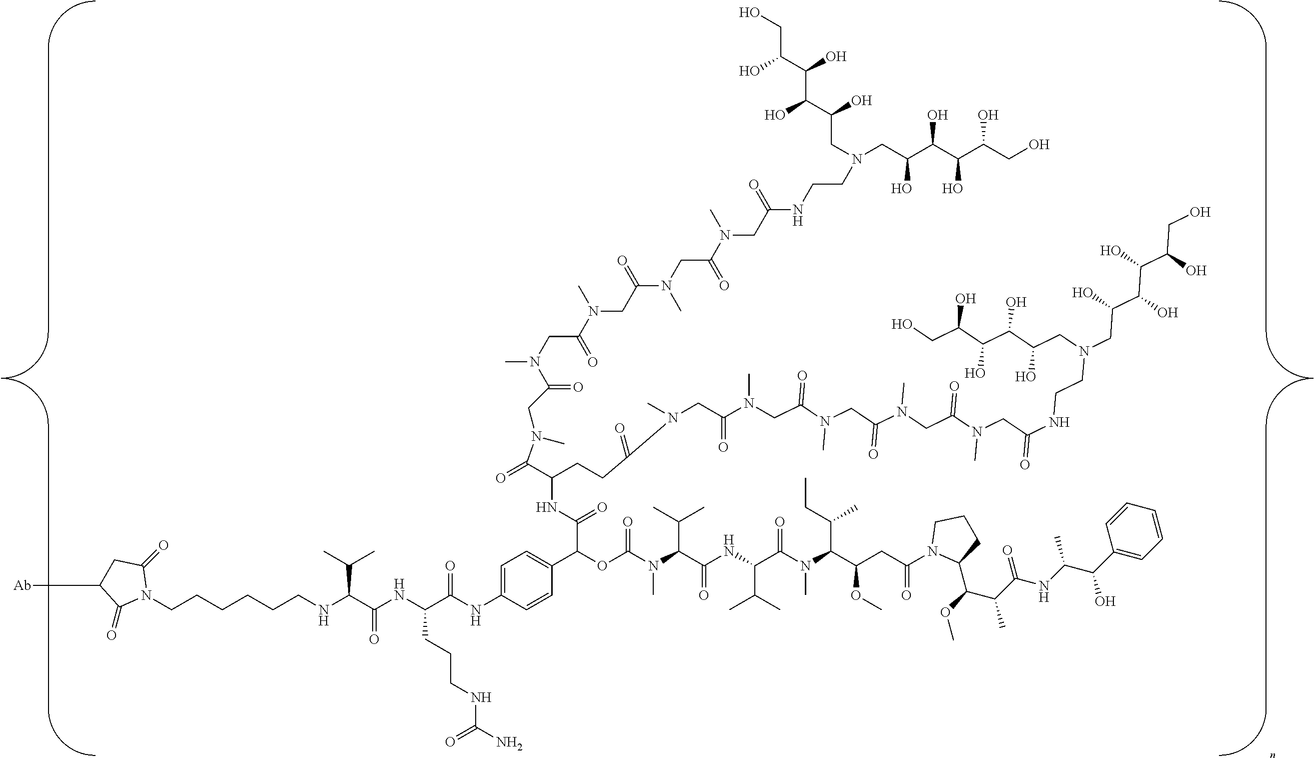 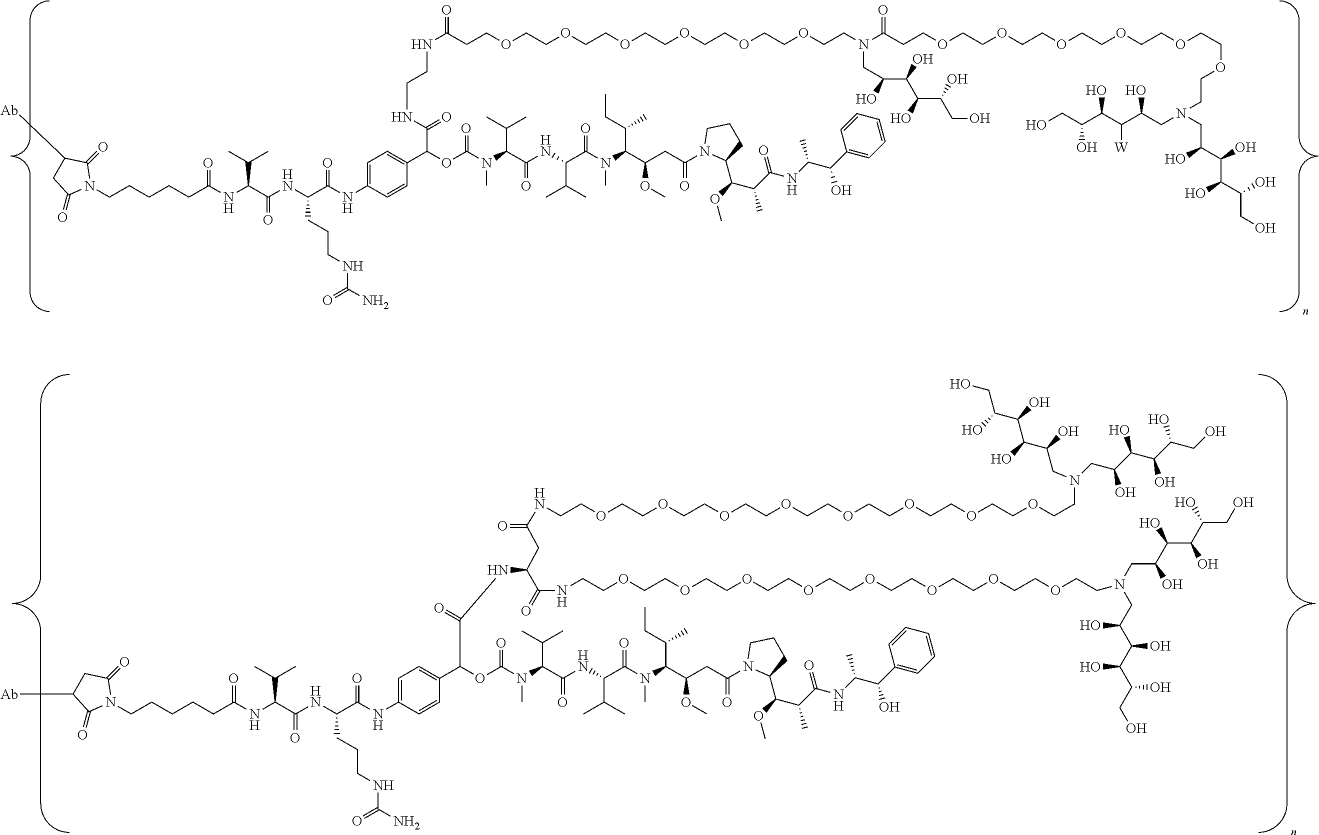 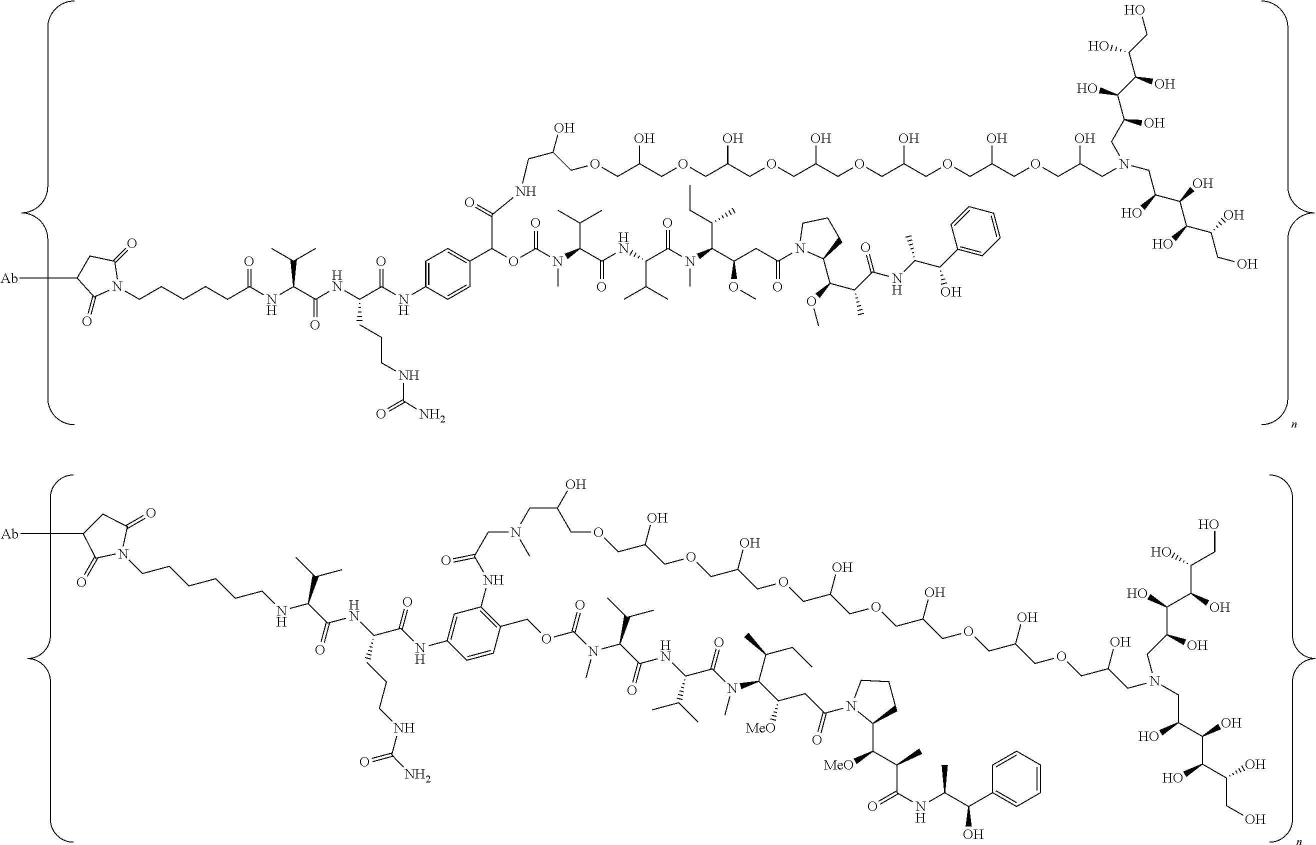 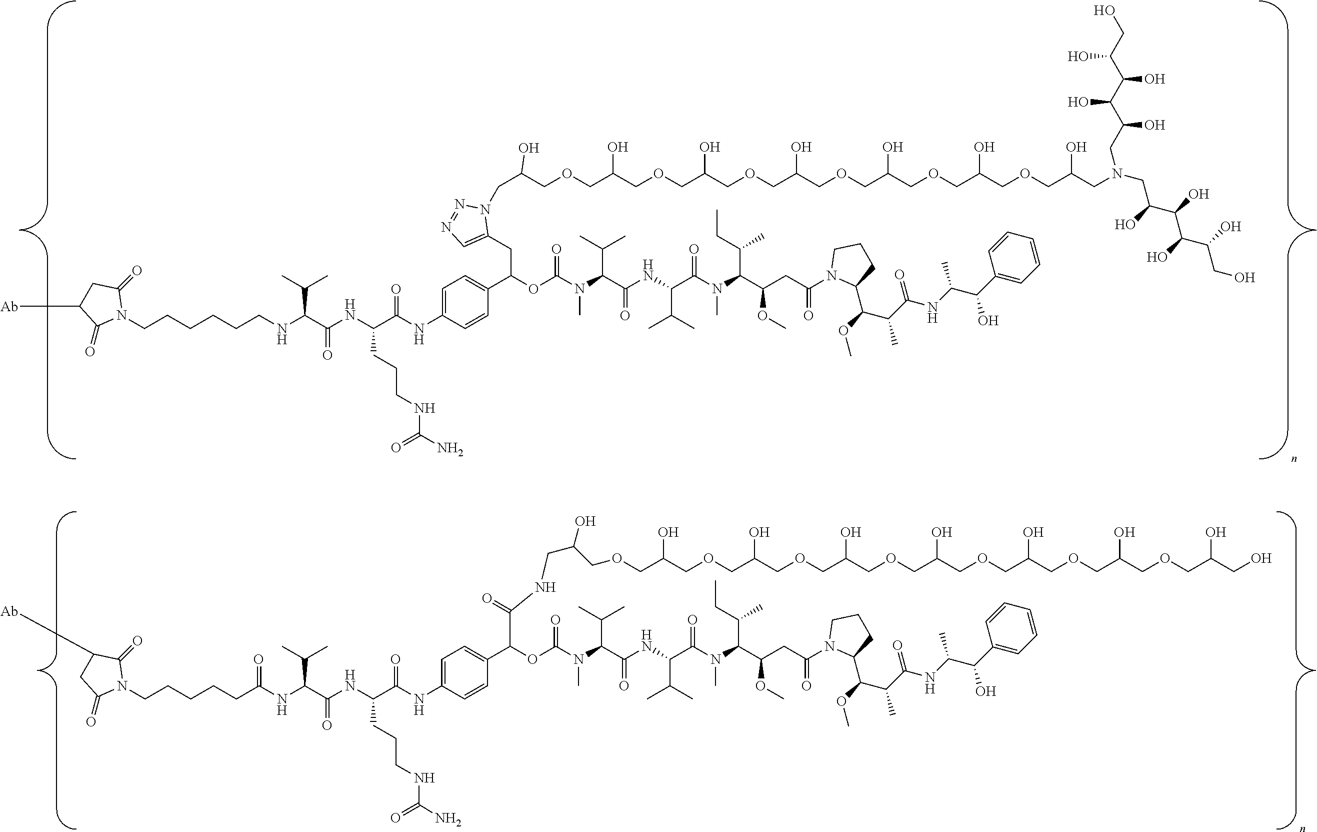 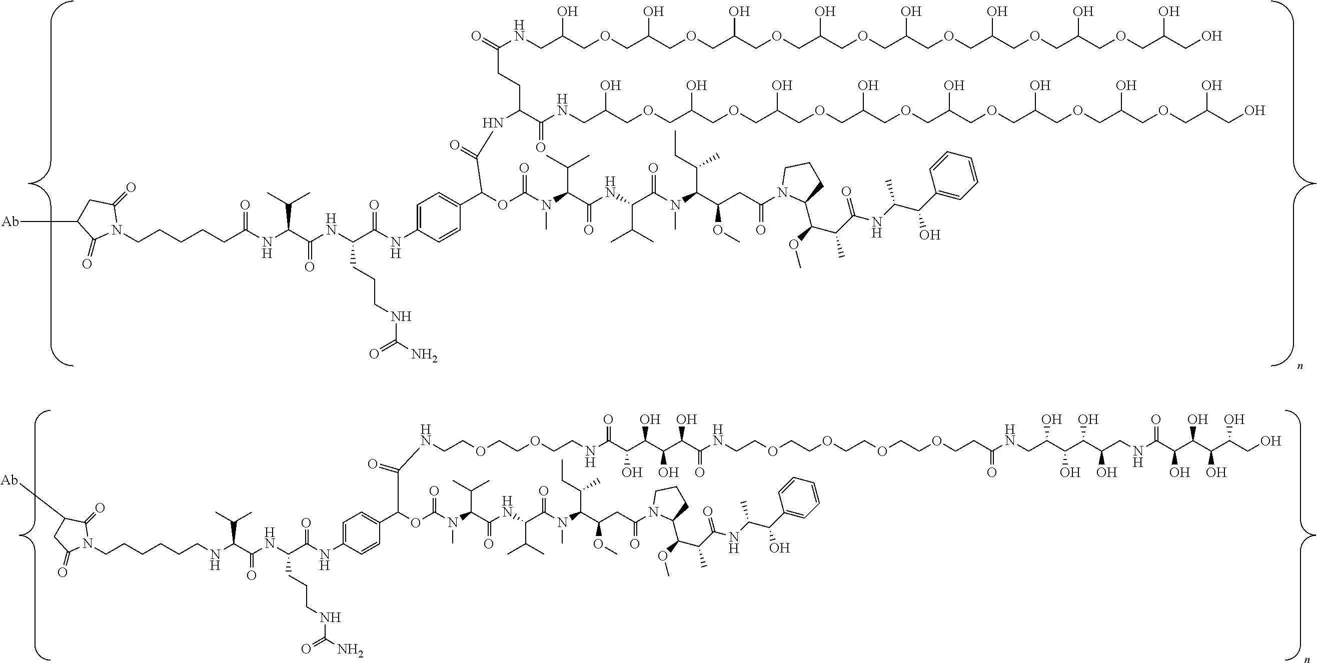 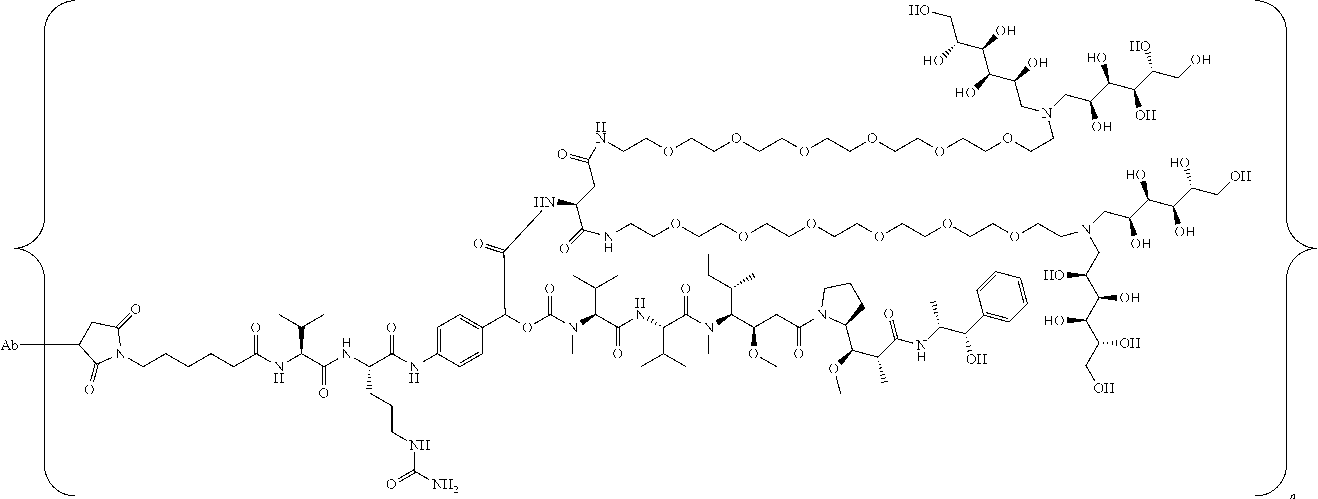 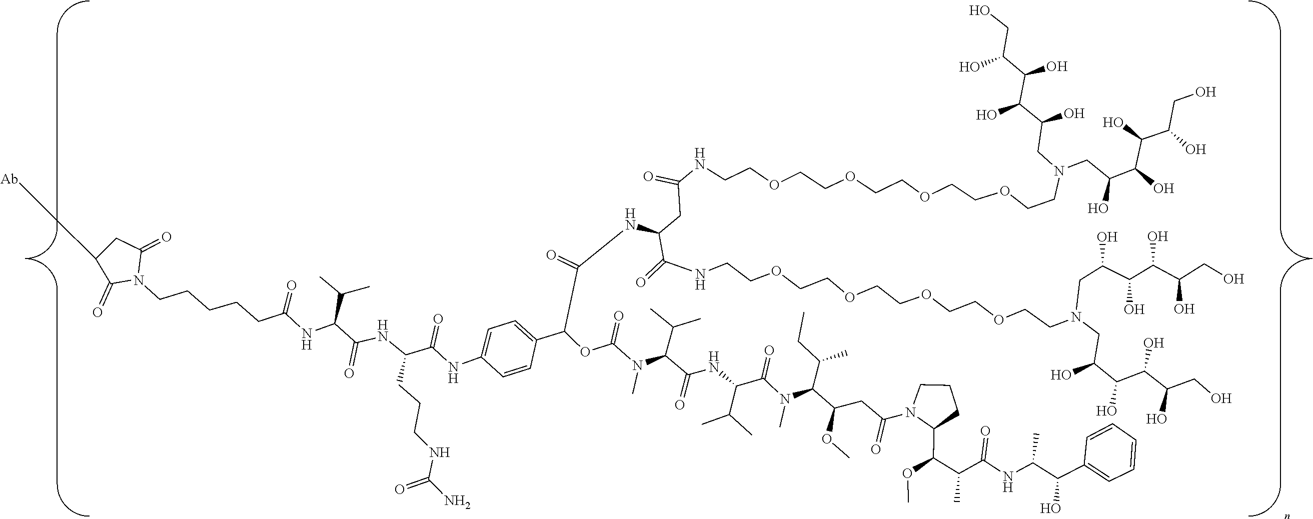 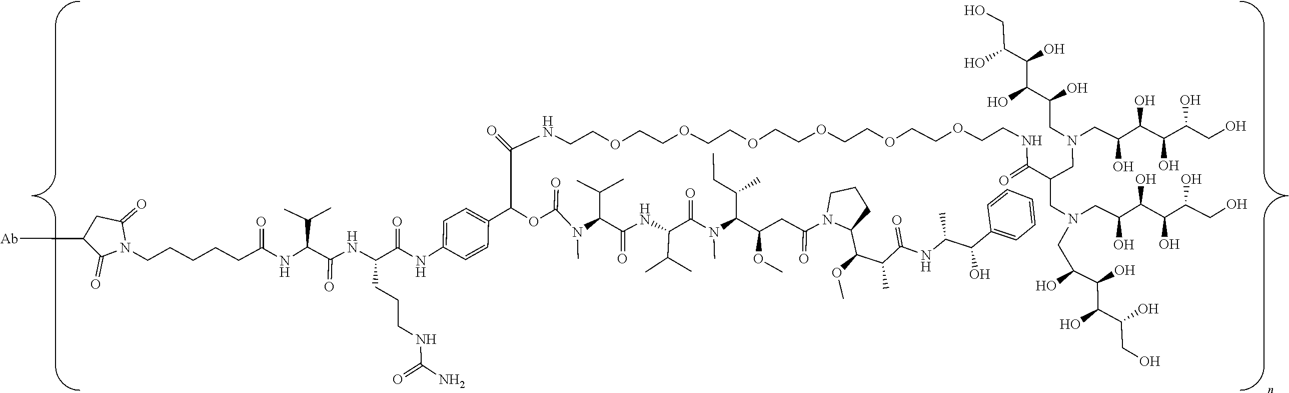 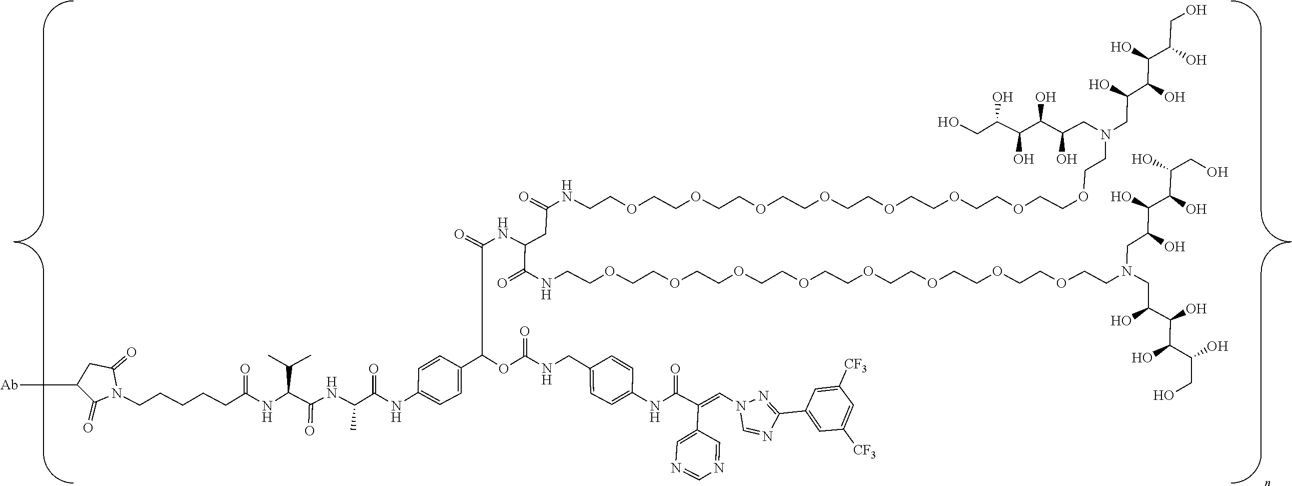 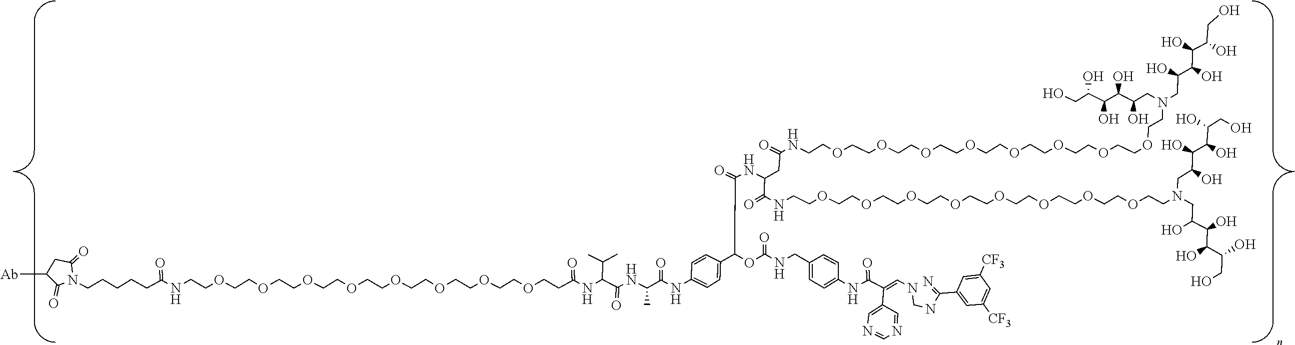 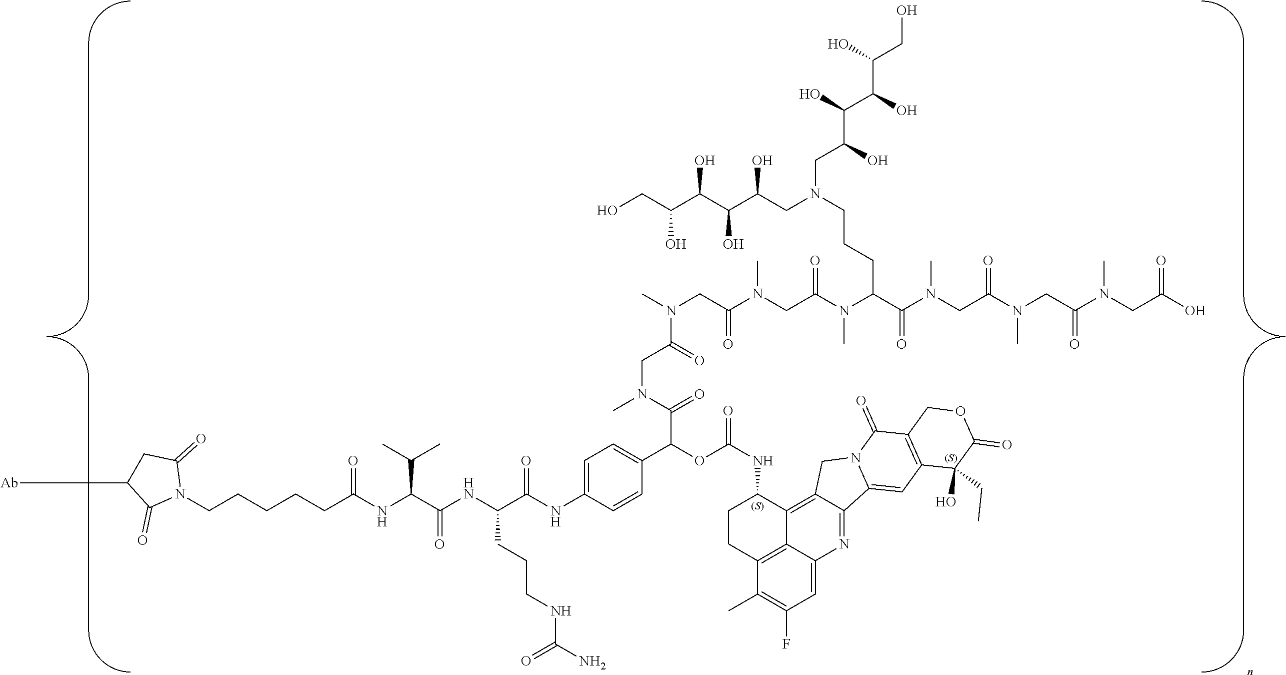 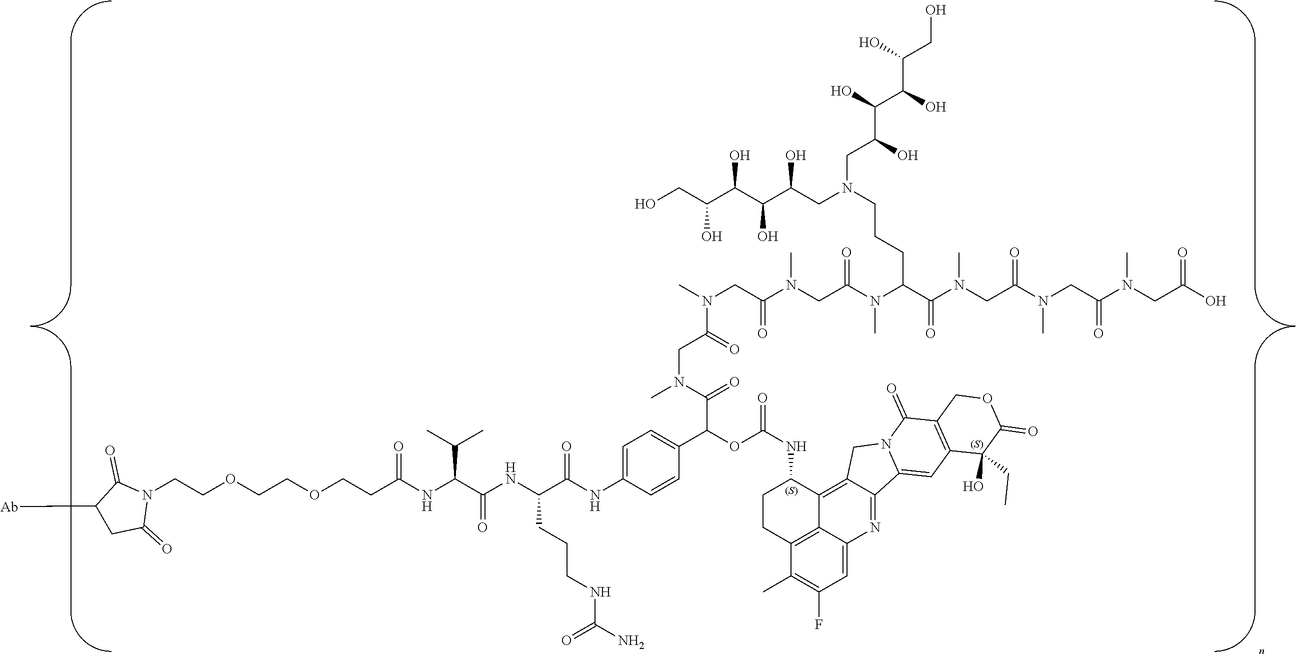 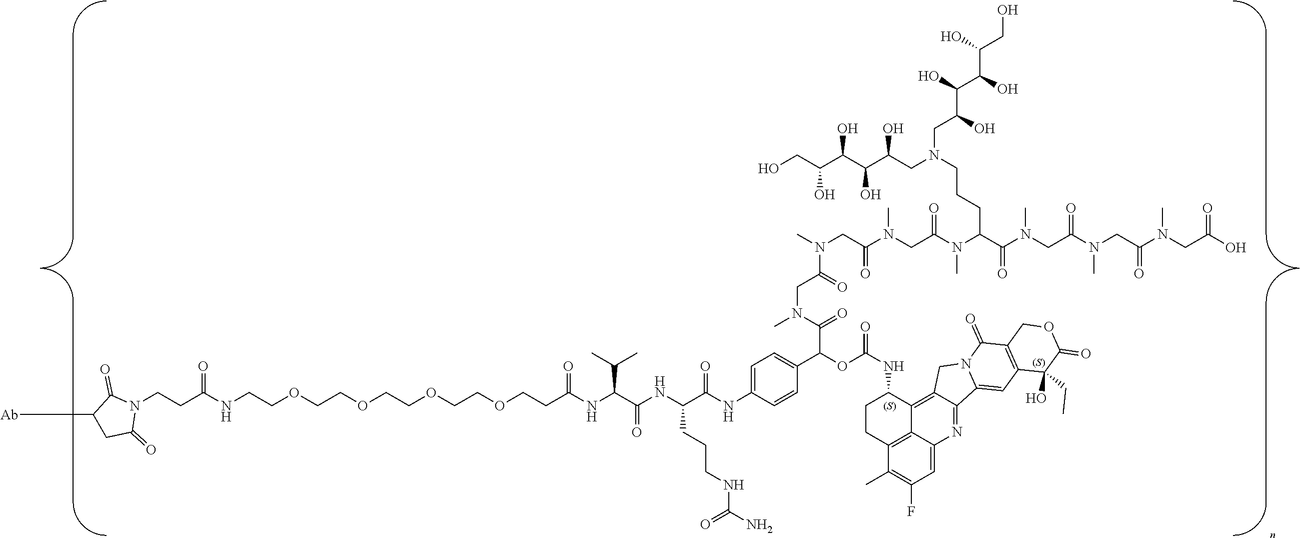 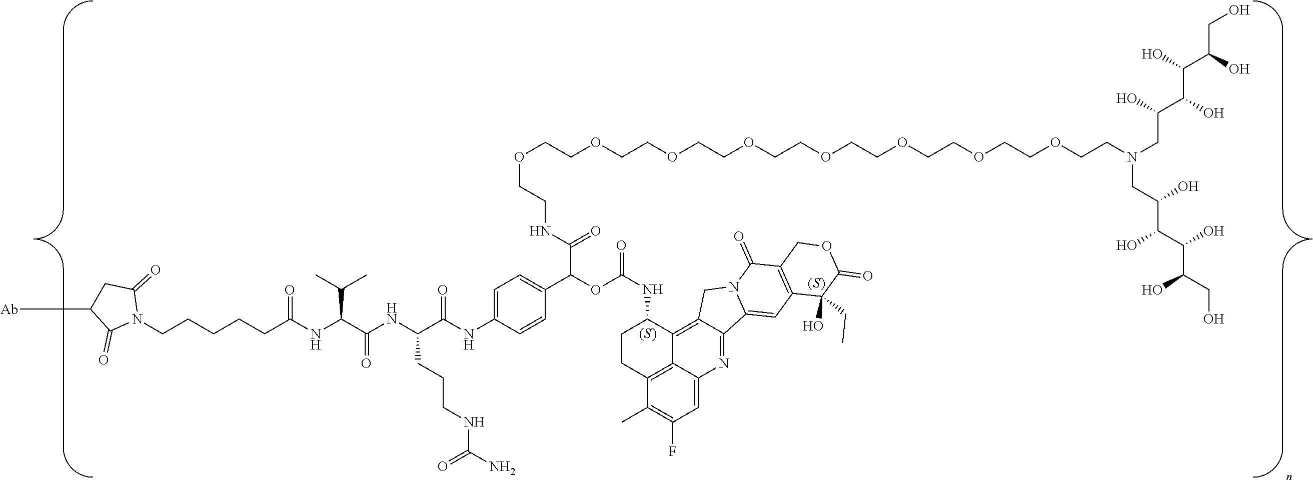 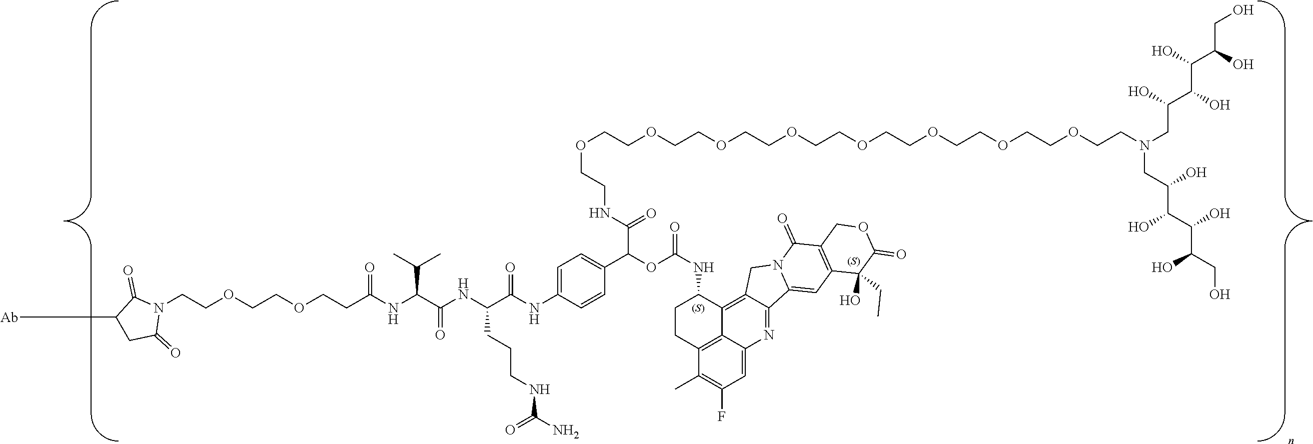 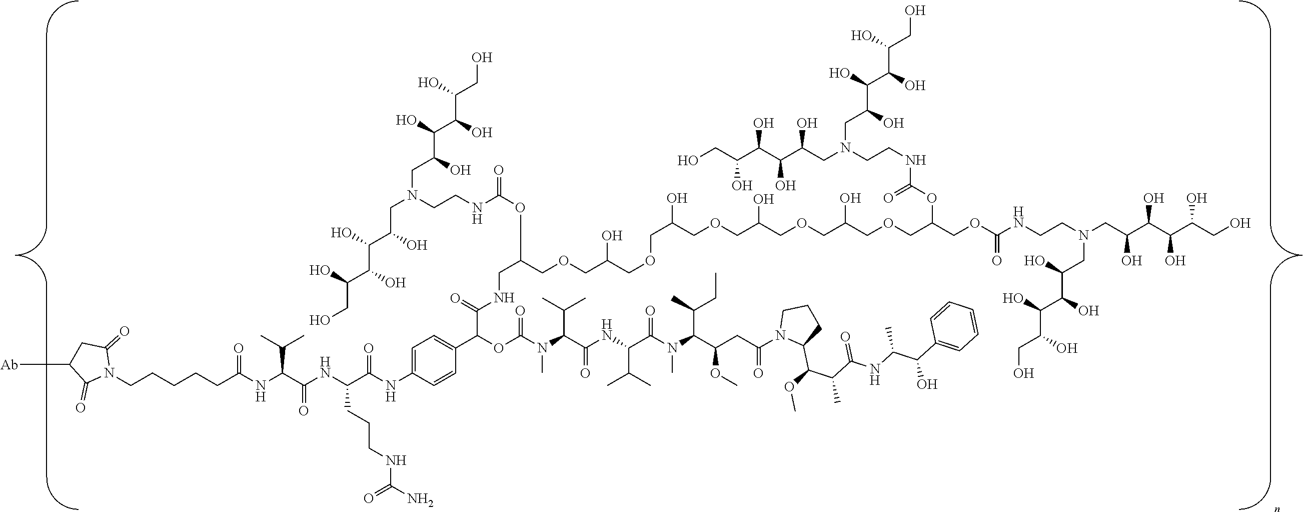 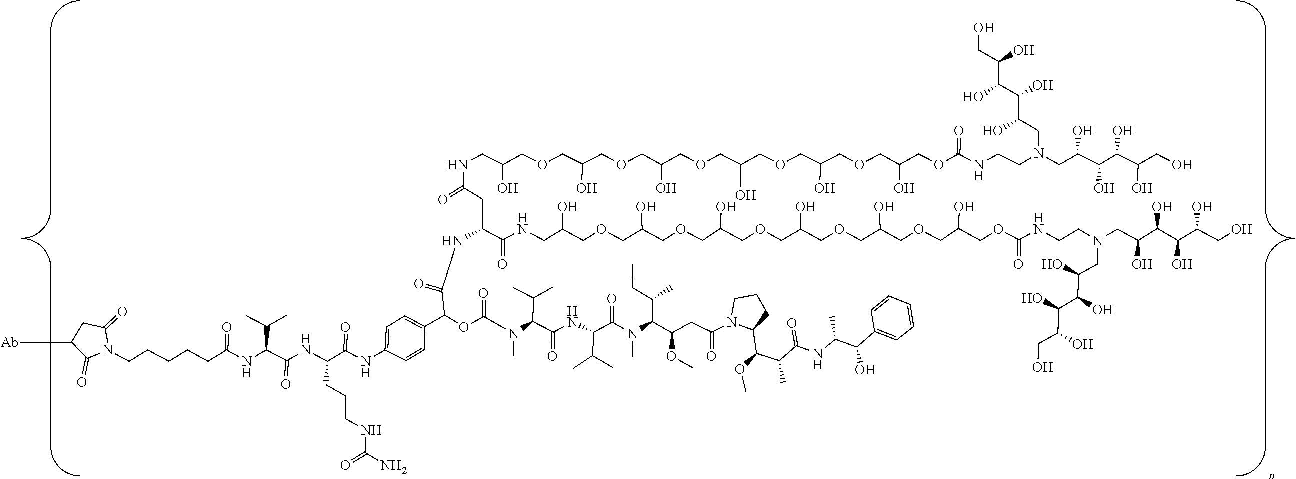 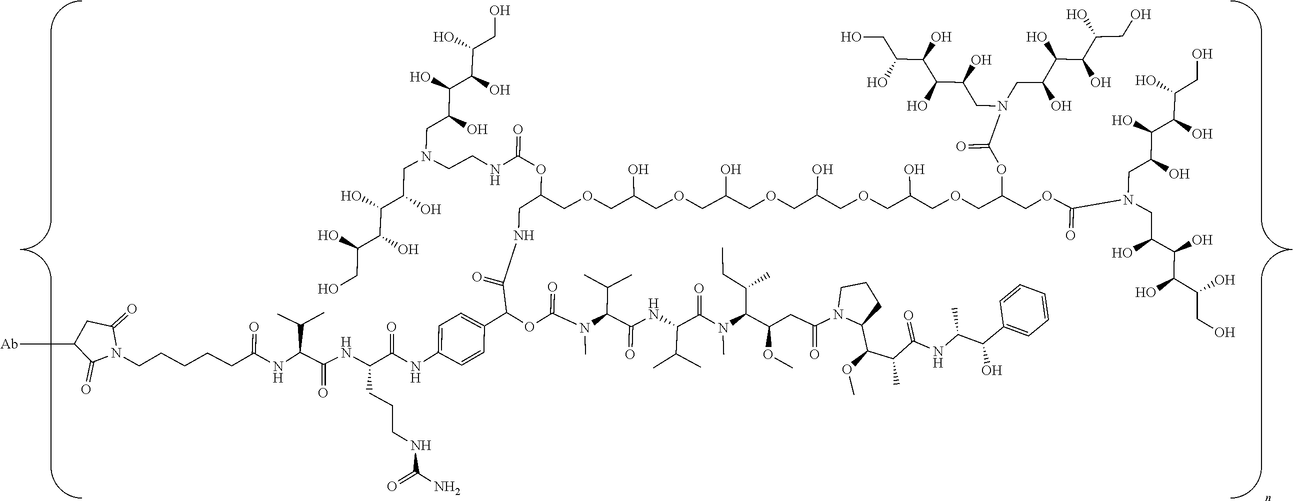 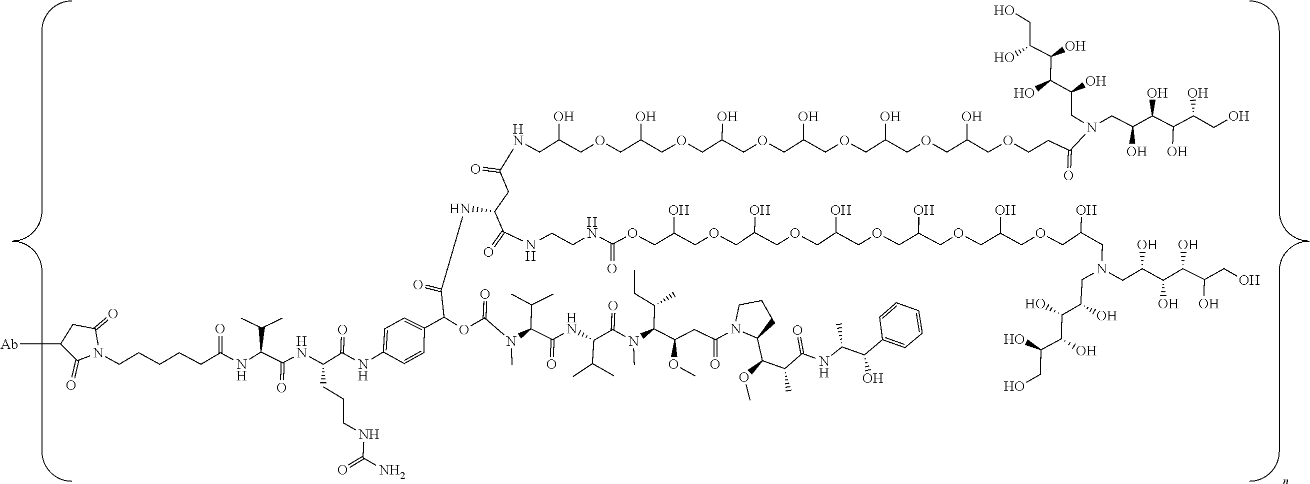 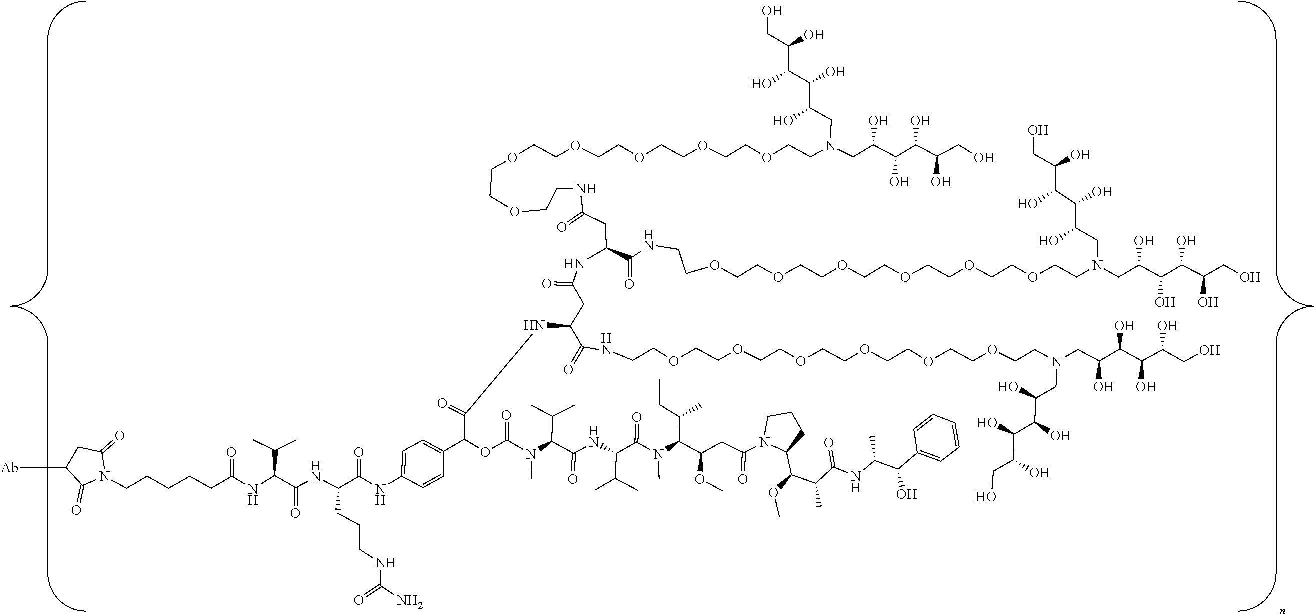 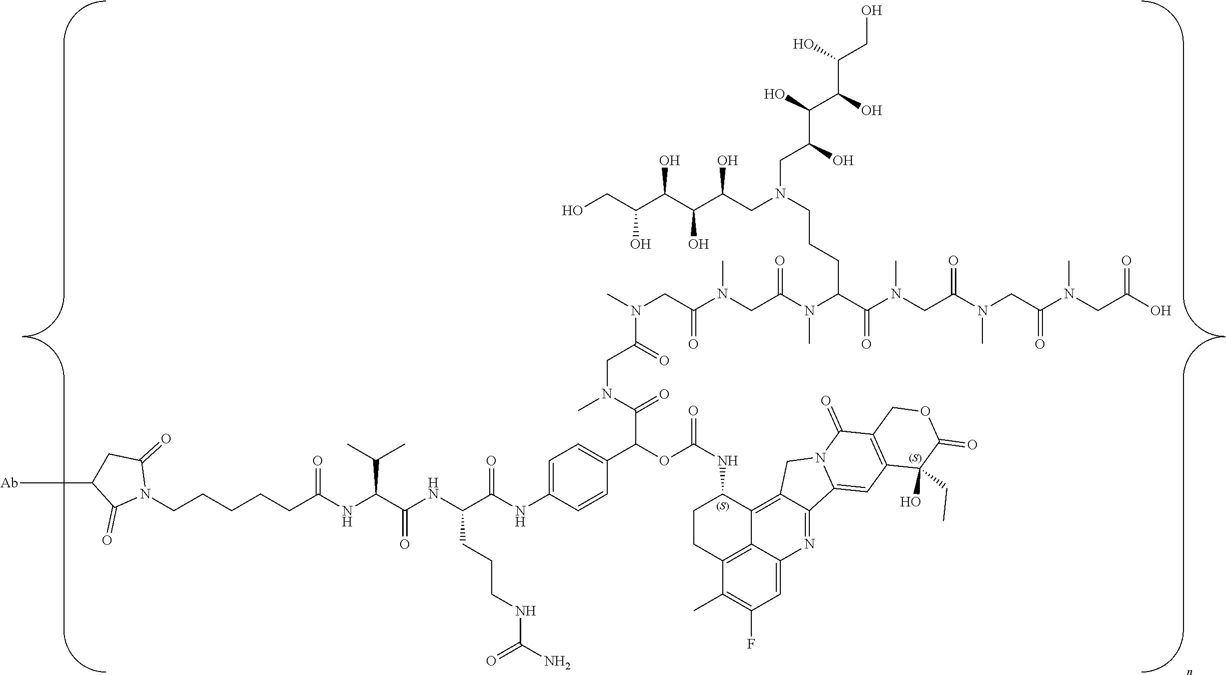 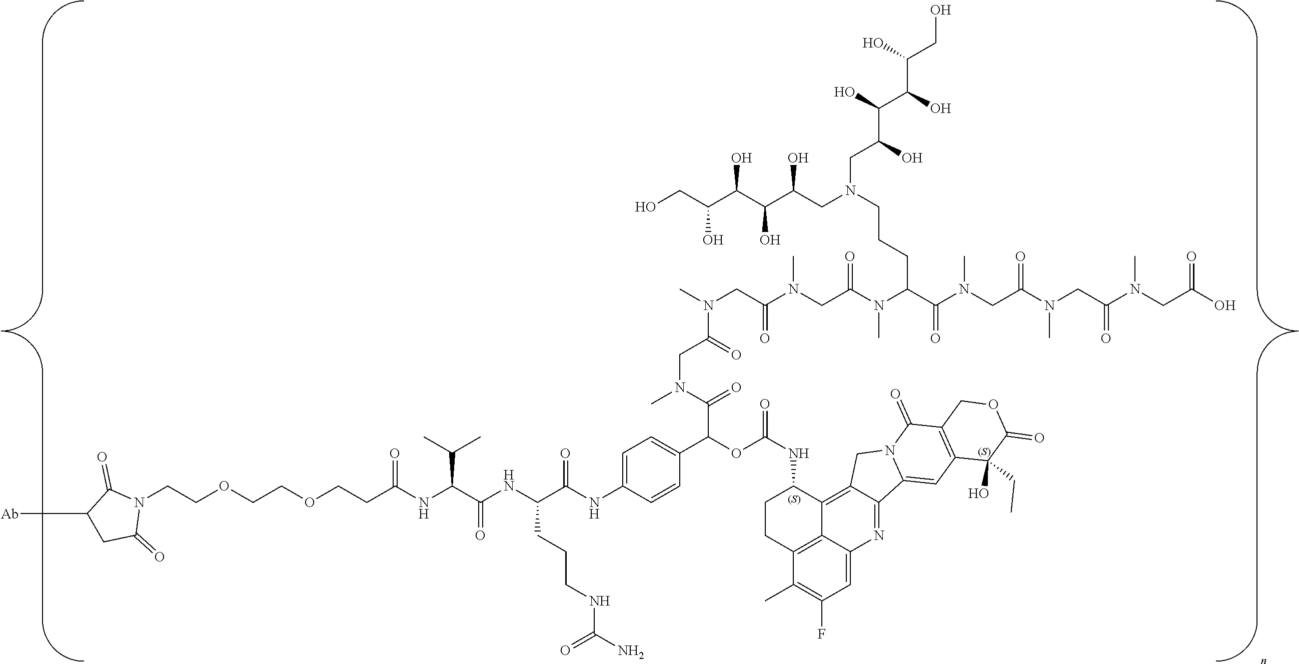 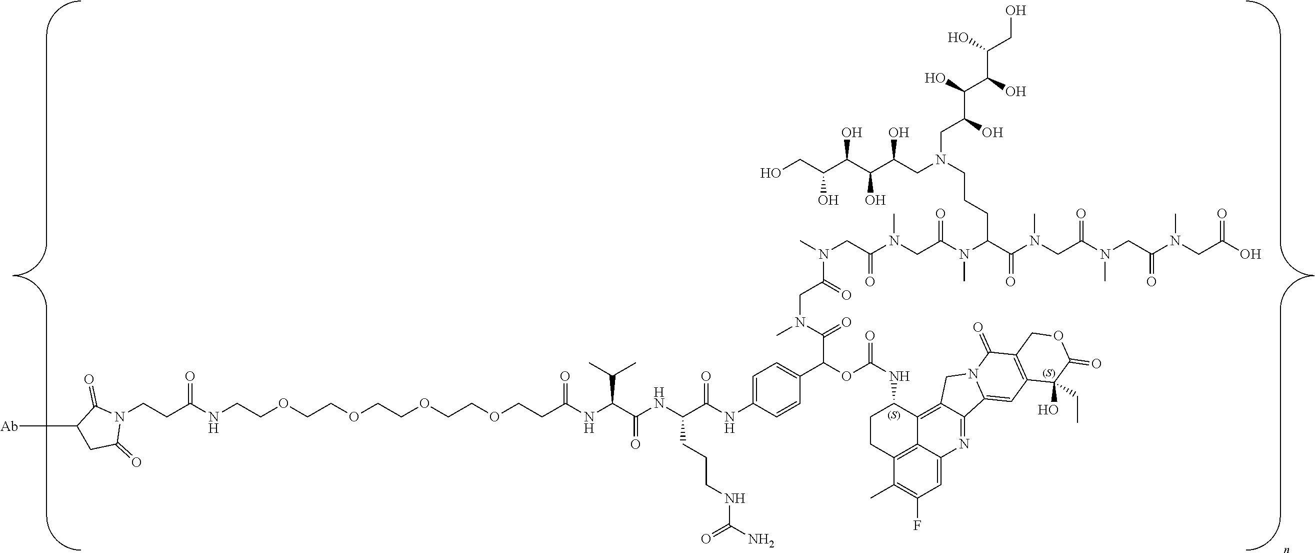 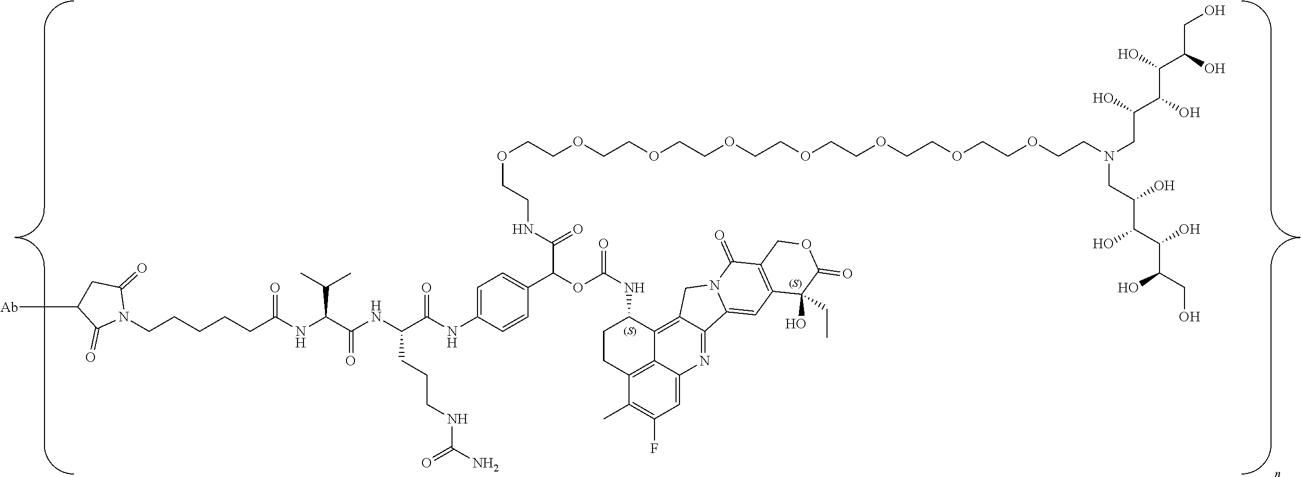 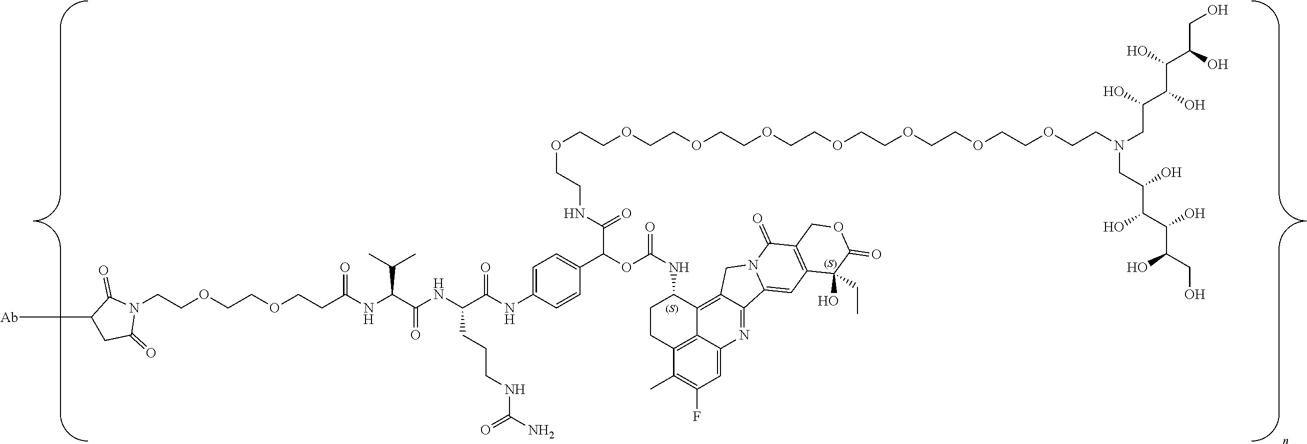 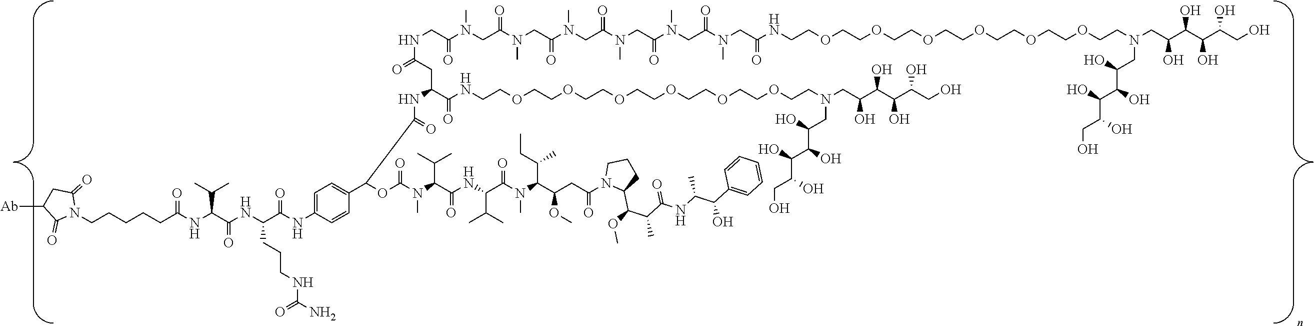 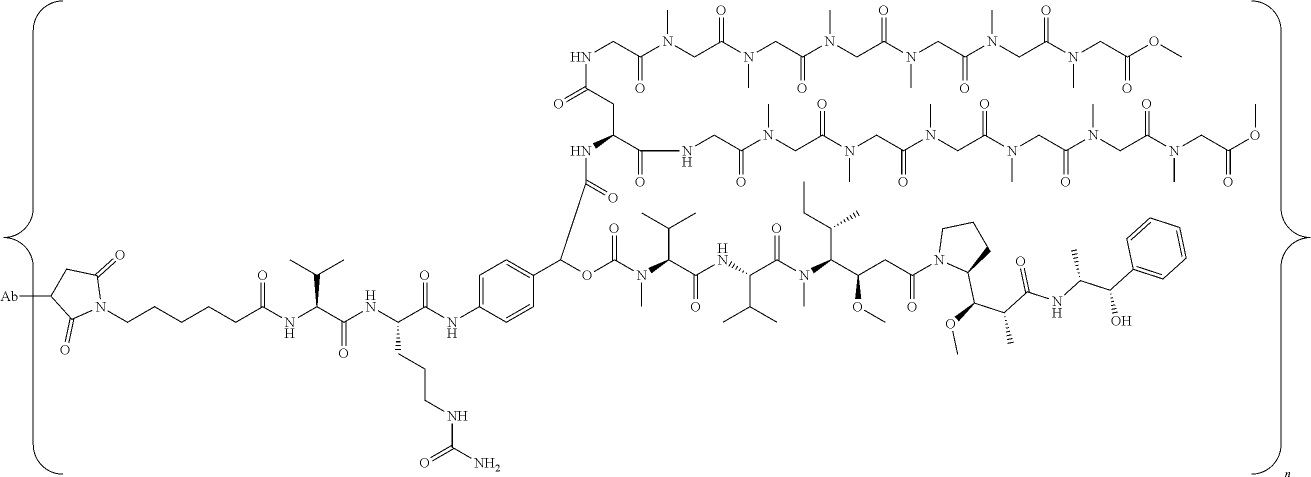 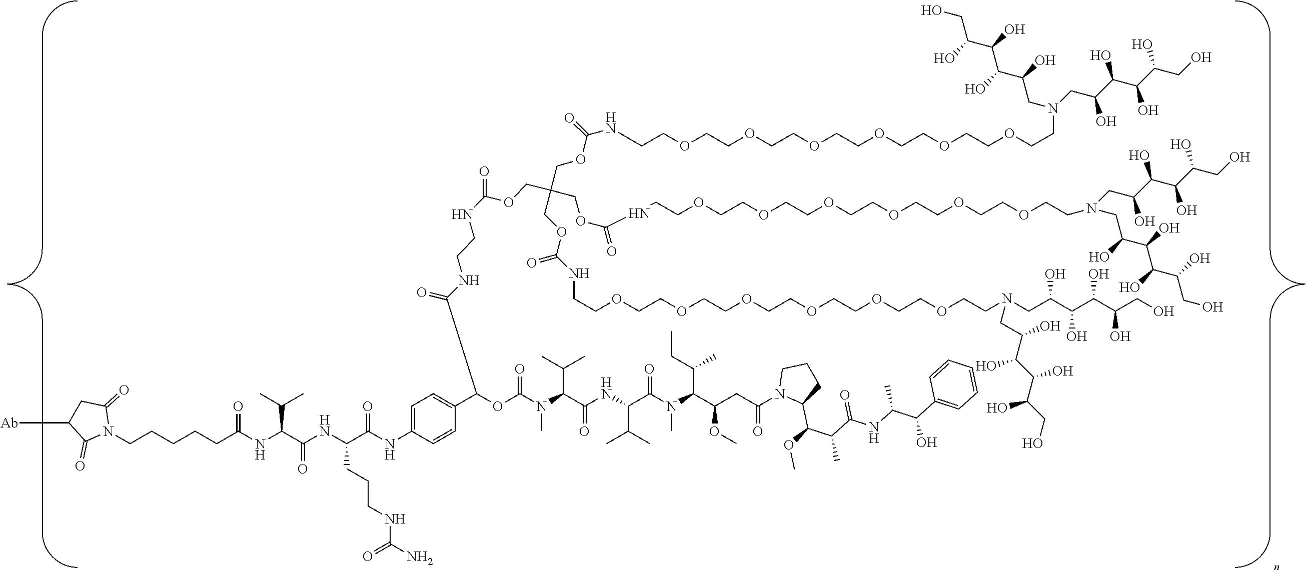 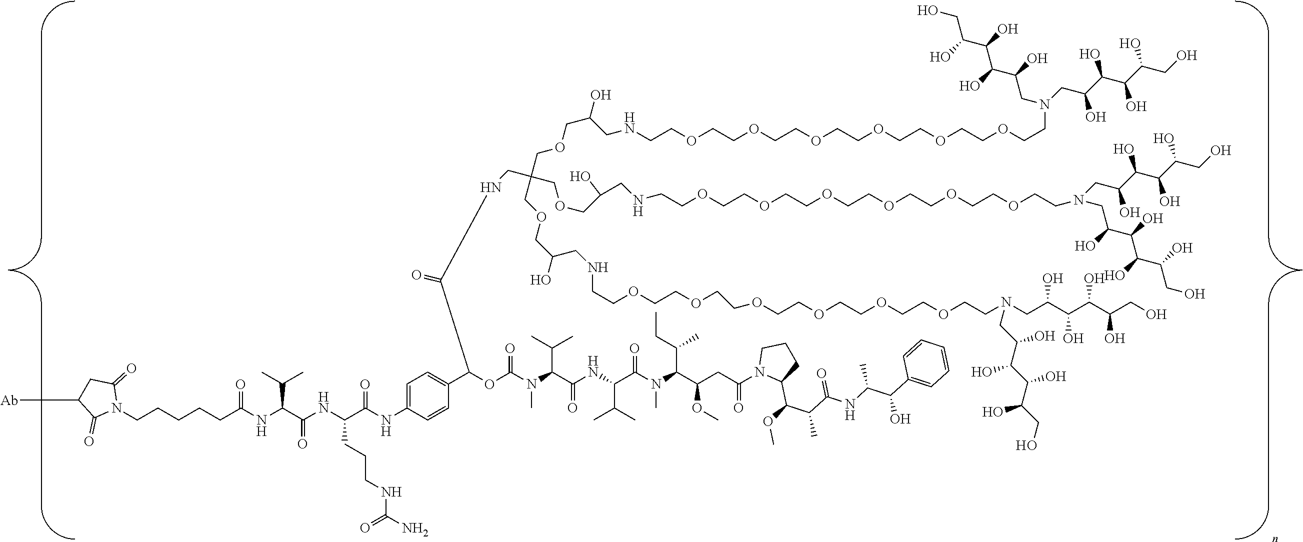 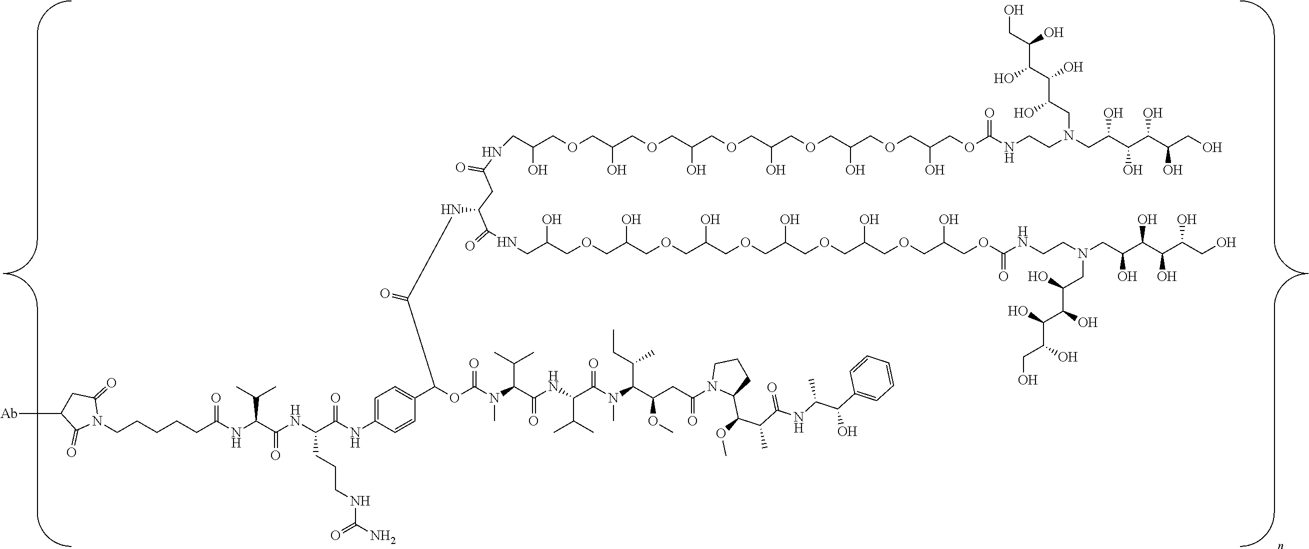 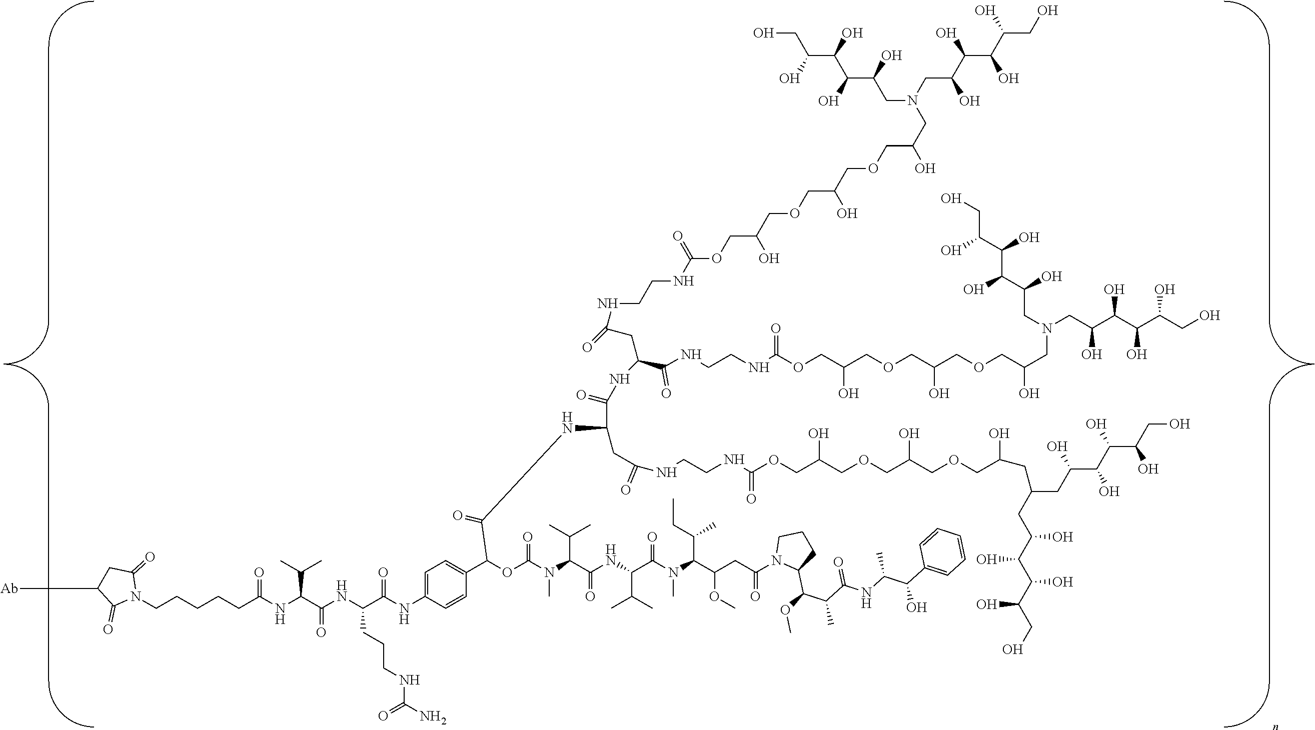 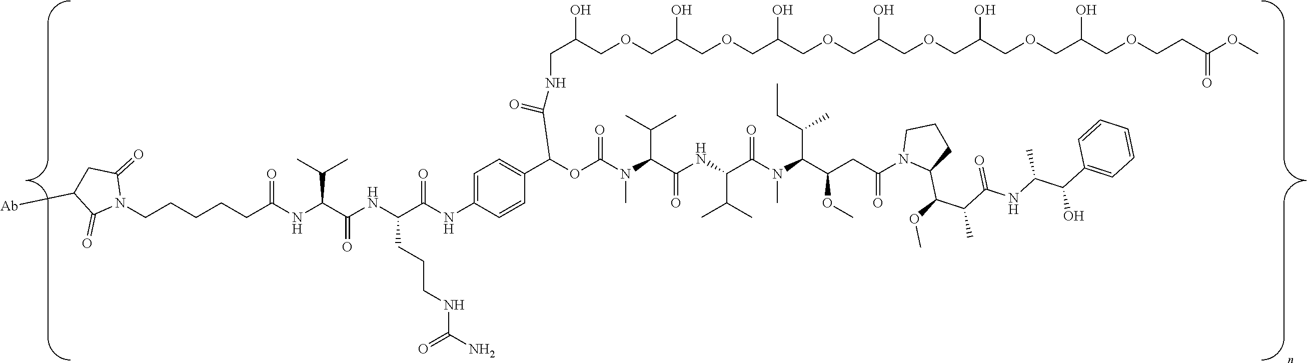 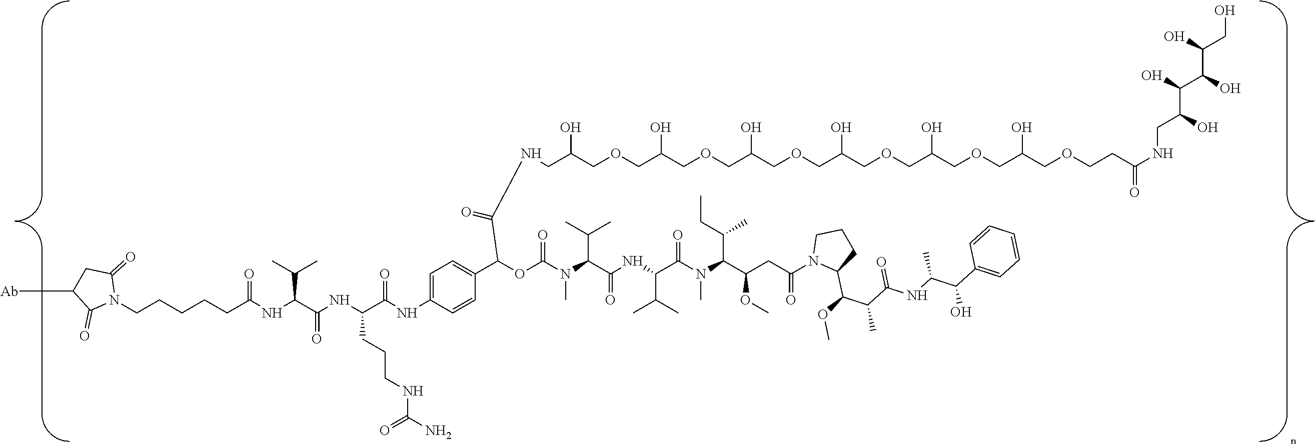 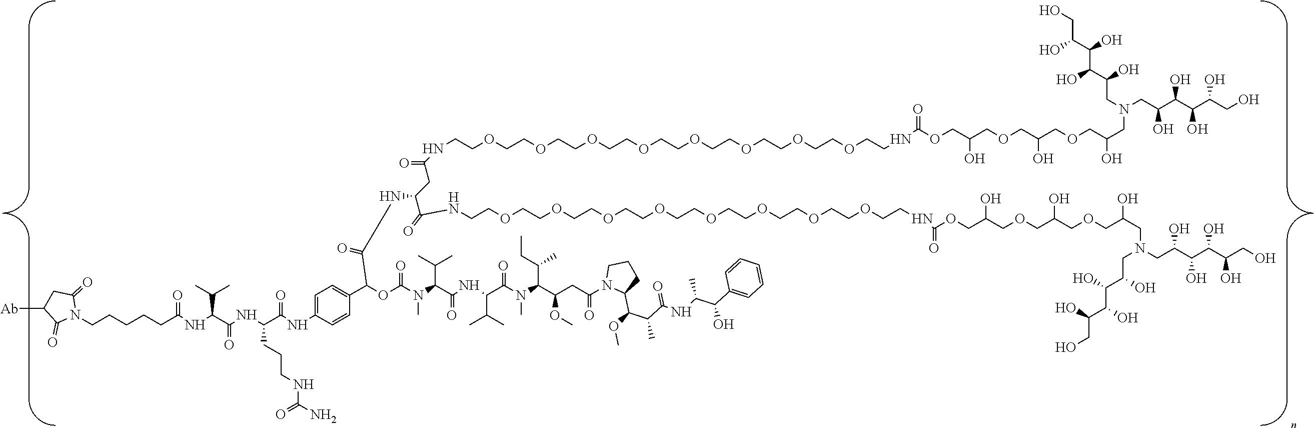 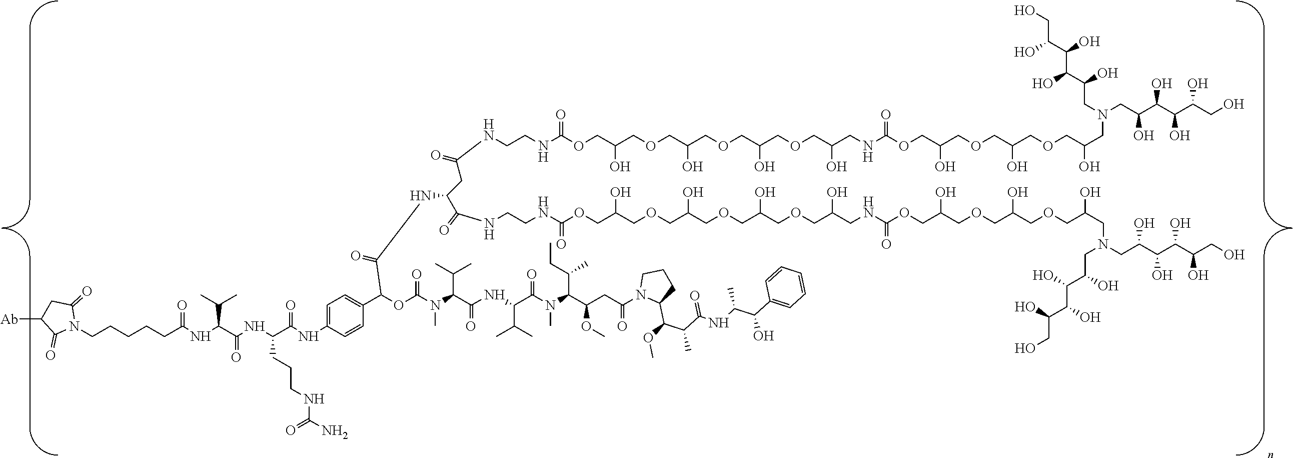 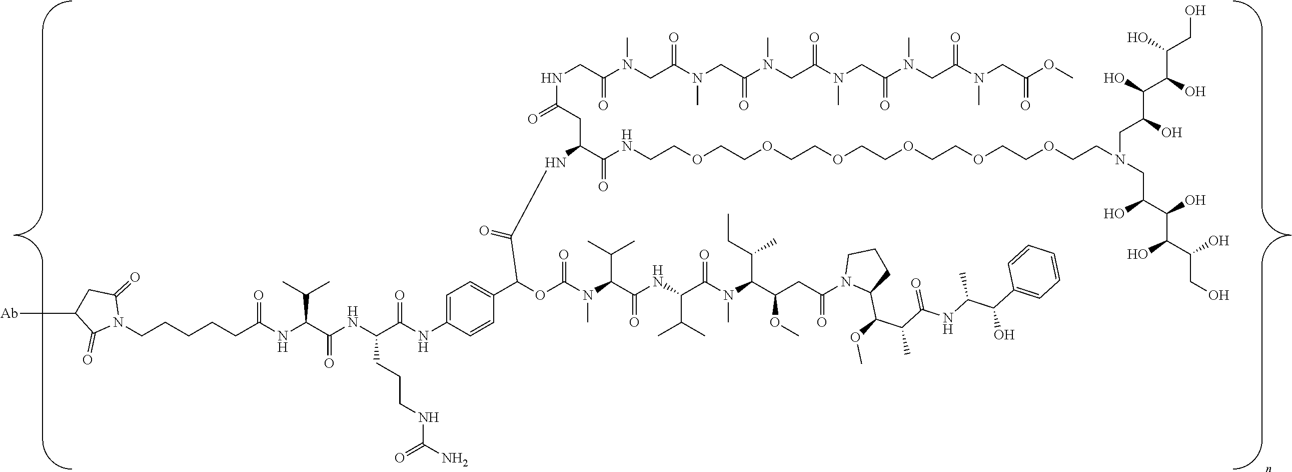 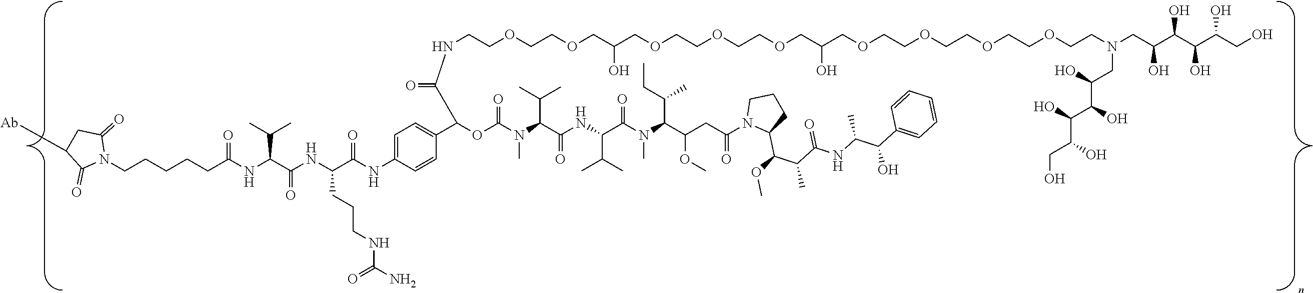 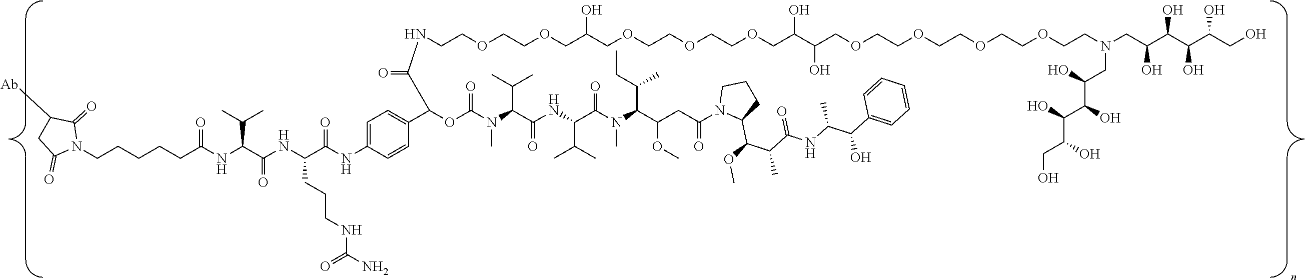 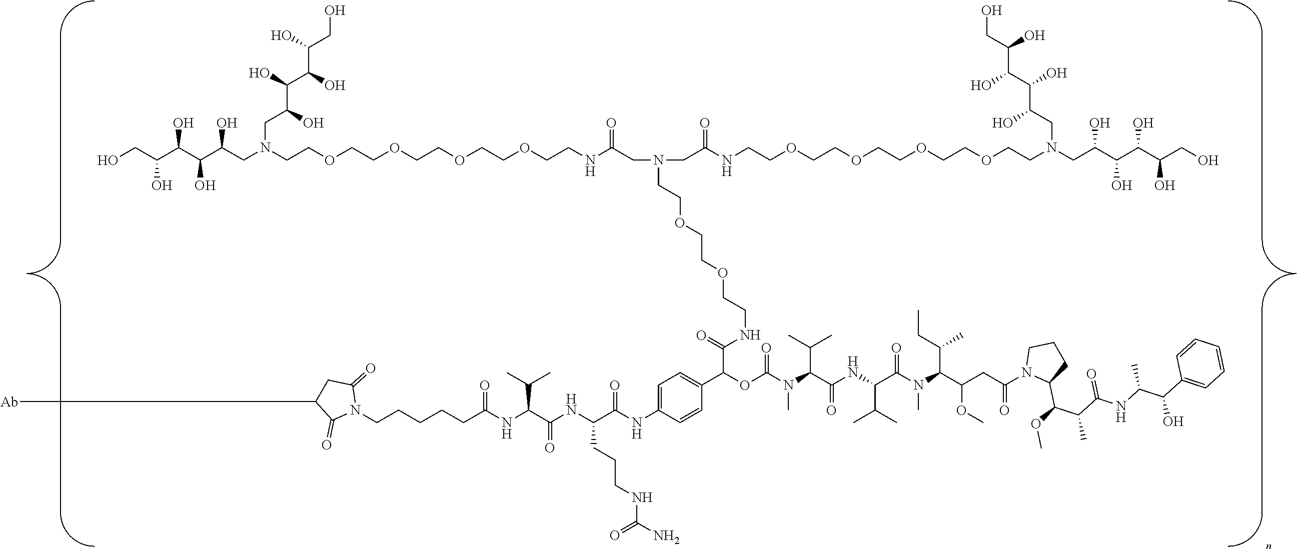 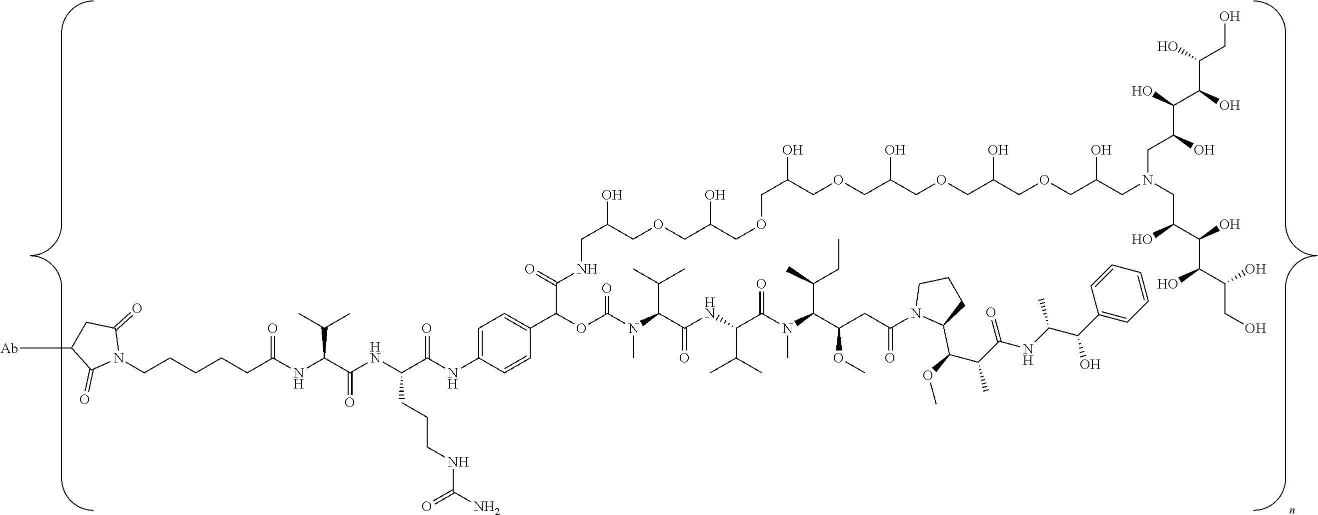 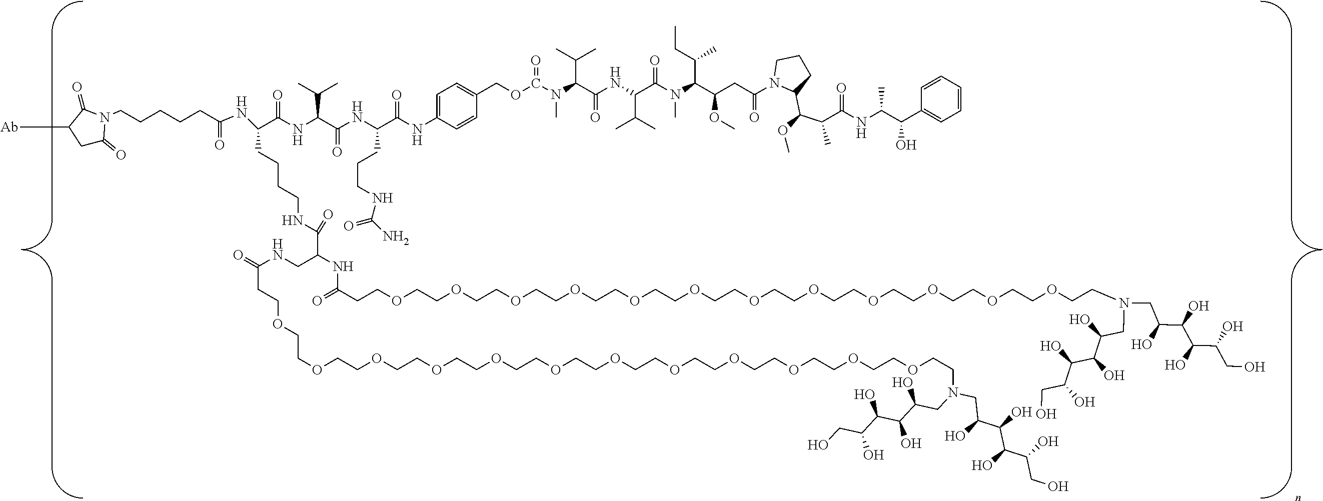 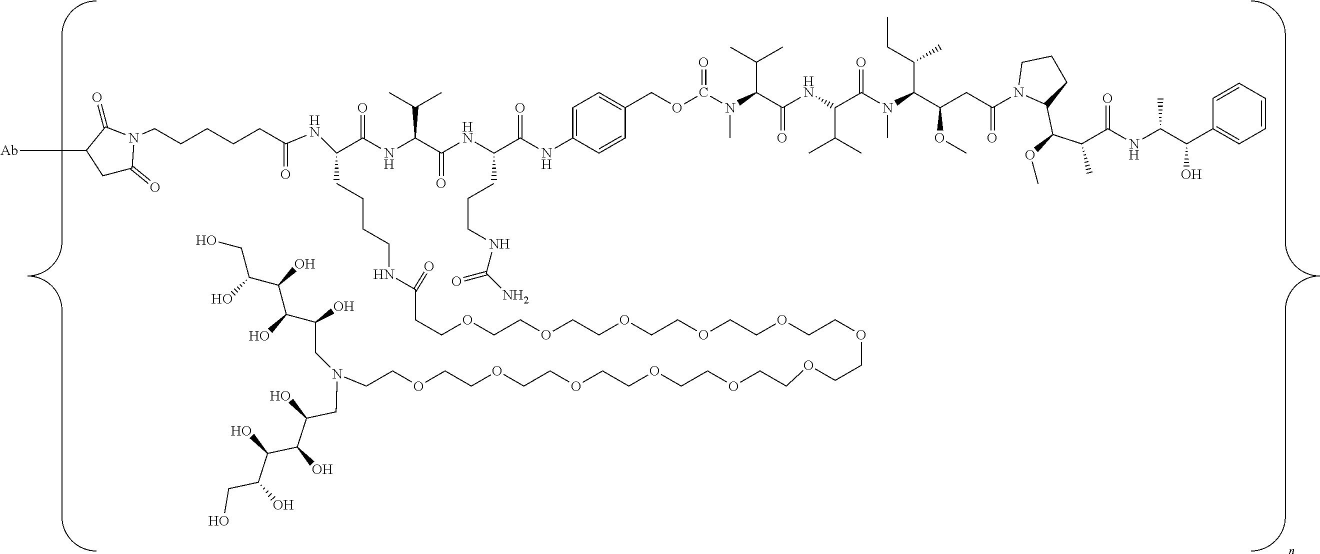 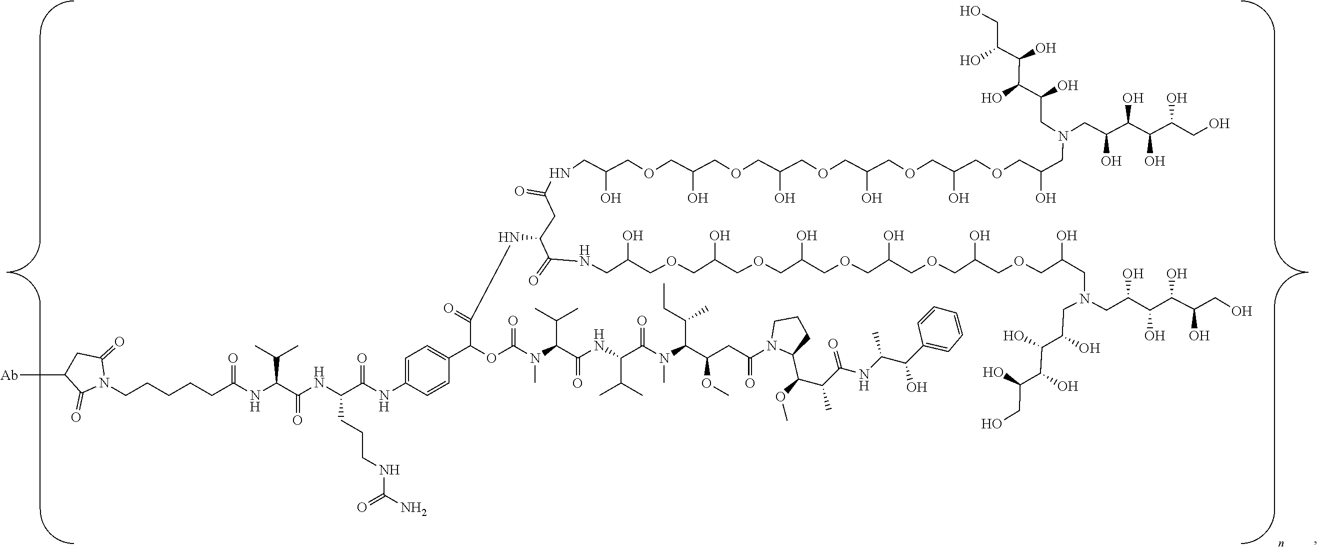 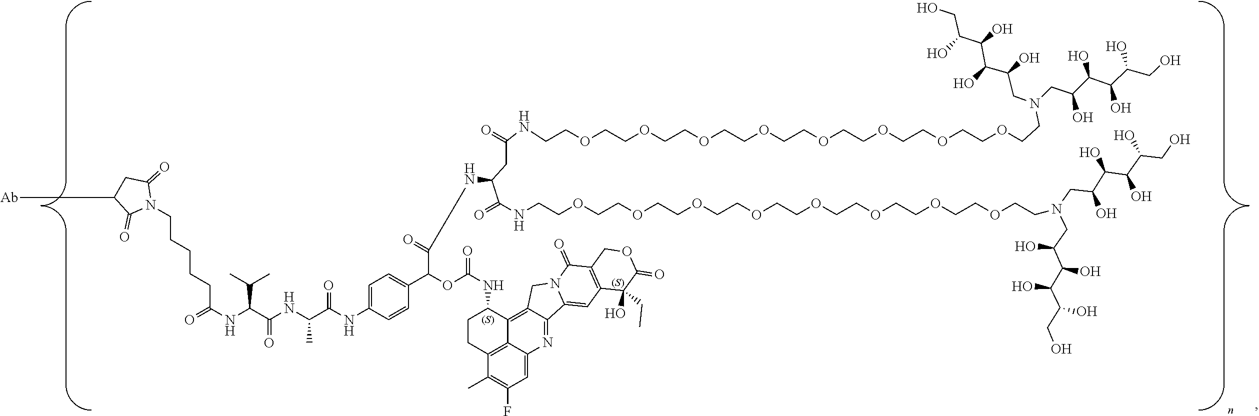 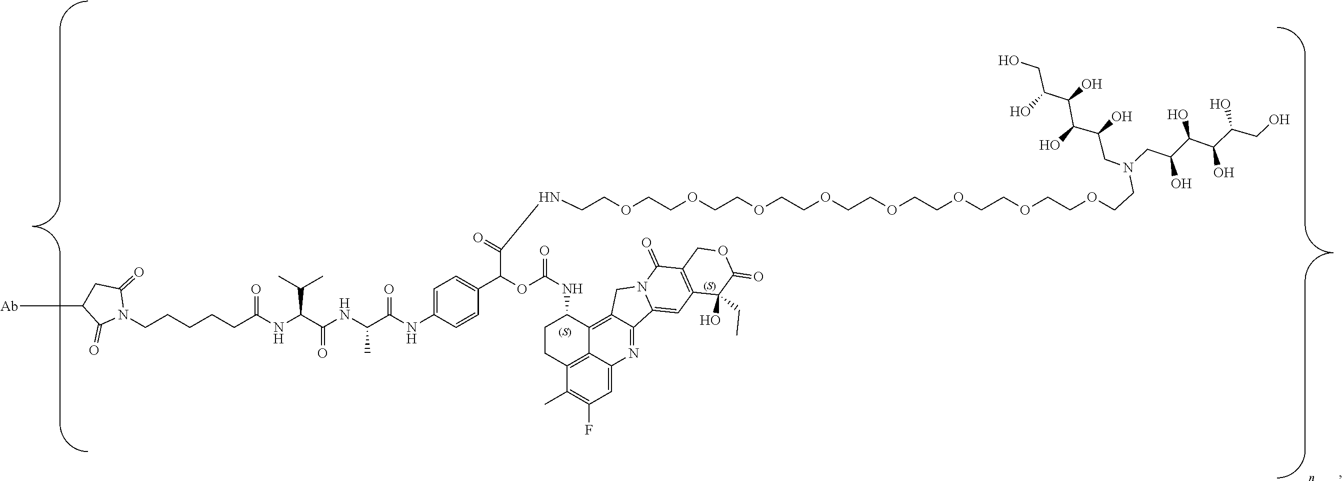 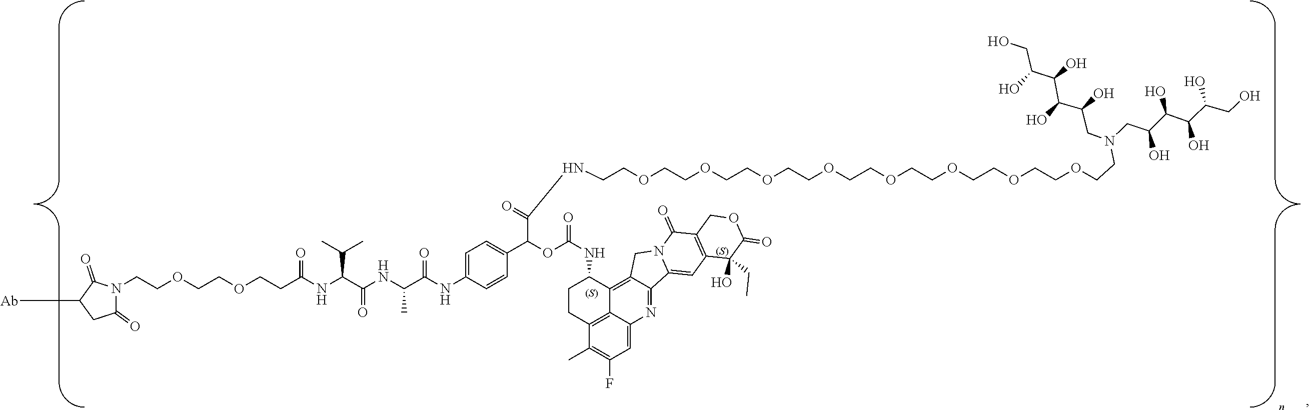 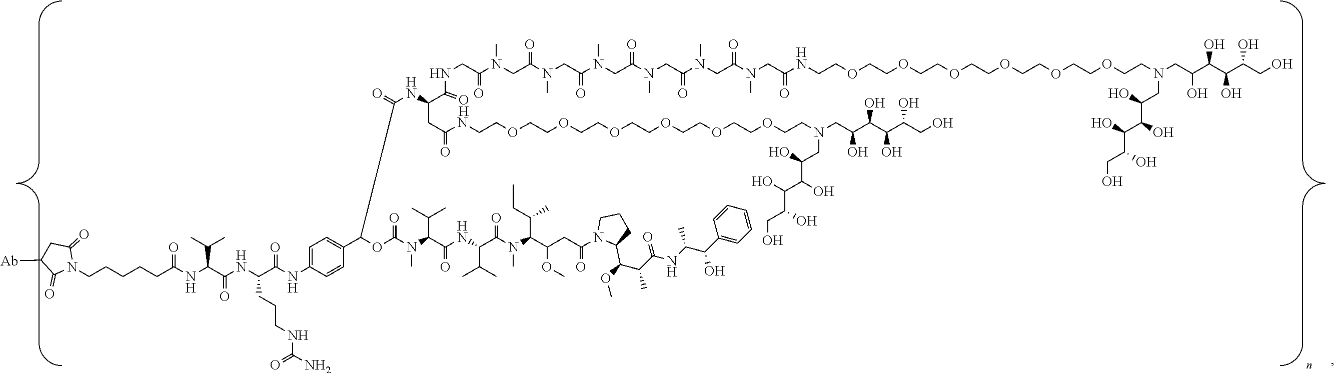 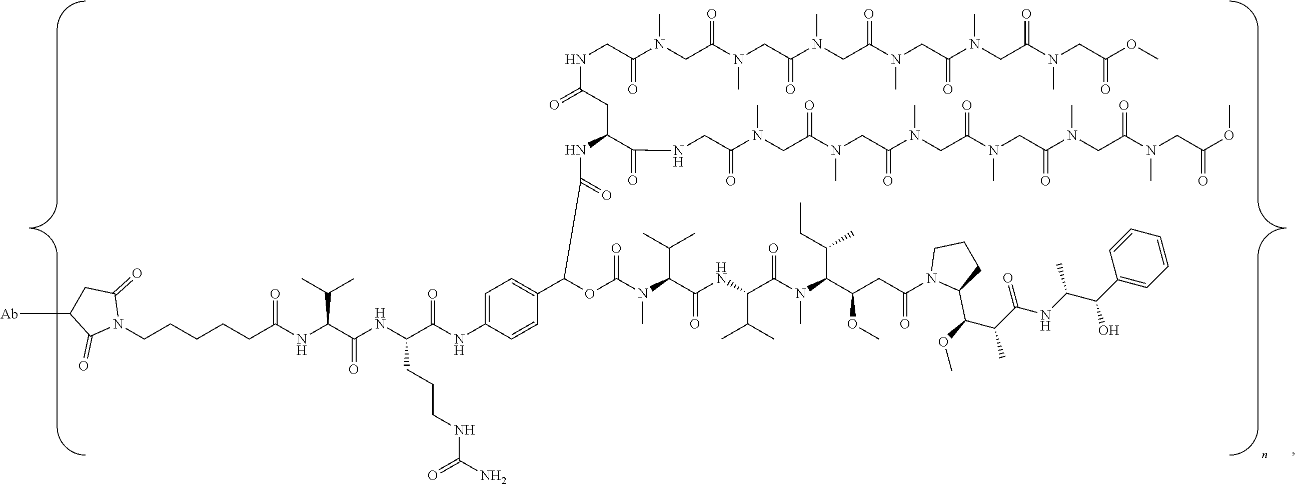 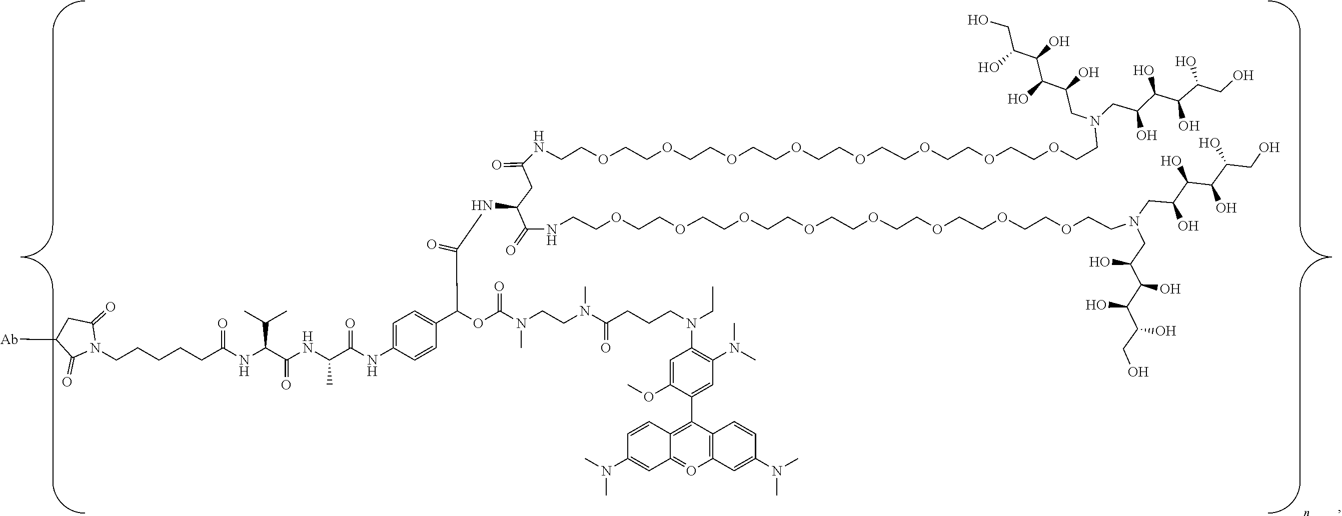 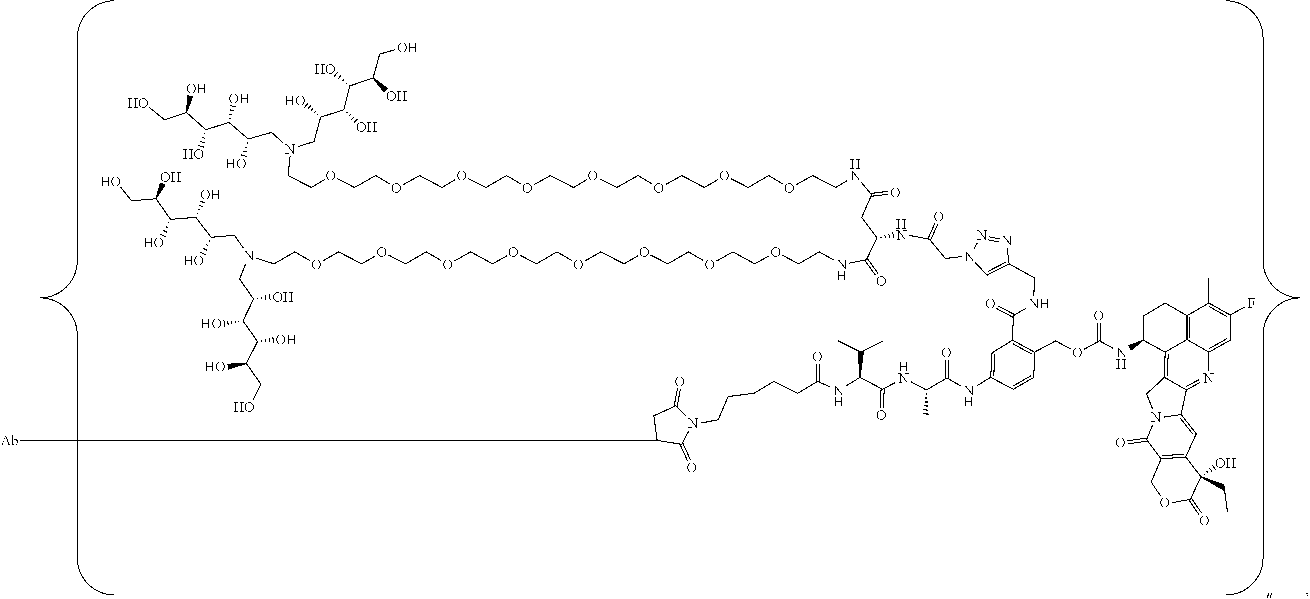 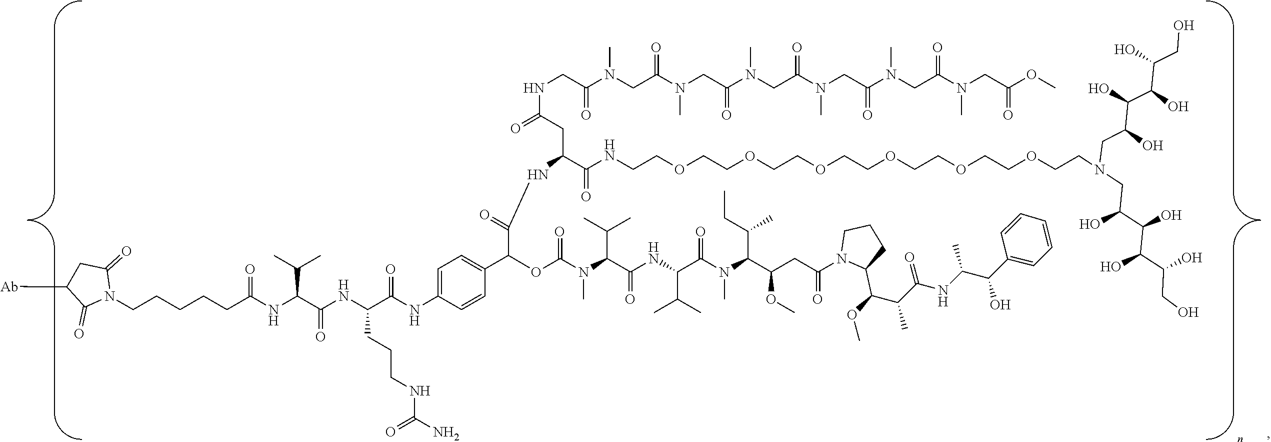
or a stereoisomer thereof, wherein Ab is the bispecific binding agent and n is p^{load}.

56. The conjugate of claim 23, wherein the conjugate has following structure: 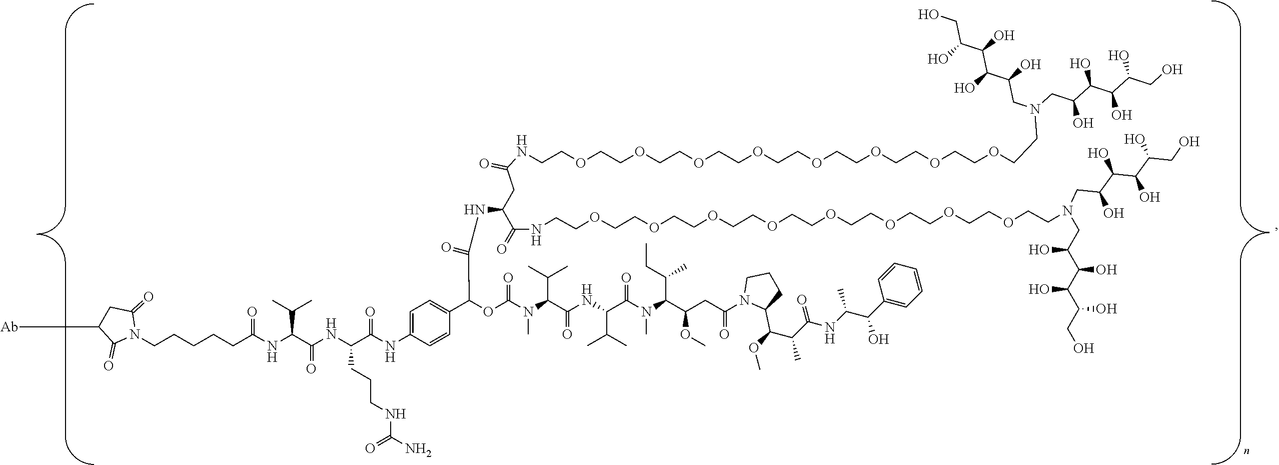
or a stereoisomer thereof, wherein Ab is the bispecific binding agent and n is p^{load}, wherein p^{load} is from about 1 to about 8, about 2, about 4, about 6, about 8, about 10, about 12, about 14, about 16, about 3 to about 5, about 6 to about 8, or about 8 to about 16.

57. The conjugate of claim 23, selected from the following: 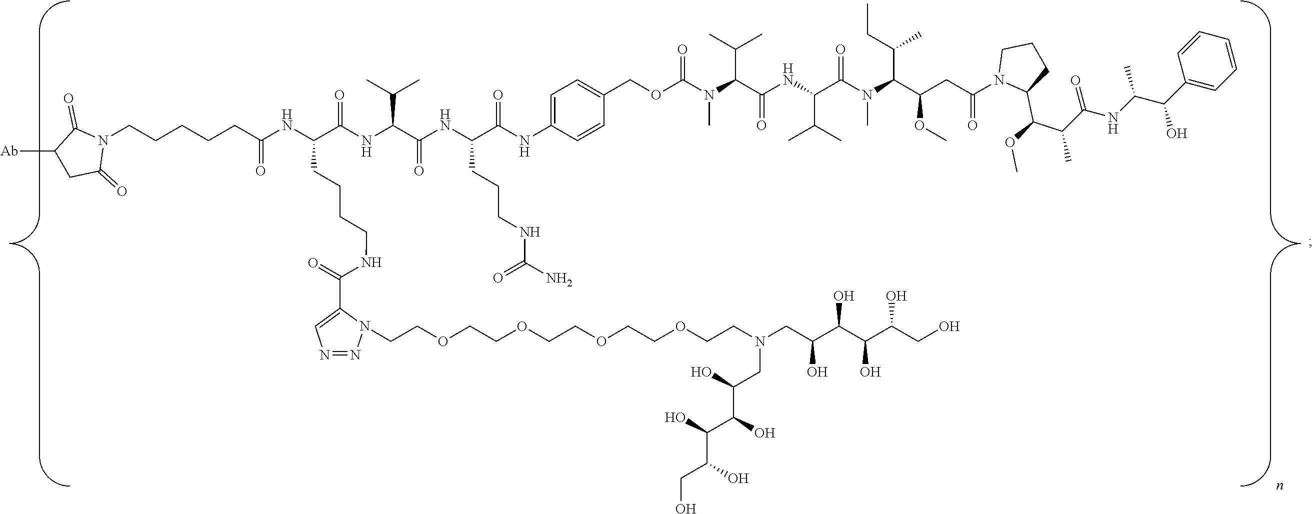 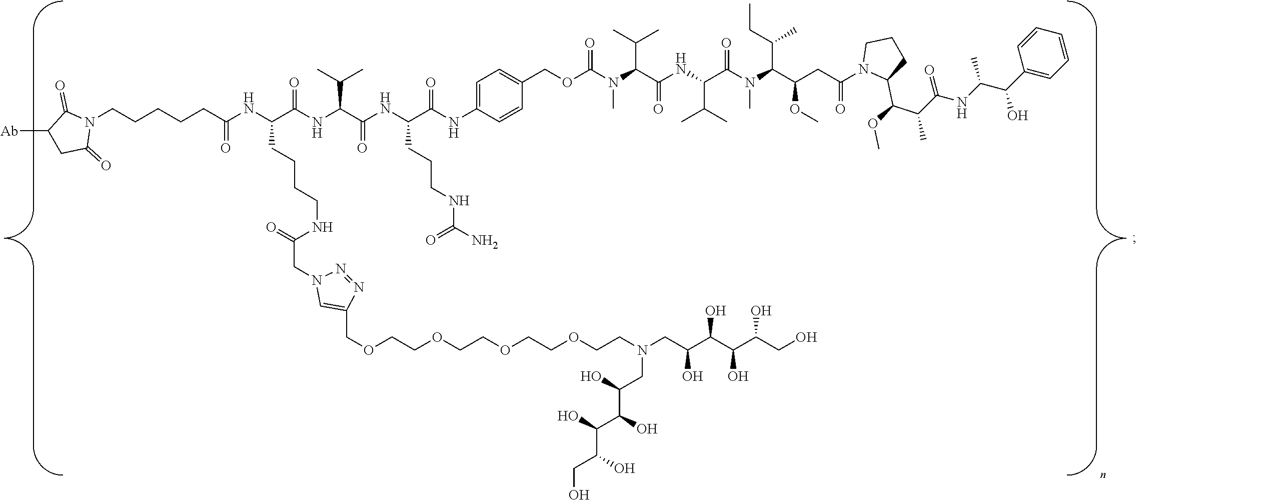 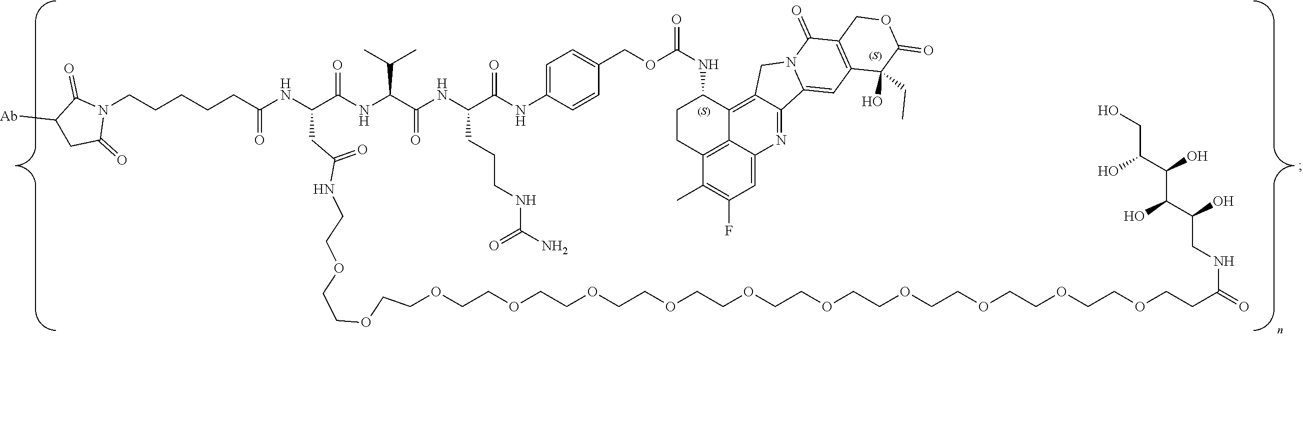 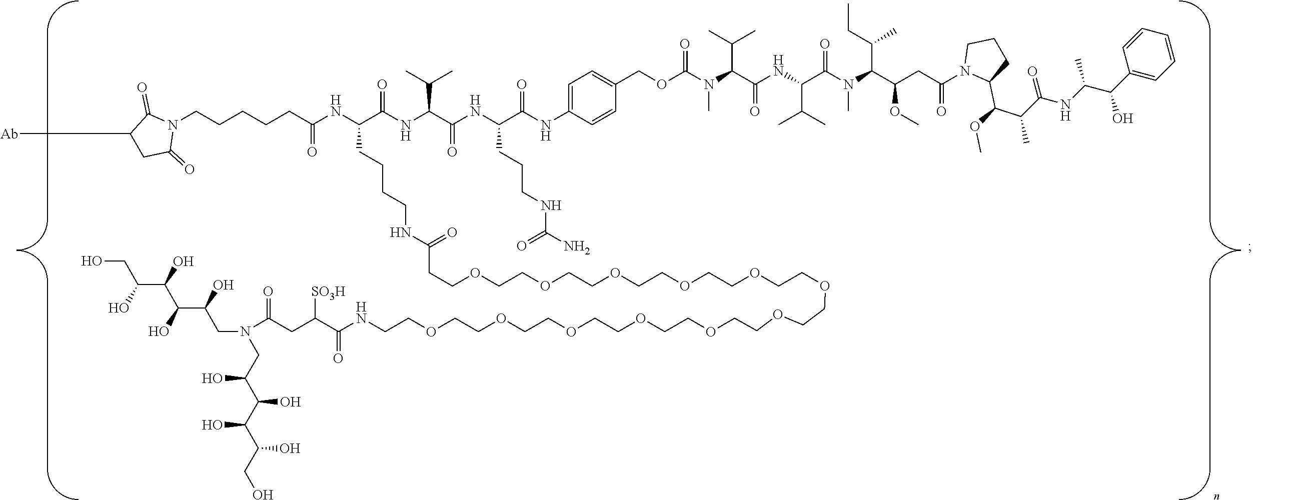 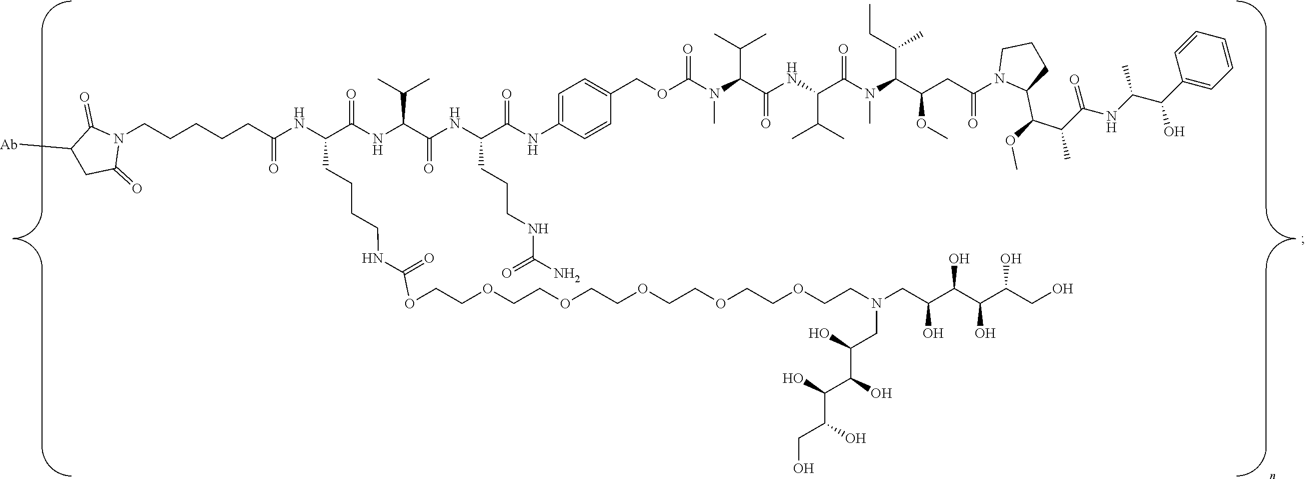 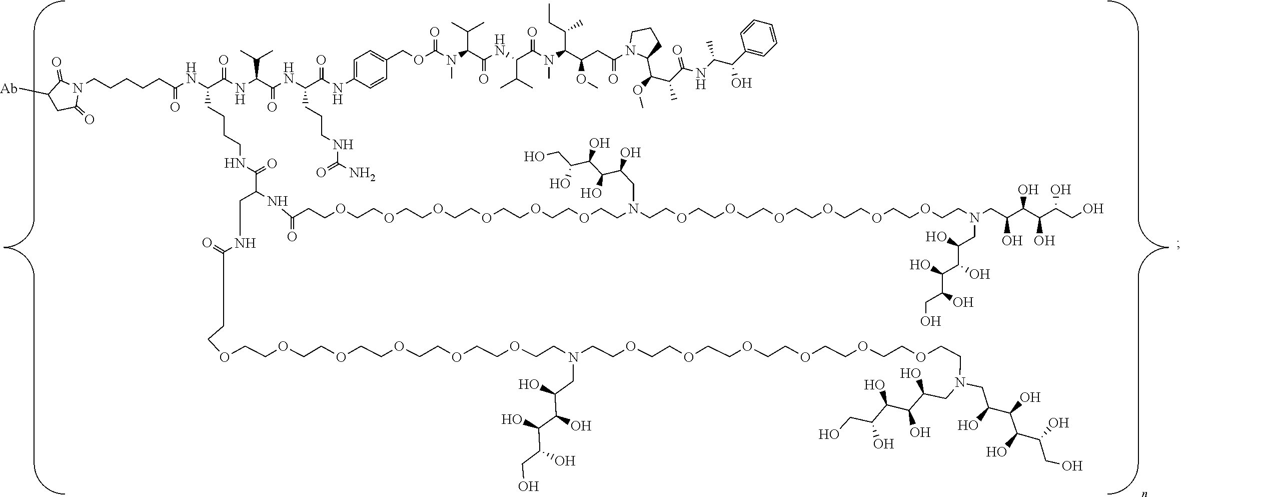 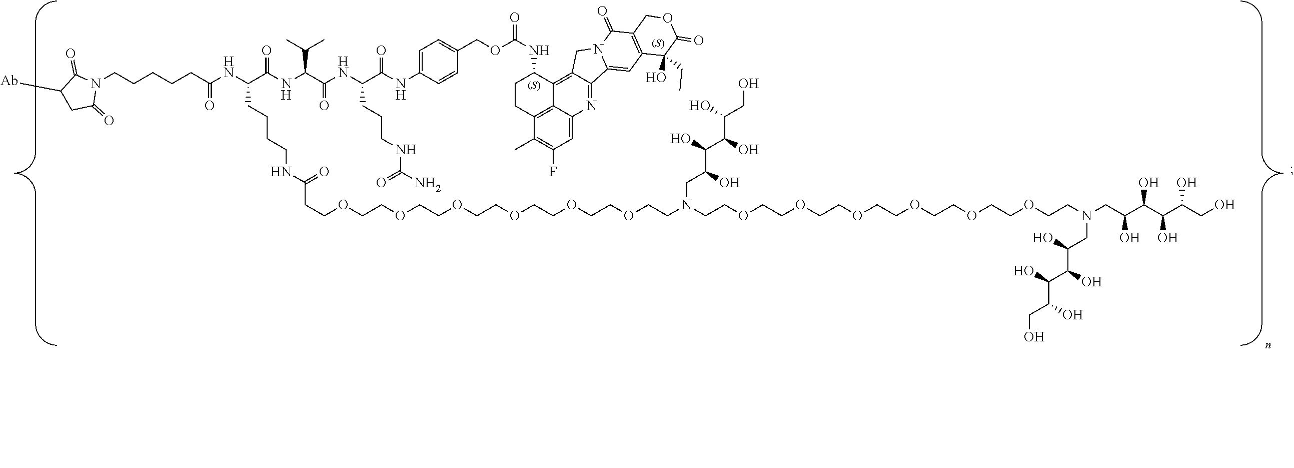 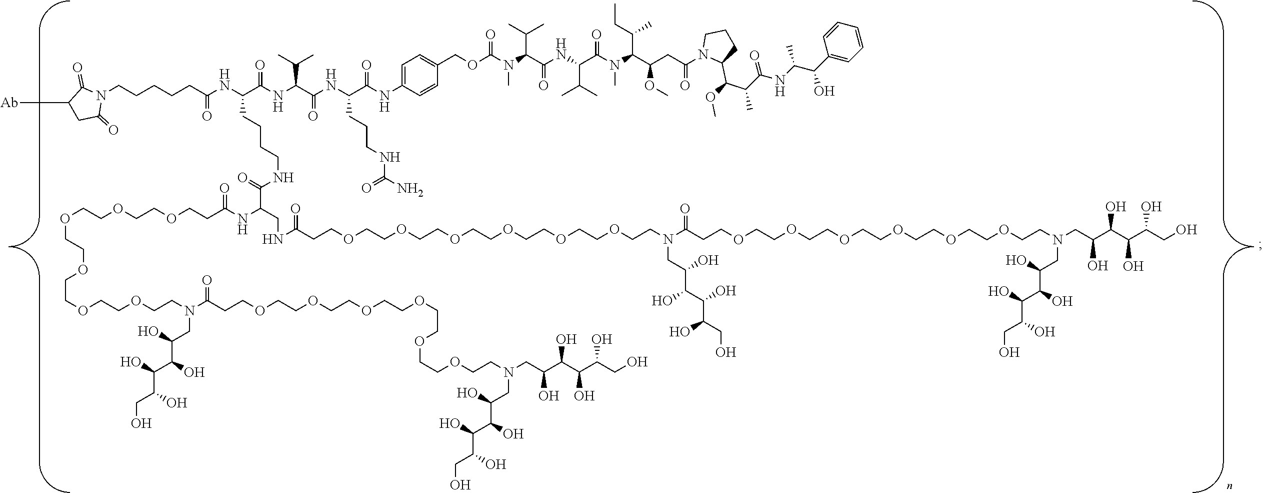 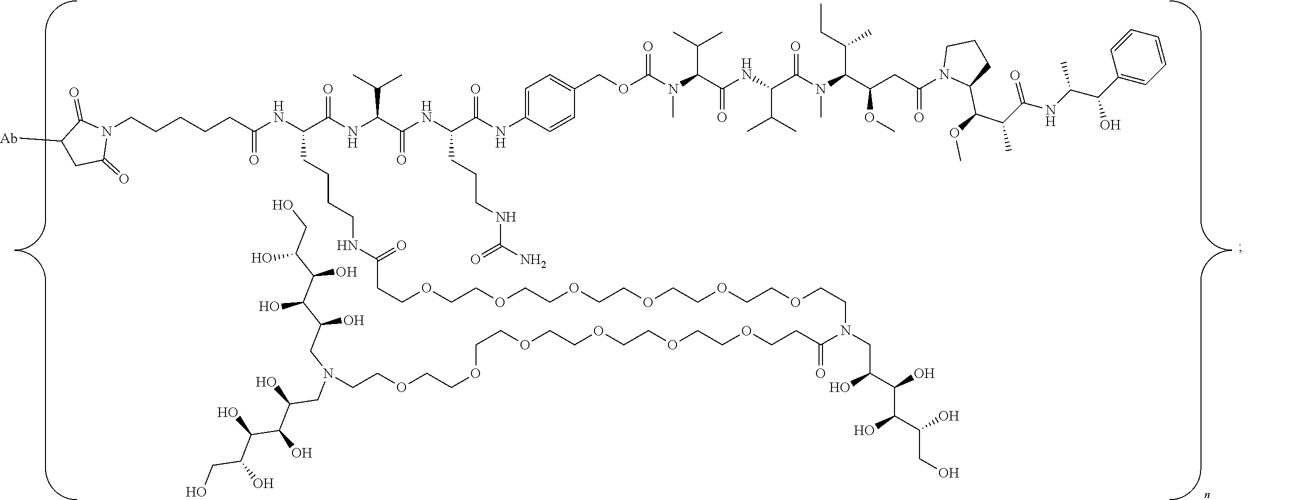 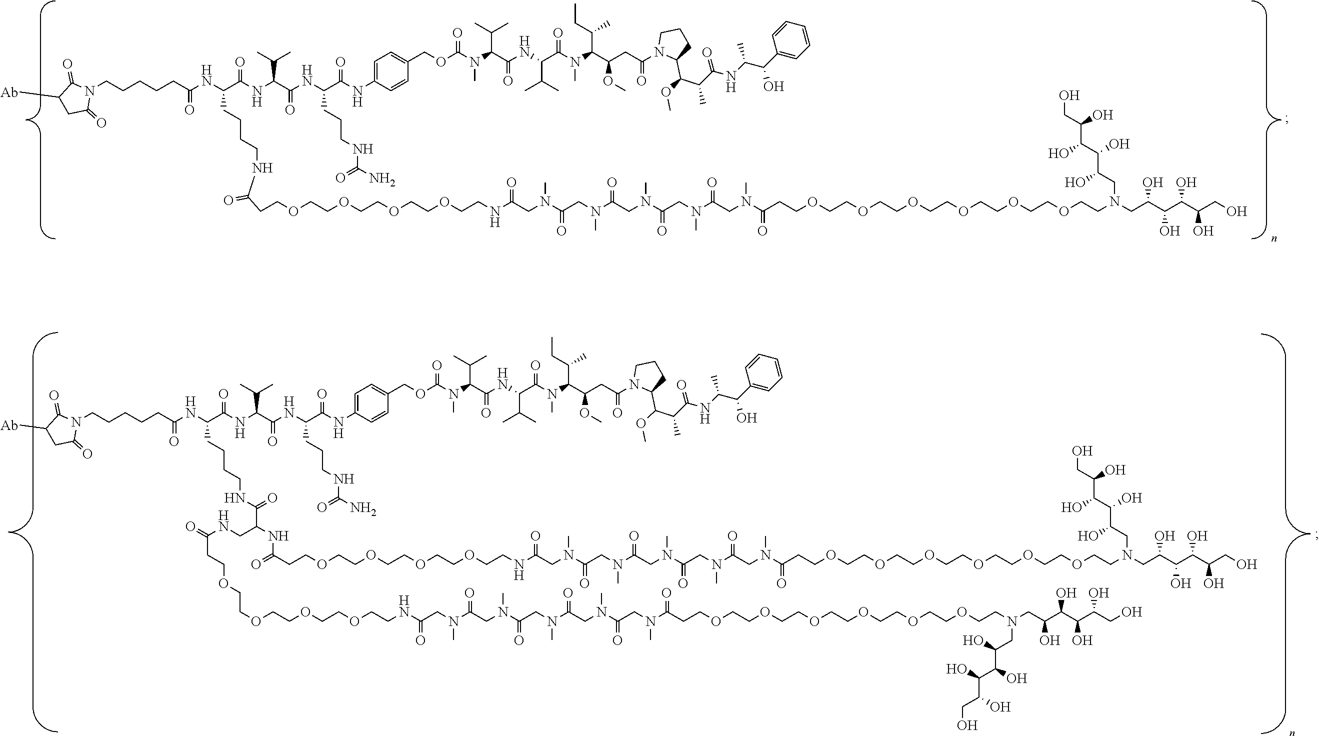 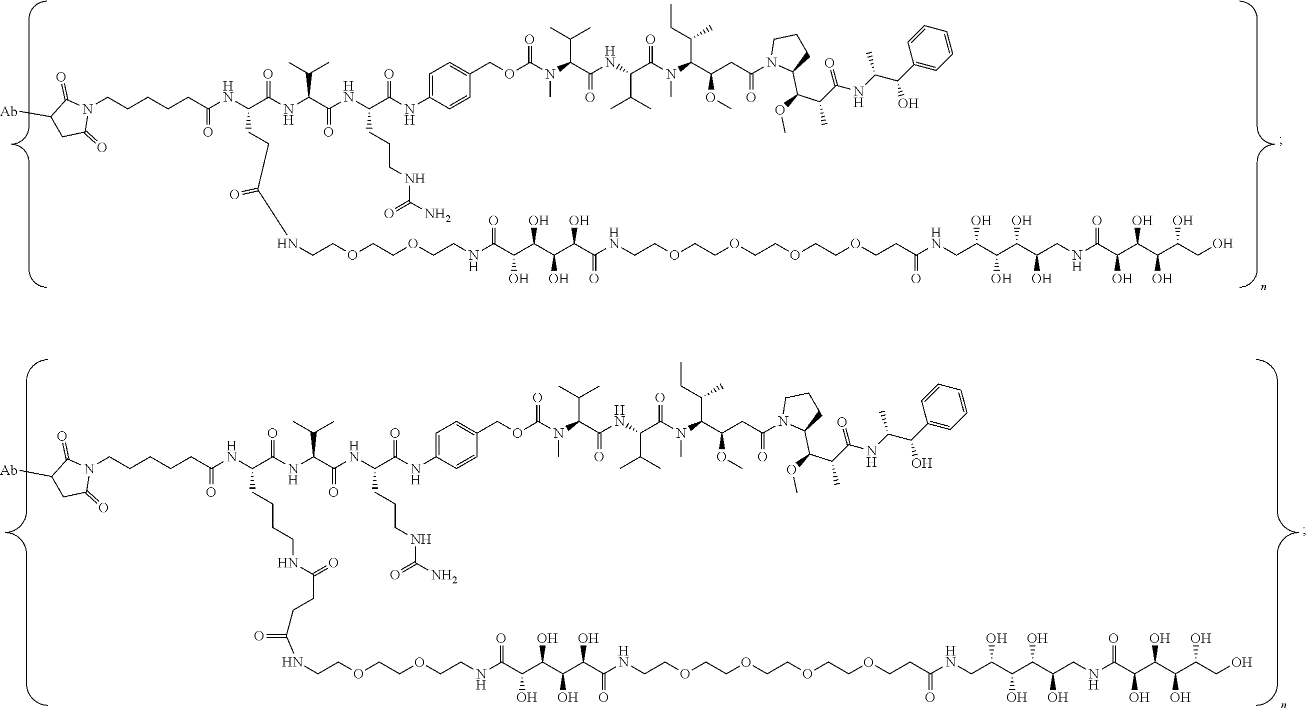 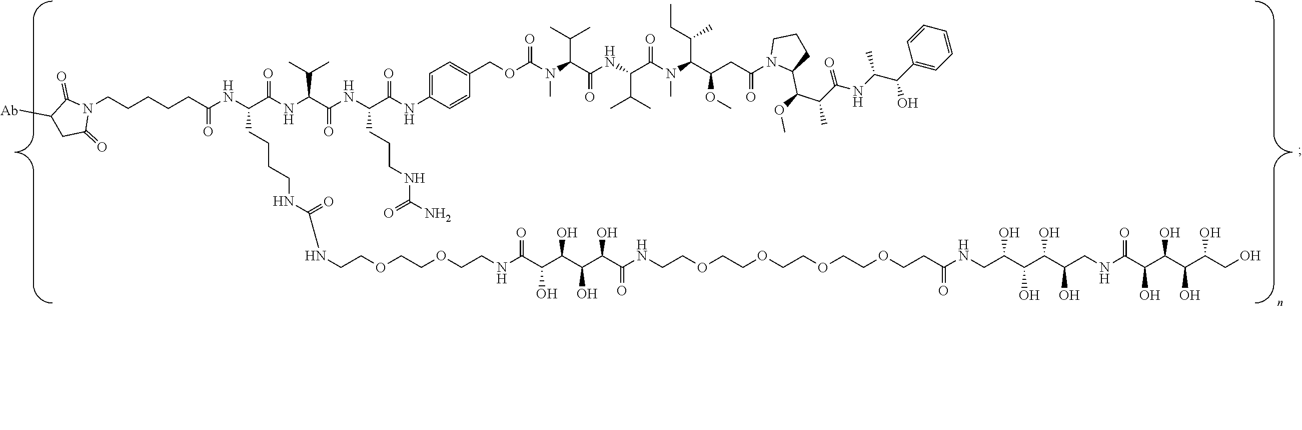 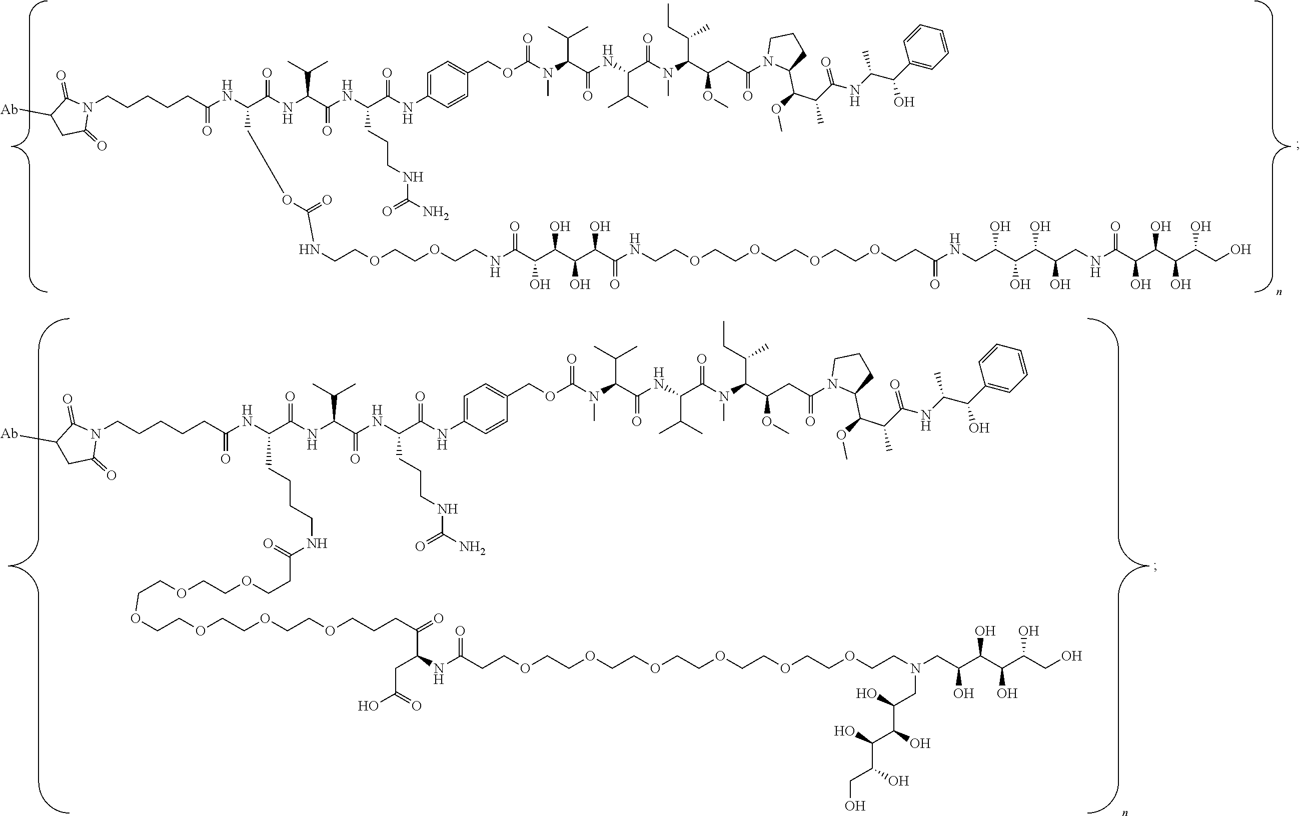 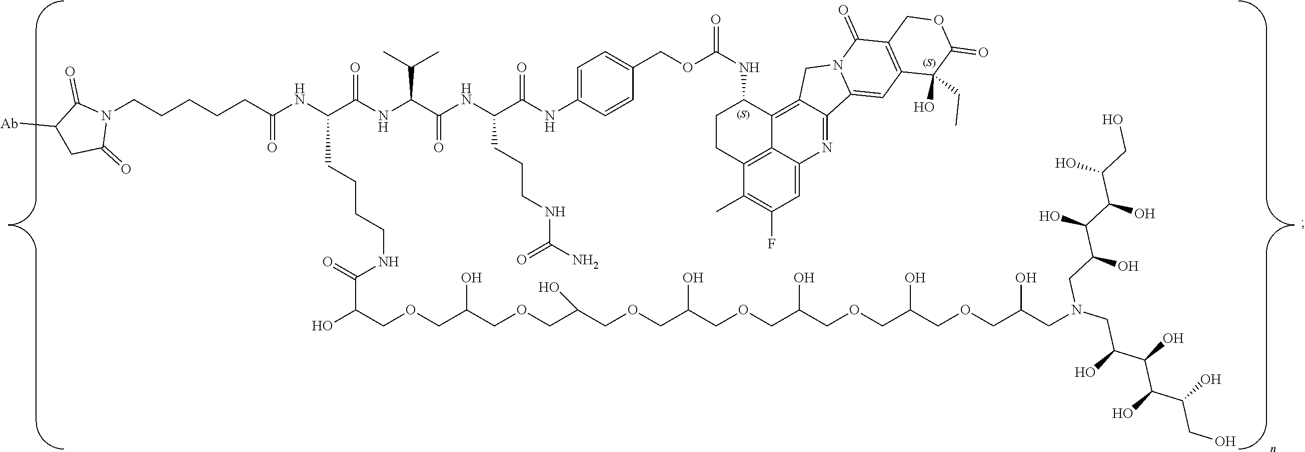 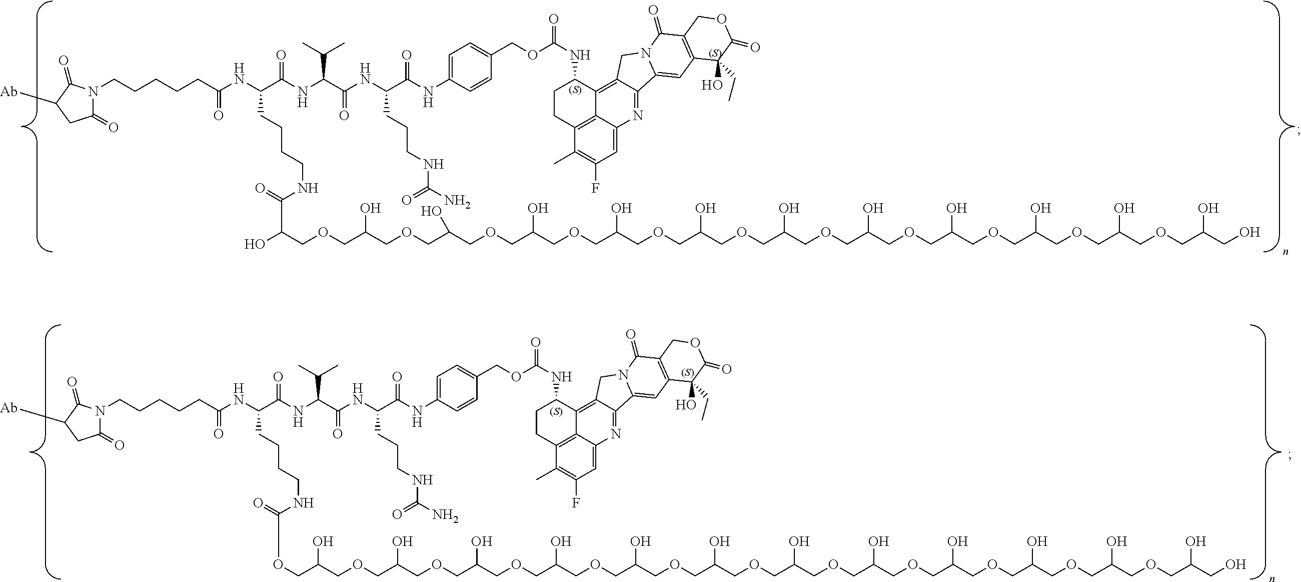 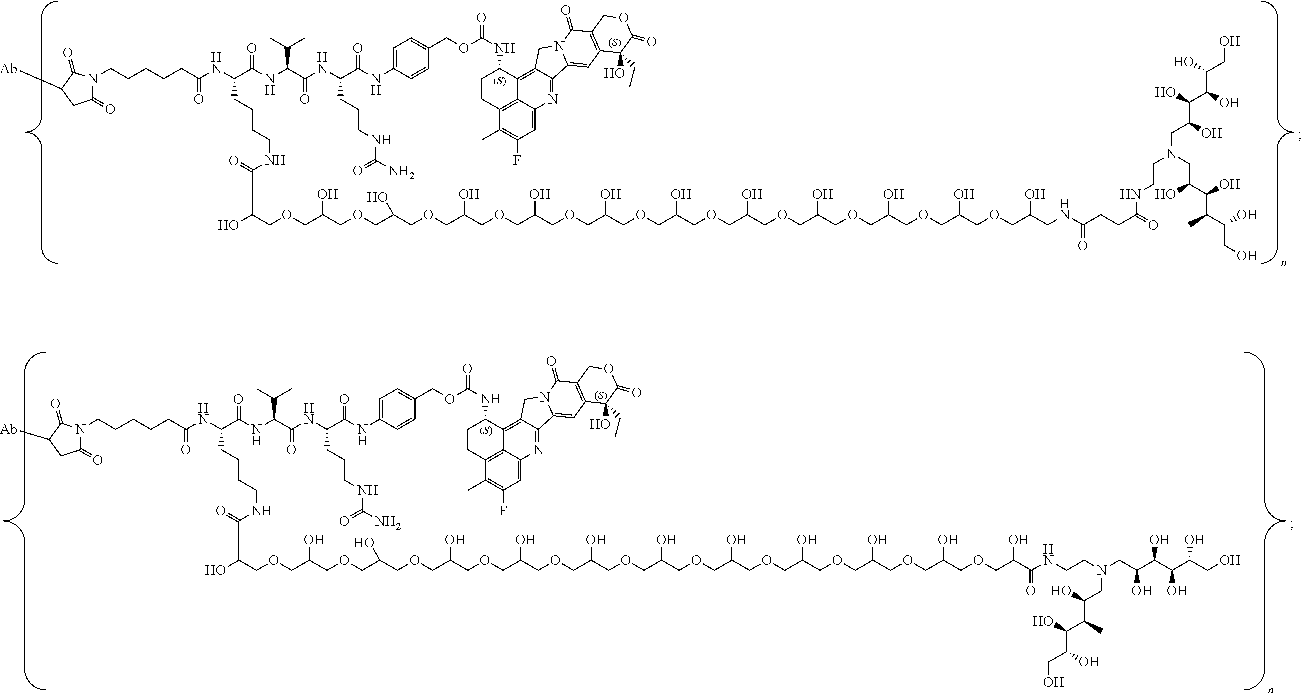 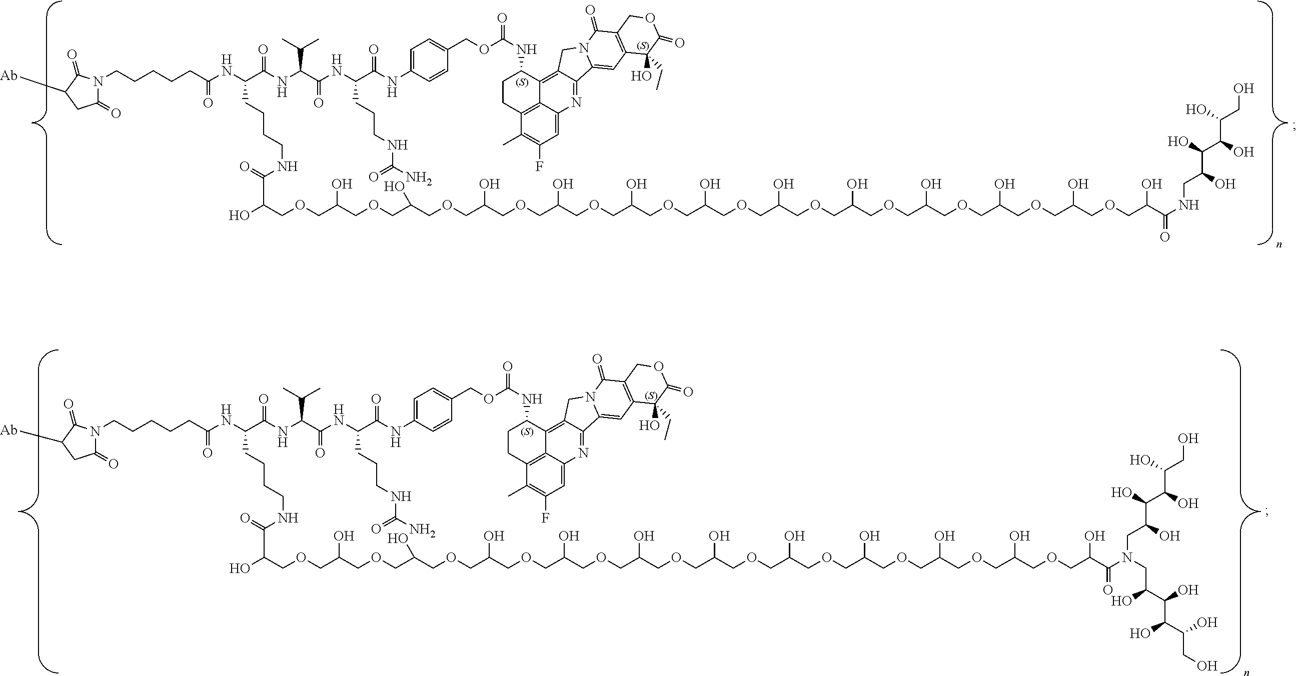 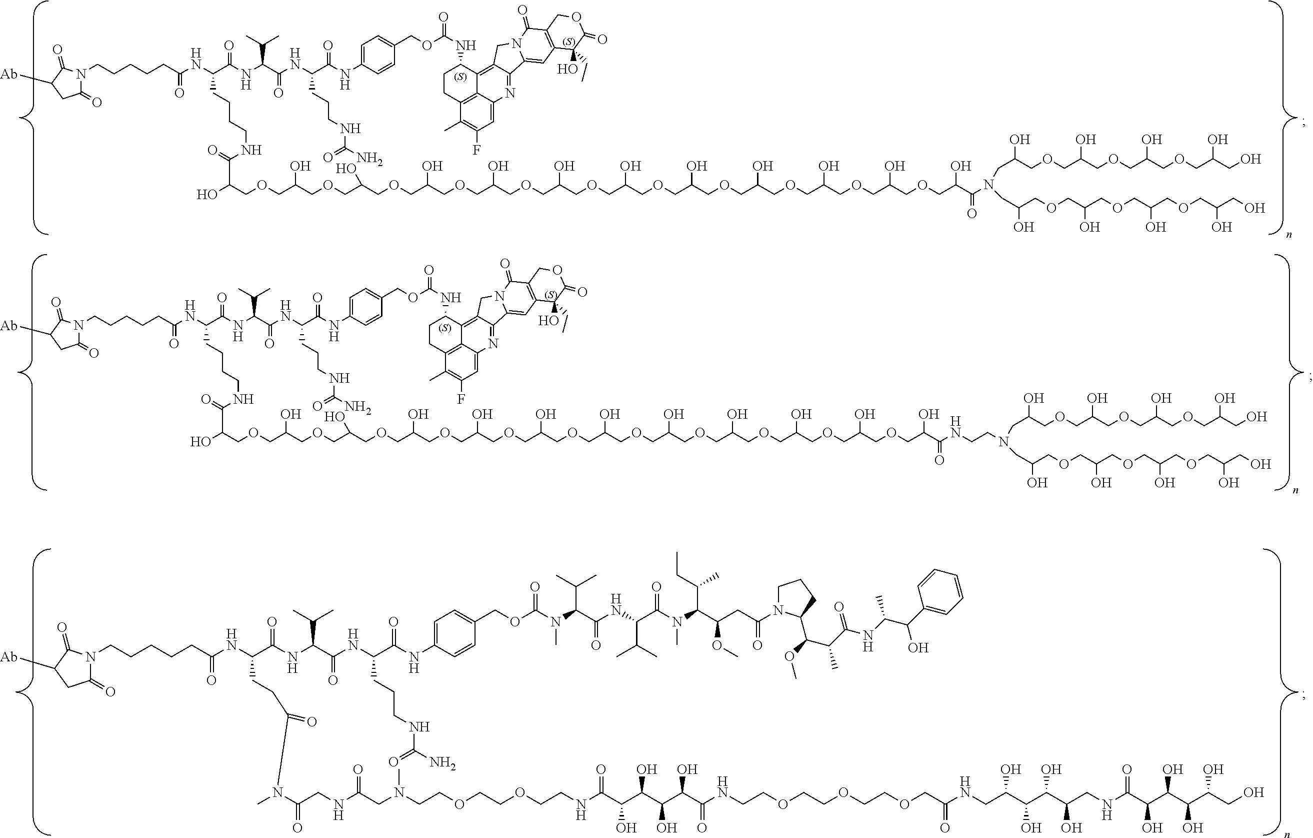 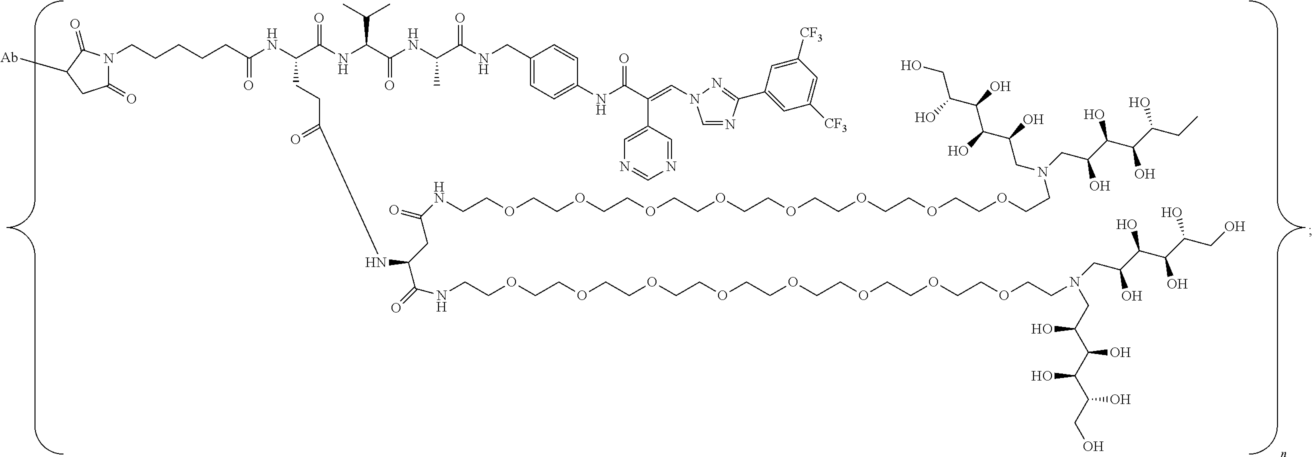 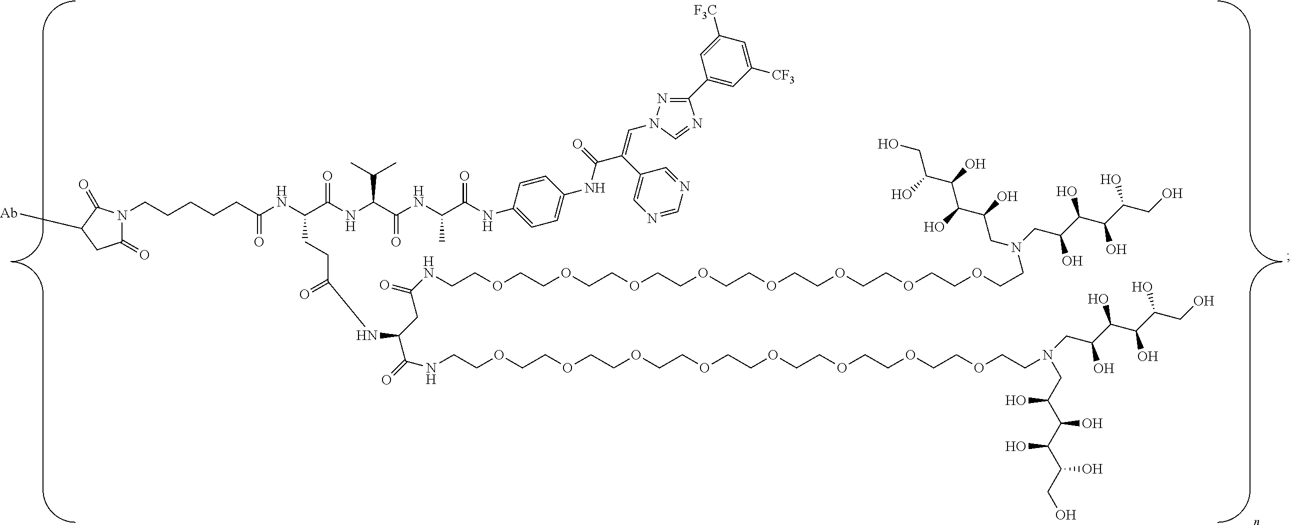 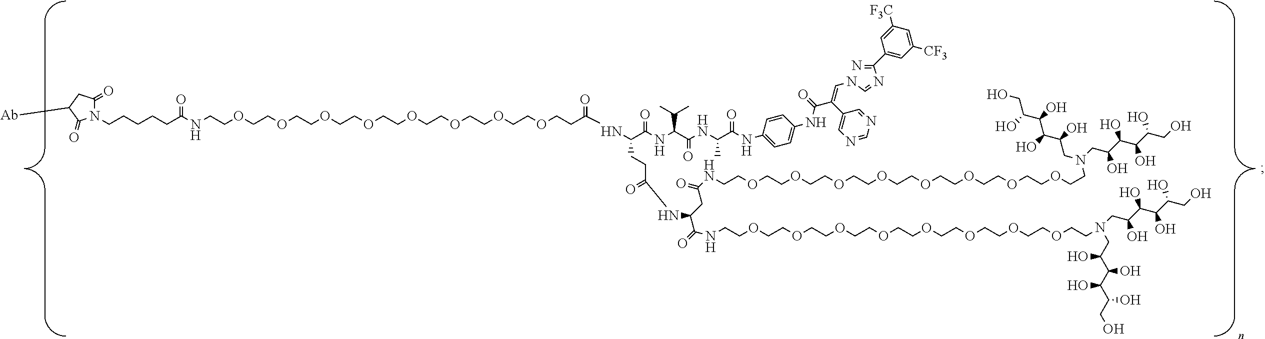 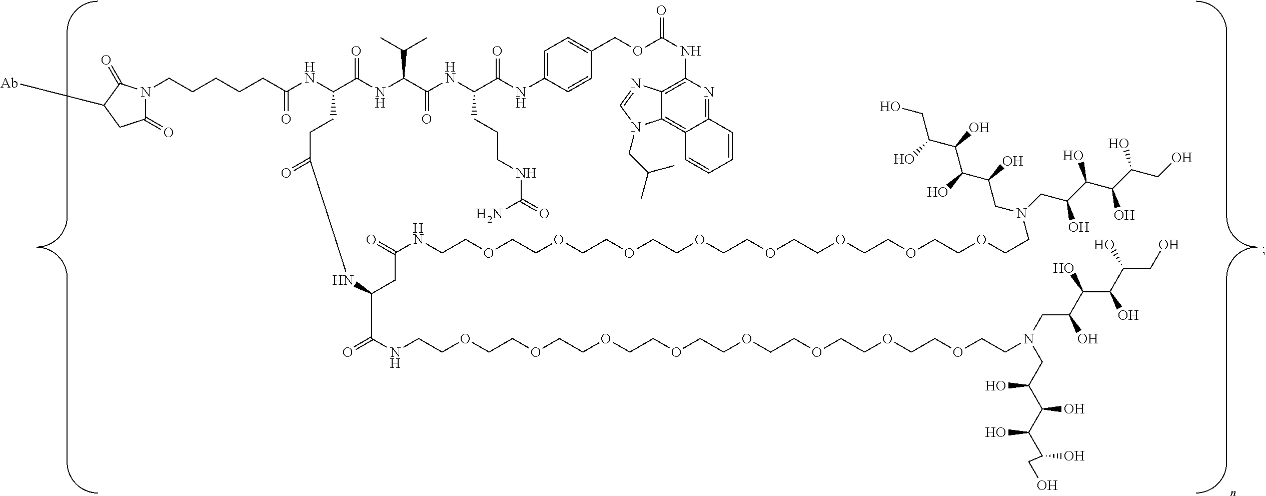 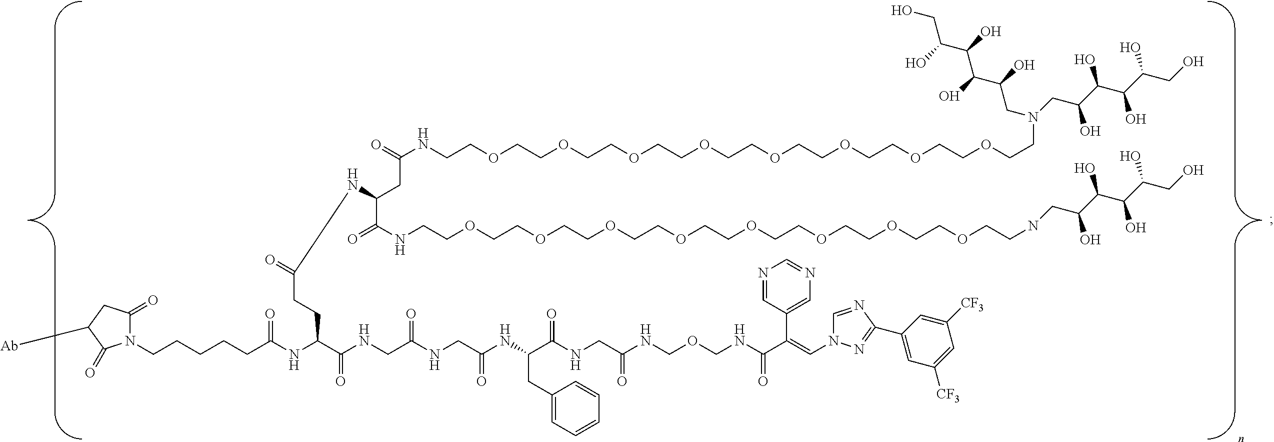 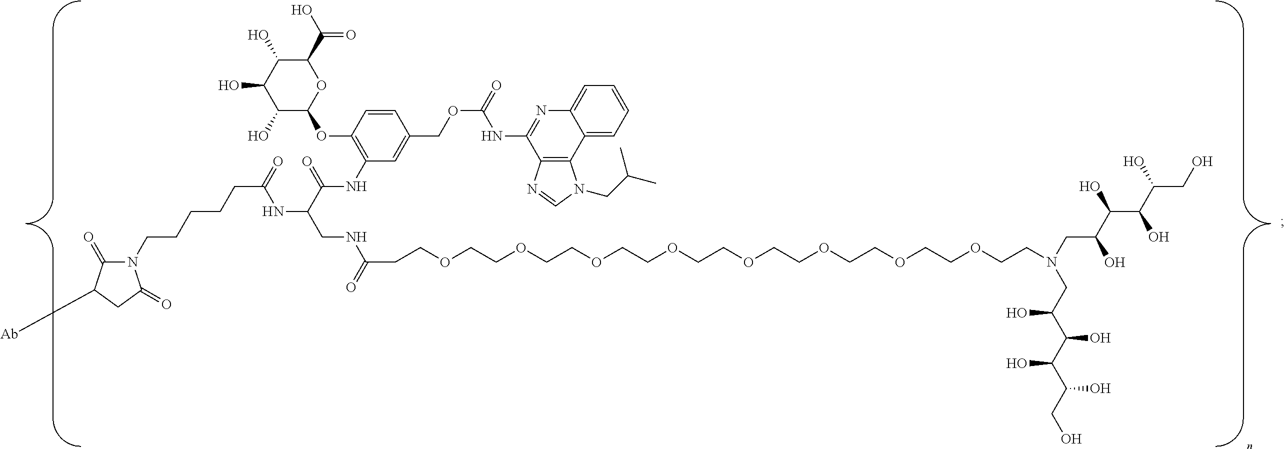 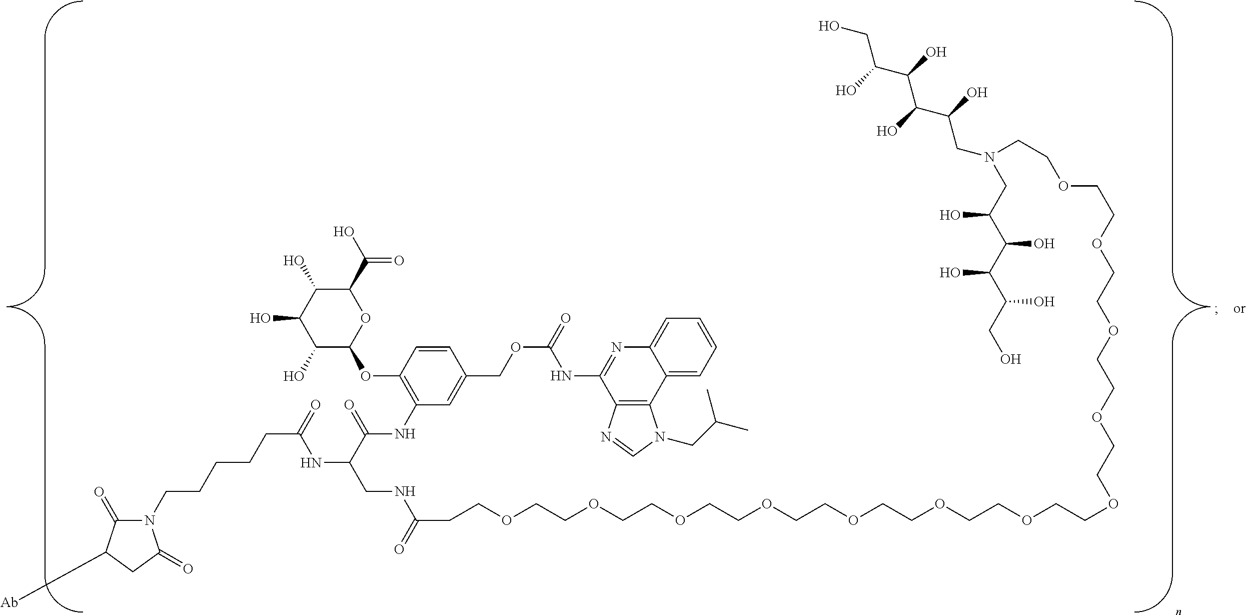 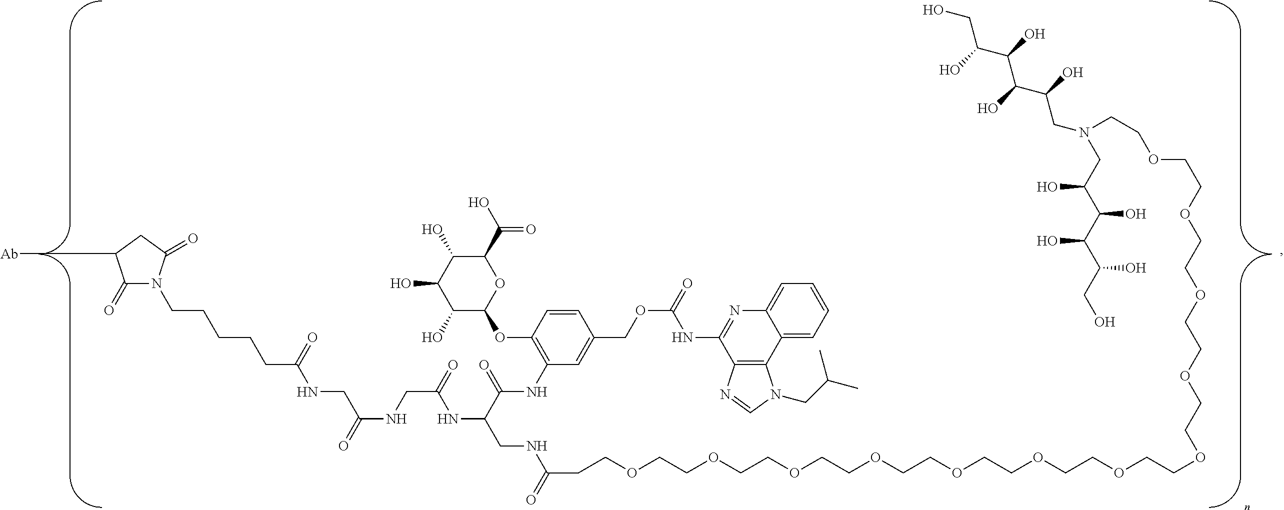
or a stereoisomer thereof, wherein Ab is the bispecific binding agent and n is p^{load}.

58. The conjugate of claim 23, wherein the conjugate has the following structure: 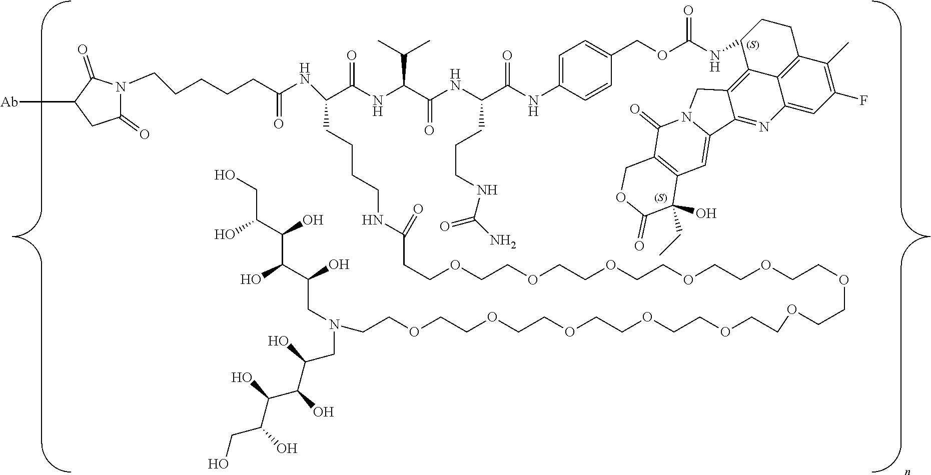
or a stereoisomer thereof, wherein Ab is the bispecific binding agent and n is p^{load} wherein p^{load} is from about 1 to about 8, about 2, about 4, about 6, about 8, about 10, about 12, about 14, about 16, about 3 to about 5, about 6 to about 8, or about 8 to about 16.

59. A pharmaceutical composition comprising the conjugate of claim 23 and a pharmaceutically acceptable carrier.

60. A method of treating an EGFR+ and/or c-MET+ cancer, comprising administering to a subject in need thereof a therapeutically effective amount of the bispecific binding agent of claim 1.

61-74. (canceled)

75. A method of treating an autoimmune disease, comprising administering to a subject in need thereof a therapeutically effective amount of the conjugate of claim 23.

76-82. (canceled)


SEQUENCE LISTING (271 sequences)

SEQ ID NO: 1 - protein, 119 aa, synthetic construct [sequence omitted]

SEQ ID NO: 2 - protein, 113 aa, synthetic construct [sequence omitted]

SEQ ID NO: 3 - protein, 8 aa, synthetic construct
GYTFTSYW

SEQ ID NO: 4 - protein, 8 aa, synthetic construct
IDPSNSDT

SEQ ID NO: 5 - protein, 12 aa, synthetic construct
ATYRSYVTPLDY

SEQ ID NO: 6 - protein, 12 aa, synthetic construct
QSLLYTSSQKNY

SEQ ID NO: 7 - protein, 9 aa, synthetic construct
QQYYAYPWT

SEQ ID NO: 8 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 9 - protein, 111 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 10 - protein, 8 aa, synthetic construct
GYIFTAYT

SEQ ID NO: 11 - protein, 8 aa, synthetic construct
IKPNNGLA

SEQ ID NO: 12 - protein, 11 aa, synthetic construct
ARSEITTEFDY

SEQ ID NO: 13 - protein, 10 aa, synthetic construct
ESVDSYANSF

SEQ ID NO: 14 - protein, 9 aa, synthetic construct
QQSKEDPLT

SEQ ID NO: 15 - protein, 115 aa, synthetic construct [sequence omitted]

SEQ ID NO: 16 - protein, 108 aa, synthetic construct [sequence omitted]

SEQ ID NO: 17 - protein, 8 aa, synthetic construct
GYTFTDYY

SEQ ID NO: 18 - protein, 8 aa, synthetic construct
VNPNRRGT

SEQ ID NO: 19 - protein, 8 aa, synthetic construct
ARANWLDY

SEQ ID NO: 20 - protein, 7 aa, synthetic construct
SSVSSIY

SEQ ID NO: 21 - protein, 9 aa, synthetic construct
QVYSGYPLT

SEQ ID NO: 22 - protein, 121 aa, synthetic construct
QVQLVQSGAEVKKPGASVKVSCKASGYTFTGYYMNWVRQAPGQGLEWMGIINPWTGNTNYAQKFQGRVTMTRDTSISTAYMELSSLRSEDTAVYYCARDPGFFYYTPSDLWGQGTLVTVSS

SEQ ID NO: 23 - protein, 109 aa, synthetic construct
DIELTQPPSVSVAPGQTARISCSGDSIGNKYVHWYQQKPGQAPVLVIYADSDRPSGIPERFSGSNSGNTATLTISGTQAEDEADYYCQSYAHYHDIWVFGGGTKLTVLG

SEQ ID NO: 24 - protein, 8 aa, synthetic construct
GYTFTGYY

SEQ ID NO: 25 - protein, 8 aa, synthetic construct
INPWTGNT

SEQ ID NO: 26 - protein, 14 aa, synthetic construct
ARDPGFFYYTPSDL

SEQ ID NO: 27 - protein, 6 aa, synthetic construct
SIGNKY

SEQ ID NO: 28 - protein, 11 aa, synthetic construct
QSYAHYHDIWV

SEQ ID NO: 29 - protein, 113 aa, synthetic construct
QVQLVQSGAEVKKPGSSVKVSCKASGGTFSSYAISWVRQAPGQGLEWMGGIDPFGTANYAQKFQGRVTITADESTSTAYMELSSLRSEDTAVYYCARVYQDVWGQGTLVTVSS

SEQ ID NO: 30 - protein, 111 aa, synthetic construct
DIVMTQSPDSLAVSLGERATINCRSSQSILYGINNNFLGWYQQKPGQPPKLLIYWASTRESGVPDRFSGSGSGTDFTLTISSLQAEDVAVYYCQQYAFGWTFGQGTKVEIK

SEQ ID NO: 31 - protein, 8 aa, synthetic construct
GGTFSSYA

SEQ ID NO: 32 - protein, 7 aa, synthetic construct
IDPFGTA

SEQ ID NO: 33 - protein, 7 aa, synthetic construct
ARVYQDV

SEQ ID NO: 34 - protein, 11 aa, synthetic construct
QSILYGINNNF

SEQ ID NO: 35 - protein, 8 aa, synthetic construct
QQYAFGWT

SEQ ID NO: 36 - protein, 113 aa, synthetic construct; identical to SEQ ID NO: 29

SEQ ID NO: 37 - protein, 112 aa, synthetic construct
DIVMTQSPDSLAVSLGERATINCRSSQSILYGINNNFLGWYQQKPGQPPKLLIYWASTRESGVPDRFSGSGSGTDFTLTISSLQAEDVAVYYCLQYSDEPWTFGQGTKVEIK

SEQ ID NO: 38 - protein, 8 aa, synthetic construct
GGTFSSYA

SEQ ID NO: 39 - protein, 7 aa, synthetic construct
IDPFGTA

SEQ ID NO: 40 - protein, 7 aa, synthetic construct
ARVYQDV

SEQ ID NO: 41 - protein, 11 aa, synthetic construct
QSILYGINNNF

SEQ ID NO: 42 - protein, 9 aa, synthetic construct
LQYSDEPWT

SEQ ID NO: 43 - protein, 113 aa, synthetic construct
QVQLVQSGAEVKKPGSSVKVSCKASGGTFSSYAISWVRQAPGQGLEWMGGIDPIMGTEYAQKFQGRVTITADESTSTAYMELSSLRSEDTAVYYCARVYQDVWGQGTLVTVSS

SEQ ID NO: 44 - protein, 112 aa, synthetic construct
DIVMTQSPDSLAVSLGERATINCRSSQSILYGINNNFLGWYQQKPGQPPKLLIYWASTRESGVPDRFSGSGSGTDFTLTISSLQAEDVAVYYCQQYAYEPNTFGQGTKVEIK

SEQ ID NO: 45 - protein, 8 aa, synthetic construct
GGTFSSYA

SEQ ID NO: 46 - protein, 7 aa, synthetic construct
IDPIMGT

SEQ ID NO: 47 - protein, 7 aa, synthetic construct
ARVYQDV

SEQ ID NO: 48 - protein, 11 aa, synthetic construct
QSILYGINNNF

SEQ ID NO: 49 - protein, 9 aa, synthetic construct
QQYAYEPNT

SEQ ID NO: 50 - protein, 117 aa, synthetic construct
EVKLVESGGGLVQPGGSLRLSCATSGFTFTDYYMSWVRQPPGKALEWLGFIRNKANGYTTEYSASVKGRFTISRDNSQSILYLQMDTLRAEDSATYYCARDNWFAYWGQGTLVTVSA

SEQ ID NO: 51 - protein, 113 aa, synthetic construct
DILMTQSPSSLTVSAGEKVTMSCKSSQSLLASGNQNNYLAWHQQKPGRSPKMLIIWASTRVSGVPDRFIGSGSGTDFTLTINSVQAEDLAVYYCQQSYSAPLTFGAGTKLELK

SEQ ID NO: 52 - protein, 8 aa, synthetic construct
GFTFTDYY

SEQ ID NO: 53 - protein, 10 aa, synthetic construct
IRNKANGYTT

SEQ ID NO: 54 - protein, 8 aa, synthetic construct
ARDNWFAY

SEQ ID NO: 55 - protein, 12 aa, synthetic construct
QSLLASGNQNNY

SEQ ID NO: 56 - protein, 9 aa, synthetic construct
QQSYSAPLT

SEQ ID NO: 57 - protein, 119 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 58 - protein, 107 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 59 - protein, 8 aa, synthetic construct
GYTFTSYG

SEQ ID NO: 60 - protein, 8 aa, synthetic construct
ISAYNGYT

SEQ ID NO: 61 - protein, 12 aa, synthetic construct
ARDLRGTNYFDY

SEQ ID NO: 62 - protein, 6 aa, synthetic construct
QGISNW

SEQ ID NO: 63 - protein, 9 aa, synthetic construct
QQANSFPIT

SEQ ID NO: 64 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 65 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 66 - protein, 8 aa, synthetic construct
GFSLTNYG

SEQ ID NO: 67 - protein, 7 aa, synthetic construct
IWSGGNT

SEQ ID NO: 68 - protein, 13 aa, synthetic construct
ARALTYYDYEFAY

SEQ ID NO: 69 - protein, 6 aa, synthetic construct
QSIGTN

SEQ ID NO: 70 - protein, 9 aa, synthetic construct
QQNNNWPTT

SEQ ID NO: 71 - protein, 119 aa, synthetic construct [sequence omitted]

SEQ ID NO: 72 - protein, 107 aa, synthetic construct [sequence omitted]

SEQ ID NO: 73 - protein, 10 aa, synthetic construct
GGSVSSGDYY

SEQ ID NO: 74 - protein, 7 aa, synthetic construct
IYYSGNT

SEQ ID NO: 75 - protein, 11 aa, synthetic construct
VRDRVTGAFDI

SEQ ID NO: 76 - protein, 6 aa, synthetic construct
QDISNY

SEQ ID NO: 77 - protein, 9 aa, synthetic construct
QHFDHLPLA

SEQ ID NO: 78 - protein, 123 aa, synthetic construct [sequence omitted]

SEQ ID NO: 79 - protein, 112 aa, synthetic construct [sequence omitted]

SEQ ID NO: 80 - protein, 8 aa, synthetic construct
GYTFTNYY

SEQ ID NO: 81 - protein, 8 aa, synthetic construct
INPTSGGS

SEQ ID NO: 82 - protein, 16 aa, synthetic construct
TRQGLWFDSDGRGFDF

SEQ ID NO: 83 - protein, 11 aa, synthetic construct
QNIVHSNGNTY

SEQ ID NO: 84 - protein, 9 aa, synthetic construct
FQYSHVPWT

SEQ ID NO: 85 - protein, 125 aa, synthetic construct [sequence omitted]

SEQ ID NO: 86 - protein, 107 aa, synthetic construct [sequence omitted]

SEQ ID NO: 87 - protein, 8 aa, synthetic construct
GFTFSTYG

SEQ ID NO: 88 - protein, 8 aa, synthetic construct
IWDDGSYK

SEQ ID NO: 89 - protein, 18 aa, synthetic construct
ARDGITMVRGVMKDYFDY

SEQ ID NO: 90 - protein, 6 aa, synthetic construct
QDISSA

SEQ ID NO: 91 - protein, 9 aa, synthetic construct
QQFNSYPLT

SEQ ID NO: 92 - protein, 121 aa, synthetic construct [sequence omitted]

SEQ ID NO: 93 - protein, 107 aa, synthetic construct [sequence omitted]

SEQ ID NO: 94 - protein, 10 aa, synthetic construct
GGSISSGDYY

SEQ ID NO: 95 - protein, 7 aa, synthetic construct
IYYSGST

SEQ ID NO: 96 - protein, 13 aa, synthetic construct
ARVSIFGVGTFDY

SEQ ID NO: 97 - protein, 6 aa, synthetic construct
QSVSSY

SEQ ID NO: 98 - protein, 9 aa, synthetic construct
HQYGSTPLT

SEQ ID NO: 99 - protein, 119 aa, synthetic construct
QVQLQESGPGLVKPSETLSLTCTVSGFSLSNYDVHWVRQAPGKGLEWLGVIWSGGNTDYNTPFTSRLTISVDTSKNQFSLKLSSVTAADTAVYYCARALDYYDYEFAYWGQGTLVTVSS

SEQ ID NO: 100 - protein, 107 aa, synthetic construct
EIVLTQSPDFQSVTPKEKVTITCRASQSIGTNIHWYQQKPDQSPKLLIKYASESISGIPSRFSGSGSGTDFTLTINSLEAEDAATYYCQQNNEWPTSFGQGTKLEIK

SEQ ID NO: 101 - protein, 8 aa, synthetic construct
GFSLSNYD

SEQ ID NO: 102 - protein, 7 aa, synthetic construct
IWSGGNT

SEQ ID NO: 103 - protein, 13 aa, synthetic construct
ARALDYYDYEFAY

SEQ ID NO: 104 - protein, 6 aa, synthetic construct
QSIGTN

SEQ ID NO: 105 - protein, 9 aa, synthetic construct
QQNNEWPTS

SEQ ID NO: 106 - protein, 455 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 107 - protein, 214 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 8 aa, synthetic construct
GFTFSTYG

SEQ ID NO: 109 - protein, 8 aa, synthetic construct
IWDDGSYK

SEQ ID NO: 110 - protein, 18 aa, synthetic construct
ARDGITMVRGVMKDYFDY

SEQ ID NO: 111 - protein, 6 aa, synthetic construct
QDISSA

SEQ ID NO: 112 - protein, 9 aa, synthetic construct
QQFNSYPLT

SEQ ID NO: 113 - protein, 449 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 114 - protein, 214 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 8 aa, synthetic construct
GYTFTSYG

SEQ ID NO: 116 - protein, 8 aa, synthetic construct
ISAYNGYT

SEQ ID NO: 117 - protein, 12 aa, synthetic construct
ARDLRGTNYFDY

SEQ ID NO: 118 - protein, 6 aa, synthetic construct
QGISNW

SEQ ID NO: 119 - protein, 9 aa, synthetic construct
QQANSFPIT

SEQ ID NO: 120 - protein, 454 aa, synthetic construct
QVQLVQSGAEVKKPGSSVKVSCKASGGTFSDNDFSWVRQAPGQGLEWMGAIVAVFRTETYAQKFQDRVKITADISTRTTYMELSSLRSEDTAVYYCARRLMSAISGPGAPLLMWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 217 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSDTLEIFGGGTKLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 122 - protein, 8 aa, synthetic construct
GGTFSDND

SEQ ID NO: 123 - protein, 8 aa, synthetic construct
IVAVFRTE

SEQ ID NO: 124 - protein, 17 aa, synthetic construct
ARRLMSAISGPGAPLLM

SEQ ID NO: 125 - protein, 9 aa, synthetic construct
SSDVGGYNY

SEQ ID NO: 126 - protein, 11 aa, synthetic construct
SSYTSSDTLEI

SEQ ID NO: 127 - protein, 446 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 128 - protein, 214 aa, synthetic construct
DIQMTQSPSTLSASVGDRVTITCRASEGIYHWLAWYQQKPGKAPKLLIYKASSLASGVPSRFSGSGSGTEFTLTISSLQPDDFATYYCQQYSNYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 8 aa, synthetic construct
GYTFTDYY

SEQ ID NO: 130 - protein, 8 aa, synthetic construct
MNPNSGNT

SEQ ID NO: 131 - protein, 9 aa, synthetic construct
ARGQGYTHS

SEQ ID NO: 132 - protein, 6 aa, synthetic construct
EGIYHW

SEQ ID NO: 133 - protein, 9 aa, synthetic construct
QQYSNYPPT

SEQ ID NO: 134 - protein, 709 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 455 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 214 aa, synthetic construct; identical to SEQ ID NO: 107

SEQ ID NO: 137 - protein, 125 aa, synthetic construct [sequence omitted]

SEQ ID NO: 138 - protein, 107 aa, synthetic construct [sequence omitted]

SEQ ID NO: 139 - protein, 8 aa, synthetic construct
GFTFSTYG

SEQ ID NO: 140 - protein, 8 aa, synthetic construct
IWDDGSYK

SEQ ID NO: 141 - protein, 18 aa, synthetic construct
ARDGITMVRGVMKDYFDY

SEQ ID NO: 142 - protein, 6 aa, synthetic construct
QDISSA

SEQ ID NO: 143 - protein, 9 aa, synthetic construct
QQFNSYPLT

SEQ ID NO: 144 - protein, 706 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 145 - protein, 448 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 146 - protein, 218 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 118 aa, synthetic construct; identical to SEQ ID NO: 8

SEQ ID NO: 148 - protein, 111 aa, synthetic construct; identical to SEQ ID NO: 9

SEQ ID NO: 149 - protein, 8 aa, synthetic construct
GYIFTAYT

SEQ ID NO: 150 - protein, 8 aa, synthetic construct
IKPNNGLA

SEQ ID NO: 151 - protein, 11 aa, synthetic construct
ARSEITTEFDY

SEQ ID NO: 152 - protein, 10 aa, synthetic construct
ESVDSYANSF

SEQ ID NO: 153 - protein, 9 aa, synthetic construct
QQSKEDPLT

SEQ ID NO: 154 - protein, 709 aa, synthetic construct; identical to SEQ ID NO: 134

SEQ ID NO: 155 - protein, 455 aa, synthetic construct; identical to SEQ ID NO: 135

SEQ ID NO: 156 - protein, 214 aa, synthetic construct; identical to SEQ ID NO: 107

SEQ ID NO: 157 - protein, 125 aa, synthetic construct [sequence omitted]

SEQ ID NO: 158 - protein, 107 aa, synthetic construct [sequence omitted]

SEQ ID NO: 159 - protein, 709 aa, synthetic construct
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 160 - protein, 449 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 161 - protein, 220 aa, synthetic construct
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 119 aa, synthetic construct [sequence omitted]

SEQ ID NO: 163 - protein, 113 aa, synthetic construct [sequence omitted]

SEQ ID NO: 164 - protein, 10 aa, synthetic construct
GYTFTSYWLH

SEQ ID NO: 165 - protein, 8 aa, synthetic construct
IDPSNSDT

SEQ ID NO: 166 - protein, 12 aa, synthetic construct
ATYRSYVTPLDY

SEQ ID NO: 167 - protein, 12 aa, synthetic construct
QSLLYTSSQKNY

SEQ ID NO: 168 - protein, 9 aa, synthetic construct
QQYYAYPWT

SEQ ID NO: 169 - protein, 712 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 453 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 219 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 123 aa, synthetic construct [sequence omitted]

SEQ ID NO: 173 - protein, 112 aa, synthetic construct [sequence omitted]

SEQ ID NO: 174 - protein, 8 aa, synthetic construct
GYTFTNYY

SEQ ID NO: 175 - protein, 8 aa, synthetic construct
INPTSGGS

SEQ ID NO: 176 - protein, 16 aa, synthetic construct
TRQGLWFDSDGRGFDF

SEQ ID NO: 177 - protein, 11 aa, synthetic construct
QNIVHSNGNTY

SEQ ID NO: 178 - protein, 9 aa, synthetic construct
FQYSHVPWT

SEQ ID NO: 179 - protein, 706 aa, synthetic construct; identical to SEQ ID NO: 144

SEQ ID NO: 180 - protein, 448 aa, synthetic construct; identical to SEQ ID NO: 145

SEQ ID NO: 181 - protein, 218 aa, synthetic construct; identical to SEQ ID NO: 146

SEQ ID NO: 182 - protein, 118 aa, synthetic construct; identical to SEQ ID NO: 8

SEQ ID NO: 183 - protein, 111 aa, synthetic construct; identical to SEQ ID NO: 9

SEQ ID NO: 184 - protein, 712 aa, synthetic construct; identical to SEQ ID NO: 169

SEQ ID NO: 185 - protein, 453 aa, synthetic construct; identical to SEQ ID NO: 170

SEQ ID NO: 186 - protein, 219 aa, synthetic construct; identical to SEQ ID NO: 171

SEQ ID NO: 187 - protein, 123 aa, synthetic construct [sequence omitted]

SEQ ID NO: 188 - protein, 112 aa, synthetic construct [sequence omitted]

SEQ ID NO: 189 - protein, 709 aa, synthetic construct
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSEVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 190 - protein, 449 aa, synthetic construct
EVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 191 - protein, 220 aa, synthetic construct; identical to SEQ ID NO: 161

SEQ ID NO: 192 - protein, 119 aa, synthetic construct
EVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSS

SEQ ID NO: 193 - protein, 113 aa, synthetic construct [sequence omitted]

SEQ ID NO: 194 - protein, 8 aa, synthetic construct
GYTFTSYW

SEQ ID NO: 195 - protein, 709 aa, synthetic construct
AIQLTQSPESLSASVGDRVTITCRASQDISSALVWYQQKPGKAPKLLIYDASSLESGVPDRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFSTYGMHWVRQAPGQGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 455 aa, synthetic construct
QVQLVESGGGVVQPGRSLRLSCAASGFTFSTYGMHWVRQAPGQGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 214 aa, synthetic construct
AIQLTQSPESLSASVGDRVTITCRASQDISSALVWYQQKPGKAPKLLIYDASSLESGVPDRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 125 aa, synthetic construct
QVQLVESGGGVVQPGRSLRLSCAASGFTFSTYGMHWVRQAPGQGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 199 - protein, 107 aa, synthetic construct
AIQLTQSPESLSASVGDRVTITCRASQDISSALVWYQQKPGKAPKLLIYDASSLESGVPDRFSGSESGTDFTLTISSLQPEDFATYYCQQFNSYPLTFGGGTKVEIK

SEQ ID NO: 200 - protein, 706 aa, synthetic construct; identical to SEQ ID NO: 144

SEQ ID NO: 201 - protein, 448 aa, synthetic construct; identical to SEQ ID NO: 145

SEQ ID NO: 202 - protein, 218 aa, synthetic construct; identical to SEQ ID NO: 146

SEQ ID NO: 203 - protein, 118 aa, synthetic construct; identical to SEQ ID NO: 8

SEQ ID NO: 204 - protein, 111 aa, synthetic construct; identical to SEQ ID NO: 9

SEQ ID NO: 205 - protein, 709 aa, synthetic construct
AIQLTQSPESLSASVGDRVTITCRASQDISSALVWYQQKPGKAPKLLIYDASSLESGVPDRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFSTYGMHWVRQAPGEGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 455 aa, synthetic construct
QVQLVESGGGVVQPGRSLRLSCAASGFTFSTYGMHWVRQAPGEGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 214 aa, synthetic construct; identical to SEQ ID NO: 197

SEQ ID NO: 208 - protein, 125 aa, synthetic construct
QVQLVESGGGVVQPGRSLRLSCAASGFTFSTYGMHWVRQAPGEGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 209 - protein, 107 aa, synthetic construct; identical to SEQ ID NO: 199

SEQ ID NO: 210 - protein, 706 aa, synthetic construct; identical to SEQ ID NO: 144

SEQ ID NO: 211 - protein, 448 aa, synthetic construct; identical to SEQ ID NO: 145

SEQ ID NO: 212 - protein, 218 aa, synthetic construct; identical to SEQ ID NO: 146

SEQ ID NO: 213 - protein, 118 aa, synthetic construct; identical to SEQ ID NO: 8

SEQ ID NO: 214 - protein, 111 aa, synthetic construct; identical to SEQ ID NO: 9

SEQ ID NO: 215 - protein, 709 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 455 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 214 aa, synthetic construct; identical to SEQ ID NO: 107

SEQ ID NO: 218 - protein, 125 aa, synthetic construct [sequence omitted]

SEQ ID NO: 219 - protein, 107 aa, synthetic construct [sequence omitted]

SEQ ID NO: 220 - protein, 706 aa, synthetic construct; identical to SEQ ID NO: 144

SEQ ID NO: 221 - protein, 448 aa, synthetic construct; identical to SEQ ID NO: 145

SEQ ID NO: 222 - protein, 218 aa, synthetic construct; identical to SEQ ID NO: 146

SEQ ID NO: 223 - protein, 118 aa, synthetic construct; identical to SEQ ID NO: 8

SEQ ID NO: 224 - protein, 111 aa, synthetic construct; identical to SEQ ID NO: 9

SEQ ID NO: 225 - protein, 709 aa, synthetic construct; identical to SEQ ID NO: 215

SEQ ID NO: 226 - protein, 455 aa, synthetic construct; identical to SEQ ID NO: 216

SEQ ID NO: 227 - protein, 214 aa, synthetic construct; identical to SEQ ID NO: 107

SEQ ID NO: 228 - protein, 125 aa, synthetic construct [sequence omitted]

SEQ ID NO: 229 - protein, 107 aa, synthetic construct [sequence omitted]

SEQ ID NO: 230 - protein, 703 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 231 - protein, 449 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 232 - protein, 214 aa, synthetic construct; identical to SEQ ID NO: 114

SEQ ID NO: 233 - protein, 119 aa, synthetic construct; identical to SEQ ID NO: 57

SEQ ID NO: 234 - protein, 107 aa, synthetic construct; identical to SEQ ID NO: 58

SEQ ID NO: 235 - protein, 8 aa, synthetic construct
GYTFTSYG

SEQ ID NO: 236 - protein, 8 aa, synthetic construct
ISAYNGYT

SEQ ID NO: 237 - protein, 12 aa, synthetic construct
ARDLRGTNYFDY

SEQ ID NO: 238 - protein, 7 aa, synthetic construct
QGISNWL

SEQ ID NO: 239 - protein, 9 aa, synthetic construct
QQANSFPIT

SEQ ID NO: 240 - protein, 709 aa, synthetic construct
AIQLTQSPESLSASVGDRVTITCRASQDISSALVWYQQKPGKAPKLLIYDASSLESGVPDRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFSTYGMHWVRQAPGQGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 455 aa, synthetic construct
QVQLVESGGGVVQPGRSLRLSCAASGFTFSTYGMHWVRQAPGQGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 214 aa, synthetic construct; identical to SEQ ID NO: 197

SEQ ID NO: 243 - protein, 125 aa, synthetic construct; identical to SEQ ID NO: 198

SEQ ID NO: 244 - protein, 107 aa, synthetic construct; identical to SEQ ID NO: 199

SEQ ID NO: 245 - protein, 706 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 246 - protein, 448 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 247 - protein, 218 aa, synthetic construct; identical to SEQ ID NO: 146

SEQ ID NO: 248 - protein, 118 aa, synthetic construct; identical to SEQ ID NO: 8

SEQ ID NO: 249 - protein, 111 aa, synthetic construct; identical to SEQ ID NO: 9

SEQ ID NO: 250 - protein, 489 aa, synthetic construct
QVQLVESGGGVVQPGRSLRLSCAASGFTFSTYGMHWVREAPGKDLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSGGGGSAIQLTQSPSSLSASVGDRVTITCRASQDISSALVWYQKKPGKAPKLLIYDASSLESGVPSRFSGSESGTDFTLTISSLQPEDFATYYCQQFNSYPLTFGKGTKVEIKGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 125 aa, synthetic construct
QVQLVESGGGVVQPGRSLRLSCAASGFTFSTYGMHWVREAPGKDLEWVAVIWDDGSYKYYGDSVKGRFTISRDNSKNTLYLQMNSLRAEDTAVYYCARDGITMVRGVMKDYFDYWGQGTLVTVSS

SEQ ID NO: 252 - protein, 107 aa, synthetic construct
AIQLTQSPSSLSASVGDRVTITCRASQDISSALVWYQKKPGKAPKLLIYDASSLESGVPSRFSGSESGTDFTLTISSLQPEDFATYYCQQFNSYPLTFGKGTKVEIK

SEQ ID NO: 253 - protein, 486 aa, synthetic construct
QVQLVQSGAEVKKPGASVKVSCKASGYIFTAYTMHWVRQAPGQKLEWMGWIKPNNGLANYAQKFQGRVTMTRDTSISTAYMELSRLRSDDTAVYYCARSEITTEFDYWGQGTLVTVSSGGGGSGGGGSGGGGSGGGGSDIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGDGTKVEIKGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 254 - protein, 118 aa, synthetic construct
QVQLVQSGAEVKKPGASVKVSCKASGYIFTAYTMHWVRQAPGQKLEWMGWIKPNNGLANYAQKFQGRVTMTRDTSISTAYMELSRLRSDDTAVYYCARSEITTEFDYWGQGTLVTVSS

SEQ ID NO: 255 - protein, 111 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGDGTKVEIK

SEQ ID NO: 256 - protein, 709 aa, synthetic construct
AIQLTQSPESLSASVGDRVTITCRASQDISSALVWYQQKPGKAPKLLIYDASSLESGVPDRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFSTYGMHWVRQAPGEGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 455 aa, synthetic construct
QVQLVESGGGVVQPGRSLRLSCAASGFTFSTYGMHWVRQAPGEGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 214 aa, synthetic construct; identical to SEQ ID NO: 197

SEQ ID NO: 259 - protein, 125 aa, synthetic construct; identical to SEQ ID NO: 208

SEQ ID NO: 260 - protein, 107 aa, synthetic construct; identical to SEQ ID NO: 199

SEQ ID NO: 261 - protein, 706 aa, synthetic construct; identical to SEQ ID NO: 144

SEQ ID NO: 262 - protein, 448 aa, synthetic construct; identical to SEQ ID NO: 145

SEQ ID NO: 263 - protein, 218 aa, synthetic construct; identical to SEQ ID NO: 146

SEQ ID NO: 264 - protein, 118 aa, synthetic construct; identical to SEQ ID NO: 8

SEQ ID NO: 265 - protein, 111 aa, synthetic construct; identical to SEQ ID NO: 9

SEQ ID NO: 266 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 330 aa, synthetic construct; identical to SEQ ID NO: 266

SEQ ID NO: 268 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 269 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 270 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 271 - protein, 107 aa, synthetic construct [sequence omitted]